(19) United States
(12) Patent Application Publication
(10) Pub. No.: US 2010/0105655 A1
(43) Pub. Date: Apr. 29, 2010

(54) NOVEL COMPOUNDS 515

(75) Inventors: Richard Ducray, Reims (FR); Clifford David Jones, Reims (FR); Frédéric Henri Jung, Reims (GB); Iain Simpson, Macclesfield (GB)

Correspondence Address: ASTRAZENECA R&D BOSTON, 35 GATEHOUSE DRIVE, WALTHAM, MA, 02451-1215, US

(73) Assignee: ASTRAZENECA AB

(21) Appl. No.: 12/607,505

(22) Filed: Oct. 28, 2009

(30) Foreign Application Priority Data
Oct. 29, 2008 (EP) 08305748.9

(51) Int. Cl. A61K 31/397; C07D 403/14; A61K 31/496; C07D 413/14; A61K 31/5377; C07D 401/14; A61K 31/506; A61K 31/498; C07D 498/02; A61K 31/5365; A61P 35/00

(52) U.S. Cl. 514/210.21; 544/295; 514/252.18; 544/122; 514/233.2; 544/331; 514/275; 514/249; 544/105; 514/230.5

(57) ABSTRACT

There is provided novel pyrimidine derivatives of formula (I)

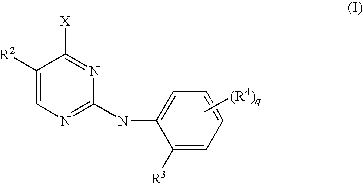

or pharmaceutically acceptable salts thereof, processes for their preparation, pharmaceutical compositions containing them and their use in therapy.

This application claims the benefit under 35 U.S.C. §119(a)-(d) of Application No EP08305748.9 filed on 29 Oct. 2008.

The present invention relates to novel pyrimidine derivatives, processes for their preparation, pharmaceutical compositions containing them and their use in therapy.

The insulin-like growth factor (IGF) axis consists of ligands, receptors, binding proteins and proteases. The two ligands, IGF-I and IGF-II, are mitogenic peptides that signal through interaction with the type 1 insulin-like growth factor receptor (IGF-1R), a hetero-tetrameric cell surface receptor. Binding of either ligand stimulates activation of a tyrosine kinase domain in the intracellular region of the β-chain and results in phosphorylation of several tyrosine residues resulting in the recruitment and activation of various signalling molecules. The intracellular domain has been shown to transmit signals for mitogenesis, survival, transformation, and differentiation in cells. The structure and function of the IGF-1R has been reviewed by Adams et al (Cellular and Molecular Life Sciences, 57, 1050-1093, 2000). The IGF-IIR (also known as mannose 6-phosphate receptor) has no such kinase domain and does not signal mitogenesis but may act to regulate ligand availability at the cell surface, counteracting the effect of the IGF-1R. The IGF binding proteins (IGFBP) control availability of circulating IGF and release of IGF from these can be mediated by proteolytic cleavage. These other components of the IGF axis have been reviewed by Collett-Solberg and Cohen (Endocrine, 12, 121-136, 2000).

There is considerable evidence linking IGF signalling with cellular transformation and the onset and progression of tumours. IGF has been identified as the major survival factor that protects from oncogene induced cell death (Harrington et al, EMBO J, 13, 3286-3295, 1994). Cells lacking IGF-1R have been shown to be refractory to transformation by several different oncogenes (including SV40T antigen and ras) that efficiently transform corresponding wild-type cells (Sell et al., Mol. Cell Biol., 14, 3604-12, 1994). Upregulation of components of the IGF axis has been described in various tumour cell lines and tissues, particularly tumours of the breast (Surmacz, Journal of Mammary Gland Biology & Neoplasia, 5, 95-105, 2000), prostate (Djavan et al, World J. Urol., 19, 225-233, 2001, and O'Brien et al, Urology, 58, 1-7, 2001), lung (Liao et al, Chinese J of Cancer, 25, 1238-1242, 2006, and Minuto et al Cancer Res., 46, 985-988, 1986) and colon (Guo et al, Gastroenterology, 102, 1101-1108, 1992). Conversely, IGF-IIR has been implicated as a tumour suppressor and is deleted in some cancers (DaCosta et al, Journal of Mammary Gland Biology & Neoplasia, 5, 85-94, 2000). There are a growing number of epidemiological studies linking increased circulating IGF (or increased ratio of IGF-1 to IGFBP3) with cancer risk (Yu and Rohan, J. Natl. Cancer Inst., 92, 1472-1489, 2000). Transgenic mouse models also implicate IGF signalling in the onset of tumour cell proliferation (Lamm and Christofori, Cancer Res. 58, 801-807, 1998, Foster et al, Cancer Metas. Rev., 17, 317-324, 1998, and DiGiovanni et al, Proc. Natl. Acad. Sci., 97, 3455-3460, 2000).

Several in vitro and in vivo strategies have provided the proof of principal that inhibition of IGF-1R signalling reverses the transformed phenotype and inhibits tumour cell growth. These include neutralizing antibodies (Kalebic et al Cancer Res., 54, 5531-5534, 1994), antisense oligonucleotides (Resnicoff et al, Cancer Res., 54, 2218-2222, 1994), triple-helix forming oligonucleotides (Rinninsland et al, Proc. Natl. Acad. Sci., 94, 5854-5859, 1997), antisense mRNA (Nakamura et al, Cancer Res., 60, 760-765, 2000) and dominant negative receptors (D'Ambrosio et al., Cancer Res., 56, 4013-4020, 1996). Antisense oligonucleotides have shown that inhibition of IGF-1R expression results in induction of apoptosis in cells in vivo (Resnicoff et al, Cancer Res., 55, 2463-2469, 1995) and have been taken into man (Resnicoff et al, Proc. Amer. Assoc. Cancer Res., 40 Abs 4816, 1999). However, none of these approaches is particularly attractive for the treatment of major solid tumour disease.

Since increased IGF signalling is implicated in the growth and survival of tumour cells, and blocking IGF-1R function can reverse this, inhibition of the IGF-1R tyrosine kinase domain is an appropriate therapy by which to treat cancer. In vitro and in vivo studies with the use of dominant-negative IGF-1R variants support this. In particular, a point mutation in the ATP binding site which blocks receptor tyrosine kinase activity has proved effective in preventing tumour cell growth (Kulik et al, Mol. Cell. Biol., 17, 1595-1606, 1997). Several pieces of evidence imply that normal cells are less susceptible to apoptosis caused by inhibition of IGF signalling, indicating that a therapeutic margin is possible with such treatment (Baserga, Trends Biotechnol., 14, 150-2, 1996).

There are few reports of selective IGF-1R tyrosine kinase inhibitors. Parrizas et al. described tyrphostins that had some efficacy in vitro and in vivo (Parrizas et al., Endocrinology, 138:1427-33 (1997)). These compounds were of modest potency and selectivity over the insulin receptor. Telik Inc. have described heteroaryl-aryl ureas which have selectivity over insulin receptors but potency against tumour cells in vitro is still modest (see WO 00/35455).

Pyrimidine derivatives substituted at the 2- and 4-positions by a substituted amino group having IGF-IR tyrosine kinase inhibitory activity are described in WO 03/048133. Compounds including an imidazopyridine or pyrazolopyridine substituent at the 4-position on the pyrimidine ring are not disclosed.

WO 02/50065 discloses that certain pyrazolyl-amino substituted pyrimidine derivatives have protein kinase inhibitory activity, especially as inhibitors of Aurora-2 and glycogen synthase kinase-3 (GSK-3), and are useful for treating diseases such as cancer, diabetes and Alzheimer's disease. The compounds disclosed have a substituted amino substituent at the 2-position of the pyrimidine ring but again there is no disclosure of compounds including an imidazopyridine or pyrazolopyridine substituent at the 4-position on the pyrimidine ring.

WO 01/60816 discloses that certain substituted pyrimidine derivatives have protein kinase inhibitory activity. There is no disclosure in WO 01/60816 of pyrimidine derivatives having an imidazo[1,2-a]pyridine or pyrazolo[2,3-a]pyridine substituent directly attached to the 4-position on the pyrimidine ring.

WO 01/14375 discloses imidazo[1,2-a]pyridine or pyrazolo[2,3-a]pyridine derivatives and their use in the inhibition of cell cycle kinases CDK2, CDK4 and CDK6. The derivatives may include a 2-anilinopyrimidine group, but there is no disclosure of such a derivative wherein the phenyl ring of the anilino substituent is itself substituted at an ortho-position on the phenyl ring and the pyrimidine ring includes a non-hydrogen substituent at the 5-position on the ring. Similar compounds are also disclosed in A. P. Thomas et al., Bioorg. Med. Chem. Lett. (2003), 13(8), 3021-3026, A. P. Thomas et al., Bioorg. Med. Chem. Lett. (2004), 14(9), 2249-2252 and A. P. Thomas et al., Bioorg. Med. Chem. Lett (2004), 14(9), 2245-2248.

WO 02/066480 discloses certain 2-anilino-pyrimidine compounds, which may include a imidazo[1,2-a]pyridine substituent, and their use in the inhibition of glycogen synthase kinase-3. There is no disclosure of such a compound wherein the phenyl ring of the anilino substituent is itself substituted at an ortho-position on the phenyl ring and the pyrimidine ring includes a non-hydrogen substituent at the 4-position on the ring.

Further imidazo[1,2-a]pyridinylpyrimidine derivatives are disclosed in WO 02/065979 and WO 2007/036732, but neither of these documents discloses a compound wherein the phenyl ring of the anilino substituent is itself substituted at an ortho-position on the phenyl ring and the pyrimidine ring includes a non-hydrogen substituent at the 5-position on the ring.

Whilst many of the compounds disclosed possess kinase activity and some may even display IGF-IR tyrosine kinase inhibitory activity, there is still a need for a compound which not only displays IGF-IR tyrosine kinase inhibitory activity but which possesses a balance of physical and biological properties. The present inventors have now found that certain pyrimidine compounds that contain an imidazopyridine or pyrazolopyridine substituent at the 4-position and a substituted anilino substituent at the 2-position on the pyrimidine ring possess potent anti-tumour activity. Without wishing to imply that the compounds of the present invention possess pharmacological activity only by virtue of an effect on a single biological process, it is believed that the compounds provide an anti-tumour effect by way of inhibition of IGF-1R tyrosine kinase activity. It is further believed that the compounds of the present invention selectively inhibit IGF-1R tyrosine kinase activity versus CDK2.

According to the present invention there is provided a compound of formula (I):

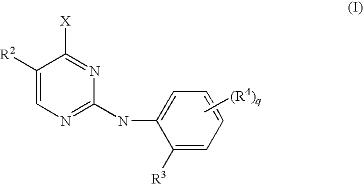

or a pharmaceutically acceptable salt thereof,
wherein:

X is selected from a group of formula Ia and Ib:

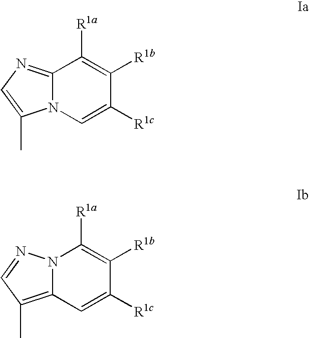

each R^{1a}, R^{1b} and R^{1c}, which may be the same or different, is selected from hydrogen, halogeno, cyano, (C1-C6)alkyl, (C1-C6)alkoxy, amino, (C1-C6)alkylamino and di-[(C1-C6)alkyl]amino, each of which groups within R^{1a}, R^{1b} and R^{1c} may be optionally substituted by one or more substituents independently selected from hydroxy, halogeno, cyano, (C1-C6)alkyl, (C1-C6)alkoxy, amino, (C1-C6)alkylamino, di-[(C1-C6)alkyl]amino, —N(R″)C(O)R′ wherein R′ is selected from hydrogen, (C1-C6)alkyl and (C1-C6)alkoxy and R″ is selected from hydrogen and (C1-C6)alkyl, and a saturated monocyclic 4-, 5-, 6-, 7- or 8-membered ring optionally comprising one or more heteroatoms independently selected from nitrogen, oxygen and sulfur;

R² is selected from halogeno, cyano, trifluoromethyl, cyclopropyl, (C1-C3)alkyl and (C1-C3)alkoxy;

R³ is selected from hydroxy, cyano, halogeno, (C1-C6)alkyl and (C1-C6)alkoxy, each of which groups within R³ may be optionally substituted by one or more substituents independently selected from hydroxy, halogeno, cyano and (C1-C6)alkoxy;

q represents 0, 1, 2, 3 or 4 (especially 1, 2, 3 or 4);

each R⁴, which may be the same or different, is selected from hydroxy, cyano, halogeno, formyl, carboxy, (C1-C6)alkyl, (C2-C6)alkenyl, (C2-C6)alkynyl, (C1-C6)alkoxy, (C2-C6)alkanoyl, (C1-C6)alkoxycarbonyl, (C3-C8)cycloalkyl, (C3-C8)cycloalkylcarbonyl, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (C1-C6)alkoxyamino, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, sulfamoyl, (C1-C6)alkylsulfamoyl, di-[(C1-C6)alkyl]sulfamoyl, —S(O)ₘR′ wherein R′ is as defined above and m represents 0, 1 or 2, —N(R″)C(O)R′ wherein R′ and R″ are each as defined above, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

or two R⁴ groups on adjacent carbon atoms of the phenyl ring, together with the carbon atoms to which they are attached, form a saturated or unsaturated monocyclic 5- or 6-membered heterocyclic ring comprising at least one ring heteroatom selected from nitrogen, oxygen and sulfur,

each of which groups or rings within R⁴ may be optionally substituted by one or more substituents independently selected from hydroxy, halogeno, cyano, formyl, carboxy, (C1-C6)alkyl, (C3-C8)cycloalkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, (C1-C6)alkylthio, sulfamoyl, (C1-C6)alkylsulfamoyl, di-[(C1-C6)alkyl]sulfamoyl, —S(O)ₘR′ wherein R′ and m are each as defined above, —N(R″)C(O)R′ wherein R′ and R″ are each as defined above, and —X-Q wherein X and Q are each as defined above, any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ and m are each as defined above, —N(R″)C(O)R′ wherein R′ and R″ are each as defined above, and —X-Q wherein X and Q are each as defined above;

and wherein any saturated monocyclic ring optionally bears 1 or 2 oxo or thioxo substituents.

In this specification, unless otherwise indicated, the term “alkyl” when used alone or in combination, includes both straight chain and branched chain alkyl groups, such as propyl, isopropyl and tert-butyl. However, references to individual alkyl groups such as “propyl” are specific for the straight-chain version only and references to individual branched-chain alkyl groups such as “isopropyl” are specific for the branched-chain version only. A (C1-C6)alkyl group has from one to six carbon atoms including methyl, ethyl, n-propyl, isopropyl, tert-butyl, n-pentyl, n-hexyl and the like. References to “(C1-C3)alkyl” will be understood accordingly to mean a straight or branched chain alkyl moiety having from one to three carbon atoms.

An analogous convention applies to other generic terms, for example, the terms “(C1-C6)alkoxy” and “(C1-C3)alkoxy”, when used alone or in combination, will be understood to refer to straight or branched chain groups having from one to six, or from one to three, carbon atoms respectively and include such groups as methoxy, ethoxy, propoxy and isopropoxy.

A “(C2-C6)alkenyl” group includes both straight chain and branched chain alkenyl groups having from two to six carbon atoms, such as vinyl, isopropenyl, allyl and but-2-enyl. Similarly, a “(C2-C6)alkynyl” group includes both straight chain and branched chain alkynyl groups having from two to six carbon atoms, such as ethynyl, 2-propynyl and but-2-ynyl.

The term “(C3-C8)cycloalkyl”, when used alone or in combination, refers to a saturated alicyclic moiety having from three to eight carbon atoms and includes, for example, cyclopropyl, cyclobutyl, cyclopentyl, cyclohexyl and cycloheptyl. References to “(C3-C6)cycloalkyl” will be understood accordingly to mean a saturated alicyclic moiety having from three to six carbon atoms, representative examples of which are listed above.

As used herein, the term “halogeno” includes fluoro, chloro, bromo and iodo.

The term “optionally substituted” is used herein to indicate optional substitution by the group or groups specified at any suitable available position.

A “heteroatom” is a nitrogen, sulfur or oxygen atom. Where rings include nitrogen atoms, these may be substituted as necessary to fulfil the bonding requirements of nitrogen or they may be linked to the rest of the structure by way of the nitrogen atom. Nitrogen atoms may also be in the form of N-oxides. Sulfur atoms may be in the form of S, S(O) or SO₂.

Suitable values for the generic radicals referred to above include those set out below.

A suitable value for a substituent Q when it is a “saturated 4-, 5-, 6-, 7-, 8-, 9- or 10-membered heterocyclic ring comprising at least one ring heteroatom selected from nitrogen, oxygen and sulfur” is a monocyclic or bicyclic heterocyclic ring containing 4, 5, 6, 7, 8, 9 or 10 atoms of which at least one is a heteroatom selected from nitrogen, oxygen and sulfur. The heterocyclic ring suitably contains from one to four (for example, from one to three, or one or two) heteroatoms independently selected from nitrogen, oxygen and sulfur. Unless specified otherwise, the heterocyclic ring may be carbon or nitrogen linked. Examples of suitable saturated monocyclic heterocyclic rings include azetidinyl, dioxanyl, trioxanyl, oxepanyl, dithianyl, trithianyl, oxathianyl, thiomorpholinyl, pyrrolidinyl, piperidinyl, imidazolidinyl, morpholinyl, tetrahydrofuranyl, tetrahydropyranyl, piperazinyl, 1,4-diazepanyl, 1,4-thiazinyl and 1,4-oxazepanyl (particularly azetidinyl, pyrrolidinyl, piperidinyl, morpholinyl, piperazinyl, 1,4-diazepanyl, 1,4-thiazinyl and 1,4-oxazepanyl). Examples of suitable saturated bicyclic heterocyclic rings include 2,5-diazabicyclo[2.2.1]heptanyl and 9-oxa-3,7-diazabicyclo[3.3.1]nonane. A saturated heterocyclic ring that bears 1 or 2 oxo or thioxo substituents may, for example, be 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl.

A suitable value for a “saturated monocyclic 4-, 5-, 6-, 7- or 8-membered ring optionally comprising one or more heteroatoms independently selected from nitrogen, oxygen and sulfur” may be a carbocyclic ring (that is an alicyclic ring having ring carbon atoms only) containing 4, 5, 6, 7 or 8 ring carbon atoms or a heterocyclic ring containing 4, 5, 6, 7 or 8 ring atoms of which at least one is a heteroatom selected from nitrogen, oxygen and sulfur. When the ‘saturated monocyclic 4-, 5-, 6-, 7- or 8-membered ring optionally comprising one or more heteroatoms independently selected from nitrogen, oxygen and sulfur’ is a heterocyclic ring, the heterocyclic ring preferably contains from one to four, more preferably from one to three, even more preferably from one to two, heteroatoms independently selected from nitrogen, oxygen and sulfur. Unless specified otherwise, the heterocyclic ring may be carbon or nitrogen linked. Examples of suitable carbocyclic rings include cyclobutyl, cyclopentyl, cyclohexyl and cycloheptyl. Examples of suitable saturated monocyclic 4-, 5-, 6-, 7- or 8-membered heterocyclic rings are provided above.

Where reference is made to “two R⁴ groups on adjacent carbon atoms of the phenyl ring, together with the carbon atoms to which they are attached, forming a saturated or unsaturated monocyclic 5- or 6-membered heterocyclic ring comprising at least one ring heteroatom selected from nitrogen, oxygen and sulfur”, it will be understood that this refers to saturated or fully or partially unsaturated monocyclic rings containing five or six atoms of which at least one is a heteroatom selected from nitrogen, oxygen and sulfur. The ring may have alicyclic or aromatic properties. Examples of saturated monocyclic 5- or 6-membered heterocyclic rings are provided above. Examples of 5- or 6-membered heteroaromatic rings include pyridyl, imidazolyl, isoxazolyl, pyrazolyl, furyl, pyrazinyl, pyridazinyl, pyrimidinyl, pyrrolyl, thiazolyl, oxazolyl, oxadiazolyl, isothiazolyl, triazolyl, tetrazolyl or thienyl.

For the avoidance of any doubt, the nitrogen atom at the 2-position on the pyrimidine ring linking the phenyl substituent thereto carries a hydrogen atom, i.e. such that the linker is a —NH— group.

Suitable values for any of the substituents herein, for example the ‘R’ groups (R^{1a}, R^{1b}, R^{1c}, R², R³, R⁴, R′ and R″) or for various groups within a X or Q group include:
for halogeno: fluoro, chloro, bromo and iodo;
for (C1-C6)alkyl: methyl, ethyl, propyl, isopropyl, tert-butyl, n-pentyl and n-hexyl;
for (C2-C6)alkenyl: vinyl, isopropenyl, allyl and but-2-enyl;
for (C2-C6)alkynyl: ethynyl, 2-propynyl and but-2-ynyl;
for (C1-C6)alkoxy: methoxy, ethoxy, propoxy, isopropoxy and butoxy; for (C1-C6)alkylamino: methylamino, ethylamino, propylamino, isopropylamino and butylamino;
for di-[(C1-C6)alkyl]amino: dimethylamino, diethylamino, N-ethyl-N-methylamino and N,N-diisopropylamino;
for amino(C1-C6)alkyl: aminomethyl, aminoethyl, aminopropyl and aminobutyl;
for (C1-C6)alkylamino(C1-C6)alkyl: methylaminomethyl, methylaminoethyl, methylaminopropyl, ethylaminomethyl, ethylaminoethyl, propylaminomethyl, isopropylaminoethyl and butylaminomethyl;
for di-[(C1-C6)alkyl]amino(C1-C6)alkyl: dimethylaminomethyl, dimethylaminoethyl, dimethylaminobutyl, diethylaminomethyl, diethylaminoethyl, diethylaminopropyl, N-ethyl-N-methylaminomethyl, N-ethyl-N-methylaminomethyl and N,N-diisopropylaminoethyl;
for (C2-C6)alkanoyl: methylcarbonyl, ethylcarbonyl, propylcarbonyl and tert-butylcarbonyl;
for (C1-C6)alkoxycarbonyl: methoxycarbonyl, ethoxycarbonyl, propoxycarbonyl and tert-butoxycarbonyl;
for (C1-C6)alkylcarbamoyl: N-methylcarbamoyl, N-ethylcarbamoyl and N-propylcarbamoyl;
for di-[(C1-C6)alkyl]carbamoyl: N,N-dimethylcarbamoyl, N-ethyl-N-methylcarbamoyl and N,N-diethylcarbamoyl;
for carbamoyl(C1-C6)alkyl: carbamoylmethyl, carbamoylethyl and carbamoylpropyl;
for (C1-C6)alkylcarbamoyl(C1-C6)alkyl: N-methylcarbamoylmethyl, N-ethylcarbamoylmethyl, N-propylcarbamoylmethyl, N-methylcarbamoylethyl, N-ethylcarbamoylethyl and N-propylcarbamoylethyl;
for di-[(C1-C6)alkyl]carbamoyl(C1-C6)alkyl: N,N-dimethylcarbamoylmethyl, N-ethyl-N-methylcarbamoylmethyl, N,N-diethylcarbamoylmethyl, N,N-dimethylcarbamoylethyl, N-ethyl-N-methylcarbamoylethyl and N,N-diethylcarbamoylethyl;
for (C3-C8)cycloalkyl: cyclopropyl, cyclobutyl, cyclopentyl, cyclohexyl and cycloheptyl;
for (C3-C8)cycloalkylcarbonyl: cyclopropylcarbonyl, cyclobutylcarbonyl, cyclopentylcarbonyl, cyclohexylcarbonyl and cycloheptylcarbonyl;
for (C1-C6)alkoxyamino: methoxyamino, ethoxyamino, propoxyamino and butoxyamino;
for (C1-C6)alkylsulfamoyl: methylsulfamoyl and ethylsulfamoyl;
for di-[(C1-C6)alkyl]sulfamoyl: dimethylsulfamoyl; and
for hydroxy(C1-C4)alkyl: hydroxymethyl and 2-hydroxyethyl.

Where the compounds according to the invention contain one or more asymmetrically substituted carbon atoms, the invention includes all stereoisomers, including enantiomers and diastereomers, and mixtures including racemic mixtures thereof.

Thus, it is to be understood that, insofar as certain of the compounds of formula (I) defined above may exist in optically active or racemic forms by virtue of one or more asymmetric carbon atoms, the invention includes in its definition any such optically active or racemic form which possesses the above-mentioned activity. The present invention encompasses all such stereoisomers having activity as herein defined. It is further to be understood that in the names of chiral compounds (R,S) denotes any scalemic or racemic mixture while (R) and (S) denote the enantiomers. In the absence of (R,S), (R) or (S) in the name it is to be understood that the name refers to any scalemic or racemic mixture, wherein a scalemic mixture contains R and S enantiomers in any relative proportions and a racemic mixture contains R and S enantiomers in the ratio 50:50. The synthesis of optically active forms may be carried out by standard techniques of organic chemistry well known in the art, for example by synthesis from optically active starting materials or by resolution of a racemic form. Racemates may be separated into individual enantiomers using known procedures (see, for example, Advanced Organic Chemistry: 3rd Edition: author J March, p 104-107). A suitable procedure involves formation of diastereomeric derivatives by reaction of the racemic material with a chiral auxiliary, followed by separation, for example by chromatography, of the diastereomers and then cleavage of the auxiliary species. Similarly, the above-mentioned activity may be evaluated using the standard laboratory techniques referred to hereinafter.

It is to be understood that, insofar as certain of the compounds of formula (I) defined above may exist in tautomeric forms, the invention includes in its definition any such tautomeric form which possesses the above-mentioned activity. Thus, the invention relates to all tautomeric forms of the compounds of formula (I) which inhibit IGF-1R tyrosine kinase activity in a human or animal.

It is to be understood that certain compounds of formula (I) may exist in solvated as well as unsolvated forms such as, for example, hydrated forms. It is to be understood that the invention encompasses all such solvated forms which inhibit IGF-1R tyrosine kinase activity in a human or animal.

It is also to be understood that certain compounds of formula (I) may exhibit polymorphism, and that the invention encompasses all such forms which inhibit IGF-1R tyrosine kinase activity in a human or animal.

Thus, throughout the specification, where reference is made to the compound of formula (I), it is understood that the term compound includes isomers, mixtures of isomers, solvates, stereoisomers, and polymorphs that inhibit IGF-1R tyrosine kinase activity in a human or animal.

The present invention relates to the compound of formula (I) as herein defined as well as to salts thereof. Salts for use in pharmaceutical compositions will be pharmaceutically acceptable salts, but other salts may be useful in the production of the compound of formula (I) and their pharmaceutically acceptable salts. Pharmaceutically acceptable salts of the invention may, for example, include acid addition salts of compound of formula (I), as herein defined, wherein the compound of formula (I) is sufficiently basic to form such salts, and base salts of compound of formula (I), as herein defined, wherein the compound of formula (I) is sufficiently acidic to form such salts. Such acid addition salts include but are not limited to fumarate, methanesulfonate, hydrochloride, hydrobromide, citrate and maleate salts and salts formed with phosphoric and sulfuric acid, and also salts formed by sulfonic acids such as ethane sulfonic acid, ethane disulfonic acid, benzene sulfonic acid and toluene sulfonic acid. Such base salts include but are not limited to alkali metal salts for example sodium salts, alkaline earth metal salts for example calcium or magnesium salts, and organic amine salts for example triethylamine, morpholine, N-methylpiperidine, N-ethylpiperidine, procaine, dibenzylamine, N,N-dibenzylethylamine or amino acids for example lysine.

The addition salts may also include acetate, adipate, citrate, citrate, D,L-lactate, D,L-mandelate, fumarate, glutarate, glycolate, hippurate, hydrochloride, maleate, malate, malonate, napadysilate, phosphorate, sulphate, and also salts formed by sulfonic acids such as benzenesulfonic acid, ethanedisulfonic acid and toluenesulfonic acid, and also salts formed by saccharin.

According to an aspect of the present invention there is provided a compound of formula (I), or a pharmaceutically acceptable salt thereof, wherein X is a group of formula Ia as defined herein and R², R³, R⁴ and q are each as defined herein.

According to another aspect of the present invention there is provided a compound of formula (I), or a pharmaceutically acceptable salt thereof, wherein X is a group of formula Ib as defined herein and R², R³, R⁴ and q are each as defined herein.

In particular, in the compounds of formula (I), each R^{1a}, R^{1b} and R^{1c}, which may be the same or different, may be selected from hydrogen, halogeno, cyano, (C1-C3)alkyl, (C1-C3)alkoxy, amino, (C1-C3)alkylamino and di-[(C1-C3)alkyl]amino, each of which groups within R^{1a}, R^{1b} and R^{1c} may be optionally substituted by one or more substituents independently selected from hydroxy and halogeno (especially hydroxy).

More particularly, in the compounds of formula (I), each R^{1a}, R^{1b} and R^{1c}, which may be the same or different, may be selected from hydrogen, halogeno, cyano, (C1-C3)alkyl, (C1-C3)alkoxy and di-[(C1-C3)alkyl]amino, each of which groups within R^{1a}, R^{1b} and R^{1c} may be optionally substituted by one or more substituents independently selected from hydroxy and halogeno (especially hydroxy).

Even more particularly, in the compounds of formula (I), each R^{1a}, R^{1b} and R^{1c}, which may be the same or different, may be selected from hydrogen, halogeno, cyano, methyl, ethyl, methoxy and dimethylamino, each of which groups within R^{1a}, R^{1b} and R^{1c} may be optionally substituted by one or more substituents independently selected from hydroxy and halogeno (especially hydroxy).

In one aspect, in the compounds of formula (I), one or two (especially one) of the groups R^{1a}, R^{1b} and R^{1c}, which may be the same or different, may be selected from halogeno, cyano, (C1-C6)alkyl, (C1-C6)alkoxy, amino, (C1-C6)alkylamino and di-[(C1-C6)alkyl]amino, each of which groups within R^{1a}, R^{1b} and R^{1c} may be optionally substituted by one or more substituents independently selected from hydroxy, halogeno, cyano, (C1-C6)alkyl, (C1-C6)alkoxy, amino, (C1-C6)alkylamino, di-[(C1-C6)alkyl]amino, —N(R″)C(O)R′ wherein R′ is selected from hydrogen, (C1-C6)alkyl and (C1-C6)alkoxy and R″ is selected from hydrogen and (C1-C6)alkyl, and a saturated monocyclic 4-, 5-, 6-, 7- or 8-membered ring optionally comprising one or more heteroatoms independently selected from nitrogen, oxygen and sulfur, and the remaining one or two (especially two) groups R^{1a}, R^{1b} and R^{1c} may be hydrogen.

In another aspect, in the compounds of formula (I), one or two (especially one) of the groups R^{1a}, R^{1b} and R^{1c}, which may be the same or different, may be selected from halogeno, cyano, (C1-C3)alkyl, (C1-C3)alkoxy and di-[(C1-C3)alkyl]amino, each of which groups within R^{1a}, R^{1b} and R^{1c} may be optionally substituted by one or more substituents independently selected from hydroxy and halogeno (especially hydroxy) and the remaining one or two (especially two) groups R^{1a}, R^{1b} and R^{1c} may be hydrogen.

In another aspect, in the compounds of formula (I), one or two (especially one) of the groups R^{1a}, R^{1b} and R^{1c}, which may be the same or different, may be selected from halogeno, cyano, (C1-C3)alkyl, (C1-C3)alkoxy, amino, (C1-C3)alkylamino and di-[(C1-C3)alkyl]amino, each of which groups within R^{1a}, R^{1b} and R^{1c} may be optionally substituted by one or more substituents independently selected from hydroxy and halogeno (especially hydroxy) and the remaining one or two (especially two) groups R^{1a}, R^{1b} and R^{1c} may be hydrogen.

In another aspect, in the compounds of formula (I), one or two (especially one) of the groups R^{1a}, R^{1b} and R^{1c}, which may be the same or different, may be selected from halogeno, cyano, methyl, ethyl, methoxy and dimethylamino, each of which groups within R^{1a}, R^{1b} and R^{1c} may be optionally substituted by one or more substituents independently selected from hydroxy and halogeno (especially hydroxy) and the remaining one or two (especially two) groups R^{1a}, R^{1b} and R^{1c} may be hydrogen.

In another aspect, in the compounds of formula (I), the groups R^{1a}, R^{1b} and R^{1c} all represent hydrogen. In yet another aspect, in the compounds of formula (I), the groups R^{1a}, R^{1b} and R^{1c} all represent hydrogen and X represents a group of formula Ia.

In particular, in the compounds of formula (I), R² may be selected from halogeno, cyano, trifluoromethyl, (C1-C3)alkyl and (C1-C3)alkoxy, such as selected from chloro, fluoro, bromo, cyano, trifluoromethyl, methyl and methoxy. In one particular aspect, R² may be halogeno, for example chloro, fluoro or bromo, especially chloro or fluoro. In one particular aspect, R² is chloro. In another particular aspect, R² is fluoro. In another particular aspect, R² is methyl.

In particular, in the compounds of formula (I), R³ may be selected from halogeno, (C1-C6)alkyl and (C1-C6)alkoxy (especially from (C1-C6)alkyl and (C1-C6)alkoxy), each of which groups within R³ may be optionally substituted by one or more substituents independently selected from hydroxy, halogeno, cyano and (C1-C6)alkoxy.

More particularly, in the compounds of formula (I), R³ may be selected from (C1-C6)alkyl and (C1-C6)alkoxy (especially (C1-C6)alkoxy), each of which groups within R³ may be optionally substituted by one or more substituents independently selected from hydroxy, halogeno, cyano and (C1-C6)alkoxy. Yet more particularly, in the compounds of formula (I), R³ may be selected from (C1-C3)alkyl and (C1-C3)alkoxy (such as methyl and methoxy). For example, in the compounds of formula (I), R³ may be (C1-C3)alkoxy, most particularly methoxy.

In one particular aspect, in the compounds of formula (I), R² may be chloro, fluoro, bromo, cyano, trifluoromethyl, methyl or methoxy and R³ may be (C1-C3)alkoxy (for example methoxy).

In another particular aspect, in the compounds of formula (I), R² may be halogeno (for example chloro, fluoro or bromo, especially chloro or fluoro) and R³ may be (C1-C3)alkoxy (for example methoxy).

In another particular aspect, in the compounds of formula (I), R² may be halogeno (for example chloro, fluoro or bromo, especially chloro or fluoro) or methyl and R³ may be (C1-C3)alkoxy (for example methoxy).

In particular, in the compounds of formula (I), q may be 1, 2 or 3, especially 1 or 2, more especially 1.

In particular, in the compounds of formula (I), each R⁴, which may be the same or different, may be selected from hydroxy, cyano, halogeno, formyl, carboxy, (C1-C6)alkyl, (C2-C6)alkenyl, (C2-C6)alkynyl, (C1-C6)alkoxy, (C2-C6)alkanoyl, (C1-C6)alkoxycarbonyl, (C3-C8)cycloalkyl, (C3-C8)cycloalkylcarbonyl, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

each of which groups or rings within R⁴ may be optionally substituted by one or more substituents independently selected from hydroxy, halogeno, cyano, carboxy, (C1-C6)alkyl, (C3-C8)cyclo alkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, (C1-C6)alkylthio, sulfamoyl, (C1-C6)alkylsulfamoyl, di-[(C1-C6)alkyl]sulfamoyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above (especially X is —C(O)—), any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above;

and wherein any saturated monocyclic ring optionally bears 1 or 2 oxo substituents.

More particularly, in the compounds of formula (I), each R⁴, which may be the same or different, may be selected from hydroxy, cyano, halogeno, formyl, carboxy, (C1-C6)alkyl, (C2-C6)alkenyl, (C2-C6)alkynyl, (C1-C6)alkoxy, (C2-C6)alkanoyl, (C1-C6)alkoxycarbonyl, (C3-C8)cycloalkyl, (C3-C8)cycloalkylcarbonyl, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

each of which groups or rings within R⁴ may be optionally substituted by one or more substituents independently selected from hydroxy, carboxy, (C1-C6)alkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above (especially X is —C(O)—), any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above;

and wherein any saturated monocyclic ring optionally bears 1 or 2 oxo substituents.

Even more particularly, in the compounds of formula (I), each R⁴, which may be the same or different, may be selected from (C1-C6)alkyl, (C1-C6)alkoxy and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

each of which groups or rings within R⁴ may be optionally substituted by one or more substituents independently selected from hydroxy, carboxy, (C1-C6)alkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above (especially X is —C(O)—), any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above;

and wherein any saturated monocyclic ring optionally bears 1 or 2 oxo substituents.

Yet more particularly, in the compounds of formula (I), each R⁴, which may be the same or different, may be selected from (C1-C6)alkyl, (C1-C6)alkoxy and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

each of which groups or rings within R⁴ may be optionally substituted by one or more substituents independently selected from hydroxy, carboxy, (C1-C6)alkyl, (C3-C8)cycloalkylcarbonyl, (C1-C6)alkoxy, amino, di-[(C1-C6)alkyl]amino, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, di-[(C1-C6)alkyl]carbamoyl, (C1-C6)alkylcarbamoyl(C1-C6)alkyl, di-[(C1-C6)alkyl]carbamoyl(C1-C6)alkyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above (especially X is —C(O)—), any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above;

and wherein any saturated monocyclic ring optionally bears 1 or 2 oxo substituents.

In particular, in the compounds of formula (I), each R⁴, which may be the same or different, is selected from halogeno, formyl, carboxy, (C1-C6)alkyl, (C2-C6)alkenyl, (C2-C6)alkynyl, (C1-C6)alkoxy, amino(C1-C6)alkyl, (C1-C6)alkylamino(C1-C6)alkyl, di-[(C1-C6)alkyl]amino(C1-C6)alkyl, carbamoyl, (C1-C6)alkylcarbamoyl, di-[(C1-C6)alkyl]carbamoyl, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

each of which groups or rings within R⁴ may be optionally substituted by one or more substituents independently selected from hydroxy, carboxy, (C1-C6)alkyl, (C3-C8)cycloalkyl, (C3-C8)cycloalkylcarbonyl, (C1-C6)alkoxy, amino, (C1-C6)alkylamino, di-[(C1-C6)alkyl]amino, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, sulfamoyl, (C1-C6)alkylsulfamoyl, di-[(C1-C6)alkyl]sulfamoyl, —S(O)ₘR′ wherein R′ and m are each as defined above, —N(R″)C(O)R′ wherein R′ and R″ are each as defined above, and —X-Q wherein X and Q are each as defined above (especially X is —C(O)—), any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ and m are each as defined above, —N(R″)C(O)R′ wherein R′ and R″ are each as defined above, and —X-Q wherein X and Q are each as defined above (especially X is a direct bond or —C(O)—);

and wherein any saturated monocyclic ring optionally bears 1 or 2 oxo substituents.

More particularly, in the compounds of formula (I), each R⁴, which may be the same or different, is selected from halogeno, formyl, carboxy, (C1-C6)alkyl, (C1-C6)alkoxy, (C1-C6)alkylamino(C1-C6)alkyl, di-[(C1-C6)alkyl]amino(C1-C6)alkyl, di-[(C1-C6)alkyl]carbamoyl, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

each of which groups or rings within R⁴ may be optionally substituted by one or more substituents independently selected from hydroxy, carboxy, (C1-C6)alkyl, (C3-C8)cycloalkylcarbonyl, (C1-C6)alkoxy, amino, di-[(C1-C6)alkyl]amino, (2-6C)alkanoyl, (C1-C6)alkoxycarbonyl, carbamoyl, (C1-C6)alkylcarbamoyl, di-[(C1-C6)alkyl]carbamoyl, (C1-C6)alkylcarbamoyl(C1-C6)alkyl, di-[(C1-C6)alkyl]carbamoyl(C1-C6)alkyl, di-[(C1-C6)alkyl]sulfamoyl, —S(O)ₘR′ wherein R′ and m are each as defined above, —N(R″)C(O)R′ wherein R′ and R″ are each as defined above, and —X-Q wherein X and Q are each as defined above (especially X is —C(O)—), any of which substituents may be optionally substituted by one or more further substituents independently selected from (C1-C4)alkoxy, hydroxy, cyano, hydroxy(C1-C4)alkyl, amino, (C1-C6)alkylamino, carbamoyl, —S(O)ₘR′ wherein R′ and m are each as defined above, —N(R″)C(O)R′ wherein R′ and R″ are each as defined above, and —X-Q wherein X and Q are each as defined above (especially X is a direct bond or —C(O)—);

and wherein any saturated monocyclic ring optionally bears 1 or 2 oxo substituents.

More particularly, in the compounds of formula (I), each R⁴, which may be the same or different, is selected from halogeno, (C1-C6)alkyl, (C1-C6)alkoxy, (C1-C6)alkylamino(C1-C6)alkyl, di-[(C1-C6)alkyl]amino(C1-C6)alkyl, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

each of which groups or rings within R⁴ may be optionally substituted by one or more substituents independently selected from hydroxy, (C1-C6)alkyl, (C3-C8)cycloalkylcarbonyl, (C1-C6)alkoxy, amino, di-[(C1-C6)alkyl]amino, (2-6C)alkanoyl, (C1-C6)alkoxycarbonyl, carbamoyl, di-[(C1-C6)alkyl]carbamoyl, (C1-C6)alkylcarbamoyl(C1-C6)alkyl, di-[(C1-C6)alkyl]carbamoyl(C1-C6)alkyl, di-[(C1-C6)alkyl]sulfamoyl, —S(O)ₘR′ wherein R′ and m are each as defined above, —N(R″)C(O)R′ wherein R′ and R″ are each as defined above, and —X-Q wherein X and Q are each as defined above (especially X is —C(O)—), any of which substituents may be optionally substituted by one or more further substituents independently selected from (C1-C4)alkoxy, hydroxy, cyano, hydroxy(C1-C4)alkyl, amino, (C1-C6)alkylamino, —S(O)ₘR′ wherein R′ and m are each as defined above, and —X-Q wherein X and Q are each as defined above (especially X is a direct bond or —C(O)—);

and wherein any saturated monocyclic ring optionally bears 1 or 2 oxo substituents.

In one aspect, in the compounds of formula (I), each R⁴, which may be the same or different, may be selected from —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

each of which groups or rings within R⁴ may be optionally substituted by one or more substituents independently selected from hydroxy, (C1-C6)alkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above (especially X is —C(O)—), any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above;

and wherein any saturated monocyclic ring optionally bears 1 or 2 oxo substituents.

In another aspect, in the compounds of formula (I), each R⁴, which may be the same or different, may be selected from —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

each of which groups or rings within R⁴ may be optionally substituted by one or more substituents independently selected from (C1-C6)alkyl and (2-6C)alkanoyl, any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ is selected from hydrogen and (C1-C6)alkyl and m represents 2, —N(R″)C(O)R′ wherein R′ is selected from hydrogen and (C1-C6)alkyl and R″ is selected from hydrogen and (C1-C6)alkyl, and —X-Q wherein X and Q are each as defined above;

and wherein any saturated monocyclic ring optionally bears 1 or 2 oxo substituents.

In another aspect, in the compounds of formula (I), each R⁴, which may be the same or different, may be selected from —X-Q wherein X is a direct bond and Q represents a saturated 4-, 5-, 6-, 7-, 8-, 9- or 10-membered heterocyclic ring comprising at least one ring heteroatom selected from nitrogen, oxygen and sulfur,

each of which rings within R⁴ may be optionally substituted by one or more substituents independently selected from (C1-C6)alkyl and (2-6C)alkanoyl, any of which substituents may be optionally substituted by one or more further substituents independently selected from (C1-C4)alkyl, (C1-C4)alkoxy, hydroxy and hydroxy(C1-C4)alkyl,

and wherein any saturated monocyclic ring optionally bears 1 or 2 oxo substituents.

In another aspect, in the compounds of formula (I), each R⁴, which may be the same or different, may be selected from —X-Q wherein X is a direct bond and Q represents a saturated 4-, 5-, 6-, 7-, 8-, 9- or 10-membered heterocyclic ring comprising at least one ring nitrogen atom and optionally comprising a further one or two ring heteroatoms selected from nitrogen, oxygen and sulfur,

each of which rings within R⁴ may be optionally substituted by one or more substituents independently selected from (C1-C6)alkyl and (2-6C)alkanoyl, any of which substituents may be optionally substituted by one or more further substituents independently selected from (C1-C4)alkyl, (C1-C4)alkoxy, (C1-C4)alkylamino, di-[(C1-C4)alkyl]amino, hydroxy and hydroxy(C1-C4)alkyl,

and wherein any saturated monocyclic ring optionally bears 1 or 2 oxo substituents.

Suitable values for R⁴ include, for example, fluoro, methyl, methoxy, (2-hydroxyethyl-methylcarbamoyl), 2,5-diazabicyclo[2.2.1]heptane-5-carbonyl, 2-methyl-2,5-diazabicyclo[2.2.1]heptane-5-carbonyl, [4-(2-hydroxyethyl)piperazine-1-carbonyl], piperazine-1-carbonyl, morpholine-4-carbonyl, [1-[4-(2-hydroxyethyl)piperazin-1-yl]-1-oxopropan-2-yl], (1-oxo-1-piperazin-1-ylpropan-2-yl), (1-morpholin-4-yl-1-oxopropan-2-yl), 1,3-dihydroxypropan-2-yl, (5-oxopyrrolidin-3-yl), [5-(methylcarbamoyl)pyrrolidin-3-yl], (1-acetylpiperidin-2-yl), (1-acetylpiperidin-3-yl), (1-acetyl-2-methylpiperidin-4-yl), [1-(2-hydroxypropanoyl)piperidin-4-yl], (1-acetylpiperidin-4-yl), [1-(2-methoxypropanoyl)piperidin-4-yl], [1-(dimethylcarbamoyl)piperidin-4-yl], [1-(2-hydroxyethyl-methylcarbamoyl)piperidin-4-yl], (2-methylpiperidin-4-yl), piperidin-4-yl, oxan-4-yl, [2-(3-hydroxypyrrolidin-1-yl)-2-oxoethyl], [2-[4-(2-hydroxyethyl)piperazin-1-yl]-2-oxoethyl], (2-oxo-2-piperazin-1-ylethyl), (2-morpholin-4-yl-2-oxoethyl), [2-oxo-2-(pyrrolidin-3-ylamino)ethyl], (2-hydroxyethyl-methylamino)methyl, (2-methoxyethyl-methylamino)methyl, (1-hydroxypropan-2-ylamino)methyl, (3-hydroxyazetidin-1-yl)methyl, (3-hydroxypyrrolidin-1-yl)methyl, (4-carbamoylpiperidin-1-yl)methyl, (4-hydroxypiperidin-1-yl)methyl, (3-hydroxypiperidin-1-yl)methyl, azetidin-1-ylmethyl, (4-methyl-1,4-diazepan-1-yl)methyl, 1,4-oxazepan-4-ylmethyl, (4-methylpiperazin-1-yl)methyl, (4-acetylpiperazin-1-yl)methyl, [4-(2-hydroxyethyl)piperazin-1-yl]methyl, morpholin-4-ylmethyl, (3-hydroxypropylamino)methyl, (3-methoxypropylamino)methyl, (2-hydroxyethylamino)methyl, (4-acetyl-2,5-dimethylpiperazin-1-yl), (4-acetyl-3,5-dimethylpiperazin-1-yl), (3,5-dimethylpiperazin-1-yl), (3-aminopyrrolidin-1-yl), (3-dimethylaminopyrrolidin-1-yl), (3-acetamidopyrrolidin-1-yl), (3-hydroxypyrrolidin-1-yl), (4-hydroxypiperidin-1-yl), (4-methoxypiperidin-1-yl), pyrrolidin-1-yl, [2-(morpholine-4-carbonyl)pyrrolidin-1-yl], (4-acetyl-1,4-diazepan-1-yl), 1,4-oxazepan-4-yl, (4-acetyl-3-methylpiperazin-1-yl), [4-acetyl-3-(hydroxymethyl)piperazin-1-yl], (4-acetyl-6-hydroxy-1,4-diazepan-1-yl), (4-methylpiperazin-1-yl), [4-(2-amino-3-methylbutanoyl)piperazin-1-yl], [4-(2-aminopropanoyl)piperazin-1-yl], [4-(2-hydroxypropanoyl)piperazin-1-yl], [4-(2,4-diamino-4-oxobutanoyl)piperazin-1-yl], [4-(2-amino-3-hydroxypropanoyl)piperazin-1-yl], [4-(2,3-dihydroxypropanoyl)piperazin-1-yl], [4-(4-hydroxypyrrolidine-2-carbonyl)piperazin-1-yl], [4-(5-oxopyrrolidine-2-carbonyl)piperazin-1-yl], [4-(piperidine-2-carbonyl)piperazin-1-yl], [4-(pyrrolidine-2-carbonyl)piperazin-1-yl], (4-acetylpiperazin-1-yl), [4-(2-methoxypropanoyl)piperazin-1-yl], [4-(2-amino-4-hydroxybutanoyl)piperazin-1-yl], [4-(2-hydroxy-2-methylpropanoyl)piperazin-1-yl], [4-(1-hydroxycyclopropanecarbonyl)piperazin-1-yl], [4-(5-oxopyrrolidine-2-carbonyl)piperazin-1-yl], (4-propanoylpiperazin-1-yl), [4-(2-aminoacetyl)piperazin-1-yl], [4-(2-methylaminoacetyl)piperazin-1-yl], [4-(2-acetamidoacetyl)piperazin-1-yl], [4-(2-ethylaminoacetyl)piperazin-1-yl], [4-(2-hydroxyacetyl)piperazin-1-yl], [4-(2-methoxyacetyl)piperazin-1-yl], (4-carbamoylpiperazin-1-yl), [4-(dimethylcarbamoyl)piperazin-1-yl], [4-(2-hydroxyethyl-methylcarbamoyl)piperazin-1-yl], [4-(piperazine-1-carbonyl)piperazin-1-yl], [4-(morpholine-4-carbonyl)piperazin-1-yl], (4-methoxycarbonylpiperazin-1-yl), [4-(2-methoxyethoxycarbonyl)piperazin-1-yl], [4-(2-hydroxypropyl)piperazin-1-yl], (4-ethylpiperazin-1-yl), [4-(2,3-dihydroxypropyl)piperazin-1-yl], [4-(2-dimethylamino-2-oxo ethyl)piperazin-1-yl], [4-[2-(2-hydroxyethyl-methylamino)-2-oxoethyl]piperazin-1-yl], [4-(2-morpholin-4-yl-2-oxoethyl)piperazin-1-yl], [4-(2-methylamino-2-oxoethyl)piperazin-1-yl], [4-(2-hydroxy-2-methylpropyl)piperazin-1-yl], [4-(2-cyano ethyl)piperazin-1-yl], [4-(2-hydroxyethyl)piperazin-1-yl], [4-(2-methoxyethyl)piperazin-1-yl], [4-(2-methylsulfonylethyl)piperazin-1-yl], (4-methylsulfonylpiperazin-1-yl), [4-(dimethylsulfamoyl)piperazin-1-yl], (3-oxopiperazin-1-yl), [3-(dimethylcarbamoyl)piperazin-1-yl], [4-(cyclopropanecarbonyl)piperazin-1-yl], [4-(pyrrolidine-2-carbonyl)piperazin-1-yl], piperazin-1-yl, morpholin-4-yl, (1,1-dioxo-1,4-thiazinan-4-yl), [1-(2-hydroxypropanoyl)pyrrolidin-3-yl]oxy, [1-(2-hydroxypropanoyl)pyrrolidin-3-yl]oxy, (1-acetylpyrrolidin-3-yl)oxy, (1-methylsulfonylpyrrolidin-3-yl)oxy, pyrrolidin-3-yloxy, pyrrolidin-2-ylmethoxy, (1-acetylpyrrolidin-2-yl)methoxy, (1-methylpyrrolidin-3-yl)oxy, [1-(2-hydroxypropanoyl)piperidin-4-yl]oxy, (1-acetylpiperidin-4-yl)oxy, [1-(2-hydroxypropanoyl)piperidin-4-yl]oxy, (1-methylsulfonylpiperidin-4-yl)oxy, piperidin-4-yloxy, [1-(2-hydroxypropanoyl)azetidin-3-yl]oxy, [1-(2-methoxypropanoyl)azetidin-3-yl]oxy, (1-acetylazetidin-3-yl)oxy, [1-(1-hydroxycyclopropanecarbonyl)azetidin-3-yl]oxy, [1-(2-hydroxyacetyl)azetidin-3-yl]oxy, [1-(dimethylcarbamoyl)azetidin-3-yl]oxy, [1-(2-hydroxyethyl-methylcarbamoyl)azetidin-3-yl]oxy, [1-(2-hydroxypropyl)azetidin-3-yl]oxy, [1-(2-hydroxy-2-methylpropyl)azetidin-3-yl]oxy, [1-(2-hydroxyethyl)azetidin-3-yl]oxy, azetidin-3-yloxy, 2-[4-(2-methoxyethyl)piperazin-1-yl]ethoxy, formyl, carboxy and carboxymethyl.

Suitable values for R⁴ also include, for example, fluoro, methyl, methoxy, (2-hydroxyethyl-methylcarbamoyl), 2,5-diazabicyclo[2.2.1]heptane-5-carbonyl, 2-methyl-2,5-diazabicyclo[2.2.1]heptane-5-carbonyl, [4-(2-hydroxyethyl)piperazine-1-carbonyl], piperazine-1-carbonyl, morpholine-4-carbonyl, [1-[4-(2-hydroxyethyl)piperazin-1-yl]-1-oxopropan-2-yl], (1-oxo-1-piperazin-1-ylpropan-2-yl), (1-morpholin-4-yl-1-oxopropan-2-yl), 1,3-dihydroxypropan-2-yl, (5-oxopyrrolidin-3-yl), [5-(methylcarbamoyl)pyrrolidin-3-yl], (1-acetylpiperidin-2-yl), (1-acetylpiperidin-3-yl), (1-acetyl-2-methylpiperidin-4-yl), [1-(2-hydroxypropanoyl)piperidin-4-yl], (1-acetylpiperidin-4-yl), [1-(2-methoxypropanoyl)piperidin-4-yl], [1-(dimethylcarbamoyl)piperidin-4-yl], [1-(2-hydroxyethyl-methylcarbamoyl)piperidin-4-yl], (2-methylpiperidin-4-yl), piperidin-4-yl, oxan-4-yl, [2-(3-hydroxypyrrolidin-1-yl)-2-oxo ethyl], [2-[4-(2-hydroxyethyl)piperazin-1-yl]-2-oxo ethyl], (2-oxo-2-piperazin-1-ylethyl), (2-morpholin-4-yl-2-oxo ethyl), [2-oxo-2-(pyrrolidin-3-ylamino)ethyl], (2-hydroxyethyl-methylamino)methyl, (2-methoxyethyl-methylamino)methyl, (1-hydroxypropan-2-ylamino)methyl, (3-hydroxyazetidin-1-yl)methyl, (3-hydroxypyrrolidin-1-yl)methyl, (4-carbamoylpiperidin-1-yl)methyl, (4-hydroxypiperidin-1-yl)methyl, (3-hydroxypiperidin-1-yl)methyl, azetidin-1-ylmethyl, (4-methyl-1,4-diazepan-1-yl)methyl, 1,4-oxazepan-4-ylmethyl, (4-methylpiperazin-1-yl)methyl, (4-acetylpiperazin-1-yl)methyl, [4-(2-hydroxyethyl)piperazin-1-yl]methyl, morpholin-4-ylmethyl, (3-hydroxypropylamino)methyl, (3-methoxypropylamino)methyl, (2-hydroxyethylamino)methyl, (4-acetyl-2,5-dimethylpiperazin-1-yl), (4-acetyl-3,5-dimethylpiperazin-1-yl), (3,5-dimethylpiperazin-1-yl), (3-aminopyrrolidin-1-yl), (3-dimethylaminopyrrolidin-1-yl), (3-acetamidopyrrolidin-1-yl), (3-hydroxypyrrolidin-1-yl), (4-hydroxypiperidin-1-yl), (4-methoxypiperidin-1-yl), pyrrolidin-1-yl, [2-(morpholine-4-carbonyl)pyrrolidin-1-yl], (4-acetyl-1,4-diazepan-1-yl), 1,4-oxazepan-4-yl, (4-acetyl-3-methylpiperazin-1-yl), [4-acetyl-3-(hydroxymethyl)piperazin-1-yl], (4-acetyl-6-hydroxy-1,4-diazepan-1-yl), (4-methylpiperazin-1-yl), [4-(2-amino-3-methylbutanoyl)piperazin-1-yl], [4-(2-aminopropanoyl)piperazin-1-yl], [4-(2-hydroxypropanoyl)piperazin-1-yl], [4-(2,4-diamino-4-oxobutanoyl)piperazin-1-yl], [4-(2-amino-3-hydroxypropanoyl)piperazin-1-yl], [4-(2,3-dihydroxypropanoyl)piperazin-1-yl], [4-(4-hydroxypyrrolidine-2-carbonyl)piperazin-1-yl], [4-(5-oxopyrrolidine-2-carbonyl)piperazin-1-yl], [4-(piperidine-2-carbonyl)piperazin-1-yl], [4-(pyrrolidine-2-carbonyl)piperazin-1-yl], (4-acetylpiperazin-1-yl), [4-(2-methoxypropanoyl)piperazin-1-yl], [4-(2-amino-4-hydroxybutanoyl)piperazin-1-yl], [4-(2-hydroxy-2-methylpropanoyl)piperazin-1-yl], [4-(1-hydroxycyclopropanecarbonyl)piperazin-1-yl], [4-(5-oxopyrrolidine-2-carbonyl)piperazin-1-yl], (4-propanoylpiperazin-1-yl), [4-(2-aminoacetyl)piperazin-1-yl], [4-(2-methylaminoacetyl)piperazin-1-yl], [4-(2-acetamidoacetyl)piperazin-1-yl], [4-(2-ethylaminoacetyl)piperazin-1-yl], [4-(2-hydroxyacetyl)piperazin-1-yl], [4-(2-methoxyacetyl)piperazin-1-yl], (4-carbamoylpiperazin-1-yl), [4-(dimethylcarbamoyl)piperazin-1-yl], [4-(2-hydroxyethyl-methylcarbamoyl)piperazin-1-yl], [4-(piperazine-1-carbonyl)piperazin-1-yl], [4-(morpholine-4-carbonyl)piperazin-1-yl], (4-methoxycarbonylpiperazin-1-yl), [4-(2-methoxyethoxycarbonyl)piperazin-1-yl], [4-(2-hydroxypropyl)piperazin-1-yl], (4-ethylpiperazin-1-yl), [4-(2,3-dihydroxypropyl)piperazin-1-yl], [4-(2-dimethylamino-2-oxo ethyl)piperazin-1-yl], [4-[2-(2-hydroxyethyl-methylamino)-2-oxoethyl]piperazin-1-yl], [4-(2-morpholin-4-yl-2-oxoethyl)piperazin-1-yl], [4-(2-methylamino-2-oxoethyl)piperazin-1-yl], [4-(2-hydroxy-2-methylpropyl)piperazin-1-yl], [4-(2-cyanoethyl)piperazin-1-yl], [4-(2-hydroxyethyl)piperazin-1-yl], [4-(2-methoxyethyl)piperazin-1-yl], [4-(2-methylsulfonylethyl)piperazin-1-yl], (4-methylsulfonylpiperazin-1-yl), [4-(dimethylsulfamoyl)piperazin-1-yl], (3-oxopiperazin-1-yl), [3-(dimethylcarbamoyl)piperazin-1-yl], [4-(cyclopropanecarbonyl)piperazin-1-yl], [4-(pyrrolidine-2-carbonyl)piperazin-1-yl], piperazin-1-yl, morpholin-4-yl, (1,1-dioxo-1,4-thiazinan-4-yl), [1-(2-hydroxypropanoyl)pyrrolidin-3-yl]oxy, [1-(2-hydroxypropanoyl)pyrrolidin-3-yl]oxy, (1-acetylpyrrolidin-3-yl)oxy, (1-methylsulfonylpyrrolidin-3-yl)oxy, pyrrolidin-3-yloxy, pyrrolidin-2-ylmethoxy, (1-acetylpyrrolidin-2-yl)methoxy, (1-methylpyrrolidin-3-yl)oxy, [1-(2-hydroxypropanoyl)piperidin-4-yl]oxy, (1-acetylpiperidin-4-yl)oxy, [1-(2-hydroxypropanoyl)piperidin-4-yl]oxy, (1-methylsulfonylpiperidin-4-yl)oxy, piperidin-4-yloxy, [1-(2-hydroxypropanoyl)azetidin-3-yl]oxy, [1-(2-methoxypropanoyl)azetidin-3-yl]oxy, (1-acetylazetidin-3-yl)oxy, [1-(1-hydroxycyclopropanecarbonyl)azetidin-3-yl]oxy, [1-(2-hydroxyacetyl)azetidin-3-yl]oxy, [1-(dimethylcarbamoyl)azetidin-3-yl]oxy, [1-(2-hydroxyethyl-methylcarbamoyl)azetidin-3-yl]oxy, [1-(2-hydroxypropyl)azetidin-3-yl]oxy, [1-(2-hydroxy-2-methylpropyl)azetidin-3-yl]oxy, [1-(2-hydroxyethyl)azetidin-3-yl]oxy, azetidin-3-yloxy, 2-[4-(2-methoxyethyl)piperazin-1-yl]ethoxy, formyl, carboxy, carboxymethyl, (1R)-1-(methylcarbamoyl)ethoxy, (1S)-1-(methylcarbamoyl)ethoxy, (1S,4S)-2-acetyl-2,5-diazabicyclo[2.2.1]hept-5-yl, (2-dimethylaminoacetyl)amino, (3R)-4-(2-hydroxyethyl)-3-methyl-piperazin-1-yl, (3S)-4-(2-hydroxyethyl)-3-methyl-piperazin-1-yl, 1-(2-hydroxyacetyl)-4-piperidyl, 1-(2-methylaminoacetyl)-4-piperidyl, 1-(acetamidomethyl)-2-hydroxy-ethyl, 1-(dimethylcarbamoylmethyl)-4-piperidyl, 1-[(2R)-2-aminopropanoyl]-4-piperidyl, 1-[(2S)-2-aminopropanoyl]-4-piperidyl, 1-acetylazetidin-3-yl, 2-(2-hydroxyethyl-methyl-amino)ethyl-methyl-amino, 2-[(2-hydroxyacetyl)-methyl-amino]ethyl-methyl-amino, 2-oxopiperazin-1-yl, 3-(2-aminoacetyl)-9-oxa-3,7-diazabicyclo[3.3.1]non-7-yl, 3-(2-dimethylaminoacetyl)-9-oxa-3,7-diazabicyclo[3.3.1]non-7-yl, 3-(2-hydroxyacetyl)-9-oxa-3,7-diazabicyclo[3.3.1]non-7-yl, 3-(2-hydroxyethyl)-9-oxa-3,7-diazabicyclo[3.3.1]non-7-yl, 3-(2-methylaminoacetyl)-9-oxa-3,7-diazabicyclo[3.3.1]non-7-yl, 3-(2-methylsulfonylethyl)-9-oxa-3,7-diazabicyclo[3.3.1]non-7-yl, 3-(3-hydroxypropyl)-9-oxa-3,7-diazabicyclo[3.3.1]non-7-yl, 3-(dimethylcarbamoylmethyl)-9-oxa-3,7-diazabicyclo[3.3.1]non-7-yl, 3-[(2R)-2-hydroxypropanoyl]-9-oxa-3,7-diazabicyclo[3.3.1]non-7-yl, 3-[(2S)-2-amino-3-hydroxy-propanoyl]-9-oxa-3,7-diazabicyclo[3.3.1]non-7-yl, 3-[(2S)-2-hydroxypropanoyl]-9-oxa-3,7-diazabicyclo[3.3.1]non-7-yl, 3-[2-(methylcarbamoyl)ethyl]-9-oxa-3,7-diazabicyclo[3.3.1]non-7-yl, 3-[2-hydroxy-1-(hydroxymethyl)ethyl]-9-oxa-3,7-diazabicyclo[3.3.1]non-7-yl, 3-acetyl-9-oxa-3,7-diazabicyclo[3.3.1]non-7-yl, 3-hydroxy-2-(hydroxymethyl)propyl, 3-methyl-9-oxa-3,7-diazabicyclo[3.3.1]non-7-yl, 3-oxa-7,9-diazabicyclo[3.3.1]non-7-yl, 4-(2-dimethylaminoacetyl)piperazin-1-yl, 4-(2-hydroxy-1,1-dimethyl-ethyl)piperazin-1-yl, 4-(2-hydroxy-1-methyl-ethyl)piperazin-1-yl, 4-(2-morpholinoacetyl)piperazin-1-yl, 4-(methylcarbamoyl)-1-piperidyl, 4-[(2S)-2,3-dihydroxypropyl]piperazin-1-yl, 4-[2-hydroxy-1-(hydroxymethyl)ethyl]piperazin-1-yl, 9-acetyl-3-oxa-7,9-diazabicyclo[3.3.1]non-7-yl, 9-oxa-3,7-diazabicyclo[3.3.1]non-7-yl, bis(2-hydroxyethyl)amino, cyano, dimethylcarbamoylmethoxy, dimethylcarbamoylmethyl-methyl-amino, methoxy, methyl, methyl-(2-oxo-2-piperazin-1-yl-ethyl)amino and methyl-(methylcarbamoylmethyl)amino.

A particular group of suitable values for R⁴ includes, for example, [1-[4-(2-hydroxyethyl)piperazin-1-yl]-1-oxopropan-2-yl], (1-oxo-1-piperazin-1-ylpropan-2-yl), (1-acetylpiperidin-2-yl), (1-acetylpiperidin-3-yl), (1-acetyl-2-methylpiperidin-4-yl), [1-(2-hydroxypropanoyl)piperidin-4-yl], (1-acetylpiperidin-4-yl), [1-(2-methoxypropanoyl)piperidin-4-yl], [1-(dimethylcarbamoyl)piperidin-4-yl], [1-(2-hydroxyethyl-methylcarbamoyl)piperidin-4-yl], (2-methylpiperidin-4-yl), piperidin-4-yl, [2-[4-(2-hydroxyethyl)piperazin-1-yl]-2-oxoethyl], (2-oxo-2-piperazin-1-ylethyl), (4-carbamoylpiperidin-1-yl)methyl, (4-hydroxypiperidin-1-yl)methyl, (3-hydroxypiperidin-1-yl)methyl, (4-methylpiperazin-1-yl)methyl, (4-acetylpiperazin-1-yl)methyl, [4-(2-hydroxyethyl)piperazin-1-yl]methyl, (4-acetyl-2,5-dimethylpiperazin-1-yl), (4-acetyl-3,5-dimethylpiperazin-1-yl), (3,5-dimethylpiperazin-1-yl), (4-hydroxypiperidin-1-yl), (4-methoxypiperidin-1-yl), (4-acetyl-3-methylpiperazin-1-yl), [4-acetyl-3-(hydroxymethyl)piperazin-1-yl], (4-methylpiperazin-1-yl), [4-(2-amino-3-methylbutanoyl)piperazin-1-yl], [4-(2-aminopropanoyl)piperazin-1-yl], [4-(2-hydroxypropanoyl)piperazin-1-yl], [4-(2,4-diamino-4-oxobutanoyl)piperazin-1-yl], [4-(2-amino-3-hydroxypropanoyl)piperazin-1-yl], [4-(2,3-dihydroxypropanoyl)piperazin-1-yl], [4-(4-hydroxypyrrolidine-2-carbonyl)piperazin-1-yl], [4-(5-oxopyrrolidine-2-carbonyl)piperazin-1-yl], [4-(piperidine-2-carbonyl)piperazin-1-yl], [4-(pyrrolidine-2-carbonyl)piperazin-1-yl], (4-acetylpiperazin-1-yl), [4-(2-methoxypropanoyl)piperazin-1-yl], [4-(2-amino-4-hydroxybutanoyl)piperazin-1-yl], [4-(2-hydroxy-2-methylpropanoyl)piperazin-1-yl], [4-(1-hydroxycyclopropanecarbonyl)piperazin-1-yl], [4-(5-oxopyrrolidine-2-carbonyl)piperazin-1-yl], (4-propanoylpiperazin-1-yl), [4-(2-aminoacetyl)piperazin-1-yl], [4-(2-methylaminoacetyl)piperazin-1-yl], [4-(2-acetamidoacetyl)piperazin-1-yl], [4-(2-ethylaminoacetyl)piperazin-1-yl], [4-(2-hydroxyacetyl)piperazin-1-yl], [4-(2-methoxyacetyl)piperazin-1-yl], (4-carbamoylpiperazin-1-yl), [4-(dimethylcarbamoyl)piperazin-1-yl], [4-(2-hydroxyethyl-methylcarbamoyl)piperazin-1-yl], [4-(piperazine-1-carbonyl)piperazin-1-yl], [4-(morpholine-4-carbonyl)piperazin-1-yl], (4-methoxycarbonylpiperazin-1-yl), [4-(2-methoxyethoxycarbonyl)piperazin-1-yl], [4-(2-hydroxypropyl)piperazin-1-yl], (4-ethylpiperazin-1-yl), [4-(2,3-dihydroxypropyl)piperazin-1-yl], [4-(2-dimethylamino-2-oxo ethyl)piperazin-1-yl], [4-[2-(2-hydroxyethyl-methylamino)-2-oxoethyl]piperazin-1-yl], [4-(2-morpholin-4-yl-2-oxoethyl)piperazin-1-yl], [4-(2-methylamino-2-oxoethyl)piperazin-1-yl], [4-(2-hydroxy-2-methylpropyl)piperazin-1-yl], [4-(2-cyano ethyl)piperazin-1-yl], [4-(2-hydroxyethyl)piperazin-1-yl], [4-(2-methoxyethyl)piperazin-1-yl], [4-(2-methylsulfonylethyl)piperazin-1-yl], (4-methylsulfonylpiperazin-1-yl), [4-(dimethylsulfamoyl)piperazin-1-yl], (3-oxopiperazin-1-yl), [3-(dimethylcarbamoyl)piperazin-1-yl], [4-(cyclopropanecarbonyl)piperazin-1-yl], [4-(pyrrolidine-2-carbonyl)piperazin-1-yl], piperazin-1-yl, [1-(2-hydroxypropanoyl)piperidin-4-yl]oxy, (1-acetylpiperidin-4-yl)oxy, [1-(2-hydroxypropanoyl)piperidin-4-yl]oxy, (1-methylsulfonylpiperidin-4-yl)oxy and piperidin-4-yloxy.

In an embodiment, there is provided a compound of formula (IA):

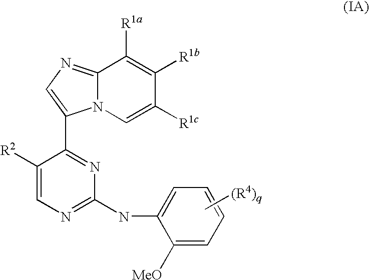

or a pharmaceutically acceptable salt thereof,
wherein:

each R^{1a}, R^{1b} and R^{1c}, which may be the same or different, is selected from hydrogen, halogeno, cyano, (C1-C6)alkyl, (C1-C6)alkoxy, amino, (C1-C6)alkylamino and di-[(C1-C6)alkyl]amino, each of which groups within R^{1a}, R^{1b} and R^{1c} may be optionally substituted by one or more substituents independently selected from hydroxy, halogeno, cyano, (C1-C6)alkyl, (C1-C6)alkoxy, amino, (C1-C6)alkylamino, di-[(C1-C6)alkyl]amino, —N(R″)C(O)R′ wherein R′ is selected from hydrogen, (C1-C6)alkyl and (C1-C6)alkoxy and R″ is selected from hydrogen and (C1-C6)alkyl, and a saturated monocyclic 4-, 5-, 6-, 7- or 8-membered ring optionally comprising one or more heteroatoms independently selected from nitrogen, oxygen and sulfur;

R² is selected from halogeno, cyano, trifluoromethyl, cyclopropyl, (C1-C3)alkyl and (C1-C3)alkoxy;

q represents 1, 2, 3 or 4;

each R⁴, which may be the same or different, is selected from hydroxy, cyano, halogeno, formyl, carboxy, (C1-C6)alkyl, (C2-C6)alkenyl, (C2-C6)alkynyl, (C1-C6)alkoxy, (C2-C6)alkanoyl, (C1-C6)alkoxycarbonyl, (C3-C8)cycloalkyl, (C3-C8)cycloalkylcarbonyl, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (C1-C6)alkoxyamino, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, sulfamoyl, (C1-C6)alkylsulfamoyl, di-[(C1-C6)alkyl]sulfamoyl, —S(O)ₘR′ wherein R′ is as defined above and m represents 0, 1 or 2, —N(R″)C(O)R′ wherein R′ and R″ are each as defined above, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

or two R⁴ groups on adjacent carbon atoms of the phenyl ring, together with the carbon atoms to which they are attached, form a saturated or unsaturated monocyclic 5- or 6-membered heterocyclic ring comprising at least one ring heteroatom selected from nitrogen, oxygen and sulfur,

each of which groups or rings within R⁴ may be optionally substituted by one or more substituents independently selected from hydroxy, halogeno, cyano, formyl, carboxy, (C1-C6)alkyl, (C3-C8)cycloalkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, (C1-C6)alkylthio, sulfamoyl, (C1-C6)alkylsulfamoyl, di-[(C1-C6)alkyl]sulfamoyl, —S(O)ₘR′ wherein R′ and m are each as defined above, —N(R″)C(O)R′ wherein R′ and R″ are each as defined above, and —X-Q wherein X and Q are each as defined above, any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ and m are each as defined above, —N(R″)C(O)R′ wherein R′ and R″ are each as defined above, and —X-Q wherein X and Q are each as defined above;

and wherein any saturated monocyclic ring optionally bears 1 or 2 oxo or thioxo substituents.

As the skilled person would appreciate, “Me” represents methyl.

In one aspect, in the compounds of formula (IA), each R^{1a}, R^{1b} and R^{1c}, which may be the same or different, may be selected from hydrogen, halogeno, cyano, methyl, ethyl, methoxy and dimethylamino, each of which groups within R^{1a}, R^{1b} and R^{1c} may be optionally substituted by one or more substituents independently selected from hydroxy and halogeno (especially hydroxy).

In another aspect, in the compounds of formula (IA), the groups R^{1a}, R^{1b} and R^{1c} all represent hydrogen.

In particular, in the compounds of formula (IA), R² may be halogeno, for example chloro, fluoro or bromo, especially chloro or fluoro. In one particular aspect, R² is chloro. In another particular aspect, R² is fluoro.

In particular, in the compounds of formula (IA), q may be 1 or 2, especially 1.

In particular, in the compounds of formula (IA), each R⁴, which may be the same or different, may be selected from (C1-C6)alkyl, (C1-C6)alkoxy and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

each of which groups or rings within R⁴ may be optionally substituted by one or more substituents independently selected from hydroxy, formyl, carboxy, (C1-C6)alkyl, (C3-C8)cycloalkylcarbonyl, (C1-C6)alkoxy, amino, di-[(C1-C6)alkyl]amino, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, di-[(C1-C6)alkyl]carbamoyl, (C1-C6)alkylcarbamoyl(C1-C6)alkyl, di-[(C1-C6)alkyl]carbamoyl(C1-C6)alkyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above (especially X is —C(O)—), any of which substituents may be optionally substituted by one or more further substituents independently selected from carboxy, (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above;

and wherein any saturated monocyclic ring optionally bears 1 or 2 oxo substituents.

In one aspect, in the compounds of formula (IA), each R⁴, which may be the same or different, may be selected from (C1-C6)alkyl, (C1-C6)alkoxy and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

each of which groups or rings within R⁴ may be optionally substituted by one or more substituents independently selected from hydroxy, formyl, (C1-C6)alkyl, (C3-C8)cycloalkylcarbonyl, (C1-C6)alkoxy, amino, di-[(C1-C6)alkyl]amino, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, di-[(C1-C6)alkyl]carbamoyl, (C1-C6)alkylcarbamoyl(C1-C6)alkyl, di-[(C1-C6)alkyl]carbamoyl(C1-C6)alkyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above (especially X is —C(O)—), any of which substituents may be optionally substituted by one or more further substituents independently selected from carboxy, (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above;

and wherein any saturated monocyclic ring optionally bears 1 or 2 oxo substituents.

In one aspect, in the compounds of formula (IA), each R⁴, which may be the same or different, may be selected from —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

each of which groups or rings within R⁴ may be optionally substituted by one or more substituents independently selected from hydroxy, (C1-C6)alkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above (especially X is —C(O)—), any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above;

and wherein any saturated monocyclic ring optionally bears 1 or 2 oxo substituents.

In another aspect, in the compounds of formula (IA), each R⁴, which may be the same or different, may be selected from —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

each of which groups or rings within R⁴ may be optionally substituted by one or more substituents independently selected from (C1-C6)alkyl and (2-6C)alkanoyl, any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ is selected from hydrogen and (C1-C6)alkyl and m represents 2, —N(R″)C(O)R′ wherein R′ is selected from hydrogen and (C1-C6)alkyl and R″ is selected from hydrogen and (C1-C6)alkyl, and —X-Q wherein X and Q are each as defined above;

and wherein any saturated monocyclic ring optionally bears 1 or 2 oxo substituents.

In another aspect, in the compounds of formula (IA), each R⁴, which may be the same or different, may be selected from —X-Q wherein X is a direct bond and Q represents a saturated 4-, 5-, 6-, 7-, 8-, 9- or 10-membered heterocyclic ring comprising at least one ring heteroatom selected from nitrogen, oxygen and sulfur,

each of which rings within R⁴ may be optionally substituted by one or more substituents independently selected from (C1-C6)alkyl and (2-6C)alkanoyl, any of which substituents may be optionally substituted by one or more further substituents independently selected from (C1-C4)alkyl, (C1-C4)alkoxy, hydroxy and hydroxy(C1-C4)alkyl, and wherein any saturated monocyclic ring optionally bears 1 or 2 oxo substituents.

In another aspect, in the compounds of formula (IA), each R⁴, which may be the same or different, may be selected from —X-Q wherein X is a direct bond and Q represents a saturated 4-, 5-, 6-, 7-, 8-, 9- or 10-membered heterocyclic ring comprising at least one ring nitrogen atom and optionally comprising a further one or two ring heteroatoms selected from nitrogen, oxygen and sulfur,

each of which rings within R⁴ may be optionally substituted by one or more substituents independently selected from (C1-C6)alkyl and (2-6C)alkanoyl, any of which substituents may be optionally substituted by one or more further substituents independently selected from (C1-C4)alkyl, (C1-C4)alkoxy, (C1-C4)alkylamino, di-[(C1-C4)alkyl]amino, hydroxy and hydroxy(C1-C4)alkyl,

and wherein any saturated monocyclic ring optionally bears 1 or 2 oxo substituents.

In a further embodiment, there is provided a compound of formula (IAi):

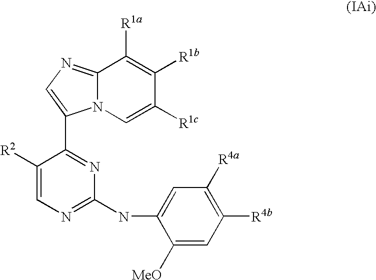

or a pharmaceutically acceptable salt thereof,
wherein:

each R^{1a}, R^{1b} and R^{1c}, which may be the same or different, is selected from hydrogen, halogen, cyano, (C1-C6)alkyl, (C1-C6)alkoxy, amino, (C1-C6)alkylamino and di-[(C1-C6)alkyl]amino, each of which groups within R^{1a}, R^{1b} and R^{1c} may be optionally substituted by one or more substituents independently selected from hydroxy, halogeno, cyano, (C1-C6)alkyl, (C1-C6)alkoxy, amino, (C1-C6)alkylamino, di-[(C1-C6)alkyl]amino, —N(R″)C(O)R′ wherein R′ is selected from hydrogen, (C1-C6)alkyl and (C1-C6)alkoxy and R″ is selected from hydrogen and (C1-C6)alkyl, and a saturated monocyclic 4-, 5-, 6-, 7- or 8-membered ring optionally comprising one or more heteroatoms independently selected from nitrogen, oxygen and sulfur;

R² is selected from halogeno, cyano, trifluoromethyl, cyclopropyl, (C1-C3)alkyl and (C1-C3)alkoxy; and

either one of R^{4a} and R^{4b} may be hydrogen and the remaining R^{4a} or R^{4b} is selected from hydroxy, cyano, halogeno, formyl, carboxy, (C1-C6)alkyl, (C2-C6)alkenyl, (C2-C6)alkynyl, (C1-C6)alkoxy, (C2-C6)alkanoyl, (C1-C6)alkoxycarbonyl, (C3-C8)cyclo alkyl, (C3-C8)cycloalkylcarbonyl, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (C1-C6)alkoxyamino, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, sulfamoyl, (C1-C6)alkylsulfamoyl, di-[(C1-C6)alkyl]sulfamoyl, —S(O)ₘR′ wherein R′ is as defined above and m represents 0, 1 or 2, —N(R″)C(O)R′ wherein R′ and R″ are each as defined above, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

or R^{4a} & R^{4b} groups on adjacent carbon atoms of the phenyl ring, together with the carbon atoms to which they are attached, form a saturated or unsaturated monocyclic 5- or 6-membered heterocyclic ring comprising at least one ring heteroatom selected from nitrogen, oxygen and sulfur,

each of which groups or rings within R^{4a} and R^{4b} may be optionally substituted by one or more substituents independently selected from hydroxy, halogeno, cyano, formyl, carboxy, (C1-C6)alkyl, (C3-C8)cyclo alkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, (C1-C6)alkylthio, sulfamoyl, (C1-C6)alkylsulfamoyl, di-[(C1-C6)alkyl]sulfamoyl, —S(O)ₘR′ wherein R′ and m are each as defined above, —N(R″)C(O)R′ wherein R′ and R″ are each as defined above, and —X-Q wherein X and Q are each as defined above, any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ and m are each as defined above, —N(R″)C(O)R′ wherein R′ and R″ are each as defined above, and —X-Q wherein X and Q are each as defined above; and wherein any saturated monocyclic ring optionally bears 1 or 2 oxo or thioxo substituents.

In one aspect, in the compounds of formula (IAi), each R^{1a}, R^{1b} and R^{1c}, which may be the same or different, may be selected from hydrogen, halogeno, cyano, methyl, ethyl, methoxy and dimethylamino, each of which groups within R^{1a}, R^{1b} and R^{1c} may be optionally substituted by one or more substituents independently selected from hydroxy and halogeno (especially hydroxy).

In another aspect, in the compounds of formula (IAi), the groups R^{1a}, R^{1b} and R^{1c} all represent hydrogen.

In particular, in the compounds of formula (IAi), R² may be halogeno, for example chloro, fluoro or bromo, especially chloro or fluoro. In one particular aspect, R² is chloro. In another particular aspect, R² is fluoro.

In one aspect, in the compounds of formula (IAi), R^{4a} & R^{4b} groups on adjacent carbon atoms of the phenyl ring, together with the carbon atoms to which they are attached, form a saturated or unsaturated monocyclic 5- or 6-membered heterocyclic ring comprising at least one ring heteroatom selected from nitrogen, oxygen and sulfur, and wherein the heterocyclic ring may be optionally substituted by one or more substituents independently selected from hydroxy, halogeno, cyano, formyl, carboxy, (C1-C6)alkyl, (C3-C8)cycloalkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, (C1-C6)alkylthio, sulfamoyl, (C1-C6)alkylsulfamoyl, di-[(C1-C6)alkyl]sulfamoyl, —S(O)ₘR′ wherein R′ and m are each as defined above, —N(R″)C(O)R′ wherein R′ and R″ are each as defined above, and —X-Q wherein X and Q are each as defined above, any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ and m are each as defined above, —N(R″)C(O)R′ wherein R′ and R″ are each as defined above, and —X-Q wherein X and Q are each as defined above;

and wherein any saturated monocyclic ring optionally bears 1 or 2 oxo or thioxo substituents.

In particular, in the compounds of formula (IAi), R^{4a} & R^{4b} groups on adjacent carbon atoms of the phenyl ring, together with the carbon atoms to which they are attached, form a saturated or unsaturated monocyclic 5- or 6-membered heterocyclic ring comprising one ring nitrogen and wherein the heterocyclic ring may be optionally substituted by one or more substituents independently selected from hydroxy, halogeno, cyano, formyl, carboxy, (C1-C3)alkyl, (C3)cycloalkyl, (C3)cycloalkylcarbonyl, (C1-C3)alkoxy, amino, (C1-C3)alkylamino, di-[(C1-C3)alkyl]amino, amino(C1-C3)alkyl, (C1-C3)alkylamino(C1-C3)alkyl, di-[(C1-C3)alkyl]amino(C1-C3)alkyl, (2-3C)alkanoyl, (C1-C3)alkoxycarbonyl, carbamoyl, (C1-C3)alkylcarbamoyl, di-[(C1-C3)alkyl]carbamoyl, carbamoyl(C1-C3)alkyl, (C1-C3)alkylcarbamoyl(C1-C3)alkyl, di-[(C1-C3)alkyl]carbamoyl(C1-C3)alkyl, (C1-C3)alkylthio, sulfamoyl, (C1-C3)alkylsulfamoyl, di-[(C1-C3)alkyl]sulfamoyl, —S(O)ₘR′ wherein R′ and m are each as defined above, —N(R″)C(O)R′ wherein R′ and R″ are each as defined above, and —X-Q wherein X and Q are each as defined above, any of which substituents may be optionally substituted by one or more further substituents independently selected from (C1-C3)alkyl, (C1-C3)alkoxy, hydroxy, halogeno, cyano, hydroxy(C1-C3)alkyl, amino, (C1-C3)alkylamino, di-[(C1-C3)alkyl]amino, carbamoyl, (C1-C3)alkylcarbamoyl, di-[(C1-C3)alkyl]carbamoyl, —S(O)ₘR′ wherein R′ and m are each as defined above, —N(R″)C(O)R′ wherein R′ and R″ are each as defined above, and —X-Q wherein X and Q are each as defined above;

and wherein any saturated monocyclic ring optionally bears 1 or 2 oxo or thioxo substituents.

In particular, in the compounds of formula (IAi), R^{4a} & R^{4b} groups on adjacent carbon atoms of the phenyl ring, together with the carbon atoms to which they are attached, form a saturated or unsaturated monocyclic 5-membered heterocyclic ring comprising one ring nitrogen which is attached to one of the carbon atoms of the phenyl ring and wherein the heterocyclic ring may be optionally substituted on carbon by one or more substituents independently selected from (C1-C3)alkyl and is substituted on the ring nitrogen by (2-3C)alkanoyl which may be optionally substituted by one or more further substituents independently selected from (C1-C3)alkyl, (C1-C3)alkoxy, hydroxy, halogeno, hydroxy(C1-C3)alkyl, amino, (C1-C3)alkylamino and di-[(C1-C3)alkyl]amino.

In particular, in the compounds of formula (IAi), R^{4a} & R^{4b} groups on adjacent carbon atoms of the phenyl ring, together with the carbon atoms to which they are attached, form a saturated or unsaturated monocyclic 5-membered heterocyclic ring comprising one ring nitrogen which is fused to the phenyl ring in such a way as to form an indolinyl ring and wherein the heterocyclic ring of the indolinyl ring may be optionally substituted on carbon by one or more substituents independently selected from (C1-C3)alkyl and is substituted on the ring nitrogen by (2-3C)alkanoyl which may be optionally substituted by one or more further substituents independently selected from (C1-C3)alkyl, (C1-C3)alkoxy, hydroxy, halogeno, cyano, hydroxy(C1-C3)alkyl, amino, (C1-C3)alkylamino, di-[(C1-C3)alkyl]amino, carbamoyl, (C1-C3)alkylcarbamoyl, di-[(C1-C3)alkyl]carbamoyl, —S(O)ₘR′ wherein R′ and m are each as defined above, —N(R″)C(O)R′ wherein R′ and R″ are each as defined above, and —X-Q wherein X and Q are each as defined above.

In particular, in the compounds of formula (IAi), R^{4a} & R^{4b} groups on adjacent carbon atoms of the phenyl ring, together with the carbon atoms to which they are attached, form a saturated or unsaturated monocyclic 5-membered heterocyclic ring comprising one ring nitrogen which is fused to the phenyl ring in such a way as to form an indolinyl ring and wherein the heterocyclic ring of the indolinyl ring is substituted on the ring nitrogen by (2-3C)alkanoyl which may be optionally substituted by one or more further substituents independently selected from (C1-C3)alkyl, (C1-C3)alkoxy, hydroxy, halogeno, hydroxy(C1-C3)alkyl, amino, (C1-C3)alkylamino and di-[(C1-C3)alkyl]amino.

In one aspect, in the compounds of formula (IAi), one of R^{4a} and R^{4b} is hydrogen and the remaining R^{4a} or R^{4b} is selected from hydroxy, cyano, halogeno, formyl, carboxy, (C1-C6)alkyl, (C2-C6)alkenyl, (C2-C6)alkynyl, (C1-C6)alkoxy, (C2-C6)alkanoyl, (C1-C6)alkoxycarbonyl, (C3-C8)cycloalkyl, (C3-C8)cycloalkylcarbonyl, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (C1-C6)alkoxyamino, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, sulfamoyl, (C1-C6)alkylsulfamoyl, di-[(C1-C6)alkyl]sulfamoyl, —S(O)ₘR′ wherein R′ is as defined above and m represents 0, 1 or 2, —N(R″)C(O)R′ wherein R′ and R″ are each as defined above, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

wherein each of the groups or rings within R^{4a} and R^{4b} may be optionally substituted by one or more substituents independently selected from hydroxy, halogeno, cyano, formyl, carboxy, (C1-C6)alkyl, (C3-C8)cycloalkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, (C1-C6)alkylthio, sulfamoyl, (C1-C6)alkylsulfamoyl, di-[(C1-C6)alkyl]sulfamoyl, —S(O)ₘR′ wherein R′ and m are each as defined above, —N(R″)C(O)R′ wherein R′ and R″ are each as defined above, and —X-Q wherein X and Q are each as defined above, any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ and m are each as defined above, —N(R″)C(O)R′ wherein R′ and R″ are each as defined above, and —X-Q wherein X and Q are each as defined above;

and wherein any saturated monocyclic ring optionally bears 1 or 2 oxo or thioxo substituents.

In another aspect, in the compounds of formula (IAi), one of R^{4a} and R^{4b} is hydrogen and the remaining R^{4a} or R^{4b} is —X-Q wherein X is a direct bond and Q represents a saturated 4-, 5-, 6-, 7-, 8-, 9- or 10-membered heterocyclic ring comprising at least one ring nitrogen atom and optionally comprising a further one or two ring heteroatoms selected from nitrogen, oxygen and sulfur,

each of which rings within R^{4a} or R^{4b} may be optionally substituted by one or more substituents independently selected from (C1-C6)alkyl and (2-6C)alkanoyl, any of which substituents may be optionally substituted by one or more further substituents independently selected from (C1-C4)alkyl, (C1-C4)alkoxy, (C1-C4)alkylamino, di-[(C1-C4)alkyl]amino, hydroxy and hydroxy(C1-C4)alkyl,

and wherein any saturated monocyclic ring optionally bears 1 or 2 oxo substituents.

In another aspect, in the compounds of formula (IAi), one of R^{4a} and R^{4b} is hydrogen and the remaining R^{4a} or R^{4b} is —X-Q wherein X is a direct bond and Q represents a piperazin-1-yl which may be optionally substituted by one or more substituents independently selected from (C1-C6)alkyl and (2-6C)alkanoyl, any of which substituents may be optionally substituted by one or more further substituents independently selected from (C1-C4)alkyl, (C1-C4)alkoxy, (C1-C4)alkylamino, di-[(C1-C4)alkyl]amino, hydroxy and hydroxy(C1-C4)alkyl,

and wherein any saturated monocyclic ring optionally bears 1 or 2 oxo substituents.

In an embodiment, there is provided a compound of formula (IB):

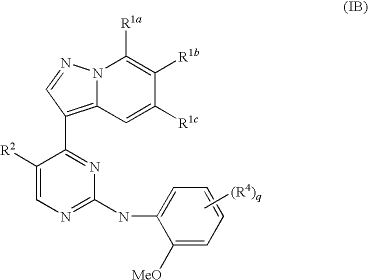

or a pharmaceutically acceptable salt thereof,
wherein:

each R^{1a}, R^{1b} and R^{1c}, which may be the same or different, is selected from hydrogen, halogeno, cyano, (C1-C6)alkyl, (C1-C6)alkoxy, amino, (C1-C6)alkylamino and di-[(C1-C6)alkyl]amino, each of which groups within R^{1a}, R^{1b} and R^{1c} may be optionally substituted by one or more substituents independently selected from hydroxy, halogeno, cyano, (C1-C6)alkyl, (C1-C6)alkoxy, amino, (C1-C6)alkylamino, di-[(C1-C6)alkyl]amino, —N(R″)C(O)R′ wherein R′ is selected from hydrogen, (C1-C6)alkyl and (C1-C6)alkoxy and R″ is selected from hydrogen and (C1-C6)alkyl, and a saturated monocyclic 4-, 5-, 6-, 7- or 8-membered ring optionally comprising one or more heteroatoms independently selected from nitrogen, oxygen and sulfur;

R² is selected from halogeno, cyano, trifluoromethyl, cyclopropyl, (C1-C3)alkyl and (C1-C3)alkoxy;

q represents 1, 2, 3 or 4;

each R⁴, which may be the same or different, is selected from hydroxy, cyano, halogeno, (C1-C6)alkyl, (C2-C6)alkenyl, (C2-C6)alkynyl, (C1-C6)alkoxy, (C2-C6)alkanoyl, (C1-C6)alkoxycarbonyl, (C3-C8)cycloalkyl, (C3-C8)cycloalkylcarbonyl, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (C1-C6)alkoxyamino, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, sulfamoyl, (C1-C6)alkylsulfamoyl, di-[(C1-C6)alkyl]sulfamoyl, —S(O)ₘR′ wherein R′ is as defined above and m represents 0, 1 or 2, —N(R″)C(O)R′ wherein R′ and R″ are each as defined above, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

or two R⁴ groups on adjacent carbon atoms of the phenyl ring, together with the carbon atoms to which they are attached, form a saturated or unsaturated monocyclic 5- or 6-membered heterocyclic ring comprising at least one ring heteroatom selected from nitrogen, oxygen and sulfur,

each of which groups or rings within R⁴ may be optionally substituted by one or more substituents independently selected from hydroxy, halogeno, cyano, (C1-C6)alkyl, (C3-C8)cycloalkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, (C1-C6)alkylthio, sulfamoyl, (C1-C6)alkylsulfamoyl, di-[(C1-C6)alkyl]sulfamoyl, —S(O)ₘR′ wherein R′ and m are each as defined above, —N(R″)C(O)R′ wherein R′ and R″ are each as defined above, and —X-Q wherein X and Q are each as defined above, any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ and m are each as defined above, —N(R″)C(O)R′ wherein R′ and R″ are each as defined above, and —X-Q wherein X and Q are each as defined above;

and wherein any saturated monocyclic ring optionally bears 1 or 2 oxo or thioxo substituents.

In one aspect, in the compounds of formula (IB), each R^{1a}, R^{1b} and R^{1c}, which may be the same or different, may be selected from hydrogen, halogeno, cyano, methyl, ethyl, methoxy and dimethylamino, each of which groups within R^{1a}, R^{1b} and R^{1c} may be optionally substituted by one or more substituents independently selected from hydroxy and halogeno (especially hydroxy).

In another aspect, in the compounds of formula (IB), the groups R^{1a}, R^{1b} and R^{1c} all represent hydrogen.

In particular, in the compounds of formula (IB), R² may be halogeno, for example chloro, fluoro or bromo, especially chloro or fluoro. In one particular aspect, R² is chloro. In another particular aspect, R² is fluoro.

In a further particular aspect, R² is methyl.

In particular, in the compounds of formula (IB), q may be 1 or 2, especially 1.

In particular, in the compounds of formula (IB), each R⁴, which may be the same or different, may be selected from (C1-C6)alkyl, (C1-C6)alkoxy and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

each of which groups or rings within R⁴ may be optionally substituted by one or more substituents independently selected from hydroxy, (C1-C6)alkyl, (C3-C8)cycloalkylcarbonyl, (C1-C6)alkoxy, amino, di-[(C1-C6)alkyl]amino, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, di-[(C1-C6)alkyl]carbamoyl, (C1-C6)alkylcarbamoyl(C1-C6)alkyl, di-[(C1-C6)alkyl]carbamoyl(C1-C6)alkyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above (especially X is —C(O)—), any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above;

and wherein any saturated monocyclic ring optionally bears 1 or 2 oxo substituents.

In one aspect, in the compounds of formula (IB), each R⁴, which may be the same or different, may be selected from —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

each of which groups or rings within R⁴ may be optionally substituted by one or more substituents independently selected from hydroxy, (C1-C6)alkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above (especially X is —C(O)—), any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ is selected from hydrogen and (C1-C6)alkyl and m represents 0, 1 or 2 (especially m represents 2), —N(R″)C(O)R′ wherein R′ is selected from hydrogen and (C1-C6)alkyl and R″ is selected from hydrogen and (C1-C6)alkyl, and —X-Q wherein X and Q are each as defined above;

and wherein any saturated monocyclic ring optionally bears 1 or 2 oxo substituents.

In another aspect, in the compounds of formula (IB), each R⁴, which may be the same or different, may be selected from —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

each of which groups or rings within R⁴ may be optionally substituted by one or more substituents independently selected from (C1-C6)alkyl and (2-6C)alkanoyl, any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ is selected from hydrogen and (C1-C6)alkyl and m represents 2, —N(R″)C(O)R′ wherein R′ is selected from hydrogen and (C1-C6)alkyl and R″ is selected from hydrogen and (C1-C6)alkyl, and —X-Q wherein X and Q are each as defined above;

and wherein any saturated monocyclic ring optionally bears 1 or 2 oxo substituents.

In another aspect, in the compounds of formula (IB), each R⁴, which may be the same or different, may be selected from —X-Q wherein X is a direct bond and Q represents a saturated 4-, 5-, 6-, 7-, 8-, 9- or 10-membered heterocyclic ring comprising at least one ring heteroatom selected from nitrogen, oxygen and sulfur,

each of which rings within R⁴ may be optionally substituted by one or more substituents independently selected from (C1-C6)alkyl and (2-6C)alkanoyl, any of which substituents may be optionally substituted by one or more further substituents independently selected from (C1-C4)alkyl, (C1-C4)alkoxy, hydroxy and hydroxy(C1-C4)alkyl, and wherein any saturated monocyclic ring optionally bears 1 or 2 oxo substituents.

In a further embodiment, there is provided a compound of formula (IBi):

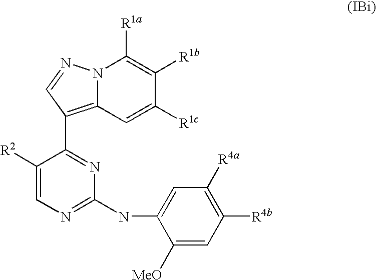

or a pharmaceutically acceptable salt thereof,
wherein:

each R^{1a}, R^{1b} and R^{1c}, which may be the same or different, is selected from hydrogen, halogeno, cyano, (C1-C6)alkyl, (C1-C6)alkoxy, amino, (C1-C6)alkylamino and di-[(C1-C6)alkyl]amino, each of which groups within R^{1a}, R^{1b} and R^{1c} may be optionally substituted by one or more substituents independently selected from hydroxy, halogeno, cyano, (C1-C6)alkyl, (C1-C6)alkoxy, amino, (C1-C6)alkylamino, di-[(C1-C6)alkyl]amino, —N(R″)C(O)R′ wherein R′ is selected from hydrogen, (C1-C6)alkyl and (C1-C6)alkoxy and R″ is selected from hydrogen and (C1-C6)alkyl, and a saturated monocyclic 4-, 5-, 6-, 7- or 8-membered ring optionally comprising one or more heteroatoms independently selected from nitrogen, oxygen and sulfur;

R² is selected from halogeno, cyano, trifluoromethyl, cyclopropyl, (C1-C3)alkyl and (C1-C3)alkoxy; and

either one of R^{4a} or R^{4b} is hydrogen and the remaining R^{4a} or R^{4b} is selected from hydroxy, cyano, halogeno, formyl, carboxy, (C1-C6)alkyl, (C2-C6)alkenyl, (C2-C6)alkynyl, (C1-C6)alkoxy, (C2-C6)alkanoyl, (C1-C6)alkoxycarbonyl, (C3-C8)cycloalkyl, (C3-C8)cycloalkylcarbonyl, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (C1-C6)alkoxyamino, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, sulfamoyl, (C1-C6)alkylsulfamoyl, di-[(C1-C6)alkyl]sulfamoyl, —S(O)ₘR′ wherein R′ is as defined above and m represents 0, 1 or 2, —N(R″)C(O)R′ wherein R′ and R″ are each as defined above, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

or R^{4a} & R^{4b} groups on adjacent carbon atoms of the phenyl ring, together with the carbon atoms to which they are attached, form a saturated or unsaturated monocyclic 5- or 6-membered heterocyclic ring comprising at least one ring heteroatom selected from nitrogen, oxygen and sulfur,

each of which groups or rings within R^{4a} and R^{4b} may be optionally substituted by one or more substituents independently selected from hydroxy, halogeno, cyano, formyl, carboxy, (C1-C6)alkyl, (C3-C8)cycloalkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, (C1-C6)alkylthio, sulfamoyl, (C1-C6)alkylsulfamoyl, di-[(C1-C6)alkyl]sulfamoyl, —S(O)ₘR′ wherein R′ and m are each as defined above, —N(R″)C(O)R′ wherein R′ and R″ are each as defined above, and —X-Q wherein X and Q are each as defined above, any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ and m are each as defined above, —N(R″)C(O)R′ wherein R′ and R″ are each as defined above, and —X-Q wherein X and Q are each as defined above;

and wherein any saturated monocyclic ring optionally bears 1 or 2 oxo or thioxo substituents.

In one aspect, in the compounds of formula (IBi), each R^{1a}, R^{1b} and R^{1c}, which may be the same or different, may be selected from hydrogen, halogeno, cyano, methyl, ethyl, methoxy and dimethylamino, each of which groups within R^{1a}, R^{1b} and R^{1c} may be optionally substituted by one or more substituents independently selected from hydroxy and halogeno (especially hydroxy).

In another aspect, in the compounds of formula (IBi), the groups R^{1a}, R^{1b} and R^{1c} all represent hydrogen.

In particular, in the compounds of formula (IBi), R² may be halogeno, for example chloro, fluoro or bromo, especially chloro or fluoro. In one particular aspect, R² is chloro. In another particular aspect, R² is fluoro.

In a further particular aspect, R² is methyl.

In one aspect, in the compounds of formula (IBi), R^{4a} & R^{4b} groups on adjacent carbon atoms of the phenyl ring, together with the carbon atoms to which they are attached, form a saturated or unsaturated monocyclic 5- or 6-membered heterocyclic ring comprising at least one ring heteroatom selected from nitrogen, oxygen and sulfur, and wherein the heterocyclic ring may be optionally substituted by one or more substituents independently selected from hydroxy, halogeno, cyano, formyl, carboxy, (C1-C6)alkyl, (C3-C8)cycloalkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, (C1-C6)alkylthio, sulfamoyl, (C1-C6)alkylsulfamoyl, di-[(C1-C6)alkyl]sulfamoyl, —S(O)ₘR′ wherein R′ and m are each as defined above, —N(R″)C(O)R′ wherein R′ and R″ are each as defined above, and —X-Q wherein X and Q are each as defined above, any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ and m are each as defined above, —N(R″)C(O)R′ wherein R′ and R″ are each as defined above, and —X-Q wherein X and Q are each as defined above;

and wherein any saturated monocyclic ring optionally bears 1 or 2 oxo or thioxo substituents.

In particular, in the compounds of formula (IBi), R^{4a} & R^{4b} groups on adjacent carbon atoms of the phenyl ring, together with the carbon atoms to which they are attached, form a saturated or unsaturated monocyclic 5- or 6-membered heterocyclic ring comprising one ring nitrogen and wherein the heterocyclic ring may be optionally substituted by one or more substituents independently selected from hydroxy, halogeno, cyano, formyl, carboxy, (C1-C3)alkyl, (C3)cycloalkyl, (C3)cycloalkylcarbonyl, (C1-C3)alkoxy, amino, (C1-C3)alkylamino, di-[(C1-C3)alkyl]amino, amino(C1-C3)alkyl, (C1-C3)alkylamino(C1-C3)alkyl, di-[(C1-C3)alkyl]amino(C1-C3)alkyl, (2-3C)alkanoyl, (C1-C3)alkoxycarbonyl, carbamoyl, (C1-C3)alkylcarbamoyl, di-[(C1-C3)alkyl]carbamoyl, carbamoyl(C1-C3)alkyl, (C1-C3)alkylcarbamoyl(C1-C3)alkyl, di-[(C1-C3)alkyl]carbamoyl(C1-C3)alkyl, (C1-C3)alkylthio, sulfamoyl, (C1-C3)alkylsulfamoyl, di-[(C1-C3)alkyl]sulfamoyl, —S(O)ₘR′ wherein R′ and m are each as defined above, —N(R″)C(O)R′ wherein R′ and R″ are each as defined above, and —X-Q wherein X and Q are each as defined above, any of which substituents may be optionally substituted by one or more further substituents independently selected from (C1-C3)alkyl, (C1-C3)alkoxy, hydroxy, halogeno, cyano, hydroxy(C1-C3)alkyl, amino, (C1-C3)alkylamino, di-[(C1-C3)alkyl]amino, carbamoyl, (C1-C3)alkylcarbamoyl, di-[(C1-C3)alkyl]carbamoyl, —S(O)ₘR′ wherein R′ and m are each as defined above, —N(R″)C(O)R′ wherein R′ and R″ are each as defined above, and —X-Q wherein X and Q are each as defined above;

and wherein any saturated monocyclic ring optionally bears 1 or 2 oxo or thioxo substituents.

In particular, in the compounds of formula (IBi), R^{4a} & R^{4b} groups on adjacent carbon atoms of the phenyl ring, together with the carbon atoms to which they are attached, form a saturated or unsaturated monocyclic 5-membered heterocyclic ring comprising one ring nitrogen which is attached to one of the carbon atoms of the phenyl ring and wherein the heterocyclic ring may be optionally substituted on carbon by one or more substituents independently selected from (C1-C3)alkyl and is substituted on the ring nitrogen by (2-3C)alkanoyl which may be optionally substituted by one or more further substituents independently selected from (C1-C3)alkyl, (C1-C3)alkoxy, hydroxy, halogeno, cyano, hydroxy(C1-C3)alkyl, amino, (C1-C3)alkylamino, di-[(C1-C3)alkyl]amino, carbamoyl, (C1-C3)alkylcarbamoyl, di-[(C1-C3)alkyl]carbamoyl, —S(O)ₘR′ wherein R′ and m are each as defined above, —N(R″)C(O)R′ wherein R′ and R″ are each as defined above, and —X-Q wherein X and Q are each as defined above.

In particular, in the compounds of formula (IBi), R^{4a} & R^{4b} groups on adjacent carbon atoms of the phenyl ring, together with the carbon atoms to which they are attached, form a saturated or unsaturated monocyclic 5-membered heterocyclic ring comprising one ring nitrogen which is fused to the phenyl ring in such a way as to form an indolinyl ring and wherein the heterocyclic ring of the indolinyl ring may be optionally substituted on carbon by one or more substituents independently selected from (C1-C3)alkyl and is substituted on the ring nitrogen by (2-3C)alkanoyl which may be optionally substituted by one or more further substituents independently selected from (C1-C3)alkyl, (C1-C3)alkoxy, hydroxy, halogeno, cyano, hydroxy(C1-C3)alkyl, amino, (C1-C3)alkylamino, di-[(C1-C3)alkyl]amino, carbamoyl, (C1-C3)alkylcarbamoyl, di-[(C1-C3)alkyl]carbamoyl, —S(O)ₘR′ wherein R′ and m are each as defined above, —N(R″)C(O)R′ wherein R′ and R″ are each as defined above, and —X-Q wherein X and Q are each as defined above.

In particular, in the compounds of formula (IBi), R^{4a} & R^{4b} groups on adjacent carbon atoms of the phenyl ring, together with the carbon atoms to which they are attached, form a saturated or unsaturated monocyclic 5-membered heterocyclic ring comprising one ring nitrogen which is fused to the phenyl ring in such a way as to form an indolinyl ring and wherein the heterocyclic ring of the indolinyl ring is substituted on the ring nitrogen by (2-3C)alkanoyl which may be optionally substituted by one or more further substituents independently selected from (C1-C3)alkyl, (C1-C3)alkoxy, hydroxy, halogeno, hydroxy(C1-C3)alkyl, amino, (C1-C3)alkylamino and di-[(C1-C3)alkyl]amino.

In one aspect, in the compounds of formula (IBi), one of R^{4a} and R^{4b} is hydrogen and the remaining R^{4a} or R^{4b} is selected from hydroxy, cyano, halogeno, formyl, carboxy, (C1-C6)alkyl, (C2-C6)alkenyl, (C2-C6)alkynyl, (C1-C6)alkoxy, (C2-C6)alkanoyl, (C1-C6)alkoxycarbonyl, (C3-C8)cycloalkyl, (C3-C8)cycloalkylcarbonyl, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (C1-C6)alkoxyamino, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, sulfamoyl, (C1-C6)alkylsulfamoyl, di-[(C1-C6)alkyl]sulfamoyl, —S(O)ₘR′ wherein R′ is as defined above and m represents 0, 1 or 2, —N(R″)C(O)R′ wherein R′ and R″ are each as defined above, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,

wherein each of the groups or rings within R^{4a} and R^{4b} may be optionally substituted by one or more substituents independently selected from hydroxy, halogeno, cyano, formyl, carboxy, (C1-C6)alkyl, (C3-C8)cycloalkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, (C1-C6)alkylthio, sulfamoyl, (C1-C6)alkylsulfamoyl, di-[(C1-C6)alkyl]sulfamoyl, —S(O)ₘR′ wherein R′ and m are each as defined above, —N(R″)C(O)R′ wherein R′ and R″ are each as defined above, and —X-Q wherein X and Q are each as defined above, any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ and m are each as defined above, —N(R″)C(O)R′ wherein R′ and R″ are each as defined above, and —X-Q wherein X and Q are each as defined above;

and wherein any saturated monocyclic ring optionally bears 1 or 2 oxo or thioxo substituents.

In another aspect, in the compounds of formula (IBi), one of R^{4a} and R^{4b} is hydrogen and the remaining R^{4a} or R^{4b} is —X-Q wherein X is a direct bond and Q represents a saturated 4-, 5-, 6-, 7-, 8-, 9- or 10-membered heterocyclic ring comprising at least one ring nitrogen atom and optionally comprising a further one or two ring heteroatoms selected from nitrogen, oxygen and sulfur,

each of which rings within R^{4a} or R^{4b} may be optionally substituted by one or more substituents independently selected from (C1-C6)alkyl and (2-6C)alkanoyl, any of which substituents may be optionally substituted by one or more further substituents independently selected from (C1-C4)alkyl, (C1-C4)alkoxy, (C1-C4)alkylamino, di-[(C1-C4)alkyl]amino, hydroxy and hydroxy(C1-C4)alkyl,

and wherein any saturated monocyclic ring optionally bears 1 or 2 oxo substituents.

In another aspect, in the compounds of formula (IBi), one of R^{4a} and R^{4b} is hydrogen and the remaining R^{4a} or R^{4b} is —X-Q wherein X is a direct bond and Q represents a piperazin-1-yl which may be optionally substituted by one or more substituents independently selected from (C1-C6)alkyl and (2-6C)alkanoyl, any of which substituents may be optionally substituted by one or more further substituents independently selected from (C1-C4)alkyl, (C1-C4)alkoxy, (C1-C4)alkylamino, di-[(C1-C4)alkyl]amino, hydroxy and hydroxy(C1-C4)alkyl,

and wherein any saturated monocyclic ring optionally bears 1 or 2 oxo substituents.

Particular compounds of the invention include, for example, any one or more compounds of formula (I) selected from:
1-[4-[4-[(5-chloro-4-imidazo[1,2-a]pyridin-3-yl-pyrimidin-2-yl)amino]-3-methoxy-phenyl]piperazin-1-yl]ethanone;
5-chloro-4-imidazo[1,2-a]pyridin-3-yl-N-(2-methoxy-4-piperazin-1-yl-phenyl)pyrimidin-2-amine;
[4-[(5-chloro-4-imidazo[1,2-a]pyridin-3-yl-pyrimidin-2-yl)amino]-3-methoxy-phenyl]-morpholino-methanone;
[1-[4-[(5-chloro-4-imidazo[1,2-a]pyridin-3-yl-pyrimidin-2-yl)amino]-3-methoxy-phenyl]pyrrolidin-2-yl]-morpholino-methanone;
2-[4-[4-[(5-chloro-4-imidazo[1,2-a]pyridin-3-yl-pyrimidin-2-yl)amino]-3-methoxy-phenyl]piperazin-1-yl]-1-morpholino-ethanone;
[3-[(5-chloro-4-imidazo[1,2-a]pyridin-3-yl-pyrimidin-2-yl)amino]-4-methoxy-phenyl]-morpholino-methanone;
(3S)-1-[3-[(5-chloro-4-imidazo[1,2-a]pyridin-3-yl-pyrimidin-2-yl)amino]-4-methoxy-phenyl]pyrrolidin-3-ol;
5-chloro-N-[4-(3,5-dimethylpiperazin-1-yl)-2-methoxy-phenyl]-4-imidazo[1,2-a]pyridin-3-yl-pyrimidin-2-amine;
4-[4-[(5-chloro-4-imidazo[1,2-a]pyridin-3-yl-pyrimidin-2-yl)amino]-3-methoxy-phenyl]-N,N-dimethyl-piperazine-1-sulfonamide;
5-chloro-4-imidazo[1,2-a]pyridin-3-yl-N-(2-methoxy-5-pyrrolidin-1-yl-phenyl)pyrimidin-2-amine;
1-[4-[3-[(5-chloro-4-imidazo[1,2-a]pyridin-3-yl-pyrimidin-2-yl)amino]-4-methoxy-phenyl]piperazin-1-yl]ethanone;
5-chloro-4-imidazo[1,2-a]pyridin-3-yl-N-(2-methoxy-5-piperazin-1-yl-phenyl)pyrimidin-2-amine;
5-chloro-N-[4-[(3R)-3-(dimethylamino)pyrrolidin-1-yl]-2-methoxy-phenyl]-4-imidazo[1,2-a]pyridin-3-yl-pyrimidin-2-amine;
5-chloro-4-imidazo[1,2-a]pyridin-3-yl-N-[2-methoxy-4-[2-[4-(2-methoxyethyl)piperazin-1-yl]ethoxy]phenyl]pyrimidin-2-amine;
5-chloro-N-[5-[(3R)-3-(dimethylamino)pyrrolidin-1-yl]-2-methoxy-phenyl]-4-imidazo[1,2-a]pyridin-3-yl-pyrimidin-2-amine;
5-chloro-N-[5-[(3S)-3-(dimethylamino)pyrrolidin-1-yl]-2-methyl-phenyl]-4-imidazo[1,2-a]pyridin-3-yl-pyrimidin-2-amine;
5-chloro-N-[4-(1,1-dioxo-1,4-thiazinan-4-yl)-2-methoxy-phenyl]-4-imidazo[1,2-a]pyridin-3-yl-pyrimidin-2-amine;
5-chloro-N-[4-[(3S)-3-(dimethylamino)pyrrolidin-1-yl]-2-methoxy-phenyl]-4-imidazo[1,2-a]pyridin-3-yl-pyrimidin-2-amine;
5-chloro-N-[5-[(3S)-3-(dimethylamino)pyrrolidin-1-yl]-2-methoxy-phenyl]-4-imidazo[1,2-a]pyridin-3-yl-pyrimidin-2-amine;
(3R)-1-[3-[(5-chloro-4-imidazo[1,2-a]pyridin-3-yl-pyrimidin-2-yl)amino]-4-methoxy-phenyl]pyrrolidin-3-ol;
5-chloro-4-imidazo[1,2-a]pyridin-3-yl-N-(2-methoxy-5-morpholino-phenyl)pyrimidin-2-amine;
5-chloro-4-imidazo[1,2-a]pyridin-3-yl-N-[2-methoxy-5-(4-methoxy-1-piperidyl)phenyl]pyrimidin-2-amine;
1-[4-[4-[(5-chloro-4-imidazo[1,2-a]pyridin-3-yl-pyrimidin-2-yl)amino]-3-methoxy-phenyl]-1-piperidyl]ethanone;
5-chloro-4-imidazo[1,2-a]pyridin-3-yl-N-[2-methoxy-4-(4-methylsulfonylpiperazin-1-yl)phenyl]pyrimidin-2-amine;
N-[(3R)-1-[4-[(5-chloro-4-imidazo[1,2-a]pyridin-3-yl-pyrimidin-2-yl)amino]-3-methoxy-phenyl]pyrrolidin-3-yl]acetamide;
4-[4-[(5-chloro-4-imidazo[1,2-a]pyridin-3-yl-pyrimidin-2-yl)amino]-3-methoxy-phenyl]pyrrolidin-2-one;
1-[2-[4-[(5-chloro-4-imidazo[1,2-a]pyridin-3-yl-pyrimidin-2-yl)amino]-3-methoxy-phenyl]-1-piperidyl]ethanone;
1-[4-[4-[(5-chloro-4-imidazo[1,2-a]pyridin-3-yl-pyrimidin-2-yl)amino]-3-fluoro-5-methoxy-phenyl]piperazin-1-yl]ethanone;
4-[4-[(5-chloro-4-imidazo[1,2-a]pyridin-3-yl-pyrimidin-2-yl)amino]-3-methoxy-phenyl]-N-methyl-pyrrolidine-2-carboxamide;
1-[4-[4-[(5-chloro-4-imidazo[1,2-a]pyridin-3-yl-pyrimidin-2-yl)amino]-3-methoxy-phenyl]-1,4-diazepan-1-yl]ethanone;
1-[3-[4-[(5-chloro-4-imidazo[1,2-a]pyridin-3-yl-pyrimidin-2-yl)amino]-3-methoxy-phenyl]-1-piperidyl]ethanone;
1-[4-[4-[(5-chloro-4-imidazo[1,2-a]pyridin-3-yl-pyrimidin-2-yl)amino]-3-methyl-phenyl]piperazin-1-yl]ethanone;
1-[4-[4-[(5-chloro-4-imidazo[1,2-a]pyridin-3-yl-pyrimidin-2-yl)amino]-3-methoxy-phenyl]-2,6-dimethyl-piperazin-1-yl]ethanone;
1-[4-[4-[(5-chloro-4-imidazo[1,2-a]pyridin-3-yl-pyrimidin-2-yl)amino]-2,3-dimethyl-phenyl]piperazin-1-yl]ethanone;
1-[4-[4-[(5-chloro-4-imidazo[1,2-a]pyridin-3-yl-pyrimidin-2-yl)amino]-2-fluoro-5-methyl-phenyl]piperazin-1-yl]ethanone;
1-[4-[4-[(5-chloro-4-imidazo[1,2-a]pyridin-3-yl-pyrimidin-2-yl)amino]-3-isopropoxy-phenyl]piperazin-1-yl]ethanone;
1-[(2S,5R)-4-[4-[(5-chloro-4-imidazo[1,2-a]pyridin-3-yl-pyrimidin-2-yl)amino]-3-methoxy-phenyl]-2,5-dimethyl-piperazin-1-yl]ethanone;
1-[4-[4-[(5-chloro-4-imidazo[1,2-a]pyridin-3-yl-pyrimidin-2-yl)amino]-3-methoxy-phenyl]-2-methyl-piperazin-1-yl]ethanone;
1-[4-[3-[(5-chloro-4-imidazo[1,2-a]pyridin-3-yl-pyrimidin-2-yl)amino]-2-methyl-phenyl]piperazin-1-yl]ethanone;
1-[4-[4-[(5-chloro-4-imidazo[1,2-a]pyridin-3-yl-pyrimidin-2-yl)amino]-3-methoxy-phenyl]-2-(hydroxymethyl)piperazin-1-yl]ethanone;
2-[4-[(5-chloro-4-imidazo[1,2-a]pyridin-3-yl-pyrimidin-2-yl)amino]-3-methoxy-phenyl]-1-morpholino-propan-1-one;
1-[(6S)-4-[4-[(5-chloro-4-imidazo[1,2-a]pyridin-3-yl-pyrimidin-2-yl)amino]-3-methoxy-phenyl]-6-hydroxy-1,4-diazepan-1-yl]ethanone;
2-[4-[(5-chloro-4-imidazo[1,2-a]pyridin-3-yl-pyrimidin-2-yl)amino]-3-methoxy-phenyl]-1-[4-(2-hydroxyethyl)piperazin-1-yl]propan-1-one;
2-[4-[(5-chloro-4-imidazo[1,2-a]pyridin-3-yl-pyrimidin-2-yl)amino]-3-methoxy-phenyl]-1-piperazin-1-yl-propan-1-one;
5-chloro-4-imidazo[1,2-a]pyridin-3-yl-N-(2-methoxy-4-morpholino-phenyl)pyrimidin-2-amine;
5-chloro-4-imidazo[1,2-a]pyridin-3-yl-N-(2-methoxy-4-pyrrolidin-1-yl-phenyl)pyrimidin-2-amine;
2-[4-[4-[(5-chloro-4-imidazo[1,2-a]pyridin-3-yl-pyrimidin-2-yl)amino]-3-methoxy-phenyl]piperazin-1-yl]ethanol;
5-chloro-4-imidazo[1,2-a]pyridin-3-yl-N-[2-methoxy-4-[4-(2-methoxyethyl)piperazin-1-yl]phenyl]pyrimidin-2-amine;
(3S)-1-[4-[(5-chloro-4-imidazo[1,2-a]pyridin-3-yl-pyrimidin-2-yl)amino]-3-methoxy-phenyl]pyrrolidin-3-ol;
5-chloro-4-imidazo[1,2-a]pyridin-3-yl-N-[2-methoxy-4-(1,4-oxazepan-4-yl)phenyl]pyrimidin-2-amine;
(3R)-1-[4-[(5-chloro-4-imidazo[1,2-a]pyridin-3-yl-pyrimidin-2-yl)amino]-3-methoxy-phenyl]pyrrolidin-3-ol;
5-chloro-4-imidazo[1,2-a]pyridin-3-yl-N-[2-methoxy-4-(4-piperidyl)phenyl]pyrimidin-2-amine;
5-chloro-4-imidazo[1,2-a]pyridin-3-yl-N-[2-methoxy-4-(4-piperidyloxy)phenyl]pyrimidin-2-amine;
5-chloro-4-imidazo[1,2-a]pyridin-3-yl-N-[2-methoxy-4-[[(2R)-pyrrolidin-2-yl]methoxy]phenyl]pyrimidin-2-amine;
5-chloro-4-imidazo[1,2-a]pyridin-3-yl-N-[2-methoxy-4-[[(2S)-pyrrolidin-2-yl]methoxy]phenyl]pyrimidin-2-amine;
5-chloro-4-imidazo[1,2-a]pyridin-3-yl-N-[2-methoxy-5-[[(2S)-pyrrolidin-2-yl]methoxy]phenyl]pyrimidin-2-amine;
5-chloro-4-imidazo[1,2-a]pyridin-3-yl-N-[2-methoxy-5-[[(2R)-pyrrolidin-2-yl]methoxy]phenyl]pyrimidin-2-amine;
5-chloro-4-imidazo[1,2-a]pyridin-3-yl-N-[2-methoxy-4-[(3R)-pyrrolidin-3-yl]oxy-phenyl]pyrimidin-2-amine;
5-chloro-4-imidazo[1,2-a]pyridin-3-yl-N-[2-methoxy-4-[(3S)-pyrrolidin-3-yl]oxy-phenyl]pyrimidin-2-amine;
1-[4-[4-[(5-chloro-4-imidazo[1,2-a]pyridin-3-yl-pyrimidin-2-yl)amino]-3-methoxy-phenoxy]-1-piperidyl]ethanone;
5-chloro-4-imidazo[1,2-a]pyridin-3-yl-N-[2-methoxy-4-[(1-methylsulfonyl-4-piperidyl)oxy]phenyl]pyrimidin-2-amine;
(2S)-1-[4-[4-[(5-chloro-4-imidazo[1,2-a]pyridin-3-yl-pyrimidin-2-yl)amino]-3-methoxy-phenoxy]-1-piperidyl]-2-hydroxy-propan-1-one;
1-[4-[4-[(5-chloro-4-imidazo[1,2-a]pyridin-3-yl-pyrimidin-2-yl)amino]-3-methoxy-phenoxy]-1-piperidyl]-2-hydroxy-propan-1-one;
1-[(3R)-3-[4-[(5-chloro-4-imidazo[1,2-a]pyridin-3-yl-pyrimidin-2-yl)amino]-3-methoxy-phenoxy]pyrrolidin-1-yl]ethanone;
5-chloro-4-imidazo[1,2-a]pyridin-3-yl-N-[2-methoxy-4-[(3R)-1-methylsulfonylpyrrolidin-3-yl]oxy-phenyl]pyrimidin-2-amine;
1-[(3R)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one;
(2S)-1-[(3R)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one;
1-[(3S)-3-[4-[(5-chloro-4-imidazo[1,2-a]pyridin-3-yl-pyrimidin-2-yl)amino]-3-methoxy-phenoxy]pyrrolidin-1-yl]ethanone;
5-chloro-4-imidazo[1,2-a]pyridin-3-yl-N-[2-methoxy-4-[(3S)-1-methylsulfonylpyrrolidin-3-yl]oxy-phenyl]pyrimidin-2-amine;
(2S)-1-[(3S)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one;
1-[(3S)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one;
1-[(2S)-2-[[4-[(5-chloro-4-imidazo[1,2-a]pyridin-3-yl-pyrimidin-2-yl)amino]-3-methoxy-phenoxy]methyl]pyrrolidin-1-yl]ethanone;
1-[(2R)-2-[[3-[(5-chloro-4-imidazo[1,2-a]pyridin-3-yl-pyrimidin-2-yl)amino]-4-methoxy-phenoxy]methyl]pyrrolidin-1-yl]ethanone;
4-[4-[(5-chloro-4-imidazo[1,2-a]pyridin-3-yl-pyrimidin-2-yl)amino]-3-methoxy-phenyl]piperazin-2-one;
4-[4-[(5-chloro-4-imidazo[1,2-a]pyridin-3-yl-pyrimidin-2-yl)amino]-3-methoxy-phenyl]-N,N-dimethyl-piperazine-2-carboxamide;
(3S)-1-[[4-[(5-chloro-4-imidazo[1,2-a]pyridin-3-yl-pyrimidin-2-yl)amino]-3-methoxy-phenyl]methyl]pyrrolidin-3-ol;
2-[[4-[(5-chloro-4-imidazo[1,2-a]pyridin-3-yl-pyrimidin-2-yl)amino]-3-methoxy-phenyl]methylamino]ethanol;
2-[[4-[(5-chloro-4-imidazo[1,2-a]pyridin-3-yl-pyrimidin-2-yl)amino]-3-methoxy-phenyl]methyl-methyl-amino]ethanol;
3-[[4-[(5-chloro-4-imidazo[1,2-a]pyridin-3-yl-pyrimidin-2-yl)amino]-3-methoxy-phenyl]methylamino]propan-1-ol;
5-chloro-4-imidazo[1,2-a]pyridin-3-yl-N-[2-methoxy-4-[(4-methylpiperazin-1-yl)methyl]phenyl]pyrimidin-2-amine;
1-[[4-[(5-chloro-4-imidazo[1,2-a]pyridin-3-yl-pyrimidin-2-yl)amino]-3-methoxy-phenyl]methyl]piperidin-4-ol;
1-[4-[[4-[(5-chloro-4-imidazo[1,2-a]pyridin-3-yl-pyrimidin-2-yl)amino]-3-methoxy-phenyl]methyl]piperazin-1-yl]ethanone;
1-[[4-[(5-chloro-4-imidazo[1,2-a]pyridin-3-yl-pyrimidin-2-yl)amino]-3-methoxy-phenyl]methyl]piperidine-4-carboxamide;
2-[4-[[4-[(5-chloro-4-imidazo[1,2-a]pyridin-3-yl-pyrimidin-2-yl)amino]-3-methoxy-phenyl]methyl]piperazin-1-yl]ethanol;
5-chloro-4-imidazo[1,2-a]pyridin-3-yl-N-[2-methoxy-4-[(3-methoxypropylamino)methyl]phenyl]pyrimidin-2-amine;
N-[4-(azetidin-1-ylmethyl)-2-methoxy-phenyl]-5-chloro-4-imidazo[1,2-a]pyridin-3-yl-pyrimidin-2-amine;
5-chloro-4-imidazo[1,2-a]pyridin-3-yl-N-[2-methoxy-4-[(4-methyl-1,4-diazepan-1-yl)methyl]phenyl]pyrimidin-2-amine;
(3R)-1-[[4-[(5-chloro-4-imidazo[1,2-a]pyridin-3-yl-pyrimidin-2-yl)amino]-3-methoxy-phenyl]methyl]piperidin-3-ol;
5-chloro-4-imidazo[1,2-a]pyridin-3-yl-N-[2-methoxy-4-[[2-methoxyethyl(methyl)amino]methyl]phenyl]pyrimidin-2-amine;
5-chloro-4-imidazo[1,2-a]pyridin-3-yl-N-[2-methoxy-4-(1,4-oxazepan-4-ylmethyl)phenyl]pyrimidin-2-amine;
(2R)-2-[[4-[(5-chloro-4-imidazo[1,2-a]pyridin-3-yl-pyrimidin-2-yl)amino]-3-methoxy-phenyl]methylamino]propan-1-ol;
1-[[4-[(5-chloro-4-imidazo[1,2-a]pyridin-3-yl-pyrimidin-2-yl)amino]-3-methoxy-phenyl]methyl]azetidin-3-ol;
(3S)-1-[[4-[(5-chloro-4-imidazo[1,2-a]pyridin-3-yl-pyrimidin-2-yl)amino]-3-methoxy-phenyl]methyl]piperidin-3-ol;
(3R)-1-[[4-[(5-chloro-4-imidazo[1,2-a]pyridin-3-yl-pyrimidin-2-yl)amino]-3-methoxy-phenyl]methyl]pyrrolidin-3-ol;
(2S)-2-[[4-[(5-chloro-4-imidazo[1,2-a]pyridin-3-yl-pyrimidin-2-yl)amino]-3-methoxy-phenyl]methylamino]propan-1-ol;
5-chloro-4-imidazo[1,2-a]pyridin-3-yl-N-[2-methoxy-4-(morpholinomethyl)phenyl]pyrimidin-2-amine;
[4-[4-[(5-chloro-4-imidazo[1,2-a]pyridin-3-yl-pyrimidin-2-yl)amino]-3-methoxy-phenyl]piperazin-1-yl]-(1-hydroxycyclopropyl)methanone;
[4-[4-[(5-chloro-4-imidazo[1,2-a]pyridin-3-yl-pyrimidin-2-yl)amino]-3-methoxy-phenyl]piperazin-1-yl]-[(2S)-pyrrolidin-2-yl]methanone;
[4-[4-[(5-chloro-4-imidazo[1,2-a]pyridin-3-yl-pyrimidin-2-yl)amino]-3-methoxy-phenyl]piperazin-1-yl]-[(2R)-2-piperidyl]methanone;
[4-[4-[(5-chloro-4-imidazo[1,2-a]pyridin-3-yl-pyrimidin-2-yl)amino]-3-methoxy-phenyl]piperazin-1-yl]-[(2S)-2-piperidyl]methanone;
2-amino-1-[4-[4-[(5-chloro-4-imidazo[1,2-a]pyridin-3-yl-pyrimidin-2-yl)amino]-3-methoxy-phenyl]piperazin-1-yl]ethanone;
(2S)-2-amino-1-[4-[4-[(5-chloro-4-imidazo[1,2-a]pyridin-3-yl-pyrimidin-2-yl)amino]-3-methoxy-phenyl]piperazin-1-yl]propan-1-one;
(2S)-2-amino-1-[4-[4-[(5-chloro-4-imidazo[1,2-a]pyridin-3-yl-pyrimidin-2-yl)amino]-3-methoxy-phenyl]piperazin-1-yl]-3-methyl-butan-1-one;
(2S)-2-amino-1-[4-[4-[(5-chloro-4-imidazo[1,2-a]pyridin-3-yl-pyrimidin-2-yl)amino]-3-methoxy-phenyl]piperazin-1-yl]-3-hydroxy-propan-1-one;
(3S)-3-amino-4-[4-[4-[(5-chloro-4-imidazo[1,2-a]pyridin-3-yl-pyrimidin-2-yl)amino]-3-methoxy-phenyl]piperazin-1-yl]-4-oxo-butanamide;
[4-[4-[(5-chloro-4-imidazo[1,2-a]pyridin-3-yl-pyrimidin-2-yl)amino]-3-methoxy-phenyl]piperazin-1-yl]-[(2S,4R)-4-hydroxypyrrolidin-2-yl]methanone;
1-[4-[4-[(5-chloro-4-imidazo[1,2-a]pyridin-3-yl-pyrimidin-2-yl)amino]-3-methoxy-phenyl]piperazin-1-yl]-2-(methylamino)ethanone;
1-[4-[4-[(5-chloro-4-imidazo[1,2-a]pyridin-3-yl-pyrimidin-2-yl)amino]-3-methoxy-phenyl]piperazin-1-yl]-2-(ethylamino)ethanone;
(2R)-2-amino-1-[4-[4-[(5-chloro-4-imidazo[1,2-a]pyridin-3-yl-pyrimidin-2-yl)amino]-3-methoxy-phenyl]piperazin-1-yl]-3-hydroxy-propan-1-one;
(2S)-2-amino-1-[4-[4-[(5-chloro-4-imidazo[1,2-a]pyridin-3-yl-pyrimidin-2-yl)amino]-3-methoxy-phenyl]piperazin-1-yl]-4-hydroxy-butan-1-one;
(2R)-2-amino-1-[4-[4-[(5-chloro-4-imidazo[1,2-a]pyridin-3-yl-pyrimidin-2-yl)amino]-3-methoxy-phenyl]piperazin-1-yl]propan-1-one;
5-[4-[4-[(5-chloro-4-imidazo[1,2-a]pyridin-3-yl-pyrimidin-2-yl)amino]-3-methoxy-phenyl]piperazine-1-carbonyl]pyrrolidin-2-one;
(5S)-5-[4-[4-[(5-chloro-4-imidazo[1,2-a]pyridin-3-yl-pyrimidin-2-yl)amino]-3-methoxy-phenyl]piperazine-1-carbonyl]pyrrolidin-2-one;
N-[2-[4-[4-[(5-chloro-4-imidazo[1,2-a]pyridin-3-yl-pyrimidin-2-yl)amino]-3-methoxy-phenyl]piperazin-1-yl]-2-oxo-ethyl]acetamide;
(2S)-1-[4-[4-[(5-chloro-4-imidazo[1,2-a]pyridin-3-yl-pyrimidin-2-yl)amino]-3-methoxy-phenyl]piperazin-1-yl]-2-methoxy-propan-1-one;
(2R)-1-[4-[4-[(5-chloro-4-imidazo[1,2-a]pyridin-3-yl-pyrimidin-2-yl)amino]-3-methoxy-phenyl]piperazin-1-yl]-2-hydroxy-propan-1-one;
(2R)-1-[4-[4-[(5-chloro-4-imidazo[1,2-a]pyridin-3-yl-pyrimidin-2-yl)amino]-3-methoxy-phenyl]piperazin-1-yl]-2-methoxy-propan-1-one;
1-[4-[4-[(5-chloro-4-imidazo[1,2-a]pyridin-3-yl-pyrimidin-2-yl)amino]-3-methoxy-phenyl]piperazin-1-yl]-2-hydroxy-2-methyl-propan-1-one;
N-[4-(azetidin-3-yloxy)-2-methoxy-phenyl]-5-chloro-4-imidazo[1,2-a]pyridin-3-yl-pyrimidin-2-amine;
(2S)-1-[3-[4-[(5-chloro-4-imidazo[1,2-a]pyridin-3-yl-pyrimidin-2-yl)amino]-3-methoxy-phenoxy]azetidin-1-yl]-2-hydroxy-propan-1-one;
(2S)-1-[3-[4-[(5-chloro-4-imidazo[1,2-a]pyridin-3-yl-pyrimidin-2-yl)amino]-3-methoxy-phenoxy]azetidin-1-yl]-2-methoxy-propan-1-one;
1-[3-[4-[(5-chloro-4-imidazo[1,2-a]pyridin-3-yl-pyrimidin-2-yl)amino]-3-methoxy-phenoxy]azetidin-1-yl]-2-hydroxy-ethanone;
(2R)-1-[3-[4-[(5-chloro-4-imidazo[1,2-a]pyridin-3-yl-pyrimidin-2-yl)amino]-3-methoxy-phenoxy]azetidin-1-yl]-2-methoxy-propan-1-one;
[3-[4-[(5-chloro-4-imidazo[1,2-a]pyridin-3-yl-pyrimidin-2-yl)amino]-3-methoxy-phenoxy]azetidin-1-yl]-(1-hydroxycyclopropyl)methanone;
(2R)-1-[3-[4-[(5-chloro-4-imidazo[1,2-a]pyridin-3-yl-pyrimidin-2-yl)amino]-3-methoxy-phenoxy]azetidin-1-yl]-2-hydroxy-propan-1-one;
(2R)-1-[4-[4-[(5-chloro-4-imidazo[1,2-a]pyridin-3-yl-pyrimidin-2-yl)amino]-3-methoxy-phenyl]-1-piperidyl]-2-methoxy-propan-1-one;
(2R)-1-[4-[4-[(5-chloro-4-imidazo[1,2-a]pyridin-3-yl-pyrimidin-2-yl)amino]-3-methoxy-phenyl]-1-piperidyl]-2-hydroxy-propan-1-one;
(2S)-1-[4-[4-[(5-chloro-4-imidazo[1,2-a]pyridin-3-yl-pyrimidin-2-yl)amino]-3-methoxy-phenyl]-1-piperidyl]-2-methoxy-propan-1-one;
(2S)-1-[4-[4-[(5-chloro-4-imidazo[1,2-a]pyridin-3-yl-pyrimidin-2-yl)amino]-3-methoxy-phenyl]-1-piperidyl]-2-hydroxy-propan-1-one;
1-[4-[4-[(5-chloro-4-imidazo[1,2-a]pyridin-3-yl-pyrimidin-2-yl)amino]-3-methoxy-phenyl]-1-piperidyl]-2-hydroxy-ethanone;
[4-[(5-chloro-4-imidazo[1,2-a]pyridin-3-yl-pyrimidin-2-yl)amino]-3-methoxy-phenyl]-[(1S,4S)-3,6-diazabicyclo[2.2.1]heptan-6-yl]methanone;
[4-[(5-chloro-4-imidazo[1,2-a]pyridin-3-yl-pyrimidin-2-yl)amino]-3-methoxy-phenyl]-piperazin-1-yl-methanone;
[4-[(5-chloro-4-imidazo[1,2-a]pyridin-3-yl-pyrimidin-2-yl)amino]-3-methoxy-phenyl]-[(1S,4S)-5-methyl-2,5-diazabicyclo[2.2.1]heptan-2-yl]methanone;
[4-[(5-chloro-4-imidazo[1,2-a]pyridin-3-yl-pyrimidin-2-yl)amino]-3-methoxy-phenyl]-[4-(2-hydroxyethyl)piperazin-1-yl]methanone;
4-[(5-chloro-4-imidazo[1,2-a]pyridin-3-yl-pyrimidin-2-yl)amino]-N-(2-hydroxyethyl)-3-methoxy-N-methyl-benzamide;
2-[4-[(5-chloro-4-imidazo[1,2-a]pyridin-3-yl-pyrimidin-2-yl)amino]-3-methoxy-phenyl]-1-piperazin-1-yl-ethanone;
2-[4-[(5-chloro-4-imidazo[1,2-a]pyridin-3-yl-pyrimidin-2-yl)amino]-3-methoxy-phenyl]-N-pyrrolidin-3-yl-acetamide;
2-[4-[(5-chloro-4-imidazo[1,2-a]pyridin-3-yl-pyrimidin-2-yl)amino]-3-methoxy-phenyl]-1-morpholino-ethanone;
2-[4-[(5-chloro-4-imidazo[1,2-a]pyridin-3-yl-pyrimidin-2-yl)amino]-3-methoxy-phenyl]-1-[4-(2-hydroxyethyl)piperazin-1-yl]ethanone;
2-[4-[(5-chloro-4-imidazo[1,2-a]pyridin-3-yl-pyrimidin-2-yl)amino]-3-methoxy-phenyl]-1-[(3R)-3-hydroxypyrrolidin-1-yl]ethanone;
1-[4-[4-[(5-chloro-4-imidazo[1,2-a]pyridin-3-yl-pyrimidin-2-yl)amino]-3-methoxy-phenyl]piperazin-1-yl]-2-methoxy-ethanone;
1-[4-[4-[(5-chloro-4-imidazo[1,2-a]pyridin-3-yl-pyrimidin-2-yl)amino]-3-methoxy-phenyl]piperazin-1-yl]-2-hydroxy-ethanone;
(2S)-1-[4-[4-[(5-chloro-4-imidazo[1,2-a]pyridin-3-yl-pyrimidin-2-yl)amino]-3-methoxy-phenyl]piperazin-1-yl]-2-hydroxy-propan-1-one;
1-[4-[4-[(5-chloro-4-imidazo[1,2-a]pyridin-3-yl-pyrimidin-2-yl)amino]-3-methoxy-phenyl]piperazin-1-yl]propan-1-one;
methyl 4-[4-[(5-chloro-4-imidazo[1,2-a]pyridin-3-yl-pyrimidin-2-yl)amino]-3-methoxy-phenyl]piperazine-1-carboxylate;
2-methoxyethyl 4-[4-[(5-chloro-4-imidazo[1,2-a]pyridin-3-yl-pyrimidin-2-yl)amino]-3-methoxy-phenyl]piperazine-1-carboxylate;
[4-[4-[(5-chloro-4-imidazo[1,2-a]pyridin-3-yl-pyrimidin-2-yl)amino]-3-methoxy-phenyl]piperazin-1-yl]-morpholino-methanone;
4-[4-[(5-chloro-4-imidazo[1,2-a]pyridin-3-yl-pyrimidin-2-yl)amino]-3-methoxy-phenyl]-N-(2-hydroxyethyl)-N-methyl-piperazine-1-carboxamide;
4-[4-[(5-chloro-4-imidazo[1,2-a]pyridin-3-yl-pyrimidin-2-yl)amino]-3-methoxy-phenyl]-N,N-dimethyl-piperazine-1-carboxamide;
[4-[4-[(5-chloro-4-imidazo[1,2-a]pyridin-3-yl-pyrimidin-2-yl)amino]-3-methoxy-phenyl]piperazin-1-yl]-piperazin-1-yl-methanone;
5-chloro-4-imidazo[1,2-a]pyridin-3-yl-N-[2-methoxy-4-[4-(2-methylsulfonylethyl)piperazin-1-yl]phenyl]pyrimidin-2-amine;
3-[4-[4-[(5-chloro-4-imidazo[1,2-a]pyridin-3-yl-pyrimidin-2-yl)amino]-3-methoxy-phenyl]piperazin-1-yl]propanenitrile;
(2S)-1-[4-[4-[(5-chloro-4-imidazo[1,2-a]pyridin-3-yl-pyrimidin-2-yl)amino]-3-methoxy-phenyl]piperazin-1-yl]propan-2-ol;
(2R)-1-[4-[4-[(5-chloro-4-imidazo[1,2-a]pyridin-3-yl-pyrimidin-2-yl)amino]-3-methoxy-phenyl]piperazin-1-yl]propan-2-ol;
1-[4-[4-[(5-chloro-4-imidazo[1,2-a]pyridin-3-yl-pyrimidin-2-yl)amino]-3-methoxy-phenyl]piperazin-1-yl]-2-methyl-propan-2-ol;
(2R)-3-[4-[4-[(5-chloro-4-imidazo[1,2-a]pyridin-3-yl-pyrimidin-2-yl)amino]-3-methoxy-phenyl]piperazin-1-yl]propane-1,2-diol;
4-[4-[(5-chloro-4-imidazo[1,2-a]pyridin-3-yl-pyrimidin-2-yl)amino]-3-methoxy-phenyl]piperazine-1-carboxamide;
4-[3-[(5-chloro-4-imidazo[1,2-a]pyridin-3-yl-pyrimidin-2-yl)amino]-4-methoxy-phenyl]piperazine-1-carboxamide;
(2S)-1-[4-[4-[(5-chloro-4-imidazo[1,2-a]pyridin-3-yl-pyrimidin-2-yl)amino]-3-methoxy-phenyl]piperazin-1-yl]-2,3-dihydroxy-propan-1-one;
4-[4-[(5-chloro-4-imidazo[1,2-a]pyridin-3-yl-pyrimidin-2-yl)amino]-3-methoxy-phenyl]-N,N-dimethyl-piperidine-1-carboxamide;
4-[4-[(5-chloro-4-imidazo[1,2-a]pyridin-3-yl-pyrimidin-2-yl)amino]-3-methoxy-phenyl]-N-(2-hydroxyethyl)-N-methyl-piperidine-1-carboxamide;
1-[4-[4-[(5-chloro-4-imidazo[1,2-a]pyridin-3-yl-pyrimidin-2-yl)amino]-3-methoxy-phenyl]-2-methyl-1-piperidyl]ethanone;
5-chloro-4-imidazo[1,2-a]pyridin-3-yl-N-[2-methoxy-4-(2-methyl-4-piperidyl)phenyl]pyrimidin-2-amine;
1-[3-[4-[(5-chloro-4-imidazo[1,2-a]pyridin-3-yl-pyrimidin-2-yl)amino]-3-methoxy-phenoxy]azetidin-1-yl]ethanone;
(2S)-1-[3-[4-[(5-chloro-4-imidazo[1,2-a]pyridin-3-yl-pyrimidin-2-yl)amino]-3-methoxy-phenoxy]azetidin-1-yl]propan-2-ol;
(2R)-1-[3-[4-[(5-chloro-4-imidazo[1,2-a]pyridin-3-yl-pyrimidin-2-yl)amino]-3-methoxy-phenoxy]azetidin-1-yl]propan-2-ol;
1-[3-[4-[(5-chloro-4-imidazo[1,2-a]pyridin-3-yl-pyrimidin-2-yl)amino]-3-methoxy-phenoxy]azetidin-1-yl]-2-methyl-propan-2-ol;
2-[3-[4-[(5-chloro-4-imidazo[1,2-a]pyridin-3-yl-pyrimidin-2-yl)amino]-3-methoxy-phenoxy]azetidin-1-yl]ethanol;
2-[4-[4-[(5-chloro-4-imidazo[1,2-a]pyridin-3-yl-pyrimidin-2-yl)amino]-3-methoxy-phenyl]piperazin-1-yl]-N,N-dimethyl-acetamide;
2-[4-[4-[(5-chloro-4-imidazo[1,2-a]pyridin-3-yl-pyrimidin-2-yl)amino]-3-methoxy-phenyl]piperazin-1-yl]-N-methyl-acetamide;
2-[4-[4-[(5-chloro-4-imidazo[1,2-a]pyridin-3-yl-pyrimidin-2-yl)amino]-3-methoxy-phenyl]piperazin-1-yl]-N-(2-hydroxyethyl)-N-methyl-acetamide;
3-[4-[(5-chloro-4-imidazo[1,2-a]pyridin-3-yl-pyrimidin-2-yl)amino]-3-methoxy-phenoxy]-N-(2-hydroxyethyl)-N-methyl-azetidine-1-carboxamide;
3-[4-[(5-chloro-4-imidazo[1,2-a]pyridin-3-yl-pyrimidin-2-yl)amino]-3-methoxy-phenoxy]-N,N-dimethyl-azetidine-1-carboxamide;
2-[4-[(5-chloro-4-imidazo[1,2-a]pyridin-3-yl-pyrimidin-2-yl)amino]-3-methoxy-phenyl]propane-1,3-diol;
N-[4-[(3R)-3-aminopyrrolidin-1-yl]-2-methoxy-phenyl]-5-chloro-4-imidazo[1,2-a]pyridin-3-yl-pyrimidin-2-amine;
1-[4-[4-[(5-fluoro-4-imidazo[1,2-a]pyridin-3-yl-pyrimidin-2-yl)amino]-3-methoxy-phenyl]piperazin-1-yl]ethanone;
5-fluoro-4-imidazo[1,2-a]pyridin-3-yl-N-(2-methoxy-4-piperazin-1-yl-phenyl)pyrimidin-2-amine;
4-[4-[(5-fluoro-4-imidazo[1,2-a]pyridin-3-yl-pyrimidin-2-yl)amino]-3-methoxy-phenyl]-N,N-dimethyl-piperazine-1-sulfonamide;
5-fluoro-4-imidazo[1,2-a]pyridin-3-yl-N-(2-methoxy-5-piperazin-1-yl-phenyl)pyrimidin-2-amine;
N-[4-[(3R)-3-(dimethylamino)pyrrolidin-1-yl]-2-methoxy-phenyl]-5-fluoro-4-imidazo[1,2-a]pyridin-3-yl-pyrimidin-2-amine;
5-fluoro-4-imidazo[1,2-a]pyridin-3-yl-N-[2-methoxy-5-(4-methoxy-1-piperidyl)phenyl]pyrimidin-2-amine;
1-[4-[4-[(5-fluoro-4-imidazo[1,2-a]pyridin-3-yl-pyrimidin-2-yl)amino]-3-methoxy-phenyl]-1-piperidyl]ethanone;
5-fluoro-4-imidazo[1,2-a]pyridin-3-yl-N-(2-methoxy-4-morpholino-phenyl)pyrimidin-2-amine;
5-fluoro-4-imidazo[1,2-a]pyridin-3-yl-N-(2-methoxy-4-pyrrolidin-1-yl-phenyl)pyrimidin-2-amine;
2-[4-[4-[(5-fluoro-4-imidazo[1,2-a]pyridin-3-yl-pyrimidin-2-yl)amino]-3-methoxy-phenyl]piperazin-1-yl]ethanol;
5-fluoro-4-imidazo[1,2-a]pyridin-3-yl-N-[2-methoxy-4-[4-(2-methoxyethyl)piperazin-1-yl]phenyl]pyrimidin-2-amine;
(3S)-1-[4-[(5-fluoro-4-imidazo[1,2-a]pyridin-3-yl-pyrimidin-2-yl)amino]-3-methoxy-phenyl]pyrrolidin-3-ol;
5-fluoro-4-imidazo[1,2-a]pyridin-3-yl-N-[2-methoxy-4-(1,4-oxazepan-4-yl)phenyl]pyrimidin-2-amine;
(3R)-1-[4-[(5-fluoro-4-imidazo[1,2-a]pyridin-3-yl-pyrimidin-2-yl)amino]-3-methoxy-phenyl]pyrrolidin-3-ol;
5-fluoro-4-imidazo[1,2-a]pyridin-3-yl-N-[2-methoxy-4-(4-piperidyl)phenyl]pyrimidin-2-amine;
5-fluoro-4-imidazo[1,2-a]pyridin-3-yl-N-[2-methoxy-4-[[(2R)-pyrrolidin-2-yl]methoxy]phenyl]pyrimidin-2-amine;
4-[4-[(5-fluoro-4-imidazo[1,2-a]pyridin-3-yl-pyrimidin-2-yl)amino]-3-methoxy-phenyl]piperazin-2-one;
(2S)-1-[4-[4-[(5-fluoro-4-imidazo[1,2-a]pyridin-3-yl-pyrimidin-2-yl)amino]-3-methoxy-phenyl]piperazin-1-yl]propan-2-ol;
(2R)-1-[4-[4-[(5-fluoro-4-imidazo[1,2-a]pyridin-3-yl-pyrimidin-2-yl)amino]-3-methoxy-phenyl]piperazin-1-yl]propan-2-ol;
1-[4-[4-[(5-fluoro-4-imidazo[1,2-a]pyridin-3-yl-pyrimidin-2-yl)amino]-3-methoxy-phenyl]piperazin-1-yl]-2-methyl-propan-2-ol;
1-[4-[4-[(4-imidazo[1,2-a]pyridin-3-yl-5-methoxy-pyrimidin-2-yl)amino]-3-methoxy-phenyl]piperazin-1-yl]ethanone;
4-imidazo[1,2-a]pyridin-3-yl-5-methoxy-N-(2-methoxy-4-piperazin-1-yl-phenyl)pyrimidin-2-amine;
1-[4-[4-[(4-imidazo[1,2-a]pyridin-3-yl-5-methoxy-pyrimidin-2-yl)amino]-3-methoxy-phenyl]-1-piperidyl]ethanone;
2-[4-[4-[(4-imidazo[1,2-a]pyridin-3-yl-5-methoxy-pyrimidin-2-yl)amino]-3-methoxy-phenyl]piperazin-1-yl]ethanol;
4-imidazo[1,2-a]pyridin-3-yl-5-methoxy-N-[2-methoxy-4-[[(2R)-pyrrolidin-2-yl]methoxy]phenyl]pyrimidin-2-amine;
1-[4-[4-[(4-imidazo[1,2-a]pyridin-3-yl-5-methyl-pyrimidin-2-yl)amino]-3-methoxy-phenyl]piperazin-1-yl]ethanone;
4-imidazo[1,2-a]pyridin-3-yl-N-(2-methoxy-4-piperazin-1-yl-phenyl)-5-methyl-pyrimidin-2-amine;
1-[4-[4-[(4-imidazo[1,2-a]pyridin-3-yl-5-methyl-pyrimidin-2-yl)amino]-3-methoxy-phenyl]-1-piperidyl]ethanone;
4-imidazo[1,2-a]pyridin-3-yl-N-(2-methoxy-4-morpholino-phenyl)-5-methyl-pyrimidin-2-amine;
2-[4-[4-[(4-imidazo[1,2-a]pyridin-3-yl-5-methyl-pyrimidin-2-yl)amino]-3-methoxy-phenyl]piperazin-1-yl]ethanol;
4-imidazo[1,2-a]pyridin-3-yl-N-[2-methoxy-4-[[(2R)-pyrrolidin-2-yl]methoxy]phenyl]-5-methyl-pyrimidin-2-amine;
1-[4-[4-[(4-imidazo[1,2-a]pyridin-3-yl-5-methyl-pyrimidin-2-yl)amino]-3-methoxy-phenoxy]-1-piperidyl]ethanone;
(2S)-1-[4-[4-[(4-imidazo[1,2-a]pyridin-3-yl-5-methyl-pyrimidin-2-yl)amino]-3-methoxy-phenyl]piperazin-1-yl]propan-2-ol;
(2R)-1-[4-[4-[(4-imidazo[1,2-a]pyridin-3-yl-5-methyl-pyrimidin-2-yl)amino]-3-methoxy-phenyl]piperazin-1-yl]propan-2-ol;
1-[4-[4-[(4-imidazo[1,2-a]pyridin-3-yl-5-methyl-pyrimidin-2-yl)amino]-3-methoxy-phenyl]piperazin-1-yl]-2-methyl-propan-2-ol;
1-[4-[4-[[4-imidazo[1,2-a]pyridin-3-yl-5-(trifluoromethyl)pyrimidin-2-yl]amino]-3-methoxy-phenyl]piperazin-1-yl]ethanone;
1-[4-[4-[[4-imidazo[1,2-a]pyridin-3-yl-5-(trifluoromethyl)pyrimidin-2-yl]amino]-3-methoxy-phenyl]-1-piperidyl]ethanone;
2-[4-[4-[[4-imidazo[1,2-a]pyridin-3-yl-5-(trifluoromethyl)pyrimidin-2-yl]amino]-3-methoxy-phenyl]piperazin-1-yl]ethanol;
1-[4-[4-[[4-imidazo[1,2-a]pyridin-3-yl-5-(trifluoromethyl)pyrimidin-2-yl]amino]-3-methoxy-phenoxy]-1-piperidyl]ethanone;
1-[4-[(5-fluoro-4-imidazo[1,2-a]pyridin-3-yl-pyrimidin-2-yl)amino]-3-methoxy-phenyl]piperidin-4-ol;
5-fluoro-4-imidazo[1,2-a]pyridin-3-yl-N-[2-methoxy-5-(4-piperidyl)phenyl]pyrimidin-2-amine;
5-fluoro-4-imidazo[1,2-a]pyridin-3-yl-N-(2-methoxy-4-tetrahydropyran-4-yl-phenyl)pyrimidin-2-amine;
1-[(2R)-2-[[4-[(4-imidazo[1,2-a]pyridin-3-yl-5-methoxy-pyrimidin-2-yl)amino]-3-methoxy-phenoxy]methyl]pyrrolidin-1-yl]ethanone;
2-[4-[4-[(5-bromo-4-imidazo[1,2-a]pyridin-3-yl-pyrimidin-2-yl)amino]-3-methoxy-phenyl]piperazin-1-yl]ethanol;
2-[4-[4-(2-hydroxyethyl)piperazin-1-yl]-2-methoxy-anilino]-4-imidazo[1,2-a]pyridin-3-yl-pyrimidine-5-carbonitrile;
1-[4-[4-[(5-bromo-4-imidazo[1,2-a]pyridin-3-yl-pyrimidin-2-yl)amino]-3-methoxy-phenyl]piperazin-1-yl]ethanone;
2-[4-(4-acetylpiperazin-1-yl)-2-methoxy-anilino]-4-imidazo[1,2-a]pyridin-3-yl-pyrimidine-5-carbonitrile;
5-fluoro-4-imidazo[1,2-a]pyridin-3-yl-N-[2-methoxy-4-(4-methylpiperazin-1-yl)phenyl]pyrimidin-2-amine;
N-[4-(4-ethylpiperazin-1-yl)-2-methoxy-phenyl]-5-fluoro-4-imidazo[1,2-a]pyridin-3-yl-pyrimidin-2-amine;
5-bromo-4-imidazo[1,2-a]pyridin-3-yl-N-[2-methoxy-4-(4-methylpiperazin-1-yl)phenyl]pyrimidin-2-amine;
5-bromo-N-[2-ethoxy-4-(4-methylpiperazin-1-yl)phenyl]-4-imidazo[1,2-a]pyridin-3-yl-pyrimidin-2-amine;
5-bromo-4-imidazo[1,2-a]pyridin-3-yl-N-[2-methoxy-4-(1-methylpyrrolidin-3-yl)oxy-phenyl]pyrimidin-2-amine;
5-bromo-N-(4,5-dimethoxy-2-methyl-phenyl)-4-imidazo[1,2-a]pyridin-3-yl-pyrimidin-2-amine;
1-[4-[4-[[5-chloro-4-[7-(hydroxymethyl)imidazo[1,2-a]pyridin-3-yl]pyrimidin-2-yl]amino]-3-methoxy-phenyl]piperazin-1-yl]ethanone;
1-[4-[4-[[4-(6-bromoimidazo[1,2-a]pyridin-3-yl)-5-chloro-pyrimidin-2-yl]amino]-3-methoxy-phenyl]piperazin-1-yl]ethanone;
3-[2-[4-(4-acetylpiperazin-1-yl)-2-methoxy-anilino]-5-chloro-pyrimidin-4-yl]imidazo[1,2-a]pyridine-6-carbonitrile;
1-[4-[4-[[5-chloro-4-(6,8-difluoroimidazo[1,2-a]pyridin-3-yl)pyrimidin-2-yl]amino]-3-methoxy-phenyl]piperazin-1-yl]ethanone;
1-[4-[4-[[5-chloro-4-(6-methylimidazo[1,2-a]pyridin-3-yl)pyrimidin-2-yl]amino]-3-methoxy-phenyl]piperazin-1-yl]ethanone;
1-[4-[4-[[5-chloro-4-[6-(dimethylamino)imidazo[1,2-a]pyridin-3-yl]pyrimidin-2-yl]amino]-3-methoxy-phenyl]piperazin-1-yl]ethanone;
1-[4-[4-[[5-chloro-4-(6-methoxyimidazo[1,2-a]pyridin-3-yl)pyrimidin-2-yl]amino]-3-methoxy-phenyl]piperazin-1-yl]ethanone;
3-[2-[4-(4-acetylpiperazin-1-yl)-2-methoxy-anilino]-5-chloro-pyrimidin-4-yl]imidazo[1,2-a]pyridine-7-carbonitrile;
1-[4-[4-[[5-chloro-4-(7-ethylimidazo[1,2-a]pyridin-3-yl)pyrimidin-2-yl]amino]-3-methoxy-phenyl]piperazin-1-yl]ethanone;
1-[4-[4-[[5-chloro-4-(7-fluoroimidazo[1,2-a]pyridin-3-yl)pyrimidin-2-yl]amino]-3-methoxy-phenyl]piperazin-1-yl]ethanone;
1-[4-[4-[[5-chloro-4-(7-methylimidazo[1,2-a]pyridin-3-yl)pyrimidin-2-yl]amino]-3-methoxy-phenyl]piperazin-1-yl]ethanone;
1-[4-[4-[[5-chloro-4-(7-methoxyimidazo[1,2-a]pyridin-3-yl)pyrimidin-2-yl]amino]-3-methoxy-phenyl]piperazin-1-yl]ethanone;
1-[4-[4-[[5-chloro-4-[8-(hydroxymethyl)imidazo[1,2-a]pyridin-3-yl]pyrimidin-2-yl]amino]-3-methoxy-phenyl]piperazin-1-yl]ethanone;
3-[2-[4-(4-acetylpiperazin-1-yl)-2-methoxy-anilino]-5-chloro-pyrimidin-4-yl]imidazo[1,2-a]pyridine-8-carbonitrile;
1-[4-[4-[[5-chloro-4-(8-fluoroimidazo[1,2-a]pyridin-3-yl)pyrimidin-2-yl]amino]-3-methoxy-phenyl]piperazin-1-yl]ethanone;
1-[4-[4-[[5-chloro-4-(8-methylimidazo[1,2-a]pyridin-3-yl)pyrimidin-2-yl]amino]-3-methoxy-phenyl]piperazin-1-yl]ethanone;
1-[4-[4-[[5-chloro-4-(8-methoxyimidazo[1,2-a]pyridin-3-yl)pyrimidin-2-yl]amino]-3-methoxy-phenyl]piperazin-1-yl]ethanone;
1-[4-[4-[[4-(8-aminoimidazo[1,2-a]pyridin-3-yl)-5-chloro-pyrimidin-2-yl]amino]-3-methoxy-phenyl]piperazin-1-yl]ethanone;
1-[4-[4-[[5-chloro-4-(7-chloroimidazo[1,2-a]pyridin-3-yl)pyrimidin-2-yl]amino]-3-methoxy-phenyl]piperazin-1-yl]ethanone;
1-[4-[4-[[4-(7-bromoimidazo[1,2-a]pyridin-3-yl)-5-chloro-pyrimidin-2-yl]amino]-3-methoxy-phenyl]piperazin-1-yl]ethanone;
1-[4-[4-[[5-chloro-4-(6-fluoroimidazo[1,2-a]pyridin-3-yl)pyrimidin-2-yl]amino]-3-methoxy-phenyl]piperazin-1-yl]ethanone;
1-[4-[4-[(5-chloro-4-pyrazolo[1,5-a]pyridin-3-yl-pyrimidin-2-yl)amino]-3-methoxy-phenyl]piperazin-1-yl]ethanone;
5-chloro-N-(2-methoxy-4-piperazin-1-yl-phenyl)-4-pyrazolo[1,5-a]pyridin-3-yl-pyrimidin-2-amine;
1-[4-[4-[(5-chloro-4-pyrazolo[1,5-a]pyridin-3-yl-pyrimidin-2-yl)amino]-3-methoxy-phenyl]piperazin-1-yl]-2-hydroxy-ethanone;
(2R)-1-[4-[4-[(5-chloro-4-pyrazolo[1,5-a]pyridin-3-yl-pyrimidin-2-yl)amino]-3-methoxy-phenyl]piperazin-1-yl]-2-hydroxy-propan-1-one;
(2S)-2-amino-1-[4-[4-[(5-chloro-4-pyrazolo[1,5-a]pyridin-3-yl-pyrimidin-2-yl)amino]-3-methoxy-phenyl]piperazin-1-yl]propan-1-one;
1-[4-[4-[(5-chloro-4-pyrazolo[1,5-a]pyridin-3-yl-pyrimidin-2-yl)amino]-3-methoxy-phenyl]piperazin-1-yl]-2-(methylamino)ethanone;
2-[4-[4-[(5-chloro-4-pyrazolo[1,5-a]pyridin-3-yl-pyrimidin-2-yl)amino]-3-methoxy-phenyl]piperazin-1-yl]ethanol;
(2S)-1-[3-[4-[(5-chloro-4-pyrazolo[1,5-a]pyridin-3-yl-pyrimidin-2-yl)amino]-3-methoxy-phenoxy]azetidin-1-yl]-2-hydroxy-propan-1-one;
(2R)-1-[3-[4-[(5-chloro-4-pyrazolo[1,5-a]pyridin-3-yl-pyrimidin-2-yl)amino]-3-methoxy-phenoxy]azetidin-1-yl]-2-hydroxy-propan-1-one;
5-chloro-N-[2-methoxy-4-(4-piperidyl)phenyl]-4-pyrazolo[1,5-a]pyridin-3-yl-pyrimidin-2-amine;
1-[4-[4-[(5-chloro-4-pyrazolo[1,5-a]pyridin-3-yl-pyrimidin-2-yl)amino]-3-methoxy-phenyl]-1-piperidyl]-2-hydroxy-ethanone;
(2R)-1-[4-[4-[(5-chloro-4-pyrazolo[1,5-a]pyridin-3-yl-pyrimidin-2-yl)amino]-3-methoxy-phenyl]-1-piperidyl]-2-hydroxy-propan-1-one;
(2S)-1-[4-[4-[(5-chloro-4-pyrazolo[1,5-a]pyridin-3-yl-pyrimidin-2-yl)amino]-3-methoxy-phenyl]-1-piperidyl]-2-hydroxy-propan-1-one;
1-[4-[4-[(5-chloro-4-imidazo[1,2-a]pyridin-3-yl-pyrimidin-2-yl)amino]-3-methoxy-phenyl]-1-piperidyl]-2-(methylamino)ethanone;
(2R)-2-amino-1-[4-[4-[(5-chloro-4-imidazo[1,2-a]pyridin-3-yl-pyrimidin-2-yl)amino]-3-methoxy-phenyl]-1-piperidyl]propan-1-one;
(2S)-2-amino-1-[4-[4-[(5-chloro-4-imidazo[1,2-a]pyridin-3-yl-pyrimidin-2-yl)amino]-3-methoxy-phenyl]-1-piperidyl]propan-1-one;
5-chloro-4-imidazo[1,2-a]pyridin-3-yl-N-[2-methoxy-4-(9-oxa-3,7-diazabicyclo[3.3.1]nonan-7-yl)phenyl]pyrimidin-2-amine;
2-[7-[4-[(5-chloro-4-imidazo[1,2-a]pyridin-3-yl-pyrimidin-2-yl)amino]-3-methoxy-phenyl]-9-oxa-3,7-diazabicyclo[3.3.1]nonan-3-yl]ethanol;
1-[4-[3-[(5-chloro-4-imidazo[1,2-a]pyridin-3-yl-pyrimidin-2-yl)amino]-4-methoxy-phenyl]piperazin-1-yl]-2-(methylamino)ethanone;
1-[4-[3-[(5-chloro-4-imidazo[1,2-a]pyridin-3-yl-pyrimidin-2-yl)amino]-4-methoxy-phenyl]piperazin-1-yl]-2-(dimethylamino)ethanone;
5-chloro-N-(2-methoxy-5-piperazin-1-yl-phenyl)-4-pyrazolo[1,5-a]pyridin-3-yl-pyrimidin-2-amine;
2-[4-[(5-chloro-4-pyrazolo[1,5-a]pyridin-3-yl-pyrimidin-2-yl)amino]-3-methoxy-phenyl]propane-1,3-diol;
5-chloro-N-[2-methoxy-4-(9-oxa-3,7-diazabicyclo[3.3.1]nonan-7-yl)phenyl]-4-pyrazolo[1,5-a]pyridin-3-yl-pyrimidin-2-amine;
2-[7-[4-[(5-chloro-4-pyrazolo[1,5-a]pyridin-3-yl-pyrimidin-2-yl)amino]-3-methoxy-phenyl]-9-oxa-3,7-diazabicyclo[3.3.1]nonan-3-yl]ethanol;
1-[4-[3-[(5-chloro-4-pyrazolo[1,5-a]pyridin-3-yl-pyrimidin-2-yl)amino]-4-methoxy-phenyl]piperazin-1-yl]-2-(methylamino)ethanone;
1-[4-[3-[(5-chloro-4-pyrazolo[1,5-a]pyridin-3-yl-pyrimidin-2-yl)amino]-4-methoxy-phenyl]piperazin-1-yl]-2-(dimethylamino)ethanone;
N-[3-[(5-chloro-4-pyrazolo[1,5-a]pyridin-3-yl-pyrimidin-2-yl)amino]-4-methoxy-phenyl]-2-(dimethylamino)acetamide;
1-[6-[(5-chloro-4-pyrazolo[1,5-a]pyridin-3-yl-pyrimidin-2-yl)amino]-5-methoxy-indolin-1-yl]ethanone;
N-(5-chloro-4-pyrazolo[1,5-a]pyridin-3-yl-pyrimidin-2-yl)-5-methoxy-indolin-6-amine;
1-[6-[(5-chloro-4-pyrazolo[1,5-a]pyridin-3-yl-pyrimidin-2-yl)amino]-5-methoxy-indolin-1-yl]-2-(methylamino)ethanone;
1-[6-[(5-chloro-4-pyrazolo[1,5-a]pyridin-3-yl-pyrimidin-2-yl)amino]-5-methoxy-indolin-1-yl]-2-(dimethylamino)ethanone;
1-[4-[3-methoxy-4-[(5-methyl-4-pyrazolo[1,5-a]pyridin-3-yl-pyrimidin-2-yl)amino]phenyl]piperazin-1-yl]ethanone;
N-(2-methoxy-4-piperazin-1-yl-phenyl)-5-methyl-4-pyrazolo[1,5-a]pyridin-3-yl-pyrimidin-2-amine;
1-[4-[3-methoxy-4-[(5-methyl-4-pyrazolo[1,5-a]pyridin-3-yl-pyrimidin-2-yl)amino]phenyl]piperazin-1-yl]-2-(methylamino)ethanone;
2-[4-[3-methoxy-4-[(5-methyl-4-pyrazolo[1,5-a]pyridin-3-yl-pyrimidin-2-yl)amino]phenyl]piperazin-1-yl]ethanol;
N-[2-methoxy-4-(4-piperidyl)phenyl]-5-methyl-4-pyrazolo[1,5-a]pyridin-3-yl-pyrimidin-2-amine;
2-hydroxy-1-[4-[3-methoxy-4-[(5-methyl-4-pyrazolo[1,5-a]pyridin-3-yl-pyrimidin-2-yl)amino]phenyl]-1-piperidyl]ethanone;
(2R)-2-hydroxy-1-[4-[3-methoxy-4-[(5-methyl-4-pyrazolo[1,5-a]pyridin-3-yl-pyrimidin-2-yl)amino]phenyl]-1-piperidyl]propan-1-one;
N-(2-methoxy-5-piperazin-1-yl-phenyl)-5-methyl-4-pyrazolo[1,5-a]pyridin-3-yl-pyrimidin-2-amine;
2-[7-[3-methoxy-4-[(5-methyl-4-pyrazolo[1,5-a]pyridin-3-yl-pyrimidin-2-yl)amino]phenyl]-9-oxa-3,7-diazabicyclo[3.3.1]nonan-3-yl]ethanol;
1-[4-[4-methoxy-3-[(5-methyl-4-pyrazolo[1,5-a]pyridin-3-yl-pyrimidin-2-yl)amino]phenyl]piperazin-1-yl]-2-(methylamino)ethanone;
1-[5-methoxy-6-[(5-methyl-4-pyrazolo[1,5-a]pyridin-3-yl-pyrimidin-2-yl)amino]indolin-1-yl]ethanone;
2-(dimethylamino)-1-[5-methoxy-6-[(5-methyl-4-pyrazolo[1,5-a]pyridin-3-yl-pyrimidin-2-yl)amino]indolin-1-yl]ethanone;
1-[4-[4-[(5-fluoro-4-pyrazolo[1,5-a]pyridin-3-yl-pyrimidin-2-yl)amino]-3-methoxy-phenyl]piperazin-1-yl]ethanone;
5-fluoro-N-(2-methoxy-4-piperazin-1-yl-phenyl)-4-pyrazolo[1,5-a]pyridin-3-yl-pyrimidin-2-amine;
1-[4-[4-[(5-fluoro-4-pyrazolo[1,5-a]pyridin-3-yl-pyrimidin-2-yl)amino]-3-methoxy-phenyl]piperazin-1-yl]-2-(methylamino)ethanone;
5-fluoro-N-[2-methoxy-4-(4-piperidyl)phenyl]-4-pyrazolo[1,5-a]pyridin-3-yl-pyrimidin-2-amine;
5-fluoro-N-(2-methoxy-5-piperazin-1-yl-phenyl)-4-pyrazolo[1,5-a]pyridin-3-yl-pyrimidin-2-amine;
2-[7-[4-[(5-fluoro-4-pyrazolo[1,5-a]pyridin-3-yl-pyrimidin-2-yl)amino]-3-methoxy-phenyl]-9-oxa-3,7-diazabicyclo[3.3.1]nonan-3-yl]ethanol;
2-(dimethylamino)-1-[6-[(5-fluoro-4-pyrazolo[1,5-a]pyridin-3-yl-pyrimidin-2-yl)amino]-5-methoxy-indolin-1-yl]ethanone;
1-[4-[4-[[5-chloro-4-(8-fluoroimidazo[1,2-a]pyridin-3-yl)pyrimidin-2-yl]amino]-3-methoxy-phenyl]-1-piperidyl]ethanone;
(2R)-1-[4-[4-[[5-chloro-4-(8-fluoroimidazo[1,2-a]pyridin-3-yl)pyrimidin-2-yl]amino]-3-methoxy-phenyl]piperazin-1-yl]-2-hydroxy-propan-1-one;
1-[4-[4-[[5-chloro-4-(8-fluoroimidazo[1,2-a]pyridin-3-yl)pyrimidin-2-yl]amino]-3-methoxy-phenyl]-1-piperidyl]-2-hydroxy-ethanone;
1-[4-[4-[[5-chloro-4-(8-fluoroimidazo[1,2-a]pyridin-3-yl)pyrimidin-2-yl]amino]-3-methoxy-phenyl]piperazin-1-yl]-2-hydroxy-ethanone;
2-[4-[[5-chloro-4-(8-fluoroimidazo[1,2-a]pyridin-3-yl)pyrimidin-2-yl]amino]-3-methoxy-phenyl]propane-1,3-diol;
1-[4-[4-[[5-chloro-4-(8-fluoroimidazo[1,2-a]pyridin-3-yl)pyrimidin-2-yl]amino]-3-methoxy-phenyl]-1-piperidyl]-2-(methylamino)ethanone;
5-chloro-4-(8-fluoroimidazo[1,2-a]pyridin-3-yl)-N-[2-methoxy-4-(9-oxa-3,7-diazabicyclo[3.3.1]nonan-7-yl)phenyl]pyrimidin-2-amine;
2-[7-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-3-yl]ethanol;
1-[4-[4-[[5-chloro-4-[7-(dimethylaminomethyl)imidazo[1,2-a]pyridin-3-yl]pyrimidin-2-yl]amino]-3-methoxy-phenyl]piperazin-1-yl]ethanone;
1-[4-[4-[[5-chloro-4-[7-(methylaminomethyl)imidazo[1,2-a]pyridin-3-yl]pyrimidin-2-yl]amino]-3-methoxy-phenyl]piperazin-1-yl]ethanone;
1-[4-[4-[[5-chloro-4-[7-(morpholinomethyl)imidazo[1,2-a]pyridin-3-yl]pyrimidin-2-yl]amino]-3-methoxy-phenyl]piperazin-1-yl]ethanone;
N-[[3-[2-[4-(4-acetylpiperazin-1-yl)-2-methoxy-anilino]-5-chloro-pyrimidin-4-yl]imidazo[1,2-a]pyridin-7-yl]methyl]-N-methyl-acetamide;
5-chloro-N-[2-methoxy-5-(9-oxa-3,7-diazabicyclo[3.3.1]nonan-7-yl)phenyl]-4-pyrazolo[1,5-a]pyridin-3-yl-pyrimidin-2-amine;
2-[[4-[(5-chloro-4-pyrazolo[1,5-a]pyridin-3-yl-pyrimidin-2-yl)amino]-3-methoxy-phenyl]methyl]propane-1,3-diol;
N-[3-[[5-chloro-4-(8-fluoroimidazo[1,2-a]pyridin-3-yl)pyrimidin-2-yl]amino]-4-methoxy-phenyl]-2-(dimethylamino)acetamide;
5-chloro-4-(8-fluoroimidazo[1,2-a]pyridin-3-yl)-N-[2-methoxy-5-(9-oxa-3,7-diazabicyclo[3.3.1]nonan-7-yl)phenyl]pyrimidin-2-amine;
2-[[4-[[5-chloro-4-(8-fluoroimidazo[1,2-a]pyridin-3-yl)pyrimidin-2-yl]amino]-3-methoxy-phenyl]methyl]propane-1,3-diol;
2-[4-[4-[[5-chloro-4-(8-fluoroimidazo[1,2-a]pyridin-3-yl)pyrimidin-2-yl]amino]-3-methoxy-phenyl]piperazin-1-yl]ethanol;
2-[4-[4-[[5-fluoro-4-(8-fluoroimidazo[1,2-a]pyridin-3-yl)pyrimidin-2-yl]amino]-3-methoxy-phenyl]piperazin-1-yl]ethanol;
1-[4-[4-[[4-(8-fluoroimidazo[1,2-a]pyridin-3-yl)-5-methyl-pyrimidin-2-yl]amino]-3-methoxy-phenyl]-1-piperidyl]ethanone;
4-(8-fluoroimidazo[1,2-a]pyridin-3-yl)-N-[2-methoxy-4-(4-piperidyl)phenyl]-5-methyl-pyrimidin-2-amine;
1-[4-[4-[[4-(8-fluoroimidazo[1,2-a]pyridin-3-yl)-5-methyl-pyrimidin-2-yl]amino]-3-methoxy-phenyl]-1-piperidyl]-2-hydroxy-ethanone;
4-(8-fluoroimidazo[1,2-a]pyridin-3-yl)-N-(2-methoxy-5-piperazin-1-yl-phenyl)-5-methyl-pyrimidin-2-amine;
(2R)-1-[4-[4-[[4-(8-fluoroimidazo[1,2-a]pyridin-3-yl)-5-methyl-pyrimidin-2-yl]amino]-3-methoxy-phenyl]-1-piperidyl]-2-hydroxy-propan-1-one;
1-[4-[4-[[5-cyclopropyl-4-(8-fluoroimidazo[1,2-a]pyridin-3-yl)pyrimidin-2-yl]amino]-3-methoxy-phenyl]piperazin-1-yl]ethanone;
1-[4-[4-[[5-cyclopropyl-4-(8-fluoroimidazo[1,2-a]pyridin-3-yl)pyrimidin-2-yl]amino]-3-methoxy-phenyl]-1-piperidyl]ethanone;
5-chloro-4-(8-fluoroimidazo[1,2-a]pyridin-3-yl)-N-[2-methoxy-4-(7-methyl-9-oxa-3,7-diazabicyclo[3.3.1]nonan-3-yl)phenyl]pyrimidin-2-amine;
3-[7-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-3-yl]propan-1-ol;
2-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-N,N-dimethyl-acetamide;
3-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-N-methyl-propanamide;
5-chloro-4-(8-fluoroimidazo[1,2-a]pyridin-3-yl)-N-[2-methoxy-4-[3-(2-methylsulfonylethyl)-9-oxa-3,7-diazabicyclo[3.3.1]nonan-7-yl]phenyl]pyrimidin-2-amine;
1-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-2-(methylamino)ethanone;
2-amino-1-[7-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-3-yl]ethanone;
(2S)-2-amino-1-[7-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-3-yl]-3-hydroxy-propan-1-one;
1-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-2-(dimethylamino)ethanone;
1-[7-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-3-yl]-2-hydroxy-ethanone;
(2S)-1-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-2-hydroxy-propan-1-one;
(2R)-1-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-2-hydroxy-propan-1-one;
1-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]ethanone;
2-[4-[4-[[5-chloro-4-(8-fluoroimidazo[1,2-a]pyridin-3-yl)pyrimidin-2-yl]amino]-3-methoxy-phenyl]-1-piperidyl]-N,N-dimethyl-acetamide;
2-[4-[[5-chloro-4-(8-fluoroimidazo[1,2-a]pyridin-3-yl)pyrimidin-2-yl]amino]-3-methoxy-phenoxy]-N,N-dimethyl-acetamide;
(2S)-2-[4-[[5-chloro-4-(8-fluoroimidazo[1,2-a]pyridin-3-yl)pyrimidin-2-yl]amino]-3-methoxy-phenoxy]-N-methyl-propanamide;
(2R)-2-[4-[[5-chloro-4-(8-fluoroimidazo[1,2-a]pyridin-3-yl)pyrimidin-2-yl]amino]-3-methoxy-phenoxy]-N-methyl-propanamide;
N-[2-[4-[[5-chloro-4-(8-fluoroimidazo[1,2-a]pyridin-3-yl)pyrimidin-2-yl]amino]-3-methoxy-N-methyl-anilino]ethyl]-2-hydroxy-N-methyl-acetamide;
2-[2-[4-[[5-chloro-4-(8-fluoroimidazo[1,2-a]pyridin-3-yl)pyrimidin-2-yl]amino]-3-methoxy-N-methyl-anilino]ethyl-methyl-amino]ethanol;
1-[4-[[5-chloro-4-(8-fluoroimidazo[1,2-a]pyridin-3-yl)pyrimidin-2-yl]amino]-3-methoxy-phenyl]-N-methyl-piperidine-4-carboxamide;
2-[4-[[5-chloro-4-(8-fluoroimidazo[1,2-a]pyridin-3-yl)pyrimidin-2-yl]amino]-N-(2-hydroxyethyl)-3-methoxy-anilino]ethanol;
1-[4-[[5-chloro-4-(8-fluoroimidazo[1,2-a]pyridin-3-yl)pyrimidin-2-yl]amino]-3-methoxy-phenyl]piperazin-2-one;
2-[4-[[5-chloro-4-(8-fluoroimidazo[1,2-a]pyridin-3-yl)pyrimidin-2-yl]amino]-3-methoxy-N-methyl-anilino]-N,N-dimethyl-acetamide;
2-[4-[[5-chloro-4-(8-fluoroimidazo[1,2-a]pyridin-3-yl)pyrimidin-2-yl]amino]-3-methoxy-N-methyl-anilino]-N-methyl-acetamide;
2-[4-[[5-chloro-4-(8-fluoroimidazo[1,2-a]pyridin-3-yl)pyrimidin-2-yl]amino]-3-methoxy-N-methyl-anilino]-1-piperazin-1-yl-ethanone;
2-[4-[4-[(5-fluoro-4-imidazo[1,2-a]pyridin-3-yl-pyrimidin-2-yl)amino]-3-methoxy-phenyl]piperazin-1-yl]-2-methyl-propan-1-ol;
2-[4-[4-[(4-imidazo[1,2-a]pyridin-3-yl-5-methyl-pyrimidin-2-yl)amino]-3-methoxy-phenyl]piperazin-1-yl]-2-methyl-propan-1-ol;
2-[4-[4-[(5-chloro-4-pyrazolo[1,5-a]pyridin-3-yl-pyrimidin-2-yl)amino]-3-methoxy-phenyl]piperazin-1-yl]-2-methyl-propan-1-ol;
2-[4-[3-methoxy-4-[(5-methyl-4-pyrazolo[1,5-a]pyridin-3-yl-pyrimidin-2-yl)amino]phenyl]piperazin-1-yl]-2-methyl-propan-1-ol;
2-[7-[4-[(4-imidazo[1,2-a]pyridin-3-yl-5-methyl-pyrimidin-2-yl)amino]-3-methoxy-phenyl]-9-oxa-3,7-diazabicyclo[3.3.1]nonan-3-yl]ethanol;
1-[4-[3-methoxy-4-[(5-methyl-4-pyrazolo[1,5-a]pyridin-3-yl-pyrimidin-2-yl)amino]phenyl]-1-piperidyl]ethanone;
1-[4-[4-[(5-cyclopropyl-4-pyrazolo[1,5-a]pyridin-3-yl-pyrimidin-2-yl)amino]-3-methoxy-phenyl]piperazin-1-yl]ethanone;
(2R)-2-amino-1-[4-[3-methoxy-4-[(5-methyl-4-pyrazolo[1,5-a]pyridin-3-yl-pyrimidin-2-yl)amino]phenyl]piperazin-1-yl]propan-1-one;
2-(dimethylamino)-1-[4-[4-methoxy-3-[(5-methyl-4-pyrazolo[1,5-a]pyridin-3-yl-pyrimidin-2-yl)amino]phenyl]piperazin-1-yl]ethanone;
1-[4-[3-methoxy-4-[(5-methyl-4-pyrazolo[1,5-a]pyridin-3-yl-pyrimidin-2-yl)amino]phenyl]-1-piperidyl]-2-(methylamino)ethanone;
1-[4-[4-[(5-fluoro-4-pyrazolo[1,5-a]pyridin-3-yl-pyrimidin-2-yl)amino]-3-methoxy-phenyl]-1-piperidyl]-2-(methylamino)ethanone;
2-[4-(1-acetyl-4-piperidyl)-2-methoxy-anilino]-4-pyrazolo[1,5-a]pyridin-3-yl-pyrimidine-5-carbonitrile;
2-[4-[1-(2-hydroxyacetyl)-4-piperidyl]-2-methoxy-anilino]-4-pyrazolo[1,5-a]pyridin-3-yl-pyrimidine-5-carbonitrile;
1-[(1S,4S)-3-[4-[(5-chloro-4-pyrazolo[1,5-a]pyridin-3-yl-pyrimidin-2-yl)amino]-3-methoxy-phenyl]-3,6-diazabicyclo[2.2.1]heptan-6-yl]ethanone;
2-[7-[4-[(5-chloro-4-pyrazolo[1,5-a]pyridin-3-yl-pyrimidin-2-yl)amino]-3-methoxy-phenyl]-9-oxa-3,7-diazabicyclo[3.3.1]nonan-3-yl]propane-1,3-diol;
1-[4-[4-[[5-chloro-4-(7-pyrrolidin-1-ylimidazo[1,2-a]pyridin-3-yl)pyrimidin-2-yl]amino]-3-methoxy-phenyl]piperazin-1-yl]ethanone;
1-[4-[4-[[5-chloro-4-(6-fluoroimidazo[1,2-a]pyridin-3-yl)pyrimidin-2-yl]amino]-3-methoxy-phenyl]-1-piperidyl]-2-hydroxy-ethanone;
1-[4-[4-[[5-chloro-4-(7-methylimidazo[1,2-a]pyridin-3-yl)pyrimidin-2-yl]amino]-3-methoxy-phenyl]-1-piperidyl]-2-hydroxy-ethanone;
(2R)-1-[4-[4-[[5-chloro-4-(6-fluoroimidazo[1,2-a]pyridin-3-yl)pyrimidin-2-yl]amino]-3-methoxy-phenyl]-1-piperidyl]-2-hydroxy-propan-1-one;
5-chloro-4-(6-fluoroimidazo[1,2-a]pyridin-3-yl)-N-[2-methoxy-4-(4-piperidyl)phenyl]pyrimidin-2-amine;
1-[4-[4-[[5-chloro-4-(6,8-difluoroimidazo[1,2-a]pyridin-3-yl)pyrimidin-2-yl]amino]-3-methoxy-phenyl]piperazin-1-yl]-2-(methylamino)ethanone;
1-[4-[4-[[5-chloro-4-(6-fluoroimidazo[1,2-a]pyridin-3-yl)pyrimidin-2-yl]amino]-3-methoxy-phenyl]piperazin-1-yl]-2-hydroxy-ethanone;
1-[4-[4-[[5-chloro-4-(6,8-difluoroimidazo[1,2-a]pyridin-3-yl)pyrimidin-2-yl]amino]-3-methoxy-phenyl]piperazin-1-yl]-2-hydroxy-ethanone;
1-[4-[4-[[5-chloro-4-(7-methylimidazo[1,2-a]pyridin-3-yl)pyrimidin-2-yl]amino]-3-methoxy-phenyl]piperazin-1-yl]-2-hydroxy-ethanone;
(2R)-2-amino-1-[4-[4-[[5-chloro-4-(6-fluoroimidazo[1,2-a]pyridin-3-yl)pyrimidin-2-yl]amino]-3-methoxy-phenyl]piperazin-1-yl]propan-1-one;
(2R)-2-amino-1-[4-[4-[[5-chloro-4-(6,8-difluoroimidazo[1,2-a]pyridin-3-yl)pyrimidin-2-yl]amino]-3-methoxy-phenyl]piperazin-1-yl]propan-1-one;
(2R)-2-amino-1-[4-[4-[[5-chloro-4-(7-methylimidazo[1,2-a]pyridin-3-yl)pyrimidin-2-yl]amino]-3-methoxy-phenyl]piperazin-1-yl]propan-1-one;
(2R)-1-[4-[4-[[5-chloro-4-(6-fluoroimidazo[1,2-a]pyridin-3-yl)pyrimidin-2-yl]amino]-3-methoxy-phenyl]piperazin-1-yl]-2-hydroxy-propan-1-one;
(2R)-1-[4-[4-[[5-chloro-4-(6,8-difluoroimidazo[1,2-a]pyridin-3-yl)pyrimidin-2-yl]amino]-3-methoxy-phenyl]piperazin-1-yl]-2-hydroxy-propan-1-one;
(2R)-1-[4-[4-[[5-chloro-4-(7-methylimidazo[1,2-a]pyridin-3-yl)pyrimidin-2-yl]amino]-3-methoxy-phenyl]piperazin-1-yl]-2-hydroxy-propan-1-one;
(2S)-2-amino-1-[4-[4-[[5-chloro-4-(7-methylimidazo[1,2-a]pyridin-3-yl)pyrimidin-2-yl]amino]-3-methoxy-phenyl]piperazin-1-yl]propan-1-one;
2-[4-[4-[[5-chloro-4-(6-fluoroimidazo[1,2-a]pyridin-3-yl)pyrimidin-2-yl]amino]-3-methoxy-phenyl]piperazin-1-yl]-2-methyl-propan-1-ol;
2-[4-[4-[[4-(6-fluoroimidazo[1,2-a]pyridin-3-yl)-5-methyl-pyrimidin-2-yl]amino]-3-methoxy-phenyl]piperazin-1-yl]-2-methyl-propan-1-ol;
2-[4-[4-[[5-fluoro-4-(6-fluoroimidazo[1,2-a]pyridin-3-yl)pyrimidin-2-yl]amino]-3-methoxy-phenyl]piperazin-1-yl]-2-methyl-propan-1-ol;
2-[4-[3-methoxy-4-[[5-methyl-4-(7-methylimidazo[1,2-a]pyridin-3-yl)pyrimidin-2-yl]amino]phenyl]piperazin-1-yl]-2-methyl-propan-1-ol;
2-[4-[4-[[5-chloro-4-(6-fluoroimidazo[1,2-a]pyridin-3-yl)pyrimidin-2-yl]amino]-3-methoxy-phenyl]piperazin-1-yl]ethanol;
2-[4-[4-[[4-(6-fluoroimidazo[1,2-a]pyridin-3-yl)-5-methyl-pyrimidin-2-yl]amino]-3-methoxy-phenyl]piperazin-1-yl]ethanol;
5-chloro-4-(6,8-difluoroimidazo[1,2-a]pyridin-3-yl)-N-(2-methoxy-4-piperazin-1-yl-phenyl)pyrimidin-2-amine;
3-[5-chloro-2-(2-methoxy-4-piperazin-1-yl-anilino)pyrimidin-4-yl]imidazo[1,2-a]pyridine-6-carbonitrile;
(2S)-3-[4-[4-[(4-imidazo[1,2-a]pyridin-3-yl-5-methyl-pyrimidin-2-yl)amino]-3-methoxy-phenyl]piperazin-1-yl]propane-1,2-diol;
(2R)-3-[4-[4-[(4-imidazo[1,2-a]pyridin-3-yl-5-methyl-pyrimidin-2-yl)amino]-3-methoxy-phenyl]piperazin-1-yl]propane-1,2-diol;
2-[4-[4-[(4-imidazo[1,2-a]pyridin-3-yl-5-methyl-pyrimidin-2-yl)amino]-3-methoxy-phenyl]piperazin-1-yl]propane-1,3-diol;
2-[(2R)-4-[4-[(4-imidazo[1,2-a]pyridin-3-yl-5-methyl-pyrimidin-2-yl)amino]-3-methoxy-phenyl]-2-methyl-piperazin-1-yl]ethanol;
2-[(2S)-4-[4-[(4-imidazo[1,2-a]pyridin-3-yl-5-methyl-pyrimidin-2-yl)amino]-3-methoxy-phenyl]-2-methyl-piperazin-1-yl]ethanol;
2-[(2R)-4-[4-[(5-fluoro-4-imidazo[1,2-a]pyridin-3-yl-pyrimidin-2-yl)amino]-3-methoxy-phenyl]-2-methyl-piperazin-1-yl]ethanol;
2-[(2S)-4-[4-[(5-fluoro-4-imidazo[1,2-a]pyridin-3-yl-pyrimidin-2-yl)amino]-3-methoxy-phenyl]-2-methyl-piperazin-1-yl]ethanol;
2-[4-[4-[(4-imidazo[1,2-a]pyridin-3-yl-5-methyl-pyrimidin-2-yl)amino]-3-methoxy-phenyl]piperazin-1-yl]propan-1-ol;
1-[4-[4-[(5-chloro-4-imidazo[1,2-a]pyridin-3-yl-pyrimidin-2-yl)amino]-3-methoxy-phenyl]piperazin-1-yl]-2-morpholino-ethanone;
N-[2-[4-[(5-chloro-4-imidazo[1,2-a]pyridin-3-yl-pyrimidin-2-yl)amino]-3-methoxy-phenyl]-3-hydroxy-propyl]acetamide;
1-[3-[4-[(5-chloro-4-imidazo[1,2-a]pyridin-3-yl-pyrimidin-2-yl)amino]-3-methoxy-phenyl]azetidin-1-yl]ethanone;
1-[4-[4-[(5-chloro-4-imidazo[1,2-a]pyridin-3-yl-pyrimidin-2-yl)amino]-5-methoxy-2-methyl-phenyl]piperazin-1-yl]ethanone;
2-[4-[4-[(5-chloro-4-imidazo[1,2-a]pyridin-3-yl-pyrimidin-2-yl)amino]-5-methoxy-2-methyl-phenyl]piperazin-1-yl]ethanol;
1-[4-[4-[(5-chloro-4-imidazo[1,2-a]pyridin-3-yl-pyrimidin-2-yl)amino]-3-methoxy-2-methyl-phenyl]piperazin-1-yl]ethanone;
1-[4-[4-[(5-chloro-4-imidazo[1,2-a]pyridin-3-yl-pyrimidin-2-yl)amino]-2,3-dimethoxy-phenyl]piperazin-1-yl]ethanone;
1-[4-[4-[(5-chloro-4-imidazo[1,2-a]pyridin-3-yl-pyrimidin-2-yl)amino]-2-fluoro-3-methoxy-phenyl]piperazin-1-yl]ethanone;
6-(4-acetylpiperazin-1-yl)-3-[(5-chloro-4-imidazo[1,2-a]pyridin-3-yl-pyrimidin-2-yl)amino]-2-methoxy-benzonitrile;
1-[4-[4-[(5-chloro-4-imidazo[1,2-a]pyridin-3-yl-pyrimidin-2-yl)amino]-2,5-dimethoxy-phenyl]piperazin-1-yl]ethanone;
2-(4-acetylpiperazin-1-yl)-5-[(5-chloro-4-imidazo[1,2-a]pyridin-3-yl-pyrimidin-2-yl)amino]-4-methoxy-benzonitrile;
1-[4-[4-[(5-chloro-4-imidazo[1,2-a]pyridin-3-yl-pyrimidin-2-yl)amino]-3-(2-methoxyethoxy)phenyl]piperazin-1-yl]ethanone;
5-(4-acetylpiperazin-1-yl)-2-[(5-chloro-4-imidazo[1,2-a]pyridin-3-yl-pyrimidin-2-yl)amino]benzonitrile;
1-[4-[4-[(5-chloro-4-imidazo[1,2-a]pyridin-3-yl-pyrimidin-2-yl)amino]-3-(hydroxymethyl)phenyl]piperazin-1-yl]ethanone;
1-[4-[4-[(5-chloro-4-imidazo[1,2-a]pyridin-3-yl-pyrimidin-2-yl)amino]-3-(methoxymethyl)phenyl]piperazin-1-yl]ethanone;
5-chloro-4-imidazo[1,2-a]pyridin-3-yl-N-[2-methoxy-4-(3-oxa-7,9-diazabicyclo[3.3.1]nonan-7-yl)phenyl]pyrimidin-2-amine;
1-[3-[4-[(5-chloro-4-imidazo[1,2-a]pyridin-3-yl-pyrimidin-2-yl)amino]-3-methoxy-phenyl]-7-oxa-3,9-diazabicyclo[3.3.1]nonan-9-yl]ethanone
   and pharmaceutically acceptable salts thereof.

Particular compounds of the invention include, for example, any one or more compounds of formula (I) selected from:
1-[4-[4-[(5-chloro-4-imidazo[1,2-a]pyridin-3-yl-pyrimidin-2-yl)amino]-3-methoxy-phenyl]piperazin-1-yl]ethanone;
5-chloro-4-imidazo[1,2-a]pyridin-3-yl-N-(2-methoxy-4-piperazin-1-yl-phenyl)pyrimidin-2-amine;
[4-[(5-chloro-4-imidazo[1,2-a]pyridin-3-yl-pyrimidin-2-yl)amino]-3-methoxy-phenyl]-morpholino-methanone;
[1-[4-[(5-chloro-4-imidazo[1,2-a]pyridin-3-yl-pyrimidin-2-yl)amino]-3-methoxy-phenyl]pyrrolidin-2-yl]-morpholino-methanone;
2-[4-[4-[(5-chloro-4-imidazo[1,2-a]pyridin-3-yl-pyrimidin-2-yl)amino]-3-methoxy-phenyl]piperazin-1-yl]-1-morpholino-ethanone;
[3-[(5-chloro-4-imidazo[1,2-a]pyridin-3-yl-pyrimidin-2-yl)amino]-4-methoxy-phenyl]-morpholino-methanone;
(3S)-1-[3-[(5-chloro-4-imidazo[1,2-a]pyridin-3-yl-pyrimidin-2-yl)amino]-4-methoxy-phenyl]pyrrolidin-3-ol;
5-chloro-N-[4-(3,5-dimethylpiperazin-1-yl)-2-methoxy-phenyl]-4-imidazo[1,2-a]pyridin-3-yl-pyrimidin-2-amine;
4-[4-[(5-chloro-4-imidazo[1,2-a]pyridin-3-yl-pyrimidin-2-yl)amino]-3-methoxy-phenyl]-N,N-dimethyl-piperazine-1-sulfonamide;
5-chloro-4-imidazo[1,2-a]pyridin-3-yl-N-(2-methoxy-5-pyrrolidin-1-yl-phenyl)pyrimidin-2-amine;
1-[4-[3-[(5-chloro-4-imidazo[1,2-a]pyridin-3-yl-pyrimidin-2-yl)amino]-4-methoxy-phenyl]piperazin-1-yl]ethanone;
5-chloro-4-imidazo[1,2-a]pyridin-3-yl-N-(2-methoxy-5-piperazin-1-yl-phenyl)pyrimidin-2-amine;
5-chloro-N-[4-[(3R)-3-dimethylaminopyrrolidin-1-yl]-2-methoxy-phenyl]-4-imidazo[1,2-a]pyridin-3-yl-pyrimidin-2-amine;
5-chloro-4-imidazo[1,2-a]pyridin-3-yl-N-[2-methoxy-4-[2-[4-(2-methoxyethyl)piperazin-1-yl]ethoxy]phenyl]pyrimidin-2-amine;
5-chloro-N-[5-[(3R)-3-dimethylaminopyrrolidin-1-yl]-2-methoxy-phenyl]-4-imidazo[1,2-a]pyridin-3-yl-pyrimidin-2-amine;
5-chloro-N-[5-[(3S)-3-dimethylaminopyrrolidin-1-yl]-2-methyl-phenyl]-4-imidazo[1,2-a]pyridin-3-yl-pyrimidin-2-amine;
5-chloro-N-[4-(1,1-dioxo-1,4-thiazinan-4-yl)-2-methoxy-phenyl]-4-imidazo[1,2-a]pyridin-3-yl-pyrimidin-2-amine;
5-chloro-N-[4-[(3S)-3-dimethylaminopyrrolidin-1-yl]-2-methoxy-phenyl]-4-imidazo[1,2-a]pyridin-3-yl-pyrimidin-2-amine;
5-chloro-N-[5-[(3S)-3-dimethylaminopyrrolidin-1-yl]-2-methoxy-phenyl]-4-imidazo[1,2-a]pyridin-3-yl-pyrimidin-2-amine;
(3R)-1-[3-[(5-chloro-4-imidazo[1,2-a]pyridin-3-yl-pyrimidin-2-yl)amino]-4-methoxy-phenyl]pyrrolidin-3-ol;
5-chloro-4-imidazo[1,2-a]pyridin-3-yl-N-(2-methoxy-5-morpholino-phenyl)pyrimidin-2-amine;
5-chloro-4-imidazo[1,2-a]pyridin-3-yl-N-[2-methoxy-5-(4-methoxy-1-piperidyl)phenyl]pyrimidin-2-amine;
1-[4-[4-[(5-chloro-4-imidazo[1,2-a]pyridin-3-yl-pyrimidin-2-yl)amino]-3-methoxy-phenyl]-1-piperidyl]ethanone;
5-chloro-4-imidazo[1,2-a]pyridin-3-yl-N-[2-methoxy-4-(4-methylsulfonylpiperazin-1-yl)phenyl]pyrimidin-2-amine;
N-[(3R)-1-[4-[(5-chloro-4-imidazo[1,2-a]pyridin-3-yl-pyrimidin-2-yl)amino]-3-methoxy-phenyl]pyrrolidin-3-yl]acetamide;
4-[4-[(5-chloro-4-imidazo[1,2-a]pyridin-3-yl-pyrimidin-2-yl)amino]-3-methoxy-phenyl]pyrrolidin-2-one;
1-[2-[4-[(5-chloro-4-imidazo[1,2-a]pyridin-3-yl-pyrimidin-2-yl)amino]-3-methoxy-phenyl]-1-piperidyl]ethanone;
1-[4-[4-[(5-chloro-4-imidazo[1,2-a]pyridin-3-yl-pyrimidin-2-yl)amino]-3-fluoro-5-methoxy-phenyl]piperazin-1-yl]ethanone;
4-[4-[(5-chloro-4-imidazo[1,2-a]pyridin-3-yl-pyrimidin-2-yl)amino]-3-methoxy-phenyl]-N-methyl-pyrrolidine-2-carboxamide;
1-[4-[4-[(5-chloro-4-imidazo[1,2-a]pyridin-3-yl-pyrimidin-2-yl)amino]-3-methoxy-phenyl]-1,4-diazepan-1-yl]ethanone;
1-[3-[4-[(5-chloro-4-imidazo[1,2-a]pyridin-3-yl-pyrimidin-2-yl)amino]-3-methoxy-phenyl]-1-piperidyl]ethanone;
1-[4-[4-[(5-chloro-4-imidazo[1,2-a]pyridin-3-yl-pyrimidin-2-yl)amino]-3-methyl-phenyl]piperazin-1-yl]ethanone;
1-[4-[4-[(5-chloro-4-imidazo[1,2-a]pyridin-3-yl-pyrimidin-2-yl)amino]-3-methoxy-phenyl]-2,6-dimethyl-piperazin-1-yl]ethanone;
1-[4-[4-[(5-chloro-4-imidazo[1,2-a]pyridin-3-yl-pyrimidin-2-yl)amino]-2,3-dimethyl-phenyl]piperazin-1-yl]ethanone;
1-[4-[4-[(5-chloro-4-imidazo[1,2-a]pyridin-3-yl-pyrimidin-2-yl)amino]-2-fluoro-5-methyl-phenyl]piperazin-1-yl]ethanone;
1-[4-[4-[(5-chloro-4-imidazo[1,2-a]pyridin-3-yl-pyrimidin-2-yl)amino]-3-isopropoxy-phenyl]piperazin-1-yl]ethanone;
1-[(2R,5S)-4-[4-[(5-chloro-4-imidazo[1,2-a]pyridin-3-yl-pyrimidin-2-yl)amino]-3-methoxy-phenyl]-2,5-dimethyl-piperazin-1-yl]ethanone;
1-[4-[4-[(5-chloro-4-imidazo[1,2-a]pyridin-3-yl-pyrimidin-2-yl)amino]-3-methoxy-phenyl]-2-methyl-piperazin-1-yl]ethanone;
1-[4-[3-[(5-chloro-4-imidazo[1,2-a]pyridin-3-yl-pyrimidin-2-yl)amino]-2-methyl-phenyl]piperazin-1-yl]ethanone;
1-[4-[4-[(5-chloro-4-imidazo[1,2-a]pyridin-3-yl-pyrimidin-2-yl)amino]-3-methoxy-phenyl]-2-(hydroxymethyl)piperazin-1-yl]ethanone;
2-[4-[(5-chloro-4-imidazo[1,2-a]pyridin-3-yl-pyrimidin-2-yl)amino]-3-methoxy-phenyl]-1-morpholino-propan-1-one;
1-[(6S)-4-[4-[(5-chloro-4-imidazo[1,2-a]pyridin-3-yl-pyrimidin-2-yl)amino]-3-methoxy-phenyl]-6-hydroxy-1,4-diazepan-1-yl]ethanone;
2-[4-[(5-chloro-4-imidazo[1,2-a]pyridin-3-yl-pyrimidin-2-yl)amino]-3-methoxy-phenyl]-1-[4-(2-hydroxyethyl)piperazin-1-yl]propan-1-one;
2-[4-[(5-chloro-4-imidazo[1,2-a]pyridin-3-yl-pyrimidin-2-yl)amino]-3-methoxy-phenyl]-1-piperazin-1-yl-propan-1-one;
5-chloro-4-imidazo[1,2-a]pyridin-3-yl-N-(2-methoxy-4-morpholino-phenyl)pyrimidin-2-amine;
5-chloro-4-imidazo[1,2-a]pyridin-3-yl-N-(2-methoxy-4-pyrrolidin-1-yl-phenyl)pyrimidin-2-amine;
2-[4-[4-[(5-chloro-4-imidazo[1,2-a]pyridin-3-yl-pyrimidin-2-yl)amino]-3-methoxy-phenyl]piperazin-1-yl]ethanol;
5-chloro-4-imidazo[1,2-a]pyridin-3-yl-N-[2-methoxy-4-[4-(2-methoxyethyl)piperazin-1-yl]phenyl]pyrimidin-2-amine;
(3S)-1-[4-[(5-chloro-4-imidazo[1,2-a]pyridin-3-yl-pyrimidin-2-yl)amino]-3-methoxy-phenyl]pyrrolidin-3-ol;
5-chloro-4-imidazo[1,2-a]pyridin-3-yl-N-[2-methoxy-4-(1,4-oxazepan-4-yl)phenyl]pyrimidin-2-amine;
(3R)-1-[4-[(5-chloro-4-imidazo[1,2-a]pyridin-3-yl-pyrimidin-2-yl)amino]-3-methoxy-phenyl]pyrrolidin-3-ol;
5-chloro-4-imidazo[1,2-a]pyridin-3-yl-N-[2-methoxy-4-(4-piperidyl)phenyl]pyrimidin-2-amine;
5-chloro-4-imidazo[1,2-a]pyridin-3-yl-N-[2-methoxy-4-(4-piperidyloxy)phenyl]pyrimidin-2-amine;
5-chloro-4-imidazo[1,2-a]pyridin-3-yl-N-[2-methoxy-4-[[(2R)-pyrrolidin-2-yl]methoxy]phenyl]pyrimidin-2-amine;
5-chloro-4-imidazo[1,2-a]pyridin-3-yl-N-[2-methoxy-4-[[(2S)-pyrrolidin-2-yl]methoxy]phenyl]pyrimidin-2-amine;
5-chloro-4-imidazo[1,2-a]pyridin-3-yl-N-[2-methoxy-5-[[(2S)-pyrrolidin-2-yl]methoxy]phenyl]pyrimidin-2-amine;
5-chloro-4-imidazo[1,2-a]pyridin-3-yl-N-[2-methoxy-5-[[(2R)-pyrrolidin-2-yl]methoxy]phenyl]pyrimidin-2-amine;
5-chloro-4-imidazo[1,2-a]pyridin-3-yl-N-[2-methoxy-4-[(3R)-pyrrolidin-3-yl]oxy-phenyl]pyrimidin-2-amine;
5-chloro-4-imidazo[1,2-a]pyridin-3-yl-N-[2-methoxy-4-[(3S)-pyrrolidin-3-yl]oxy-phenyl]pyrimidin-2-amine;
1-[4-[4-[(5-chloro-4-imidazo[1,2-a]pyridin-3-yl-pyrimidin-2-yl)amino]-3-methoxy-phenoxy]-1-piperidyl]ethanone;
5-chloro-4-imidazo[1,2-a]pyridin-3-yl-N-[2-methoxy-4-[(1-methylsulfonyl-4-piperidyl)oxy]phenyl]pyrimidin-2-amine;
1-[4-[4-[(5-chloro-4-imidazo[1,2-a]pyridin-3-yl-pyrimidin-2-yl)amino]-3-methoxy-phenoxy]-1-piperidyl]-2-hydroxy-propan-1-one;
1-[(3R)-3-[4-[(5-chloro-4-imidazo[1,2-a]pyridin-3-yl-pyrimidin-2-yl)amino]-3-methoxy-phenoxy]pyrrolidin-1-yl]ethanone;
5-chloro-4-imidazo[1,2-a]pyridin-3-yl-N-[2-methoxy-4-[(3R)-1-methylsulfonylpyrrolidin-3-yl]oxy-phenyl]pyrimidin-2-amine;
1-[(3R)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one;
(S)-2-hydroxypropanoic acid, (2S)-1-[(3R)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one;
1-[(3S)-3-[4-[(5-chloro-4-imidazo[1,2-a]pyridin-3-yl-pyrimidin-2-yl)amino]-3-methoxy-phenoxy]pyrrolidin-1-yl]ethanone;
5-chloro-4-imidazo[1,2-a]pyridin-3-yl-N-[2-methoxy-4-[(3S)-1-methylsulfonylpyrrolidin-3-yl]oxy-phenyl]pyrimidin-2-amine;
1-[(3S)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one;
1-[(2S)-2-[[4-[(5-chloro-4-imidazo[1,2-a]pyridin-3-yl-pyrimidin-2-yl)amino]-3-methoxy-phenoxy]methyl]pyrrolidin-1-yl]ethanone;
1-[(2R)-2-[[3-[(5-chloro-4-imidazo[1,2-a]pyridin-3-yl-pyrimidin-2-yl)amino]-4-methoxy-phenoxy]methyl]pyrrolidin-1-yl]ethanone;
4-[4-[(5-chloro-4-imidazo[1,2-a]pyridin-3-yl-pyrimidin-2-yl)amino]-3-methoxy-phenyl]piperazin-2-one;
4-[4-[(5-chloro-4-imidazo[1,2-a]pyridin-3-yl-pyrimidin-2-yl)amino]-3-methoxy-phenyl]-N,N-dimethyl-piperazine-2-carboxamide;
(3S)-1-[[4-[(5-chloro-4-imidazo[1,2-a]pyridin-3-yl-pyrimidin-2-yl)amino]-3-methoxy-phenyl]methyl]pyrrolidin-3-ol;
2-[[4-[(5-chloro-4-imidazo[1,2-a]pyridin-3-yl-pyrimidin-2-yl)amino]-3-methoxy-phenyl]methylamino]ethanol;
2-[[4-[(5-chloro-4-imidazo[1,2-a]pyridin-3-yl-pyrimidin-2-yl)amino]-3-methoxy-phenyl]methyl-methyl-amino]ethanol;
3-[[4-[(5-chloro-4-imidazo[1,2-a]pyridin-3-yl-pyrimidin-2-yl)amino]-3-methoxy-phenyl]methylamino]propan-1-ol;
5-chloro-4-imidazo[1,2-a]pyridin-3-yl-N-[2-methoxy-4-[(4-methylpiperazin-1-yl)methyl]phenyl]pyrimidin-2-amine;
1-[[4-[(5-chloro-4-imidazo[1,2-a]pyridin-3-yl-pyrimidin-2-yl)amino]-3-methoxy-phenyl]methyl]piperidin-4-ol;
1-[4-[[4-[(5-chloro-4-imidazo[1,2-a]pyridin-3-yl-pyrimidin-2-yl)amino]-3-methoxy-phenyl]methyl]piperazin-1-yl]ethanone;
1-[[4-[(5-chloro-4-imidazo[1,2-a]pyridin-3-yl-pyrimidin-2-yl)amino]-3-methoxy-phenyl]methyl]piperidine-4-carboxamide;
2-[4-[[4-[(5-chloro-4-imidazo[1,2-a]pyridin-3-yl-pyrimidin-2-yl)amino]-3-methoxy-phenyl]methyl]piperazin-1-yl]ethanol;
5-chloro-4-imidazo[1,2-a]pyridin-3-yl-N-[2-methoxy-4-[(3-methoxypropylamino)methyl]phenyl]pyrimidin-2-amine;
N-[4-(azetidin-1-ylmethyl)-2-methoxy-phenyl]-5-chloro-4-imidazo[1,2-a]pyridin-3-yl-pyrimidin-2-amine;
5-chloro-4-imidazo[1,2-a]pyridin-3-yl-N-[2-methoxy-4-[(4-methyl-1,4-diazepan-1-yl)methyl]phenyl]pyrimidin-2-amine;
(3R)-1-[[4-[(5-chloro-4-imidazo[1,2-a]pyridin-3-yl-pyrimidin-2-yl)amino]-3-methoxy-phenyl]methyl]piperidin-3-ol;
5-chloro-4-imidazo[1,2-a]pyridin-3-yl-N-[2-methoxy-4-[(2-methoxyethyl-methyl-amino)methyl]phenyl]pyrimidin-2-amine;
5-chloro-4-imidazo[1,2-a]pyridin-3-yl-N-[2-methoxy-4-(1,4-oxazepan-4-ylmethyl)phenyl]pyrimidin-2-amine;
(2R)-2-[[4-[(5-chloro-4-imidazo[1,2-a]pyridin-3-yl-pyrimidin-2-yl)amino]-3-methoxy-phenyl]methylamino]propan-1-ol;
1-[[4-[(5-chloro-4-imidazo[1,2-a]pyridin-3-yl-pyrimidin-2-yl)amino]-3-methoxy-phenyl]methyl]azetidin-3-ol;
(3S)-1-[[4-[(5-chloro-4-imidazo[1,2-a]pyridin-3-yl-pyrimidin-2-yl)amino]-3-methoxy-phenyl]methyl]piperidin-3-ol;
(3R)-1-[[4-[(5-chloro-4-imidazo[1,2-a]pyridin-3-yl-pyrimidin-2-yl)amino]-3-methoxy-phenyl]methyl]pyrrolidin-3-ol;
(2S)-2-[[4-[(5-chloro-4-imidazo[1,2-a]pyridin-3-yl-pyrimidin-2-yl)amino]-3-methoxy-phenyl]methylamino]propan-1-ol;
5-chloro-4-imidazo[1,2-a]pyridin-3-yl-N-[2-methoxy-4-(morpholinomethyl)phenyl]pyrimidin-2-amine;
[4-[4-[(5-chloro-4-imidazo[1,2-a]pyridin-3-yl-pyrimidin-2-yl)amino]-3-methoxy-phenyl]piperazin-1-yl]-(1-hydroxycyclopropyl)methanone;
[4-[4-[(5-chloro-4-imidazo[1,2-a]pyridin-3-yl-pyrimidin-2-yl)amino]-3-methoxy-phenyl]piperazin-1-yl]-[(2S)-pyrrolidin-2-yl]methanone;
[4-[4-[(5-chloro-4-imidazo[1,2-a]pyridin-3-yl-pyrimidin-2-yl)amino]-3-methoxy-phenyl]piperazin-1-yl]-[(2R)-2-piperidyl]methanone;
[4-[4-[(5-chloro-4-imidazo[1,2-a]pyridin-3-yl-pyrimidin-2-yl)amino]-3-methoxy-phenyl]piperazin-1-yl]-[(2S)-2-piperidyl]methanone;
[4-[4-[(5-chloro-4-imidazo[1,2-a]pyridin-3-ylpyrimidin-2-yl)amino]-3-methoxyphenyl]piperazin-1-yl]-pyrrolidin-2-ylmethanone;
2-amino-1-[4-[4-[(5-chloro-4-imidazo[1,2-a]pyridin-3-yl-pyrimidin-2-yl)amino]-3-methoxy-phenyl]piperazin-1-yl]ethanone;
(2S)-2-amino-1-[4-[4-[(5-chloro-4-imidazo[1,2-a]pyridin-3-yl-pyrimidin-2-yl)amino]-3-methoxy-phenyl]piperazin-1-yl]propan-1-one;
(2S)-2-amino-1-[4-[4-[(5-chloro-4-imidazo[1,2-a]pyridin-3-yl-pyrimidin-2-yl)amino]-3-methoxy-phenyl]piperazin-1-yl]-3-methyl-butan-1-one;
(2S)-2-amino-1-[4-[4-[(5-chloro-4-imidazo[1,2-a]pyridin-3-yl-pyrimidin-2-yl)amino]-3-methoxy-phenyl]piperazin-1-yl]-3-hydroxy-propan-1-one;
(3S)-3-amino-4-[4-[4-[(5-chloro-4-imidazo[1,2-a]pyridin-3-yl-pyrimidin-2-yl)amino]-3-methoxy-phenyl]piperazin-1-yl]-4-oxo-butanamide;
[4-[4-[(5-chloro-4-imidazo[1,2-a]pyridin-3-yl-pyrimidin-2-yl)amino]-3-methoxy-phenyl]piperazin-1-yl]-[(2S,4R)-4-hydroxypyrrolidin-2-yl]methanone;
1-[4-[4-[(5-chloro-4-imidazo[1,2-a]pyridin-3-yl-pyrimidin-2-yl)amino]-3-methoxy-phenyl]piperazin-1-yl]-2-methylamino-ethanone;
1-[4-[4-[(5-chloro-4-imidazo[1,2-a]pyridin-3-yl-pyrimidin-2-yl)amino]-3-methoxy-phenyl]piperazin-1-yl]-2-ethylamino-ethanone;
(2R)-2-amino-1-[4-[4-[(5-chloro-4-imidazo[1,2-a]pyridin-3-yl-pyrimidin-2-yl)amino]-3-methoxy-phenyl]piperazin-1-yl]-3-hydroxy-propan-1-one;
(2S)-2-amino-1-[4-[4-[(5-chloro-4-imidazo[1,2-a]pyridin-3-yl-pyrimidin-2-yl)amino]-3-methoxy-phenyl]piperazin-1-yl]-4-hydroxy-butan-1-one;
(2R)-2-amino-1-[4-[4-[(5-chloro-4-imidazo[1,2-a]pyridin-3-yl-pyrimidin-2-yl)amino]-3-methoxy-phenyl]piperazin-1-yl]propan-1-one;
5-[4-[4-[(5-chloro-4-imidazo[1,2-a]pyridin-3-yl-pyrimidin-2-yl)amino]-3-methoxy-phenyl]piperazine-1-carbonyl]pyrrolidin-2-one;
N-[2-[4-[4-[(5-chloro-4-imidazo[1,2-a]pyridin-3-yl-pyrimidin-2-yl)amino]-3-methoxy-phenyl]piperazin-1-yl]-2-oxo-ethyl]acetamide;
(2S)-1-[4-[4-[(5-chloro-4-imidazo[1,2-a]pyridin-3-yl-pyrimidin-2-yl)amino]-3-methoxy-phenyl]piperazin-1-yl]-2-methoxy-propan-1-one;
(2R)-1-[4-[4-[(5-chloro-4-imidazo[1,2-a]pyridin-3-yl-pyrimidin-2-yl)amino]-3-methoxy-phenyl]piperazin-1-yl]-2-hydroxy-propan-1-one;
(2R)-1-[4-[4-[(5-chloro-4-imidazo[1,2-a]pyridin-3-yl-pyrimidin-2-yl)amino]-3-methoxy-phenyl]piperazin-1-yl]-2-methoxy-propan-1-one;
1-[4-[4-[(5-chloro-4-imidazo[1,2-a]pyridin-3-yl-pyrimidin-2-yl)amino]-3-methoxy-phenyl]piperazin-1-yl]-2-hydroxy-2-methyl-propan-1-one;
N-[4-(azetidin-3-yloxy)-2-methoxy-phenyl]-5-chloro-4-imidazo[1,2-a]pyridin-3-yl-pyrimidin-2-amine;
(2S)-1-[3-[4-[(5-chloro-4-imidazo[1,2-a]pyridin-3-yl-pyrimidin-2-yl)amino]-3-methoxy-phenoxy]azetidin-1-yl]-2-hydroxy-propan-1-one;
(2S)-1-[3-[4-[(5-chloro-4-imidazo[1,2-a]pyridin-3-yl-pyrimidin-2-yl)amino]-3-methoxy-phenoxy]azetidin-1-yl]-2-methoxy-propan-1-one;
1-[3-[4-[(5-chloro-4-imidazo[1,2-a]pyridin-3-yl-pyrimidin-2-yl)amino]-3-methoxy-phenoxy]azetidin-1-yl]-2-hydroxy-ethanone;
(2R)-1-[3-[4-[(5-chloro-4-imidazo[1,2-a]pyridin-3-yl-pyrimidin-2-yl)amino]-3-methoxy-phenoxy]azetidin-1-yl]-2-methoxy-propan-1-one;
[3-[4-[(5-chloro-4-imidazo[1,2-a]pyridin-3-yl-pyrimidin-2-yl)amino]-3-methoxy-phenoxy]azetidin-1-yl]-(1-hydroxycyclopropyl)methanone;
(2R)-1-[3-[4-[(5-chloro-4-imidazo[1,2-a]pyridin-3-yl-pyrimidin-2-yl)amino]-3-methoxy-phenoxy]azetidin-1-yl]-2-hydroxy-propan-1-one;
(2R)-1-[4-[4-[(5-chloro-4-imidazo[1,2-a]pyridin-3-yl-pyrimidin-2-yl)amino]-3-methoxy-phenyl]-1-piperidyl]-2-methoxy-propan-1-one;
(2R)-1-[4-[4-[(5-chloro-4-imidazo[1,2-a]pyridin-3-yl-pyrimidin-2-yl)amino]-3-methoxy-phenyl]-1-piperidyl]-2-hydroxy-propan-1-one;
(2S)-1-[4-[4-[(5-chloro-4-imidazo[1,2-a]pyridin-3-yl-pyrimidin-2-yl)amino]-3-methoxy-phenyl]-1-piperidyl]-2-methoxy-propan-1-one;
(2S)-1-[4-[4-[(5-chloro-4-imidazo[1,2-a]pyridin-3-yl-pyrimidin-2-yl)amino]-3-methoxy-phenyl]-1-piperidyl]-2-hydroxy-propan-1-one;
[4-[(5-chloro-4-imidazo[1,2-a]pyridin-3-yl-pyrimidin-2-yl)amino]-3-methoxy-phenyl]-[(1S,4S)-2,5-diazabicyclo[2.2.1]heptan-5-yl]methanone;
[4-[(5-chloro-4-imidazo[1,2-a]pyridin-3-yl-pyrimidin-2-yl)amino]-3-methoxy-phenyl]-piperazin-1-yl-methanone;
[4-[(5-chloro-4-imidazo[1,2-a]pyridin-3-yl-pyrimidin-2-yl)amino]-3-methoxy-phenyl]-[(1S,4S)-5-methyl-2,5-diazabicyclo[2.2.1]heptan-2-yl]methanone;
[4-[(5-chloro-4-imidazo[1,2-a]pyridin-3-yl-pyrimidin-2-yl)amino]-3-methoxy-phenyl]-[4-(2-hydroxyethyl)piperazin-1-yl]methanone;
4-[(5-chloro-4-imidazo[1,2-a]pyridin-3-yl-pyrimidin-2-yl)amino]-N-(2-hydroxyethyl)-3-methoxy-N-methyl-benzamide;
2-[4-[(5-chloro-4-imidazo[1,2-a]pyridin-3-yl-pyrimidin-2-yl)amino]-3-methoxy-phenyl]-1-piperazin-1-yl-ethanone;
2-[4-[(5-chloro-4-imidazo[1,2-a]pyridin-3-yl-pyrimidin-2-yl)amino]-3-methoxy-phenyl]-N-pyrrolidin-3-yl-acetamide;
2-[4-[(5-chloro-4-imidazo[1,2-a]pyridin-3-yl-pyrimidin-2-yl)amino]-3-methoxy-phenyl]-1-morpholino-ethanone;
2-[4-[(5-chloro-4-imidazo[1,2-a]pyridin-3-yl-pyrimidin-2-yl)amino]-3-methoxy-phenyl]-1-[4-(2-hydroxyethyl)piperazin-1-yl]ethanone;
2-[4-[(5-chloro-4-imidazo[1,2-a]pyridin-3-yl-pyrimidin-2-yl)amino]-3-methoxy-phenyl]-1-[(3S)-3-hydroxypyrrolidin-1-yl]ethanone;
1-[4-[4-[(5-chloro-4-imidazo[1,2-a]pyridin-3-yl-pyrimidin-2-yl)amino]-3-methoxy-phenyl]piperazin-1-yl]-2-methoxy-ethanone;
1-[4-[4-[(5-chloro-4-imidazo[1,2-a]pyridin-3-yl-pyrimidin-2-yl)amino]-3-methoxy-phenyl]piperazin-1-yl]-2-hydroxy-ethanone;
(2S)-1-[4-[4-[(5-chloro-4-imidazo[1,2-a]pyridin-3-yl-pyrimidin-2-yl)amino]-3-methoxy-phenyl]piperazin-1-yl]-2-hydroxy-propan-1-one;
1-[4-[4-[(5-chloro-4-imidazo[1,2-a]pyridin-3-yl-pyrimidin-2-yl)amino]-3-methoxy-phenyl]piperazin-1-yl]propan-1-one;
[4-[4-[(5-chloro-4-imidazo[1,2-a]pyridin-3-ylpyrimidin-2-yl)amino]-3-methoxyphenyl]piperazin-1-yl]-cyclopropylmethanone;
methyl 4-[4-[(5-chloro-4-imidazo[1,2-a]pyridin-3-yl-pyrimidin-2-yl)amino]-3-methoxy-phenyl]piperazine-1-carboxylate;
2-methoxyethyl 4-[4-[(5-chloro-4-imidazo[1,2-a]pyridin-3-yl-pyrimidin-2-yl)amino]-3-methoxy-phenyl]piperazine-1-carboxylate;
[4-[4-[(5-chloro-4-imidazo[1,2-a]pyridin-3-yl-pyrimidin-2-yl)amino]-3-methoxy-phenyl]piperazin-1-yl]-morpholino-methanone;
4-[4-[(5-chloro-4-imidazo[1,2-a]pyridin-3-yl-pyrimidin-2-yl)amino]-3-methoxy-phenyl]-N-(2-hydroxyethyl)-N-methyl-piperazine-1-carboxamide;
4-[4-[(5-chloro-4-imidazo[1,2-a]pyridin-3-yl-pyrimidin-2-yl)amino]-3-methoxy-phenyl]-N,N-dimethyl-piperazine-1-carboxamide;
[4-[4-[(5-chloro-4-imidazo[1,2-a]pyridin-3-yl-pyrimidin-2-yl)amino]-3-methoxy-phenyl]piperazin-1-yl]-piperazin-1-yl-methanone;
5-chloro-4-imidazo[1,2-a]pyridin-3-yl-N-[2-methoxy-4-[4-(2-methylsulfonylethyl)piperazin-1-yl]phenyl]pyrimidin-2-amine;
3-[4-[4-[(5-chloro-4-imidazo[1,2-a]pyridin-3-yl-pyrimidin-2-yl)amino]-3-methoxy-phenyl]piperazin-1-yl]propanenitrile;
(2S)-1-[4-[4-[(5-chloro-4-imidazo[1,2-a]pyridin-3-yl-pyrimidin-2-yl)amino]-3-methoxy-phenyl]piperazin-1-yl]propan-2-ol;
(2R)-1-[4-[4-[(5-chloro-4-imidazo[1,2-a]pyridin-3-yl-pyrimidin-2-yl)amino]-3-methoxy-phenyl]piperazin-1-yl]propan-2-ol;
1-[4-[4-[(5-chloro-4-imidazo[1,2-a]pyridin-3-yl-pyrimidin-2-yl)amino]-3-methoxy-phenyl]piperazin-1-yl]-2-methyl-propan-2-ol;
(2R)-3-[4-[4-[(5-chloro-4-imidazo[1,2-a]pyridin-3-yl-pyrimidin-2-yl)amino]-3-methoxy-phenyl]piperazin-1-yl]propane-1,2-diol;
4-[4-[(5-chloro-4-imidazo[1,2-a]pyridin-3-yl-pyrimidin-2-yl)amino]-3-methoxy-phenyl]piperazine-1-carboxamide;
4-[3-[(5-chloro-4-imidazo[1,2-a]pyridin-3-yl-pyrimidin-2-yl)amino]-4-methoxy-phenyl]piperazine-1-carboxamide;
(2S)-1-[4-[4-[(5-chloro-4-imidazo[1,2-a]pyridin-3-yl-pyrimidin-2-yl)amino]-3-methoxy-phenyl]piperazin-1-yl]-2,3-dihydroxy-propan-1-one;
4-[4-[(5-chloro-4-imidazo[1,2-a]pyridin-3-yl-pyrimidin-2-yl)amino]-3-methoxy-phenyl]-N,N-dimethyl-piperidine-1-carboxamide;
4-[4-[(5-chloro-4-imidazo[1,2-a]pyridin-3-yl-pyrimidin-2-yl)amino]-3-methoxy-phenyl]-N-(2-hydroxyethyl)-N-methyl-piperidine-1-carboxamide;
1-[4-[4-[(5-chloro-4-imidazo[1,2-a]pyridin-3-yl-pyrimidin-2-yl)amino]-3-methoxy-phenyl]-2-methyl-1-piperidyl]ethanone;
5-chloro-4-imidazo[1,2-a]pyridin-3-yl-N-[2-methoxy-4-(2-methyl-4-piperidyl)phenyl]pyrimidin-2-amine;
1-[3-[4-[(5-chloro-4-imidazo[1,2-a]pyridin-3-yl-pyrimidin-2-yl)amino]-3-methoxy-phenoxy]azetidin-1-yl]ethanone;
(2S)-1-[3-[4-[(5-chloro-4-imidazo[1,2-a]pyridin-3-yl-pyrimidin-2-yl)amino]-3-methoxy-phenoxy]azetidin-1-yl]propan-2-ol;
(2R)-1-[3-[4-[(5-chloro-4-imidazo[1,2-a]pyridin-3-yl-pyrimidin-2-yl)amino]-3-methoxy-phenoxy]azetidin-1-yl]propan-2-ol;
1-[3-[4-[(5-chloro-4-imidazo[1,2-a]pyridin-3-yl-pyrimidin-2-yl)amino]-3-methoxy-phenoxy]azetidin-1-yl]-2-methyl-propan-2-ol;
2-[3-[4-[(5-chloro-4-imidazo[1,2-a]pyridin-3-yl-pyrimidin-2-yl)amino]-3-methoxy-phenoxy]azetidin-1-yl]ethanol;
2-[4-[4-[(5-chloro-4-imidazo[1,2-a]pyridin-3-yl-pyrimidin-2-yl)amino]-3-methoxy-phenyl]piperazin-1-yl]-N,N-dimethyl-acetamide;
2-[4-[4-[(5-chloro-4-imidazo[1,2-a]pyridin-3-yl-pyrimidin-2-yl)amino]-3-methoxy-phenyl]piperazin-1-yl]-N-methyl-acetamide;
2-[4-[4-[(5-chloro-4-imidazo[1,2-a]pyridin-3-yl-pyrimidin-2-yl)amino]-3-methoxy-phenyl]piperazin-1-yl]-N-(2-hydroxyethyl)-N-methyl-acetamide;
3-[4-[(5-chloro-4-imidazo[1,2-a]pyridin-3-yl-pyrimidin-2-yl)amino]-3-methoxy-phenoxy]-N-(2-hydroxyethyl)-N-methyl-azetidine-1-carboxamide;
3-[4-[(5-chloro-4-imidazo[1,2-a]pyridin-3-yl-pyrimidin-2-yl)amino]-3-methoxy-phenoxy]-N,N-dimethyl-azetidine-1-carboxamide;
2-[4-[(5-chloro-4-imidazo[1,2-a]pyridin-3-yl-pyrimidin-2-yl)amino]-3-methoxy-phenyl]propane-1,3-diol;
N-[4-[(3R)-3-aminopyrrolidin-1-yl]-2-methoxy-phenyl]-5-chloro-4-imidazo[1,2-a]pyridin-3-yl-pyrimidin-2-amine;
1-[4-[4-[(5-fluoro-4-imidazo[1,2-a]pyridin-3-yl-pyrimidin-2-yl)amino]-3-methoxy-phenyl]piperazin-1-yl]ethanone;
5-fluoro-4-imidazo[1,2-a]pyridin-3-yl-N-(2-methoxy-4-piperazin-1-yl-phenyl)pyrimidin-2-amine;
4-[4-[(5-fluoro-4-imidazo[1,2-a]pyridin-3-yl-pyrimidin-2-yl)amino]-3-methoxy-phenyl]-N,N-dimethyl-piperazine-1-sulfonamide;
5-fluoro-4-imidazo[1,2-a]pyridin-3-yl-N-(2-methoxy-5-piperazin-1-yl-phenyl)pyrimidin-2-amine;
N-[4-[(3R)-3-dimethylaminopyrrolidin-1-yl]-2-methoxy-phenyl]-5-fluoro-4-imidazo[1,2-a]pyridin-3-yl-pyrimidin-2-amine;
5-fluoro-4-imidazo[1,2-a]pyridin-3-yl-N-[2-methoxy-5-(4-methoxy-1-piperidyl)phenyl]pyrimidin-2-amine;
1-[4-[4-[(5-fluoro-4-imidazo[1,2-a]pyridin-3-yl-pyrimidin-2-yl)amino]-3-methoxy-phenyl]-1-piperidyl]ethanone;
5-fluoro-4-imidazo[1,2-a]pyridin-3-yl-N-(2-methoxy-4-morpholino-phenyl)pyrimidin-2-amine;
5-fluoro-4-imidazo[1,2-a]pyridin-3-yl-N-(2-methoxy-4-pyrrolidin-1-yl-phenyl)pyrimidin-2-amine;
2-[4-[4-[(5-fluoro-4-imidazo[1,2-a]pyridin-3-yl-pyrimidin-2-yl)amino]-3-methoxy-phenyl]piperazin-1-yl]ethanol;
5-fluoro-4-imidazo[1,2-a]pyridin-3-yl-N-[2-methoxy-4-[4-(2-methoxyethyl)piperazin-1-yl]phenyl]pyrimidin-2-amine;
(3S)-1-[4-[(5-fluoro-4-imidazo[1,2-a]pyridin-3-yl-pyrimidin-2-yl)amino]-3-methoxy-phenyl]pyrrolidin-3-ol;
5-fluoro-4-imidazo[1,2-a]pyridin-3-yl-N-[2-methoxy-4-(1,4-oxazepan-4-yl)phenyl]pyrimidin-2-amine;
(3R)-1-[4-[(5-fluoro-4-imidazo[1,2-a]pyridin-3-yl-pyrimidin-2-yl)amino]-3-methoxy-phenyl]pyrrolidin-3-ol;
5-fluoro-4-imidazo[1,2-a]pyridin-3-yl-N-[2-methoxy-4-(4-piperidyl)phenyl]pyrimidin-2-amine;
5-fluoro-4-imidazo[1,2-a]pyridin-3-yl-N-[2-methoxy-4-[[(2R)-pyrrolidin-2-yl]methoxy]phenyl]pyrimidin-2-amine;
4-[4-[(5-fluoro-4-imidazo[1,2-a]pyridin-3-yl-pyrimidin-2-yl)amino]-3-methoxy-phenyl]piperazin-2-one;
(2S)-1-[4-[4-[(5-fluoro-4-imidazo[1,2-a]pyridin-3-yl-pyrimidin-2-yl)amino]-3-methoxy-phenyl]piperazin-1-yl]propan-2-ol;
(2R)-1-[4-[4-[(5-fluoro-4-imidazo[1,2-a]pyridin-3-yl-pyrimidin-2-yl)amino]-3-methoxy-phenyl]piperazin-1-yl]propan-2-ol;
1-[4-[4-[(5-fluoro-4-imidazo[1,2-a]pyridin-3-yl-pyrimidin-2-yl)amino]-3-methoxy-phenyl]piperazin-1-yl]-2-methyl-propan-2-ol;
1-[4-[4-[(4-imidazo[1,2-a]pyridin-3-yl-5-methoxy-pyrimidin-2-yl)amino]-3-methoxy-phenyl]piperazin-1-yl]ethanone;
4-imidazo[1,2-a]pyridin-3-yl-5-methoxy-N-(2-methoxy-4-piperazin-1-yl-phenyl)pyrimidin-2-amine;
1-[4-[4-[(4-imidazo[1,2-a]pyridin-3-yl-5-methoxy-pyrimidin-2-yl)amino]-3-methoxy-phenyl]-1-piperidyl]ethanone;
2-[4-[4-[(4-imidazo[1,2-a]pyridin-3-yl-5-methoxy-pyrimidin-2-yl)amino]-3-methoxy-phenyl]piperazin-1-yl]ethanol;
4-imidazo[1,2-a]pyridin-3-yl-5-methoxy-N-[2-methoxy-4-[[(2R)-pyrrolidin-2-yl]methoxy]phenyl]pyrimidin-2-amine;
1-[4-[4-[(4-imidazo[1,2-a]pyridin-3-yl-5-methyl-pyrimidin-2-yl)amino]-3-methoxy-phenyl]piperazin-1-yl]ethanone;
4-imidazo[1,2-a]pyridin-3-yl-N-(2-methoxy-4-piperazin-1-yl-phenyl)-5-methyl-pyrimidin-2-amine;
1-[4-[4-[(4-imidazo[1,2-a]pyridin-3-yl-5-methyl-pyrimidin-2-yl)amino]-3-methoxy-phenyl]-1-piperidyl]ethanone;
4-imidazo[1,2-a]pyridin-3-yl-N-(2-methoxy-4-morpholino-phenyl)-5-methyl-pyrimidin-2-amine;
2-[4-[4-[(4-imidazo[1,2-a]pyridin-3-yl-5-methyl-pyrimidin-2-yl)amino]-3-methoxy-phenyl]piperazin-1-yl]ethanol;
4-imidazo[1,2-a]pyridin-3-yl-N-[2-methoxy-4-[[(2R)-pyrrolidin-2-yl]methoxy]phenyl]-5-methyl-pyrimidin-2-amine;
1-[4-[4-[(4-imidazo[1,2-a]pyridin-3-yl-5-methyl-pyrimidin-2-yl)amino]-3-methoxy-phenoxy]-1-piperidyl]ethanone;
(2S)-1-[4-[4-[(4-imidazo[1,2-a]pyridin-3-yl-5-methyl-pyrimidin-2-yl)amino]-3-methoxy-phenyl]piperazin-1-yl]propan-2-ol;
(2R)-1-[4-[4-[(4-imidazo[1,2-a]pyridin-3-yl-5-methyl-pyrimidin-2-yl)amino]-3-methoxy-phenyl]piperazin-1-yl]propan-2-ol;
1-[4-[4-[(4-imidazo[1,2-a]pyridin-3-yl-5-methyl-pyrimidin-2-yl)amino]-3-methoxy-phenyl]piperazin-1-yl]-2-methyl-propan-2-ol;
1-[4-[4-[[4-imidazo[1,2-a]pyridin-3-yl-5-(trifluoromethyl)pyrimidin-2-yl]amino]-3-methoxy-phenyl]piperazin-1-yl]ethanone;
1-[4-[4-[[4-imidazo[1,2-a]pyridin-3-yl-5-(trifluoromethyl)pyrimidin-2-yl]amino]-3-methoxy-phenyl]-1-piperidyl]ethanone;
2-[4-[4-[[4-imidazo[1,2-a]pyridin-3-yl-5-(trifluoromethyl)pyrimidin-2-yl]amino]-3-methoxy-phenyl]piperazin-1-yl]ethanol;
1-[4-[4-[[4-imidazo[1,2-a]pyridin-3-yl-5-(trifluoromethyl)pyrimidin-2-yl]amino]-3-methoxy-phenoxy]-1-piperidyl]ethanone;
1-[4-[(5-fluoro-4-imidazo[1,2-a]pyridin-3-yl-pyrimidin-2-yl)amino]-3-methoxy-phenyl]piperidin-4-ol;
5-fluoro-4-imidazo[1,2-a]pyridin-3-yl-N-[2-methoxy-5-(4-piperidyl)phenyl]pyrimidin-2-amine;
5-fluoro-4-imidazo[1,2-a]pyridin-3-yl-N-(2-methoxy-4-tetrahydropyran-4-yl-phenyl)pyrimidin-2-amine;
1-[(2R)-2-[[4-[(4-imidazo[1,2-a]pyridin-3-yl-5-methoxy-pyrimidin-2-yl)amino]-3-methoxy-phenoxy]methyl]pyrrolidin-1-yl]ethanone;
2-[4-[4-[(5-bromo-4-imidazo[1,2-a]pyridin-3-yl-pyrimidin-2-yl)amino]-3-methoxy-phenyl]piperazin-1-yl]ethanol;
2-[[4-[4-(2-hydroxyethyl)piperazin-1-yl]-2-methoxy-phenyl]amino]-4-imidazo[1,2-a]pyridin-3-yl-pyrimidine-5-carbonitrile;
1-[4-[4-[(5-bromo-4-imidazo[1,2-a]pyridin-3-yl-pyrimidin-2-yl)amino]-3-methoxy-phenyl]piperazin-1-yl]ethanone;
2-[[4-(4-acetylpiperazin-1-yl)-2-methoxy-phenyl]amino]-4-imidazo[1,2-a]pyridin-3-yl-pyrimidine-5-carbonitrile;
5-fluoro-4-imidazo[1,2-a]pyridin-3-yl-N-[2-methoxy-4-(4-methylpiperazin-1-yl)phenyl]pyrimidin-2-amine;
N-[4-(4-ethylpiperazin-1-yl)-2-methoxyphenyl]-5-fluoro-4-imidazo[1,2-a]pyridin-3-ylpyrimidin-2-amine;
5-bromo-4-imidazo[1,2-a]pyridin-3-yl-N-[2-methoxy-4-(4-methylpiperazin-1-yl)phenyl]pyrimidin-2-amine;
5-bromo-N-[2-ethoxy-4-(4-methylpiperazin-1-yl)phenyl]-4-imidazo[1,2-a]pyridin-3-yl-pyrimidin-2-amine;
5-bromo-4-imidazo[1,2-a]pyridin-3-yl-N-[2-methoxy-4-(1-methylpyrrolidin-3-yl)oxy-phenyl]pyrimidin-2-amine;
5-bromo-N-(4,5-dimethoxy-2-methyl-phenyl)-4-imidazo[1,2-a]pyridin-3-yl-pyrimidin-2-amine;
1-[4-[4-[[5-chloro-4-[7-(hydroxymethyl)imidazo[1,2-a]pyridin-3-yl]pyrimidin-2-yl]amino]-3-methoxy-phenyl]piperazin-1-yl]ethanone;
1-[4-[4-[[4-(6-bromoimidazo[1,2-a]pyridin-3-yl)-5-chloropyrimidin-2-yl]amino]-3-methoxyphenyl]piperazin-1-yl]ethanone;
3-[2-[[4-(4-acetylpiperazin-1-yl)-2-methoxyphenyl]amino]-5-chloropyrimidin-4-yl]imidazo[2,1-f]pyridine-6-carbonitrile
1-[4-[4-[[5-chloro-4-(6,8-difluoroimidazo[1,2-a]pyridin-3-yl)pyrimidin-2-yl]amino]-3-methoxyphenyl]piperazin-1-yl]ethanone;
1-[4-[4-[[5-chloro-4-(6-methylimidazo[1,2-a]pyridin-3-yl)pyrimidin-2-yl]amino]-3-methoxyphenyl]piperazin-1-yl]ethanone;
1-[4-[4-[[5-chloro-4-(6-dimethylaminoimidazo[1,2-a]pyridin-3-yl)pyrimidin-2-yl]amino]-3-methoxyphenyl]piperazin-1-yl]ethanone;
1-[4-[4-[[5-chloro-4-(6-methoxyimidazo[1,2-a]pyridin-3-yl)pyrimidin-2-yl]amino]-3-methoxyphenyl]piperazin-1-yl]ethanone;
3-[2-[[4-(4-acetylpiperazin-1-yl)-2-methoxyphenyl]amino]-5-chloropyrimidin-4-yl]imidazo[1,2-a]pyridine-7-carbonitrile;
1-[4-[4-[[5-chloro-4-(7-ethylimidazo[1,2-a]pyridin-3-yl)pyrimidin-2-yl]amino]-3-methoxyphenyl]piperazin-1-yl]ethanone;
1-[4-[4-[[5-chloro-4-(7-fluoroimidazo[1,2-a]pyridin-3-yl)pyrimidin-2-yl]amino]-3-methoxyphenyl]piperazin-1-yl]ethanone;
1-[4-[4-[[5-chloro-4-(7-methylimidazo[1,2-a]pyridin-3-yl)pyrimidin-2-yl]amino]-3-methoxyphenyl]piperazin-1-yl]ethanone;
1-[4-[4-[[5-chloro-4-(7-methoxyimidazo[1,2-a]pyridin-3-yl)pyrimidin-2-yl]amino]-3-methoxyphenyl]piperazin-1-yl]ethanone;
1-[4-[4-[[5-chloro-4-[8-(hydroxymethyl)imidazo[1,2-a]pyridin-3-yl]pyrimidin-2-yl]amino]-3-methoxyphenyl]piperazin-1-yl]ethanone;
3-[2-[[4-(4-acetylpiperazin-1-yl)-2-methoxyphenyl]amino]-5-chloropyrimidin-4-yl]imidazo[1,2-a]pyridine-8-carbonitrile;
1-[4-[4-[[5-chloro-4-(8-fluoroimidazo[1,2-a]pyridin-3-yl)pyrimidin-2-yl]amino]-3-methoxyphenyl]piperazin-1-yl]ethanone;
1-[4-[4-[[5-chloro-4-(8-methylimidazo[1,2-a]pyridin-3-yl)pyrimidin-2-yl]amino]-3-methoxyphenyl]piperazin-1-yl]ethanone;
1-[4-[4-[[5-chloro-4-(8-methoxyimidazo[1,2-a]pyridin-3-yl)pyrimidin-2-yl]amino]-3-methoxyphenyl]piperazin-1-yl]ethanone;
1-[4-[4-[[5-chloro-4-(6-fluoroimidazo[1,2-a]pyridin-3-yl)pyrimidin-2-yl]amino]-3-methoxy-phenyl]piperazin-1-yl]ethanone;
1-[4-[4-[(5-chloro-4-pyrazolo[1,5-a]pyridin-3-yl-pyrimidin-2-yl)amino]-3-methoxy-phenyl]piperazin-1-yl]ethanone;
4-(5-chloro-4-(imidazo[1,2-a]pyridin-3-yl)pyrimidin-2-ylamino)-3-methoxybenzaldehyde;
4-(5-chloro-4-(imidazo[1,2-a]pyridin-3-yl)pyrimidin-2-ylamino)-3-methoxybenzoic acid; and
2-(4-(5-chloro-4-(imidazo[1,2-a]pyridin-3-yl)pyrimidin-2-ylamino)-3-methoxyphenyl)acetic acid;
   and pharmaceutically acceptable salts thereof.

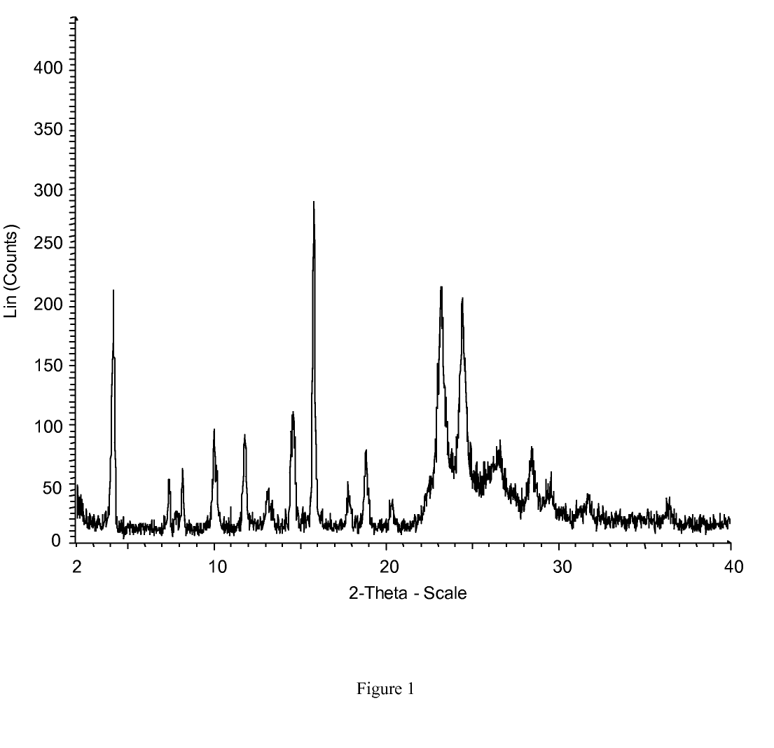

The present invention also provides a crystalline form A of 1-[4-[4-[(5-chloro-4-imidazo[1,2-a]pyridin-3-yl-pyrimidin-2-yl)amino]-3-methoxy-phenyl]piperazin-1-yl]ethanone. There is also provided a crystalline form A of 1-[4-[4-[(5-chloro-4-imidazo[1,2-a]pyridin-3-yl-pyrimidin-2-yl)amino]-3-methoxy-phenyl]piperazin-1-yl]ethanone (see Example 1) characterised in providing at least one of the following 20 values measured using CuKa radiation: 15.8 and 23.2. Form A of 1-[4-[4-[(5-chloro-4-imidazo[1,2-a]pyridin-3-yl-pyrimidin-2-yl)amino]-3-methoxy-phenyl]piperazin-1-yl]ethanone is characterised in providing an X-ray powder diffraction pattern substantially as shown in FIG. 1. The ten most prominent peaks are shown in the following table:

TABLE 1
Ten most Prominent X-Ray Powder Diffraction peaks for 1-[4-[4-
[(5-chloro-4-imidazo[1,2-a]pyridin-3-yl-pyrimidin-2-yl)amino]-3-
methoxy-phenyl]piperazin-1-yl]ethanone
Form A

| Angle 2- | | Relative |
|---|---|---|
| Theta (2θ) | Intensity % | Intensity |
| 15.758 | 100.0 | vs |
| 23.182 | 75.1 | vs |
| 24.423 | 71.6 | vs |
| 4.090 | 59.9 | vs |
| 14.537 | 36.0 | vs |
| 11.754 | 31.5 | vs |
| 9.975 | 29.1 | vs |
| 28.420 | 28.0 | vs |
| 26.580 | 27.7 | vs |
| 18.803 | 27.0 | vs |

vs = very strong,
s = strong

According to the present invention there is provided a crystalline form, Form A, of 1-[4-[4-[(5-chloro-4-imidazo[1,2-a]pyridin-3-yl-pyrimidin-2-yl)amino]-3-methoxy-phenyl]piperazin-1-yl]ethanone which has an X-ray powder diffraction pattern with at least one specific peak at about 2-theta=15.8°.

According to the present invention there is provided a crystalline form, Form A, of 1-[4-[4-[(5-chloro-4-imidazo[1,2-a]pyridin-3-yl-pyrimidin-2-yl)amino]-3-methoxy-phenyl]piperazin-1-yl]ethanone which has an X-ray powder diffraction pattern with at least one specific peak at about 2-theta=23.2°.

According to the present invention there is provided a crystalline form, Form A, of 1-[4-[4-[(5-chloro-4-imidazo[1,2-a]pyridin-3-yl-pyrimidin-2-yl)amino]-3-methoxy-phenyl]piperazin-1-yl]ethanone which has an X-ray powder diffraction pattern with at least two specific peaks at about 2-theta=15.8° and 23.2°.

According to the present invention there is provided a crystalline form, Form A, of 1-[4-[4-[(5-chloro-4-imidazo[1,2-a]pyridin-3-yl-pyrimidin-2-yl)amino]-3-methoxy-phenyl]piperazin-1-yl]ethanone which has an X-ray powder diffraction pattern with specific peaks at about 2-theta=15.8, 23.2, 24.4, 4.1, 14.5, 11.8, 10.0, 28.4, 26.6, and 18.8°.

According to the present invention there is provided crystalline form, Form A, of 1-[4-[4-[(5-chloro-4-imidazo[1,2-a]pyridin-3-yl-pyrimidin-2-yl)amino]-3-methoxy-phenyl]piperazin-1-yl]ethanone which has an X-ray powder diffraction pattern substantially the same as the X-ray powder diffraction pattern shown in FIG. 1.

According to the present invention there is provided crystalline form, Form A, of 1-[4-[4-[(5-chloro-4-imidazo[1,2-a]pyridin-3-yl-pyrimidin-2-yl)amino]-3-methoxy-phenyl]piperazin-1-yl]ethanone which has an X-ray powder diffraction pattern with at least one specific peak at 2-theta=15.8° plus or minus 0.5° 2-theta.

According to the present invention there is provided a crystalline form, Form A, of 1-[4-[4-[(5-chloro-4-imidazo[1,2-a]pyridin-3-yl-pyrimidin-2-yl)amino]-3-methoxy-phenyl]piperazin-1-yl]ethanone which has an X-ray powder diffraction pattern with at least one specific peak at 2-theta=23.2° plus or minus 0.5° 2-theta.

According to the present invention there is provided a crystalline form, Form A, of 1-[4-[4-[(5-chloro-4-imidazo[1,2-a]pyridin-3-yl-pyrimidin-2-yl)amino]-3-methoxy-phenyl]piperazin-1-yl]ethanone which has an X-ray powder diffraction pattern with at least two specific peaks at 2-theta=15.8° and 23.2° wherein said values may be plus or minus 0.5° 2-theta.

According to the present invention there is provided a crystalline form, Form A, of 1-[4-[4-[(5-chloro-4-imidazo[1,2-a]pyridin-3-yl-pyrimidin-2-yl)amino]-3-methoxy-phenyl]piperazin-1-yl]ethanone which has an X-ray powder diffraction pattern with specific peaks at 2-theta=15.8, 23.2, 24.4, 4.1, 14.5, 11.8, 10.0, 28.4, 26.6, and 18.8° wherein said values may be plus or minus 0.5° 2-theta.

According to the present invention there is provided a crystalline form, Form A, of 1-[4-[4-[(5-chloro-4-imidazo[1,2-a]pyridin-3-yl-pyrimidin-2-yl)amino]-3-methoxy-phenyl]piperazin-1-yl]ethanone which has an X-ray powder diffraction pattern with at least one specific peak at 2-theta=15.8°.

According to the present invention there is provided a crystalline form, Form A, of 1-[4-[4-[(5-chloro-4-imidazo[1,2-a]pyridin-3-yl-pyrimidin-2-yl)amino]-3-methoxy-phenyl]piperazin-1-yl]ethanone which has an X-ray powder diffraction pattern with at least one specific peak at 2-theta=23.2°.

According to the present invention there is provided a crystalline form, Form A, of 1-[4-[4-[(5-chloro-4-imidazo[1,2-a]pyridin-3-yl-pyrimidin-2-yl)amino]-3-methoxy-phenyl]piperazin-1-yl]ethanone which has an X-ray powder diffraction pattern with at least two specific peaks at 2-theta=15.8° and 23.2°.

According to the present invention there is provided crystalline form, Form A, of 1-[4-[4-[(5-chloro-4-imidazo[1,2-a]pyridin-3-yl-pyrimidin-2-yl)amino]-3-methoxy-phenyl]piperazin-1-yl]ethanone which has an X-ray powder diffraction pattern with specific peaks at 2-theta=15.8, 23.2, 24.4, 4.1, 14.5, 11.8, 10.0, 28.4, 26.6, and 18.8°.

According to the present invention there is provided crystalline form, Form A, of 1-[4-[4-[(5-chloro-4-imidazo[1,2-a]pyridin-3-yl-pyrimidin-2-yl)amino]-3-methoxy-phenyl]piperazin-1-yl]ethanone which has an X-ray powder diffraction pattern as shown in FIG. 1.

DSC analysis shows Form A of 1-[4-[4-[(5-chloro-4-imidazo[1,2-a]pyridin-3-yl-pyrimidin-2-yl)amino]-3-methoxy-phenyl]piperazin-1-yl]ethanone is a high melting solid with an onset of melting at about 187.1° C. and a peak at 193.1° C.

The Form A of 1-[4-[4-[(5-chloro-4-imidazo[1,2-a]pyridin-3-yl-pyrimidin-2-yl)amino]-3-methoxy-phenyl]piperazin-1-yl]ethanone provides X-ray powder diffraction patterns substantially the same as the X-ray powder diffraction patterns shown in FIG. 1 and has substantially the ten most prominent peaks (angle 2-theta values) shown in the Table above.

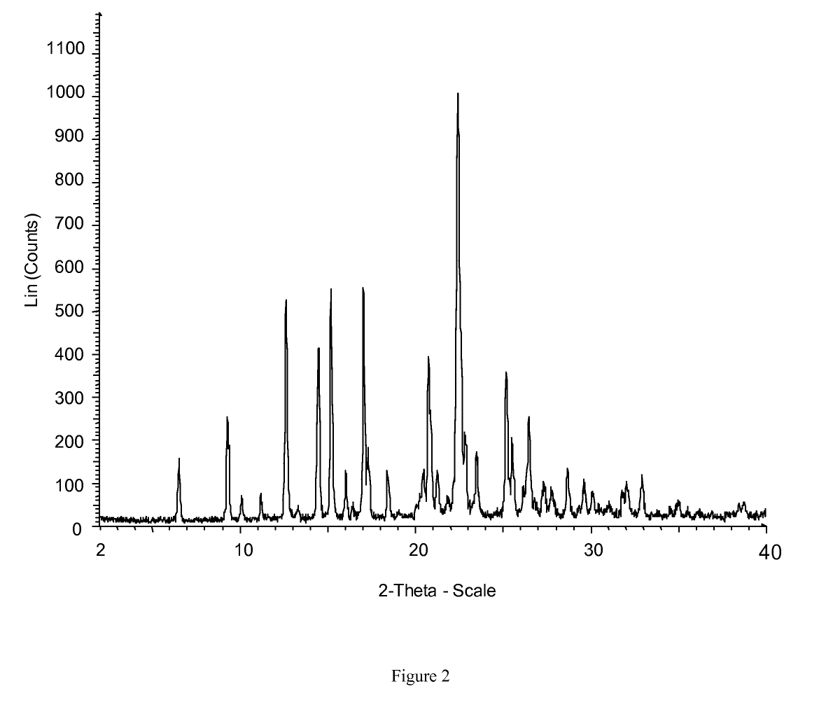

The present invention also provides a crystalline form A of 2-[4-[4-[(5-fluoro-4-imidazo[1,2-a]pyridin-3-yl-pyrimidin-2-yl)amino]-3-methoxy-phenyl]piperazin-1-yl]ethanol. There is also provided a crystalline form A of 2-[4-[4-[(5-fluoro-4-imidazo[1,2-a]pyridin-3-yl-pyrimidin-2-yl)amino]-3-methoxy-phenyl]piperazin-1-yl]ethanol (see Example 81) characterised in providing at least one of the following 2θ values measured using CuKa radiation: 22.4 and 17.0. Form A of 2-[4-[4-[(5-fluoro-4-imidazo[1,2-a]pyridin-3-yl-pyrimidin-2-yl)amino]-3-methoxy-phenyl]piperazin-1-yl]ethanol is characterised in providing an X-ray powder diffraction pattern substantially as shown in FIG. 2. The ten most prominent peaks are shown in the following table:

TABLE 2
Ten most Prominent X-Ray Powder Diffraction peaks for 2-[4-
[4-[(5-fluoro-4-imidazo[1,2-a]pyridin-3-yl-pyrimidin-2-yl)amino]-
3-methoxy-phenyl]piperazin-1-yl]ethanol
Form A

| Angle 2- | | Relative |
|---|---|---|
| Theta (2θ) | Intensity % | Intensity |
| 22.395 | 100.0 | vs |
| 17.003 | 54.8 | vs |
| 15.169 | 54.7 | vs |
| 12.584 | 52.2 | vs |
| 14.407 | 40.7 | vs |
| 20.745 | 38.9 | vs |
| 25.178 | 35.3 | vs |
| 9.241 | 25.1 | vs |
| 26.435 | 25.1 | vs |
| 22.801 | 21.6 | s |

vs = very strong,
s = strong

According to the present invention there is provided a crystalline form, Form A, of 2-[4-[4-[(5-fluoro-4-imidazo[1,2-a]pyridin-3-yl-pyrimidin-2-yl)amino]-3-methoxy-phenyl]piperazin-1-yl]ethanol which has an X-ray powder diffraction pattern with at least one specific peak at about 2-theta=22.4°.

According to the present invention there is provided a crystalline form, Form A, of 2-[4-[4-[(5-fluoro-4-imidazo[1,2-a]pyridin-3-yl-pyrimidin-2-yl)amino]-3-methoxy-phenyl]piperazin-1-yl]ethanol which has an X-ray powder diffraction pattern with at least one specific peak at about 2-theta=17.0°.

According to the present invention there is provided a crystalline form, Form A, of 2-[4-[4-[(5-fluoro-4-imidazo[1,2-a]pyridin-3-yl-pyrimidin-2-yl)amino]-3-methoxy-phenyl]piperazin-1-yl]ethanol which has an X-ray powder diffraction pattern with at least two specific peaks at about 2-theta=22.4° and 17.0°.

According to the present invention there is provided a crystalline form, Form A, of 2-[4-[4-[(5-fluoro-4-imidazo[1,2-a]pyridin-3-yl-pyrimidin-2-yl)amino]-3-methoxy-phenyl]piperazin-1-yl]ethanol which has an X-ray powder diffraction pattern with specific peaks at about 2-theta=22.4, 17.0, 15.2, 12.6, 14.4, 20.7, 25.2, 9.2, 26.4, and 22.8°.

According to the present invention there is provided crystalline form, Form A, of 2-[4-[4-[(5-fluoro-4-imidazo[1,2-a]pyridin-3-yl-pyrimidin-2-yl)amino]-3-methoxy-phenyl]piperazin-1-yl]ethanol which has an X-ray powder diffraction pattern substantially the same as the X-ray powder diffraction pattern shown in FIG. 2.

According to the present invention there is provided crystalline form, Form A, of 2-[4-[4-[(5-fluoro-4-imidazo[1,2-a]pyridin-3-yl-pyrimidin-2-yl)amino]-3-methoxy-phenyl]piperazin-1-yl]ethanol which has an X-ray powder diffraction pattern with at least one specific peak at 2-theta=22.4° plus or minus 0.5° 2-theta.

According to the present invention there is provided a crystalline form, Form A, of 2-[4-[4-[(5-fluoro-4-imidazo[1,2-a]pyridin-3-yl-pyrimidin-2-yl)amino]-3-methoxy-phenyl]piperazin-1-yl]ethanol which has an X-ray powder diffraction pattern with at least one specific peak at 2-theta=17.0° plus or minus 0.5° 2-theta.

According to the present invention there is provided a crystalline form, Form A, of 2-[4-[4-[(5-fluoro-4-imidazo[1,2-a]pyridin-3-yl-pyrimidin-2-yl)amino]-3-methoxy-phenyl]piperazin-1-yl]ethanol which has an X-ray powder diffraction pattern with at least two specific peaks at 2-theta=22.4° and 17.0° wherein said values may be plus or minus 0.5° 2-theta.

According to the present invention there is provided a crystalline form, Form A, of 2-[4-[4-[(5-fluoro-4-imidazo[1,2-a]pyridin-3-yl-pyrimidin-2-yl)amino]-3-methoxy-phenyl]piperazin-1-yl]ethanol which has an X-ray powder diffraction pattern with specific peaks at 2-theta=22.4, 17.0, 15.2, 12.6, 14.4, 20.7, 25.2, 9.2, 26.4, and 22.8° wherein said values may be plus or minus 0.5° 2-theta.

According to the present invention there is provided a crystalline form, Form A, of 2-[4-[4-[(5-fluoro-4-imidazo[1,2-a]pyridin-3-yl-pyrimidin-2-yl)amino]-3-methoxy-phenyl]piperazin-1-yl]ethanol which has an X-ray powder diffraction pattern with at least one specific peak at 2-theta=22.4°.

According to the present invention there is provided a crystalline form, Form A, of 2-[4-[4-[(5-fluoro-4-imidazo[1,2-a]pyridin-3-yl-pyrimidin-2-yl)amino]-3-methoxy-phenyl]piperazin-1-yl]ethanol which has an X-ray powder diffraction pattern with at least one specific peak at 2-theta=17.0°.

According to the present invention there is provided a crystalline form, Form A, of 2-[4-[4-[(5-fluoro-4-imidazo[1,2-a]pyridin-3-yl-pyrimidin-2-yl)amino]-3-methoxy-phenyl]piperazin-1-yl]ethanol which has an X-ray powder diffraction pattern with at least two specific peaks at 2-theta=22.4° and 17.0°.

According to the present invention there is provided crystalline form, Form A, of 2-[4-[4-[(5-fluoro-4-imidazo[1,2-a]pyridin-3-yl-pyrimidin-2-yl)amino]-3-methoxy-phenyl]piperazin-1-yl]ethanol which has an X-ray powder diffraction pattern with specific peaks at 2-theta=22.4, 17.0, 15.2, 12.6, 14.4, 20.7, 25.2, 9.2, 26.4, and 22.8°.

According to the present invention there is provided crystalline form, Form A, of 2-[4-[4-[(5-fluoro-4-imidazo[1,2-a]pyridin-3-yl-pyrimidin-2-yl)amino]-3-methoxy-phenyl]piperazin-1-yl]ethanol which has an X-ray powder diffraction pattern as shown in Figure A.

DSC analysis shows Form A of 2-[4-[4-[(5-fluoro-4-imidazo[1,2-a]pyridin-3-yl-pyrimidin-2-yl)amino]-3-methoxy-phenyl]piperazin-1-yl]ethanol is a high melting solid with an onset of melting at about 171.3° C. and a peak at 172.0° C.

The Form A of 2-[4-[4-[(5-fluoro-4-imidazo[1,2-a]pyridin-3-yl-pyrimidin-2-yl)amino]-3-methoxy-phenyl]piperazin-1-yl]ethanol provides X-ray powder diffraction patterns substantially the same as the X-ray powder diffraction patterns shown in FIG. 2 and has substantially the ten most prominent peaks (angle 2-theta values) shown in the Table above.

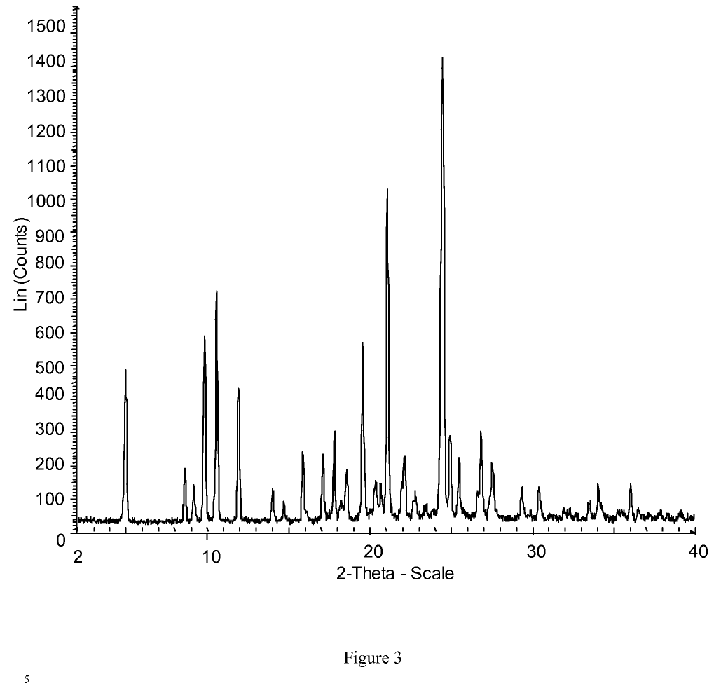

The present invention also provides a crystalline form A of 2-[4-[4-[(4-imidazo[1,2-a]pyridin-3-yl-5-methyl-pyrimidin-2-yl)amino]-3-methoxy-phenyl]piperazin-1-yl]ethanol. There is also provided a crystalline form A of 2-[4-[4-[(4-imidazo[1,2-a]pyridin-3-yl-5-methyl-pyrimidin-2-yl)amino]-3-methoxy-phenyl]piperazin-1-yl]ethanol (see Example 101) characterised in providing at least one of the following 2θ values measured using CuKa radiation: 24.5 and 21.1. Form A of 2-[4-[4-[(4-imidazo[1,2-a]pyridin-3-yl-5-methyl-pyrimidin-2-yl)amino]-3-methoxy-phenyl]piperazin-1-yl]ethanol is characterised in providing an X-ray powder diffraction pattern substantially as shown in FIG. 3. The ten most prominent peaks are shown in the following Table:

TABLE 3
Ten most Prominent X-Ray Powder Diffraction peaks for 2-
[4-[4-[(4-imidazo[1,2-a]pyridin-3-yl-5-methyl-pyrimidin-2-yl)amino]-
3-methoxy-phenyl]piperazin-1-yl]ethanol
Form A

| Angle 2- | | Relative |
|---|---|---|
| Theta (2θ) | Intensity % | Intensity |
| 24.450 | 100.0 | vs |
| 21.065 | 72.0 | vs |
| 10.506 | 50.4 | vs |
| 9.767 | 40.6 | vs |
| 19.525 | 39.3 | vs |
| 4.904 | 33.3 | vs |
| 11.868 | 29.6 | vs |
| 17.777 | 20.4 | s |
| 26.827 | 20.4 | s |
| 24.919 | 19.3 | s |

vs = very strong,
s = strong

According to the present invention there is provided a crystalline form, Form A, of 2-[4-[4-[(4-imidazo[1,2-a]pyridin-3-yl-5-methyl-pyrimidin-2-yl)amino]-3-methoxy-phenyl]piperazin-1-yl]ethanol which has an X-ray powder diffraction pattern with at least one specific peak at about 2-theta=24.5°.

According to the present invention there is provided a crystalline form, Form A, of 2-[4-[4-[(4-imidazo[1,2-a]pyridin-3-yl-5-methyl-pyrimidin-2-yl)amino]-3-methoxy-phenyl]piperazin-1-yl]ethanol which has an X-ray powder diffraction pattern with at least one specific peak at about 2-theta=21.1°.

According to the present invention there is provided a crystalline form, Form A, of 2-[4-[4-[(4-imidazo[1,2-a]pyridin-3-yl-5-methyl-pyrimidin-2-yl)amino]-3-methoxy-phenyl]piperazin-1-yl]ethanol which has an X-ray powder diffraction pattern with at least two specific peaks at about 2-theta=24.5° and 21.1°.

According to the present invention there is provided a crystalline form, Form A, of 2-[4-[4-[(4-imidazo[1,2-a]pyridin-3-yl-5-methyl-pyrimidin-2-yl)amino]-3-methoxy-phenyl]piperazin-1-yl]ethanol which has an X-ray powder diffraction pattern with specific peaks at about 2-theta=24.5, 21.1, 10.5, 9.8, 19.5, 4.9, 11.9, 17.8, 26.8, and 24.9°.

According to the present invention there is provided crystalline form, Form A, of 2-[4-[4-[(4-imidazo[1,2-a]pyridin-3-yl-5-methyl-pyrimidin-2-yl)amino]-3-methoxy-phenyl]piperazin-1-yl]ethanol which has an X-ray powder diffraction pattern substantially the same as the X-ray powder diffraction pattern shown in FIG. 3.

According to the present invention there is provided crystalline form, Form A, of 2-[4-[4-[(4-imidazo[1,2-a]pyridin-3-yl-5-methyl-pyrimidin-2-yl)amino]-3-methoxy-phenyl]piperazin-1-yl]ethanol which has an X-ray powder diffraction pattern with at least one specific peak at 2-theta=24.5° plus or minus 0.5° 2-theta.

According to the present invention there is provided a crystalline form, Form A, of 2-[4-[4-[(4-imidazo[1,2-a]pyridin-3-yl-5-methyl-pyrimidin-2-yl)amino]-3-methoxy-phenyl]piperazin-1-yl]ethanol which has an X-ray powder diffraction pattern with at least one specific peak at 2-theta=21.1° plus or minus 0.5° 2-theta.

According to the present invention there is provided a crystalline form, Form A, of 2-[4-[4-[(4-imidazo[1,2-a]pyridin-3-yl-5-methyl-pyrimidin-2-yl)amino]-3-methoxy-phenyl]piperazin-1-yl]ethanol which has an X-ray powder diffraction pattern with at least two specific peaks at 2-theta=24.5° and 21.1° wherein said values may be plus or minus 0.5° 2-theta.

According to the present invention there is provided a crystalline form, Form A, of 2-[4-[4-[(4-imidazo[1,2-a]pyridin-3-yl-5-methyl-pyrimidin-2-yl)amino]-3-methoxy-phenyl]piperazin-1-yl]ethanol which has an X-ray powder diffraction pattern with specific peaks at 2-theta=24.5, 21.1, 10.5, 9.8, 19.5, 4.9, 11.9, 17.8, 26.8, and 24.9° wherein said values may be plus or minus 0.5° 2-theta.

According to the present invention there is provided a crystalline form, Form A, of 2-[4-[4-[(4-imidazo[1,2-a]pyridin-3-yl-5-methyl-pyrimidin-2-yl)amino]-3-methoxy-phenyl]piperazin-1-yl]ethanol which has an X-ray powder diffraction pattern with at least one specific peak at 2-theta=24.5°.

According to the present invention there is provided a crystalline form, Form A, of 2-[4-[4-[(4-imidazo[1,2-a]pyridin-3-yl-5-methyl-pyrimidin-2-yl)amino]-3-methoxy-phenyl]piperazin-1-yl]ethanol which has an X-ray powder diffraction pattern with at least one specific peak at 2-theta=21.1°.

According to the present invention there is provided a crystalline form, Form A, of 2-[4-[4-[(4-imidazo[1,2-a]pyridin-3-yl-5-methyl-pyrimidin-2-yl)amino]-3-methoxy-phenyl]piperazin-1-yl]ethanol which has an X-ray powder diffraction pattern with at least two specific peaks at 2-theta=24.5° and 21.1°.

According to the present invention there is provided crystalline form, Form A, of 2-[4-[4-[(4-imidazo[1,2-a]pyridin-3-yl-5-methyl-pyrimidin-2-yl)amino]-3-methoxy-phenyl]piperazin-1-yl]ethanol which has an X-ray powder diffraction pattern with specific peaks at 2-theta=24.5, 21.1, 10.5, 9.8, 19.5, 4.9, 11.9, 17.8, 26.8, and 24.9°.

According to the present invention there is provided crystalline form, Form A, of 2-[4-[4-[(4-imidazo[1,2-a]pyridin-3-yl-5-methyl-pyrimidin-2-yl)amino]-3-methoxy-phenyl]piperazin-1-yl]ethanol which has an X-ray powder diffraction pattern as shown in FIG. 3.

DSC analysis shows Form A of 2-[4-[4-[(4-imidazo[1,2-a]pyridin-3-yl-5-methyl-pyrimidin-2-yl)amino]-3-methoxy-phenyl]piperazin-1-yl]ethanol is a high melting solid with an onset of melting at about 182.1° C. and a peak at about 184.1° C.

Form A of 2-[4-[4-[(4-imidazo[1,2-a]pyridin-3-yl-5-methyl-pyrimidin-2-yl)amino]-3-methoxy-phenyl]piperazin-1-yl]ethanol provides X-ray powder diffraction patterns substantially the same as the X-ray powder diffraction patterns shown in FIG. 3 and has substantially the ten most prominent peaks (angle 2-theta values) shown in the Table above.

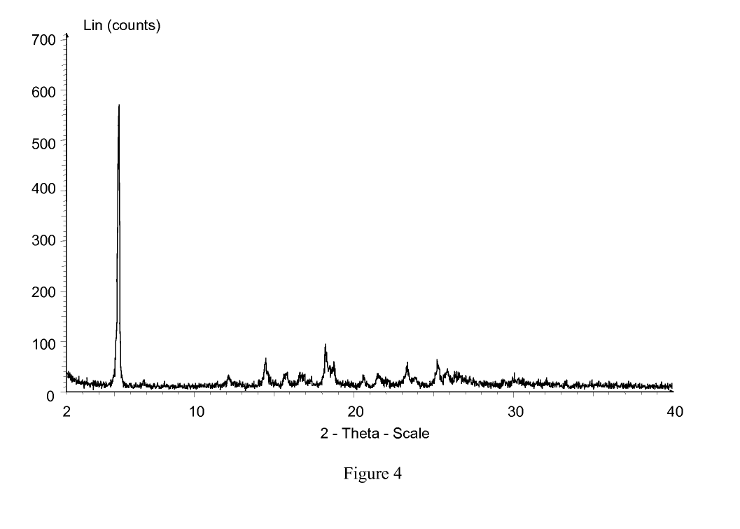

The present invention also provides a crystalline form A of a 2-[4-[4-[(5-fluoro-4-imidazo[1,2-a]pyridin-3-yl-pyrimidin-2-yl)amino]-3-methoxy-phenyl]piperazin-1-yl]ethanol hydrate. Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol is characterised in providing at least one of the following 2θ values measured using CuKa radiation: 5.2° and 18.2°. Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol Form A is characterised in providing an X-ray powder diffraction pattern, substantially as shown in FIG. 4. The ten most prominent peaks are shown in Table A:

TABLE 4
Ten most Prominent X-Ray Powder Diffraction peaks for Hydrate
Form A of 2-[4-[4-[(5-fluoro-4-imidazo[1,2-a]pyridin-3-yl-pyrimidin-
2-yl)amino]-3-methoxy-phenyl]piperazin-1-yl]ethanol

| Angle 2- | | Relative |
|---|---|---|
| Theta (2θ) | Intensity % | Intensity |
| 5.222 | 100.0 | vs |
| 18.244 | 16.3 | s |
| 18.744 | 10.2 | s |
| 14.449 | 10.0 | s |
| 23.366 | 10.0 | s |
| 25.290 | 9.3 | m |
| 25.867 | 7.7 | m |
| 16.638 | 6.5 | m |
| 21.538 | 6.0 | m |
| 15.754 | 6.0 | m |

vs = very strong
s = strong
m = medium

According to the present invention there is provided a crystalline form, Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol, which has an X-ray powder diffraction pattern with at least one specific peak at about 2-theta=5.2°.

According to the present invention there is provided a crystalline form, Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol, which has an X-ray powder diffraction pattern with at least one specific peak at about 2-theta=18.2°.

According to the present invention there is provided a crystalline form, Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol, which has an X-ray powder diffraction pattern with at least two specific peaks at about 2-theta=5.2° and 18.2°.

According to the present invention there is provided a crystalline form, Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol, which has an X-ray powder diffraction pattern with specific peaks at about 2-theta=5.2°, 18.2°, 18.7°, 14.5°, 23.4°, 25.3°, 25.9°, 16.6°, 21.5°, 15.8°.

According to the present invention there is provided crystalline form, Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol which has an X-ray powder diffraction pattern substantially the same as the X-ray powder diffraction pattern shown in FIG. 4.

According to the present invention there is provided crystalline form, Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol, which has an X-ray powder diffraction pattern with at least one specific peak at 2-theta=5.2° plus or minus 0.5° 2-theta.

According to the present invention there is provided a crystalline form, Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol, which has an X-ray powder diffraction pattern with at least one specific peak at 2-theta=18.2° plus or minus 0.5° 2-theta.

According to the present invention there is provided a crystalline form, Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol, which has an X-ray powder diffraction pattern with at least two specific peaks at 2-theta=5.2° and 18.2° wherein said values may be plus or minus 0.5° 2-theta.

According to the present invention there is provided a crystalline form, Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol, which has an X-ray powder diffraction pattern with specific peaks at 2-theta=5.2°, 18.2°, 18.7°, 14.5°, 23.4°, 25.3°, 25.9°, 16.6°, 21.5°, 15.8° wherein said values may be plus or minus 0.5° 2-theta.

According to the present invention there is provided a crystalline form, Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol, which has an X-ray powder diffraction pattern with at least one specific peak at 2-theta=5.2°.

According to the present invention there is provided a crystalline form, Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol, which has an X-ray powder diffraction pattern with at least one specific peak at 2-theta=18.2°.

According to the present invention there is provided a crystalline form, Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol, which has an X-ray powder diffraction pattern with at least two specific peaks at 2-theta=5.2° and 18.2°.

According to the present invention there is provided crystalline form, Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol, which has an X-ray powder diffraction pattern with specific peaks at 2-theta=5.2°, 18.2°, 18.7°, 14.5°, 23.4°, 25.3°, 25.9°, 16.6°, 21.5°, 15.8°.

According to the present invention there is provided crystalline form, Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol, which has an X-ray powder diffraction pattern as shown in FIG. 4.

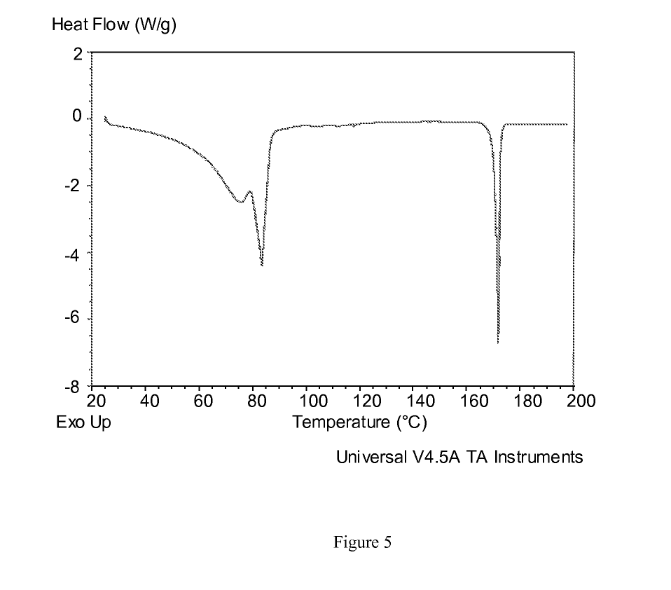

DSC analysis of Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol shows an initial endothermic event with an onset at 50.0° C. and a peak at 74.5° C. followed by a subsequent endothermic event with an onset of 80.6° C. and a peak at 83.8° C. This is then followed by a melt event with an onset at 170.6° C. and a peak at 171.8° (FIG. 5).

Thus DSC analysis shows Hydrate Form A of 2-[4-[4-[(5-fluoro-4-imidazo[1,2-a]pyridin-3-yl-pyrimidin-2-yl)amino]-3-methoxy-phenyl]piperazin-1-yl]ethanol is a high melting solid with an onset of melting at about 170.6° C. and a peak at 171.8°.

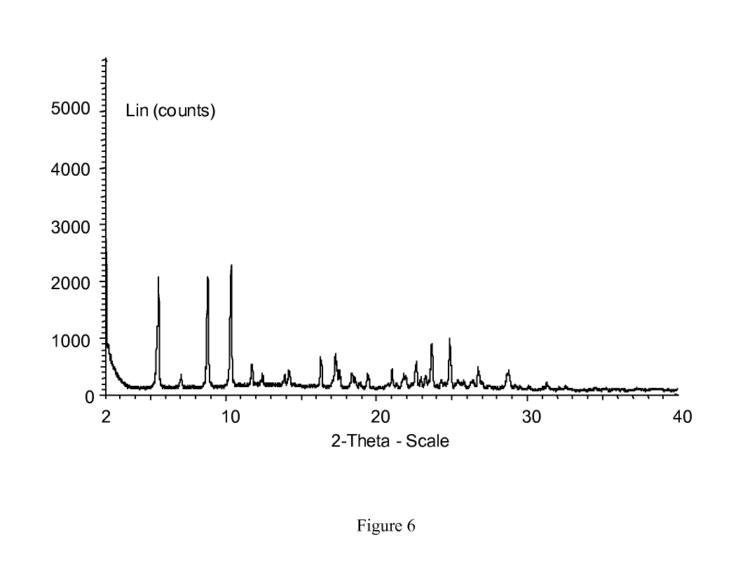

The present invention also provides a crystalline form of a 2-[4-[4-[(5-fluoro-4-imidazo[1,2-a]pyridin-3-yl-pyrimidin-2-yl)amino]-3-methoxy-phenyl]piperazin-1-yl]ethanol phosphate salt. The 2-[4-[4-[(5-fluoro-4-imidazo[1,2-a]pyridin-3-yl-pyrimidin-2-yl)amino]-3-methoxy-phenyl]piperazin-1-yl]ethanol phosphate salt is characterised in providing at least one of the following 2θ values measured using CuKa radiation: 10.3° and 5.5°. The 2-[4-[4-[(5-fluoro-4-imidazo[1,2-a]pyridin-3-yl-pyrimidin-2-yl)amino]-3-methoxy-phenyl]piperazin-1-yl]ethanol phosphate salt is characterised in providing an X-ray powder diffraction pattern, substantially as shown in FIG. 6. The ten most prominent peaks are shown in Table A:

TABLE 5
Ten most Prominent X-Ray Powder Diffraction peaks for 2-[4-[4-
[(5-fluoro-4-imidazo[1,2-a]pyridin-3-yl-pyrimidin-2-yl)amino]-3-
methoxy-phenyl]piperazin-1-yl]ethanol phosphate salt

| Angle 2- | | Relative |
|---|---|---|
| Theta (2θ) | Intensity % | Intensity |
| 10.310 | 100.0 | vs |
| 5.467 | 91.7 | vs |
| 8.761 | 91.2 | vs |
| 24.867 | 43.1 | vs |
| 23.665 | 37.2 | vs |
| 17.242 | 31.9 | vs |
| 22.622 | 26.0 | vs |
| 16.298 | 25.5 | vs |
| 11.716 | 23.3 | s |
| 26.754 | 20.9 | s |

vs = very strong
s = strong

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol phosphate salt which has an X-ray powder diffraction pattern with at least one specific peak at about 2-theta=10.3°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol phosphate salt which has an X-ray powder diffraction pattern with at least one specific peak at about 2-theta=5.5°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol phosphate salt, which has an X-ray powder diffraction pattern with at least two specific peaks at about 2-theta=10.3° and 5.5°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol phosphate salt, which has an X-ray powder diffraction pattern with specific peaks at about 2-theta=10.3°, 5.5°, 8.8°, 24.9°, 23.7°, 17.2°, 22.6°, 16.3°, 11.7°, 26.8°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol phosphate salt, which has an X-ray powder diffraction pattern substantially the same as the X-ray powder diffraction pattern shown in FIG. 6.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol phosphate salt, which has an X-ray powder diffraction pattern with at least one specific peak at 2-theta=10.3° plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol phosphate salt, which has an X-ray powder diffraction pattern with at least one specific peak at 2-theta=5.5° plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol phosphate salt, which has an X-ray powder diffraction pattern with at least two specific peaks at 2-theta=10.3° and 5.5° wherein said values may be plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol phosphate salt, which has an X-ray powder diffraction pattern with specific peaks at 2-theta=10.3°, 5.5°, 8.8°, 24.9°, 23.7°, 17.2°, 22.6°, 16.3°, 11.7°, 26.8° wherein said values may be plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol phosphate salt, which has an X-ray powder diffraction pattern with at least one specific peak at 2-theta=10.3°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol phosphate salt, which has an X-ray powder diffraction pattern with at least one specific peak at 2-theta=5.5°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol phosphate salt, which has an X-ray powder diffraction pattern with at least two specific peaks at 2-theta=10.3° and 5.5°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol phosphate salt, which has an X-ray powder diffraction pattern with specific peaks at 2-theta=10.3°, 5.5°, 8.8°, 24.9°, 23.7°, 17.2°, 22.6°, 16.3°, 11.7°, 26.8°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol phosphate salt, which has an X-ray powder diffraction pattern as shown in FIG. 6.

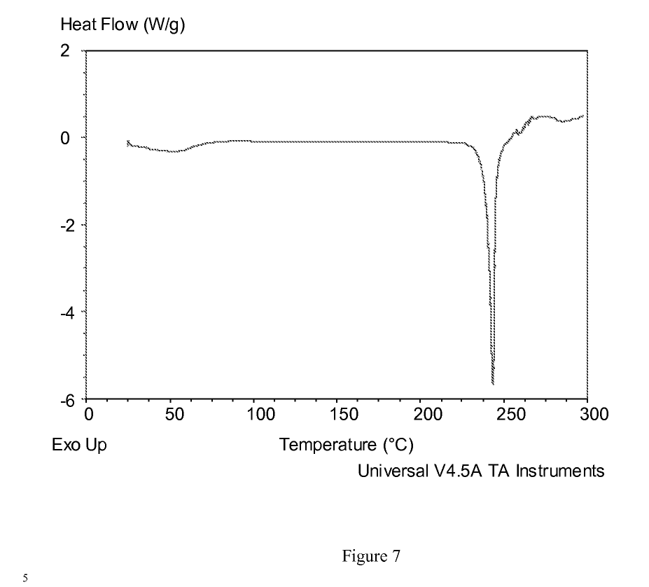

DSC analysis of the 2-[4-[4-[(5-fluoro-4-imidazo[1,2-a]pyridin-3-yl-pyrimidin-2-yl)amino]-3-methoxy-phenyl]piperazin-1-yl]ethanol phosphate salt shows an initial event with an onset at 29.8° C. and a peak at 54.0° C. followed by a subsequent melt with an onset of 239.5° C. and a peak at 243.4° C. (FIG. 7).

Thus DSC analysis shows 2-[4-[4-[(5-fluoro-4-imidazo[1,2-a]pyridin-3-yl-pyrimidin-2-yl)amino]-3-methoxy-phenyl]piperazin-1-yl]ethanol phosphate salt is a high melting solid with an onset of melting at about 239.5° C. and a peak at 243.4° C.

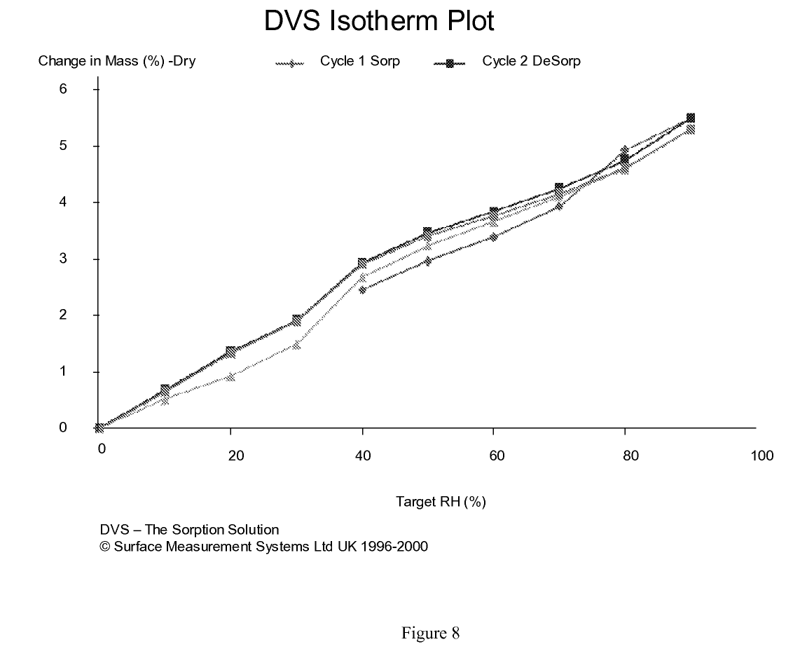

Dynamic vapour sorption (FIG. 8) in combination with XRPD and DSC suggest that the salt may be a channel hydrate.

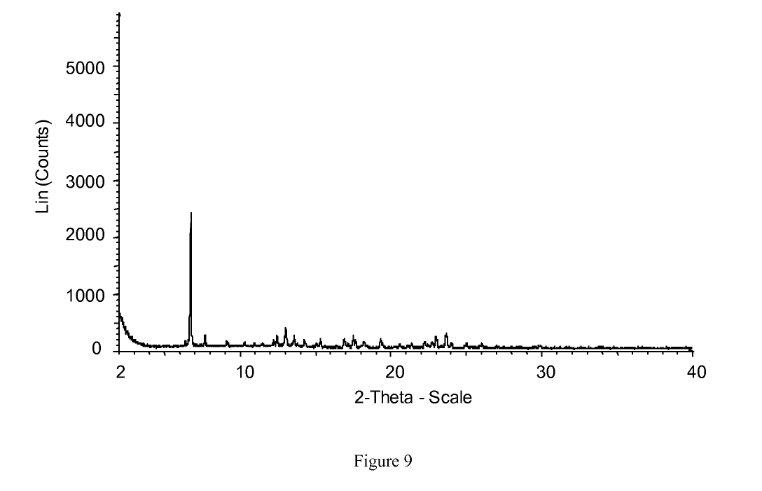

The present invention also provides a crystalline form of a 2-[4-[4-[(5-fluoro-4-imidazo[1,2-a]pyridin-3-yl-pyrimidin-2-yl)amino]-3-methoxy-phenyl]piperazin-1-yl]ethanol besylate salt. The 2-[4-[4-[(5-fluoro-4-imidazo[1,2-a]pyridin-3-yl-pyrimidin-2-yl)amino]-3-methoxy-phenyl]piperazin-1-yl]ethanol besylate salt is characterised in providing at least one of the following 20 values measured using CuKa radiation: 6.7° and 13.0°. The 2-[4-[4-[(5-fluoro-4-imidazo[1,2-a]pyridin-3-yl-pyrimidin-2-yl)amino]-3-methoxy-phenyl]piperazin-1-yl]ethanol besylate salt is characterised in providing an X-ray powder diffraction pattern, substantially as shown in FIG. 9. The ten most prominent peaks are shown in Table 6:

TABLE 6
Ten most Prominent X-Ray Powder Diffraction peaks for 2-[4-
[4-[(5-fluoro-4-imidazo[1,2-a]pyridin-3-yl-pyrimidin-2-yl)amino]-
3-methoxy-phenyl]piperazin-1-yl]ethanol besylate salt

| Angle 2- | | Relative |
|---|---|---|
| Theta (2θ) | Intensity % | Intensity |
| 6.703 | 100.0 | vs |
| 13.029 | 15.9 | s |
| 23.674 | 13.0 | s |
| 7.650 | 11.3 | s |
| 17.484 | 11.2 | s |
| 12.445 | 11.2 | s |
| 13.572 | 11.0 | s |
| 12.951 | 10.8 | s |
| 23.001 | 10.5 | s |
| 15.324 | 8.9 | m |

vs = very strong
s = strong
m = medium

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol besylate salt, which has an X-ray powder diffraction pattern with at least one specific peak at about 2-theta=6.7°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol besylate salt, which has an X-ray powder diffraction pattern with at least one specific peak at about 2-theta=13.0°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol besylate salt, which has an X-ray powder diffraction pattern with at least two specific peaks at about 2-theta=6.7° and 13.0°.

According to the present invention there is provided a crystalline form, besylate salt, which has an X-ray powder diffraction pattern with specific peaks at about 2-theta=6.7°, 13.0°, 23.7°, 7.7°, 17.5°, 12.5°, 13.6°, 13.0°, 23.0°, 15.3°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol besylate salt, which has an X-ray powder diffraction pattern substantially the same as the X-ray powder diffraction pattern shown in FIG. 9.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol besylate salt, which has an X-ray powder diffraction pattern with at least one specific peak at 2-theta=6.7° plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol besylate salt, which has an X-ray powder diffraction pattern with at least one specific peak at 2-theta=13.0° plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol besylate salt, which has an X-ray powder diffraction pattern with at least two specific peaks at 2-theta=6.7° and 13.0° wherein said values may be plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol besylate salt, which has an X-ray powder diffraction pattern with specific peaks at 2-theta=6.7°, 13.0°, 23.7°, 7.7°, 17.5°, 12.5°, 13.6°, 13.0°, 23.0°, 15.3° wherein said values may be plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol besylate salt, which has an X-ray powder diffraction pattern with at least one specific peak at 2-theta=6.7°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol besylate salt, which has an X-ray powder diffraction pattern with at least one specific peak at 2-theta=13.0°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol besylate salt, which has an X-ray powder diffraction pattern with at least two specific peaks at 2-theta=6.7° and 13.0°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol besylate salt, which has an X-ray powder diffraction pattern with specific peaks at 2-theta=6.7°, 13.0°, 23.7°, 7.7°, 17.5°, 12.5°, 13.6°, 13.0°, 23.0°, 15.3°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol besylate salt, which has an X-ray powder diffraction pattern as shown in FIG. 9.

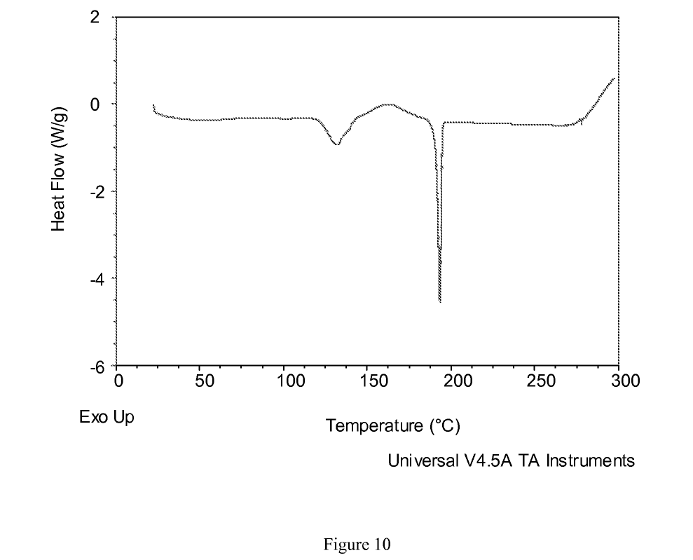

DSC analysis of 2-[4-[4-[(5-fluoro-4-imidazo[1,2-a]pyridin-3-yl-pyrimidin-2-yl)amino]-3-methoxy-phenyl]piperazin-1-yl]ethanol besylate salt, shows an initial event with an onset at 122.73° C. and a peak at 132.10° C. followed by a subsequent melt with an onset of 191.20° C. and a peak of 193.38° C. (FIG. 10).

Thus DSC analysis shows 2-[4-[4-[(5-fluoro-4-imidazo[1,2-a]pyridin-3-yl-pyrimidin-2-yl)amino]-3-methoxy-phenyl]piperazin-1-yl]ethanol besylate salt is a high melting solid with an onset of melting at about 191.20° C. and a peak of 193.38° C.

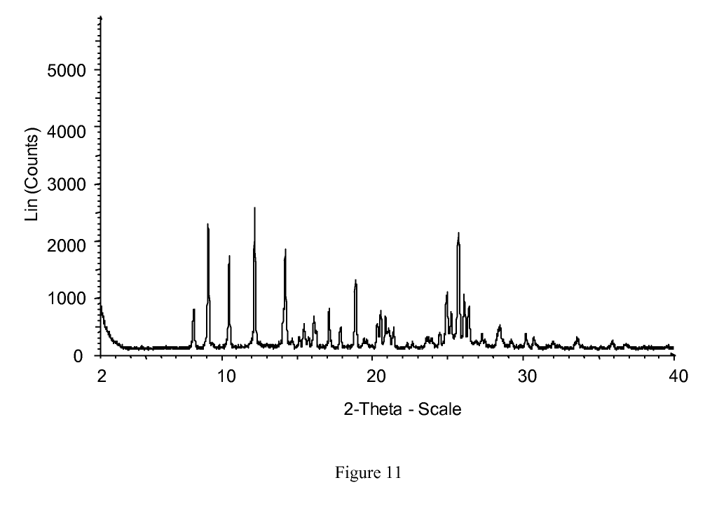

The present invention also provides a crystalline form of a 2-[4-[4-[(5-fluoro-4-imidazo[1,2-a]pyridin-3-yl-pyrimidin-2-yl)amino]-3-methoxy-phenyl]piperazin-1-yl]ethanol citrate salt. The 2-[4-[4-[(5-fluoro-4-imidazo[1,2-a]pyridin-3-yl-pyrimidin-2-yl)amino]-3-methoxy-phenyl]piperazin-1-yl]ethanol citrate salt is characterised in providing at least one of the following 2θ values measured using CuKa radiation: 12.2° and 9.1°. The 2-[4-[4-[(5-fluoro-4-imidazo[1,2-a]pyridin-3-yl-pyrimidin-2-yl)amino]-3-methoxy-phenyl]piperazin-1-yl]ethanol citrate salt is characterised in providing an X-ray powder diffraction pattern, substantially as shown in FIG. 11. The ten most prominent peaks are shown in Table 7:

TABLE 7
Ten most Prominent X-Ray Powder Diffraction peaks for 2-[4-[4-
[(5-fluoro-4-imidazo[1,2-a]pyridin-3-yl-pyrimidin-2-yl)amino]-3-
methoxy-phenyl]piperazin-1-yl]ethanol citrate salt

| Angle 2- | | Relative |
|---|---|---|
| Theta (2θ) | Intensity % | Intensity |
| 12.189 | 100.0 | vs |
| 9.098 | 88.8 | vs |
| 25.702 | 81.0 | vs |
| 14.209 | 71.3 | vs |
| 10.498 | 66.6 | vs |
| 18.881 | 50.6 | vs |
| 24.940 | 42.4 | vs |
| 26.089 | 40.3 | vs |
| 26.380 | 31.5 | vs |
| 17.106 | 31.1 | vs |

vs = very strong

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol citrate salt, which has an X-ray powder diffraction pattern with at least one specific peak at about 2-theta=12.2°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol citrate salt, which has an X-ray powder diffraction pattern with at least one specific peak at about 2-theta=9.1°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol citrate salt, which has an X-ray powder diffraction pattern with at least two specific peaks at about 2-theta=12.2° and 9.1°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol citrate salt, which has an X-ray powder diffraction pattern with specific peaks at about 2-theta=12.2°, 9.1°, 25.7°, 14.2°, 10.5°, 18.9°, 25.0°, 26.1°, 26.4°, 17.1°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol citrate salt, which has an X-ray powder diffraction pattern substantially the same as the X-ray powder diffraction pattern shown in FIG. 11.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol citrate salt, which has an X-ray powder diffraction pattern with at least one specific peak at 2-theta=12.2° plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol citrate salt, which has an X-ray powder diffraction pattern with at least one specific peak at 2-theta=9.1° plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol citrate salt, which has an X-ray powder diffraction pattern with at least two specific peaks at 2-theta=12.2° and 9.1° wherein said values may be plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol citrate salt, which has an X-ray powder diffraction pattern with specific peaks at 2-theta=12.2°, 9.1°, 25.7°, 14.2°, 10.5°, 18.9°, 25.0°, 26.1°, 26.4°, 17.1° wherein said values may be plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol citrate salt, which has an X-ray powder diffraction pattern with at least one specific peak at 2-theta=12.2°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol citrate salt, which has an X-ray powder diffraction pattern with at least one specific peak at 2-theta=9.1°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol citrate salt, which has an X-ray powder diffraction pattern with at least two specific peaks at 2-theta=12.2° and 9.1°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol citrate salt, which has an X-ray powder diffraction pattern with specific peaks at 2-theta=12.2°, 9.1°, 25.7°, 14.2°, 10.5°, 18.9°, 25.0°, 26.1°, 26.4°, 17.1°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol citrate salt, which has an X-ray powder diffraction pattern as shown in FIG. 11.

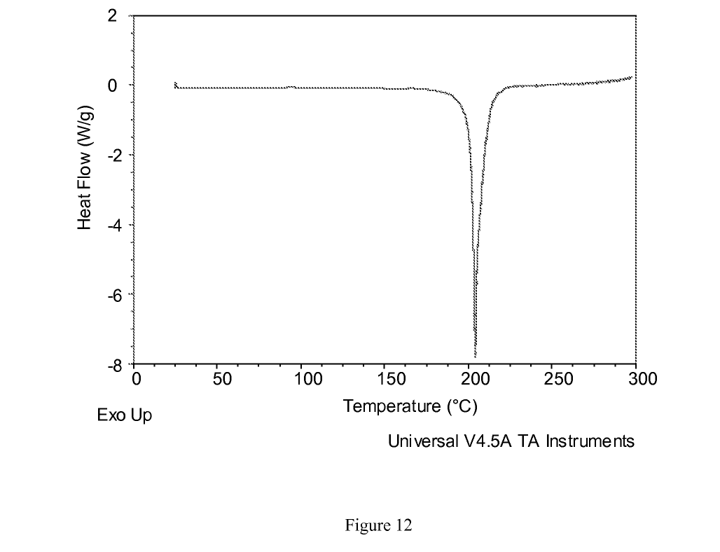

DSC analysis of 2-[4-[4-[(5-fluoro-4-imidazo[1,2-a]pyridin-3-yl-pyrimidin-2-yl)amino]-3-methoxy-phenyl]piperazin-1-yl]ethanol citrate salt shows melt with an onset of 202.00° C. and a peak at 204.14° C. (FIG. 12).

Thus DSC analysis shows 2-[4-[4-[(5-fluoro-4-imidazo[1,2-a]pyridin-3-yl-pyrimidin-2-yl)amino]-3-methoxy-phenyl]piperazin-1-yl]ethanol citrate salt is a high melting solid with an onset of melting at about 202.00° C. and a peak at 204.14° C.

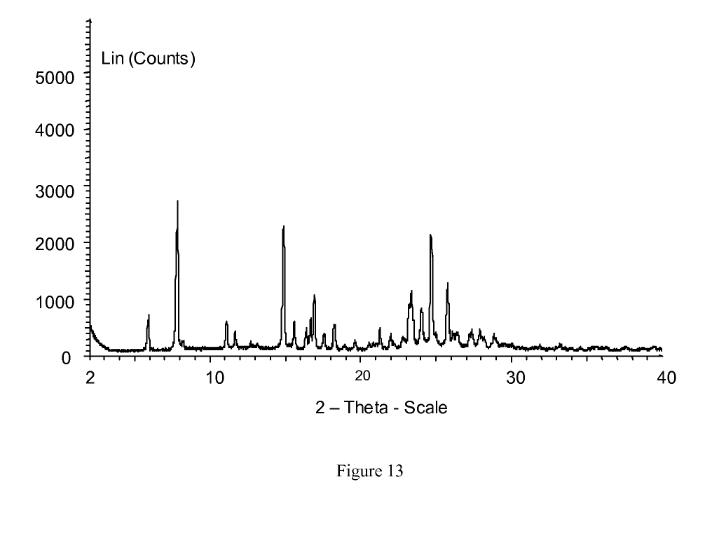

The present invention also provides a crystalline form of a 2-[4-[4-[(5-fluoro-4-imidazo[1,2-a]pyridin-3-yl-pyrimidin-2-yl)amino]-3-methoxy-phenyl]piperazin-1-yl]ethanol sulphate salt. The 2-[4-[4-[(5-fluoro-4-imidazo[1,2-a]pyridin-3-yl-pyrimidin-2-yl)amino]-3-methoxy-phenyl]piperazin-1-yl]ethanol sulphate salt is characterised in providing at least one of the following 2θ values measured using CuKa radiation: 7.8° and 14.9°. The 2-[4-[4-[(5-fluoro-4-imidazo[1,2-a]pyridin-3-yl-pyrimidin-2-yl)amino]-3-methoxy-phenyl]piperazin-1-yl]ethanol sulphate salt is characterised in providing an X-ray powder diffraction pattern, substantially as shown in FIG. 13. The ten most prominent peaks are shown in Table 8:

TABLE 8
Ten most Prominent X-Ray Powder Diffraction peaks for 2-[4-[4-
[(5-fluoro-4-imidazo[1,2-a]pyridin-3-yl-pyrimidin-2-yl)amino]-3-
methoxy-phenyl]piperazin-1-yl]ethanol sulphate salt

| Angle 2- | | Relative |
|---|---|---|
| Theta (2θ) | Intensity % | Intensity |
| 7.785 | 100.0 | vs |
| 14.858 | 83.6 | vs |
| 24.673 | 78.1 | vs |
| 25.753 | 46.9 | vs |
| 23.370 | 41.4 | vs |
| 16.895 | 39.1 | vs |
| 23.363 | 33.1 | vs |
| 24.017 | 30.8 | vs |
| 5.870 | 26.4 | vs |
| 16.648 | 24.7 | vs |

vs = very strong

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol sulphate salt, which has an X-ray powder diffraction pattern with at least one specific peak at about 2-theta=7.8°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol sulphate salt, which has an X-ray powder diffraction pattern with at least one specific peak at about 2-theta=14.9°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol sulphate salt, which has an X-ray powder diffraction pattern with at least two specific peaks at about 2-theta=7.8° and 14.9°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol sulphate salt, which has an X-ray powder diffraction pattern with specific peaks at about 2-theta=7.8°, 14.9°, 24.7°, 25.8°, 23.4°, 16.9°, 23.4°, 24.0°, 5.9°, 16.6°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol sulphate salt, which has an X-ray powder diffraction pattern substantially the same as the X-ray powder diffraction pattern shown in FIG. 13.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol sulphate salt, which has an X-ray powder diffraction pattern with at least one specific peak at 2-theta=7.8° plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol sulphate salt, which has an X-ray powder diffraction pattern with at least one specific peak at 2-theta=14.9° plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol sulphate salt, which has an X-ray powder diffraction pattern with at least two specific peaks at 2-theta=7.8° and 14.9° wherein said values may be plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol sulphate salt, which has an X-ray powder diffraction pattern with specific peaks at 2-theta=7.8°, 14.9°, 24.7°, 25.8°, 23.4°, 16.9°, 23.4°, 24.0°, 5.9°, 16.6° wherein said values may be plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol sulphate salt, which has an X-ray powder diffraction pattern with at least one specific peak at 2-theta=7.8°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol sulphate salt, which has an X-ray powder diffraction pattern with at least one specific peak at 2-theta=14.9°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol sulphate salt, which has an X-ray powder diffraction pattern with at least two specific peaks at 2-theta=7.8° and 14.9°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol sulphate salt, which has an X-ray powder diffraction pattern with specific peaks at 2-theta=7.8°, 14.9°, 24.7°, 25.8°, 23.4°, 16.9°, 23.4°, 24.0°, 5.9°, 16.6°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol sulphate salt, which has an X-ray powder diffraction pattern as shown in FIG. 13.

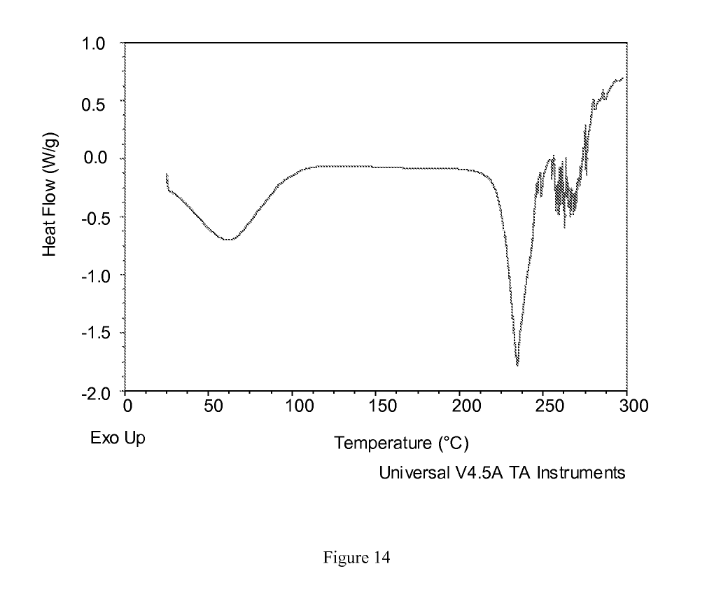

DSC analysis of the 2-[4-[4-[(5-fluoro-4-imidazo[1,2-a]pyridin-3-yl-pyrimidin-2-yl)amino]-3-methoxy-phenyl]piperazin-1-yl]ethanol sulphate salt, shows an initial event with an onset at 33.69° C. and a peak at 62.99° C. followed by a subsequent melt with an onset of 222.89° C. with a peak at 233.77° C. (FIG. 14).

Thus DSC analysis shows 2-[4-[4-[(5-fluoro-4-imidazo[1,2-a]pyridin-3-yl-pyrimidin-2-yl)amino]-3-methoxy-phenyl]piperazin-1-yl]ethanol sulphate salt is a high melting solid with an onset of melting at about 222.89° C. with a peak at 233.77° C.

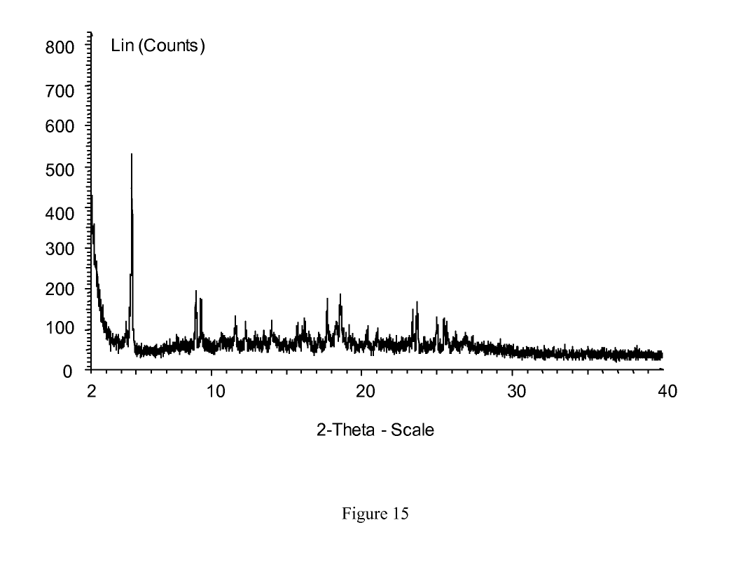

The present invention also provides a crystalline form of a 2-[4-[4-[(5-fluoro-4-imidazo[1,2-a]pyridin-3-yl-pyrimidin-2-yl)amino]-3-methoxy-phenyl]piperazin-1-yl]ethanol tosylate salt. The 2-[4-[4-[(5-fluoro-4-imidazo[1,2-a]pyridin-3-yl-pyrimidin-2-yl)amino]-3-methoxy-phenyl]piperazin-1-yl]ethanol tosylate salt is characterised in providing at least one of the following 2θ values measured using CuKa radiation: 4.7° and 9.0°. The 2-[4-[4-[(5-fluoro-4-imidazo[1,2-a]pyridin-3-yl-pyrimidin-2-yl)amino]-3-methoxy-phenyl]piperazin-1-yl]ethanol tosylate salt is characterised in providing an X-ray powder diffraction pattern, substantially as shown in FIG. 15. The ten most prominent peaks are shown in Table 9:

TABLE 9
Ten most Prominent X-Ray Powder Diffraction peaks for 2-[4-[4-[(5-
fluoro-4-imidazo[1,2-a]pyridin-3-yl-pyrimidin-2-yl)amino]-3-methoxy-
phenyl]piperazin-1-yl]ethanol tosylate salt

| Angle 2- | | Relative |
|---|---|---|
| Theta (2θ) | Intensity % | Intensity |
| 4.684 | 100.0 | vs |
| 8.950 | 36.2 | vs |
| 18.567 | 35.0 | vs |
| 17.687 | 32.6 | vs |
| 9.293 | 32.2 | vs |
| 23.686 | 31.6 | vs |
| 23.363 | 27.9 | vs |
| 11.588 | 24.9 | s |
| 24.979 | 24.3 | s |
| 16.196 | 23.7 | s |

vs = very strong
s = strong

According to the present invention there is provided a crystalline form, the 2-[4-[4-[(5-fluoro-4-imidazo[1,2-a]pyridin-3-yl-pyrimidin-2-yl)amino]-3-methoxy-phenyl]piperazin-1-yl]ethanol tosylate salt which has an X-ray powder diffraction pattern with at least one specific peak at about 2-theta=4.7°.

According to the present invention there is provided a crystalline form, the 2-[4-[4-[(5-fluoro-4-imidazo[1,2-a]pyridin-3-yl-pyrimidin-2-yl)amino]-3-methoxy-phenyl]piperazin-1-yl]ethanol tosylate salt which has an X-ray powder diffraction pattern with at least one specific peak at about 2-theta=9.0°.

According to the present invention there is provided a crystalline form, the 2-[4-[4-[(5-fluoro-4-imidazo[1,2-a]pyridin-3-yl-pyrimidin-2-yl)amino]-3-methoxy-phenyl]piperazin-1-yl]ethanol tosylate salt, which has an X-ray powder diffraction pattern with at least two specific peaks at about 2-theta=4.7° and 9.0°.

According to the present invention there is provided a crystalline form, the 2-[4-[4-[(5-fluoro-4-imidazo[1,2-a]pyridin-3-yl-pyrimidin-2-yl)amino]-3-methoxy-phenyl]piperazin-1-yl]ethanol tosylate salt, which has an X-ray powder diffraction pattern with specific peaks at about 2-theta=4.7°, 9.0°, 18.6°, 17.7°, 9.3°, 23.7°, 23.4°, 11.6°, 25.0°, 16.2°.

According to the present invention there is provided crystalline form, the 2-[4-[4-[(5-fluoro-4-imidazo[1,2-a]pyridin-3-yl-pyrimidin-2-yl)amino]-3-methoxy-phenyl]piperazin-1-yl]ethanol tosylate salt, which has an X-ray powder diffraction pattern substantially the same as the X-ray powder diffraction pattern shown in FIG. 15.

According to the present invention there is provided crystalline form, the 2-[4-[4-[(5-fluoro-4-imidazo[1,2-a]pyridin-3-yl-pyrimidin-2-yl)amino]-3-methoxy-phenyl]piperazin-1-yl]ethanol tosylate salt, which has an X-ray powder diffraction pattern with at least one specific peak at 2-theta=4.7° plus or minus 0.5° 2-theta.

According to the present invention there is provided a crystalline form, the 2-[4-[4-[(5-fluoro-4-imidazo[1,2-a]pyridin-3-yl-pyrimidin-2-yl)amino]-3-methoxy-phenyl]piperazin-1-yl]ethanol tosylate salt, which has an X-ray powder diffraction pattern with at least one specific peak at 2-theta=9.0° plus or minus 0.5° 2-theta.

According to the present invention there is provided a crystalline form, the 2-[4-[4-[(5-fluoro-4-imidazo[1,2-a]pyridin-3-yl-pyrimidin-2-yl)amino]-3-methoxy-phenyl]piperazin-1-yl]ethanol tosylate salt, which has an X-ray powder diffraction pattern with at least two specific peaks at 2-theta=4.7° and 9.0° wherein said values may be plus or minus 0.5° 2-theta.

According to the present invention there is provided a crystalline form, the 2-[4-[4-[(5-fluoro-4-imidazo[1,2-a]pyridin-3-yl-pyrimidin-2-yl)amino]-3-methoxy-phenyl]piperazin-1-yl]ethanol tosylate salt, which has an X-ray powder diffraction pattern with specific peaks at 2-theta=4.7°, 9.0°, 18.6°, 17.7°, 9.3°, 23.7°, 23.4°, 11.6°, 25.0°, 16.2°, wherein said values may be plus or minus 0.5° 2-theta.

According to the present invention there is provided a crystalline form, the 2-[4-[4-[(5-fluoro-4-imidazo[1,2-a]pyridin-3-yl-pyrimidin-2-yl)amino]-3-methoxy-phenyl]piperazin-1-yl]ethanol tosylate salt, which has an X-ray powder diffraction pattern with at least one specific peak at 2-theta=4.7°.

According to the present invention there is provided a crystalline form, the 2-[4-[4-[(5-fluoro-4-imidazo[1,2-a]pyridin-3-yl-pyrimidin-2-yl)amino]-3-methoxy-phenyl]piperazin-1-yl]ethanol tosylate salt, which has an X-ray powder diffraction pattern with at least one specific peak at 2-theta=9.0°.

According to the present invention there is provided a crystalline form, the 2-[4-[4-[(5-fluoro-4-imidazo[1,2-a]pyridin-3-yl-pyrimidin-2-yl)amino]-3-methoxy-phenyl]piperazin-1-yl]ethanol tosylate salt, which has an X-ray powder diffraction pattern with at least two specific peaks at 2-theta=4.7° and 9.0°.

According to the present invention there is provided crystalline form, the 2-[4-[4-[(5-fluoro-4-imidazo[1,2-a]pyridin-3-yl-pyrimidin-2-yl)amino]-3-methoxy-phenyl]piperazin-1-yl]ethanol tosylate salt, which has an X-ray powder diffraction pattern with specific peaks at 2-theta=4.7°, 9.0°, 18.6°, 17.7°, 9.3°, 23.7°, 23.4°, 11.6°, 25.0°, 16.2°.

According to the present invention there is provided crystalline form, the tosylate salt, which has an X-ray powder diffraction pattern as shown in FIG. 15.

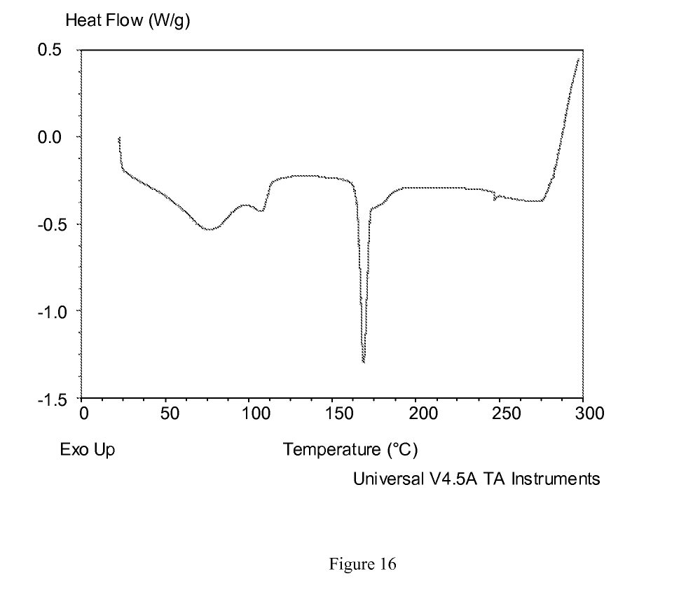

DSC analysis of 2-[4-[4-[(5-fluoro-4-imidazo[1,2-a]pyridin-3-yl-pyrimidin-2-yl)amino]-3-methoxy-phenyl]piperazin-1-yl]ethanol tosylate salt shows an initial event with an onset at 40.59° C. and a peak at 73.0° C. followed by a subsequent event with an onset at 100.84° C. followed by a peak at 108.78° C. The melt occurs after these events and has an onset of 165.00° C. and a peak at 168.57° C. (FIG. 16).

Thus DSC analysis shows 2-[4-[4-[(5-fluoro-4-imidazo[1,2-a]pyridin-3-yl-pyrimidin-2-yl)amino]-3-methoxy-phenyl]piperazin-1-yl]ethanol tosylate salt is a high melting solid with an onset of melting at about 165.00° C. and a peak at 168.57° C.

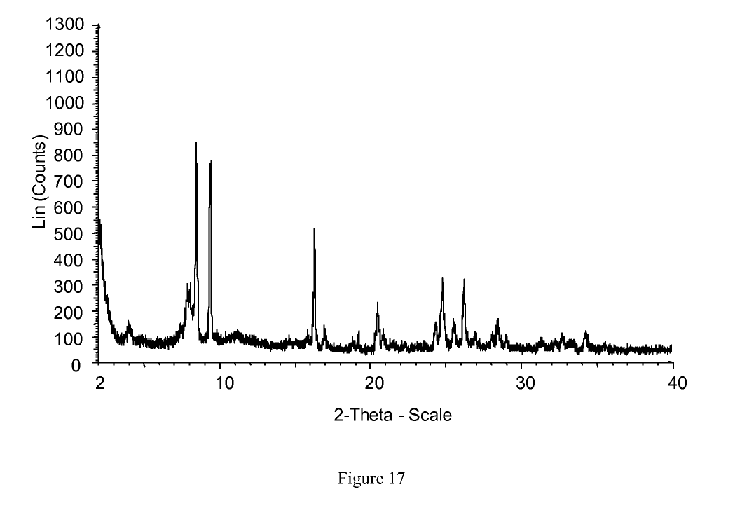

The present invention also provides a crystalline form of a 2-[4-[4-[(5-fluoro-4-imidazo[1,2-a]pyridin-3-yl-pyrimidin-2-yl)amino]-3-methoxy-phenyl]piperazin-1-yl]ethanol hydrochloride salt. The 2-[4-[4-[(5-fluoro-4-imidazo[1,2-a]pyridin-3-yl-pyrimidin-2-yl)amino]-3-methoxy-phenyl]piperazin-1-yl]ethanol hydrochloride salt is characterised in providing at least one of the following 2θ values measured using CuKa radiation: 8.4° and 9.4°. The 2-[4-[4-[(5-fluoro-4-imidazo[1,2-a]pyridin-3-yl-pyrimidin-2-yl)amino]-3-methoxy-phenyl]piperazin-1-yl]ethanol hydrochloride salt is characterised in providing an X-ray powder diffraction pattern, substantially as shown in FIG. 17. The ten most prominent peaks are shown in Table 10:

TABLE 10
Ten most Prominent X-Ray Powder Diffraction peaks for
2-[4-[4-[(5-fluoro-4-imidazo[1,2-a]pyridin-3-yl-pyrimidin-2-yl)amino]-
3-methoxy-phenyl]piperazin-1-yl]ethanol HCl salt

| Angle 2- | | Relative |
|---|---|---|
| Theta (2θ) | Intensity % | Intensity |
| 8.430 | 100.0 | vs |
| 9.372 | 91.5 | vs |
| 16.280 | 60.2 | vs |
| 26.182 | 37.7 | vs |
| 24.782 | 36.9 | vs |
| 8.042 | 34.9 | vs |
| 7.830 | 33.3 | vs |
| 20.503 | 23.6 | s |
| 28.430 | 19.9 | s |
| 25.509 | 18.4 | s |

vs = very strong
s = strong

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol HCl salt, which has an X-ray powder diffraction pattern with at least one specific peak at about 2-theta=8.4°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol HCl salt, which has an X-ray powder diffraction pattern with at least one specific peak at about 2-theta=9.4°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol HCl salt, which has an X-ray powder diffraction pattern with at least two specific peaks at about 2-theta=8.4° and 9.4°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol HCl salt, which has an X-ray powder diffraction pattern with specific peaks at about 2-theta=8.4°, 9.4°, 16.3°, 26.2°, 24.8°, 8.0°, 7.8°, 20.5°, 28.4°, 25.5°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol HCl salt, which has an X-ray powder diffraction pattern substantially the same as the X-ray powder diffraction pattern shown in FIG. 17.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol HCl salt, which has an X-ray powder diffraction pattern with at least one specific peak at 2-theta=8.4° plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol HCl salt, which has an X-ray powder diffraction pattern with at least one specific peak at 2-theta=9.4° plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol HCl salt, which has an X-ray powder diffraction pattern with at least two specific peaks at 2-theta=8.4° and 9.4° wherein said values may be plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol HCl salt, which has an X-ray powder diffraction pattern with specific peaks at 2-theta=8.4°, 9.4°, 16.3°, 26.2°, 24.8°, 8.0°, 7.8°, 20.5°, 28.4°, 25.5° wherein said values may be plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol HCl salt, which has an X-ray powder diffraction pattern with at least one specific peak at 2-theta=8.4°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol HCl salt, which has an X-ray powder diffraction pattern with at least one specific peak at 2-theta=9.4°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol HCl salt, which has an X-ray powder diffraction pattern with at least two specific peaks at 2-theta=8.4° and 9.4°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol HCl salt, which has an X-ray powder diffraction pattern with specific peaks at 2-theta=8.4°, 9.4°, 16.3°, 26.2°, 24.8°, 8.0°, 7.8°, 20.5°, 28.4°, 25.5°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol HCl salt, which has an X-ray powder diffraction pattern as shown in FIG. 17.

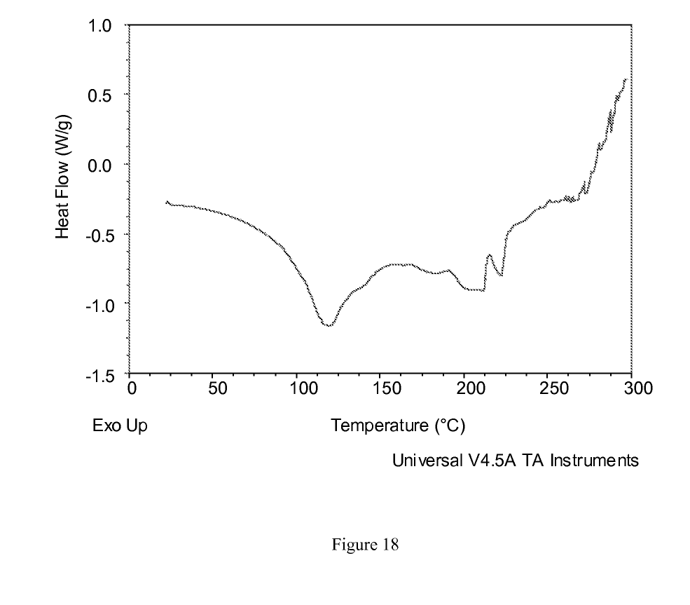

DSC analysis of 2-[4-[4-[(5-fluoro-4-imidazo[1,2-a]pyridin-3-yl-pyrimidin-2-yl)amino]-3-methoxy-phenyl]piperazin-1-yl]ethanol hydrochloride salt shows an initial event with an onset at 92.42° C. and a peak at 116.20° C. followed by a subsequent melt with an onset of 19.381° C. and a peak at 211.74° C. (FIG. 18).

Thus DSC analysis shows 2-[4-[4-[(5-fluoro-4-imidazo[1,2-a]pyridin-3-yl-pyrimidin-2-yl)amino]-3-methoxy-phenyl]piperazin-1-yl]ethanol hydrochloride salt is a high melting solid with an onset of melting at about 19.381° C. and a peak at 211.74° C.

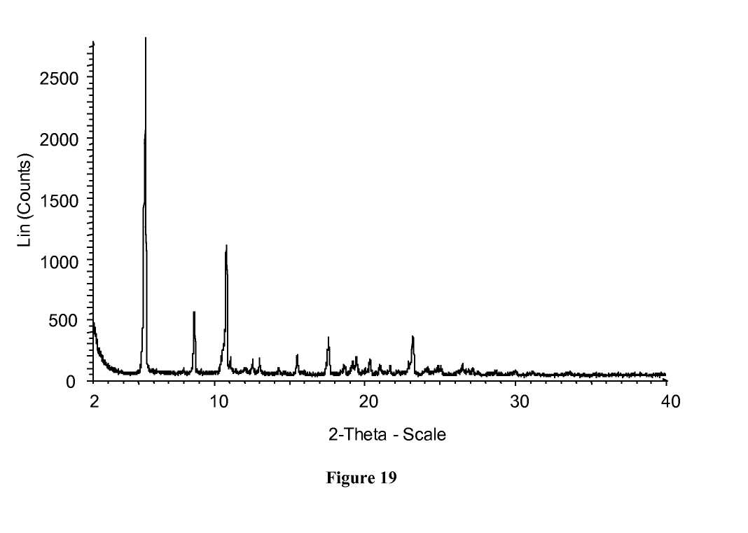

The present invention also provides a crystalline form of a 2-[4-[4-[(5-fluoro-4-imidazo[1,2-a]pyridin-3-yl-pyrimidin-2-yl)amino]-3-methoxy-phenyl]piperazin-1-yl]ethanol fumarate salt. The 2-[4-[4-[(5-fluoro-4-imidazo[1,2-a]pyridin-3-yl-pyrimidin-2-yl)amino]-3-methoxy-phenyl]piperazin-1-yl]ethanol fumerate salt is characterised in providing at least one of the following 2θ values measured using CuKa radiation: 5.4° and 10.8°. The 2-[4-[4-[(5-fluoro-4-imidazo[1,2-a]pyridin-3-yl-pyrimidin-2-yl)amino]-3-methoxy-phenyl]piperazin-1-yl]ethanol fumerate salt is characterised in providing an X-ray powder diffraction pattern, substantially as shown in FIG. 19. The ten most prominent peaks are shown in Table 11:

TABLE 11
Ten most Prominent X-Ray Powder Diffraction peaks for
2-[4-[4-[(5-fluoro-4-imidazo[1,2-a]pyridin-3-yl-pyrimidin-2-yl)amino]-
3-methoxy-phenyl]piperazin-1-yl]ethanol fumerate salt

| Angle 2- | | Relative |
|---|---|---|
| Theta (2θ) | Intensity % | Intensity |
| 5.407 | 100.0 | vs |
| 10.804 | 39.1 | vs |
| 8.678 | 19.9 | s |
| 23.202 | 12.9 | s |
| 17.587 | 12.0 | s |
| 10.618 | 11.2 | s |
| 15.503 | 7.2 | m |
| 19.452 | 6.5 | m |
| 11.086 | 6.4 | m |
| 13.010 | 6.2 | m |

vs = very strong
s = strong
m = medium

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol fumerate salt, which has an X-ray powder diffraction pattern with at least one specific peak at about 2-theta=5.4°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol fumerate salt, which has an X-ray powder diffraction pattern with at least one specific peak at about 2-theta=10.8°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol fumerate salt, which has an X-ray powder diffraction pattern with at least two specific peaks at about 2-theta=5.4° and 10.8°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol fumerate salt, which has an X-ray powder diffraction pattern with specific peaks at about 2-theta=5.4°, 10.8°, 8.7°, 23.2°, 17.6°, 10.6°, 15.5°, 19.5°, 11.1°, 13.0°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol fumerate salt, which has an X-ray powder diffraction pattern substantially the same as the X-ray powder diffraction pattern shown in FIG. 19.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol fumerate salt, which has an X-ray powder diffraction pattern with at least one specific peak at 2-theta=5.4° plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol fumerate salt, which has an X-ray powder diffraction pattern with at least one specific peak at 2-theta=10.8° plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol fumerate salt, which has an X-ray powder diffraction pattern with at least two specific peaks at 2-theta=5.4° and 10.8° wherein said values may be plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol fumerate salt, which has an X-ray powder diffraction pattern with specific peaks at 2-theta=10.8°, 8.7°, 23.2°, 17.6°, 10.6°, 15.5°, 19.5°, 11.1°, 13.0° wherein said values may be plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol fumerate salt, which has an X-ray powder diffraction pattern with at least one specific peak at 2-theta=5.4°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol fumerate salt, which has an X-ray powder diffraction pattern with at least one specific peak at 2-theta=10.8°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol fumerate salt, which has an X-ray powder diffraction pattern with at least two specific peaks at 2-theta=5.4° and 10.8°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol fumerate salt, which has an X-ray powder diffraction pattern with specific peaks at 2-theta=10.8°, 8.7°, 23.2°, 17.6°, 10.6°, 15.5°, 19.5°, 11.1°, 13.0°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol fumerate salt, which has an X-ray powder diffraction pattern as shown in FIG. 19.

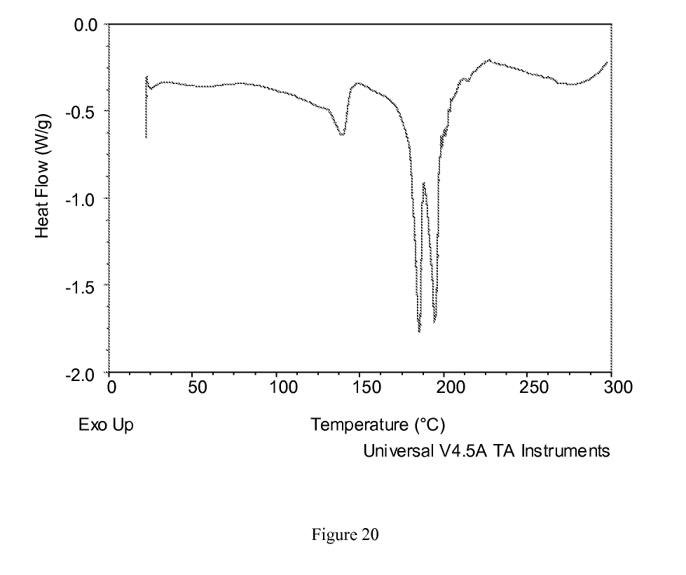

DSC analysis of 2-[4-[4-[(5-fluoro-4-imidazo[1,2-a]pyridin-3-yl-pyrimidin-2-yl)amino]-3-methoxy-phenyl]piperazin-1-yl]ethanol fumerate salt shows an initial event with an onset at 127.60° C. and a peak at 139.60° C. followed by a subsequent event with an onset at 180.03° C. and a peak at 185.16° C. this is then followed by a melt with an onset of 191.42° C. and a peak at 195.27° C. (FIG. 20).

Thus DSC analysis shows 2-[4-[4-[(5-fluoro-4-imidazo[1,2-a]pyridin-3-yl-pyrimidin-2-yl)amino]-3-methoxy-phenyl]piperazin-1-yl]ethanol fumerate salt is a high melting solid with an onset of melting at about 191.42° C. and a peak at 195.27° C.

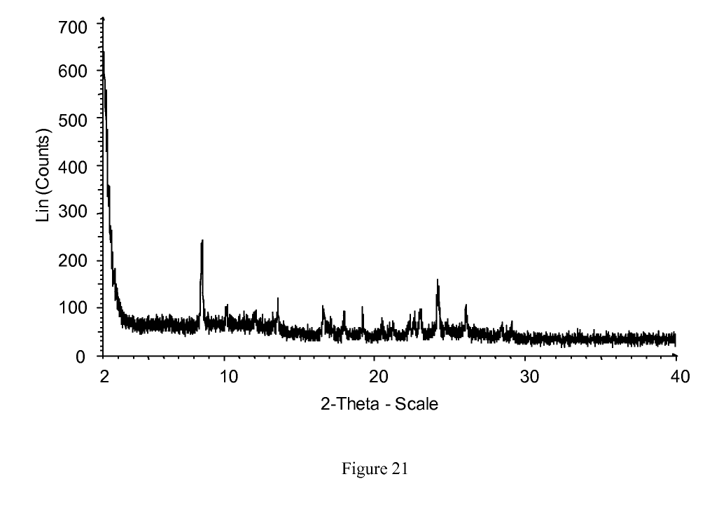

The present invention also provides a crystalline form of a 2-[4-[4-[(5-fluoro-4-imidazo[1,2-a]pyridin-3-yl-pyrimidin-2-yl)amino]-3-methoxy-phenyl]piperazin-1-yl]ethanol glycolate salt. The 2-[4-[4-[(5-fluoro-4-imidazo[1,2-a]pyridin-3-yl-pyrimidin-2-yl)amino]-3-methoxy-phenyl]piperazin-1-yl]ethanol glycolate salt is characterised in providing at least one of the following 2θ values measured using CuKa radiation: 8.5° and 24.2°. The 2-[4-[4-[(5-fluoro-4-imidazo[1,2-a]pyridin-3-yl-pyrimidin-2-yl)amino]-3-methoxy-phenyl]piperazin-1-yl]ethanol glycolate salt is characterised in providing an X-ray powder diffraction pattern, substantially as shown in FIG. 21. The ten most prominent peaks are shown in Table 12:

TABLE 12
Ten most Prominent X-Ray Powder Diffraction peaks for
2-[4-[4-[(5-fluoro-4-imidazo[1,2-a]pyridin-3-yl-pyrimidin-2-yl)amino]-
3-methoxy-phenyl]piperazin-1-yl]ethanol glycolate salt

| Angle 2- | | Relative |
|---|---|---|
| Theta (2θ) | Intensity % | Intensity |
| 8.516 | 100.0 | vs |
| 24.189 | 63.2 | vs |
| 13.563 | 51.3 | vs |
| 10.164 | 44.9 | vs |
| 23.028 | 41.9 | vs |
| 17.945 | 39.3 | vs |
| 26.069 | 37.6 | vs |
| 16.599 | 37.2 | vs |
| 26.103 | 36.3 | vs |
| 22.645 | 35.9 | vs |

vs = very strong

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol glycolate salt which has an X-ray powder diffraction pattern with at least one specific peak at about 2-theta=8.5°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol glycolate salt which has an X-ray powder diffraction pattern with at least one specific peak at about 2-theta=24.2°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol glycolate salt which has an X-ray powder diffraction pattern with at least two specific peaks at about 2-theta=8.5° and 24.2°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol glycolate salt, which has an X-ray powder diffraction pattern with specific peaks at about 2-theta=8.5°, 24.2°, 13.6°, 10.2°, 23.0°, 17.9°, 26.1°, 16.6°, 26.1°, 22.6°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol glycolate salt which has an X-ray powder diffraction pattern substantially the same as the X-ray powder diffraction pattern shown in FIG. 21.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol glycolate salt, which has an X-ray powder diffraction pattern with at least one specific peak at 2-theta=8.5° plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol glycolate salt, which has an X-ray powder diffraction pattern with at least one specific peak at 2-theta=24.2° plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol glycolate salt, which has an X-ray powder diffraction pattern with at least two specific peaks at 2-theta=8.5° and 24.2° wherein said values may be plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol glycolate salt, which has an X-ray powder diffraction pattern with specific peaks at 2-theta=8.5°, 24.2°, 13.6°, 10.2°, 23.0°, 17.9°, 26.1°, 16.6°, 26.1°, 22.6° wherein said values may be plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol glycolate salt, which has an X-ray powder diffraction pattern with at least one specific peak at 2-theta=8.5°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol glycolate salt, which has an X-ray powder diffraction pattern with at least one specific peak at 2-theta=24.2°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol glycolate salt, which has an X-ray powder diffraction pattern with at least two specific peaks at 2-theta=8.5° and 24.2°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol glycolate salt, which has an X-ray powder diffraction pattern with specific peaks at 2-theta=8.5°, 24.2°, 13.6°, 10.2°, 23.0°, 17.9°, 26.1°, 16.6°, 26.1°, 22.6°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol glycolate salt, which has an X-ray powder diffraction pattern as shown in FIG. 21.

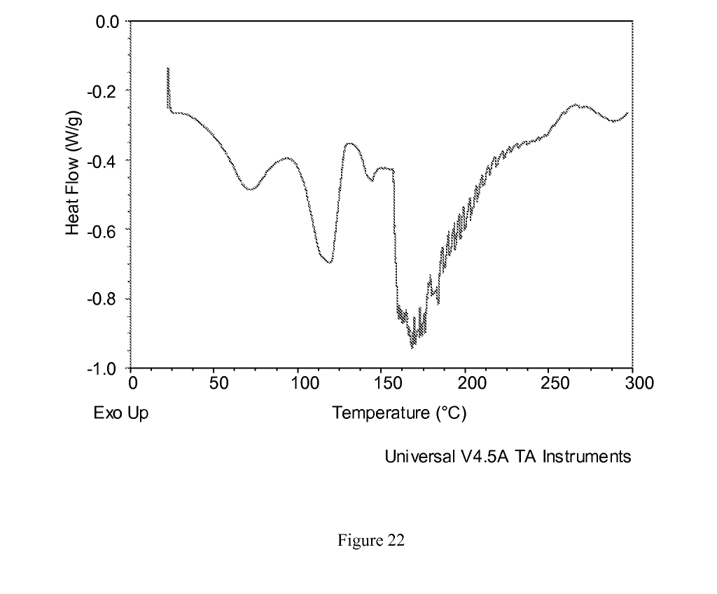

DSC analysis of 2-[4-[4-[(5-fluoro-4-imidazo[1,2-a]pyridin-3-yl-pyrimidin-2-yl)amino]-3-methoxy-phenyl]piperazin-1-yl]ethanol glycolate salt shows an initial event with an onset at 48.5° C. and a peak at 68.8° C. followed by a second event with an onset at 102.1° C. and a peak at 119.8° C. A further event occurs before the melt with an onset at 136.7 and a peak at 141.5° C. with a subsequent melt with an onset of 157.2° C. and a peak at 168.3° C. (FIG. 22).

Thus DSC analysis shows 2-[4-[4-[(5-fluoro-4-imidazo[1,2-a]pyridin-3-yl-pyrimidin-2-yl)amino]-3-methoxy-phenyl]piperazin-1-yl]ethanol glycolate salt is a high melting solid with an onset of melting at about 157.2° C. and a peak at 168.3° C.

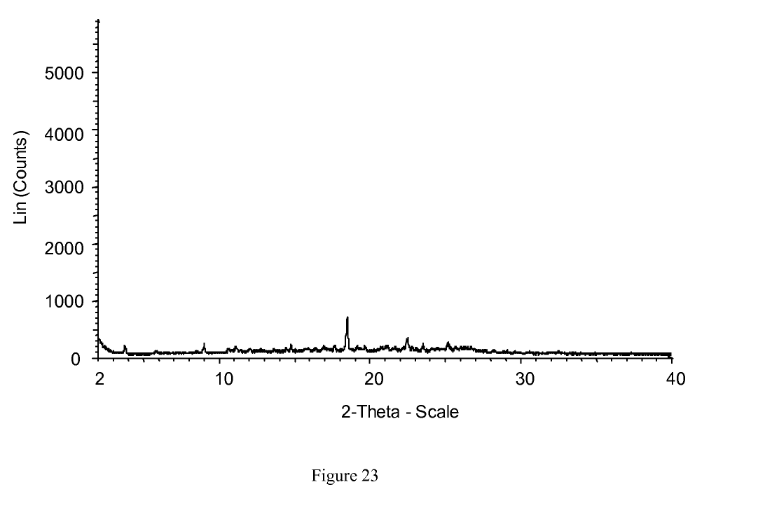

The present invention also provides a crystalline form of a 2-[4-[4-[(5-fluoro-4-imidazo[1,2-a]pyridin-3-yl-pyrimidin-2-yl)amino]-3-methoxy-phenyl]piperazin-1-yl]ethanol edisylate salt. The 2-[4-[4-[(5-fluoro-4-imidazo[1,2-a]pyridin-3-yl-pyrimidin-2-yl)amino]-3-methoxy-phenyl]piperazin-1-yl]ethanol edisylate salt is characterised in providing at least one of the following 2θ values measured using CuKa radiation: 18.5° and 22.5°. The 2-[4-[4-[(5-fluoro-4-imidazo[1,2-a]pyridin-3-yl-pyrimidin-2-yl)amino]-3-methoxy-phenyl]piperazin-1-yl]ethanol edisylate salt is characterised in providing an X-ray powder diffraction pattern, substantially as shown in FIG. 23. The ten most prominent peaks are shown in Table 13:

TABLE 13
Ten most Prominent X-Ray Powder Diffraction peaks for
2-[4-[4-[(5-fluoro-4-imidazo[1,2-a]pyridin-3-yl-pyrimidin-2-yl)amino]-
3-methoxy-phenyl]piperazin-1-yl]ethanol edisylate salt

| Angle 2- | | Relative |
|---|---|---|
| Theta (2θ) | Intensity % | Intensity |
| 18.466 | 100.0 | vs |
| 22.489 | 54.7 | vs |
| 22.549 | 41.0 | vs |
| 25.175 | 38.6 | vs |
| 23.525 | 37.8 | vs |
| 8.965 | 36.5 | vs |
| 19.121 | 35.2 | vs |
| 17.647 | 34.6 | vs |
| 21.053 | 33.3 | vs |
| 21.084 | 33.3 | vs |

vs = very strong

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol edisylate salt which has an X-ray powder diffraction pattern with at least one specific peak at about 2-theta=18.5°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol edisylate salt which has an X-ray powder diffraction pattern with at least one specific peak at about 2-theta=22.5°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol edisylate salt, which has an X-ray powder diffraction pattern with at least two specific peaks at about 2-theta=18.5° and 22.5°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol edisylate salt which has an X-ray powder diffraction pattern with specific peaks at about 2-theta=18.5°, 22.5°, 22.6°, 25.2°, 23.5°, 9.0°, 19.1°, 17.7°, 21.1°, 21.1°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol edisylate salt which has an X-ray powder diffraction pattern substantially the same as the X-ray powder diffraction pattern shown in FIG. 23.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol edisylate salt, which has an X-ray powder diffraction pattern with at least one specific peak at 2-theta=18.5° plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol edisylate salt which has an X-ray powder diffraction pattern with at least one specific peak at 2-theta=22.5° plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol edisylate salt, which has an X-ray powder diffraction pattern with at least two specific peaks at 2-theta=18.5° and 22.5° wherein said values may be plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol edisylate salt which has an X-ray powder diffraction pattern with specific peaks at 2-theta=18.5°, 22.5°, 22.6°, 25.2°, 23.5°, 9.0°, 19.1°, 17.7°, 21.1°, 21.1° wherein said values may be plus or minus 0.5° 2-theta.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol edisylate salt, which has an X-ray powder diffraction pattern with at least one specific peak at 2-theta=18.5°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol edisylate salt which has an X-ray powder diffraction pattern with at least one specific peak at 2-theta=22.5°.

According to the present invention there is provided a crystalline form, 2-[4-[4-[(5-fluoro-4-imidazo[1,2-a]pyridin-3-yl-pyrimidin-2-yl)amino]-3-methoxy-phenyl]piperazin-1-yl]ethanol edisylate salt which has an X-ray powder diffraction pattern with at least two specific peaks at 2-theta=18.5° and 22.5°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol edisylate salt which has an X-ray powder diffraction pattern with specific peaks at 2-theta=18.5°, 22.5°, 22.6°, 25.2°, 23.5°, 9.0°, 19.1°, 17.7°, 21.1°, 21.1°.

According to the present invention there is provided crystalline form, 2-[4-[4-[(5-fluoro-4-imidazo[1,2-a]pyridin-3-yl-pyrimidin-2-yl)amino]-3-methoxy-phenyl]piperazin-1-yl]ethanol edisylate salt, which has an X-ray powder diffraction pattern as shown in FIG. 23.

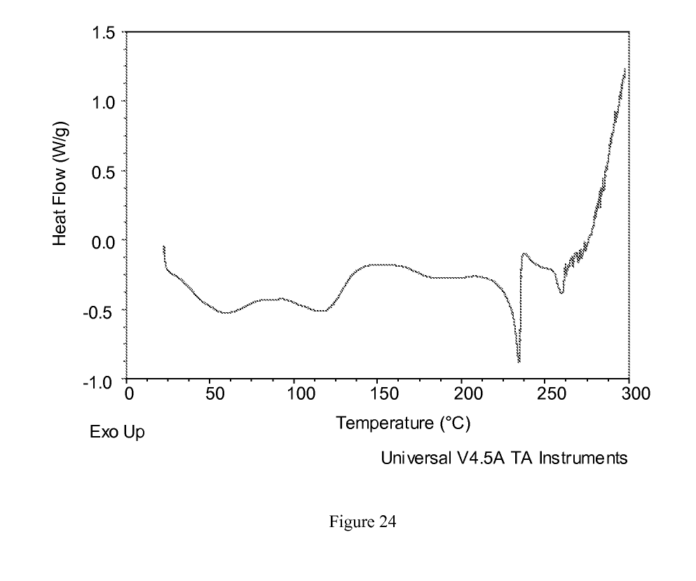

DSC analysis of 2-[4-[4-[(5-fluoro-4-imidazo[1,2-a]pyridin-3-yl-pyrimidin-2-yl)amino]-3-methoxy-phenyl]piperazin-1-yl]ethanol edisylate salt shows an initial event with an onset at 30.8° C. and a peak at 53.9° C. followed by a subsequent event with an onset at 96.4° C. and a peak at 121.7° C. This is followed by a melt with an onset of 229.3° C. and a peak at 234.5° C. (FIG. 24).

Thus DSC analysis shows 2-[4-[4-[(5-fluoro-4-imidazo[1,2-a]pyridin-3-yl-pyrimidin-2-yl)amino]-3-methoxy-phenyl]piperazin-1-yl]ethanol edisylate salt is a high melting solid with an onset of melting at about 229.3° C. and a peak at 234.5° C.

When it is stated that the present invention relates to a crystalline form of any of the specific compounds discussed above, the degree of crystallinity is conveniently greater than about 60%, more conveniently greater than about 80%, preferably greater than about 90% and more preferably greater than about 95%. Most preferably the degree of crystallinity is greater than about 98%.

It will be understood that the 2-theta values of an X-ray powder diffraction pattern may vary slightly from one machine to another or from one sample to another, and so the values quoted herein are not to be construed as absolute. It is known that an X-ray powder diffraction pattern may be obtained which has one or more measurement errors depending on measurement conditions (such as equipment or machine used). In particular, it is generally known that intensities in an X-ray powder diffraction pattern may fluctuate depending on measurement conditions. Therefore it should be understood that the Forms of the compounds of the present invention discussed above are not limited to the crystals that provide X-ray powder diffraction patterns identical to the X-ray powder diffraction pattern shown in FIGS. 1, 2 and 3 respectively, and any crystals providing X-ray powder diffraction patterns substantially the same as those shown in FIGS. 1, 2 and 3 fall within the scope of the present invention. A person skilled in the art of X-ray powder diffraction is able to judge the substantial identity of X-ray powder diffraction patterns.

Persons skilled in the art of X-ray powder diffraction will realise that the relative intensity of peaks can be affected by, for example, grains above 30 microns in size and non-unitary aspect ratios, which may affect analysis of samples. The skilled person will also realise that the position of reflections can be affected by the precise height at which the sample sits in the diffractometer and the zero calibration of the diffractometer. The surface planarity of the sample may also have a small effect. Hence the diffraction pattern data presented are not to be taken as absolute values. (Jenkins, R & Snyder, R. L. ‘Introduction to X-Ray Powder Diffractometry’ John Wiley & Sons 1996; Bunn, C. W. (1948), Chemical Crystallography, Clarendon Press, London; Klug, H. P. & Alexander, L. E. (1974), X-Ray Diffraction Procedures).

Generally, a measurement error of a diffraction angle in an X-ray powder diffractogram is approximately plus or minus 0.5° 2-theta, and such degree of a measurement error should be taken into account when considering the X-ray powder diffraction pattern in FIGS. 1, 2 and 3 and when reading the associated Tables. Furthermore, it should be understood that intensities might fluctuate depending on experimental conditions and sample preparation (preferred orientation).

A compound of formula (I), or a pharmaceutically acceptable salt thereof, may be prepared by any process known to be applicable to the preparation of chemically-related compounds. Such processes, when used to prepare a compound of formula (I) are provided as a further feature of the invention and are illustrated by the following representative process variants in which, unless otherwise stated, X, R², R³, R⁴ and q have any of the meanings defined hereinbefore. Necessary starting materials may be obtained by standard procedures of organic chemistry. The preparation of such starting materials is described in conjunction with the following representative process variants and within the accompanying Examples. Alternatively necessary starting materials are obtainable by analogous procedures to those illustrated which are within the ordinary skill of an organic chemist.

Process (a) the reaction, conveniently in the presence of a suitable acid, of a compound of formula (II):

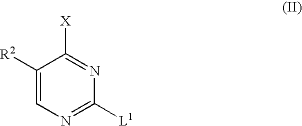

wherein L¹ represents a suitable displaceable group and X and R² are as defined in formula (I) except that any functional group is protected if necessary, with a compound of formula (III):

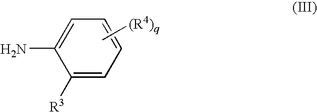

wherein R³, R⁴ and q are as defined in formula (I) except that any functional group is protected if necessary;

Process (b) the reaction, conveniently in the presence of a suitable base, of a compound of formula (II):

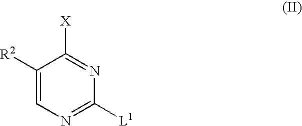

wherein L¹ represents a suitable displaceable group and X and R² are as defined in formula (I) except that any functional group is protected if necessary, with a compound of formula (IV):

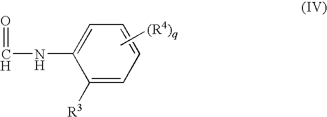

wherein R³, R⁴ and q are as defined in formula (I) except that any functional group is protected if necessary;

and optionally after process (a) or (b) carrying out one or more of the following:
converting the compound obtained to a further compound of the invention
forming a pharmaceutically acceptable salt of the compound.

### Process (a)

### Reaction Conditions for Process (a)

A suitable displaceable group L¹ in the compound of formula (II) is for example a halogeno or (C1-C4)alkylsulfonyl group, especially a halogeno group, for example a fluoro or chloro group. A particular group L¹ is chloro.

Process (a) conveniently may be carried out in the presence of a suitable acid. A suitable acid is, for example, hydrochloric acid or para-toluene sulfonic acid.

Process (a) may conveniently be carried out in the presence of a suitable inert solvent or diluent, for example dioxane, an alcohol such as 2-pentanol or 4-methyl-2-pentanol or a dipolar aprotic solvent such as N,N-dimethylformamide, N,N-dimethylacetamide, N-methylpyrrolidin-2-one or dimethylsulfoxide, and at a temperature in the range from 130 to 170° C.

Process (a) may alternatively conveniently be carried out under standard Buchwald conditions (see, for example, J. Am. Chem. Soc., 118, 7215; J. Am. Chem. Soc., 119, 8451; J. Org. Chem., 62, 1568 and 6066). For example, process (a) may conveniently be carried out in the presence of palladium acetate or tris(dibenzylideneacetone)dipalladium, in a suitable inert solvent or diluent for example 1,4-dioxane or an aromatic solvent such as toluene, benzene or xylene, in the presence of a suitable base, for example an inorganic base such as potassium carbonate or caesium carbonate or an organic base such as potassium-t-butoxide or 1,8-diazabicyclo[5.4.0]undec-7-ene (DBU) and in the presence of a suitable ligand such as 2,2′-bis(diphenylphosphino)-1,1′-binaphthyl or (9,9-dimethyl-9H-xanthene-4,5-diyl)bis(diphenylphosphine) and at a temperature in the range from 25 to 100° C. Typically, such a Buchwald reaction is conducted under an inert atmosphere, such as nitrogen or argon.

Conveniently, process (a) may be performed by heating the reactants in a sealed vessel using a suitable heating apparatus such as a microwave heater.

### Starting Materials for Process (a)

A compound of formula (II) may be obtained by conventional procedures. For example, an imidazopyridine compound of formula (II) may be obtained by the following reaction:

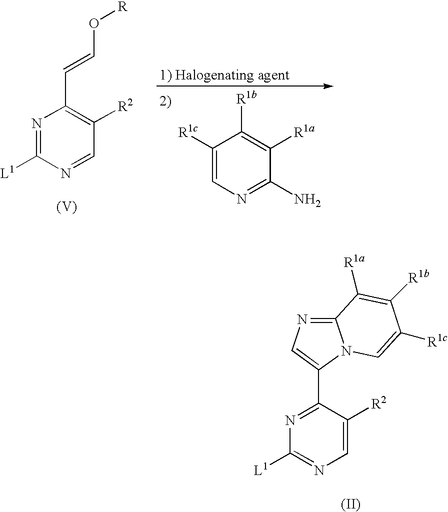

wherein L¹ represents a suitable displaceable group, R², R^{1a}, R^{1b} and R^{1c} are as defined in formula (I) except that any functional group is protected if necessary and R represents a (C1-C10)alkyl group. In step (1), the compound of formula (V) is reacted at room temperature with a halogenating agent (for example N-bromosuccinimide or bromine, preferably N-bromosuccinimide) in a suitable inert solvent or diluent, for example dioxane or an alcohol (such as ethanol), optionally in the presence of a co-solvent such as water. In step (2), the appropriate 2-aminopyridine is added and the reaction mixture is heated to a suitable temperature, typically of from about 80 to 100° C.

A compound of formula (V) may be obtained by conventional procedures. For example, a compound of formula (V) may be obtained by the following reaction:

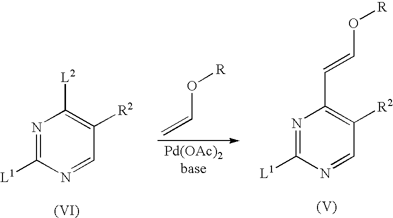

wherein L¹ and L² each represent a suitable displaceable group, R² is as defined in formula (I) except that any functional group is protected if necessary and R represents a (C1-C10)alkyl group. The compound of formula (VI) is reacted with an alkyl vinyl ether in the presence of palladium acetate and a suitable base (for example a tertiary amine, such as triethylamine) in a suitable inert solvent or diluent such as polyethyleneglycol (PEG). Further suitable details of the reaction conditions for this reaction may be found in Organic Letters, 2002, vol. 4, p. 4399. A suitable displaceable group L¹ is as discussed above and a suitable displaceable group L² is for example a halogeno group, for example a bromo or chloro group, particularly chloro.

A compound of formula (V) may alternatively be obtained by the following reaction:

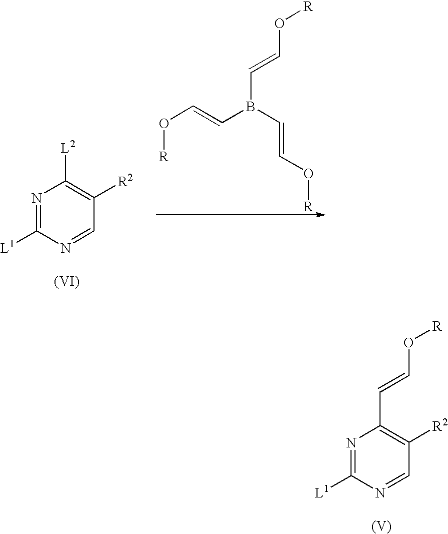

wherein L¹ and L² each represent a suitable displaceable group as discussed above, R² is as defined in formula (I) except that any functional group is protected if necessary and R represents a (C1-C10)alkyl group. The compound of formula (VI) is reacted with the tri(alkoxyvinyl)borane compound under typical Suzuki conditions well known to a person skilled in the art, for example in the presence of a suitable palladium catalyst (such as Pd(PPh₃)₄) and a suitable base (such as sodium hydroxide, tripotassium phosphate or potassium carbonate). The reaction is conducted in the presence of a suitable inert solvent or diluent (such as tetrahydrofuran or toluene) and the tri(alkoxyvinyl)borane reagent is prepared in situ from ethynyl alkyl ether and borane (as described in Journal of Organic Chemistry, 1982, vol. 47, p. 2117).

A pyrazolopyridine compound of formula (II) may for example be obtained by the following reaction:

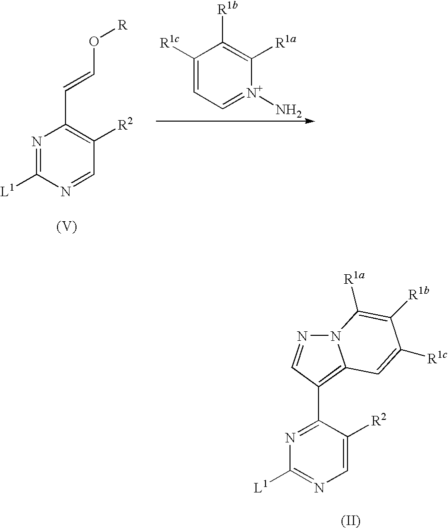

wherein L¹ represents a suitable displaceable group as discussed above, R², R^{1a}, R^{1b} and R^{1c} are as defined in formula (I) except that any functional group is protected if necessary and R represents a (C1-C10)alkyl group. The compound of formula (V) is reacted with a substituted 1-aminopyridinium salt in the presence of a suitable base (for example a tertiary amine, such as triethylamine). The reaction is conducted in the presence of a suitable inert solvent or diluent (for example an alcohol such as ethanol), in the presence of air and at a suitable temperature, such as from 80 to 100° C.

Alternatively, a pyrazolopyridine compound of formula (II) may be obtained by the following reaction:

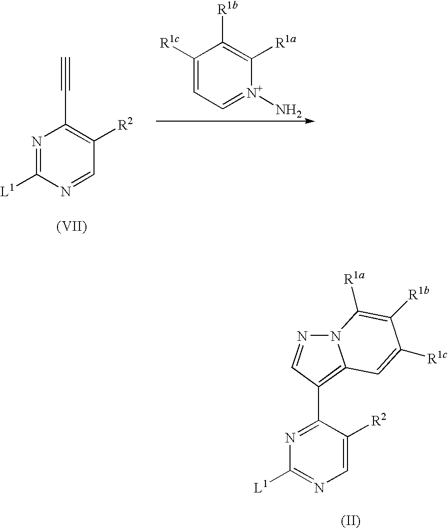

wherein L¹ represents a suitable displaceable group as discussed above and R², R^{1a}, R^{1b} and R^{1c} are as defined in formula (I) except that any functional group is protected if necessary. The reaction of the compound of formula (VII) with the substituted 1-aminopyridinium salt is conducted using similar reaction conditions to those discussed above for the reaction of a compound of formula (V) with the substituted 1-aminopyridinium salt

A compound of formula (VII) may be prepared by the reaction of a compound of formula (VI) with trimethylsilylacetylene using conditions for a Sonogashira coupling (see Organic Letters 2006, vol. 8, p. 269).

A compound of formula (III) may be obtained by conventional procedures. For example, a compound of formula (III) may be obtained by the reduction of a compound of formula (VIII):

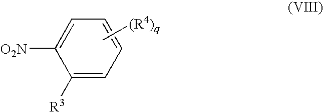

wherein R³, R⁴ and q are each as defined in formula (I) except that any functional group is protected if necessary. Any suitable conditions may be used for the reduction of a compound of formula (VIII). For example, the reduction may be conducted by reaction with hydrogen in the present of a suitable catalyst, such as platinum dioxide (also known as Adams catalyst) or palladium on charcoal.

Compounds of formulae (VI), (VII) and (VIII) are commercially available compounds or they are known in the literature, or they can be prepared by standard processes known in the art.

### Process (b)

### Reaction Conditions for Process (B)

A suitable displaceable group L¹ in the compound of formula (II) is for example a halogeno group or a (C1-C4)alkylsulfonyl group, especially a halogeno group, for example a fluoro or chloro group. A particular group L¹ is chloro.

Process (b) conveniently may be carried out in the presence of a suitable base. A suitable base is, for example, sodium hydride, sodium hexamethyldisilazane (NaHMDS), lithium hexamethyldisilazane (LiHMDS) or lithium diisopropylamide (LDA).

Process (b) may conveniently be carried out in the presence of a suitable inert solvent or diluent for example an ether such as tetrahydrofuran or 1,4-dioxane and at a suitable temperature such as ambient temperature.

### Starting Materials for Process (b)

Compounds of formula (IV) are commercially available compounds or they are known in the literature, or they can be prepared by standard processes known in the art.

It will be appreciated that compounds of formula (I) can be converted into further compounds of formula (I) using standard procedures conventional in the art, for example by means of conventional substitution reactions or of conventional functional group modifications either prior to or immediately following the processes mentioned above, and such procedures are included in the process aspect of the invention.

Examples of the types of conversion reactions that may be used include introduction of a substituent by means of an aromatic substitution reaction or of a nucleophilic substitution reaction, reduction of substituents, alkylation of substituents and oxidation of substituents. The reagents and reaction conditions for such procedures are well known in the chemical art.

Particular examples of aromatic substitution reactions include the introduction of a nitro group using concentrated nitric acid; the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of nucleophilic substitution reactions include the introduction of an alkoxy group or of an alkylamino group, a dialkyamino group or a N-containing heterocycle using standard conditions. Particular examples of reduction reactions include the reduction of a carbonyl group to a hydroxy group with sodium borohydride or of a nitro group to an amino group by catalytic hydrogenation with a nickel catalyst or by treatment with iron in the presence of hydrochloric acid with heating; and particular examples of oxidation reactions include oxidation of alkylthio to alkylsulfinyl or alkylsulfonyl. Other conversion reactions that may be used include the acid catalysed esterification of carboxylic acids with alcohols.

It will be appreciated that the preparation of compounds of formula (I) may involve, at various stages, the addition and removal of one or more protecting groups. The protecting groups used in the processes above may in general be chosen from any of the groups described in the literature or known to the skilled chemist as appropriate for the protection of the group in question and may be introduced by conventional methods. Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Specific examples of protecting groups are given below for the sake of convenience, in which “lower”, as in, for example, lower alkyl, signifies that the group to which it is applied preferably has 1 to 4 carbon atoms. It will be understood that these examples are not exhaustive. Where specific examples of methods for the removal of protecting groups are given below these are similarly not exhaustive. The use of protecting groups and methods of deprotection not specifically mentioned are, of course, within the scope of the invention.

A carboxy protecting group may be the residue of an ester-forming aliphatic or arylaliphatic alcohol or of an ester-forming silanol (the said alcohol or silanol preferably containing 1 to 20 carbon atoms). Examples of carboxy protecting groups include straight or branched chain (1 to 12C)alkyl groups (for example isopropyl, and tert-butyl); lower alkoxy-lower alkyl groups (for example methoxymethyl, ethoxymethyl and isobutoxymethyl); lower acyloxy-lower alkyl groups, (for example acetoxymethyl, propionyloxymethyl, butyryloxymethyl and pivaloyloxymethyl); lower alkoxycarbonyloxy-lower alkyl groups (for example 1-methoxycarbonyloxyethyl and 1-ethoxycarbonyloxyethyl); aryl-lower alkyl groups (for example benzyl, 4-methoxybenzyl, 2-nitrobenzyl, 4-nitrobenzyl, benzhydryl and phthalidyl); tri(lower alkyl)silyl groups (for example trimethylsilyl and tert-butyldimethylsilyl); tri(lower alkyl)silyl-lower alkyl groups (for example trimethylsilylethyl); and (2-6C)alkenyl groups (for example allyl). Methods particularly appropriate for the removal of carboxy protecting groups include for example acid-, base-, metal- or enzymically-catalysed cleavage.

Examples of hydroxy protecting groups include lower alkyl groups (for example tert-butyl), lower alkenyl groups (for example allyl); lower alkanoyl groups (for example acetyl); lower alkoxycarbonyl groups (for example tert-butoxycarbonyl); lower alkenyloxycarbonyl groups (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); tri(lower alkyl)silyl (for example trimethylsilyl and tert-butyldimethylsilyl) and aryl-lower alkyl (for example benzyl) groups.

Examples of amino protecting groups include formyl, aryl-lower alkyl groups (for example benzyl and substituted benzyl, 4-methoxybenzyl, 2-nitrobenzyl and 2,4-dimethoxybenzyl, and triphenylmethyl); di-4-anisylmethyl and furylmethyl groups; lower alkoxycarbonyl (for example tert-butoxycarbonyl); lower alkenyloxycarbonyl (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); lower alkanoyloxyalkyl groups (for example pivaloyloxymethyl); trialkylsilyl (for example trimethylsilyl and tert-butyldimethylsilyl); alkylidene (for example methylidene) and benzylidene and substituted benzylidene groups.

Methods appropriate for removal of hydroxy and amino protecting groups include, for example, acid-, base-, metal- or enzymically-catalysed hydrolysis for groups such as 2-nitrobenzyloxycarbonyl, hydrogenation for groups such as benzyl and photolytically for groups such as 2-nitrobenzyloxycarbonyl. For example a tert butoxycarbonyl protecting group may be removed from an amino group by an acid catalysed hydrolysis using trifluoroacetic acid.

The reader is referred to Advanced Organic Chemistry, 4th Edition, by J. March, published by John Wiley & Sons 1992, for general guidance on reaction conditions and reagents and to Protective Groups in Organic Synthesis, 2^{nd} Edition, by T. Green et al., also published by John Wiley & Son, for general guidance on protecting groups.

When a pharmaceutically-acceptable salt of a compound of formula (I) is required, for example an acid-addition salt, it may be obtained by, for example, reaction of said compound with a suitable acid using a conventional procedure. When it is desired to obtain the free base from a salt of the compound of formula (I), a solution of the salt may be treated with a suitable base, for example, an alkali or alkaline earth metal carbonate or hydroxide, for example sodium carbonate, potassium carbonate, calcium carbonate, sodium hydroxide or potassium hydroxide.

When a pharmaceutically acceptable salt of a compound of formula (I) is required, for example an acid-addition salt, it may be obtained by, for example, reaction of said compound with a suitable acid using a conventional procedure.

As mentioned hereinbefore some of the compounds according to the present invention may contain one or more chiral centers and may therefore exist as stereoisomers. Stereoisomers may be separated using conventional techniques, e.g. chromatography or fractional crystallisation. The enantiomers may be isolated by separation of a racemate for example by fractional crystallisation, resolution or HPLC. The diastereoisomers may be isolated by separation by virtue of the different physical properties of the diastereoisomers, for example, by fractional crystallisation, HPLC or flash chromatography. Alternatively particular stereoisomers may be made by chiral synthesis from chiral starting materials under conditions which will not cause racemisation or epimerisation, or by derivatisation, with a chiral reagent. When a specific stereoisomer is isolated it is suitably isolated substantially free for other stereoisomers, for example containing less than 20%, particularly less than 10% and more particularly less than 5% by weight of other stereoisomers.

In the section above relating to the preparation of the compounds of formula (I), the expression “inert solvent” refers to a solvent which does not react with the starting materials, reagents, intermediates or products in a manner which adversely affects the yield of the desired product.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

Certain novel intermediates utilised in the above processes are provided as a further feature of the present invention together with the process for their preparation. According to a further feature of the present invention there is provided the compounds of the Formulae V or a salt thereof (including pharmaceutically acceptable salts thereof) as hereinbefore defined.

The present inventors have found that the compounds of the present invention possess anti-proliferative properties such as anti-cancer properties that are believed to arise from their IGF-1R tyrosine kinase inhibitory activity. Furthermore, certain of the compounds according to the present invention possess substantially better potency against the IGF-1R tyrosine kinase than against other tyrosine kinases enzymes. Such compounds possess sufficient potency against the IGF-1R tyrosine kinase that they may be used in an amount sufficient to inhibit IGF-1R tyrosine kinase whilst demonstrating little, or significantly lower, activity against other tyrosine kinases. Such compounds are likely to be useful for the effective treatment of, for example, IGF-1R driven tumours.

Accordingly, the compounds of the present invention are expected to be useful in the treatment of diseases or medical conditions mediated alone or in part by IGF-1R tyrosine kinase, i.e. the compounds may be used to produce an IGF-1R tyrosine kinase modulatory or inhibitory effect in a warm-blooded animal in need of such treatment. Thus the compounds of the present invention provide a method for the treatment of malignant cells characterised by modulation or inhibition of the IGF-1R tyrosine kinase. Particularly the compounds of the invention may be used to produce an anti-proliferative and/or pro-apoptotic and/or anti-invasive effect mediated alone or in part by the modulation or inhibition of IGF-1R tyrosine kinase. Particularly, the compounds of the present invention are expected to be useful in the prevention or treatment of those tumours that are sensitive to modulation or inhibition of IGF-1R tyrosine kinase that is involved in the signal transduction steps which drive proliferation and survival of these tumour cells. Accordingly the compounds of the present invention are expected to be useful in the treatment and/or prevention of a number of proliferative and hyperproliferative diseases/conditions and the compounds of formula (I) have activity as a pharmaceutical, in particular as a modulator or inhibitor of insulin-like growth factor-1 receptor (IGF-1R) activity, and may be used in the treatment of proliferative and hyperproliferative diseases/conditions, examples of which include the following cancers:

(1) carcinoma, including that of the bladder, brain, breast, cervix, colon, endometrium, head, kidney, liver, lung, neck, oesophagus, ovary, pancreas, prostate, skin, stomach and thyroid;
(2) hematopoietic tumours of lymphoid lineage, including acute lymphocytic leukaemia, B-cell lymphoma and Burketts lymphoma;
(3) hematopoietic tumors of myeloid lineage, including acute and chronic myelogenous leukaemias and promyelocytic leukaemia;
(4) tumors of mesenchymal origin, including fibrosarcoma and rhabdomyosarcoma; and
(5) other tumors, including melanoma, seminoma, tetratocarcinoma, neuroblastoma and glioma.

The compound of the invention are especially useful in the treatment of tumors of the breast, prostate, lung and colorectal area.

According to a further aspect, therefore, the present invention provides a compound of formula (I), or a pharmaceutically acceptable salt thereof, as defined above for use in therapy of the human or animal body.

It will be appreciated that “therapy” also includes “prophylaxis” unless otherwise indicated. The terms “therapeutic” and “therapeutically” will be understood accordingly.

According to a further aspect of the present invention there is provided a compound of formula (I), or a pharmaceutically acceptable salt thereof, as defined above for use as a medicament.

In particular, the invention provides a compound of formula (I), or a pharmaceutically acceptable salt thereof, as defined above for use in modulating insulin-like growth factor-1 receptor (IGF-1R) activity in a human or animal.

The invention also provides the use of a compound of formula (I), or a pharmaceutically acceptable salt thereof, as defined above in modulating insulin-like growth factor-1 receptor (IGF-1R) activity in a human or animal.

The invention also provides the use of a compound of formula (I), or a pharmaceutically acceptable salt thereof, as hereinbefore defined in the manufacture of a medicament for use in therapy, in particular in modulating insulin-like growth factor-1 receptor (IGF-1R) activity in a human or animal.

The invention further provides a method of modulating insulin-like growth factor-1 receptor (IGF-1R) activity which comprises administering to a patient in need thereof a therapeutically effective amount of a compound of formula (I), or a pharmaceutically acceptable salt thereof, as hereinbefore defined.

The invention also provides a compound of formula (I), or a pharmaceutically acceptable salt thereof, for use in the production of an anti-proliferative effect in a human or animal.

The invention also provides the use of a compound of formula (I), or a pharmaceutically acceptable salt thereof, in the production of an anti-proliferative effect in a human or animal.

The invention also provides the use of a compound of formula (I), or a pharmaceutically acceptable salt thereof, as defined hereinbefore in the manufacture of a medicament for use in the production of an anti-proliferative effect in a human or animal.

The invention also provides a method for producing an anti-proliferative effect which comprises administering to a patient in need thereof a therapeutically effective amount of a compound of formula (I), or a pharmaceutically acceptable salt thereof, as hereinbefore defined.

The invention also provides a compound of formula (I), or a pharmaceutically-acceptable salt thereof, for use in the production of an anti-proliferative effect which effect is produced alone or in part by inhibiting insulin-like growth factor-1 receptor (IGF-1R) tyrosine kinase in a human or animal.

The invention also provides the use of a compound of formula (I), or a pharmaceutically-acceptable salt thereof, in the production of an anti-proliferative effect which effect is produced alone or in part by inhibiting insulin-like growth factor-1 receptor (IGF-1R) tyrosine kinase in a human or animal.

The invention also provides the use of a compound of formula (I), or a pharmaceutically-acceptable salt thereof, as defined hereinbefore in the manufacture of a medicament for use in the production of an anti-proliferative effect which effect is produced alone or in part by inhibiting insulin-like growth factor-1 receptor (IGF-1R) tyrosine kinase in a human or animal.

The invention also provides a method for producing an anti-proliferative effect which effect is produced alone or in part by inhibiting insulin-like growth factor-1 receptor (IGF-1R) tyrosine kinase which comprises administering to a patient in need thereof a therapeutically effective amount of a compound of formula (I), or a pharmaceutically-acceptable salt thereof, as hereinbefore defined.

The invention also provides a compound of formula (I), or a pharmaceutically-acceptable salt thereof, for use in the treatment of a disease or medical condition (for example a cancer as mentioned herein) mediated alone or in part by insulin-like growth factor-1 receptor (IGF-1R) tyrosine kinase.

The invention also provides the use of a compound of formula (I), or a pharmaceutically-acceptable salt thereof, in the treatment of a disease or medical condition (for example a cancer as mentioned herein) mediated alone or in part by insulin-like growth factor-1 receptor (IGF-1R) tyrosine kinase.

The invention also provides the use of a compound of formula (I), or a pharmaceutically-acceptable salt thereof, as defined hereinbefore in the manufacture of a medicament for use in the treatment of a disease or medical condition (for example a cancer as mentioned herein) mediated alone or in part by insulin-like growth factor-1 receptor (IGF-1R) tyrosine kinase.

The invention also provides a method for treating a disease or medical condition (for example a cancer as mentioned herein) mediated alone or in part by insulin-like growth factor-1 receptor (IGF-1R) tyrosine kinase which comprises administering to a patient in need thereof a therapeutically effective amount of a compound of formula (I), or a pharmaceutically-acceptable salt thereof, as defined hereinbefore.

The invention also provides a compound of formula (I), or a pharmaceutically-acceptable salt thereof, for use in the prevention or treatment of those tumours which are sensitive to inhibition of insulin-like growth factor-1 receptor (IGF-1R) tyrosine kinase, involved in the signal transduction steps which lead to the proliferation and/or survival of tumour cells.

The invention also provides the use of a compound of formula (I), or a pharmaceutically-acceptable salt thereof, in the prevention or treatment of those tumours which are sensitive to inhibition of insulin-like growth factor-1 receptor (IGF-1R) tyrosine kinase, involved in the signal transduction steps which lead to the proliferation and/or survival of tumour cells.

The invention also provides the use of a compound of formula (I), or a pharmaceutically-acceptable salt thereof, as defined hereinbefore in the manufacture of a medicament for use in the prevention or treatment of those tumours which are sensitive to inhibition of insulin-like growth factor-1 receptor (IGF-1R) tyrosine kinase involved in the signal transduction steps which lead to the proliferation of tumour cells.

The invention also provides a method for the prevention or treatment of those tumours which are sensitive to inhibition of insulin-like growth factor-1 receptor (IGF-1R) tyrosine kinase, involved in the signal transduction steps which lead to the proliferation and/or survival of tumour cells which comprises administering to a patient in need thereof a therapeutically effective amount of a compound of formula (I), or a pharmaceutically-acceptable salt thereof, as defined hereinbefore.

The invention also provides a compound of formula (I), or a pharmaceutically-acceptable salt thereof, for use in providing an insulin-like growth factor-1 receptor (IGF-1R) tyrosine kinase inhibitory effect.

The invention also provides the use of a compound of formula (I), or a pharmaceutically-acceptable salt thereof, in providing an insulin-like growth factor-1 receptor (IGF-1R) tyrosine kinase inhibitory effect.

The invention also provides the use of a compound of formula (I), or a pharmaceutically-acceptable salt thereof, as defined hereinbefore in the manufacture of a medicament for use in providing an insulin-like growth factor-1 receptor (IGF-1R) tyrosine kinase inhibitory effect.

The invention also provides a method for providing an insulin-like growth factor-1 receptor (IGF-1R) tyrosine kinase inhibitory effect which comprises administering to a patient in need thereof a therapeutically effective amount of a compound of formula (I), or a pharmaceutically-acceptable salt thereof, as defined hereinbefore.

The invention also provides a compound of formula (I), or a pharmaceutically acceptable salt thereof, as hereinbefore defined for use in the treatment of cancer.

The invention also provides the use of a compound of formula (I), or a pharmaceutically acceptable salt thereof, as hereinbefore defined in the treatment of cancer.

The invention also provides the use of a compound of formula (I), or a pharmaceutically acceptable salt thereof, as hereinbefore defined in the manufacture of a medicament for use in the treatment of cancer.

The invention also provides a method of treating cancer which comprises administering to a patient in need thereof a therapeutically effective amount of a compound of formula (I), or a pharmaceutically acceptable salt thereof, as hereinbefore defined.

The invention also provides a compound of formula (I), or a pharmaceutically acceptable salt thereof, as hereinbefore defined for use in the treatment of cancer of the prostate, lung, colorectal area or breast.

The invention also provides the use of a compound of formula (I), or a pharmaceutically acceptable salt thereof, as hereinbefore defined in the treatment of cancer of the prostate, lung, colorectal area or breast.

The invention also provides the use of a compound of formula (I), or a pharmaceutically acceptable salt thereof, as hereinbefore defined in the manufacture of a medicament for use in the treatment of cancer of the prostate, lung, colorectal area or breast.

The invention also provides a method of treating cancer of the prostate, lung, colorectal area or breast which comprises administering to a patient in need thereof a therapeutically effective amount of a compound of formula (I), or a pharmaceutically acceptable salt thereof, as hereinbefore defined.

The compounds of formula (I) may also be administered in the form of a prodrug which is broken down in the human or animal body to give a compound of formula (I). Various forms of prodrugs are known in the art. For examples of such prodrug derivatives, see:

a) Design of Prodrugs, edited by H. Bundgaard, (Elsevier, 1985) and Methods in Enzymology, Vol. 42, p. 309-396, edited by K. Widder, et al. (Academic Press, 1985);

b) A Textbook of Drug Design and Development, edited by Krogsgaard-Larsen and H. Bundgaard, Chapter 5 “Design and Application of Prodrugs”, by H. Bundgaard p. 113-191 (1991);

c) H. Bundgaard, Advanced Drug Delivery Reviews, 8, 1-38 (1992);

d) H. Bundgaard, et al., Journal of Pharmaceutical Sciences, 77, 285 (1988); and

e) N. Kakeya, et al., Chem Pharm Bull, 32, 692 (1984).

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I), or a pharmaceutically acceptable salt thereof, as hereinbefore defined, in association with a pharmaceutically acceptable adjuvant, diluent or carrier.

The invention further provides a process for the preparation of a pharmaceutical composition of the invention which comprises mixing a compound of formula (I), or a pharmaceutically acceptable salt thereof, as hereinbefore defined, with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane aerosols and dry powder formulations; or systemically, e.g. by oral administration in the form of tablets, capsules, syrups, solutions, suspensions, powders or granules; or by parenteral administration in the form of solutions or suspensions; or by subcutaneous administration; or by rectal administration in the form of suppositories; or transdermally. Suitable suspensions may include microsuspensions and nanosuspensions.

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

It will be appreciated that the dosage administered will vary depending on the compound employed, the mode of administration, the treatment desired and the disorder indicated. Typically a daily dose of active ingredient in the range of from 0.5 mg to 75 mg active ingredient per kg body weight is received, given if required in divided doses, the precise amount of compound received and the route of administration depending on the weight, age, sex of the patient being treated and on the particular disease condition being treated according to principles known in the art.

For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The anti cancer treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5 fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5*-reductase such as finasteride;

(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);

(iv) inhibitors of growth factor function: for example such inhibitors include growth factor antibodies and growth factor receptor antibodies (for example the anti erbB2 antibody trastuzumab [Herceptin™], the anti-EGFR antibody panitumumab, the anti erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI 774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;

(v) antiangiogenic agents such as those which inhibit the effects of vascular endothelial growth factor, [for example the anti vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856 and WO 98/13354 and compounds that work by other mechanisms (for example linomide, inhibitors of integrin avb3 function and angiostatin)];

(vi) vascular damaging agents such as Combretastatin A4 and compounds disclosed in International Patent Applications WO 99/02166, WO 00/40529, WO 00/41669, WO 01/92224, WO 02/04434 and WO 02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi drug resistance gene therapy; and

(ix) immunotherapy approaches, including for example ex vivo and in vivo approaches to increase the immunogenicity of patient tumour cells, such as transfection with cytokines such as interleukin 2, interleukin 4 or granulocyte macrophage colony stimulating factor, approaches to decrease T cell anergy, approaches using transfected immune cells such as cytokine transfected dendritic cells, approaches using cytokine transfected tumour cell lines and approaches using anti idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a pharmaceutical product comprising a compound of formula (I), or a pharmaceutically acceptable salt thereof, as defined hereinbefore and an additional anti-tumour agent as defined hereinbefore for the conjoint treatment of cancer.

Although the compounds of formula (I) are primarily of value as therapeutic agents for use in warm-blooded animals (including man), they are also useful whenever it is required to inhibit the effects of IGF-1R tyrosine kinases. Thus, they are useful as pharmacological standards for use in the development of new biological tests and in the search for new pharmacological agents.

The activity and selectivity of compounds according to the invention may be determined using an appropriate assay as described, for example, in WO 03/048133, and as detailed below.

### EXAMPLES

The invention will now be further described with reference to the following illustrative examples, in which, unless stated otherwise:

(i) temperatures are given in degrees Celsius (° C.); operations were carried out at room or ambient temperature, that is, at a temperature in the range of 18 to 25° C.;
(ii) organic solutions were dried over anhydrous magnesium sulfate; evaporation of solvent was carried out using a rotary evaporator under reduced pressure (600-4000 Pascals; 4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC or by HPLC and reaction times are given for illustration only;
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 500 MHz, in DMSO-d₆ unless otherwise indicated. The following abbreviations have been used: s, singlet; d, doublet; t, triplet; q, quartet; m, multiplet; b, broad;
(viii) chemical symbols have their usual meanings; SI units and symbols are used;
(ix) solvent ratios are given in volume:volume (v/v) terms;
(x) mass spectra were run using electrospray ionization (ESP); values for m/z are given; generally, only ions which indicate the parent mass are reported; and unless otherwise stated, the mass ion quoted is (MH)⁺; and
(xi) the following abbreviations have been used:
THF tetrahydrofuran;
EtOAc ethyl acetate;
DCM dichloromethane;
DMSO dimethylsulfoxide;
DIPEA diisopropylethylamine;
NMP N-methylpyrrolid-2-one;
MeOH methanol;
EtOH ethanol;
tBuOH tert-butyl alcohol;
TFA trifluoroacetic acid;
DMF dimethylformamide;
DMA N,N-dimethylacetamide;
pTsOH para-toluene sulfonic acid;
Ac acetyl;
DME dimethoxyethane;
OAc acetate.
   (xii) the Figures are as follows:

FIG. 1 shows the X-Ray Powder Diffraction Pattern for Form A of 1-[4-[4-[(5-chloro-4-imidazo[1,2-a]pyridin-3-yl-pyrimidin-2-yl)amino]-3-methoxy-phenyl]piperazin-1-yl]ethanone (see Example 1);

FIG. 2 shows the X-Ray Powder Diffraction Pattern for Form A of 2-[4-[4-[(5-fluoro-4-imidazo[1,2-a]pyridin-3-yl-pyrimidin-2-yl)amino]-3-methoxy-phenyl]piperazin-1-yl]ethanol (see Example 81); and

FIG. 3 shows the X-Ray Powder Diffraction Pattern for Form A of 2-[4-[4-[(4-imidazo[1,2-a]pyridin-3-yl-5-methyl-pyrimidin-2-yl)amino]-3-methoxy-phenyl]piperazin-1-yl]ethanol (see Example 101).

### Example 1

### 1-[4-[4-[(5-chloro-4-imidazo[1,2-a]pyridin-3-yl-pyrimidin-2-yl)amino]-3-methoxy-phenyl]piperazin-1-yl]ethanone

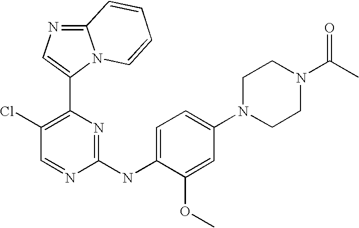

3-(2,5-dichloropyrimidin-4-yl)imidazo[1,2-a]pyridine (see Method 1, 100 mg, 0.38 mmol), 1-(4-(4-amino-3-methoxyphenyl)piperazin-1-yl)ethanone (see Method 7, 103 mg, 0.41 mmol), p-toluenesulfonic acid (71.8 mg, 0.38 mmol) were suspended in 2-pentanol (1.2 mL) and heated at 115° C. for 3 hours under argon. The reaction mixture was allowed to cool to room temperature, diluted with water, basified with ammonium hydroxide (pH=10) and extracted with dichloromethane. The combined organic layers were dried over magnesium sulfate, concentrated to dryness and purified by preparative HPLC using a Waters X-Terra reverse-phase column (5 microns silica, 30 mm diameter, 150 mm length) and decreasingly polar mixtures of water (containing 0.2% ammonium carbonate) and acetonitrile as eluent. The fractions were evaporated to dryness to afford the title compound (78 mg, 43%) as a yellow solid; NMR spectrum: 2.07 (s, 3H), 3.12-3.18 (m, 2H), 3.18-3.25 (m, 2H), 3.57-3.67 (m, 4H), 3.77 (s, 3H), 6.56 (dd, 1H), 6.74 (d, 1H), 6.89 (bs, 1H), 7.30 (d, 1H), 7.49 (ddd, 1H), 7.76 (d, 1H), 8.46 (s, 1H), 8.72 (s, 1H), 8.81 (s, 1H), 9.55 (bs, 1H; Mass spectrum: 478 (MH+).

An alternative method for the preparation of 1-[4-[4-[(5-chloro-4-imidazo[1,2-a]pyridin-3-yl-pyrimidin-2-yl)amino]-3-methoxy-phenyl]piperazin-1-yl]ethanone is as follows:

3-(2,5-dichloropyrimidin-4-yl)imidazo[1,2-a]pyridine (1.8 g, 6.79 mmol), 1-(4-(4-amino-3-methoxyphenyl)piperazin-1-yl)ethanone (1.69 g, 6.79 mmol) and p-toluenesulfonic acid hydrate (1.29 g, 6.79 mmol) were suspended in 2-pentanol (15 ml). The reaction mixture was heated at 140° C. for 5 hours then cooled and diluted with methylene chloride. The mixture was neutralised with an aqueous solution of potassium carbonate and the organic layer was separated, dried over magnesium sulfate and concentrated to afford the crude product (3.52 g). The crude product was purified by flash chromatography on silica gel eluting with 0 to 4% methanol in methylene chloride to afford 1-(4-(4-(5-chloro-4-(imidazo[1,2-a]pyridin-3-yl)pyrimidin-2-ylamino)-3-methoxyphenyl)piperazin-1-yl)ethanone as a yellow solid which was recrystallised from methylene chloride/diethylether (50/5 ml) providing 1.68 g of pure compound (51% yield). The recrystallised compound obtained was characterised by X-ray powder diffraction and Differential Scanning Calorimetry (DSC) according to the procedures set out below. The X-ray powder diffraction showed the solid to have a crystalline form, Form A, which has an X-ray powder diffraction pattern as shown in FIG. 1. The ten most prominent peaks are as follows:

| Angle 2- | | Relative |
|---|---|---|
| Theta (2θ) | Intensity % | Intensity |
| 15.758 | 100.0 | vs |
| 23.182 | 75.1 | vs |
| 24.423 | 71.6 | vs |
| 4.090 | 59.9 | vs |
| 14.537 | 36.0 | vs |
| 11.754 | 31.5 | vs |
| 9.975 | 29.1 | vs |
| 28.420 | 28.0 | vs |
| 26.580 | 27.7 | vs |
| 18.803 | 27.0 | vs |

vs = very strong,
s = strong

The DSC analysis of the crystalline form, Form A, showed a single event with an onset at 187.1° C. and a peak at 193.1° C. Thus DSC analysis showed that the crystalline form, Form A, is a high melting solid with an onset of melting at about 187.1° C. and a peak at 193.1° C.

### Example 2

### 5-chloro-4-imidazo[1,2-a]pyridin-3-yl-N-(2-methoxy-4-piperazin-1-yl-phenyl)pyrimidin-2-amine

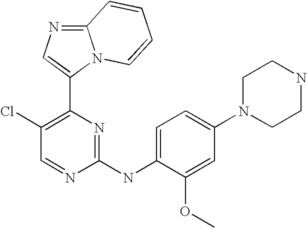

3-(2,5-dichloropyrimidin-4-yl)imidazo[1,2-a]pyridine (see Method 1, 120 mg, 0.45 mmol), 1-(4-(4-amino-3-methoxyphenyl)piperazin-1-yl)ethanone (see Method 7, 125 mg, 0.50 mmol), p-toluenesulfonic acid (172 mg, 0.91 mmol) in 4-methyl-2-pentanol (1.5 ml) were stirred at 150° C. for 3 hours. The reaction mixture was neutralised with a solution of ammonia in methanol, concentrated in vacuo and purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent. The resulting solid was triturated with ether, filtered and dried under vacuum to afford the title compound (124 mg, 45%) as a yellow solid; NMR spectrum: 2.36 (bs, 1H), 2.83-2.91 (m, 4H), 3.06-3.14 (m, 4H), 3.75 (s, 3H), 6.51 (dd, 1H), 6.68 (d, 1H), 6.88 (bs, 1H), 7.25 (d, 1H), 7.49 (ddd, 1H), 7.75 (d, 1H), 8.45 (s, 1H), 8.72 (s, 1H), 8.78 (s, 1H), 9.54 (bs, 1H); Mass spectrum: 436 (MH+).

Alternatively this compound was also prepared using the following procedure:

Sodium hydroxide (3.97 g, 99.1 mmol) was added to a stirred solution of 1-(4-(4-(5-chloro-4-(imidazo[1,2-a]pyridin-3-yl)pyrimidin-2-ylamino)-3-methoxyphenyl)piperazin-1-yl)ethanone (Example 1, 2.37 g, 4.96 mmol) dissolved in methanol (40 mL) and water (20 mL). The resulting suspension was stirred at 100° C. for 6 hours. Methanol was evaporated, a saturated aqueous solution of ammonium chloride was added and the mixture was extracted with dichloromethane. The organic phase was dried over magnesium sulfate and concentrated to afford the title compound (2.2 g, 100%) as a yellow foam.

### Example 3

The following compounds were prepared using a similar procedure to that described in Example 1 using 3-(2,5-dichloropyrimidin-4-yl)imidazo[1,2-a]pyridine and the appropriate aniline. The anilines used as a starting material are indicated in the following table and were prepared as described in the Methods section.

| Ex. No. | Name | Starting aniline | MH+ | NMR spectrum | % Yield |
|---|---|---|---|---|---|
| 3.1^{b} | [4-[(5-chloro-4- | (4-amino-3- | 465 | 3.54 (bs, 4H), 3.62 (bs, | 41 |
| | imidazo[1,2-a]pyridin- | methoxyphenyl)- | | 4H), 3.85 (s, 3H), | |
| | 3-yl- | morpholin-4-yl | | 7.00-7.06 (m, 2H), 7.16 (d, 1H), | |
| | pyrimidin-2- | methanone | | 7.53 (ddd, 1H), | |
| | yl)amino]-3- | (described in | | 7.76-7.81 (m, 2H), 8.58 (s, 1H), | |
| | methoxy-phenyl]- | WO2004/080980) | | 8.74 (s, 1H), 9.02 (s, 1H), | |
| | morpholino- | | | 9.62 (d, 1H) | |
| | methanone | | | | |
| 3.2^{b} | [1-[4-[(5-chloro-4- | [1-(4-amino-3- | 534 | 1.89-1.98 (m, 1H), | 41 |
| | imidazo[1,2-a]pyridin- | methoxyphenyl)pyrrolidin- | | 1.98-2.07 (m, 2H), | |
| | 3-yl- | 2-yl]- | | 2.24-2.34 (m, 1H), 3.37-3.46 (m, | |
| | pyrimidin-2- | morpholin-4-yl | | 2H), 3.46-3.68 (m, 7H), | |
| | yl)amino]-3- | methanone | | 3.70 (s, 3H), 3.75-3.84 (m, | |
| | methoxy- | (Method 9) | | 1H), 4.80 (dd, 1H), | |
| | phenyl]pyrrolidin- | | | 6.03 (dd, 1H), 6.13 (d, 1H), | |
| | 2-yl]-morpholino- | | | 6.94 (bs, 1H), 7.12 (d, 1H), | |
| | methanone | | | 7.46 (dd, 1H), 7.73 (d, | |
| | | | | 1H), 8.41 (s, 1H), 8.70 (s, | |
| | | | | 1H), 8.75 (s, 1H), 9.50 (bs, | |
| | | | | 1H) | |
| 3.3^{b} | 2-[4-[4-[(5-chloro- | 2-[4-(4-amino-3- | 563 | 2.58-2.64 (m, 4H), | 22 |
| | 4-imidazo[1,2-a]pyridin- | methoxyphenyl)piperazin- | | 3.15-3.23 (m, 4H), 3.24 (s, 2H), | |
| | 3-yl- | 1-yl]-1- | | 3.43-3.49 (m, 2H), | |
| | pyrimidin-2-yl)amino]- | morpholin-4-yl | | 3.53-3.65 (m, 6H), 3.76 (s, 3H), | |
| | 3-methoxy- | ethanone | | 6.51 (dd, 1H), 6.70 (d, | |
| | phenyl]piperazin-1- | (Method 7) | | 1H), 6.88 (bs, 1H), 7.27 (d, | |
| | yl]-1-morpholino- | | | 1H), 7.49 (dd, 1H), | |
| | ethanone | | | 7.75 (d, 1H), 8.45 (s, 1H), | |
| | | | | 8.72 (s, 1H), 8.78 (s, 1H), | |
| | | | | 9.55 (bs, 1H) | |
| 3.4^{b} | [3-[(5-chloro-4- | (3-amino-4- | 465 | 3.48 (bs, 4H), 3.548 (bs, | 22 |
| | imidazo[1,2-a]pyridin- | methoxyphenyl)- | | 4H), 3.87 (s, 3H), 7.01 (dd, | |
| | 3-yl- | morpholin-4-yl | | 1H), 7.19 (d, 1H), | |
| | pyrimidin-2-yl)amino]- | methanone | | 7.28 (dd, 1H), 7.52 (dd, 1H), | |
| | 4-methoxy- | (described in | | 7.78 (d, 1H), 7.81 (s, 1H), | |
| | phenyl]- | WO2007/140222) | | 8.57 (s, 1H), 8.73 (s, 1H), | |
| | morpholino- | | | 9.00 (s, 1H), 9.63 (bs, 1H) | |
| | methanone | | | | |
| 3.5^{b} | (3S)-1-[3-[(5- | (3S)-1-(3-amino-4- | 437 | 1.79-1.87 (m, 1H), | 28 |
| | chloro-4- | methoxyphenyl)pyrrolidin- | | 1.95-2.05 (m, 1H), 2.99 (dd, | |
| | imidazo[1,2-a]pyridin- | 3-ol | | 1H), 3.16 (ddd, 1H), | |
| | 3-yl- | (Method 8) | | 3.24 (dd, 1H), 3.32-3.37 (m | |
| | pyrimidin-2-yl)amino]- | | | partially hidden by H2O, | |
| | 4-methoxy- | | | 1H), 3.69 (s, 3H), | |
| | phenyl]pyrrolidin- | | | 4.32-4.38 (m, 1H), 4.90 (d, 1H), | |
| | 3-ol | | | 6.36 (dd, 1H), 6.83 (bs, | |
| | | | | 1H), 6.91 (dd, 1H), | |
| | | | | 7.00 (d, 1H), 7.48 (ddd, 1H), | |
| | | | | 7.75 (d, 1H), 8.52 (s, 1H), | |
| | | | | 8.71 (s, 1H), 8.85 (s, 1H), | |
| | | | | 9.60 (bs, 1H) | |
| 3.6 | 5-chloro-N-[4-(3,5- | 4-(3,5-dimethylpiperazin- | 464 | 1.05 (d, 6H), 2.11-2.21 (m, | 33 |
| | dimethylpiperazin- | 1-yl)-2- | | 2H), 2.83-2.93 (m, 2H), | |
| | 1-yl)-2-methoxy- | methoxyaniline | | 3.55-3.62 (m, 2H), 3.76 (s, | |
| | phenyl]-4- | (Method 7) | | 3H), 6.51 (dd, 1H), | |
| | imidazo[1,2-a]pyridin- | | | 6.68 (d, 1H), 6.87 (bs, 1H), | |
| | 3-yl- | | | 7.25 (d, 1H), 7.49 (dd, 1H), | |
| | pyrimidin-2-amine | | | 7.75 (d, 1H), 8.44 (s, 1H), | |
| | | | | 8.72 (s, 1H), 8.76 (s, 1H), | |
| | | | | 9.55 (bs, 1H) | |
| 3.7 | 4-[4-[(5-chloro-4- | 4-(4-amino-3- | 543 | 2.83 (s, 6H), 3.24-3.28 (m, | 12 |
| | imidazo[1,2- | methoxyphenyl)- | | 4H), 3.31-3.34 (m partially | |
| | a]pyridin-3-yl- | N,N-dimethylpiperazine- | | hidden by H2O, 4H), | |
| | pyrimidin-2-yl)amino]- | 1- | | 3.77 (s, 3H), 6.57 (dd, 1H), | |
| | 3-methoxy- | sulfonamide | | 6.75 (d, 1H), 6.89 (bs, 1H), | |
| | phenyl]-N,N- | (Method 7) | | 7.31 (d, 1H), 7.50 (ddd, 1H), | |
| | dimethyl- | | | 7.76 (d, 1H), 8.46 (s, 1H), | |
| | piperazine-1- | | | 8.72 (s, 1H), 8.81 (s, 1H), | |
| | sulfonamide | | | 9.55 (bs, 1H) | |
| 3.8^{b} | 5-chloro-4- | 2-methoxy-5- | 421 | 1.83-1.95 (m, 4H), | 22 |
| | imidazo[1,2-a]pyridin- | pyrrolidin-1- | | 3.08-3.18 (m, 4H), 3.70 (s, 3H), | |
| | 3-yl-N-(2- | ylaniline(Method | | 6.39 (dd, 1H), | |
| | methoxy-5- | 8) | | 6.84-6.92 (m, 2H), 7.00 (d, 1H), | |
| | pyrrolidin-1-yl- | | | 7.49 (ddd, 1H), 7.76 (d, 1H), | |
| | phenyl)pyrimidin- | | | 8.52 (s, 1H), 8.71 (s, 1H), | |
| | 2-amine | | | 8.84 (s, 1H), 9.58 (bs, 1H) | |
| 3.9^{b} | 1-[4-[3-[(5-chloro- | 1-[4-(3-amino-4- | 478 | 2.01 (s, 3H), 2.91-2.97 (m, | 7 |
| | 4-imidazo[1,2-a]pyridin- | methoxyphenyl)piperazin- | | 2H), 2.97-3.02 (m, 2H), | |
| | 3-yl- | 1-yl]ethanone | | 3.47-3.55 (m, 4H), 3.74 (s, | |
| | pyrimidin-2- | (Method | | 3H), 6.80 (dd, 1H), | |
| | yl)amino]-4- | 8) | | 6.95 (dd, 1H), 7.03 (d, 1H), | |
| | methoxy-phenyl]piperazin- | | | 7.33 (d, 1H), 7.50 (ddd, | |
| | 1-yl]ethanone | | | 1H), 7.77 (ddd, 1H), | |
| | | | | 8.54 (s, 1H), 8.71 (s, 1H), | |
| | | | | 8.95 (s, 1H), 9.57 (bs, 1H) | |
| 3.10^{b} | 5-chloro-4- | See note (c) | 436 | 2.76-2.83 (m, 4H), | 45 |
| | imidazo[1,2-a]pyridin- | | | 2.88-2.95 (m, 4H), 3.73 (s, 3H), | |
| | 3-yl-N-(2- | | | 6.76 (dd, 1H), 6.95 (dd, | |
| | methoxy-5- | | | 1H), 7.01 (d, 1H), 7.27 (d, | |
| | piperazin-1-yl- | | | 1H), 7.50 (ddd, 1H), | |
| | phenyl)pyrimidin- | | | 7.77 (ddd, 1H), 8.53 (s, 1H), | |
| | 2-amine | | | 8.70 (s, 1H), 8.83 (s, 1H), | |
| | | | | 9.55 (bs, 1H) | |
| 3.11 | 5-chloro-N-[4- | (3R)-1-(4-amino-3- | 464 | 1.80-1.91 (m, 1H), | 77 |
| | [(3R)-3- | methoxyphenyl)- | | 2.14-2.23 (m, 1H), 2.23 (s, 6H), | |
| | dimethylaminopyrrolidin- | N,N-dimethylpyrrolidin- | | 2.37-2.88 (m, 1H), | |
| | 1-yl]-2- | 3-amine | | 3.06-3.13 (m, 1H), 3.25-3.33 (m | |
| | methoxy-phenyl]- | (Method 7) | | partially hidden by H2O, | |
| | 4-imidazo[1,2-a]pyridin- | | | 1H), 3.34-3.46 (m partially | |
| | 3-yl- | | | hidden by H2O, 1H), | |
| | pyrimidin-2-amine | | | 3.46-3.54 (m, 1H), 3.75 (s, 3H), | |
| | | | | 6.16 (dd, 1H), 6.28 (s, 1H), | |
| | | | | 6.82 (bs, 1H), 7.15 (d, 1H), | |
| | | | | 7.47 (dd, 1H), 7.74 (d, | |
| | | | | 1H), 8.42 (s, 1H), 8.72 (s, | |
| | | | | 2H), 9.49 (bs, 1H) | |
| 3.12 | 5-chloro-4- | 2-methoxy-4-[2-[4- | 538 | (DMSOd6 + TFAd) | 59 |
| | imidazo[1,2-a]pyridin- | (2-methoxyethyl)piperazin- | | 3.34 (s, 3H), 3.45-3.51 (m, 2H), | |
| | 3-yl-N-[2- | 1-yl]ethoxy]aniline | | 3.68 (bs, 8H), | |
| | methoxy-4-[2-[4- | (Method 10) | | 3.69-3.76 (m, 4H), 3.82 (s, 3H), | |
| | (2-methoxyethyl)piperazin- | | | 4.41-4.49 (m, 2H), | |
| | 1-yl]ethoxy]phenyl]pyrimidin- | | | 6.67 (dd, 1H), 6.81 (d, 1H), | |
| | 2-amine | | | 7.47 (d, 1H), 7.50 (bs, 1H), | |
| | | | | 8.05-8.14 (m, 2H), 8.65 (s, | |
| | | | | 1H), 9.18 (s, 1H), 9.85 (bs, | |
| | | | | 1H) | |
| 3.13 | 5-chloro-N-[5- | (3R)-1-(3-amino-4- | 464 | 1.69-1.80 (m, 1H), 2.13 (s, | 84 |
| | [(3R)-3-dimethylaminopyrrolidin- | methoxyphenyl)- | | 6H), 2.67-2.77 (m, 1H), | |
| | 1- | N,N-dimethylpyrrolidin- | | 2.90-2.97 (m, 1H), | |
| | yl]-2-methoxy- | 3-amine | | 3.10-3.18 (m, 1H), | |
| | phenyl]-4- | (Method 8) | | 3.20-3.27 (m, 1H), 3.28-3.35 (m | |
| | imidazo[1,2-a]pyridin- | | | partially hidden by H2O, | |
| | 3-yl- | | | 1H), 3.70 (s, 3H), 6.39 (dd, | |
| | pyrimidin-2-amine | | | 1H), 6.84-6.92 (m, 2H), | |
| | | | | 7.00 (d, 1H), 7.49 (dd, | |
| | | | | 1H), 7.76 (d, 1H), 8.52 (s, | |
| | | | | 1H), 8.70 (s, 1H), 8.84 (bs, | |
| | | | | 1H), 9.57 (bs, 1H) | |
| 3.14 | 5-chloro-N-[5- | (3S)-1-(3-amino-4- | 448 | 1.69-1.81 (m, 1H), 2.07 (s, | 24 |
| | [(3S)-3- | methylphenyl)- | | 3H), 2.08-2.14 (m, 1H), | |
| | dimethylaminopyrrolidin- | N,N- | | 2.15 (s, 6H), 2.68-2.77 (m, | |
| | 1-yl]-2- | dimethylpyrrolidin- | | 1H), 2.95-3.03 (m, 1H), | |
| | methyl-phenyl]-4- | 3-amine (Method | | 3.15-3.24 (m, 1H), | |
| | imidazo[1,2-a]pyridin- | 11) | | 3.28-3.34 (m partially hidden by | |
| | 3-yl- | | | H2O, 1H), 3.36-3.42 (m | |
| | pyrimidin-2-amine | | | partially hidden by H2O, | |
| | | | | 1H), 6.46 (dd, 1H), | |
| | | | | 6.58 (d, 1H), 7.72 (bs, 1H), | |
| | | | | 7.11 (d, 1H), 7.47 (ddd, 1H), | |
| | | | | 7.74 (d, 1H), 8.48 (s, 1H), | |
| | | | | 8.77 (s, 1H), 9.17 (s, 1H), | |
| | | | | 9.50 (bs, 1H) | |
| 3.15 | 5-chloro-N-[4-(1,1- | 4-(1,1-dioxo-1,4- | 485 | 3.13-3.22 (m, 4H), 3.78 (s, | 21 |
| | dioxo-1,4- | thiazinan-4-yl)-2- | | 3H), 3.79-3.88 (m, 4H), | |
| | thiazinan-4-yl)-2- | methoxyaniline | | 6.63 (dd, 1H), 6.78 (d, | |
| | methoxy-phenyl]- | (Method 7) | | 1H), 6.92 (bs, 1H), 7.33 (d, | |
| | 4-imidazo[1,2- | | | 1H), 7.49 (dd, 1H), | |
| | a]pyridin-3-yl- | | | 7.76 (d, 1H), 8.47 (s, 1H), | |
| | pyrimidin-2-amine | | | 8.71 (s, 1H), 8.82 (s, 1H), | |
| | | | | 9.57 (bs, 1H) | |
| 3.16 | 5-chloro-N-[4- | (3S)-1-(4-amino-3- | 464 | 1.78-1.91 (m, 1H), | 56 |
| | [(3S)-3- | methoxyphenyl)- | | 2.14-2.22 (m, 1H), 2.23 (s, 6H), | |
| | dimethylaminopyrrolidin- | N,N-dimethylpyrrolidin- | | 2.78-2.88 (m, 1H), | |
| | 1-yl]-2- | 3-amine | | 3.10 (dd, 1H), 3.26-3.32 (m | |
| | methoxy-phenyl]- | (Method 7) | | partially hidden by H2O, | |
| | 4-imidazo[1,2-a]pyridin- | | | 1H), 3.42 (ddd, 1H), | |
| | 3-yl- | | | 3.50 (ddd, 1H), 3.75 (s, 3H), | |
| | pyrimidin-2-amine | | | 6.15 (dd, 1H), 6.28 (d, | |
| | | | | 1H), 6.82 (bs, 1H), 7.15 (d, | |
| | | | | 1H), 7.47 (dd, 1H), | |
| | | | | 7.74 (d, 1H), 8.42 (dm, 1H), | |
| | | | | 8.71 (bs, 2H), 9.48 (bs, | |
| | | | | 1H) | |
| 3.17 | 5-chloro-N-[5- | (3S)-1-(3-amino-4- | 464 | 1.69-1.79 (m, 1H), | 51 |
| | [(3S)-3- | methoxyphenyl)- | | 2.04-2.11 (m, 1H), 2.13 (s, 6H), | |
| | dimethylaminopyrrolidin- | N,N-dimethylpyrrolidin- | | 2.68-2.73 (m, 1H), | |
| | 1-yl]-2- | 3-amine | | 2.93 (dd, 1H), 3.14 (ddd, 1H), | |
| | methoxy-phenyl]- | (Method 8) | | 3.24 (ddd, 1H), | |
| | 4-imidazo[1,2-a]pyridin- | | | 3.29-3.33 (m, partially hidden by | |
| | 3-yl- | | | H2O, 1H), 3.74 (s, 3H), | |
| | pyrimidin-2-amine | | | 6.39 (dd, 1H), | |
| | | | | 6.84-6.92 (m, 2H), 7.00 (d, 1H), | |
| | | | | 7.49 (ddd, 1H), 7.76 (ddd, 1H), | |
| | | | | 8.52 (s, 1H), 8.70 (s, 1H), | |
| | | | | 8.83 (s, 1H), 9.57 (bs, 1H) | |
| 3.18^{b} | (3R)-1-[3-[(5- | (3R)-1-(3-amino-4- | 437 | 1.79-1.87 (m, 1H), | 28 |
| | chloro-4- | methoxyphenyl)pyrrolidin- | | 1.95-2.04 (m, 1H), 2.97 (dd, | |
| | imidazo[1,2-a]pyridin- | 3-ol | | 1H), 3.16 (ddd, 1H), | |
| | 3-yl- | (Method 8) | | 3.24 (dd, 1H), 3.69 (s, 3H), | |
| | pyrimidin-2- | | | 4.2-4.38 (m, 1H), 4.90 (d, 1H), | |
| | yl)amino]-4- | | | 6.36 (dd, 1H), 6.83 (d, | |
| | methoxy-phenyl]pyrrolidin- | | | 1H), 6.91 (dd, 1H), | |
| | 3-ol | | | 7.00 (d, 1H), 7.48 (ddd, 1H), | |
| | | | | 7.76 (d, 1H), 8.52 (s, 1H), | |
| | | | | 8.71 (s, 1H), 8.86 (s, 1H), | |
| | | | | 9.60 (bs, 1H) | |
| 3.19^{b} | 5-chloro-4- | 2-methoxy-5- | 437 | 2.92-3.01 (m, 4H), | 49 |
| | imidazo[1,2-a]pyridin- | morpholin-4- | | 3.64-3.72 (m, 4H), 3.74 (s, 3H), | |
| | 3-yl-N-(2- | ylaniline (Method | | 6.78 (dd, 1H), 6.95 (dd, | |
| | methoxy-5- | 8) | | 1H), 7.03 (d, 1H), 7.29 (d, | |
| | morpholino- | | | 1H), 7.51 (ddd, 1H), | |
| | phenyl)pyrimidin- | | | 7.77 (d, 1H), 8.54 (s, 1H), | |
| | 2-amine | | | 8.71 (s, 1H), 8.85 (s, 1H), | |
| | | | | 9.56 (bs, 1H) | |
| 3.20^{b} | 5-chloro-4- | 2-methoxy-5-(4- | 465 | 1.43-1.54 (m, 2H), | 64 |
| | imidazo[1,2-a]pyridin- | methoxypiperidin- | | 1.82-1.93 (m, 2H), 2.69-2.77 m, | |
| | 3-yl-N-[2- | 1-yl)aniline | | 2H), 3.24 (s, 3H), | |
| | methoxy-5-(4- | (Method 8) | | 3.25-3.35 (m partially hidden by | |
| | methoxy-1- | | | H2O, 3H), 3.73 (s, 3H), | |
| | piperidyl)phenyl]pyrimidin- | | | 6.78 (dd, 1H), 6.94 (dd, | |
| | 2-amine | | | 1H), 7.00 (d, 1H), 7.29 (s, | |
| | | | | 1H), 7.50 (ddd, 1H), | |
| | | | | 7.77 (d, 1H), 8.54 (s, 1H), | |
| | | | | 8.70 (s, 1H), 8.81 (s, 1H), | |
| | | | | 9.55 (bs, 1H) | |
| 3.21 | 1-[4-[4-[(5-chloro- | 1-[4-(4-amino-3- | 477 | 1.47-1.58 (m, 1H), | 56 |
| | 4-imidazo[1,2-a]pyridin- | methoxyphenyl)piperidin- | | 1.62-1.72 (m, 1H), | |
| | 3-yl- | 1- | | 1.78-1.88 (m, 2H), 2.05 (s, 3H), | |
| | pyrimidin-2- | yl]ethanone | | 2.55-2.63 (m, 1H), | |
| | yl)amino]-3- | (Method 12) | | 2.76-2.84 (m, 1H), | |
| | methoxy-phenyl]- | | | 3.10-3.18 (m, 1H), 3.80 (s, 3H), | |
| | 1-piperidyl]ethanone | | | 3.92-3.99 (m, 1H), | |
| | | | | 4.53-4.61 (m, 1H), 6.85 (dd, | |
| | | | | 1H), 6.92 (dd, 1H), | |
| | | | | 7.02 (d, 1H), 7.48 (d, 1H), | |
| | | | | 7.51 (ddd, 1H), 7.77 (d, 1H), | |
| | | | | 8.50 (s, 1H), 8.73 (s, 1H), | |
| | | | | 8.88 (s, 1H), 9.60 (bs, 1H) | |
| 3.22^{b} | 5-chloro-4- | 2-methoxy-4-(4- | 514 | 2.95 (s, 3H), 3.26-3.33 (m, | 71 |
| | imidazo[1,2-a]pyridin- | methyl sulfonylpiperazin- | | 8H), 3.77 (s, 3H), 6.58 (dd, | |
| | 3-yl-N-[2- | 1- | | 1H), 6.76 (d, 1H), 6.90 (bs, | |
| | methoxy-4-(4- | yl)aniline (Method | | 1H), 7.31 (d, 1H), | |
| | methylsulfonylpiperazin- | 7) | | 7.50 (ddd, 1H), 7.76 (d, 1H), | |
| | 1-yl)phenyl]pyrimidin- | | | 8.46 (s. 1H), 8.72 (s, 1H), | |
| | 2-amine | | | 8.82 (s, 1H), 9.56 (bs, 1H) | |
| 3.23^{b} | N-[(3R)-1-[4-[(5- | N-[(3R)-1-(4- | 478 | 1.83 (s, 3H), 1.87-1.96 (m, | 70 |
| | chloro-4-imidazo[1, | amino-3- | | 1H), 2.18-2.27 (m, 1H), | |
| | 2-a]pyridin-3-yl- | methoxyphenyl)pyrrolidin- | | 3.11 (dd, 1H), | |
| | pyrimidin-2-yl)amino]- | 3-yl]acetamide | | 3.33-3.39 (m partially hidden by | |
| | 3-methoxy- | (Method | | H2O, 1H), 3.40-3.48 (m, | |
| | phenyl]pyrrolidin- | 7) | | 1H), 3.55 (dd, 1H), 3.75 (s, | |
| | 3-yl]acetamide | | | 3H), 4.36-4.44 (m, 1H), | |
| | | | | 6.15 (dd, 1H), 6.26 (d, | |
| | | | | 1H), 6.84 (bs, 1H), 7.17 (d, | |
| | | | | 1H), 7.47 (dd, 1H), | |
| | | | | 7.75 (d, 1H), 8.18 (d, 1H), | |
| | | | | 8.42 (s, 1H), 8.71 (s, 1H), | |
| | | | | 8.72 (s, 1H), 9.51 (bs, 1H) | |
| 3.24^{b} | 4-[4-[(5-chloro-4- | 4-(4-amino-3- | 435 | 2.34-2.51 (m, 2H), 3.33 (bs | 58 |
| | imidazo[1,2-a]pyridin- | methoxyphenyl)pyrrolidin- | | partially hidden by H2O, | |
| | 3-yl- | 2-one | | 1H), 3.65 (bs, 2H), 3.81 (s, | |
| | pyrimidin-2-yl)amino]- | (Method 13) | | 3H), 6.92 (s, 1H), 6.93 (s, | |
| | 3-methoxy- | | | 1H), 7.11 (s, 1H), | |
| | phenyl]pyrrolidin- | | | 6.46-6.56 (m, 2H), | |
| | 2-one | | | 7.70-7.80 (m, 2H), 8.51 (s, 1H), | |
| | | | | 8.73 (s, 1H), 8.92 (s, 1H), | |
| | | | | 9.59 (bs, 1H) | |
| 3.25^{b} | 1-[2-[4-[(5-chloro- | 1-[2-(4-amino-3- | 477 | 1.27-1.97 (m, 4H), | 16 |
| | 4-imidazo[1,2-a]pyridin- | methoxyphenyl)piperidin- | | 2.03 (bs, 1.05H), 2.17 (bs, | |
| | 3-yl- | 1-yl]ethanone | | 1.95H), 2.37-2.46 (m, 2H), | |
| | pyrimidin-2-yl)amino]- | (Method | | 2.52-2.63 (m, 0.35H), | |
| | 3-methoxy- | 14) | | 2.97-3.10 (m, 0.65H), | |
| | phenyl]-1- | | | 3.75 (bs, 0.65H), 3.78 (s, 3H), | |
| | piperidyl]ethanone | | | 4.43 (bs, 0.35H), 5.19 (bs, | |
| | | | | 0.35H), 5.78 (bs, 0.65H), | |
| | | | | 6.79 (d, 1H), 6.90 (s, 1H), | |
| | | | | 6.94 (dd, 1H), 7.50 (ddd, | |
| | | | | 1H), 7.55 (bs, 0.65H), | |
| | | | | 7.62 (bs, 0.35H), 7.77 (d, 1H), | |
| | | | | 8.52 (s, 1H), 8.73 (s, 1H), | |
| | | | | 8.95 (s, 1H), 9.60 (bs, 1H) | |
| 3.26^{b} | 1-[4-[4-[(5-chloro- | 1-[4-(4-amino-3- | 496 | 2.06 (s, 3H), 3.23 (bs, 2H), | 54 |
| | 4-imidazo[1,2-a]pyridin- | fluoro-5- | | 3.28 (bs, 2H), 3.61 (bs, | |
| | 3-yl- | methoxyphenyl)piperazin- | | 4H), 3.76 (s, 3H), 6.50 (dd, | |
| | pyrimidin-2-yl)amino]- | 1-yl]ethanone | | 1H), 6.52 (s, 1H), 6.84 (bs, | |
| | 3-fluoro-5- | (Method | | 1H), 7.47 (dd, 1H), | |
| | methoxy-phenyl]piperazin- | 15) | | 7.73 (d, 1H), 8.43 (s, 1H), | |
| | 1-yl]ethanone | | | 8.65 (s, 1H), 8.72 (s, 1H), | |
| | | | | 9.44 (bs, 1H) | |
| 3.27 | 4-[4-[(5-chloro-4- | 4-(4-amino-3- | 478 | 1.75-1.84 (m, 1H), | 30 |
| | imidazo[1,2-a]pyridin- | methoxyphenyl)-N- | | 2.47-2.56 (m partially hidden by | |
| | 3-yl- | methylpyrrolidine- | | DMSOd5, 2H), 2.64 (d, | |
| | pyrimidin-2- | 2-carboxamide | | 3H), 2.69-2.75 (m, 1H), | |
| | yl)amino]-3- | (Method 16) | | 3.17-3.26 (m, 1H), | |
| | methoxy-phenyl]- | | | 3.70-3.76 (m, 1H), 3.78 (s, 3H), | |
| | N-methyl- | | | 6.84 (dd, 1H), 6.90 (dd, | |
| | pyrrolidine-2- | | | 1H), 6.99 (d, 1H), 6.46 (d, | |
| | carboxamide | | | 1H), 6.50 (ddd, 1H), | |
| | | | | 7.77 (d, 1H), 7.98 (q, 1H), | |
| | | | | 8.50 (s, 1H), 8.73 (s, 1H), | |
| | | | | 8.89 (s, 1H), 9.57 (bs, 1H) | |
| 3.28 | 1-[4-[4-[(5-chloro- | 1-[4-(4-amino-3- | 492 | 1.81-1.87 (m, 1H), | 48 |
| | 4-imidazo[1,2-a]pyridin- | methoxyphenyl)- | | 1.87-1.95 (m, 1H), 1.88 (s, | |
| | 3-yl- | 1,4-diazepan-1- | | 1.5H), 2.01 (s, 1.5H), | |
| | pyrimidin-2-yl)amino]- | yl]ethanone | | 3.33-3.42 (m, 2H), | |
| | 3-methoxy- | (Method 7) | | 3.43-3.68 (m, 5H), 3.69-3.74 (m, | |
| | phenyl]-1,4- | | | 1H), 3.75 (s, 3H), 6.36 (d, | |
| | diazepan-1- | | | 1H), 6.45 (s, 1H), 6.84 (bs, | |
| | yl]ethanone | | | 1H), 7.15 (dd, 1H), | |
| | | | | 7.48 (dd, 1H), 7.74 (d, 1H), | |
| | | | | 8.43 (s, 1H), 8.72 (bs, 2H) | |
| | | | | 9.53 (bs, 1H) | |
| 3.29 | 1-[3-[4-[(5-chloro- | 1-[3-(4-amino-3- | 477 | 1.37-1.48 (m, 0.5H), | 32 |
| | 4-imidazo[1,2-a]pyridin- | methoxyphenyl)piperidin- | | 1.51-1.61 (m, 0.5H), | |
| | 3-yl- | 1-yl]ethanone | | 1.71-1.84 (m, 2H), 1.92-2.00 (m, | |
| | pyrimidin-2- | (Method | | 1H), 2.05 (s, 3H), | |
| | yl)amino]-3- | 17) | | 2.53-2.63 (m, 1H), | |
| | methoxy-phenyl]- | | | 2.67-2.79 (m, 1H), 3.07-3.15 (m, | |
| | 1-piperidyl]ethanone | | | 0.5H), 3.15-3.22 (m, | |
| | | | | 0.5H), 3.80 (s, 1.5H), | |
| | | | | 3.82 (s, 1.5H), 3.82-3.89 (m, | |
| | | | | 1H), 4.41-4.51 (m, 1H), | |
| | | | | 6.85-6.90 (m, 0.5H), | |
| | | | | 6.90-6.98 (m, 1.5H), 7.03 (s, | |
| | | | | 0.5H), 7.10 (s, 0.5H), | |
| | | | | 7.47-7.55 (m, 2H), 7.76 (d, 1H), | |
| | | | | 8.51 (s, 1H), 8.73 (s, 1H), | |
| | | | | 8.91 (s, 0.5H), 8.92 (s, | |
| | | | | 0.5H), 9.59 (bs, 1H) | |
| 3.30 | 1-[4-[4-[(5-chloro- | 1-[4-(4-amino-3- | 462 | 2.07 (s, 3H) 2.16 (s, 3H), | 38 |
| | 4-imidazo[1,2-a]pyridin- | methylphenyl)piperazin- | | 3.10-3.16 (m, 2H), | |
| | 3-yl- | 1-yl]ethanone | | 3.16-3.22 (m, 2H), | |
| | pyrimidin-2-yl)amino]- | (described | | 3.57-3.66 (m, 4H), 6.67 (bs, 1H), | |
| | 3-methyl- | in EP 257864) | | 6.88 (dd, 1H), 6.94 (d, | |
| | phenyl]piperazin-1- | | | 1H), 7.21 (d, 1H), | |
| | yl]ethanone | | | 7.48 (ddd, 1H), 7.74 (d, 1H), | |
| | | | | 8.45 (s, 1H), 8.75 (s, 1H), | |
| | | | | 9.08 (s, 1H), 9.41 (bs, 1H) | |
| 3.31^{b} | 1-[4-[4-[(5-chloro- | 1-[4-(4-amino-3- | 506 | 1.34 (bs, 6H), 2.09 (s, 3H), | 42 |
| | 4-imidazo[1,2-a]pyridin- | methoxyphenyl)- | | 2.84 (bs, 2H), | |
| | 3-yl- | 2,6- | | 3.55-3.65 (m, 2H), 3.78 (s, 3H), | |
| | pyrimidin-2-yl)amino]- | dimethylpiperazin- | | 4.16 (bs, 1H), 4.52 (bs, 1H), | |
| | 3-methoxy- | 1-yl]ethanone | | 6.60 (dd, 1H), 6.70 (d, | |
| | phenyl]-2,6- | (Method 7) | | 1H), 6.87 (bs, 1H), 7.29 (s, | |
| | dimethyl-piperazin- | | | 1H), 7.47 (dd, 1H), | |
| | 1-yl]ethanone | | | 7.75 (d, 1H), 8.45 (s, 1H), | |
| | | | | 8.74 (s, 1H), 8.83 (s, 1H), | |
| | | | | 9.53 (bs, 1H) | |
| 3.32 | 1-[4-[4-[(5-chloro- | 1-[4-(4-amino-2,3- | 476 | 2.07 (s, 3H), 2.10 (s, 3H), | 28 |
| | 4-imidazo[1,2-a]pyridin- | dimethylphenyl)piperazin- | | 2.26 (s, 3H), 2.75-2.83 (m, | |
| | 3-yl- | 1-yl]ethanone | | 2H), 2.83-2.91 (m, 2H), | |
| | pyrimidin-2-yl)amino]- | (Method | | 3.62 (bs, 4H), 6.72 (bs, | |
| | 2,3- | 18) | | 1H), 7.00 (d, 1H), 7.17 (d, | |
| | dimethyl- | | | 1H), 7.48 (ddd, 1H), | |
| | phenyl]piperazin-1- | | | 7.74 (d, 1H), 8.47 (s, 1H), | |
| | yl]ethanone | | | 8.76 (s, 1H), 9.24 (s, 1H), | |
| | | | | 9.34 (bs, 1H) | |
| 3.33 | 1-[4-[4-[(5-chloro- | 1-[4-(4-amino-2- | 480 | 2.05 (s, 3H), 2.17 (s, 3H), | 23 |
| | 4-imidazo[1,2-a]pyridin- | fluoro-5-methylphenyl)piperazin- | | 2.95-3.01 (m, 2H), | |
| | 3-yl- | 1- | | 3.01-3.08 (m, 2H), | |
| | pyrimidin-2-yl)amino]- | yl]ethanone | | 3.57-3.67 (m, 4H), 6.85 (bs, 1H), | |
| | 2-fluoro-5- | (Method 19) | | 6.97 (d, 1H), 7.28 (d, 1H), | |
| | methyl-phenyl]piperazin- | | | 7.51 (ddd, 1H), 7.77 (d, | |
| | 1-yl]ethanone | | | 1H), 8.51 (s, 1H), 8.76 (s, | |
| | | | | 1H), 9.21 (s, 1H), 9.52 (s, | |
| | | | | 1H) | |
| 3.34 | 1-[4-[4-[(5-chloro- | 1-[4-(4-amino-3- | 506 | 1.13 (d, 1H), 2.06 (s, 3H), | 35 |
| | 4-imidazo[1,2-a]pyridin- | propan-2-yloxyphenyl)piperazin- | | 3.10-3.16 (m, 2H), | |
| | 3-yl- | 1- | | 3.16-3.22 (m, 2H), | |
| | pyrimidin-2-yl)amino]- | yl]ethanone | | 3.57-3.66 (m, 4H), 4.54-4.64 (m, | |
| | 3- | (Method 20) | | 1H), 6.57 (dd, 1H), | |
| | isopropoxy- | | | 6.72 (d, 1H), 6.85 (bs, 1H), | |
| | phenyl]piperazin-1- | | | 7.33 (d, 1H), 7.49 (ddd, 1H), | |
| | yl]ethanone | | | 7.75 (d, 1H), 8.47 (s, 1H), | |
| | | | | 8.67 (s, 1H), 8.72 (s, 1H), | |
| | | | | 9.52 (bs, 1H) | |
| 3.35^{b} | 1-[(2R,5S)-4-[4- | 1-[(2S,5R)-4-(4- | 506 | 0.89 (d, 1.5H), 0.98 (d, | 41 |
| | [(5-chloro-4- | amino-3-methoxyphenyl)- | | 1.5H), 1.20 (d, 1.5H), | |
| | imidazo[1,2- | 2,5- | | 1.32 (d, 1.5H), 2.02 (s, | |
| | a]pyridin-3-yl- | dimethyl piperazin- | | 1.5H). 2.12 (s, 1.5H), | |
| | pyrimidin-2-yl)amino]- | 1-yl]ethanone | | 3.15 (ddd, 1H), 3.21-3.30 (m, | |
| | 3-methoxy- | (Method 7) | | 1.5H), 3.54-3.61 (m, | |
| | phenyl]-2,5- | | | 0.5H), 3.65-3.71 (0.5H), | |
| | dimethyl- | | | 3.76 (s, 3H), 4.15 (bs, | |
| | piperazin-1-yl]ethanone | | | 0.5H), 4.16-4.24 (m, 1H), | |
| | | | | 4.75 (s, 1H), 6.50 (dd, 1H), | |
| | | | | 6.67 (s, 1H), 6.85 (bs, 1H), | |
| | | | | 7.24 (d, 1H), 7.47 (dd, | |
| | | | | 1H), 7.74 (d, 1H), 8.45 (s, | |
| | | | | 1H), 8.72 (s, 1H), 8.79 (s, | |
| | | | | 1H), 9.51 (bs, 1H) | |
| 3.36^{b} | 1-[4-[4-[(5-chloro- | 1-[4-(4-amino-3- | 492 | 1.21 (d, 1.5H), 1.24 (d, | 47 |
| | 4-imidazo[1,2-a]pyridin- | methoxyphenyl)-2- | | 1.5H), 2.05 (s, 1.5H), | |
| | 3-yl- | methylpiperazin-1- | | 2.08 (s, 1.5H), 2.51-2.53 (m | |
| | pyrimidin-2-yl)amino]- | yl]ethanone | | partially hidden by | |
| | 3-methoxy- | (Method 21) | | DMSOd5, 0.5H), | |
| | phenyl]-2-methyl- | | | 2.55-2.67 (m, 0.5H), | |
| | piperazin-1-yl]ethanone | | | 2.72-2.80 (m, 0.5H), 2.80-2.88 (m, | |
| | | | | 0.5H), 2.88-2.95 (m, | |
| | | | | 0.5H), 3.44-3.54 (m, | |
| | | | | 0.5H), 3.54-3.62 (m, 1H), | |
| | | | | 3.65-3.76 (m, 1.5H), | |
| | | | | 3.77 (s, 3H), 4.19 (bs, 0.5H), | |
| | | | | 4.26-4.36 (m, 0.5H), | |
| | | | | 4.67 (bs, 0.5H), 6.54 (d, 1H), | |
| | | | | 6.70 (s, 1H), 6.88 (bs, 1H), | |
| | | | | 7.28 (d, 1H), 7.48 (ddd, | |
| | | | | 1H), 7.75 (d, 1H), 8.45 (s, | |
| | | | | 1H), 8.72 (s, 1H), 8.81 (s, | |
| | | | | 1H), 9.53 (bs, 1H) | |
| 3.37^{b} | 1-[4-[3-[(5-chloro- | 1-[4-(3-amino-2- | 462 | 2.05 (s, 3H), 2.19 (s, 3H), | 22 |
| | 4-imidazo[1,2-a]pyridin- | methylphenyl)piperazin- | | 2.74-2.82 (m, 2H), | |
| | 3-yl- | 1-yl]ethanone | | 2.82-2.90 (m, 2H), | |
| | pyrimidin-2-yl)amino]- | (Method 22) | | 3.50-3.64 (m, 4H), 6.79 (dd, 1H), | |
| | 2-methyl- | | | 7.02 (d, 1H), 7.16 (d, 1H), | |
| | phenyl]piperazin-1- | | | 7.27 (dd, 1H), 7.47 (ddd, | |
| | yl]ethanone | | | 1H), 7.75 (d, 1H), 8.50 (s, | |
| | | | | 1H), 8.77 (s, 1H), 9.28 (s, | |
| | | | | 1H), 9.39 (bs, 1H), | |
| 3.38 | 1-[4-[4-[(5-chloro- | 1-[4-(4-amino-3- | 508 | 2.06 (s, 1.2H), 2.10 (s, | 10 |
| | 4-imidazo[1,2-a]pyridin- | methoxyphenyl)-2- | | 1.8H), 2.51-2.54 (m | |
| | 3-yl- | (hydroxymethyl)piperazin- | | partially hidden by | |
| | pyrimidin-2-yl)amino]- | 1-yl]ethanone | | DMSOd5, 0.4H), | |
| | 3-methoxy- | (Method | | 2.55-2.63 (m, 0.6H), | |
| | phenyl]-2- | 23) | | 2.72-2.82 (m, 0.8H), 2.82-2.87 (m, | |
| | (hydroxymethyl)piperazin- | | | 0.6H), 2.90-2.99 (m, | |
| | 1-yl]ethanone | | | 0.6H), 3.35-3.43 (m, 1H), | |
| | | | | 3.64-3.75 (m, 3H), 3.76 (s, | |
| | | | | 3H), 3.82-3.88 (m, 0.4H), | |
| | | | | 3.96-4.03 (m, 0.6H), | |
| | | | | 4.31-4.36 (m, 0.6H), | |
| | | | | 4.47-4.53 (m, 0.4H), 4.90 (t, 0.4H), | |
| | | | | 4.99 (t, 0.6H), 6.55 (d, | |
| | | | | 1H), 6.70 (s, 1H), 6.88 (bs, | |
| | | | | 1H), 7.28 (d, 1H), | |
| | | | | 7.48 (dd, 1H), 7.75 (d, 1H), | |
| | | | | 8.45 (s, 1H), 8.73 (s, 1H), | |
| | | | | 8.81 (s, 1H), 9.53 (bs, 1H) | |
| 3.39^{b} | 2-[4-[(5-chloro-4- | 2-(4-amino-3- | 493 | 1.36 (d, 3H), 3.11-3.20 (m, | 44 |
| | imidazo[1,2-a]pyridin- | methoxyphenyl)-1- | | 1H), 3.23-3.32 (m partially | |
| | 3-yl- | morpholin-4-ylpropan- | | hidden by H2O, 1H), | |
| | pyrimidin-2-yl)amino]- | 1-one | | 3.40-3.63 (m, 6H), 3.78 (s, 3H), | |
| | 3-methoxy- | (Method 24) | | 4.14 (q, 1H), 6.86 (dd, | |
| | phenyl]-1- | | | 1H), 6.93 (dd, 1H), | |
| | morpholino- | | | 7.05 (d, 1H), 7.48-7.56 (m, 2H), | |
| | propan-1-one | | | 7.77 (d, 1H), 8.52 (s, 1H), | |
| | | | | 8.77 (s, 1H), 8.92 (s, 1H), | |
| | | | | 9.59 (bs, 1H) | |
| 3.40^{b} | 1-[(6S)-4-[4-[(5- | 1-[(6S)-4-(4- | 508 | 1.86 (s, 1.35H), | 30 |
| | chloro-4-imidazo[1, | amino-3- | | 2.06 (1.65H), dd, 2.45 (dd, | |
| | 2-a]pyridin-3-yl- | methoxyphenyl)-6- | | 0.45H), 3.22-3.46 (m, 2H), | |
| | pyrimidin-2-yl)amino]- | hydroxy-1,4- | | 3.48-3.62 (m, 1.55H), | |
| | 3-methoxy- | diazepan-1-yl]ethanone | | 3.66-3.83 (m, 3H), 3.74 (s, | |
| | phenyl]-6-hydroxy- | (Method 25) | | 3H), 3.84-3.93 (m, 1H), | |
| | 1,4-diazepan-1- | | | 3.95-4.02 (m, 0.55H), | |
| | yl]ethanone | | | 4.05 (dd, 0.45H), 5.03 (d, | |
| | | | | 0.45H), 5.14 (d, 0.55H), | |
| | | | | 6.38-6.46 (m, 1H), 6.52 (d, | |
| | | | | 0.55H), 6.55 (d, 0.45H), | |
| | | | | 6.79-6.87 (m, 1H), | |
| | | | | 7.19 (dd, 1H), 7.46 (ddd, 1H), | |
| | | | | 7.72 (d, 1H), 8.41 (s, 1H), | |
| | | | | 8.53 (s, 1H), 8.70 (s, 1H), | |
| | | | | 9.54 (bs, 1H) | |
| 3.41^{b} | 2-[4-[(5-chloro-4- | 2-(4-amino-3- | 536 | 1.34 (d, 3H), 2.04 (bs, 1H), | 52 |
| | imidazo[1,2-a]pyridin- | methoxyphenyl)-1- | | 2.31 (bs, 4H), 2.39 (bs, | |
| | 3-yl- | [4-(2-hydroxyethyl)piperazin- | | 1H), 3.29-3.63 (m, 6H), | |
| | pyrimidin-2-yl)amino]- | 1-yl]propan- | | 3.80 (s, 3H), 4.13 (q, 1H), | |
| | 3-methoxy- | 1-one | | 4.23 (t, 1H), 6.88 (d, 1H), | |
| | phenyl]-1-[4-(2- | (Method 24) | | 6.93 (dd, 1H), 7.04 (s, 1H), | |
| | hydroxyethyl)piperazin- | | | 7.50 (dd, 1H), 7.58 (d, | |
| | 1-yl]propan- | | | 1H), 7.76 (d, 1H), 8.51 (s, | |
| | 1-one | | | 1H), 8.72 (s, 1H), 8.74 (s, | |
| | | | | 1H), 9.59 (s, 0.5H), | |
| | | | | 9.61 (s, 0.5H) | |
| 3.42^{b} | 2-[4-[(5-chloro-4- | tert-butyl 4-[2-(4- | 492 | 1.34 (d, 3H), 2.16-2.29 (m, | 28 |
| | imidazo[1,2-a]pyridin- | amino-3-methoxyphenyl)propanoyl]piperazine- | | 2H), 2.56-2.67 (m, 2H), | |
| | 3-yl- | 1- | | 3.20-3.33 (m, 2H), | |
| | pyrimidin-2-yl)amino]- | carboxylate | | 3.36-3.54 (m, 2H), 3.78 (s, 3H), | |
| | 3-methoxy- | (Method 24)^{d} | | 4.12 (q, 1H), 6.86 (dd, | |
| | phenyl]-1- | | | 1H), 6.93 (dd, 1H), 7.04 (s, | |
| | piperazin-1-yl- | | | 1H), 7.47-7.55 (m, 2H), | |
| | propan-1-one | | | 7.77 (d, 1H), 8.52 (s, 1H), | |
| | | | | 8.73 (s, 1H), 8.91 (s, 1H), | |
| | | | | 9.60 (bs, 1H) | |
| 3.43^{a,b} | 5-chloro-4- | 2-methoxy-4- | 437 | 3.14-3.20 (m, 4H), 3.76 (s, | 63 |
| | imidazo[1,2-a]pyridin- | morpholin-4- | | 3H), 3.76-3.81 (m, 4H), | |
| | 3-yl-N-(2- | ylaniline (Method | | 6.54 (dd, 1H), 6.71 (d, | |
| | methoxy-4- | 7) | | 1H), 6.89 (bs, 1H), 6.29 (d, | |
| | morpholino- | | | 1H), 7.50 (ddd, 1H), | |
| | phenyl)pyrimidin- | | | 7.75 (d, 1H), 8.45 (s, 1H), | |
| | 2-amine | | | 8.72 (s, 1H), 8.80 (s, 1H), | |
| | | | | 9.54 (bs, 1H) | |
| 3.44^{a,b} | 5-chloro-4- | 2-methoxy-4- | 421 | 1.96-2.04 (m, 4H), | 50 |
| | imidazo[1,2-a]pyridin- | pyrrolidin-1- | | 3.26-3.33 (m, 4H), 3.74 (s, 3H), | |
| | 3-yl-N-(2- | ylaniline (Method | | 6.16 (dd, 1H), 6.27 (d, | |
| | methoxy-4- | 7) | | 1H), 6.81 (bs, 1H), 7.15 (d, | |
| | pyrrolidin-1-yl- | | | 1H), 7.48 (dd, 1H), | |
| | phenyl)pyrimidin- | | | 7.74 (d, 1H), 8.42 (s, 1H), | |
| | 2-amine | | | 8.71 (s, 1H), 8.72 (s, 1H), | |
| | | | | 9.50 (bs, 1H) | |
| 3.45^{a} | 2-[4-[4-[(5-chloro- | 2-[4-(4-amino-3- | 480 | 2.46 (t, 2H), 2.56-2.63 (m, | 51 |
| | 4-imidazo[1,2-a]pyridin- | methoxyphenyl)piperazin- | | 4H), 3.16-3.23 (m, 4H), | |
| | 3-yl- | 1-yl | | 3.52-3.59 (m, 2H), 3.75 (s, | |
| | pyrimidin-2-yl)amino]- | ethanol (Method 7) | | 3H), 4.45 (bs, 1H), | |
| | 3-methoxy- | | | 5.53 (dd, 1H), 6.69 (d, 1H), | |
| | phenyl]piperazin-1- | | | 6.87 (bs, 1H), 7.25 (d, 1H), | |
| | yl]ethanol | | | 7.50 (ddd, 1H), 7.75 (d, | |
| | | | | 1H), 8.45 (s, 1H), 8.72 (s, | |
| | | | | 1H), 8.78 (s, 1H), 9.53 (bs, | |
| | | | | 1H) | |
| 3.46^{a} | 5-chloro-4- | 2-methoxy-4-[4-(2- | 494 | 2.54 (t, 2H), 2.57-2.62 (m, | 45 |
| | imidazo[1,2-a]pyridin- | methoxyethyl)piperazin- | | 4H), 3.15-3.21 (m, 4H), | |
| | 3-yl-N-[2- | 1- | | 3.26 (s, 3H), 3.49 (t, 2H), | |
| | methoxy-4-[4-(2- | yl]aniline (Method | | 3.75 (s, 3H), 6.53 (dd, 1H), | |
| | methoxyethyl)piperazin- | 7) | | 6.69 (s, 1H), 6.88 (bs, 1H), | |
| | 1- | | | 7.26 (d, 1H), 7.50 (ddd, | |
| | yl]phenyl]pyrimidin- | | | 1H), 7.75 (d, 1H), 8.45 (s, | |
| | 2-amine | | | 1H), 8.72 (s, 1H), 8.79 (s, | |
| | | | | 1H), 9.53 (bs, 1H) | |
| 3.47^{a} | (3S)-1-[4-[(5- | (3S)-1-(4-amino-3- | 437 | 1.89-1.97 (m, 1H), | 32 |
| | chloro-4- | methoxyphenyl)pyrrolidin- | | 2.04-2.14 (m, 1H), | |
| | imidazo[1,2-a]pyridin- | 3-ol | | 3.12-3.17 (m, 1H), 3.33-3.36 (m | |
| | 3-yl- | (Method 7) | | partially hidden by H2O, | |
| | pyrimidin-2-yl)amino]- | | | 1H), 3.36-3.42 (m, 1H), | |
| | 3-methoxy- | | | 3.48 (dd, 1H), 3.74 (s, 3H), | |
| | phenyl]pyrrolidin- | | | 4.44 (bs, 1H), 5.00 (bs, | |
| | 3-ol | | | 1H), 6.13 (dd, 1H), | |
| | | | | 6.25 (d, 1H), 6.81 (bs, 1H), | |
| | | | | 7.14 (d, 1H), 7.45 (dd, 1H), | |
| | | | | 7.73 (d, 1H), 8.42 (s, 1H), | |
| | | | | 8.71 (s, 1H), 8.72 (s, 1H), | |
| | | | | 9.45 (bs, 1H) | |
| 3.48^{a} | 5-chloro-4- | 2-methoxy-4-(1,4- | 451 | 1.91-1.98 (m, 2H), | 21 |
| | imidazo[1,2-a]pyridin- | oxazepan-4- | | 3.67 (m, 6H), 3.74 (s, 3H), | |
| | 3-yl-N-[2- | yl)aniline (Method | | 3.75-3.79 (m, 2H), | |
| | methoxy-4-(1,4- | 7) | | 6.35 (dd, 1H), 6.45 (d, 1H), | |
| | oxazepan-4-yl)phenyl]pyrimidin- | | | 6.88 (bs, 1H), 7.15 (d, 1H), | |
| | 2-amine | | | 7.47 (dd, 1H), 7.74 (d, | |
| | | | | 1H), 8.43 (s, 1H), 8.71 (bs, | |
| | | | | 2H), 9.52 (bs, 1H) | |
| 3.49 | (3R)-1-[4-[(5- | (3R)-1-(4-amino-3- | 437 | 1.90-1.98 (m, 1H), | — |
| | chloro-4- | methoxyphenyl)pyrrolidin- | | 2.14 (m, 1H), | |
| | imidazo[1,2-a]pyridin- | 3-ol | | 3.12-3.17 (m, 1H), 3.33-3.36 (m | |
| | 3-yl- | (Method 7) | | partially hidden by H2O, | |
| | pyrimidin-2-yl)amino]- | | | 1H), 3.36-3.43 (m, 1H), | |
| | 3-methoxy- | | | 3.48 (dd, 1H), 3.74 (s, 3H), | |
| | phenyl]pyrrolidin- | | | 4.41-4.47 (m, 1H), 5.00 (d, | |
| | 3-ol | | | 1H), 6.13 (dd, 1H), | |
| | | | | 6.25 (d, 1H), 6.82 (bs, 1H), | |
| | | | | 7.14 (d, 1H), 7.45 (dd, 1H), | |
| | | | | 7.74 (d, 1H), 8.42 (s, 1H), | |
| | | | | 8.71 (s, 1H), 8.72 (s, 1H), | |
| | | | | 9.45 (bs, 1H) | |

Notes:
^{a}4-Methyl-2-pentanol was used as a solvent
^{b}The product was purified by flash chromatography on silica gel
^{c}5-chloro-4-imidazo[1,2-a]pyridin-3-yl-N-(2-methoxy-5-piperazin-1-yl-phenyl)pyrimidin-2-amine (Ex 3.10) was obtained in the same reaction as 1-[4-[3-[(5-chloro-4-imidazo[1,2-a]pyridin-3-yl-pyrimidin-2-yl)amino]-4-methoxy-phenyl]piperazin-1-yl]ethanone (Example 3.9). The two products were separated by flash chromatography on silica gel eluting with 0 to 8% methanolic ammonia (7N)/EtOAc.
^{d}The tert-butoxycarbonyl protecting group is cleaved during the reaction.

### Example 4

### 5-chloro-4-imidazo[1,2-a]pyridin-3-yl-N-[2-methoxy-4-(4-piperidyl)phenyl]pyrimidin-2-amine

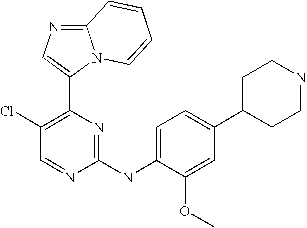

3-(2,5-dichloropyrimidin-4-yl)imidazo[1,2-a]pyridine (See Method 1, 1.90 g, 6.81 mmol), 1-(4-(4-amino-3-methoxyphenyl)piperidin-1-yl)ethanone (See Method 12, 1.69 g, 6.81 mmol) and p-toluenesulfonic acid (2.59 g, 13.6 mmol) were suspended in 2-pentanol (15 mL). The reaction mixture was placed in a sealed microwave vessel and heated at 140° C. during 2 hours in a microwave reactor. The solvent was evaporated and the residue was triturated in a 5% aqueous solution of potassium carbonate. The mixture was extracted with dichloromethane containing 5% of methanol. The organic phase was dried over magnesium sulfate and concentrated to afford the crude product which was purified by flash chromatography on silica gel eluting with 0 to 10% methanol in dichloromethane followed by 10% NH3/MeOH (7N) in dichloromethane. 5-chloro-4-imidazo[1,2-a]pyridin-3-yl-N-[2-methoxy-4-(4-piperidyl)phenyl]pyrimidin-2-amine (1.53 g, 51%) was obtained as a pale yellow solid; NMR spectrum: 1.52-1.63 (m, 2H), 1.69-1.77 (m, 2H), 2.51-2.53 (m partially hidden by DMSOd5, 1H), 2.56-2.64 (m, 2H), 3.01-3.08 (m, 2H), 3.79 (s, 3H), 6.84 (dd, 1H), 6.91 (dd, 1H), 6.99 (d, 1H), 7.46 (d, 1H), 7.51 (dd, 1H), 7.76 (d, 1H), 8.50 (s, 1H), 8.73 (s, 1H), 8.88 (s, 1H), 9.60 (bs, 1H); Mass spectrum: 435 (MH+).

### Example 5

### 5-chloro-4-imidazo[1,2-a]pyridin-3-yl-N-[2-methoxy-4-(4-piperidyloxy)phenyl]pyrimidin-2-amine

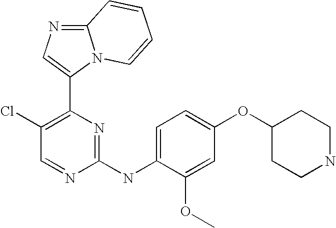

3-(2,5-dichloropyrimidin-4-yl)imidazo[1,2-a]pyridine (see Method 1, 200 mg, 0.75 mmol), tert-butyl 4-(4-amino-3-methoxyphenoxy)piperidine-1-carboxylate (see Method 26, 316 mg, 0.98 mmol) and p-toluenesulfonic acid (287 mg, 1.51 mmol) were suspended in 2-pentanol (3 mL) and DMA (0.8 mL) and heated at 130° C. for 8 hours. The solution was concentrated to dryness, the residue was dissolved in the minimum of DMA and 3 drops of a 30% aqueous ammonia was added. The solution was purified by preparative HPLC using a Waters X-Terra reverse-phase column (5 microns silica, 30 mm diameter, 150 mm length) and decreasingly polar mixtures of water (containing 0.2% ammonium carbonate) and acetonitrile as eluent. The fractions were evaporated to afford 5-chloro-4-(imidazo[1,2-a]pyridin-3-yl)-N-(2-methoxy-4-(piperidin-4-yloxy)phenyl)pyrimidin-2-amine (218 mg, 64%); NMR spectrum: 1.42-1.53 (m, 2H), 1.89-1.99 (m, 2H), 2.55-2.63 (m, 2H), 2.91-3.02 (m, 2H), 3.22 (bs, 1H), 3.75 (s, 3H), 4.39-4.50 (m, 1H), 6.59 (d, 1H), 6.71 (s, 1H), 6.88 (bs, 1H), 7.31 (d, 1H), 7.49 (dd, 1H), 7.75 (d, 1H), 8.47 (s, 1H), 8.72 (s, 1H), 8.85 (s, 1H), 9.52 (bs, 1H); Mass spectrum: 451 (MH+).

### Example 6

The following compounds were prepared using a similar procedure as described in Example 5 using 3-(2,5-dichloropyrimidin-4-yl)imidazo[1,2-a]pyridine and the appropriate aniline. The anilines used as the starting material are indicated in the following table and were prepared as described in the Methods section below.

| Ex. No. | Name | Starting aniline | MH+ | NMR spectrum | % Yield |
|---|---|---|---|---|---|
| 6.1 | 5-chloro-4- | tert-butyl(2R)-2- | 451 | 1.43-1.52 (m, 1H), | 79 |
| | imidazo[1,2- | [(4-amino-3- | | 1.60-1.77 (m, 2H), | |
| | a]pyridin-3-yl-N- | methoxyphenoxy)methyl]pyrrolidine- | | 1.82-1.92 (m, 1H), 1.97 (bs, 1H), | |
| | [2-methoxy-4- | 1- | | 2.77-2.89 (m, 2H), | |
| | [[(2R)-pyrrolidin- | carboxylate | | 3.37-3.44 (m, 1H), 3.76 (s, 3H), | |
| | 2- | (Method 27) | | 3.82-3.90 (m, 2H), | |
| | yl]methoxy]phenyl]pyrimidin- | | | 6.56 (dd, 1H), 6.71 (d, 1H), | |
| | 2- | | | 6.90 (bs, 1H), 7.33 (d, 1H), | |
| | amine | | | 7.48 (ddd, 1H), 7.75 (d, | |
| | | | | 1H), 8.46 (s, 1H), 8.72 (s, | |
| | | | | 1H), 8.85 (bs, 1H), | |
| | | | | 9.52 (bs, 1H) | |
| 6.2 | 5-chloro-4- | tert-butyl(2S)-2- | 451 | 1.43-1.53 (m, 1H), | 45 |
| | imidazo[1,2- | [(4-amino-3- | | 1.60-1.77 (m, 2H), | |
| | a]pyridin-3-yl-N- | methoxyphenoxy)methyl]pyrrolidine- | | 1.82-1.92 (m, 1H), 1.96 (bs, 1H), | |
| | [2-methoxy-4- | 1- | | 2.77-2.89 (m, 2H), | |
| | [[(2S)-pyrrolidin- | carboxylate | | 3.37-3.45 (m, 1H), 3.76 (s, 3H), | |
| | 2- | (Method 27) | | 3.82-3.90 (m, 2H), | |
| | yl]methoxy]phenyl]pyrimidin- | | | 6.56 (dd, 1H), 6.71 (d, 1H), | |
| | 2- | | | 6.90 (bs, 1H), 7.33 (d, 1H), | |
| | amine | | | 7.48 (ddd, 1H), 7.76 (d, | |
| | | | | 1H), 8.46 (s, 1H), 8.72 (s, | |
| | | | | 1H), 8.84 (bs, 1H), | |
| | | | | 9.52 (bs, 1H) | |
| 6.3 | 5-chloro-4- | tert-butyl(2S)-2- | 451 | 1.32-1.42 (m, 1H), | 32 |
| | imidazo[1,2-a]pyridin- | [(3-amino-4- | | 1.54-1.70 (m, 2H), | |
| | 3-yl-N-[2- | methoxyphenoxy)methyl]pyrrolidine- | | 1.72-1.83 (m, 1H), 2.72-2.82 (m, | |
| | methoxy-5-[[(2S)- | 1- | | 2H), 3.28-3.33 (m partially | |
| | pyrrolidin-2-yl]methoxy]phenyl]pyrimidin- | carboxylate | | hidden by H2O, 1H), | |
| | 2-amine | (Method 27) | | 3.66-3.75 (m, 2H), 3.77 (s, 3H), | |
| | | | | 6.73 (dd, 1H), 7.00 (dd, | |
| | | | | 1H), 7.02 (d, 1H), 7.38 (d, | |
| | | | | 1H), 7.51 (ddd, 1H), | |
| | | | | 7.78 (d, 1H), 8.57 (s, 1H), | |
| | | | | 8.72 (s, 1H), 8.82 (s, 1H), | |
| | | | | 9.64 (bs, 1H) | |
| 6.4 | 5-chloro-4- | tert-butyl(2R)-2- | 451 | 1.33-1.43 (m, 1H), | 40 |
| | imidazo[1,2-a]pyridin- | [(3-amino-4- | | 1.55-1.71 (m, 2H), | |
| | 3-yl-N-[2- | methoxyphenoxy)methyl]pyrrolidine- | | 1.73-1.83 (m, 1H), 2.72-2.82 (m, | |
| | methoxy-5- | 1- | | 2H), 3.28-3.33 (m partially | |
| | [[(2R)-pyrrolidin- | carboxylate | | hidden by H2O, 1H), | |
| | 2-yl]methoxy]phenyl]pyrimidin- | (Method 27) | | 3.66-3.75 (m, 2H), 3.77 (s, 3H), | |
| | 2- | | | 6.73 (dd, 1H), 7.00 (dd, | |
| | amine | | | 1H), 7.02 (d, 1H), 7.39 (d, | |
| | | | | 1H), 7.51 (ddd, 1H), | |
| | | | | 7.78 (d, 1H), 8.57 (s, 1H), | |
| | | | | 8.72 (s, 1H), 8.82 (s, 1H), | |
| | | | | 9.64 (bs, 1H) | |
| 6.5 | 5-chloro-4- | tert-butyl(3R)-3- | 437 | 1.76-1.84 (m, 1H), | 63 |
| | imidazo[1,2-a]pyridin- | (4-amino-3- | | 1.99-2.09 (m, 1H), | |
| | 3-yl-N-[2- | methoxyphenoxy)pyrrolidine- | | 2.76-2.84 (m, 1H), 2.88 (dd, 1H), | |
| | methoxy-4-[(3R)- | 1-carboxylate | | 2.91-2.97 (m, 1H), | |
| | pyrrolidin-3- | (Method 26) | | 3.09 (dd, 1H), 3.75 (s, 3H), | |
| | yl]oxy- | | | 4.88-4.94 (m, 1H), | |
| | phenyl]pyrimidin- | | | 6.54 (dd, 1H), 6.67 (d, 1H), | |
| | 2-amine | | | 6.89 (bs, 1H), 7.33 (d, 1H), | |
| | | | | 7.49 (ddd, 1H), 7.76 (d, | |
| | | | | 1H), 8.46 (s, 1H), 8.72 (s, | |
| | | | | 1H), 8.84 (s, 1H), 9.53 (bs, | |
| | | | | 1H) | |
| 6.6 | 5-chloro-4- | tert-butyl(3S)-3-(4- | 437 | 1.74-1.83 (m, 1H), | 56 |
| | imidazo[1,2-a]pyridin- | amino-3-methoxyphenoxy)pyrrolidine- | | 1.99-2.08 (m, 1H), | |
| | 3-yl-N-[2- | 1-carboxylate | | 2.75-2.82 (m, 1H), 2.87 (dd, 1H), | |
| | methoxy-4-[(3S)- | (Method 26) | | 2.90-2.97 (m, 1H), | |
| | pyrrolidin-3- | | | 3.08 (dd, 1H), 3.75 (s, 3H), | |
| | yl]oxy- | | | 4.86-4.93 (m, 1H), | |
| | phenyl]pyrimidin- | | | 6.54 (dd, 1H), 6.67 (d, 1H), | |
| | 2-amine | | | 6.89 (bs, 1H), 7.33 (d, 1H), | |
| | | | | 7.49 (ddd, 1H), 7.76 (d, | |
| | | | | 1H), 8.47 (s, 1H), 8.72 (s, | |
| | | | | 1H), 8.84 (s, 1H), 9.53 (bs, | |
| | | | | 1H) | |

### Example 7

### 1-[4-[4-[(5-chloro-4-imidazo[1,2-a]pyridin-3-yl-pyrimidin-2-yl)amino]-3-methoxy-phenoxy]-1-piperidyl]ethanone

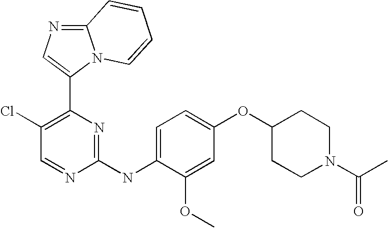

A mixture of 5-chloro-4-(imidazo[1,2-a]pyridin-3-yl)-N-(2-methoxy-4-(piperidin-4-yloxy)phenyl)pyrimidin-2-amine (Example 5, 100 mg, 0.22 mmol) and acetic anhydride (0.37 ml, 3.33 mmol) in dichloromethane (4 ml) was stirred at room temperature for 3 hours. After concentration, 7N methanolic ammonia was added till pH=9, the solution was concentrated and the residue was purified by preparative HPLC using a Waters X-Terra reverse-phase column (5 microns silica, 30 mm diameter, 150 mm length) and decreasingly polar mixtures of water (containing 0.2% ammonium carbonate) and acetonitrile as eluent. The fractions were evaporated to dryness to afford 1-(4-(4-(5-chloro-4-(imidazo[1,2-a]pyridin-3-yl)pyrimidin-2-ylamino)-3-methoxyphenoxy)piperidin-1-yl)ethanone (75 mg, 68%); NMR spectrum: 1.49-1.59 (m, 1H), 1.60-1.70 (m, 1H), 1.88-1.96 (m, 1H), 1.96-2.05 (m, 1H), 2.04 (s, 3H), 3.23-3.30 (m, 1H), 3.40-3.40 (m partially hidden by H2O, 1H), 3.65-3.74 (m, 1H), 3.76 (s, 3H), 3.83-3.92 (m, 1H), 4.63-4.70 (m, 1H), 6.64 (dd, 1H), 6.76 (d, 1H), 6.89 (bs, 1H), 7.35 (d, 1H), 7.50 (ddd, 1H), 7.76 (d, 1H), 8.47 (s, 1H), 8.72 (s, 1H), 8.86 (s, 1H), 9.53 (bs, 1H); Mass spectrum: 493 (MH+).

### Example 8

### 5-chloro-4-imidazo[1,2-a]pyridin-3-yl-N-[2-methoxy-4-[(1-methylsulfonyl-4-piperidyl)oxy]phenyl]pyrimidin-2-amine

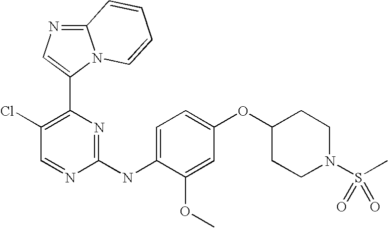

Methanesulfonyl chloride (0.022 ml, 0.29 mmol) was added to a stirred solution of 5-chloro-4-(imidazo[1,2-a]pyridin-3-yl)-N-(2-methoxy-4-(piperidin-4-yloxy)phenyl)pyrimidin-2-amine (Example 5, 108 mg, 0.24 mmol) and triethylamine (0.050 ml, 0.36 mmol) in dichloromethane (3 ml) at 0° C. under argon. The resulting solution was stirred at 25° C. for 3 hours, then concentrated and purified by preparative HPLC using a Waters X-Terra reverse-phase column (5 microns silica, 30 mm diameter, 150 mm length) and decreasingly polar mixtures of water (containing 0.2% ammonium carbonate) and acetonitrile as eluent. The fractions were evaporated to dryness to afford 5-chloro-4-(imidazo[1,2-a]pyridin-3-yl)-N-(2-methoxy-4-(1-(methylsulfonyl)piperidin-4-yloxy)phenyl)pyrimidin-2-amine (85 mg, 67%);

NMR spectrum: 1.72-1.84 (m, 2H), 1.99-2.10 (m, 2H), 2.93 (s, 3H), 3.10-3.20 (m, 2H), 3.46-3.45 (m, 2H), 3.77 (s, 3H), 3.58-3.66 (m, 1H), 6.64 (dd, 1H), 6.77 (d, 1H), 6.89 (bs, 1H), 7.36 (d, 1H), 7.50 (ddd, 1H), 7.76 (d, 1H), 8.47 (s, 1H), 8.72 (s, 1H), 8.86 (s, 1H), 9.54 (bs, 1H);

Mass spectrum: 529 (MH+).

### Example 9

### 1-[4-[4-[(5-chloro-4-imidazo[1,2-a]pyridin-3-yl-pyrimidin-2-yl)amino]-3-methoxy-phenoxy]-1-piperidyl]-2-hydroxy-propan-1-one

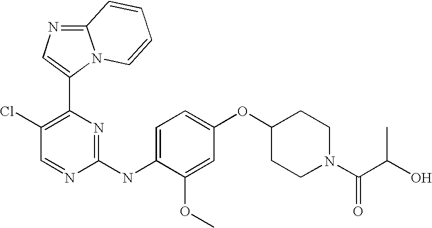

2-(1H-benzo[d][1,2,3]triazol-1-yl)-1,1,3,3-tetramethylisouronium tetrafluoroborate (94 mg, 0.29 mmol) was added to a stirred solution of 5-chloro-4-(imidazo[1,2-a]pyridin-3-yl)-N-(2-methoxy-4-(piperidin-4-yloxy)phenyl)pyrimidin-2-amine (Example 5, 106 mg, 0.24 mmol), N,N-diisopropylethylamine (0.082 ml, 0.47 mmol) and 2-hydroxypropanoic acid (0.026 ml, 0.35 mmol) in N-methyl-2-pyrrolidinone (1 ml) at 0° C. under argon. The resulting solution was stirred at room temperature for 2 hours. The reaction mixture was quenched with a saturated solution of sodium bicarbonate and extracted with ethyl acetate. The combined organic layers were washed with water, brine, dried over magnesium sulfate then purified by flash chromatography on silica gel eluting with 3% methanol in dichloromethane. The solvent was evaporated to dryness, the gum was triturated in diethyl ether to afford 1-(4-(4-(5-chloro-4-(imidazo[1,2-a]pyridin-3-yl)pyrimidin-2-ylamino)-3-methoxyphenoxy)piperidin-1-yl)-2-hydroxypropan-1-one (83 mg, 67%); NMR spectrum: 1.20 (d, 3H), 1.50-1.71 (m, 2H), 1.91-2.05 (m, 2H), 3.21-3.29 (m, 0.5H, 3.34-3.48 (m, 1.5H), 3.76 (s, 3H), 3.78-3.88 (m, 1.5H), 3.91-3.98 (m, 0.5H), 4.44-4.50 (m, 1H), 4.69 (bs, 1H), 4.86-4.92 (m, 1H), 6.64 (dd, 1H), 6.76 (d, 1H), 6.90 (bs, 1H) 7.35 (d, 1H), 7.50 (ddd, 1H), 7.76 (d, 1H), 8.47 (s, 1H), 8.71 (s, 1H), 8.86 (s, 1H), 9.54 (bs, 1H); Mass spectrum: 523 (MH+).

### Example 9A

### (2S)-1-[4-[4-[(5-chloro-4-imidazo[1,2-a]pyridin-3-yl-pyrimidin-2-yl)amino]-3-methoxy-phenoxy]-1-piperidyl]-2-hydroxy-propan-1-one

Enantiomerically pure (2S)-1-[4-[4-[(5-chloro-4-imidazo[1,2-a]pyridin-3-yl-pyrimidin-2-yl)amino]-3-methoxy-phenoxy]-1-piperidyl]-2-hydroxy-propan-1-one (76%) was obtained following the same procedure as Example 9 but using (S)-2-hydroxypropanoic acid and 5-chloro-4-(imidazo[1,2-a]pyridin-3-yl)-N-(2-methoxy-4-(piperidin-4-yloxy)phenyl)pyrimidin-2-amine.

### Example 10

### 1-[(3R)-3-[4-[(5-chloro-4-imidazo[1,2-a]pyridin-3-yl-pyrimidin-2-yl)amino]-3-methoxy-phenoxy]pyrrolidin-1-yl]ethanone

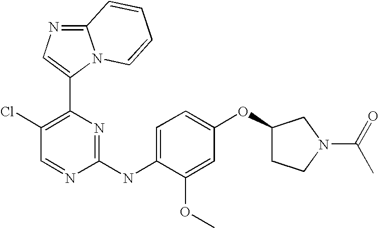

This compound was prepared following the procedure described in Example 7 using 5-chloro-4-imidazo[1,2-a]pyridin-3-yl-N-[2-methoxy-4-[(3R)-pyrrolidin-3-yl]oxy-phenyl]pyrimidin-2-amine (Example 6.5) and acetic anhydride as the starting materials (66%); NMR spectrum: 1.94 (s, 1.5H), 2.01 (s, 1.5H), 2.03-2.29 (m, 2H), 3.33-3.40 (m, 0.5H), 3.51-3.67 (m, 3H), 3.75 (s, 1.5H), 3.76 (s, 1.5H), 3.83 (dd, 0.5H), 5.08 (bs, 0.5H), 5.15 (bs, 0.5H), 6.60 (ddd, 1H), 6.74 (dd, 1H), 6.90 (bs, 1H), 7.37 (dd, 1H), 7.48 (ddd, 1H), 7.76 (d, 1H), 8.47 (s, 0.5H), 8.48 (s, 0.5H), 8.71 (s, 0.5H), 8.72 (s, 0.5H), 8.86 (s, 0.5H), 8.87 (s, 0.5H), 9.53 (bs, 1H); Mass spectrum: 479 (MH+).

### Example 11

### 5-chloro-4-imidazo[1,2-a]pyridin-3-yl-N-[2-methoxy-4-[(3R)-1-methylsulfonylpyrrolidin-3-yl]oxy-phenyl]pyrimidin-2-amine

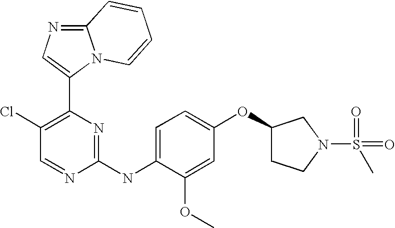

This compound was prepared following the procedure described in Example 8 using 5-chloro-4-imidazo[1,2-a]pyridin-3-yl-N-[2-methoxy-4-[(3R)-pyrrolidin-3-yl]oxy-phenyl]pyrimidin-2-amine (Example 6.5) and methanesulfonyl chloride as the starting materials (66%); NMR spectrum: 2.10-2.17 (m, 1H), 2.20-2.30 (m, 1H), 2.93 (s, 3H), 3.39-3.47 (m, 3H), 3.62 (dd, 1H), 3.76 (s, 3H), 5.12 (bs, 1H), 6.60 (dd, 1H), 6.74 (d, 1H), 6.91 (bs, 1H), 7.38 (d, 1H), 7.49 (ddd, 1H), 7.76 (d, 1H), 8.48 (s, 1H), 8.72 (s, 1H), 8.87 (s, 1H), 9.54 (bs, 1H); Mass spectrum: 515 (MH+).

### Example 12

### 1-[(3R)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one

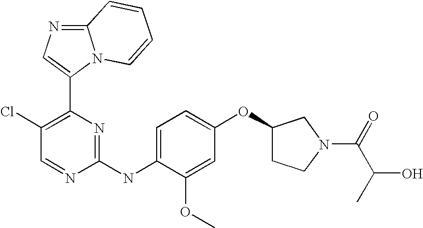

This compound was obtained as a mixture of diastereoisomers following the procedure described in Example 9 using 5-chloro-4-imidazo[1,2-a]pyridin-3-yl-N-[2-methoxy-4-[(3R)-pyrrolidin-3-yl]oxy-phenyl]pyrimidin-2-amine (Example 6.5) and 2-hydroxypropanoic acid as the starting materials (74%); NMR spectrum: 1.14 (d, 1.5H), 1.18 (d, 1.5H), 2.04-2.15 (m, 1H), 2.16-2.28 (m, 1H), 3.39-3.95 (m, 4H), 3.75 (s, 1.5H), 3.76 (s, 1.5H), 4.19-4.28 (m, 0.5H), 4.29-4.37 (m, 0.5H), 4.93-4.98 (m, 1H), 5.09 (bs, 0.5H), 5.16 (bs, 0.5H), 6.57-6.64 (m, 1H), 6.71-6.76 (m, 1H), 6.90 (bs, 1H), 7.37 (dd, 1H), 7.45-7.53 (m, 1H), 7.76 (d, 1H), 7.47 (s, 0.5H), 7.48 (s, 0.5H), 8.71 (s, 0.5H), 8.72 (s, 0.5H), 8.74 (s, 1H), 9.54 (bs, 1H); Mass spectrum: 509 (MH+).

### Example 12A

### (2S)-1-[(3R)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one

Following the same procedure as Example 12 but using (S)-2-hydroxypropanoic acid, (2S)-1-[(3R)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one was obtained as single diastereoisomer (34%); NMR spectrum: 1.18 (d, 1.5H), 1.22 (d, 1.5H), 2.05-2.14 (m, 1H), 2.16-2.24 (m, 1H), 3.41-3.49 (m, 0.5H), 3.52-3.62 (m, 1.5H), 3.65 (dd, 0.5H), 3.75 (s, 1.5H), 3.76 (s, 1.5H), 3.79 (dd, 0.5H), 3.82-3.89 (m, 1H), 4.21-4.28 (m, 0.5H), 4.31-4.37 (m, 0.5H), 4.94 (d, 0.5H), 4.95 (d, 0.5H), 5.09 (bs, 0.5H), 5.16 (bs, 0.5H), 6.57-6.63 (m, 1H), 6.71-6.75 (m, 1H), 6.90 (bs, 1H), 7.37 (dd, 1H), 7.45-7.52 (m, 1H), 7.76 (d, 1H), 8.47 (s, 0.5H), 8.48 (s, 0.5H), 8.71 (s, 0.5H), 8.72 (s, 0.5H), 8.87 (s, 1H), 9.55 (bs, 1H).

### Example 13

### 1-[(3S)-3-[4-[(5-chloro-4-imidazo[1,2-a]pyridin-3-yl-pyrimidin-2-yl)amino]-3-methoxy-phenoxy]pyrrolidin-1-yl]ethanone

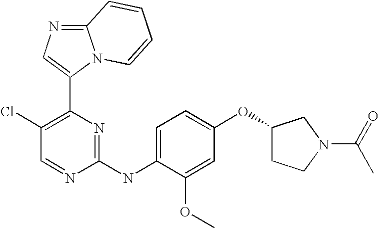

This compound was prepared following the procedure described in Example 7 using 5-chloro-4-imidazo[1,2-a]pyridin-3-yl-N-[2-methoxy-4-[(3S)-pyrrolidin-3-yl]oxy-phenyl]pyrimidin-2-amine (Example 6.6) and acetic anhydride as the starting materials (84%); NMR spectrum: 1.94 (s, 1.5H), 2.00 (s, 1.5H), 2.04-2.29 (m, 2H), 3.34-3.41 (m, 0.5H), 3.52-3.68 (m, 3H), 3.75 (s, 1.5H), 3.77 (s, 1.5H), 3.83 (dd, 0.5H), 5.06-5.11 (m, 0.5H), 5.13-5.18 (m, 0.5H), 6.59 (dd, 0.5H), 6.61 (dd, 0.5H), 6.72 (d, 0.5H), 6.74 (d, 0.5H), 6.90 (bs, 0.5H), 6.91 (bs, 0.5H), 7.33-7.41 (m, 1H), 7.45-7.52 (m, 1H), 7.72-7.79 (m, 1H), 8.47 (s, 0.5H), 8.48 (s, 0.5H), 8.71 (s, 0.5H), 8.72 (s, 0.5H), 8.86 (s, 0.5H), 8.87 (s, 0.5H), 9.53 (bs, 1H); Mass spectrum: 479 (MH+).

### Example 14

### 5-chloro-4-imidazo[1,2-a]pyridin-3-yl-N-[2-methoxy-4-[(3S)-1-methylsulfonylpyrrolidin-3-yl]oxy-phenyl]pyrimidin-2-amine

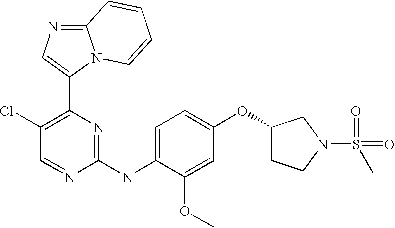

This compound was prepared following the procedure described in Example 8 using 5-chloro-4-imidazo[1,2-a]pyridin-3-yl-N-[2-methoxy-4-[(3S)-pyrrolidin-3-yl]oxy-phenyl]pyrimidin-2-amine (Example 6.6) and methanesulfonyl chloride as the starting materials (91%); NMR spectrum: 2.12-2.20 (m, 1H), 2.23-2.33 (m, 1H), 2.96 (s, 3H), 3.41-3.50 (m, 3H), 3.64 (dd, 1H), 3.79 (s, 3H), 5.13-5.18 (m, 1H), 6.63 (dd, 1H), 6.76 (d, 1H), 6.93 (bs, 1H), 7.40 (d, 1H), 7.51 (ddd, 1H), 7.79 (d, 1H), 8.50 (s, 1H), 8.74 (s, 1H), 8.90 (s, 1H), 9.57 (bs, 1H); Mass spectrum: 515 (MH+).

### Example 15

### 1-[(3S)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one

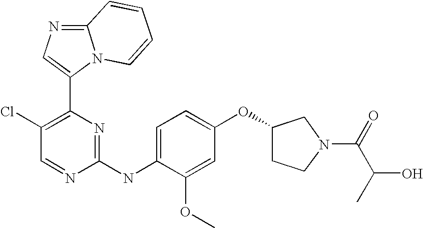

This compound was obtained as a mixture of diastereoisomers following the procedure described in Example 9 using 5-chloro-4-imidazo[1,2-a]pyridin-3-yl-N-[2-methoxy-4-[(3S)-pyrrolidin-3-yl]oxy-phenyl]pyrimidin-2-amine (Example 6.6) and 2-hydroxypropanoic acid as the starting materials (66%); NMR spectrum: 1.14 (d, 0.75H), 1.18 (d, 0.75H), 1.22 (d, 1.5H), 2.04-2.29 (m, 2H), 3.40-3.49 (m, 0.5H), 3.52-3.72 (m, 3H), 3.75 (s, 1.5H), 3.76 (s, 1.5H), 3.81-3.88 (m, 0.5H), 4.18-4.28 (m, 0.5H), 4.29-4.38 (m, 0.5H), 4.91-4.99 (m, 1H), 5.09 (bs, 0.5H), 5.16 (bs, 0.5H), 6.60 (dd, 0.5H), 6.61 (dd, 0.5H), 6.73 (d, 0.5H), 7.74 (d, 0.5H), 6.90 (bs, 1H), 7.33-7.41 (m, 1H), 7.45-7.52 (m, 1H), 7.76 (d, 1H), 8.47 (s, 0.5H), 8.48 (s, 0.5H), 8.72 (s, 0.5H), 8.73 (s, 0.5H), 8.87 (s, 1H), 9.54 (bs, 1H); Mass spectrum: 509 (MH+).

### Example 15A

### (2S)-1-[(3S)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one

Following the same procedure as Example 15 but using (S)-2-hydroxypropanoic acid, (2S)-1-[(3S)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one was obtained as single diastereoisomer (72%); NMR spectrum: 1.14 (d, 1.5H), 1.22 (d, 1.5H), 2.04-2.29 (m, 2H), 3.40-3.47 (m, 0.5H), 3.56-3.72 (m, 3H), 3.75 (s, 1.5H), 3.77 (s, 1.5H), 3.92 (dd, 0.5H), 4.18-4.25 (m, 0.5H), 4.29-4.37 (m, 0.5H), 4.95 (d, 0.5H), 4.96 (d, 0.5H), 5.07-5.11 (m, 0.5H), 5.14-5.19 (m, 0.5H), 6.60 (dd, 0.5H), 6.62 (dd, 0.5H), 6.73 (d, 0.5H), 6.74 (d, 0.5H), 6.90 (bs, 1H), 7.34-7.41 (m, 1H), 7.46-7.53 (m, 1H), 7.72-7.79 (m, 1H), 8.47 (s, 0.5H), 8.48 (s, 0.5H), 8.71 (s, 0.5H), 8.72 (s, 0.5H), 8.87 (s, 1H), 9.53 (bs, 1H).

### Example 16

### 1-[(2S)-2-[[4-[(5-chloro-4-imidazo[1,2-a]pyridin-3-yl-pyrimidin-2-yl)amino]-3-methoxy-phenoxy]methyl]pyrrolidin-1-yl]ethanone

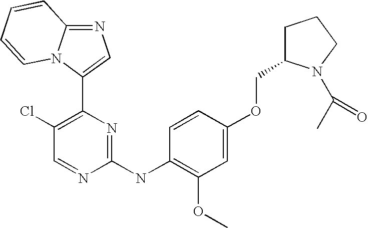

This compound was prepared following the procedure described in Example 7 using 5-chloro-4-imidazo[1,2-a]pyridin-3-yl-N-[2-methoxy-4-[[(2S)-pyrrolidin-2-yl]methoxy]phenyl]pyrimidin-2-amine (Example 6.2) and acetic anhydride as the starting materials (70%); NMR spectrum: 1.93-2.00 (m, 3H), 1.99 (s, 3H), 2.02-2.11 (m, 1H), 3.40-3.48 (m, 1H), 3.49-3.55 (m, 1H), 3.77 (s, 3H), 3.87-3.93 (m, 1H), 4.16 (dd, 1H), 3.23-3.30 (m, 1H), 6.60 (dd, 1H), 6.85 (d, 1H), 6.88 (bs, 1H), 7.33 (d, 1H), 7.48 (ddd, 1H), 7.75 (d, 1H), 8.46 (s, 1H), 8.72 (s, 1H), 8.85 (s, 1H), 9.52 (bs, 1H); Mass spectrum: 493 (MH+).

### Example 17

### 1-[(2R)-2-[[3-[(5-chloro-4-imidazo[1,2-a]pyridin-3-yl-pyrimidin-2-yl)amino]-4-methoxy-phenoxy]methyl]pyrrolidin-1-yl]ethanone

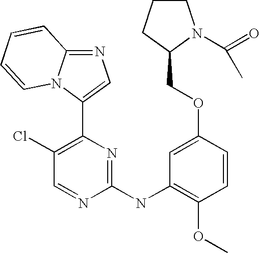

This compound was prepared following the procedure described in Example 7 using 5-chloro-4-imidazo[1,2-a]pyridin-3-yl-N-[2-methoxy-5-[[(2R)-pyrrolidin-2-yl]methoxy]phenyl]pyrimidin-2-amine (Example 6.4) and acetic anhydride as the starting materials (77%); NMR spectrum: 1.81-1.91 (m, 3H), 1.93 (s, 3H), 1.93-2.00 (m, 1H), 3.34-3.46 (m, 2H), 3.76 (s, 3H), 3.78-3.92 (m, 1H), 4.01 (dd, 1H), 4.14-4.20 (m, 1H), 6.79 (dd, 1H), 6.97 (dd, 1H), 7.03 (d, 1H), 7.38 (d, 1H), 7.51 (ddd, 1H), 7.78 (d, 1H), 8.57 (s, 1H), 8.71 (s, 1H), 8.83 (s, 1H), 9.62 (bs, 1H); Mass spectrum: 493 (MH+).

### Example 18

### 4-[4-[(5-chloro-4-imidazo[1,2-a]pyridin-3-yl-pyrimidin-2-yl)amino]-3-methoxy-phenyl]piperazin-2-one

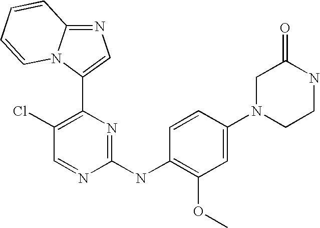

A stirred mixture of 3-(2,5-dichloropyrimidin-4-yl)imidazo[1,2-a]pyridine (Method 1, 180 mg, 0.68 mmol), 4-(4-amino-3-methoxyphenyl)piperazin-2-one (Method 7, 150 mg, 0.68 mmol), 9,9-dimethyl-4,5-bis(diphenylphosphino)xanthene (31.4 mg, 0.05 mmol), bis(dibenzylideneacetone)palladium (15.6 mg, 0.03 mmol) and cesium carbonate (442 mg, 1.36 mmol) in 1,4-dioxane (1.5 ml) was heated at 130° C. for 3 hours. After cooling, the solvent was evaporated and residue was purified by preparative HPLC using a Waters X-Terra reverse-phase column (5 microns silica, 30 mm diameter, 150 mm length) and decreasingly polar mixtures of water (containing 0.2% ammonium carbonate) and acetonitrile as eluent. The fractions were evaporated to dryness to afford 4-(4-(5-chloro-4-(imidazo[1,2-a]pyridin-3-yl)pyrimidin-2-ylamino)-3-methoxyphenyl)piperazin-2-one (35 mg, 11%); NMR spectrum: 3.35-3.40 (m partially hidden by H2O, 2H, 3.44-3.50 (m, 2H), 3.78 (s, 3H), 3.79 (s, 2H), 6.53 (dd, 1H), 6.70 (d, 1H), 6.90 (bs, 1H), 7.29 (d, 1H), 7.49 (ddd, 1H), 7.75 (d, 1H), 8.09 (bs, 1H), 8.45 (s, 1H), 8.72 (s, 1H), 8.80 (s, 1H), 9.54 (bs, 1H); Mass spectrum: 450 (MH+).

### Example 19

### 4-[4-[(5-chloro-4-imidazo[1,2-a]pyridin-3-yl-pyrimidin-2-yl)amino]-3-methoxy-phenyl]-N,N-dimethyl-piperazine-2-carboxamide

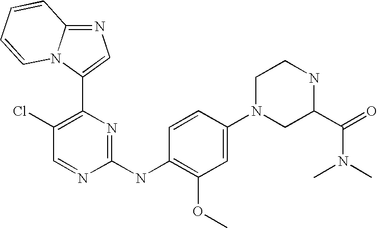

A mixture of 4-(4-amino-3-methoxyphenyl)-N,N-dimethylpiperazine-2-carboxamide (see Method 36; 205 mg, 0.38 mmol), 3-(2,5-dichloropyrimidin-4-yl)imidazo[1,2-a]pyridine (see Method 1, 100 mg, 0.38 mmol), 9,9-dimethyl-4,5-bis(diphenylphosphino)xanthene (21.8 mg, 0.04 mmol), tris(dibenzylideneacetone)dipalladium (8.6 mg, 0.009 mmol) and 1,8-diazabicyclo[5.4.0]-undec-7-ene (0.14 ml, 0.94 mmol) in dioxane (1.5 ml) was placed into a microwave tube and degazed with argon. The reaction vessel was sealed and heated at 140° C. for 15 minutes. The reaction mixture was purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent. The fractions containing the desired compound were evaporated to dryness to afford 4-(4-(5-chloro-4-(imidazo[1,2-a]pyridin-3-yl)pyrimidin-2-ylamino)-3-methoxyphenyl)-N,N-dimethylpiperazine-2-carboxamide (20 mg, 10%); NMR spectrum: 2.50-2.54 (m partially hidden by DMSOd5, 1H), 2.55-2.62 (m, 1H), 2.81-2.91 (m, 1H), 2.86 (s, 3H), 3.01-3.07 (m, 1H), 3.11 (s, 3H), 3.54-3.60 (m, 1H), 3.60-3.66 (m, 1H), 3.76 (s, 3H), 3.78-3.83 (m, 1H), 6.54 (dd, 1H), 6.69 (d, 1H), 6.89 (bs, 1H), 7.27 (d, 1H), 7.48 (ddd, 1H), 7.75 (d, 1H), 8.45 (s, 1H), 8.72 (s, 1H) 8.79 (s, 1H), 9.54 (bs, 1H); Mass spectrum: 507 (MH+).

### Example 20

### (3S)-1-[[4-[(5-chloro-4-imidazo[1,2-a]pyridin-3-yl-pyrimidin-2-yl)amino]-3-methoxy-phenyl]methyl]pyrrolidin-3-ol

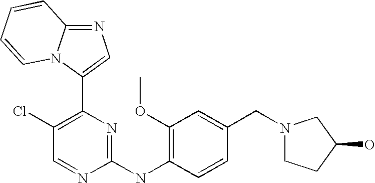

A solution of tetramethylammonium triacetoxyborohydrate (208 mg, 0.79 mmol) in DMA (300 μl) and THF (300 μl) was added to a mixture of (S)-pyrrolidin-3-ol (21 μl, 0.26 mmol), 4-(5-chloro-4-(imidazo[1,2-a]pyridin-3-yl)pyrimidin-2-ylamino)-3-methoxybenzaldehyde (Example 20A, 100 mg, 0.26 mmol), trimethyl orthoformate (200 μl, 1.8 mmol), methanol (50 μl) in DMA (750 μl). The solution was stirred at 20° C. overnight. After concentration, the reaction mixture was purified by preparative HPLC using a Waters

X-Terra reverse-phase column (C-18, 5 microns silica, 19 mm diameter, 50 mm length, flow rate of 40 ml/minute) and decreasingly polar mixtures of water (containing 0.2% ammonium carbonate) and acetonitrile as eluent. The fractions containing the desired compound were evaporated to dryness to give (3S)-1-(4-(5-chloro-4-(imidazo[1,2-a]pyridin-3-yl)pyrimidin-2-ylamino)-3-methoxybenzyl)pyrrolidin-3-ol (55 mg, 46%); NMR spectrum: 1.52-1.61 (m, 1H), 1.97-2.06 (m, 1H), 2.35 (dd, 1H), 2.40-2.47 (m, 1H), 2.56-2.64 (m, 1H), 2.70 (dd, 1H), 3.56 (d, 1H), 3.61 (d, 1H), 3.78 (s, 3H), 4.18-4.25 (m, 1H), 4.70 (d, 1H), 6.91 (dd, 1H), 6.93 (dd, 1H), 7.06 (d, 1H), 7.45-7.54 (m, 2H), 7.76 (d, 1H), 8.51 (s, 1H), 8.73 (s, 1H), 8.91 (s, 1H), 9.57 (bs, 1H); Mass spectrum: 451 (MH+).

### Example 20A

### 4-(5-chloro-4-(imidazo[1,2-a]pyridin-3-yl)pyrimidin-2-ylamino)-3-methoxybenzaldehyde

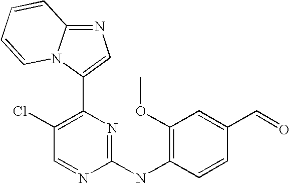

3-(2,5-dichloropyrimidin-4-yl)imidazo[1,2-a]pyridine (see Method 1, 400 mg, 1.51 mmol), 4-amino-3-methoxybenzaldehyde (228 mg, 1.51 mmol), 9,9-dimethyl-4,5-bis(diphenylphosphino)xanthene (87 mg, 0.15 mmol), tris(dibenzylideneacetone)dipalladium (34.5 mg, 0.04 mmol) and 1,8-diazabicyclo[5.4.0]undec-7-ene (0.56 ml, 3.77 mmol) were dissolved in dioxane (12 ml) then the mixture was degassed with argon. The reaction mixture was heated to reflux during 2 hours then filtered and concentrated. The crude product was purified by flash chromatography on silica gel eluting with 0 to 50% ethyl acetate in dichloromethane to afford 4-(5-chloro-4-(imidazo[1,2-a]pyridin-3-yl)pyrimidin-2-ylamino)-3-methoxybenzaldehyde as a clear yellow solid (340 mg, 59%); NMR spectrum: 3.97 (s, 3H), 7.10 (dd, 1H), 7.53-7.61 (m, 3H), 7.81 (d, 1H), 8.29 (d, 1H), 8.69 (s, 1H), 8.75 (s, 1H), 9.08 (s, 1H), 8.78 (d, 1H), 9.92 (s, 1H); Mass spectrum: 380 (MH+).

### Example 21

The following compounds were prepared using a similar procedure to that described in Example 20 using 4-(5-chloro-4-(imidazo[1,2-a]pyridin-3-yl)pyrimidin-2-ylamino)-3-methoxybenzaldehyde (Example 20A) and the appropriate amine as the starting materials. The amines used as the starting materials are indicated in the following table.

| | | Starting | | | % |
|---|---|---|---|---|---|
| Ex. No. | Name | amine | MH+ | NMR spectrum | Yield |
| 21.1 | 2-[[4-[(5-chloro-4- | 2-amino | 425 | 2.61 (t, 2H), 2.48-2.54 (m, | 14 |
| | imidazo[1,2-a]pyridin- | ethanol | | 2H), 3.75 (s, 2H), 3.80 (s, | |
| | 3-yl-pyrimidin-2- | | | 3H), 4.51 (bs, 1H), | |
| | yl)amino]-3-methoxy- | | | 6.94 (dd, 1H), 6.96 (dd, 1H), | |
| | phenyl]methylamino]ethanol | | | 7.14 (d, 1H), 7.47-7.53 (m, | |
| | | | | 2H), 7.77 (ddd, 1H), | |
| | | | | 8.51 (s, 1H), 8.74 (s, 1H), | |
| | | | | 8.90 (s, 1H), 9.58 (bs, 1H) | |
| 21.2 | 2-[[4-[(5-chloro-4- | 2-methyl | 439 | 2.21 (s, 3H), 3.47 (t, 2H), | 71 |
| | imidazo[1,2-a]pyridin- | aminoethanol | | 3.51-3.59 (m, 4H), 3.80 (s, | |
| | 3-yl-pyrimidin-2- | | | 3H), 4.44 (bs, 1H), | |
| | yl)amino]-3-methoxy- | | | 6.92 (dd, 1H), 6.95 (dd, 1H), | |
| | phenyl]methyl-methyl- | | | 7.10 (d, 1H), 7.50 (ddd, | |
| | amino]ethanol | | | 1H), 7.53 (d, 1H), | |
| | | | | 7.77 (ddd, 1H), 8.52 (s, 1H), | |
| | | | | 8.74 (s, 1H), 8.90 (s, 1H), | |
| | | | | 9.60 (bs, 1H) | |
| 21.3 | 3-[[4-[(5-chloro-4- | 3-aminopropan- | 439 | 1.57-1.66 (m, 2H), 2.59 (t, | 66 |
| | imidazo[1,2-a]pyridin- | 1-ol | | 2H), 3.21-3.26 (m, 1H), | |
| | 3-yl-pyrimidin-2- | | | 3.49 (s, 2H), 3.72 (s, 2H), | |
| | yl)amino]-3-methoxy- | | | 3.79 (s, 3H), 6.93 (dd, 1H), | |
| | phenyl]methylamino]propan- | | | 6.95 (dd, 1H), 7.13 (d, | |
| | 1- | | | 1H), 7.46-7.52 (m, 2H), | |
| | ol | | | 7.77 (d, 1H), 8.51 (s, 1H), | |
| | | | | 8.74 (s, 1H), 8.90 (s, 1H), | |
| | | | | 9.58 (bs, 1H) | |
| 21.4 | 5-chloro-4-imidazo[1,2- | 1-methyl | 464 | 2.17 (s, 3H), 2.35 (bs, 4H), | 75 |
| | a]pyridin-3-yl-N-[2- | piperazine | | 2.41 (bs, 4H), 3.49 (s, 2H), | |
| | methoxy-4-[(4-methylpiperazin- | | | 3.78 (s, 3H), 6.88-6.95 (m, | |
| | 1-yl)methyl]phenyl]pyrimidin- | | | 2H), 7.06 (d, 1H), | |
| | 2- | | | 7.47-7.53 (m, 2H), 7.77 (ddd, | |
| | amine | | | 1H), 8.52 (s, 1H), 8.73 (s, | |
| | | | | 1H) 8.93 (s, 1H), 9.57 (bs, | |
| | | | | 1H) | |
| 21.5 | 1-[[4-[(5-chloro-4- | piperidin-4-ol | 465 | 1.36-1.47 (m, 2H), | 58 |
| | imidazo[1,2-a]pyridin- | | | 1.68-1.77 (m, 2H), | |
| | 3-yl-pyrimidin-2- | | | 2.01-2.11 (m, 2H), 2.68-2.76 (m, | |
| | yl)amino]-3-methoxy- | | | 2H), 2.47 (s, 2H), 2.49 (bs, | |
| | phenyl]methyl]piperidin- | | | 1H), 3.79 (s, 3H), 4.56 (d, | |
| | 4-ol | | | 1H), 6.86-6.95 (m, 2H), | |
| | | | | 7.06 (d, 1H), 7.46-7.53 (m, | |
| | | | | 2H), 7.77 (ddd, 1H), | |
| | | | | 8.52 (s, 1H), 8.73 (s, 1H), | |
| | | | | 8.93 (s, 1H), 9.57 (bs, 1H) | |
| 21.6 | 1-[4-[[4-[(5-chloro-4- | 1-piperazin- | 492 | 2.00 (s, 3H), 2.32-2.38 (m, | 83 |
| | imidazo[1,2-a]pyridin- | 1-ylethanone | | 2H), 2.38-2.45 (m, 2H), | |
| | 3-yl-pyrimidin-2- | | | 3.41-3.50 (m, 4H), 3.54 (s, | |
| | yl)amino]-3-methoxy- | | | 2H), 3.80 (s, 3H), 6.99 | |
| | phenyl]methyl]piperazin- | | | 6.96 (m, 2H), 7.08 (d, 1H), | |
| | 1-yl]ethanone | | | 7.51 (ddd, 1H), 7.54 (d, | |
| | | | | 1H), 7.78 (d, 1H), 8.53 (s, | |
| | | | | 1H), 8.76 (s, 1H), 8.93 (s, | |
| | | | | 1H), 9.59 (bs, 1H) | |
| 21.7 | 1-[[4-[(5-chloro-4- | piperidine-4- | 492 | 1.53-1.64 (m, 2H), | 57 |
| | imidazo[1,2-a]pyridin- | carboxamide | | 1.64-1.71 (m, 2H), | |
| | 3-yl-pyrimidin-2- | | | 1.90-1.99 (m, 2H), 2.04-2.13 (m, | |
| | yl)amino]-3-methoxy- | | | 1H), 2.88-2.91 (m, 2H), | |
| | phenyl]methyl]piperidine- | | | 3.47 (s, 2H), 3.79 (s, 3H), | |
| | 4- | | | 6.73 (s, 1H), 6.88-6.96 (m, | |
| | carboxamide | | | 2H), 7.07 (d, 1H), 7.22 (s, | |
| | | | | 1H), 7.49 (ddd, 1H), | |
| | | | | 7.51 (d, 1H), 7.78 (d, 1H), | |
| | | | | 8.52 (s, 1H), 8.74 (s, 1H), | |
| | | | | 8.92 (s, 1H), 9.58 (bs, 1H) | |
| 21.8 | 2-[4-[[4-[(5-chloro-4- | 2-piperazin- | 494 | 2.39 (t, 2H), 2.43 (bs, 8H), | 81 |
| | imidazo[1,2-a]pyridin- | 1-ylethanol | | 3.45-3.53 (m, 4H), 3.79 (s, | |
| | 3-yl-pyrimidin-2- | | | 3H), 4.38 (t, 1H), | |
| | yl)amino]-3-methoxy- | | | 6.87-6.96 (m, 2H), 7.06 (d, 1H), | |
| | phenyl]methyl]piperazin- | | | 7.47-7.54 (m, 2H), 7.77 (d, | |
| | 1-yl]ethanol | | | 1H), 8.52 (s, 1H), 8.73 (s, | |
| | | | | 1H), 8.93 (s, 1H), 9.57 (bs, | |
| | | | | 1H) | |
| 21.9 | 5-chloro-4-imidazo[1,2- | 3-methoxypropan- | 453 | 1.65-1.72 (m, 2H), 2.55 (t, | 59 |
| | a]pyridin-3-yl-N-[2- | 1- | | 2H), 3.21 (s, 3H), 3.38 (t | |
| | methoxy-4-[(3-methoxypropylamino)methyl]phenyl]pyrimidin- | amine | | partially hidden by H2O, | |
| | 2- | | | 2H), 3.72 (s, 2H), 3.79 (s, | |
| | amine | | | 3H), 6.93 (dd, 1H), | |
| | | | | 6.95 (dd, 1H), 7.13 (d, 1H), | |
| | | | | 7.46-7.52 (m, 2H), | |
| | | | | 7.77 (ddd, 1H), 8.51 (s, 1H), | |
| | | | | 8.74 (s, 1H), 8.90 (bs, 1H), | |
| | | | | 9.58 (bs, 1H) | |
| 21.10 | N-[4-(azetidin-1- | azetidine | 421 | 1.97-2.05 (m, 2H), 3.16 (t, | 59 |
| | ylmethyl)-2-methoxy- | | | 4H), 3.55 (s, 2H), 3.79 (s, | |
| | phenyl]-5-chloro-4- | | | 3H), 6.87 (dd, 1H), | |
| | imidazo[1,2-a]pyridin- | | | 6.95 (dd, 1H), 7.03 (d, 1H), | |
| | 3-yl-pyrimidin-2-amine | | | 7.48-7.54 (m, 2H), 7.77 (d, | |
| | | | | 1H), 8.52 (s, 1H), 8.73 (s, | |
| | | | | 1H), 8.89 (bs, 1H), | |
| | | | | 9.57 (bs, 1H) | |
| 21.11 | 5-chloro-4-imidazo[1,2- | 1-methyl-1,4- | 478 | 1.70-1.78 (m, 2H), 2.26 (s, | 52 |
| | a]pyridin-3-yl-N-[2- | diazepane | | 3H), 2.51-2.55 (m partially | |
| | methoxy-4-[(4-methyl- | | | hidden by DMSOd5, 2H), | |
| | 1,4-diazepan-1- | | | 2.56-2.61 (m, 2H), | |
| | yl)methyl]phenyl]pyrimidin- | | | 2.62-2.71 (m, 4H), 3.63 (s, 2H), | |
| | 2- | | | 3.79 (s, 3H), 6.89-6.96 (m, | |
| | amine | | | 2H), 7.09 (d, 1H), | |
| | | | | 7.50 (ddd, 1H), 7.52 (d, 1H), | |
| | | | | 7.77 (ddd, 1H), 8.52 (s, | |
| | | | | 1H), 8.73 (s, 1H), 8.90 (s, | |
| | | | | 1H), 9.59 (bs, 1H), | |
| 21.12 | (3R)-1-[[4-[(5-chloro-4- | (3R)- | 465 | 1.04-1.15 (m, 1H), | 72 |
| | imidazo[1,2-a]pyridin- | piperidin-3-ol | | 1.40-1.51 (m, 1H), | |
| | 3-yl-pyrimidin-2- | | | 1.60-1.67 (m, 1H), 1.72-1.79 (m, | |
| | yl)amino]-3-methoxy- | | | 1H), 1.79-1.86 (m, 1H), | |
| | phenyl]methyl]piperidin- | | | 1.86-1.95 (m, 1H), | |
| | 3-ol | | | 2.65-2.72 (m, 1H), | |
| | | | | 2.81-2.88 (m, 1H), 3.44 (d, 1H), | |
| | | | | 3.47-3.53 (m, 1H), 3.54 (d, | |
| | | | | 1H), 3.79 (s, 3H), 4.60 (d, | |
| | | | | 1H), 6.90 (dd, 1H), | |
| | | | | 6.93 (dd, 1H), 7.07 (s, 1H), | |
| | | | | 6.46-6.54 (m, 2H), 7.77 (d, | |
| | | | | 1H), 8.52 (s, 1H), 8.74 (s, | |
| | | | | 1H), 8.92 (s, 1H), 9.58 (bs, | |
| | | | | 1H) | |
| 21.13 | 5-chloro-4-imidazo[1,2- | 2-methoxy- | 453 | 2.22 (s, 3H), 2.55 (t, 2H), | 58 |
| | a]pyridin-3-yl-N-[2- | N-methyl | | 3.25 (s, 3H), 3.48 (t, 2H), | |
| | methoxy-4-[(2- | ethanamine | | 3.54 (s, 2H), 3.79 (s, 3H), | |
| | methoxyethyl-methyl- | | | 6.91 (dd, 1H), 6.93 (dd, | |
| | amino)methyl]phenyl]pyrimidin- | | | 1H), 7.09 (d, 1H), | |
| | 2-amine | | | 7.50 (ddd, 1H), 7.53 (d, 1H), | |
| | | | | 7.77 (d, 1H), 8.52 (s, 1H), | |
| | | | | 8.73 (s, 1H), 8.91 (s, 1H), | |
| | | | | 9.59 (bs, 1H) | |
| 21.14 | 5-chloro-4-imidazo[1,2- | 1,4- | 465 | 1.79-1.87 (m, 2H), | 65 |
| | a]pyridin-3-yl-N-[2- | oxazepane | | 2.62-2.70 (m, 4H), | |
| | methoxy-4-(1,4- | | | 3.61-3.65 (m, 2H), 3.66 (s, 2H), | |
| | oxazepan-4- | | | 3.70-3.76 (m, 2H), 3.80 (s, | |
| | ylmethyl)phenyl]pyrimidin- | | | 3H), 6.92 (dd, 1H), | |
| | 2-amine | | | 6.94 (dd, 1H), 7.10 (d, 1H), | |
| | | | | 7.50 (ddd, 1H), 7.53 (d, | |
| | | | | 1H), 7.77 (d, 1H), 8.53 (s, | |
| | | | | 1H), 8.73 (s, 1H), 8.91 (s, | |
| | | | | 1H), 9.59 (bs, 1H) | |
| 21.15 | (2R)-2-[[4-[(5-chloro-4- | (2R)-2-aminopropan- | 439 | 0.98 (d, 3H), 2.08 (bs, 1H), | 54 |
| | imidazo[1,2-a]pyridin- | 1-ol | | 2.62-2.71 (m, 1H), | |
| | 3-yl-pyrimidin-2- | | | 3.28-3.38 (m partially hidden by | |
| | yl)amino]-3-methoxy- | | | H2O, 2H), 3.72 (d, 1H), | |
| | phenyl]methylamino]propan- | | | 3.79 (s, 3H), 3.82 (d, 1H), | |
| | 1- | | | 4.56 (t, 1H), 6.94 (dd, 1H), | |
| | ol | | | 6.96 (dd, 1H), 7.14 (d, | |
| | | | | 1H), 7.46-7.53 (m, 2H), | |
| | | | | 7.77 (d, 1H), 8.51 (s, 1H), | |
| | | | | 8.74 (s, 1H), 8.90 (s, 1H), | |
| | | | | 9.58 (bs, 1H) | |
| 21.16 | 1-[[4-[(5-chloro-4- | azetidin-3-ol | 437 | 2.76-2.83 (m, 2H), | 56 |
| | imidazo[1,2-a]pyridin- | | | 3.48-3.55 (m, 2H), 3.58 (s, 2H), | |
| | 3-yl-pyrimidin-2- | | | 3.79 (s, 3H), 4.18-4.27 (m, | |
| | yl)amino]-3-methoxy- | | | 1H), 5.32 (d, 1H), | |
| | phenyl]methyl]azetidin- | | | 6.87 (dd, 1H), 6.95 (dd, 1H), | |
| | 3-ol | | | 7.02 (d, 1H), 7.48-7.55 (m, | |
| | | | | 2H), 7.77 (d, 1H), 8.52 (s, | |
| | | | | 1H), 8.74 (s, 1H), 8.89 (s, | |
| | | | | 1H), 8.58 (bs, 1H) | |
| 21.17 | (3S)-1-[[4-[(5-chloro-4- | (3S)- | 465 | 1.04-1.14 (m, 1H), | 81 |
| | imidazo[1,2-a]pyridin- | piperidin-3-ol | | 1.40-1.51 (m, 1H), | |
| | 3-yl-pyrimidin-2- | | | 1.60-1.67 (m, 1H), 1.71-1.79 (m, | |
| | yl)amino]-3-methoxy- | | | 1H), 1.79-1.85 (m, 1H), | |
| | phenyl]methyl]piperidin- | | | 1.86-1.98 (m, 1H), | |
| | 3-ol | | | 2.66-2.72 (m, 1H), | |
| | | | | 2.81-2.88 (m, 1H), 3.44 (d, 1H), | |
| | | | | 3.46-3.53 (m, 1H), 3.54 (d, | |
| | | | | 1H) 3.79 (s, 3H), 4.60 (d, | |
| | | | | 1H), 6.91 (dd, 1H), | |
| | | | | 6.93 (dd, 1H), 7.07 (d, 1H), | |
| | | | | 7.50 (ddd, 1H), 7.51 (d, | |
| | | | | 1H), 7.77 (ddd, 1H), | |
| | | | | 8.52 (s, 1H), 8.74 (s, 1H), | |
| | | | | 8.93 (s, 1H), 9.59 (bs, 1H) | |
| 21.18 | (3R)-1-[[4-[(5-chloro-4- | (3R)- | 451 | 1.52-1.61 (m, 1H), | 69 |
| | imidazo[1,2-a]pyridin- | pyrrolidin-3- | | 1.98-2.07 (m, 1H), 2.36 (dd, | |
| | 3-yl-pyrimidin-2- | ol | | 1H), 2.41-2.48 (m, 1H), | |
| | yl)amino]-3-methoxy- | | | 2.57-2.65 (m, 1H), | |
| | phenyl]methyl]pyrrolidin- | | | 2.71 (dd, 1H), 3.57 (d, 1H), | |
| | 3-ol | | | 3.62 (d, 1H), 3.79 (s, 3H), | |
| | | | | 4.19-4.26 (m, 1H), 4.72 (d, | |
| | | | | 1H), 6.92 (dd, 1H), | |
| | | | | 6.94 (dd, 1H), 7.07 (d, 1H), | |
| | | | | 7.50 (ddd, 1H), 7.51 (d, | |
| | | | | 1H) 7.77 (d, 1H), 8.52 (s, | |
| | | | | 1H), 8.74 (s, 1H), 8.92 (s, | |
| | | | | 1H), 9.57 (bs, 1H) | |
| 21.19 | (2S)-2-[[4-[(5-chloro-4- | (2S)-2-aminopropan- | 439 | 0.98 (d, 3H), 2.08 (bs, 1H), | 51 |
| | imidazo[1,2-a]pyridin- | 1-ol | | 2.62-2.71 (m, 1H), | |
| | 3-yl-pyrimidin-2- | | | 3.27-3.37 (m partially hidden by | |
| | yl)amino]-3-methoxy- | | | H2O, 2H), 3.72 (d, 1H), | |
| | phenyl]methylamino]propan- | | | 3.79 (s, 3H), 3.82 (d, 1H), | |
| | 1- | | | 4.51 (t, 1H), 6.94 (dd, 1H), | |
| | ol | | | 6.96 (dd, 1H), 7.14 (d, | |
| | | | | 1H), 7.46-7.53 (m, 2H), | |
| | | | | 7.77 (d, 1H), 8.52 (s, 1H), | |
| | | | | 8.74 (s, 1H), 8.90 (s, 1H), | |
| | | | | 9.58 (bs, 1H) | |
| 21.20 | 5-chloro-4-imidazo[1,2- | morpholine | 451 | 2.34-2.43 (m, 4H), 3.49 (s, | 32 |
| | a]pyridin-3-yl-N-[2- | | | 2H), 3.56-3.64 (m, 4H), | |
| | methoxy-4- | | | 3.79 (s, 3H), 6.88-6.95 (m, | |
| | (morpholinomethyl)phenyl]pyrimidin- | | | 2H), 7.07 (d, 1H), | |
| | 2- | | | 7.58-7.55 (m, 2H), 7.76 (d, 1H), | |
| | amine | | | .52 (s, 1H), 8.72 (s, 1H), | |
| | | | | 8.93 (s, 1H), 9.57 (bs, 1H) | |

### Example 22

### [4-[4-[(5-chloro-4-imidazo[1,2-a]pyridin-3-yl-pyrimidin-2-yl)amino]-3-methoxy-phenyl]piperazin-1-yl]-(1-hydroxycyclopropyl)methanone

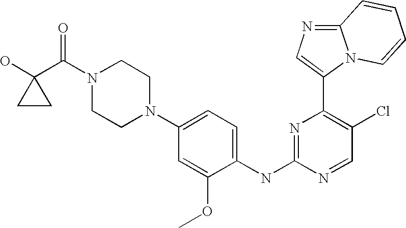

A mixture of 5-chloro-4-(imidazo[1,2-a]pyridin-3-yl)-N-(2-methoxy-4-(piperazin-1-yl)phenyl)pyrimidin-2-amine (Example 2, 70 mg, 0.16 mmol), N,N-diisopropylethylamine (0.056 mL, 0.32 mmol), 2-(3H-[1,2,3]triazolo[4,5-b]pyridin-3-yl)-1,1,3,3-tetramethylisouronium hexafluorophosphate (122 mg, 0.32 mmol) and 1-hydroxycyclopropanecarboxylic acid (33 mg, 0.32 mmol) in dichloromethane (1 mL) was stirred at 40° C. for 3 hours. The volatiles were evaporated and the residue was purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent. The fractions containing the desired compound were evaporated to dryness to afford (4-(4-(5-chloro-4-(imidazo[1,2-a]pyridin-3-yl)pyrimidin-2-ylamino)-3-methoxyphenyl)piperazin-1-yl)(1-hydroxycyclopropyl)methanone (18 mg, 21%) as a pale yellow solid; NMR spectrum: 0.76-0.83 (m, 2H), 0.93-0.99 (m, 2H), 3.21 (bs, 4H), 3.77 (s, 3H), 3.80 (bs, 4H), 6.39 (s, 1H), 6.58 (dd, 1H), 6.75 (d, 1H), 6.90 (bs, 1H), 7.30 (d, 1H), 7.49 (ddd, 1H), 7.75 (d, 1H), 8.46 (s, 1H), 8.72 (s, 1H), 8.80 (s, 1H), 9.56 (bs, 1H); Mass spectrum: 520 (MH+).

### Example 23

### [4-[4-[(5-chloro-4-imidazo[1,2-a]pyridin-3-yl-pyrimidin-2-yl)amino]-3-methoxy-phenyl]piperazin-1-yl]-[(2S)-pyrrolidin-2-yl]methanone

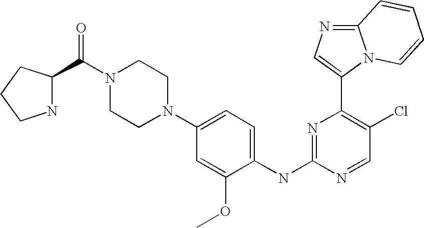

A mixture of 5-chloro-4-(imidazo[1,2-a]pyridin-3-yl)-N-(2-methoxy-4-(piperazin-1-yl)phenyl)pyrimidin-2-amine (Example 2, 122 mg, 0.28 mmol), (S)-1-(tert-butoxycarbonyl)pyrrolidine-2-carboxylic acid (90 mg, 0.42 mmol), 2-(3H-[1,2,3]triazolo[4,5-b]pyridin-3-yl)-1,1,3,3-tetramethylisouronium hexafluorophosphate (213 mg, 0.56 mmol) and N,N-diisopropylethylamine (0.097 mL, 0.56 mmol) in dichloromethane (2 mL) was stirred at 25° C. for 5 hours. Trifluoroacetic acid (2 mL) was added and the reaction mixture was stirred at 25° C. for 10 hours. The volatiles were evaporated and the residue was purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent. The fractions containing the desired compound were evaporated to dryness to afford (4-(4-(5-chloro-4-(imidazo[1,2-a]pyridin-3-yl)pyrimidin-2-ylamino)-3-methoxyphenyl)piperazin-1-yl)((S)-pyrrolidin-2-yl)methanone (70 mg, 47%) as a yellow solid; NMR spectrum: 1.55-1.68 (m, 2H), 1.68-1.75 (m, 1H), 2.01-2.10 (m, 1H), 2.66-2.74 (m, 1H), 3.00-3.07 (m, 1H), 3.13-3.26 (m, 4H), 3.60-3.67 (m, 2H), 3.67-3.74 (m, 2H), 3.77 (s, 3H), 3.93-3.99 (m, 1H), 6.57 (dd, 1H), 6.75 (d, 1H), 6.89 (bs, 1H), 7.31 (d, 1H), 7.49 (dd, 1H), 7.75 (d, 1H), 8.46 (s, 1H), 8.72 (s, 1H), 8.81 (s, 1H), 9.56 (bs, 1H); Mass spectrum: 533 (MH+).

### Example 24

The following compounds were prepared using a similar procedure to that described in Example 23 using 5-chloro-4-(imidazo[1,2-a]pyridin-3-yl)-N-(2-methoxy-4-(piperazin-1-yl)phenyl)pyrimidin-2-amine and the appropriate protected alpha-amino acid as the starting materials. The protected alpha-amino acids used as the starting material are indicated in the following table.

| | | | | | % |
|---|---|---|---|---|---|
| Ex. No. | Name | Starting acid | MH+ | NMR spectrum | Yield |
| 24.1 | [4-[4-[(5-chloro-4- | (R)-1-(tert- | 547 | 1.32-1.41 (m, 2H), | 34 |
| | imidazo[1,2- | butoxycarbonyl)piperidine- | | 1.51-1.58 (m, 2H), | |
| | a]pyridin-3-yl- | 2- | | 1.70-1.77 (m, 1H), 1.77-1.84 (m, | |
| | pyrimidin-2-yl)amino]- | carboxylic acid | | 1H), 2.58-2.68 (m, 1H), | |
| | 3-methoxy- | | | 3.01-3.09 (m, 1H), | |
| | phenyl]piperazin-1- | | | 3.10-3.27 (m, 4H), | |
| | yl]-[(2R)-2- | | | 3.56-3.76 (m, 4H), 3.77 (s, 3H), | |
| | piperidyl]methanone | | | 3.78-3.85 (m, 1H), | |
| | | | | 6.57 (dd, 1H), 6.74 (d, 1H), | |
| | | | | 6.90 (bs, 1H), 7.31 (d, 1H), | |
| | | | | 7.49 (ddd, 1H), 7.76 (d, | |
| | | | | 1H), 8.46 (s, 1H), 8.72 (s, | |
| | | | | 1H), 8.81 (s, 1H), 9.56 (bs, | |
| | | | | 1H) | |
| 24.2 | [4-[4-[(5-chloro-4- | (S)-1-(tert- | 547 | 1.32-1.41 (m, 2H), | 41 |
| | imidazo[1,2- | butoxycarbonyl)piperidine- | | 1.51-1.58 (m, 2H), | |
| | a]pyridin-3-yl- | 2- | | 1.70-1.77 (m, 1H), 1.77-1.84 (m, | |
| | pyrimidin-2-yl)amino]- | carboxylic acid | | 1H), 2.58-2.68 (m, 1H), | |
| | 3-methoxy- | | | 3.01-3.09 (m, 1H), | |
| | phenyl]piperazin-1- | | | 3.10-3.27 (m, 4H), | |
| | yl]-[(2S)-2-piperidyl]methanone | | | 3.56-3.76 (m, 4H), 3.77 (s, 3H), | |
| | | | | 3.78-3.85 (m, 1H), | |
| | | | | 6.57 (dd, 1H), 6.75 (d, 1H), | |
| | | | | 6.90 (bs, 1H), 7.31 (d, 1H), | |
| | | | | 7.49 (ddd, 1H), 7.75 (d, | |
| | | | | 1H), 8.46 (s, 1H), 8.72 (s, | |
| | | | | 1H), 8.81 (s, 1H), 9.56 (bs, | |
| | | | | 1H) | |
| 24.3 | [4-[4-[(5-chloro-4- | 1-(tert- | 533 | 1.58-1.69 (m, 2H), | 28 |
| | imidazo[1,2- | butoxycarbonyl)pyrrolidine- | | 1.69-1.76 (m, 1H), | |
| | a]pyridin-3- | 2- | | 2.03-2.10 (m, 1H), 2.67-2.74 (m, | |
| | ylpyrimidin-2- | carboxylic acid | | 1H), 3.00-3.07 (m, 1H), | |
| | yl)amino]-3- | | | 3.10-3.25 (m, 4H), | |
| | methoxyphenyl]piperazin- | | | 3.59-3.75 (m, 4H), (m, 1H), | |
| | 1-yl]-pyrrolidin- | | | 3.77 (s, 3H), 3.94-4.00 (m, | |
| | 2-yl methanone | | | 1H), 6.57 (dd, 1H), | |
| | | | | 6.75 (d, 1H), 6.90 (bs, 1H), | |
| | | | | 7.31 (d, 1H), 7.49 (ddd, 1H), | |
| | | | | 7.76 (d, 1H), 8.46 (s, 1H), | |
| | | | | 8.72 (s, 1H), 8.81 (s, 1H), | |
| | | | | 9.56 (bs, 1H) | |
| 24.4 | 2-amino-1-[4-[4-[(5- | N-(tert-butoxycarbonyl)glycine | 493 | 1.64 (bs, 2H), | 24 |
| | chloro-4- | | | 3.14-3.24 (m, 4H), 3.39 (s, 2H), | |
| | imidazo[1,2- | | | 3.50-3.59 (m, 2H), | |
| | a]pyridin-3-yl- | | | 3.61-3.70 (m, 2H), 3.77 (s, 3H), | |
| | pyrimidin-2- | | | 6.56 (dd, 1H), 6.74 (d, | |
| | yl)amino]-3- | | | 1H), 6.89 (bs, 1H), 7.30 (d, | |
| | methoxy-phenyl]piperazin- | | | 1H), 7.49 (dd, 1H), | |
| | 1-yl]ethanone | | | 7.75 (d, 1H), 8.46 (s, 1H), | |
| | | | | 8.72 (s, 1H), 8.80 (s, 1H), | |
| | | | | 9.55 (bs, 1H) | |
| 24.5 | (2S)-2-amino-1-[4- | N-(tert-butoxycarbonyl)- | 507 | 1.11 (d, 3H), 1.71 (bs, 2H), | 32 |
| | [4-[(5-chloro-4- | L- | | 3.12-3.25 (m, 4H), | |
| | imidazo[1,2- | alanine | | 3.59-3.73 (m, 4H), 3.77 (s, 3H), | |
| | a]pyridin-3-yl- | | | 3.80 (q, 1H), 6.56 (dd, | |
| | pyrimidin-2-yl)amino]- | | | 1H), 6.74 (d, 1H), 6.89 (bs, | |
| | 3-methoxy- | | | 1H), 7.30 (d, 1H), | |
| | phenyl]piperazin-1- | | | 7.49 (dd, 1H), 7.75 (d, 1H), | |
| | yl]propan-1-one | | | 8.46 (s, 1H), 8.72 (s, 1H), | |
| | | | | 8.80 (s, 1H), 9.55 (bs, 1H) | |
| 24.6 | (2S)-2-amino-1-[4- | N-(tert-butoxycarbonyl)- | 535 | 0.82 (d, 3H), 0.91 (d, 3H), | 26 |
| | [4-[(5-chloro-4- | L- | | 1.57 (bs, 2H), | |
| | imidazo[1,2- | valine | | 1.70-1.79 (m, 1H), 3.12-3.26 (m, | |
| | a]pyridin-3-yl- | | | 4H), 3.50 (d, 1H), | |
| | pyrimidin-2- | | | 3.61-3.74 (m, 4H), 3.77 (s, 3H), | |
| | yl)amino]-3- | | | 6.56 (dd, 1H), 6.74 (d, | |
| | methoxy-phenyl]piperazin- | | | 1H), 6.89 (bs, 1H), 7.31 (d, | |
| | 1-yl]-3- | | | 1H), 7.48 (dd, 1H), | |
| | methyl-butan-1-one | | | 7.75 (d, 1H), 8.46 (s, 1H), | |
| | | | | 8.72 (s, 1H), 8.81 (s, 1H), | |
| | | | | 9.55 (bs, 1H) | |
| 24.7 | (2S)-2-amino-1-[4- | N-(tert-butoxycarbonyl)- | 523 | 1.72 (bs, 2H), | 29 |
| | [4-[(5-chloro-4- | L- | | 3.13-3.27 (m, 4H), 3.42-3.50 (m, | |
| | imidazo[1,2- | serine | | 1H), 3.61-3.81 (m, 6H), | |
| | a]pyridin-3-yl- | | | 3.77 (s, 3H), 4.72 (t, 1H), | |
| | pyrimidin-2- | | | 6.56 (dd, 1H), 6.74 (d, | |
| | yl)amino]-3- | | | 1H), 6.89 (bs, 1H), 7.30 (d, | |
| | methoxy-phenyl]piperazin- | | | 1H), 7.49 (dd, 1H), | |
| | 1-yl]-3- | | | 7.75 (d, 1H), 8.46 (s, 1H), | |
| | hydroxy-propan-1- | | | 8.72 (s, 1H), 8.80 (s, 1H), | |
| | one | | | 9.55 (bs, 1H) | |
| 24.8 | (3S)-3-amino-4-[4- | N-α-(tert- | 550 | 2.15 (dd, 1H), 2.35 (dd, | 12 |
| | [4-[(5-chloro-4- | butoxycarbonyl)- | | 1H), 3.17 (bs, 2H), | |
| | imidazo[1,2- | L- | | 3.24 (bs, 2H), 3.58-3.82 (m, | |
| | a]pyridin-3-yl- | asparagine | | 4H), 3.77 (s, 3H), 4.04 (dd, | |
| | pyrimidin-2- | | | 1H), 6.56 (dd, 1H), | |
| | yl)amino]-3- | | | 6.75 (d, 1H), 6.83 (bs, 1H), | |
| | methoxy-phenyl]piperazin- | | | 6.90 (bs, 1H), 7.30 (d, 1H), | |
| | 1-yl]-4- | | | 7.39 (bs, 1H), 7.50 (dd, 1H), | |
| | oxo-butanamide | | | 7.75 (d, 1H), 8.46 (s, 1H), | |
| | | | | 8.72 (s, 1H), 8.80 (s, 1H), | |
| | | | | 9.55 (bs, 1H) | |
| 24.9 | [4-[4-[(5-chloro-4- | (2S,4R)-4- | 549 | 1.75-1.90 (m, 2H), | 36 |
| | imidazo[1,2- | hydroxy-1-[(2- | | 2.55 (dd, 1H), 3.07 (dd, 1H), | |
| | a]pyridin-3-yl- | methylpropan- | | 3.13-3.26 (m, 4H), | |
| | pyrimidin-2-yl)amino]- | 2-yl)oxycarbonyl]pyrrolidine- | | 3.59-3.74 (m, 4H), 3.77 (s, 3H), | |
| | 3-methoxy- | 2- | | 4.02-4.09 (m, 1H), | |
| | phenyl]piperazin-1- | carboxylic acid | | 4.16-4.22 (m, 1H), 4.72 (d, 1H), | |
| | yl]-[(2S,4R)-4- | | | 6.56 (dd, 1H), 6.75 (d, | |
| | hydroxy pyrrolidin- | | | 1H), 6.89 (bs, 1H), 7.30 (d, | |
| | 2-yl]methanone | | | 1H), 7.49 (dd, 1H), | |
| | | | | 7.75 (d, 1H), 8.46 (s, 1H), | |
| | | | | 8.72 (s, 1H), 8.81 (s, 1H), | |
| | | | | 9.55 (bs, 1H) | |
| 24.10 | 1-[4-[4-[(5-chloro-4- | N-(tert-butoxycarbonyl)- | 507 | 1.93 (bs, 1H), 2.29 (s, 3H), | 44 |
| | imidazo[1,2- | sarcosine | | 3.14-3.24 (m, 4H), 3.37 (s, | |
| | a]pyridin-3-yl- | | | 2H), 3.56-3.62 (m, 2H), | |
| | pyrimidin-2-yl)amino]- | | | 3.62-3.68 (m, 2H), 3.77 (s, | |
| | 3-methoxy- | | | 3H), 6.56 (dd, 1H), | |
| | phenyl]piperazin-1- | | | 6.74 (d, 1H), 6.90 (bs, 1H), | |
| | yl]-2-methylamino- | | | 7.30 (d, 1H), 7.49 (dd, 1H), | |
| | ethanone | | | 7.75 (d, 1H), 8.46 (s, 1H), | |
| | | | | 8.72 (s, 1H), 8.80 (s, 1H), | |
| | | | | 9.55 (bs, 1H) | |
| 24.11 | 1-[4-[4-[(5-chloro-4- | N-(tert-butoxycarbonyl)- | 521 | 1.02 (t, 3H), 1.87 (bs, 1H), | 41 |
| | imidazo[1,2- | N- | | 2.54 (q partially hidden by | |
| | a]pyridin-3-yl- | ethylglycine | | DMSOd5. 2H), | |
| | pyrimidin-2-yl)amino]- | | | 3.13-3.24 (m, 4H), 3.41 (s, 2H), | |
| | 3-methoxy- | | | 3.56-3.62 (m, 2H), | |
| | phenyl]piperazin-1- | | | 3.62-3.68 (m, 2H), 3.77 (s, 3H), | |
| | yl]-2-ethylamino- | | | 6.56 (dd, 1H), 6.74 (d, | |
| | ethanone | | | 1H), 6.90 (bs, 1H), 7.30 (d, | |
| | | | | 1H), 7.49 (dd, 1H), | |
| | | | | 7.75 (d, 1H), 8.46 (s, 1H), | |
| | | | | 8.72 (s, 1H), 8.80 (s, 1H), | |
| | | | | 9.55 (bs, 1H) | |
| 24.12 | (2R)-2-amino-1-[4- | N-(tert-butoxycarbonyl)- | 523 | 1.72 (bs, 2H), | 29 |
| | [4-[(5-chloro-4- | D- | | 3.13-3.27 (m, 4H), 3.42-3.50 (m, | |
| | imidazo[1,2- | serine | | 1H), 3.61-3.81 (m, 6H), | |
| | a]pyridin-3-yl- | | | 3.77 (s, 3H), 4.72 (t, 1H), | |
| | pyrimidin-2- | | | 6.56 (dd, 1H), 6.74 (d, | |
| | yl)amino]-3- | | | 1H), 6.89 (bs, 1H), 7.30 (d, | |
| | methoxy-phenyl]piperazin- | | | 1H), 7.49 (dd, 1H), | |
| | 1-yl]-3- | | | 7.75 (d, 1H), 8.46 (s, 1H), | |
| | hydroxy-propan-1- | | | 8.72 (s, 1H), 8.80 (s, 1H), | |
| | one | | | 9.55 (bs, 1H) | |
| 24.13 | (2S)-2-amino-1-[4- | (S)-2-(tert- | 537 | 1.32-1.43 (m, 1H), | 30 |
| | [4-[(5-chloro-4- | butoxycarbonylamino)- | | 1.60-1.71 (m, 1H), 1.75 (bs, | |
| | imidazo[1,2- | 4- | | 1H), 3.13-3.28 (m, 3H), | |
| | a]pynidin-3-yl- | hydroxy | | 3.47-3.55 (m, 1H), | |
| | pyrimidin-2- | butanoic acid | | 3.55-3.63 (m, 1H), | |
| | yl)amino]-3- | | | 3.63-3.73 (m, 4H), 3.76 (s, 3H), | |
| | methoxy-phenyl]piperazin- | | | 3.83 (dd, 1H), 4.68 (bs, 1H), | |
| | 1-yl]-4- | | | 6.57 (dd, 1H), 6.75 (d, | |
| | hydroxy-butan-1-one | | | 1H), 6.90 (bs, 1H), 7.31 (d, | |
| | | | | 1H), 7.49 (dd, 1H), | |
| | | | | 7.75 (d, 1H), 8.46 (s, 1H), | |
| | | | | 8.72 (s, 1H), 8.80 (s, 1H), | |
| | | | | 9.56 (bs, 1H) | |
| 24.14 | (2R)-2-amino-1-[4- | N-(tert-butoxycarbonyl)- | 507 | 1.16 (d, 3H), 3.09-3.26 (m, | 60 |
| | [4-[(5-chloro-4- | D- | | 4H), 3.54 (bs, 2H), | |
| | imidazo[1,2- | alanine | | 3.57-3.75 (m, 4H), 3.77 (s, 3H), | |
| | a]pyridin-3-yl- | | | 3.95 (q, 1H), 6.57 (dd, | |
| | pyrimidin-2- | | | 1H), 6.75 (d, 1H), 6.90 (bs, | |
| | yl)amino]-3- | | | 1H), 7.31 (d, 1H), | |
| | methoxy-phenyl]piperazin- | | | 7.49 (dd, 1H), 7.76 (d, 1H), | |
| | 1- | | | 8.46 (s, 1H), 8.72 (s, 1H), | |
| | yl]propan-1-one | | | 8.81 (s, 1H), 9.56 (bs, 1H) | |

### Example 25

The following compounds were prepared using a similar procedure to that described in Example 22 using 5-chloro-4-(imidazo[1,2-a]pyridin-3-yl)-N-(2-methoxy-4-(piperazin-1-yl)phenyl)pyrimidin-2-amine and the appropriate acid as the starting materials. The acids used as the starting material are indicated in the following table.

| | | Starting | | | % |
|---|---|---|---|---|---|
| Ex. No. | Name | acid | MH+ | NMR spectrum | Yield |
| 25.1 | 5-[4-[4-[(5-chloro-4- | 5-oxopyrrolidine- | 547 | 1.86-2.21 (m, 4H), | 76 |
| | imidazo[1,2-a]pyridin- | 2-carboxylic | | 2.30-2.46 (m, 1H), | |
| | 3-yl-pyrimidin-2- | acid^{a} | | 3.09-3.30 (m, 4H), 3.57-3.76 (m, | |
| | yl)amino]-3-methoxy- | | | 4H), 3.78 (s, 3H), | |
| | phenyl]piperazine-1- | | | 4.62 (dd, 1H), 6.56 (dd, 1H), | |
| | carbonyl]pyrrolidin-2- | | | 6.76 (d, 1H), 6.90 (bs, | |
| | one | | | 1H), 7.33 (d, 1H), | |
| | | | | 7.49 (ddd, 1H), 7.73-7.79 (m, | |
| | | | | 2H), 8.46 (s, 1H), 8.73 (s, | |
| | | | | 1H), 8.81 (s, 1H), | |
| | | | | 9.56 (bs, 1H) | |
| 25.2 | N-[2-[4-[4-[(5-chloro-4- | 2-acetamido | 535 | 1.89 (s, 3H), | 43 |
| | imidazo[1,2-a]pyridin- | acetic acid | | 3.15-3.20 (m, 2H), 3.20-3.26 (m, | |
| | 3-yl-pyrimidin-2- | | | 2H), 3.58-3.67 (m, 4H), | |
| | yl)amino]-3-methoxy- | | | 3.77 (s, 3H), 4.01 (d, | |
| | phenyl]piperazin-1-yl]- | | | 2H), 6.56 (dd, 1H), | |
| | 2-oxo-ethyl]acetamide | | | 6.75 (d, 1H), 6.89 (bs, 1H), | |
| | | | | 7.30 (d, 1H), 7.50 (ddd, | |
| | | | | 1H), 7.75 (d, 1H), 8.01 (t, | |
| | | | | 1H), 8.46 (s, 1H), 8.72 (s, | |
| | | | | 1H), 8.81 (s, 1H), | |
| | | | | 9.55 (bs, 1H) | |
| 25.3 | (2S)-1-[4-[4-[(5-chloro- | (S)-2- | 522 | 1.26 (d, 3H), | 63 |
| | 4-imidazo[1,2- | methoxy | | 3.15-3.26 (m, 4H), 3.24 (s, 3H), | |
| | a]pyridin-3-yl- | propanoic | | 3.63-3.69 (m, 2H), | |
| | pyrimidin-2-yl)amino]- | acid | | 3.70-3.76 (m, 2H), 3.77 (s, | |
| | 3-methoxy-phenyl]piperazin- | | | 3H), 4.28 (q, 1H), | |
| | 1-yl]-2- | | | 6.57 (dd, 1H), 6.75 (d, 1H), | |
| | methoxy-propan-1-one | | | 6.90 (m, 1H), 7.31 (d, | |
| | | | | 1H), 7.49 (ddd, 1H), | |
| | | | | 7.75 (d, 1H), 8.46 (s, 1H), | |
| | | | | 8.72 (s, 1H), 8.80 (s, 1H), | |
| | | | | 9.56 (bs, 1H) | |
| 25.4 | (2R)-1-[4-[4-[(5-chloro- | (R)-2- | 508 | 1.22 (d, 3H), | 49 |
| | 4-imidazo[1,2- | hydroxy | | 3.10-3.27 (m, 4H), 3.56-3.77 (m, | |
| | a]pyridin-3-yl- | propanoic | | 4H), 3.77 (s, 3H), | |
| | pyrimidin-2-yl)amino]- | acid | | 4.45-4.55 (m, 1H), 4.99 (d, | |
| | 3-methoxy- | | | 1H), 6.57 (dd, 1H), | |
| | phenyl]piperazin-1-yl]- | | | 6.75 (d, 1H), 6.90 (m, 1H), | |
| | 2-hydroxy-propan-1- | | | 7.30 (d, 1H), 7.49 (ddd, | |
| | one | | | 1H), 7.76 (d, 1H), | |
| | | | | 8.46 (s, 1H), 8.72 (s, 1H), | |
| | | | | 8.81 (s, 1H), 9.55 (bs, 1H) | |
| 25.5 | (2R)-1-[4-[4-[(5-chloro- | (R)-2- | 522 | 1.26 (d, 3H), | 79 |
| | 4-imidazo[1,2- | methoxy | | 3.14-3.23 (m, 4H), 3.24 (s, 3H), | |
| | a]pyridin-3-yl- | propanoic | | 3.63-3.70 (m, 2H), | |
| | pyrimidin-2-yl)amino]- | acid | | 3.70-3.76 (m, 2H), 3.77 (s, | |
| | 3-methoxy- | | | 3H), 4.28 (q, 1H), | |
| | phenyl]piperazin-1-yl]- | | | 6.57 (dd, 1H), 6.75 (d, 1H), | |
| | 2-methoxy-propan-1- | | | 6.90 (bs, 1H), 7.31 (d, | |
| | one | | | 1H), 7.49 (dd, 1H), | |
| | | | | 7.75 (d, 1H), 8.46 (s, 1H), | |
| | | | | 8.72 (s, 1H), 8.81 (s, 1H), | |
| | | | | 9.56 (bs, 1H) | |
| 25.6 | 1-[4-[4-[(5-chloro-4- | 2-hydroxy- | 522 | 1.35 (s, 6H), | 22 |
| | imidazo[1,2-a]pyridin- | 2-methyl- | | 3.17-3.24 (m, 4H), 3.58-3.64 (m, | |
| | 3-yl-pyrimidin-2- | propanoic | | 4H), 3.77 (s, 3H), 5.48 (s, | |
| | yl)amino]-3-methoxy- | acid | | 1H), 6.56 (d, 1H), | |
| | phenyl]piperazin-1-yl]- | | | 6.74 (s, 1H), 6.89 (bs, 1H), | |
| | 2-hydroxy-2-methyl- | | | 7.30 (d, 1H), 7.59 (dd, | |
| | propan-1-one | | | 1H), 7.75 (d, 1H), | |
| | | | | 8.46 (s, 1H), 8.72 (s, 1H), | |
| | | | | 8.80 (s, 1H), 9.56 (bs, 1H) | |

Notes:
^{a}enantiomerically pure (5S)-5-[4-[4-[(5-chloro-4-imidazo[1,2-a]pyridin-3-yl-pyrimidin-2-yl)amino]-3-methoxy-phenyl]piperazine-1-carbonyl]pyrrolidin-2-one (Example 25A) was also prepared using (S)-5-oxopyrrolidine-2-carboxylic acid as a starting material (66%).

### Example 26

### N-[4-(azetidin-3-yloxy)-2-methoxy-phenyl]-5-chloro-4-imidazo[1,2-a]pyridin-3-yl-pyrimidin-2-amine

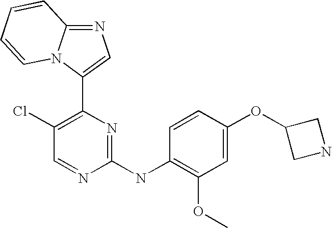

A mixture of tert-butyl 3-(4-amino-3-methoxyphenoxy)azetidine-1-carboxylate (see Method 28, 133 mg, 0.45 mmol), 3-(2,5-dichloropyrimidin-4-yl)imidazo[1,2-a]pyridine (see Method 1, 120 mg, 0.45 mmol), 9,9-dimethyl-4,5-bis(diphenylphosphino)xanthene (26 mg, 0.05 mmol) and tris(dibenzylideneacetone)dipalladium (10.3 mg, 0.01 mmol) and 1,8-diazabicyclo[5.4.0]undec-7-ene (0.169 ml, 1.13 mmol) in dioxane (1.5 ml) was purged with argon and placed into a sealed microwave tube. The reaction was heated in a microwave reactor at 140° C. for 15 minutes then the mixture was diluted with EtOAc and filtered through a plug of celite. The filtrate was evaporated to dryness, dissolved in dichloromethane (2 ml) and treated with TFA (2 ml). The reaction mixture was stirred for one hour at room temperature, solvents were evaporated and the residue was dissolved in dichloromethane (2 ml) and treated with 6N NH3 in MeOH (2 ml). After evaporation, the crude mixture was purified by preparative HPLC using a Waters SunFire reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent to afford N-(4-(azetidin-3-yloxy)-2-methoxyphenyl)-5-chloro-4-(imidazo[1,2-a]pyridin-3-yl)pyrimidin-2-amine (30 mg, 15%) as a yellow solid; NMR spectrum: 1.53 (bs, 1H), 3.51-3.57 (m, 2H), 3.75 (s, 3H), 3.77-3.81 (m, 2H), 4.99-5.07 (m, H), 6.38 (dd, 1H), 6.62 (d, 1H), 6.89 (bs, 1H), 7.32 (d, 1H), 7.50 (ddd, 1H), 7.76 (d, 1H), 8.46 (s, 1H), 87.72 (s, 1H), 8.86 (s, 1H), 9.52 (bs, 1H); Mass spectrum: 423 (MH+).

### Example 27

The following compounds were prepared using a similar procedure to that described in Example 22 using N-[4-(azetidin-3-yloxy)-2-methoxy-phenyl]-5-chloro-4-imidazo[1,2-a]pyridin-3-yl-pyrimidin-2-amine (Example 26) and the appropriate acid as the starting materials. The acids used as the starting material are indicated in the following table.

| | | | | | % |
|---|---|---|---|---|---|
| Ex. No. | Name | Starting acid | MH+ | NMR spectrum | Yield |
| 27.1 | (2S)-1-[3-[4-[(5-chloro- | (S)-2- | 495 | 1.20 (d, 3H), 3.77 (s, | 41 |
| | 4-imidazo[1,2-a]pyridin- | hydroxy | | 3H), 3.80-3.88 (m, | |
| | 3-yl-pyrimidin-2- | propanoic | | 1H), 4.11-4.19 (m, | |
| | yl)amino]-3-methoxy- | acid | | 1H), 4.19-4.26 (m, | |
| | phenoxy]azetidin-1-yl]-2- | | | 1H), 4.36 (ddd, 1H), | |
| | hydroxy-propan-1-one | | | 4.75 (ddd, 1H), | |
| | | | | 5.07-5.14 (m, 1H), 5.16 (d, | |
| | | | | 0.5H), 5.19 (d, 0.5H), | |
| | | | | 6.44 (dd, 1H), 6.67 (s, | |
| | | | | 1H), 6.91 (bs, 1H), | |
| | | | | 7.37 (d, 1H), | |
| | | | | 7.49 (ddd, 1H), | |
| | | | | 7.76 (d, 1H), 8.47 (s, 1H), | |
| | | | | 8.72 (s, 1H), 8.89 (s, | |
| | | | | 1H), 9.54 (bs, 1H) | |
| 27.2 | (2S)-1-[3-[4-[(5-chloro- | (S)-2- | 509 | 1.22 (d, 1.5H), | 82 |
| | 4-imidazo[1,2-a]pyridin- | methoxy | | 1.24 (d, 1.5H), 3.24 (s, 3H, | |
| | 3-yl-pyrimidin-2- | propanoic | | 3.77 (s, 3H), | |
| | yl)amino]-3-methoxy- | acid | | 3.84-3.91 (m, 2H), 4.15 (dd, | |
| | phenoxy]azetidin-1-yl]-2- | | | 0.5H), 4.20 (dd, 0.5H), | |
| | methoxy-propan-1-one | | | 4.38 (dd, 0.5H), | |
| | | | | 4.42 (dd, 0.5H), 4.69 (dd, | |
| | | | | 0.5H), 4.77 (dd, 0.5H), | |
| | | | | 5.10-5.17 (m, 1H), | |
| | | | | 6.45 (dd, 1H), | |
| | | | | 6.67 (dd, 1H), 6.92 (bs, | |
| | | | | 1H), 7.38 (d, 1H), | |
| | | | | 7.49 (dd, 1H), 7.76 (d, 1H), | |
| | | | | 8.48 (s, 1H), 8.72 (s, | |
| | | | | 1H), 8.89 (s, 1H), | |
| | | | | 9.54 (bs, 1H) | |
| 27.3 | 1-[3-[4-[(5-chloro-4- | 2-hydroxy | 481 | 3.77 (s, 3H), 3.86 (dd, | 32 |
| | imidazo[1,2-a]pyridin-3- | acetic acid | | 1H), 3.96 (d, 3H), | |
| | yl-pyrimidin-2- | | | 4.16 (dd, 1H), 4.38 (dd, | |
| | yl)amino]-3-methoxy- | | | 1H), 4.66 (ddd, 1H), | |
| | phenoxy]azetidin-1-yl]-2- | | | 5.02 (t, 1H), | |
| | hydroxy-ethanone | | | 5.09-5.16 (m, 1H), 6.43 (dd, | |
| | | | | 1H), 6.68 (dd, 1H), | |
| | | | | 6.92 (bs, 1H), 7.38 (d, | |
| | | | | 1H), 7.49 (dd, 1H), | |
| | | | | 7.75 (d, 1H), 8.47 (s, | |
| | | | | 1H), 8.72 (s, 1H), | |
| | | | | 8.89 (s, 1H), 9.54 (bs, 1H) | |
| 27.4 | (2R)-1-[3-[4-[(5-chloro- | (R)-2- | 509 | 1.22 (d, 1.5H), | 86 |
| | 4-imidazo[1,2-a]pyridin- | methoxy | | 1.24 (d, 1.5H),, 24 (s, 3H, | |
| | 3-yl-pyrimidin-2- | propanoic | | 3.77 (s, 3H), | |
| | yl)amino]-3-methoxy- | acid | | 3.84-3.91 (m, 2H), 4.15 (dd, | |
| | phenoxy]azetidin-1-yl]-2- | | | 0.5H), 4.20 (dd, 0.5H), | |
| | methoxy-propan-1-one | | | 4.38 (dd, 0.5H), | |
| | | | | 4.42 (dd, 0.5H), 4.69 (dd, | |
| | | | | 0.5H), 4.77 (dd, 0.5H), | |
| | | | | 5.10-5.17 (m, 1H), | |
| | | | | 6.45 (dd, 1H), | |
| | | | | 6.67 (dd, 1H), 6.92 (bs, | |
| | | | | 1H), 7.38 (d, 1H), | |
| | | | | 7.49 (dd, 1H), 7.76 (d, 1H), | |
| | | | | 8.48 (s, 1H), 8.72 (s, | |
| | | | | 1H), 8.89 (s, 1H), | |
| | | | | 9.54 (bs, 1H) | |
| 27.5 | [3-[4-[(5-chloro-4- | 1-hydroxy | 507 | 0.76-0.87 (m, 2H), | 43 |
| | imidazo[1,2-a]pyridin-3- | cyclopropane | | 0.99-1.13 (m, 2H), | |
| | yl-pyrimidin-2- | carboxylic | | 3.77 (s, 3H), | |
| | yl)amino]-3-methoxy- | acid | | 3.80-3.89 (m, 1H), 4.32-4.44 (m, | |
| | phenoxy]azetidin-1-yl]- | | | 2H), 4.86-4.97 (m, | |
| | (1-hydroxy cyclopropyl)methanone | | | 1H), 5.07-5.15 (m, | |
| | | | | 1H), 6.12 (bs, 1H), | |
| | | | | 6.45 (dd, 1H), | |
| | | | | 6.69 (dd, 1H), 6.91 (bs, | |
| | | | | 1H), 7.38 (d, 1H), | |
| | | | | 7.48 (dd, 1H), 7.76 (d, 1H), | |
| | | | | 8.48 (s, 1H), 8.72 (s, | |
| | | | | 1H), 8.89 (s, 1H), | |
| | | | | 9.54 (bs, 1H) | |
| 27.6 | (2R)-1-[3-[4-[(5-chloro- | (S)-2- | 495 | 1.21 (d, 3H), 3.77 (s, | 36 |
| | 4-imidazo[1,2-a]pyridin- | hydroxy | | 3H), 3.81-3.87 (m, | |
| | 3-yl-pyrimidin-2- | propanoic | | 1H), 4.12-4.19 (m, | |
| | yl)amino]-3-methoxy- | acid | | 1H), 4.19-4.26 (m, | |
| | phenoxy]azetidin-1-yl]-2- | | | 1H), 4.32-4.41 (m, | |
| | hydroxy-propan-1-one | | | 1H), 4.70-4.80 (m, | |
| | | | | 1H), 5.08-5.15 (m, | |
| | | | | 1H), 5.17 (d, 0.5H), | |
| | | | | 5.19 (d, 0.5H), | |
| | | | | 6.44 (dd, 1H), 6.67 (dd, | |
| | | | | 1H), 6.92 (bs, 1H), | |
| | | | | 7.37 (d, 1H), 7.49 (dd, | |
| | | | | 1H), 7.76 (d, 1H), | |
| | | | | 8.48 (s, 1H), 8.72 (s, 1H), | |
| | | | | 8.89 (s, 1H), 9.54 (bs, | |
| | | | | 1H) | |

### Example 28

The following compounds were prepared using a similar procedure to that described in Example 22 using 5-chloro-4-imidazo[1,2-a]pyridin-3-yl-N-[2-methoxy-4-(4-piperidyl)phenyl]pyrimidin-2-amine (Example 4) and the appropriate acid as the starting materials and using DMF as a solvent. The acids used as the starting material are indicated in the following table.

| | | | | | % |
|---|---|---|---|---|---|
| Ex. No. | Name | Starting acid | MH+ | NMR spectrum | Yield |
| 28.1 | (2R)-1-[4-[4-[(5-chloro- | (R)-2- | 521 | 1.25 (d, 1.5H), 1.26 (d, | 81 |
| | 4-imidazo[1,2-a]pyridin- | methoxy | | 1.5H), 1.50-1.68 (m, | |
| | 3-yl-pyrimidin-2- | propanoic | | 2H), 1.83-1.94 (m 2H), | |
| | yl)amino]-3-methoxy- | acid | | 2.63-2.72 (m, 1H), | |
| | phenyl]-1-piperidyl]-2- | | | 2.79-2.89 (m, 1H), | |
| | methoxy-propan-1-one | | | 3.07-3.18 (m, 1H), | |
| | | | | 3.24 (s, 3H), 3.80 (s, 3H), | |
| | | | | 4.14-4.22 (m, 1H), | |
| | | | | 4.22-4.31 (m, 1H), | |
| | | | | 4.54-4.63 (m, 1H), | |
| | | | | 6.85 (d, 1H), 6.92 (dd, 1H), | |
| | | | | 7.02 (d, 1H), | |
| | | | | 7.46-7.54 (m, 2H), 7.76 (d, 1H), | |
| | | | | 8.50 (s, 1H), 8.73 (s, | |
| | | | | 1H), 8.89 (s, 1H), | |
| | | | | 9.60 (bs, 1H) | |
| 28.2 | (2R)-1-[4-[4-[(5-chloro- | (R)-2- | 507 | 1.21 (d, 1.5H), 1.24 (d, | 67 |
| | 4-imidazo[1,2-a]pyridin- | hydroxy | | 1.5H), 1.49-1.75 (m, | |
| | 3-yl-pyrimidin-2- | propanoic | | 2H), 1.81-1.92 (m, 2H), | |
| | yl)amino]-3-methoxy- | acid | | 2.63-2.74 (m, 1H), | |
| | phenyl]-1-piperidyl]-2- | | | 2.79-2.89 (m, 1H), | |
| | hydroxy-propan-1-one | | | 3.06-3.17 (m, 1H), | |
| | | | | 3.80 (s, 3H), 4.08-4.20 (m, | |
| | | | | 1H), 4.44-4.53 (m, 1H), | |
| | | | | 4.53-4.62 (m, 1H), | |
| | | | | 4.81 (d, 0.5H), 4.88 (d, | |
| | | | | 0.5H), 6.85 (d, 1H), | |
| | | | | 6.92 (dd, 1H), 7.02 (s, | |
| | | | | 1H), 7.46-7.55 (m, 2H), | |
| | | | | 7.77 (d, 1H), 8.50 (s, | |
| | | | | 1H), 8.73 (s, 1H), | |
| | | | | 8.89 (s, 1H), 9.60 (bs, 1H) | |
| 28.3 | (2S)-1-[4-[4-[(5-chloro- | (S)-2- | 521 | 1.25 (d, 1.5H), 1.26 (d, | 66 |
| | 4-imidazo[1,2-a]pyridin- | methoxy | | 1.5H), 1.50-1.68 (m, | |
| | 3-yl-pyrimidin-2- | propanoic | | 2H), 1.83-1.94 (m 2H), | |
| | yl)amino]-3-methoxy- | acid | | 2.63-2.72 (m, 1H), | |
| | phenyl]-1-piperidyl]-2- | | | 2.79-2.89 (m, 1H), | |
| | methoxy-propan-1-one | | | 3.07-3.18 (m, 1H), | |
| | | | | 3.24 (s, 3H), 3.80 (s, 3H), | |
| | | | | 4.14-4.22 (m, 1H), | |
| | | | | 4.22-4.31 (m, 1H), | |
| | | | | 4.54-4.63 (m, 1H), | |
| | | | | 6.85 (d, 1H), 6.92 (dd, 1H), | |
| | | | | 7.02 (d, 1H), | |
| | | | | 7.46-7.54 (m, 2H), 7.77 (d, 1H), | |
| | | | | 8.50 (s, 1H), 8.73 (s, | |
| | | | | 1H), 8.88 (s, 1H), | |
| | | | | 9.60 (bs, 1H) | |
| 28.4 | (2S)-1-[4-[4-[(5-chloro- | (S)-2- | 507 | 1.21 (d, 1.5H), 1.24 (d, | 81 |
| | 4-imidazo[1,2-a]pyridin- | hydroxy | | 1.5H), 1.50-1.73 (m, | |
| | 3-yl-pyrimidin-2- | propanoic | | 2H), 1.81-1.92 (m, 2H), | |
| | yl)amino]-3-methoxy- | acid | | 2.64-2.73 (m, 1H), | |
| | phenyl]-1-piperidyl]-2- | | | 2.79-2.89 (m, 1H), | |
| | hydroxy-propan-1-one | | | 3.06-3.18 (m, 1H), | |
| | | | | 3.80 (s, 3H), 4.08-4.20 (m, | |
| | | | | 1H), 4.44-4.53 (m, 1H), | |
| | | | | 4.53-4.62 (m, 1H), | |
| | | | | 4.81 (d, 0.5H), 4.88 (d, | |
| | | | | 0.5H), 6.85 (d, 1H), | |
| | | | | 6.92 (dd, 1H), 7.02 (s, | |
| | | | | 1H), 7.46-7.54 (m, 2H), | |
| | | | | 7.77 (d, 1H), 8.50 (s, | |
| | | | | 1H), 8.73 (s, 1H), | |
| | | | | 8.89 (s, 1H), 9.60 (bs, 1H) | |
| 28.5 | 1-[4-[4-[(5-chloro-4- | 2- | 493 | 1.52-1.63 (m, 1H), | |
| | imidazo[1,2-a]pyridin-3- | hydroxyacetic | | 1.63-1.75 (m, 1H), | |
| | yl-pyrimidin-2- | acid | | 1.80-1.90 (m, 2H), | |
| | yl)amino]-3-methoxy- | | | 2.67-2.77 (m, 1H), | |
| | phenyl]-1-piperidyl]-2- | | | 2.80-2.89 (m, 1H), | |
| | hydroxy-ethanone | | | 3.03-3.14 (m, 1H), | |
| | | | | 3.80 (s, 3H), 3.83, bs, 1H), | |
| | | | | 4.12 (dd, 1H), 4.18 (dd, | |
| | | | | 1H), 4.49 (t, 1H), | |
| | | | | 4.52-4.59 (m, 1H), 6.86 (dd, | |
| | | | | 1H), 6.93 (dd, 1H), | |
| | | | | 7.03 (d, 1H), 7.49 (d, 1H), | |
| | | | | 7.52 (ddd, 1H), 7.77 (d, | |
| | | | | 1H), 8.51 (s, 1H), | |
| | | | | 8.74 (s, 1H), 8.89 (s, 1H), | |
| | | | | 9.61 (bs, 1H) | |

### Example 29

### [4-[(5-chloro-4-imidazo[1,2-a]pyridin-3-yl-pyrimidin-2-yl)amino]-3-methoxy-phenyl]-[(1S,4S)-2,5-diazabicyclo[2.2.1]heptan-5-yl]methanone

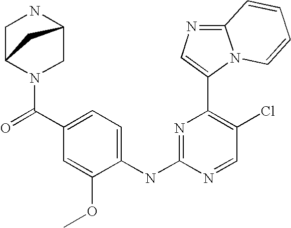

A mixture of 4-(5-chloro-4-(imidazo[1,2-a]pyridin-3-yl)pyrimidin-2-ylamino)-3-methoxybenzoic acid (see Example 29A, 100 mg, 0.25 mmol), (1S,4S)-tert-butyl 2,5-diazabicyclo[2.2.1]heptane-2-carboxylate, 2-(3H-[1,2,3]triazolo[4,5-b]pyridin-3-yl)-1,1,3,3-tetramethylisouronium hexafluorophosphate(V) (134 mg, 0.35 mmol) and N,N-diisopropylethylamine (0.132 ml, 0.76 mmol) in DMF (2 ml) was stirred at 25° C. for 4 hours. The reaction mixture was diluted with EtOAc and the organic phase was washed with water, brine then dried (MgSO₄), filtered and evaporated to dryness. The residue was taken up into dichloromethane (2 ml) and TFA (0.5 ml) was added. The solution was stirred one hour. After evaporation, the crude product was purified by preparative HPLC using a Waters X-Terra reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent to afford the title compound as a beige solid (43 mg, 36%); NMR spectrum: 1.54-1.60 (m, 0.6H), 1.62-1.68 (m, 4H), 1.70-1.75 (m, 0.6H), 1.78-1.85 (m, 0.4H), 2.85-2.93 (m, 1H), 2.95-3.00 (m, 0.6H), 3.00-3.06 (m, 0.4H), 3.23-3.28 (m, 1H), 3.47-3.53 (m, 0.6H), 3.60 (bs, 0.4H), 3.61-3.65 (m, 0.4H), 3.68 (bs, 0.6H), 3.87 (s, 3H), 4.29 (bs, 0.6H), 4.66 (bs, 0.4H), 6.99-7.08 (m, 1H), 7.08-7.12 (d, 0.6H), 7.17 (d, 0.4H), 7.20 (s, 0.6H), 7.25 (s, 0.4H), 7.50-7.57 (m, 1H), 7.79 (d, 1H), 7.83 (d, 1H), 8.60 (s, 1H), 8.75 (s, 1H), 9.00 (s, 0.4H), 9.04 (s, 0.6H), 9.65 (bs, 1H); Mass spectrum: 476 (MH+).

### Example 29A

### 4-(5-chloro-4-(imidazo[1,2-a]pyridin-3-yl)pyrimidin-2-ylamino)-3-methoxybenzoic acid

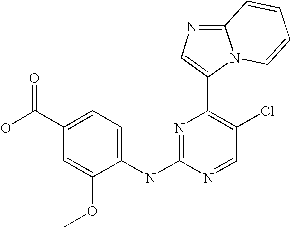

3-(2,5-dichloropyrimidin-4-yl)imidazo[1,2-a]pyridine (see Method 1, 530 mg, 2.00 mmol), methyl 4-amino-3-methoxybenzoate (described in Tetrahedron 2003, vol. 59, p. 5317, 400 mg, 2.2 mmol) and p-toluenesulfonic acid (761 mg, 4.0 mmol) were suspended in 2-pentanol (10 ml) and heated to 140° C. for 4 hours. After evaporation, the residue was diluted in dichloromethane and the mixture washed with a saturated aqueous solution of sodium bicarbonate. The solvent was removed and the crude product purified on silica gel eluting with 0 to 70% EtOAc in petroleum ether to afford methyl 4-(5-chloro-4-(imidazo[1,2-a]pyridin-3-yl)pyrimidin-2-ylamino)-3-methoxybenzoate (360 mg, 44%) as a pale yellow solid. This product was suspended in methanol (20 ml), treated with sodium hydroxide (3N in water, 1.2 ml, 3.7 mmol) and the resulting mixture was stirred at 80° C. for 2 hours. The reaction mixture was diluted with water, pH was adjusted to 5 with 6N HCl and the precipitate was filtered and dried (360 mg, 98%); NMR spectrum: 3.84 (s, 3H), 6.94 (dd, 1H), 7.51 (dd, 1H), 7.54 (d, 1H), 7.64 (s, 1H), 7.71 (d, 1H), 7.77 (d, 1H), 8.56 (s, 1H), 8.74 (s, 1H), 8.90 (s, 1H), 9.63 (bs, 1H).

### Example 30

### [4-[(5-chloro-4-imidazo[1,2-a]pyridin-3-yl-pyrimidin-2-yl)amino]-3-methoxy-phenyl]-piperazin-1-yl-methanone

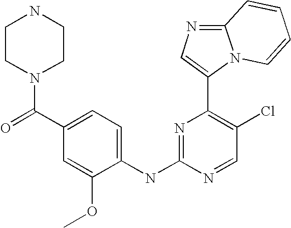

This compound was prepared following the procedure as described in Example 29 using 4-(5-chloro-4-(imidazo[1,2-a]pyridin-3-yl)pyrimidin-2-ylamino)-3-methoxybenzoic acid and tert-butyl piperazine-1-carboxylate as the starting materials (41%); NMR spectrum: 2.74 (bs, 4H), 3.47 (bs, 4H), 3.85 (s, 3H), 7.00 (dd, 1H), 7.03 (dd, 1H), 7.13 (d, 1H), 7.52 (ddd, 1H), 7.74-7.81 (m, 2H), 8.57 (s, 1H), 8.74 (s, 1H), 9.02 (s, 1H), 9.61 (bs, 1H); Mass spectrum: 464 (MH+).

### Example 31

### [4-[(5-chloro-4-imidazo[1,2-a]pyridin-3-yl-pyrimidin-2-yl)amino]-3-methoxy-phenyl]-[(1S,4S)-5-methyl-2,5-diazabicyclo[2.2.1]heptan-2-yl]methanone

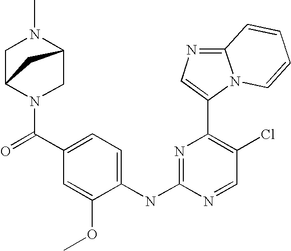

A mixture of 4-(5-chloro-4-(imidazo[1,2-a]pyridin-3-yl)pyrimidin-2-ylamino)-3-methoxybenzoic acid (see Example 29, 100 mg, 0.25 mmol), 2-methyl-2,5-diazabicyclo[2.2.1]heptane (56.7 mg, 0.51 mmol), 2-(3H-[1,2,3]triazolo[4,5-b]pyridin-3-yl)-1,1,3,3-tetramethylisouronium hexafluorophosphate(V) (134 mg, 0.35 mmol) and N,N-diisopropylethylamine (0.132 ml, 0.76 mmol) in DMF (2 ml) was stirred at 60° C. for 15 hours. The crude mixture was purified by preparative HPLC using a Waters X-Terra reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent to afford the title compound as a beige solid (53 mg, 42%); NMR spectrum: 1.65-1.71 (m, 0.6H), 1.75-1.81 (m, 1H), 1.82-1.88 (m, 0.4H), 2.28 (s, 1.2H), 2.36 (s, 1.8H), 2.65-2.69 (0.4H), 2.70-2.83 (m, 1.6H), 3.35-3.43 (m partially hidden by H2O, 1H), 3.43-3.53 (m, 2H), 3.85 (s, 1.8H), 3.86 (s, 1.2H), 4.22 (bs, 0.6H), 4.58 (bs, 0.4H), 7.06 (dd, 1H), 7.08 (d, 0.6H), 7.17 (d, 0.4H), 7.18 (s, 0.6H), 7.25 (s, 0.4H), 7.52 (ddd, 1H), 7.78 (d, 1H), 7.80-7.85 (m, 1H), 8.58 (s, 0.6H), 8.59 (s, 0.4H), 8.74 (s, 1H), 8.99 (s, 0.6H), 9.02 (s, 0.4H), 9.64 (bs, 1H); Mass spectrum: 490 (MH+).

### Example 32

### [4-[(5-chloro-4-imidazo[1,2-a]pyridin-3-yl-pyrimidin-2-yl)amino]-3-methoxy-phenyl]-[4-(2-hydroxyethyl)piperazin-1-yl]methanone

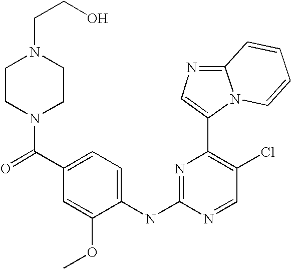

This compound was prepared following the procedure as described in Example 31 using 4-(5-chloro-4-(imidazo[1,2-a]pyridin-3-yl)pyrimidin-2-ylamino)-3-methoxybenzoic acid and 2-(piperazin-1-yl)ethanol as the starting materials (12%); NMR spectrum: 2.42 (t, 2H), 3.40 (bs, 4H), 3.48-3.56 (m, 2H), 3.59 (bs, 4H), 3.84 (s, 3H), 4.44 (t, 1H), 6.97-7.06 (m, 2H), 7.13 (s, 1H), 7.52 (ddd, 1H), 7.74-7.82 (m, 2H), 8.57 (s, 1H), 8.74 (s, 1H), 9.02 (s, 1H), 9.62 (bs, 1H); Mass spectrum: 508 (MH+).

### Example 33

### 4-[(5-chloro-4-imidazo[1,2-a]pyridin-3-yl-pyrimidin-2-yl)amino]-N-(2-hydroxyethyl)-3-methoxy-N-methyl-benzamide

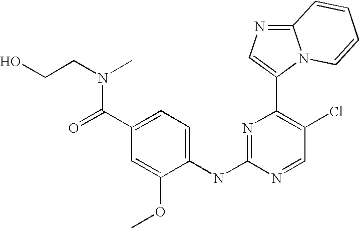

This compound was prepared following the procedure as described for Example 31, using 4-(5-chloro-4-(imidazo[1,2-a]pyridin-3-yl)pyrimidin-2-ylamino)-3-methoxybenzoic acid and 2-(methylamino)ethanol as the starting materials (40%); NMR spectrum: 3.01 (s, 3H), 3.43 (bs, 2H), 3.60 (bs, 2H), 3.83 (s, 3H), 4.70 (bs, 1H), 7.00-7.06 (m, 2H), 7.16 (s, 1H), 7.50 (ddd, 1H), 7.75 (d, 1H), 7.78 (d, 1H), 8.55 (s, 1H), 8.71 (s, 1H), 8.81 (s, 1H), 8.58 (s, 0.5H), 8.60 (s, 0.5H); Mass spectrum: 453 (MH+).

### Example 34

### 2-[4-[(5-chloro-4-imidazo[1,2-a]pyridin-3-yl-pyrimidin-2-yl)amino]-3-methoxy-phenyl]-1-piperazin-1-yl-ethanone

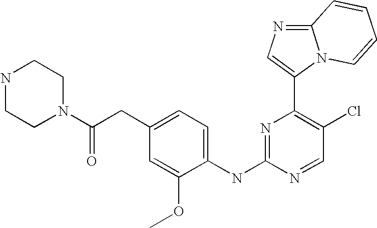

A mixture of 2-(4-(5-chloro-4-(imidazo[1,2-a]pyridin-3-yl)pyrimidin-2-ylamino)-3-methoxyphenyl)acetic acid (see Example 34A, 120 mg, 0.29 mmol), tert-butyl piperazine-1-carboxylate (60 mg, 0.32 mmol), 2-(3H-[1,2,3]triazolo[4,5-b]pyridin-3-yl)-1,1,3,3-tetramethylisouronium hexafluorophosphate(V) (156 mg, 0.41 mmol) and N,N-diisopropylethylamine (0.15 ml, 0.88 mmol) in DMF (1.5 ml) was stirred at 25° C. for 3 hours. The reaction mixture was diluted with EtOAc and the organic layer was washed with water, brine, dried (MgSO₄), filtered and evaporated to dryness. The residue was taken up into dichloromethane (1.5 ml) then TFA (0.5 ml) was added and the solution was stirred for one hour. After evaporation, the crude product was purified by preparative HPLC using a Waters X-Terra reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent to afford 2-(4-(5-chloro-4-(imidazo[1,2-a]pyridin-3-yl)pyrimidin-2-ylamino)-3-methoxyphenyl)-1-(piperazin-1-yl)ethanone (58 mg, 41%) as a yellow solid; NMR spectrum: 2.54-2.59 (m, 2H), 2.59-2.63 (m, 2H), 3.39-3.45 (m, 4H), 3.74 (s, 2H), 3.76 (s, 3H), 6.84 (d, 1H), 6.97 (dd, 1H), 7.01 (s, 1H), 7.46-7.53 (m, 2H), 7.76 (d, 1H), 8.51 (s, 1H), 8.74 (s, 1H), 8.91 (s, 1H), 9.58 (bs, 1H); Mass spectrum: 478 (MH+).

### Example 34A

### 2-(4-(5-chloro-4-(imidazo[1,2-a]pyridin-3-yl)pyrimidin-2-ylamino)-3-methoxyphenyl)acetic acid

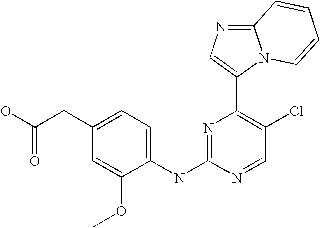

A mixture of 3-(2,5-dichloropyrimidin-4-yl)imidazo[1,2-a]pyridine (see Method 1, 820 mg, 3.09 mmol), 2-(4-amino-3-methoxyphenyl)acetic acid (see Method 37, 560 mg, 3.09 mmol) and 4-methylbenzenesulfonic acid hydrate (1.17 g, 6.19 mmol) in DMA (10 ml) was stirred at 130° C. for 3 hours. The reaction mixture was partitioned between water and dichloromethane and the organic phase was evaporated. The crude product was purified by flash chromatography on silica gel eluting with 2 to 10% MeOH/CH2Cl2 to afford 2-(4-(5-chloro-4-(imidazo[1,2-a]pyridin-3-yl)pyrimidin-2-ylamino)-3-methoxyphenyl)acetic acid (336 mg, 26%); NMR spectrum: 3.61 (s, 2H), 3.76 (s, 3H), 6.88 (dd, 1H), 6.97 (dd, 1H), 7.05 (s, 1H), 7.44-7.53 (m, 2H), 7.76 (d, 1H), 8.51 (s, 1H), 8.75 (s, 1H), 8.93 (s, 1H), 9.58 (bs, 1H), 12.46 (bs, 1H).

### Example 35

### 2-[4-[(5-chloro-4-imidazo[1,2-a]pyridin-3-yl-pyrimidin-2-yl)amino]-3-methoxy-phenyl]-N-pyrrolidin-3-yl-acetamide

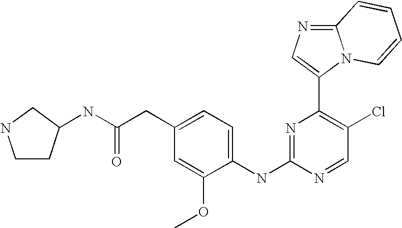

This compound was prepared following the procedure as described for Example 34, using 2-(4-(5-chloro-4-(imidazo[1,2-a]pyridin-3-yl)pyrimidin-2-ylamino)-3-methoxyphenyl)acetic acid and tert-butyl 3-aminopyrrolidine-1-carboxylate as the starting materials (33%); NMR spectrum: 1.43-1.55 (m, 0.7H), 1.70-1.80 (m, 0.3H), 1.82-1.95 (m, 0.7H), 1.96-2.07 (m, 0.3H), 2.70-2.77 (m, 0.7H), 2.80-2.88 (m, 0.7H), 2.88-2.96 (m, 0.7H), 3.07-3.14 (m, 0.3H), 2.34-2.40 m partially hidden by H2O, 1.6H), 3.41 (s, 1.4H), 3.45 (s, 0.6H), 3.76 (s, 3H), 4.05-4.13 (0.7H), 4.18-4.26 (m, 0.3H), 6.86 (dd, 1H), 6.94 (dd, 1H), 7.05 (s, 1H), 7.42-7.52 (m, 2H), 7.76 (d, 1H), 8.19 (d, 0.7H), 8.39 (d, 0.3H), 8.50 (s, 1H), 8.75 (s, 1H), 8.92 (s, 1H), 9.54 (bs, 1H); Mass spectrum: 478 (MH+).

### Example 36

### 2-[4-[(5-chloro-4-imidazo[1,2-a]pyridin-3-yl-pyrimidin-2-yl)amino]-3-methoxy-phenyl]-1-morpholino-ethanone

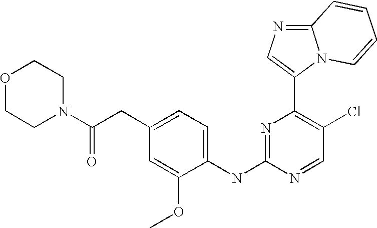

A mixture of 2-(4-(5-chloro-4-(imidazo[1,2-a]pyridin-3-yl)pyrimidin-2-ylamino)-3-methoxyphenyl)acetic acid (see Example 34, 107 mg, 0.26 mmol), morpholine (0.025 ml, 0.28 mmol), 2-(3H-[1,2,3]triazolo[4,5-b]pyridin-3-yl)-1,1,3,3-tetramethylisouronium hexafluorophosphate(V) (137 mg, 0.36 mmol) N,N-diisopropylethylamine (0.13 ml, 0.77 mmol) in DMA (1.5 ml) was stirred at 25° C. for 3 hours. The reaction mixture was purified by preparative HPLC using a Waters X-Terra reverse-phase column (5 microns silica, 30 mm diameter, 150 mm length) and decreasingly polar mixtures of water (containing 0.2% ammonium carbonate) and acetonitrile as eluent to afford 2-(4-(5-chloro-4-(imidazo[1,2-a]pyridin-3-yl)pyrimidin-2-ylamino)-3-methoxyphenyl)-1-morpholinoethanone (18 mg, 14%) as a yellow solid; NMR spectrum: 3.46-3.58 (m, 8H), 3.76 (s, 2H), 3.77 (s, 3H), 6.84 (dd, 1H), 6.98 (dd, 1H), 7.01 (d, 1H), 7.47-7.53 (m, 2H), 7.77 (d, 1H), 8.51 (s, 1H), 8.74 (s, 1H), 8.92 (s, 1H), 9.59 (bs, 1H); Mass spectrum: 479 (MH+).

### Example 37

### 2-[4-[(5-chloro-4-imidazo[1,2-a]pyridin-3-yl-pyrimidin-2-yl)amino]-3-methoxy-phenyl]-1-[4-(2-hydroxyethyl)piperazin-1-yl]ethanone

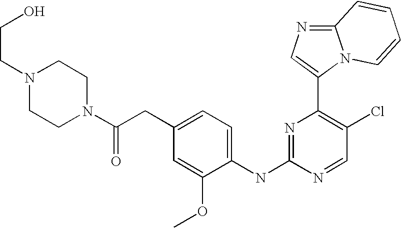

This compound was prepared following the procedure as described for Example 36, using 2-(4-(5-chloro-4-(imidazo[1,2-a]pyridin-3-yl)pyrimidin-2-ylamino)-3-methoxyphenyl)acetic acid and 2-(piperazin-1-yl)ethanol as the starting materials (55%); NMR spectrum: 2.31-2.40 (m, 6H), 3.46-3.55 (m, 6H), 3.75 (s, 2H), 3.77 (s, 3H), 4.42 (t, 1H), 6.85 (dd, 1H), 6.98 (dd, 1H), 7.02 (d, 1H), 7.48-7.54 (m, 2H), 7.77 (ddd, 1H), 8.52 (s, 1H), 8.75 (s, 1H), 8.92 (s, 1H), 9.59 (bs, 1H); Mass spectrum: 522 (MH+).

### Example 38

### 2-[4-[(5-chloro-4-imidazo[1,2-a]pyridin-3-yl-pyrimidin-2-yl)amino]-3-methoxy-phenyl]-1-[(3S)-3-hydroxypyrrolidin-1-yl]ethanone

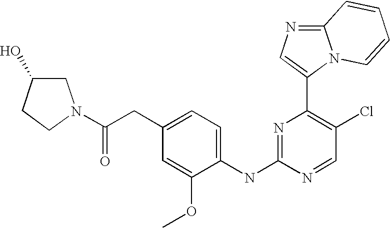

This compound was prepared following the procedure as described for Example 36, using 2-(4-(5-chloro-4-(imidazo[1,2-a]pyridin-3-yl)pyrimidin-2-ylamino)-3-methoxyphenyl)acetic acid and (S)-pyrrolidin-3-ol as the starting materials (33%); NMR spectrum: 1.69-2.02 (m, 2H), 3.32-3.50 (m partially hidden by H₂O, 3H), 3.55-3.71 (m, 3H), 3.76 (s, 3H), 4.26 (m, 0.5H), 4.32 (m, 0.5H), 4.93 (d, 0.5H), 5.03 (d, 0.5H), 6.86 (d, 1H), 6.98 (dd, 1H), 7.02 (s, 1H), 7.43-7.55 (m, 2H), 7.76 (d, 1H), 8.51 (s, 1H), 8.74 (s, 1H), 8.81 (s, 1H), 9.58 (bs, 1H); Mass spectrum: 479 (MH+).

### Example 39

### 1-[4-[4-[(5-chloro-4-imidazo[1,2-a]pyridin-3-yl-pyrimidin-2-yl)amino]-3-methoxy-phenyl]piperazin-1-yl]-2-methoxy-ethanone

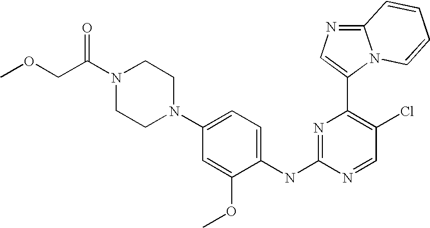

2-Methoxyacetyl chloride (0.021 mL, 0.23 mmol) was added to a stirred solution of 5-chloro-4-(imidazo[1,2-a]pyridin-3-yl)-N-(2-methoxy-4-(piperazin-1-yl)phenyl)pyrimidin-2-amine (Example 2, 100 mg, 0.23 mmol) and N,N-diisopropylethylamine (0.052 mL, 0.30 mmol) in dichloromethane (2 mL) at 25° C. under nitrogen and the mixture was stirred for 1 hour. The volatiles were evaporated and the residue was purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent to afford 1-(4-(4-(5-chloro-4-(imidazo[1,2-a]pyridin-3-yl)pyrimidin-2-ylamino)-3-methoxyphenyl)piperazin-1-yl)-2-methoxyethanone (73 mg, 62%) as a pale yellow solid; NMR spectrum: 3.15-3.24 (m, 4H), 3.32 (s, 3H), 3.54-3.60 (m, 2H), 3.60-3.66 (m, 2H), 3.77 (s, 3H), 4.15 (s, 2H), 6.56 (dd, 1H), 6.74 (d, 1H), 6.89 (bs, 1H), 7.30 (d, 1H), 7.49 (ddd, 1H), 7.75 (d, 1H), 8.46 (s, 1H), 8.72 (s, 1H), 8.80 (s, 1H), 9.55 (bs, 1H); Mass spectrum: 508 (MH+).

### Example 40

### 1-[4-[4-[(5-chloro-4-imidazo[1,2-a]pyridin-3-yl-pyrimidin-2-yl)amino]-3-methoxy-phenyl]piperazin-1-yl]-2-hydroxy-ethanone

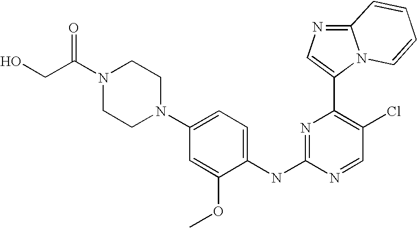

2-Chloro-2-oxoethyl acetate (0.030 mL, 0.28 mmol) was added to a stirred solution of 5-chloro-4-(imidazo[1,2-a]pyridin-3-yl)-N-(2-methoxy-4-(piperazin-1-yl)phenyl)pyrimidin-2-amine (Example 2, 120 mg, 0.28 mmol) and N,N-diisopropylethylamine (0.062 mL, 0.36 mmol) in CH₂Cl₂ (2 mL) at 25° C. under nitrogen. The mixture was stirred at 25° C. for 1 hour then MeOH (2 ml) and K2CO3 (10 eq.) were added and the reaction mixture was stirred at 50° C. for 1 hour. The volatiles were evaporated and the residue was purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent to afford 1-(4-(4-(5-chloro-4-(imidazo[1,2-a]pyridin-3-yl)pyrimidin-2-ylamino)-3-methoxyphenyl)piperazin-1-yl)-2-hydroxyethanone (83 mg, 61%) as a yellow solid; NMR spectrum: 3.20 (bs, 4H), 3.53 (bs, 2H), 3.65 (bs, 2H), 3.77 (s, 3H), 4.16 (d, 2H), 4.65 (t, 1H), 6.56 (dd, 1H), 6.74 (d, 1H), 6.89 (bs, 1H), 7.30 (d, 1H), 7.49 (ddd, 1H), 7.75 (d, 1H), 8.46 (s, 1H), 8.72 (s, 1H), 8.81 (s, 1H), 9.55 (bs, 1H); Mass spectrum: 494 (MH+).

### Example 41

### (2S)-1-[4-[4-[(5-chloro-4-imidazo[1,2-a]pyridin-3-yl-pyrimidin-2-yl)amino]-3-methoxy-phenyl]piperazin-1-yl]-2-hydroxy-propan-1-one

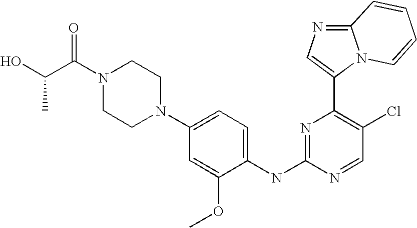

This compound was prepared following the procedure as described in Example 40, using 5-chloro-4-(imidazo[1,2-a]pyridin-3-yl)-N-(2-methoxy-4-(piperazin-1-yl)phenyl)pyrimidin-2-amine and (S)-1-chloro-1-oxopropan-2-yl acetate (66%); NMR spectrum: 1.23 (d, 3H), 3.17 (bs, 1H), 3.21 (bs, 3H), 3.62 (bs, 1H), 3.70 (bs, 3H), 3.77 (s, 3H), 4.46-4.52 (m, 1H), 4.99 (d, 1H), 6.57 (dd, 1H), 6.75 (d, 1H), 6.90 (bs, 1H), 7.30 (d, 1H), 7.49 (dd, 1H), 7.75 (d, 1H), 8.46 (s, 1H), 8.72 (s, 1H), 8.81 (s, 1H), 9.55 (bs, 1H); Mass spectrum: 508 (MH+).

### Example 42

### 1-[4-[4-[(5-chloro-4-imidazo[1,2-a]pyridin-3-yl-pyrimidin-2-yl)amino]-3-methoxy-phenyl]piperazin-1-yl]propan-1-one

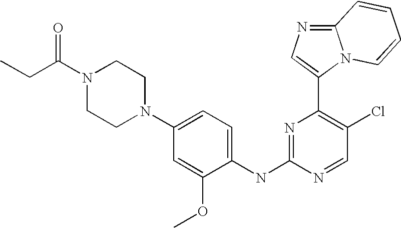

This compound was prepared following the procedure as described in Example 39, using 5-chloro-4-(imidazo[1,2-a]pyridin-3-yl)-N-(2-methoxy-4-(piperazin-1-yl)phenyl)pyrimidin-2-amine and propionyl chloride as the starting materials (74%); NMR spectrum: 1.02 (t, 3H), 2.39 (q, 1H), 3.13-3.18 (m, 2H), 3.18-3.23 (m, 2H), 3.59-3.67 (m, 4H), 3.76 (s, 3H), 6.56 (dd, 1H), 6.74 (d, 1H), 6.89 (bs, 1H), 7.30 (d, 1H), 7.49 (ddd, 1H), 7.75 (d, 1H), 8.45 (s, 1H), 8.72 (s, 1H), 8.80 (s, 1H), 9.56 (bs, 1H); Mass spectrum: 492 (MH+).

### Example 43

### [4-[4-[(5-chloro-4-imidazo[1,2-a]pyridin-3-ylpyrimidin-2-yl)amino]-3-methoxyphenyl]piperazin-1-yl]-cyclopropylmethanone

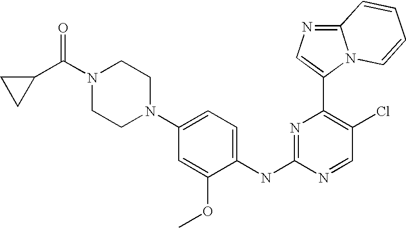

This compound was prepared following the procedure as described in Example 39, using 5-chloro-4-(imidazo[1,2-a]pyridin-3-yl)-N-(2-methoxy-4-(piperazin-1-yl)phenyl)pyrimidin-2-amine and cyclopropanecarbonyl chloride as the starting materials (75%); NMR spectrum: 0.71-0.80 (m, 4H), 2.02-2.09 (m, 1H), 3.17 (bs, 2H), 3.24 (bs, 2H), 3.65 (bs, 2H), 3.77 (s, 3H), 3.86 (bs, 2H), 6.57 (dd, 1H), 6.75 (d, 1H), 6.90 (bs, 1H), 7.30 (d, 1H), 7.50 (ddd, 1H), 7.75 (d, 1H), 8.46 (s, 1H), 8.72 (s, 1H), 8.80 (s, 1H), 9.56 (bs, 1H); Mass spectrum: 504 (MH+).

### Example 44

### methyl 4-[4-[(5-chloro-4-imidazo[1,2-a]pyridin-3-yl-pyrimidin-2-yl)amino]-3-methoxy-phenyl]piperazine-1-carboxylate

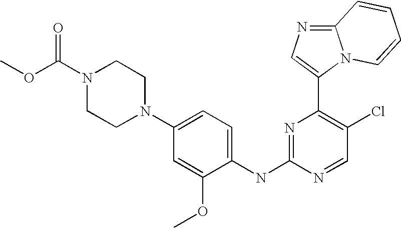

Methyl chloroformate (0.018 mL, 0.23 mmol) was added to a stirred solution of 5-chloro-4-(imidazo[1,2-a]pyridin-3-yl)-N-(2-methoxy-4-(piperazin-1-yl)phenyl)pyrimidin-2-amine (Example 2, 100 mg, 0.23 mmol) and N,N-diisopropylethylamine (0.052 mL, 0.30 mmol) in dichloromethane (2 mL) at 25° C. under nitrogen. The mixture was stirred at 25° C. for 1 hour then the solvent was evaporated and the residue was purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent to afford methyl 4-(4-(5-chloro-4-(imidazo[1,2-a]pyridin-3-yl)pyrimidin-2-ylamino)-3-methoxyphenyl)piperazine-1-carboxylate (39 mg, 34%) as a pale yellow solid; NMR spectrum: 3.15-3.21 (m, 4H), 3.51-3.58 (m, 4H), 3.64 (s, 3H), 3.76 (s, 3H), 6.56 (dd, 1H), 6.74 (d, 1H), 6.89 (bs, 1H), 7.30 (d, 1H), 7.50 (ddd, 1H), 7.75 (d, 1H), 8.46 (s, 1H), 8.72 (s, 1H), 8.80 (s, 1H), 9.55 (bs, 1H); Mass spectrum: 494 (MH+).

### Example 45

### 2-methoxyethyl 4-[4-[(5-chloro-4-imidazo[1,2-a]pyridin-3-yl-pyrimidin-2-yl)amino]-3-methoxy-phenyl]piperazine-1-carboxylate

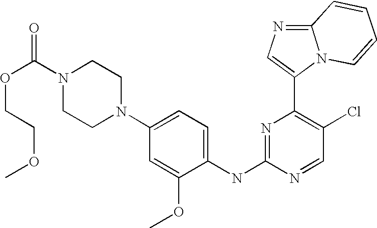

This compound was prepared following the procedure as described in Example 44, using 5-chloro-4-(imidazo[1,2-a]pyridin-3-yl)-N-(2-methoxy-4-(piperazin-1-yl)phenyl)pyrimidin-2-amine and 2-methoxyethyl chloroformate as the starting materials (39%); NMR spectrum: 3.14-3.22 (m, 4H), 3.29 (s, 3H), 3.51-3.61 (m, 6H), 3.76 (s, 3H), 4.13-4.19 (m, 2H), 6.56 (dd, 1H), 6.74 (d, 1H), 6.89 (bs, 1H), 7.30 (d, 1H), 7.50 (ddd, 1H), 7.75 (d, 1H), 8.46 (s, 1H), 8.72 (s, 1H), 8.81 (s, 1H), 9.55 (bs, 1H); Mass spectrum: 538 (MH+).

### Example 46

### [4-[4-[(5-chloro-4-imidazo[1,2-a]pyridin-3-yl-pyrimidin-2-yl)amino]-3-methoxy-phenyl]piperazin-1-yl]-morpholino-methanone

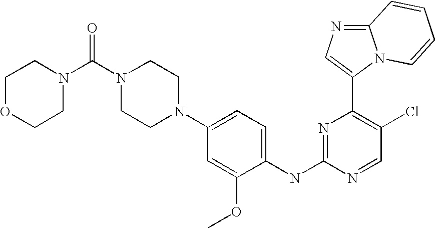

4-Nitrophenyl chloroformate (32 mg, 0.16 mmol) in dichloromethane (5 mL) was added to a stirred solution of 5-chloro-4-(imidazo[1,2-a]pyridin-3-yl)-N-(2-methoxy-4-(piperazin-1-yl)phenyl)pyrimidin-2-amine (Example 2, 70 mg, 0.16 mmol) and N,N-diisopropylethylamine (0.056 mL, 0.32 mmol) dissolved in dichloromethane (5 mL) at 25° C. under nitrogen. After 15 minutes, DMF (2 mL) and morpholine (1 mL) were added and the mixture was heated at 110° C. for 10 minutes. The reaction mixture was partitioned between water and dichloromethane and the organic phase was separated, dried and evaporated. The residue was purified by preparative HPLC using a Waters X-Bridge reverse-phase column (5 microns silica, 19 mm diameter, 100 mm length) and decreasingly polar mixtures of water (containing 0.2% ammonium carbonate) and acetonitrile as eluent to afford (4-(4-(5-chloro-4-(imidazo[1,2-a]pyridin-3-yl)pyrimidin-2-ylamino)-3-methoxyphenyl)piperazin-1-yl)(morpholino)methanone (41 mg, 46%) as a pale yellow solid; NMR spectrum: 3.15-3.24 (m, 8H), 3.33-3.38 (m partially hidden by H₂O, 4H), 3.56-3.63 (m, 4H), 3.76 (s, 3H), 6.56 (dd, 1H), 6.73 (d, 1H), 2.89 (bs, 1H), 7.30 (d, 1H), 7.49 (dd, 1H), 7.75 (d, 1H), 8.45 (s, 1H), 8.72 (s, 1H), 8.79 (s, 1H), 9.56 (bs, 1H); Mass spectrum: 549 (MH+).

### Example 47

The following compounds were prepared using a similar procedure to that described in Example 46 using 5-chloro-4-(imidazo[1,2-a]pyridin-3-yl)-N-(2-methoxy-4-(piperazin-1-yl)phenyl)pyrimidin-2-amine and an appropriate amine as the starting materials. The amines used as the starting material are indicated in the following table.

| | | Starting | | | % |
|---|---|---|---|---|---|
| Ex. No. | Name | amine | MH+ | NMR spectrum | Yield |
| 47.1 | 4-[4-[(5-chloro-4- | 2- | 537 | 2.87 (s, 3H), | 38 |
| | imidazo[1,2-a]pyridin-3-yl- | (methylamino)ethanol | | 3.17-3.23 (m, 4H), 3.24 (t, 2H), | |
| | pyrimidin-2-yl)amino]-3- | | | 3.26-3.32 (m, 4H), | |
| | methoxy-phenyl]-N-(2- | | | 3.53-3.59 (m, 2H), | |
| | hydroxyethyl)-N-methyl- | | | 3.76 (s, 3H), 4.72 (bs, | |
| | piperazine-1-carboxamide | | | 1H), 6.56 (dd, 1H), | |
| | | | | 6.73 (d, 1H), 6.89 (bs, | |
| | | | | 1H), 7.30 (d, 1H), | |
| | | | | 7.49 (ddd, 1H), 7.75 (d, | |
| | | | | 1H), 8.46 (s, 1H), | |
| | | | | 8.72 (s, 1H), 8.79 (s, 1H), | |
| | | | | 9.55 (bs, 1H) | |
| 47.2 | 4-[4-[(5-chloro-4- | dimethylamine | 507 | 2.80 (s, 6H), | 46 |
| | imidazo[1,2-a]pyridin-3-yl- | | | 3.16-3.23 (m, 4H), 3.24-3.32 (m, | |
| | pyrimidin-2-yl)amino]-3- | | | 4H), 3.77 (s, 3H), | |
| | methoxy-phenyl]-N,N- | | | 6.56 (dd, 1H), 6.73 (d, 1H), | |
| | dimethyl-piperazine-1- | | | 6.89 (bs, 1H), 7.30 (d, | |
| | carboxamide | | | 1H), 7.49 (ddd, 1H), | |
| | | | | 7.75 (d, 1H), 8.46 (s, | |
| | | | | 1H), 8.72 (s, 1H), | |
| | | | | 8.79 (s, 1H), 9.56 (bs, 1H) | |
| 47.3 | [4-[4-[(5-chloro-4- | piperazine | 548 | 2.29 (bs, 1H), | 77 |
| | imidazo[1,2-a]pyridin-3-yl- | | | 2.46 (bs, 2H), 2.67-2.72 m, | |
| | pyrimidin-2-yl)amino]-3- | | | 2H), 3.10-3.15 (m, | |
| | methoxy-phenyl]piperazin- | | | 2H), 3.15-3.25 (m, | |
| | 1-yl]-piperazin-1-yl- | | | 6H), 3.27-3.32 (m | |
| | methanone | | | partially hidden by | |
| | | | | H2O, 4H), 3.76 (s, | |
| | | | | 3H), 6.55 (dd, 1H), | |
| | | | | 6.73 (d, 1H), 6.90 (bs, | |
| | | | | 1H), 7.30 (d, 1H), | |
| | | | | 7.49 (ddd, 1H), 7.75 (d, | |
| | | | | 1H), 8.46 (s, 1H), | |
| | | | | 8.72 (s, 1H), 8.79 (s, 1H), | |
| | | | | 9.56 (bs, 1H) | |

### Example 48

### 5-chloro-4-imidazo[1,2-a]pyridin-3-yl-N-[2-methoxy-4-[4-(2-methylsulfonylethyl)piperazin-1-yl]phenyl]pyrimidin-2-amine

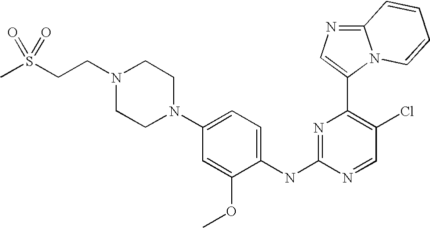

A mixture of 5-chloro-4-(imidazo[1,2-a]pyridin-3-yl)-N-(2-methoxy-4-(piperazin-1-yl)phenyl)pyrimidin-2-amine (Example 2, 80 mg, 0.18 mmol), ammonium cerium (IV) nitrate (10 mg, 0.02 mmol) and methylsulfonylethene (0.80 mL, 9.1 mmol) in water (1 mL)/THF (1 mL) was stirred at 25° C. for 1 day. The reaction mixture was extracted with dichloromethane and the organic phase was dried over magnesium sulfate and concentrated to afford the crude product which was purified by preparative HPLC using a Waters X-Terra reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent to provide 5-chloro-4-(imidazo[1,2-a]pyridin-3-yl)-N-(2-methoxy-4-(4-(2-(methylsulfonyl)ethyl)piperazin-1-yl)phenyl)pyrimidin-2-amine (55 mg, 55%) as a pale yellow solid; NMR spectrum: 2.59-2.66 (m, 4H), 2.79 (t, 2H), 3.06 (s, 3H), 3.17-3.23 (m, 4H), 3.36 (t, 2H), 3.76 (s, 3H), 6.54 (dd, 1H), 6.71 (d, 1H), 6.88 (bs, 1H), 7.27 (d, 1H), 7.49 (ddd, 1H), 7.75 (d, 1H), 8.45 (s, 1H), 8.72 (s, 1H), 8.78 (s, 1H), 9.55 (bs, 1H); Mass spectrum: 542 (MH+).

### Example 49

### 3-[4-[4-[(5-chloro-4-imidazo[1,2-a]pyridin-3-yl-pyrimidin-2-yl)amino]-3-methoxy-phenyl]piperazin-1-yl]propanenitrile

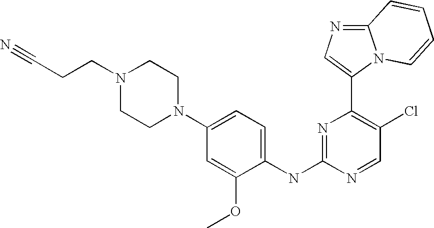

This compound was prepared following the procedure as described in Example 48 using 5-chloro-4-(imidazo[1,2-a]pyridin-3-yl)-N-(2-methoxy-4-(piperazin-1-yl)phenyl)pyrimidin-2-amine and acrylonitrile as the starting materials (49%); NMR spectrum: 2.59-2.64 (m, 4H), 2.65 (t, 2H), 2.74 (t, 2H), 3.17-3.24 (m, 4H), 3.76 (s, 3H), 6.54 (dd, 1H), 6.71 (d, 1H), 6.88 (bs, 1H), 7.27 (d, 1H), 7.50 (ddd, 1H), 7.75 (d, 1H), 8.45 (s, 1H), 8.72 (s, 1H), 8.79 (s, 1H), 9.55 (bs, 1H); Mass spectrum: 489 (MH+).

### Example 50

### (2S)-1-[4-[4-[(5-chloro-4-imidazo[1,2-a]pyridin-3-yl-pyrimidin-2-yl)amino]-3-methoxy-phenyl]piperazin-1-yl]propan-2-ol

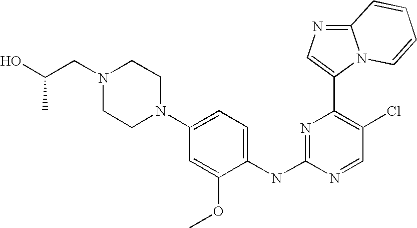

A mixture of 5-chloro-4-(imidazo[1,2-a]pyridin-3-yl)-N-(2-methoxy-4-(piperazin-1-yl)phenyl)pyrimidin-2-amine (Example 2, 80 mg, 0.18 mmol) and (S)-2-methyloxirane (0.064 mL, 0.92 mmol) in ethanol (1.5 mL) was placed into a microwave tube and heated at 140° C. for 4 minutes in a microwave reactor. The crude product was purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford (2S)-1-(4-(4-(5-chloro-4-(imidazo[1,2-a]pyridin-3-yl)pyrimidin-2-ylamino)-3-methoxyphenyl)piperazin-1-yl)propan-2-ol (53 mg, 58%) as a pale yellow solid; NMR spectrum: 1.08 (d, 3H), 2.24 (dd, 1H), 2.32 (dd, 1H), 2.55-2.63 (m, 4H), 3.15-3.23 (m, 4H), 3.75 (s, 3H), 3.78-3.87 (m, 1H), 4.33 (d, 1H), 6.53 (dd, 1H), 6.69 (d, 1H), 6.88 (bs, 1H), 7.26 (d, 1H), 7.49 (dd, 1H), 7.75 (d, 1H), 8.45 (s, 1H), 8.72 (s, 1H), 8.78 (s, 1H), 9.55 (bs, 1H); Mass spectrum: 494 (MH+).

### Example 51

### (2R)-1-[4-[4-[(5-chloro-4-imidazo[1,2-a]pyridin-3-yl-pyrimidin-2-yl)amino]-3-methoxy-phenyl]piperazin-1-yl]propan-2-ol

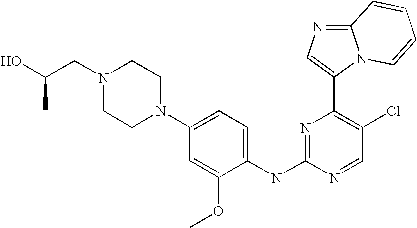

This compound was prepared following the procedure as described in Example 50 using 5-chloro-4-(imidazo[1,2-a]pyridin-3-yl)-N-(2-methoxy-4-(piperazin-1-yl)phenyl)pyrimidin-2-amine and (R)-2-methyloxirane as the starting materials (56%); NMR spectrum: 1.08 (d, 3H), 2.24 (dd, 1H), 2.32 (dd, 1H), 2.56-2.63 (m, 4H), 3.15-3.22 (m, 4H), 3.75 (s, 3H), 3.78-3.87 (ms, 1H), 4.34 (d, 1H), 6.53 (dd, 1H), 6.69 (d, 1H), 6.88 (m, 1H), 7.26 (d, 1H), 7.45 (ddd, 1H), 7.75 (d, 1H), 8.45 (s, 1H), 8.72 (s, 1H), 8.78 (s, 1H), 9.54 (bs, 1H); Mass spectrum: 494 (MH+).

### Example 52

### 1-[4-[4-[(5-chloro-4-imidazo[1,2-a]pyridin-3-yl-pyrimidin-2-yl)amino]-3-methoxy-phenyl]piperazin-1-yl]-2-methyl-propan-2-ol

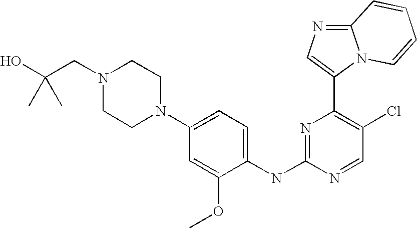

This compound was prepared following the procedure as described in Example 50 using 5-chloro-4-(imidazo[1,2-a]pyridin-3-yl)-N-(2-methoxy-4-(piperazin-1-yl)phenyl)pyrimidin-2-amine and 2,2-dimethyloxirane as the starting materials (68%); NMR spectrum: 1.13 (s, 6H), 2.27 (s, 2H), 2.65-2.75 (m, 4H), 3.14-3.23 (m, 4H), 3.75 (s, 3H), 4.14 (s, 1H), 6.52 (dd, 1H), 6.68 (d, 1H), 6.89 (bs, 1H), 7.26 (d, 1H), 7.50 (ddd, 1H), 7.75 (d, 1H), 8.45 (s, 1H), 8.72 (s, 1H), 8.78 (s, 1H), 9.56 (bs, 1H); Mass spectrum: 508 (MH+).

### Example 53

### (2R)-3-[4-[4-[(5-chloro-4-imidazo[1,2-a]pyridin-3-yl-pyrimidin-2-yl)amino]-3-methoxy-phenyl]piperazin-1-yl]propane-1,2-diol

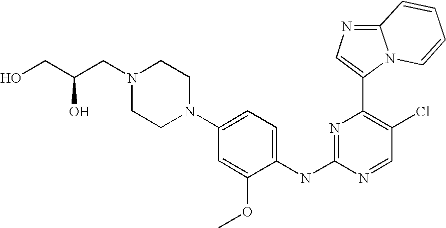

This compound was prepared following the procedure as described in Example 50 using 5-chloro-4-(imidazo[1,2-a]pyridin-3-yl)-N-(2-methoxy-4-(piperazin-1-yl)phenyl)pyrimidin-2-amine and (S)-oxiran-2-ylmethanol as the starting materials (73%); NMR spectrum: 2.31 (dd, 1H), 2.45 (dd, 1H), 2.55-2.68 (m, 4H), 3.14-3.23 (m, 4H), 3.33-3.39 (m, 2H), 3.63-3.70 (m, 1H), 3.75 (s, 3H), 4.45 (d, 1H), 4.55 (t, 1H), 6.53 (dd, 1H), 6.60 (d, 1H), 6.88 (bs, 1H), 7.26 (d, 1H), 7.50 (ddd, 1H), 7.75 (d, 1H), 8.45 (s, 1H), 8.72 (s, 1H), 8.78 (s, 1H), 9.54 (bs, 1H); Mass spectrum: 510 (MH+).

### Example 54

### 4-[4-[(5-chloro-4-imidazo[1,2-a]pyridin-3-yl-pyrimidin-2-yl)amino]-3-methoxy-phenyl]piperazine-1-carboxamide

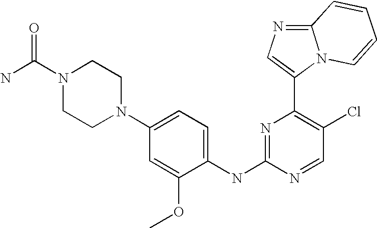

Trimethylsilyl isocyanate (0.201 mL, 1.49 mmol) was added to a stirred solution of 5-chloro-4-(imidazo[1,2-a]pyridin-3-yl)-N-(2-methoxy-4-(piperazin-1-yl)phenyl)pyrimidin-2-amine (Example 2, 108 mg, 0.25 mmol) in dichloromethane (3 mL) and the mixture was stirred at 25° C. for 1 hour. After evaporation, the crude product was purified by flash chromatography on silica gel eluting with 0 to 10% MeOH/ethyl acetate to afford 4-(4-(5-chloro-4-(imidazo[1,2-a]pyridin-3-yl)pyrimidin-2-ylamino)-3-methoxyphenyl)piperazine-1-carboxamide (100 mg, 84%) as a pale yellow solid; NMR spectrum: 3.10-3.20 (m, 4H), 3.43-3.52 (m, 4H), 3.77 (s, 3H), 6.08 (s, 2H), 6.56 (dd, 1H), 6.74 (d, 1H), 6.89 (bs, 1H), 7.29 (d, 1H), 7.50 (ddd, 1H), 7.75 (s, 1H), 8.45 (s, 1H), 8.72 (s, 1H), 8.80 (s, 1H), 9.55 (bs, 1H); Mass spectrum: 479 (MH+).

### Example 55

### 4-[3-[(5-chloro-4-imidazo[1,2-a]pyridin-3-yl-pyrimidin-2-yl)amino]-4-methoxy-phenyl]piperazine-1-carboxamide

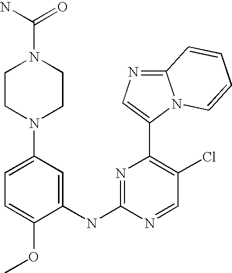

This compound was prepared following the procedure as described in Example 54 using 5-chloro-4-imidazo[1,2-a]pyridin-3-yl-N-(2-methoxy-5-piperazin-1-yl-phenyl)pyrimidin-2-amine (Example 3.10) and trimethylsilyl isocyanate as the starting materials (86%); NMR spectrum: 2.89-2.97 (m, 4H), 3.34-3.40 (m, 4H), 3.74 (s, 3H), 6.01 (s, 2H), 6.81 (dd, 1H), 6.94 (dd, 1H), 7.02 (d, 1H), 7.31 (d, 1H), 7.50 (dd, 1H), 7.77 (d, 1H), 8.54 (s, 1H), 8.71 (s, 1H), 8.85 (s, 1H), 8.57 (bs, 1H); Mass spectrum: 479 (MH+).

### Example 56

### (2S)-1-[4-[4-[(5-chloro-4-imidazo[1,2-a]pyridin-3-yl-pyrimidin-2-yl)amino]-3-methoxy-phenyl]piperazin-1-yl]-2,3-dihydroxy-propan-1-one

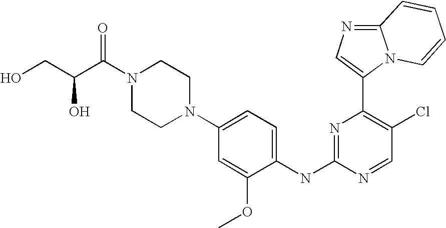

A mixture of 5-chloro-4-(imidazo[1,2-a]pyridin-3-yl)-N-(2-methoxy-4-(piperazin-1-yl)phenyl)pyrimidin-2-amine (Example 2, 120 mg, 0.28 mmol), N,N-diisopropylethylamine (0.096 mL, 0.55 mmol), 2-(3H-[1,2,3]triazolo[4,5-b]pyridin-3-yl)-1,1,3,3-tetramethylisouronium hexafluorophosphate(V) (209 mg, 0.55 mmol) and (S)-2,2-dimethyl-1,3-dioxolane-4-carboxylic acid (40.2 mg, 0.28 mmol) in dichloromethane (2 mL) was stirred at 25° C. for 1 hour. The reaction mixture was diluted with water and extracted with dichloromethane. The organic phase was dried over magnesium sulfate, concentrated and the residue was purified by flash chromatography on silica gel eluting with 0 to 10% methanol in dichloromethane to afford (4-(4-(5-chloro-4-(imidazo[1,2-a]pyridin-3-yl)pyrimidin-2-ylamino)-3-methoxyphenyl)piperazin-1-yl)((S)-2,2-dimethyl-1,3-dioxolan-4-yl)methanone (118 mg, 76%) as a yellow gum. This product was mixed with water (2 mL) and TFA (8 mL) and this mixture was stirred at 25° C. for 3 hours. After evaporation, the crude product was purified by preparative HPLC using a Waters X-Bridge reverse-phase column (5 microns silica, 19 mm diameter, 100 mm length) and decreasingly polar mixtures of water (containing 0.2% ammonium carbonate) and acetonitrile as eluent to afford (2S)-1-(4-(4-(5-chloro-4-(imidazo[1,2-a]pyridin-3-yl)pyrimidin-2-ylamino)-3-methoxyphenyl)piperazin-1-yl)-2,3-dihydroxypropan-1-one (31 mg, 28%) as a yellow solid; NMR spectrum: 3.14-3.26 (m, 4H), 3.46-3.52 (m, 1H), 3.54-3.61 (m, 1H), 3.61-3.79 (m, 4H), 3.77 (s, 3H), 4.37-4.43 (m, 1H), 4.74 (t, 1H), 5.00 (d, 1H), 6.56 (dd, 1H), 6.75 (d, 1H), 6.89 (bs, 1H), 7.30 (d, 1H), 7.49 (ddd, 1H), 7.75 (d, 1H), 8.46 (s, 1H), 8.72 (s, 1H), 8.81 (s, 1H), 9.55 (bs, 1H); Mass spectrum: 524 (MH+).

### Example 57

### 4-[4-[(5-chloro-4-imidazo[1,2-a]pyridin-3-yl-pyrimidin-2-yl)amino]-3-methoxy-phenyl]-N,N-dimethyl-piperidine-1-carboxamide

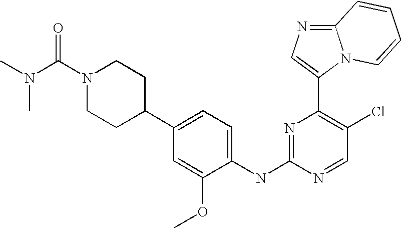

This compound was prepared following the procedure as described in Example 47.2 using 5-chloro-4-imidazo[1,2-a]pyridin-3-yl-N-[2-methoxy-4-(4-piperidyl)phenyl]pyrimidin-2-amine (Example 4) and dimethylamine as the starting materials (69%); NMR spectrum: 1.63 (dd, 1H), 1.69 dd, 1H), 1.77-1.86 (m, 2H), 2.67-2.76 (m, 1H), 2.77 (s, 6H), 2.77-2.85 (m, 2H), 3.66-3.73 (m, 2H), 3.80 (s, 3H), 6.86 (dd, 1H), 6.92 (dd, 1H), 7.02 (d, H), 7.47-7.54 (m, 2H), 7.76 (d, 1H), 8.50 (s, 1H), 8.73 (s, 1H), 8.88 (s, 1H), 9.60 (bs, 1H); Mass spectrum: 506 (MH+).

### Example 58

### 4-[4-[(5-chloro-4-imidazo[1,2-a]pyridin-3-yl-pyrimidin-2-yl)amino]-3-methoxy-phenyl]-N-(2-hydroxyethyl)-N-methyl-piperidine-1-carboxamide

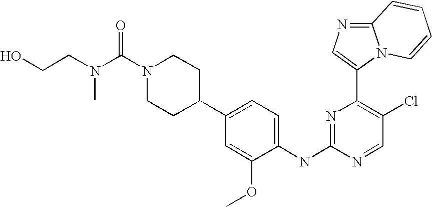

This compound was prepared following the procedure described for Example 47.1 using 5-chloro-4-imidazo[1,2-a]pyridin-3-yl-N-[2-methoxy-4-(4-piperidyl)phenyl]pyrimidin-2-amine (Example 4) and 2-(methylamino)ethanol as the starting materials (41%); NMR spectrum: 1.64 (dd, 1H), 1.69 (dd, 1H), 1.77-1.86 (m, 2H), 2.68-2.76 (m, 1H), 2.76-2.83 (m, 2H), 2.85 (s, 3H), 3.21 (t, 2H), 3.51-3.59 (m, 2H), 3.66-3.73 (m, 2H), 3.80 (s, 3H), 4.71 (t, 2H), 6.86 (dd, 1H), 6.92 (dd, 1H), 7.02 (d, 1H), 7.46-7.54 (m, 2H), 7.76 (d, 1H), 8.50 (s, 1H), 8.73 (s, 1H), 8.88 (s, 1H), 9.60 (bs, 1H); Mass spectrum: 536 (MH+).

### Example 59

A mixture of 3-(2,5-dichloropyrimidin-4-yl)imidazo[1,2-a]pyridine (see Method 1, 100 mg, 0.38 mmol), 1-(4-(4-amino-3-methoxyphenyl)-2-methylpiperidin-1-yl)ethanone (see Method 29, 99 mg, 0.38 mmol) and 4-methylbenzenesulfonic acid hydrate (71 mg, 0.38 mmol) in 2-pentanol (1.5 mL) was heated to 130° C. for 2 hours in a sealed tube. The reaction mixture was diluted in dichloromethane and washed with water. The organic layer was evaporated and the residue was purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent to afford 1-(4-(4-(5-chloro-4-(imidazo[1,2-a]pyridin-3-yl)pyrimidin-2-ylamino)-3-methoxyphenyl)-2-methylpiperidin-1-yl)ethanone (50 mg, 27%) and 5-chloro-4-(imidazo[1,2-a]pyridin-3-yl)-N-(2-methoxy-4-(2-methylpiperidin-4-yl)phenyl)pyrimidin-2-amine (10 mg, 5%).

### Example 59.1

### 1-[4-[4-[(5-chloro-4-imidazo[1,2-a]pyridin-3-yl-pyrimidin-2-yl)amino]-3-methoxy-phenyl]-2-methyl-1-piperidyl]ethanone

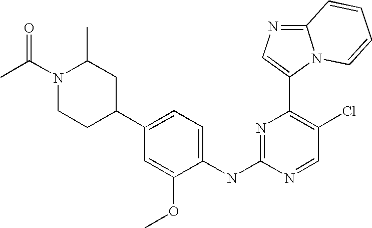

27%; NMR spectrum: 1.14 (d, 3H), 1.59-1.69 (m, 1H), 1.73-1.82 (m, 1H), 1.85-1.93 (m, 1H), 2.04 (s, 3H), 2.09-2.22 (m, 1H), 2.66-2.76 (m, 1H), 3.25 (bs, 1H), 3.76 (bd, 1H), 3.80 (s, 3H), 4.18 (bs, 1H), 6.87 (dd, 1H), 6.92 (dd, 1H), 7.05 (d, 1H), 7.45-7.54 (m, 2H), 7.76 (d, 1H), 8.50 (s, 1H), 8.73 (s, 1H), 8.88 (s, 1H), 9.60 (bs, 1H); Mass spectrum: 491 (MH+).

### Example 59.2

### 5-chloro-4-imidazo[1,2-a]pyridin-3-yl-N-[2-methoxy-4-(2-methyl-4-piperidyl)phenyl]pyrimidin-2-amine

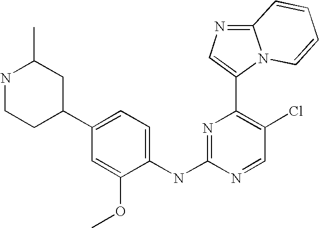

6%; NMR spectrum: 1.05 (d, 3H), 1.19-1.28 (m, 1H), 1.47-1.58 (m, 1H), 1.69-1.79 (m, 2H), 2.60-2.73 (m, 3H), 3.04-3.11 (m, 1H), 3.79 (s, 3H), 6.82 (dd, 1H), 6.91 (dd, 1H), 6.98 (d, 1H), 7.47 (d, 1H), 7.50 (dd, 1H), 7.76 (d, 1H), 8.50 (s, 1H), 8.76 (s, 1H), 8.87 (s, 1H), 9.60 (bs, 1H); Mass spectrum: 449 (MH+).

### Example 60

### 1-[3-[4-[(5-chloro-4-imidazo[1,2-a]pyridin-3-yl-pyrimidin-2-yl)amino]-3-methoxy-phenoxy]azetidin-1-yl]ethanone

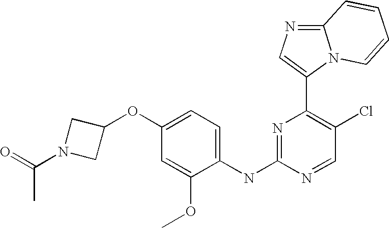

N-(4-(azetidin-3-yloxy)-2-methoxyphenyl)-5-chloro-4-(imidazo[1,2-a]pyridin-3-yl)pyrimidin-2-amine (Example 26, 48 mg, 0.11 mmol) and acetic anhydride (0.19 ml, 1.70 mmol) in dichloromethane (2 ml) were stirred at room temperature for 3 hours. After concentration the crude product was purified by preparative HPLC using a Waters X-Terra reverse-phase column (5 microns silica, 30 mm diameter, 150 mm length) and decreasingly polar mixtures of water (containing 0.2% ammonium carbonate) and acetonitrile as eluent to afford the title compound (40 mg, 76%); NMR spectrum: 1.81 (s, 3H), 3.77 (s, 3H), 3.79 (dd, 1H), 4.10 (dd, 1H), 4.31 (dd, 1H), 4.58 (dd, 1H), 5.05-5.13 (m, 1H), 6.44 (dd, 1H), 6.67 (d, 1H), 6.92 (bs, 1H), 7.38 (d, 1H), 7.50 (ddd, 1H), 7.76 (d, 1H), 8.47 (s, 1H), 8.72 (s, 1H), 8.89 (s, 1H), 9.55 (bs, 1H); Mass spectrum: 465 (MH+).

### Example 61

### (2S)-1-[3-[4-[(5-chloro-4-imidazo[1,2-a]pyridin-3-yl-pyrimidin-2-yl)amino]-3-methoxy-phenoxy]azetidin-1-yl]propan-2-ol

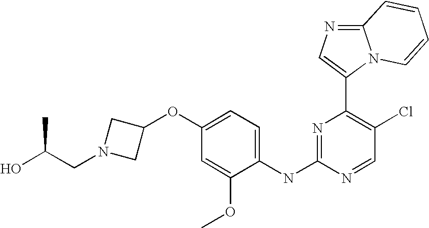

This compound was prepared following the procedure as described in Example 50 using N-(4-(azetidin-3-yloxy)-2-methoxyphenyl)-5-chloro-4-(imidazo[1,2-a]pyridin-3-yl)pyrimidin-2-amine (Example 26) and (R)-2-methyloxirane as the starting materials (61%); NMR spectrum: 1.03 (d, 3H), 2.37-2.46 (m, 2H), 3.00-3.09 (m, 2H), 3.55-3.63 (m, 1H), 3.75 (s, 3H), 3.78-3.84 (m, 2H), 4.38 (d, 1H), 4.80-4.88 (m, 1H), 6.42 (dd, 1H), 6.63 (d, 1H), 6.90 (bs, 1H), 7.33 (d, 1H), 7.49 (dd, 1H), 7.76 (d, 1H), 8.47 (s, 1H), 8.71 (s, 1H), 8.86 (s, 1H), 9.53 (bs, 1H); Mass spectrum: 481 (MH+).

### Example 62

### (2R)-1-[3-[4-[(5-chloro-4-imidazo[1,2-a]pyridin-3-yl-pyrimidin-2-yl)amino]-3-methoxy-phenoxy]azetidin-1-yl]propan-2-ol

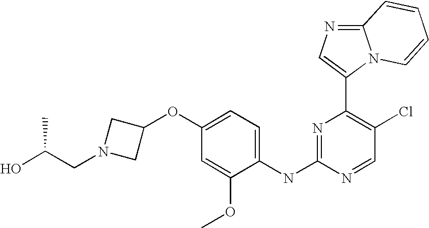

This compound was prepared following the procedure as described in Example 51 using N-(4-(azetidin-3-yloxy)-2-methoxyphenyl)-5-chloro-4-(imidazo[1,2-a]pyridin-3-yl)pyrimidin-2-amine (Example 26) and (R)-2-methyloxirane as the starting materials (59%); NMR spectrum: 1.03 (d, 3H), 2.37-2.46 (m, 2H), 3.01-3.08 (m, 2H), 3.55-3.63 (m, 1H), 3.75 (s, 3H), 3.78-3.84 (m, 2H), 4.38 (d, 1H), 4.81-4.88 (m, 1H), 6.42 (dd, 1H), 6.63 (d, 1H), 6.90 (bs, 1H), 7.33 (d, 1H), 7.49 (dd, 1H), 7.76 (d, 1H), 8.46 (s, 1H), 8.71 (s, 1H), 8.86 (s, 1H), 9.53 (bs, 1H); Mass spectrum: 481 (MH+).

### Example 63

### 1-[3-[4-[(5-chloro-4-imidazo[1,2-a]pyridin-3-yl-pyrimidin-2-yl)amino]-3-methoxy-phenoxy]azetidin-1-yl]-2-methyl-propan-2-ol

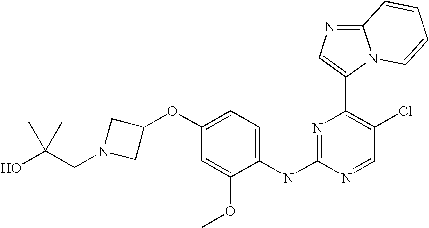

This compound was prepared following the procedure as described in Example 52 using N-(4-(azetidin-3-yloxy)-2-methoxyphenyl)-5-chloro-4-(imidazo[1,2-a]pyridin-3-yl)pyrimidin-2-amine (Example 26) and 2,2-dimethyloxirane as the starting materials (78%); NMR spectrum: 1.06 (s, 6H), 2.40 (s, 2H), 3.05-3.14 (m, 2H), 3.75 (s, 3H), 3.81-3.89 (m, 2H), 4.08 (s, 1H), 4.81-4.90 (m, 1H), 6.42 (dd, 1H), 6.63 (d, 1H), 6.90 (bs, 1H), 7.33 (d, 1H), 7.49 (dd, 1H), 7.76 (d, 1H), 8.46 (s, 1H), 8.72 (s, 1H), 8.86 (s, 1H), 9.52 (bs, 1H); Mass spectrum: 495 (MH+).

### Example 64

### 2-[3-[4-[(5-chloro-4-imidazo[1,2-a]pyridin-3-yl-pyrimidin-2-yl)amino]-3-methoxy-phenoxy]azetidin-1-yl]ethanol

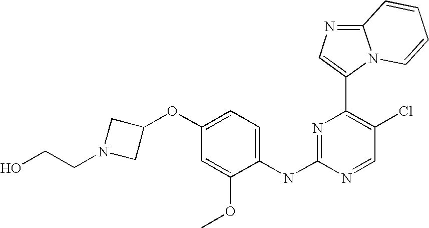

A mixture of 2-bromoethanol (0.030 mL, 0.43 mmol) and N,N-diisopropylethylamine (0.10 mL, 0.57 mmol) and N-(4-(azetidin-3-yloxy)-2-methoxyphenyl)-5-chloro-4-(imidazo[1,2-a]pyridin-3-yl)pyrimidin-2-amine (Example 26, 120 mg, 0.28 mmol) in DMF (1.2 mL) was stirred at 70° C. for 3 hours. After cooling, the reaction mixture was purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford 2-(3-(4-(5-chloro-4-(imidazo[1,2-a]pyridin-3-yl)pyrimidin-2-ylamino)-3-methoxyphenoxy)azetidin-1-yl)ethanol (35 mg, 26%) as a yellow solid; NMR spectrum: 2.54 (t, 2H), 3.01-3.08 (m, 2H), 3.36-3.43 (m, 2H), 3.75 (s, 3H), 3.77-3.83 (m, 2H), 4.43 (t, 1H), 4.80-4.88 (m, 1H), 6.42 (dd, 1H), 6.63 (d, 1H), 6.90 (bs, 1H), 7.33 (d, 1H), 7.50 (dd, 1H), 7.76 (d, 1H), 8.46 (s, 1H), 8.71 (s, 1H), 8.86 (s, 1H), 9.53 (bs, 1H); Mass spectrum: 467 (MH+).

### Example 65

### 2-[4-[4-[(5-chloro-4-imidazo[1,2-a]pyridin-3-yl-pyrimidin-2-yl)amino]-3-methoxy-phenyl]piperazin-1-yl]-N,N-dimethyl-acetamide

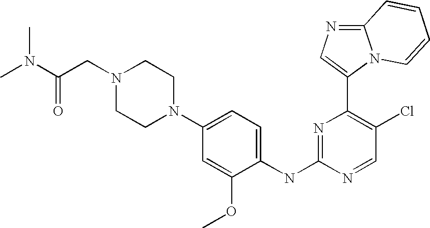

A suspension of 5-chloro-4-(imidazo[1,2-a]pyridin-3-yl)-N-(2-methoxy-4-(piperazin-1-yl)phenyl)pyrimidin-2-amine (Example 2, 115 mg, 0.25 mmol), cesium carbonate (82 mg, 0.25 mmol) and 2-chloro-N,N-dimethylacetamide (0.026 ml, 0.25 mmol) in DMF (1.5 ml) was stirred at 50° C. for 3 hours. The reaction mixture was concentrated to dryness, diluted with dichloromethane and the solution was washed with brine, dried (MgSO₄), filtered and evaporated to dryness. The crude product was purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent to afford 2-(4-(4-(5-chloro-4-(imidazo[1,2-a]pyridin-3-yl)pyrimidin-2-ylamino)-3-methoxyphenyl)piperazin-1-yl)-N,N-dimethylacetamide (54 mg, 41%) as a yellow solid; NMR spectrum: 2.59-2.66 (m, 4H), 2.83 (s, 3H), 3.05 (s, 3H), 3.16-3.24 (m, 4H), 3.21 (s, 2H), 3.75 (s, 3H), 6.53 (dd, 1H), 6.70 (d, 1H), 6.88 (bs, 1H), 7.27 (d, 1H), 7.50 (dd, 1H), 7.75 (d, 1H), 8.50 (s, 1H), 8.72 (s, 1H), 8.78 (s, 1H), 9.55 (bs, 1H); Mass spectrum: 521 (MH+).

### Example 66

### 2-[4-[4-[(5-chloro-4-imidazo[1,2-a]pyridin-3-yl-pyrimidin-2-yl)amino]-3-methoxy-phenyl]piperazin-1-yl]-N-methyl-acetamide

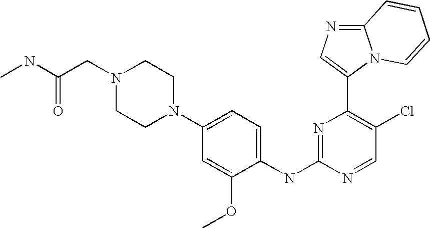

This compound was prepared following the procedure as described in Example 65 using 5-chloro-4-(imidazo[1,2-a]pyridin-3-yl)-N-(2-methoxy-4-(piperazin-1-yl)phenyl)pyrimidin-2-amine and 2-chloro-N-methylacetamide as the starting materials (31%); NMR spectrum: 2.57-2.63 (m, 4H), 2.64 (d, 3H), 2.98 (s, 2H), 3.19-3.27 (m, 4H), 3.75 (s, 3H), 6.54 (dd, 1H), 6.70 (d, 1H), 6.88 (bs, 1H), 7.27 (d, 1H), 7.50 (dd, 1H), 7.71-7.79 (m 2H), 8.55 (s, 1H), 8.72 (s, 1H), 8.79 (s, 1H), 9.55 (bs, 1H); Mass spectrum: 507 (MH+).

### Example 67

### 2-[4-[4-[(5-chloro-4-imidazo[1,2-a]pyridin-3-yl-pyrimidin-2-yl)amino]-3-methoxy-phenyl]piperazin-1-yl]-N-(2-hydroxyethyl)-N-methyl-acetamide

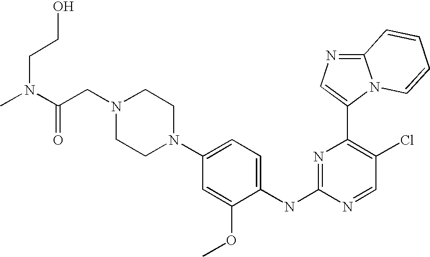

This compound was prepared following the procedure as described in Example 65 using 5-chloro-4-(imidazo[1,2-a]pyridin-3-yl)-N-(2-methoxy-4-(piperazin-1-yl)phenyl)pyrimidin-2-amine and 2-chloro-N-(2-hydroxyethyl)-N-methylacetamide as the starting materials (21%); NMR spectrum: 2.58-2.68 (m, 4H), 2.84 (s, 1.8H), 2.19 (s, 1.2H), 3.14-3.24 (m, 5H), 3.27 (s, 1.8H), 3.45-3.53 (m, 2H), 3.54-3.61 (m, 1.2H), 3.75 (s, 3H), 4.65 (t, 0.4H), 4.93 (t, 0.6H), 6.53 (d, 1H), 6.70 (s, 1H), 6.88 (bs, 1H), 7.27 (d, 1H), 7.50 (dd, 1H), 7.75 (d, 1H), 8.45 (s, 1H), 8.72 (s, 1H), 8.79 (s, 1H), 9.54 (bs, 1H); Mass spectrum: 551 (MH+).

### Example 68

### 3-[4-[(5-chloro-4-imidazo[1,2-a]pyridin-3-yl-pyrimidin-2-yl)amino]-3-methoxy-phenoxy]-N-(2-hydroxyethyl)-N-methyl-azetidine-1-carboxamide

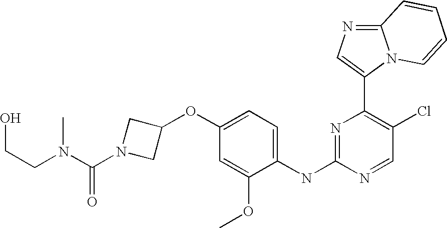

This compound was prepared following the procedure as described in Example 47.1 using N-(4-(azetidin-3-yloxy)-2-methoxyphenyl)-5-chloro-4-(imidazo[1,2-a]pyridin-3-yl)pyrimidin-2-amine (Example 26) and 2-(methylamino)ethanol as the starting materials (70%); NMR spectrum: 2.82 (s, 3H), 3.22 (t, 2H), 3.46-3.52 (m, 2H), 3.77 (s, 3H), 3.85-3.91 (m, 2H), 4.33-4.39 (m, 2H), 4.67 (t, 1H), 5.01-5.08 (m, 1H), 6.42 (dd, 1H), 6.66 (d, 1H), 6.92 (bs, 1H), 7.36 (d, 1H), 7.49 (dd, 1H), 7.76 (d, 1H), 8.47 (s, 1H), 8.72 (s, 1H), 8.88 (s, 1H), 9.54 (bs, 1H); Mass spectrum: 524 (MH+).

### Example 69

### 3-[4-[(5-chloro-4-imidazo[1,2-a]pyridin-3-yl-pyrimidin-2-yl)amino]-3-methoxy-phenoxy]-N,N-dimethyl-azetidine-1-carboxamide

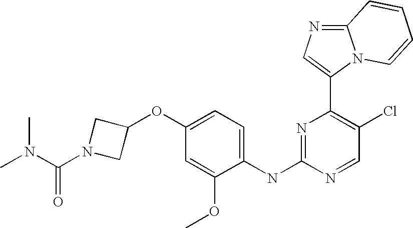

This compound was prepared following the procedure as described in Example 47.2 using N-(4-(azetidin-3-yloxy)-2-methoxyphenyl)-5-chloro-4-(imidazo[1,2-a]pyridin-3-yl)pyrimidin-2-amine (Example 26) and dimethylamine as the starting materials (77%); NMR spectrum: 2.78 (s, 6H), 3.76 (s, 3H), 3.83-3.91 (m, 1H), 4.32-4.41 (m, 2H), 5.01-5.09 (m, 1H), 6.42 (dd, 1H), 6.66 (d, 1H), 6.92 (bs, 1H), 7.36 (d, 1H), 7.49 (dd, 1H), 7.76 (d, 1H), 8.47 (s, 1H), 8.72 (s, 1H), 8.88 (s, 1H), 9.53 (bs, 1H); Mass spectrum: 494 (MH+).

### Example 70

### 2-[4-[(5-chloro-4-imidazo[1,2-a]pyridin-3-yl-pyrimidin-2-yl)amino]-3-methoxy-phenyl]propane-1,3-diol

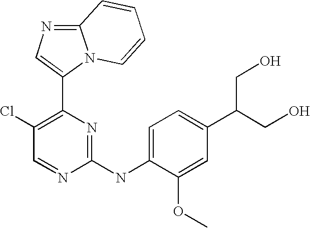

A mixture of 3-(2,5-dichloropyrimidin-4-yl)imidazo[1,2-a]pyridine (see Method 1, 150 mg, 0.54 mmol), 2-(4-amino-3-methoxyphenyl)propane-1,3-diol (see Method 38, 117 mg, 0.59 mmol) and p-toluenesulfonic acid (102 mg, 0.54 mmol) in 2-pentanol (1.5 mL) under argon was heated at 130° C. for 7 hours. The reaction mixture was then partitioned between dichloromethane and a saturated aqueous solution of sodium bicarbonate. The organic layer was dried (MgSO₄) and evaporated. The crude product was purified by flash chromatography on silica gel eluting with 0 to 12% MeOH/EtOAc to afford 2-(4-(5-chloro-4-(imidazo[1,2-a]pyridin-3-yl)pyrimidin-2-ylamino)-3-methoxyphenyl)propane-1,3-diol (145 mg, 63%) as a pale yellow solid; NMR spectrum: 2.82-2.90 (m, 1H), 3.61-3.68 (m, 2H), 3.71-3.79 (m, 2H), 3.77 (s, 3H), 4.60 (t, 2H), 6.85 (d, 1H), 6.95-7.04 (m, 2H), 7.43 (dd, 1H), 7.48 (d, 1H), 7.76 (d, 1H), 8.49 (s, 1H), 8.75 (s, 1H), 8.91 (s, 1H), 9.60 (bs, 1H); Mass spectrum: 426 (MH+).

### Example 71

### N-[4-[(3R)-3-aminopyrrolidin-1-yl]-2-methoxy-phenyl]-5-chloro-4-imidazo[1,2-a]pyridin-3-yl-pyrimidin-2-amine

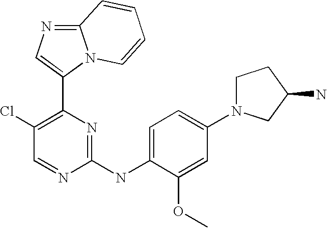

N-((3R)-1-(4-(5-chloro-4-(imidazo[1,2-a]pyridin-3-yl)pyrimidin-2-ylamino)-3-methoxyphenyl)pyrrolidin-3-yl)acetamide (Example 3.23, 209 mg, 0.44 mmol) in 6N hydrochloric acid (1.8 ml) was stirred at 80° C. for 4 hours. After cooling, the reaction mixture was concentrated and basified with a 2N aqueous solution of sodium hydroxide. The mixture was concentrated to dryness and the residue was purified by flash chromatography on silica gel eluting with 10 to 15% methanol in dichloromethane to afford N-(4-((R)-3-aminopyrrolidin-1-yl)-2-methoxyphenyl)-5-chloro-4-(imidazo[1,2-a]pyridin-3-yl)-pyrimidin-2-amine (146 mg, 77%); NMR spectrum: 1.83-1.92 (m, 1H), 2.15-2.25 (m, 1H), 3.07 (dd, 1H), 3.28-3.35 (m, 1H), 3.42-3.48 (m, 1H), 3.48-3.53 (m, 1H), 3.70-3.78 (m, 1H), 3.75 (s, 3H), 5.32 (bs, 2H), 6.14 (dd, 1H), 6.25 (d, 1H), 6.85 (bs, 1H), 7.17 (d, 1H), 7.47 (dd, 1H), 7.74 (d, 1H), 8.42 (s, 1H), 8.72 (s, 1H), 8.73 (s, 1H), 9.51 (bs, 1H); Mass spectrum: 436 (MH+).

### Examples 72 to 91

The following compounds were prepared from 3-(2-chloro-5-fluoropyrimidin-4-yl)imidazo[1,2-a]pyridine (Method 2) using the procedure indicated as the “reference example” in the following table. The amino-phenyl substituent used was as recited in the relevant reference example and 3-(2,5-dichloropyrimidin-4-yl)imidazo[1,2-a]pyridine in the reference examples was replaced by 3-(2-chloro-5-fluoropyrimidin-4-yl)imidazo[1,2-a]pyridine.

| | | | | % | Reference |
|---|---|---|---|---|---|
| Ex. No. | Name | MH+ | NMR spectrum | Yield | example |
| 72 | 1-[4-[4-[(5-fluoro-4- | 462 | 2.07 (s, 3H), 3.11-3.18 (m, | 27 | 1 |
| | imidazo[1,2-a]pyridin-3- | | 2H), 3.18-3.24 (m, 2H), | | |
| | yl-pyrimidin-2- | | 3.58-3.67 (m, 4H), 3.77 (s, | | |
| | yl)amino]-3-methoxy- | | 3H), 6.57 (d, 1H), 6.74 (s, | | |
| | phenyl]piperazin-1- | | 1H), 6.95 (bs, 1H), 7.37 (d, | | |
| | yl]ethanone | | 1H), 7.52 (dd, 1H), | | |
| | | | 7.78 (d, 1H), 8.36 (s, 1H), | | |
| | | | 8.44 (s, 1H), 8.64 (s, 1H), | | |
| | | | 9.78 (bs, 1H) | | |
| 73 | 5-fluoro-4-imidazo[1,2-a]pyridin- | 420 | 2.83-2.93 (m, 4H), | 50 | 2 |
| | 3-yl-N-(2- | | 3.07-3.14 (m, 4H), 3.75 (s, 3H), | | |
| | methoxy-4-piperazin-1- | | 6.53 (dd, 1H), 6.68 (d, | | |
| | yl-phenyl)pyrimidin-2- | | 1H), 6.93 (dd, 1H), | | |
| | amine | | 7.32 (d, 1H), 7.52 (ddd, 1H), | | |
| | | | 7.78 (d, 1H), 8.36 (d, 1H), | | |
| | | | 8.43 (d, 1H), 8.60 (s, 1H), | | |
| | | | 9.75 (bs, 1H) | | |
| 74 | 4-[4-[(5-fluoro-4-imidazo[1, | 527 | 2.50-2.55 (m partially | 39 | 3.7 |
| | 2-a]pyridin-3-yl- | | hidden by DMSOd6, 4H), | | |
| | pyrimidin-2-yl)amino]-3- | | 2.83 (s, 6H), 3.23-3.28 (m, | | |
| | methoxy-phenyl]-N,N- | | 4H), 3.77 (s, 3H), 6.58 (dd, | | |
| | dimethyl-piperazine-1- | | 1H), 6.74 (d, 1H), | | |
| | sulfonamide | | 6.96 (dd, 1H), 7.39 (d, 1H), | | |
| | | | 7.53 (ddd, 1H), 7.78 (d, | | |
| | | | 1H), 8.36 (d, 1H), 8.44 (d, | | |
| | | | 1H), 8.64 (s, 1H), 9.78 (bs, | | |
| | | | 1H) | | |
| 75 | 5-fluoro-4-imidazo[1,2-a]pyridin- | 420 | 2.76-2.84 (m, 4H), | 16 | 3.10 |
| | 3-yl-N-(2- | | 2.89-2.98 (m, 4H), 3.74 (s, 3H), | | |
| | methoxy-5-piperazin-1- | | 6.72 (dd, 1H), 7.99 (d, | | |
| | yl-phenyl)pyrimidin-2- | | 1H), 7.03 (dd, 1H), | | |
| | amine | | 7.36 (d, 1H), 7.54 (ddd, 1H), | | |
| | | | 7.80 (d, 1H), 8.38 (d, 1H), | | |
| | | | 8.52 (d, 1H), 8.68 (s, 1H), | | |
| | | | 9.82 (bs, 1H) | | |
| 76 | N-[4-[(3R)-3- | 448 | 1.80-1.91 (m, 1H), | 46 | 3.11 |
| | dimethylaminopyrrolidin- | | 2.15-2.23 (m, 1H), 2.24 (s, 6H), | | |
| | 1-yl]-2- | | 2.79-2.88 (m, 1H), | | |
| | methoxy-phenyl]-5- | | 3.07-3.14 (m, 1H), 3.27-3.33 (m | | |
| | fluoro-4-imidazo[1,2- | | partially hidden by H2O, | | |
| | a]pyridin-3-yl-pyrimidin- | | 1H), 3.40-3.46 (m, 1H), | | |
| | 2-amine | | 3.48-3.54 (m, 1H), 3.74 (s, | | |
| | | | 3H), 6.17 (dd, 1H), | | |
| | | | 6.28 (d, 1H), 6.88 (bs, 1H), | | |
| | | | 7.21 (d, 1H), 7.50 (ddd, 1H), | | |
| | | | 7.77 (d, 1H), 8.34 (d, 1H), | | |
| | | | 8.40 (d, 1H), 8.51 (s, 1H), | | |
| | | | 9.73 (bs, 1H) | | |
| 77 | 5-fluoro-4-imidazo[1,2-a]pyridin- | 449 | 1.46-1.58 (m, 2H), | 37 | 3.20 |
| | 3-yl-N-[2- | | 1.86-1.95 (m, 2H), | | |
| | methoxy-5-(4-methoxy- | | 2.73-2.81 (m, 2H), 3.25 (s, 3H), | | |
| | 1-piperidyl)phenyl]pyrimidin- | | 3.25-3.31 (m, 2H), | | |
| | 2- | | 3.35-3.42 (m partially hidden by | | |
| | amine | | H2O, 1H), 3.74 (s, 3H), | | |
| | | | 6.76 (dd, 1h), 6.98 (d, 1H), | | |
| | | | 7.02 (dd, 1H), 7.39 (d, | | |
| | | | 1H), 7.54 (ddd, 1H), | | |
| | | | 7.80 (d, 1H), 8.39 (d, 1H), | | |
| | | | 8.52 (d, 1H), 8.67 (s, 1H), | | |
| | | | 9.83 (bs, 1H) | | |
| 78 | 1-[4-[4-[(5-fluoro-4- | 461 | 1.47-1.59 (m, 1H), | 9 | 3.21 |
| | imidazo[1,2-a]pyridin-3- | | 1.62-1.74 (m, 1H), | | |
| | yl-pyrimidin-2- | | 1.79-1.89 (m, 2H), 2.05 (s, 3H), | | |
| | yl)amino]-3-methoxy- | | 2.56-2.65 (m, 1H), | | |
| | phenyl]-1- | | 2.76-2.85 (m, 1H), | | |
| | piperidyl]ethanone | | 3.11-3.19 (m, 1H), 3.80 (s, 3H), | | |
| | | | 3.92-3.99 (m, 1H), | | |
| | | | 4.54-4.61 (m, 1H), 6.87 (dd, | | |
| | | | 1H), 6.99 (dd, 1H), | | |
| | | | 7.01 (d, 1H), 7.54 (ddd, 1H), | | |
| | | | 7.57 (d, 1H), 7.79 (d, 1H), | | |
| | | | 8.39 (d, 1H), 8.49 (d, 1H), | | |
| | | | 8.72 (s, 1H), 9.84 (s, | | |
| | | | 0.5H), 9.85 (s, 0.5H) | | |
| 79 | 5-fluoro-4-imidazo[1,2-a]pyridin- | 421 | 3.14-3.20 (m, 4H), 3.76 (s, | 50 | 3.43 |
| | 3-yl-N-(2- | | 3H), 3.76-3.82 (m, 4H), | | |
| | methoxy-4-morpholino- | | 6.56 (dd, 1H), 6.71 (d, | | |
| | phenyl)pyrimidin-2- | | 1H), 6.95 (dd, 1H), | | |
| | amine | | 7.36 (d, 1H), 7.53 (ddd, 1H), | | |
| | | | 7.78 (d, 1H), 8.36 (d, 1H), | | |
| | | | 8.44 (d, 1H), 8.62 (s, 1H), | | |
| | | | 8.76 (bs, 1H) | | |
| 80 | 5-fluoro-4-imidazo[1,2-a]pyridin- | 405 | 1.96-2.06 (m, 4H), | 27 | 3.44 |
| | 3-yl-N-(2- | | 3.26-3.33 (m, 4H), 3.74 (s, 3H), | | |
| | methoxy-4-pyrrolidin-1- | | 6.17 (dd, 1H), 6.27 (d, | | |
| | yl-phenyl)pyrimidin-2- | | 1H), 6.87 (bs, 1H), 7.21 (d, | | |
| | amine | | 1H), 7.50 (ddd, 1H), | | |
| | | | 7.77 (d, 1H), 7.34 (d, 1H), | | |
| | | | 7.40 (d, 1H), 8.51 (s, 1H), | | |
| | | | 9.74 (bs, 1H) | | |
| 81 | 2-[4-[4-[(5-fluoro-4- | 464 | 2.46 (t, 2H), 2.56-2.64 (m, | 61 | 3.45 |
| | imidazo[1,2-a]pyridin-3- | | 4H), 3.15-3.23 (m, 4H), | | |
| | yl-pyrimidin-2- | | 3.52-3.59 (m, 2H), 3.75 (s, | | |
| | yl)amino]-3-methoxy- | | 3H), 4.45 (bs, 1H), | | |
| | phenyl]piperazin-1- | | 6.54 (dd, 1H), 6.69 (d, 1H), | | |
| | yl]ethanol | | 6.93 (dd, 1H), 7.33 (d, | | |
| | | | 1H), 7.52 (ddd, 1H), | | |
| | | | 7.78 (d, 1H), 8.36 (d, 1H), | | |
| | | | 8.43 (d, 1H), 8.60 (s, 1H), | | |
| | | | 9.76 (bs, 1H) | | |
| 82 | 5-fluoro-4-imidazo[1,2-a]pyridin- | 478 | 2.55 (t, 2H), 2.57-2.63 (m, | 14 | 3.46 |
| | 3-yl-N-[2- | | 4H), 3.15-3.22 (m, 4H), | | |
| | methoxy-4-[4-(2- | | 3.26 (s, 3H), 3.49 (t, 2H), | | |
| | methoxyethyl)piperazin- | | 3.75 (s, 3H), 6.54 (dd, 1H), | | |
| | 1-yl]phenyl]pyrimidin-2- | | 6.69 (d, 1H), 6.93 (dd, | | |
| | amine | | 1H), 7.33 (d, 1H), | | |
| | | | 7.52 (ddd, 1H), 7.78 (d, 1H), | | |
| | | | 8.36 (d, 1H), 8.43 (d, 1H), | | |
| | | | 8.60 (s, 1H), 8.75 (bs, 1H) | | |
| 83 | (3S)-1-[4-[(5-fluoro-4- | 421 | 1.90-1.98 (m, 1H), | 25 | 3.47 |
| | imidazo[1,2-a]pyridin-3- | | 3.15 (dd, 1H), 3.12-3.17 (m, | | |
| | yl-pyrimidin-2- | | 1H), 3.33-3.36 (m partially | | |
| | yl)amino]-3-methoxy- | | hidden by H2O, 1H), | | |
| | phenyl]pyrrolidin-3-ol | | 3.36-3.44 (m, 1H), 1.49 (dd, | | |
| | | | 1H), 3.74 (s, 3H), | | |
| | | | 4.42-4.49 (m, 1H), 5.00 (bs, | | |
| | | | 1H), 6.15 (dd, 1H), | | |
| | | | 6.25 (d, 1H), 6.88 (bs, 1H), | | |
| | | | 7.20 (d, 1H), 7.48 (dd, 1H), | | |
| | | | 7.76 (d, 1H), 8.34 (d, 1H), | | |
| | | | 8.40 (d, 1H), 8.51 (s, 1H), | | |
| | | | 9.70 (bs, 1H) | | |
| 84 | 5-fluoro-4-imidazo[1,2-a]pyridin- | 435 | 1.91-2.00 (m, 2H), | 32 | 3.48 |
| | 3-yl-N-[2- | | 3.59-3.68 (m, 6H), 3.74 (s, 3H), | | |
| | methoxy-4-(1,4- | | 3.75-3.81 (m, 2H), | | |
| | oxazepan-4-yl)phenyl]pyrimidin- | | 6.37 (dd, 1H), 6.45 (d, 1H), | | |
| | 2- | | 6.93 (bs, 1H), 7.21 (d, 1H), | | |
| | amine | | 7.50 (dd, 1H), 7.77 (d, | | |
| | | | 1H), 8.35 (d, 1H), 8.41 (d, | | |
| | | | 1H), 8.51 (s, 1H), 9.76 (bs, | | |
| | | | 1H) | | |
| 85 | (3R)-1-[4-[(5-fluoro-4- | 421 | 1.90-1.99 (m, 1H), | 16 | 3.49 |
| | imidazo[1,2-a]pyridin-3- | | 2.05-2.15 (m, 1H), | | |
| | yl-pyrimidin-2- | | 3.11-3.19 (m, 1H), 3.32-3.37 (m | | |
| | yl)amino]-3-methoxy- | | partially hidden by H2O, | | |
| | phenyl]pyrrolidin-3-ol | | 1H), 3.37-3.44 (m, 1H), | | |
| | | | 3.49 (dd, 1H), 3.74 (s, 3H), | | |
| | | | 4.42-4.48 (m, 1H), | | |
| | | | 5.00 (bs, 1H), 6.15 (dd, 1H), | | |
| | | | 6.25 (d, 1H), 6.88 (bs, 1H), | | |
| | | | 7.20 (d, 1H), 7.48 (dd, | | |
| | | | 1H), 7.76 (d, 1H), 8.34 (d, | | |
| | | | 1H), 8.40 (d, 1H), 8.51 (s, | | |
| | | | 1H), 9.70 (bs, 1H) | | |
| 86 | 5-fluoro-4-imidazo[1,2-a]pyridin- | 419 | 1.62-1.74 (m, 2H), | 7 | 4 |
| | 3-yl-N-[2- | | 1.80-1.88 (m, 2H), | | |
| | methoxy-4-(4- | | 2.66-2.80 (m, 3H), 3.14-3.21 (m, | | |
| | piperidyl)phenyl]pyrimidin- | | 2H), 3.80 (s, 3H), 6.85 (dd, | | |
| | 2-amine | | 1H), 6.94-7.03 (m, 2H), | | |
| | | | 7.54 (ddd, 1H), 7.58 (d, | | |
| | | | 1H), 7.80 (d, 1H), 8.39 (d, | | |
| | | | 1H), 8.49 (d, 1H), 8.74 (s, | | |
| | | | 1H), 9.84 (bs, 0.5H), | | |
| | | | 9.85 (bs, 0.5H) | | |
| 87 | 5-fluoro-4-imidazo[1,2-a]pyridin- | 435 | 1.47-1.55 (m, 1H), | 43 | 6.1 |
| | 3-yl-N-[2- | | 1.63-1.80 (m, 2H), | | |
| | methoxy-4-[[(2R)- | | 1.85-1.94 (m, 1H), 1.98 (bs, 1H), | | |
| | pyrrolidin-2-yl]methoxy]phenyl]pyrimidin- | | 2.79-2.93 (m, 2H), | | |
| | 2-amine | | 3.41-3.49 (m, 1H), 3.76 (s, 3H), | | |
| | | | 3.89 (d, 2H), 6.57 (dd, | | |
| | | | 1H), 6.71 (d, 1H), | | |
| | | | 6.96 (dd, 1H), 7.41 (d, 1H), | | |
| | | | 7.52 (dd, 1H), 7.79 (d, | | |
| | | | 1H), 8.37 (d, 1H), 8.44 (d, | | |
| | | | 1H), 8.67 (s, 1H), 9.67 (bs, | | |
| | | | 1H) | | |
| 88 | 4-[4-[(5-fluoro-4-imidazo[1, | 434 | (DMSOd6 + TFAd) | 15 | 18 |
| | 2-a]pyridin-3-yl- | | 3.37-3.44 (m, 2H), | | |
| | pyrimidin-2-yl)amino]-3- | | 3.52-3.58 (m, 2H), 3.83 (s, 3H), | | |
| | methoxy- | | 3.88 (s, 2H), 6.65 (dd, 1H), | | |
| | phenyl]piperazin-2-one | | 6.83 (d, 1H), 7.45 (d, 1H), | | |
| | | | 7.49 (bs, 1H), 8.10 (s, 1H), | | |
| | | | 8.11 (s, 1H), 8.66 (d, 1H), | | |
| | | | 8.95 (d, 1H), 10.10 (bs, 1H) | | |
| 89 | (2S)-1-[4-[4-[(5-fluoro-4- | 478 | 1.08 (d, 3H), 2.24 (dd, | 66 | 50 |
| | imidazo[1,2-a]pyridin-3- | | 1H), 2.32 (dd, 1H), | | |
| | yl-pyrimidin-2- | | 2.57-2.63 (m, 4H), | | |
| | yl)amino]-3-methoxy- | | 3.15-3.22 (m, 4H), 3.75 (s, 3H), | | |
| | phenyl]piperazin-1- | | 3.78-3.87 (m, 1H), 4.34 (d, | | |
| | yl]propan-2-ol | | 1H), 6.54 (dd, 1H), | | |
| | | | 6.69 (d, 1H), 6.93 (dd, 1H), | | |
| | | | 7.33 (d, 1H), 7.52 (ddd, | | |
| | | | 1H), 7.79 (d, 1H), 8.36 (d, | | |
| | | | 1H), 8.43 (d, 1H), 8.60 (s, | | |
| | | | 1H), 9.76 (bs, 1H) | | |
| 90 | (2R)-1-[4-[4-[(5-fluoro- | 478 | 1.08 (d, 3H), 2.24 (dd, | 64 | 51 |
| | 4-imidazo[1,2-a]pyridin- | | 1H), 2.32 (dd, 1H), | | |
| | 3-yl-pyrimidin-2- | | 2.57-2.63 (m, 4H), | | |
| | yl)amino]-3-methoxy- | | 3.15-3.22 (m, 4H), 3.75 (s, 3H), | | |
| | phenyl]piperazin-1- | | 3.78-3.87 (m, 1H), 4.34 (d, | | |
| | yl]propan-2-ol | | 1H), 6.54 (dd, 1H), | | |
| | | | 6.69 (d, 1H), 6.93 (dd, 1H), | | |
| | | | 7.33 (d, 1H), 7.52 (ddd, | | |
| | | | 1H), 7.79 (d, 1H), 8.36 (d, | | |
| | | | 1H), 8.43 (d, 1H), 8.60 (s, | | |
| | | | 1H), 9.76 (bs, 1H) | | |
| 91 | 1-[4-[4-[(5-fluoro-4- | 492 | 1.14 (s, 6H), 2.28 (s, 2H), | 65 | 52 |
| | imidazo[1,2-a]pyridin-3- | | 2.66-2.74 (m, 4H), | | |
| | yl-pyrimidin-2- | | 3.14-3.22 (m, 4H), 3.75 (s, 3H), | | |
| | yl)amino]-3-methoxy- | | 4.14 (s, 1H), 6.54 (dd, 1H), | | |
| | phenyl]piperazin-1-yl]-2- | | 6.68 (d, 1H), 6.94 (dd, | | |
| | methyl-propan-2-ol | | 1H), 7.33 (d, 1H), | | |
| | | | 7.52 (ddd, 1H), 7.78 (d, 1H), | | |
| | | | 8.36 (d, 1H), 8.43 (d, 1H), | | |
| | | | 8.59 (s, 1H), 9.77 (bs, 1H) | | |

### Example 81

### 2-[4-[4-[(5-fluoro-4-imidazo[1,2-a]pyridin-3-yl-pyrimidin-2-yl)amino]-3-methoxy-phenyl]piperazin-1-yl]ethanol

An alternative method for the preparation of 2-[4-[4-[(5-fluoro-4-imidazo[1,2-a]pyridin-3-yl-pyrimidin-2-yl)amino]-3-methoxy-phenyl]piperazin-1-yl]ethanol was conducted as follows:

2-(4-(4-amino-3-methoxyphenyl)piperazin-1-yl)ethanol (3.18 g, 12.6 mmol) and p-toluenesulfonic acid hydrate (4.59 g, 24.1 mmol) were added to a solution of 3-(2-chloro-5-fluoropyrimidin-4-yl)imidazo[1,2-a]pyridine (3.0 g, 12.0 mmol) in 4-methyl-2-pentanol (36 ml) and N-methyl-2-pyrrolidinone (18 ml). The reaction mixture was stirred at 160° C. for 6 hours under argon atmosphere. The mixture was allowed to cool to room temperature and a solid precipitate was formed. This suspension was diluted with ethyl acetate under stirring and the solid was filtered then washed with diethyl ether. The collected solid was dissolved in water and the resulting solution was basified with solid sodium bicarbonate. The mixture was extracted three times with methylene chloride and the organic phase was dried over MgSO₄, filtered and concentrated to give 7.3 g of crude material. The crude product was purified by flash chromatography on silica gel eluting with 0 to 22% methanol in a 1:1 mixture of methylene chloride and ethyl acetate. After evaporation of the collected fractions, the residue was dissolved in methylene chloride and ethanol. Partial concentration of this solution under reduced pressure allowed a solid to precipitate. Diethyl ether was added and the solid was filtered, washed with diethyl ether and dried under vacuum at 50° C. to afford 2-(4-(4-(5-fluoro-4-(imidazo[1,2-a]pyridin-3-yl)pyrimidin-2-ylamino)-3-methoxyphenyl)piperazin-1-yl)ethanol (3.42 g, 61%) as a pale yellow crystalline solid. The crystalline solid obtained was characterised by X-ray powder diffraction and Differential Scanning Calorimetry according to the procedures set out below. The X-ray powder diffraction showed the solid to have a crystalline form, Form A, which has an X-ray powder diffraction pattern as shown in FIG. 2. The ten most prominent peaks are as follows:

TABLE A
Ten most Prominent X-Ray Powder Diffraction peaks for
2-[4-[4-[(5-fluoro-4-imidazo[1,2-a]pyridin-3-yl-pyrimidin-2-yl)amino]-
3-methoxy-phenyl]piperazin-1-yl]ethanol Form A

| Angle 2- | | Relative |
|---|---|---|
| Theta (2θ) | Intensity % | Intensity |
| 22.395 | 100.0 | vs |
| 17.003 | 54.8 | vs |
| 15.169 | 54.7 | vs |
| 12.584 | 52.2 | vs |
| 14.407 | 40.7 | vs |
| 20.745 | 38.9 | vs |
| 25.178 | 35.3 | vs |
| 9.241 | 25.1 | vs |
| 26.435 | 25.1 | vs |
| 22.801 | 21.6 | s |

vs = very strong,
s = strong

The DSC analysis of the crystalline form, Form A, showed a single event with an onset at 171.3° C. and a peak at 172.0° C. Thus DSC analysis showed that the crystalline form, Form A, is a high melting solid with an onset of melting at about 171.3° C. and a peak at 172.0° C.

The material remained in this form when slurried in a variety of organic solvents, the slurries were conducted as follows: Approximately 20 mg of the original material was placed in a vial with a magnetic flea, and approximately 2 ml of a solvent (eg methanol) added, the vial was then sealed tightly with a cap and left to stir on a magnetic stirrer plate. After 3 days, the sample was removed from the plate, the cap taken off and the slurry left to dry under ambient conditions before it was analysed by XRPD and DSC.

When Form A material was slurried in solvents containing water Hydrate Form A was found.

The crystalline solid obtained was characterised by X-ray powder diffraction and Differential Scanning Calorimetry according to the procedures set out below. The X-ray powder diffraction showed the solid to have a crystalline form, Hydrate Form A, which has an X-ray powder diffraction pattern as shown in FIG. 2. The ten most prominent peaks are as follows:

| Angle 2- | | Relative |
|---|---|---|
| Theta (2θ) | Intensity % | Intensity |
| 5.222 | 100.0 | vs |
| 18.244 | 16.3 | s |
| 18.744 | 10.2 | s |
| 14.449 | 10.0 | s |
| 23.366 | 10.0 | s |
| 25.290 | 9.3 | m |
| 25.867 | 7.7 | m |
| 16.638 | 6.5 | m |
| 21.538 | 6.0 | m |
| 15.754 | 6.0 | m |

vs = very strong
s = strong
m = medium

DSC analysis of Hydrate Form A shows an initial endothermic event with an onset at 50.0° C. and a peak at 74.5° C. followed by a subsequent endothermic event with an onset of 80.6° C. and a peak at 83.8° C. This is then followed by a melt event with an onset at 170.6° C. and a peak at 171.8°.

An alternative method for the preparation of 2-[4-[4-[(5-fluoro-4-imidazo[1,2-a]pyridin-3-yl-pyrimidin-2-yl)amino]-3-methoxy-phenyl]piperazin-1-yl]ethanol was conducted as follows:

A solution of 3-(2-chloro-5-fluoropyrimidin-4-yl)imidazo[1,2-a]pyridine (200 g, 804.36 mmol) (Method 47), 2-(4-(4-amino-3-methoxyphenyl)piperazin-1-yl)ethanol (202 g, 804.36 mmol) (Method 48) and 4-methylbenzenesulfonic acid-monohydrate (306 g, 1608.73 mmol) in N-methyl-2-pyrrolidinone (1700 mL) was prepared by dissolving the 3-(2-chloro-5-fluoropyrimidin-4-yl)imidazo[1,2-a]pyridine in the N-methyl-2-pyrrolidinone, then adding the 2-(4-(4-amino-3-methoxyphenyl)piperazin-1-yl)ethanol followed by the 4-methylbenzenesulfonic acid-monohydrate (temperature increased approx 9° C.). The resulting solution was allowed to stir at 140° C. for 7 hours. The reaction mixture was allowed to cool to room temperature the solution becoming much thicker. To this solution was added water (2000 mL) (solution became much more mobile) before pouring, portionwise into a stirred solution of sat. aqueous NaHCO₃ (10000 mL) (Observed exotherm of 3° C.). This solution was diluted with water (25000 mL) giving rise to a beige precipitate before stirring at room temperature for 4 hours. This solution was filtered giving a green solid which was washed with water (3000 mL). The solid residue was dissolved in ˜20% MeOH (7000 mL) before drying (MgSO₄) and evaporating to a thick red/brown gum. The crude product was purified by flash silica chromatography, eluting with 7% MeOH in DCM. Pure fractions were evaporated to dryness to afford a yellow foam/red/brown gum. Trituration with ether gave a yellow solid, material slurried in ether (2500 mL) (on rotorvap) before filtering to give, after drying 2-(4-(4-(5-fluoro-4-(imidazo[1,2-a]pyridin-3-yl)pyrimidin-2-ylamino)-3-methoxyphenyl)piperazin-1-yl)ethanol (239.4 g, 64.2%) as a yellow powder.

A second batch of material was isolated through evaporating the ether trituration filtrate to a yellow solid. This material was stirred in ether (100 mL) for ˜4 hours before filtering and drying to give 2-(4-(4-(5-fluoro-4-(imidazo[1,2-a]pyridin-3-yl)pyrimidin-2-ylamino)-3-methoxyphenyl)piperazin-1-yl)ethanol (4.44 g, 1.192%) as a yellow powder.

### Examples 92 to 96

The following compounds were prepared from 3-(2-chloro-5-methoxypyrimidin-4-yl)imidazo[1,2-a]pyridine (Method 3) using the procedure indicated as the “reference example” in the following table. The amino-phenyl substituent used was as recited in the relevant reference example and 3-(2,5-dichloropyrimidin-4-yl)imidazo[1,2-a]pyridine in the reference examples was replaced by 3-(2-chloro-5-methoxypyrimidin-4-yl)imidazo[1,2-a]pyridine.

| | | | | % | Reference |
|---|---|---|---|---|---|
| Ex. No. | Name | MH+ | NMR spectrum | Yield | example |
| 92 | 1-[4-[4-[(4-imidazo[1,2-a]pyridin- | 474 | 2.07 (s, 3H), | 43 | 1 |
| | 3-yl-5-methoxy- | | 3.10-3.16 (m, 2H), 3.16-3.23 (m, | | |
| | pyrimidin-2-yl)amino]-3- | | 2H), 3.58-3.66 (m, 4H), | | |
| | methoxy-phenyl]piperazin- | | 3.78 (s, 3H), 3.97 (s, | | |
| | 1-yl]ethanone | | 3H), 6.56 (dd, 1H), | | |
| | | | 6.73 (d, 1H), 6.90 (ddd, 1H), | | |
| | | | 7.47 (ddd, 1H), 7.48 (d, | | |
| | | | 1H), 7.73 (ddd, 1H), | | |
| | | | 8.16 (s, 1H), 8.30 (s, | | |
| | | | 1H), 8.56 (s, 1H), | | |
| | | | 9.89 (bs, 0.5H), 9.91 (bs, 0.5H) | | |
| 93 | 4-imidazo[1,2-a]pyridin-3- | 432 | 2.34 (bs, 1H), | 59 | 2 |
| | yl-5-methoxy-N-(2- | | 2.82-2.92 (m, 4H), 3.04-3.14 (m, | | |
| | methoxy-4-piperazin-1-yl- | | 4H), 3.76 (s, 3H), | | |
| | phenyl)pyrimidin-2-amine | | 3.97 (s, 3H), 6.52 (dd, 1H), | | |
| | | | 6.66 (d, 1H), 6.88 (dd, | | |
| | | | 1H), 7.42 (d, 1H), | | |
| | | | 7.46 (ddd, 1H), 7.73 (d, 1H), | | |
| | | | 8.12 (s, 1H), 8.29 (s, | | |
| | | | 1H), 8.56 (s, 1H), | | |
| | | | 9.86 (s, 0.5H), 9.88 (s, 0.5H) | | |
| 94 | 1-[4-[4-[(4-imidazo[1,2-a]pyridin- | 473 | 1.51-1.59 (m, 1H), | 42 | 3.21 |
| | 3-yl-5-methoxy- | | 1.61-1.73 (m, 1H), | | |
| | pyrimidin-2-yl)amino]-3- | | 1.77-1.91 (m, 2H), | | |
| | methoxy-phenyl]-1- | | 2.05 (s, 3H), 2.56-2.65 (m, | | |
| | piperidyl]ethanone | | 1H), 2.73-2.84 (m, 1H), | | |
| | | | 3.09-3.20 (m, 1H), | | |
| | | | 3.82 (s, 3H), 3.91-3.99 (m, | | |
| | | | 1H), 3.99 (s, 3H), | | |
| | | | 4.53-4.62 (m, 1H), 6.84 (d, | | |
| | | | 1H), 6.96 (dd, 1H), | | |
| | | | 6.98 (s, 1H), 7.49 (dd, 1H), | | |
| | | | 7.72 (d, 1H), 7.75 (d, | | |
| | | | 1H), 8.24 (s, 1H), | | |
| | | | 8.35 (s, 1H), 8.59 (s, 1H), | | |
| | | | 9.97 (s, 0.5H), 9.98 (s, | | |
| | | | 0.5H) | | |
| 95 | 2-[4-[4-[(4-imidazo[1,2-a]pyridin- | 476 | 2.46 (t, 2H), | 20 | 3.45 |
| | 3-yl-5-methoxy- | | 2.56-2.63 (m, 4H), 3.15-3.21 (m, | | |
| | pyrimidin-2-yl)amino]-3- | | 4H), 3.53-3.59 (m, 2H), | | |
| | methoxy-phenyl]piperazin- | | 3.76 (s, 3H), 3.97 (s, | | |
| | 1-yl]ethanol | | 3H), 4.44 (t, 1H), | | |
| | | | 6.53 (dd, 1H), 6.68 (d, 1H), | | |
| | | | 6.88 (dd, 1H), 7.42 (d, | | |
| | | | 1H), 7.46 (ddd, 1H), | | |
| | | | 7.73 (ddd, 1H), 8.13 (s, | | |
| | | | 1H), 8.29 (s, 1H), | | |
| | | | 8.56 (s, 1H), 9.87 (s, 0.5H), | | |
| | | | 9.88 (s, 0.5H) | | |
| 96 | 4-imidazo[1,2-a]pyridin-3- | 447 | 1.43-1.53 (m, 1H), | 94 | 6.1 |
| | yl-5-methoxy-N-[2- | | 1.60-1.78 (m, 2H), | | |
| | methoxy-4-[[(2R)- | | 1.83-1.92 (m, 1H), | | |
| | pyrrolidin-2-yl]methoxy]phenyl]pyrimidin- | | 2.77-2.89 (m, 2H), | | |
| | 2-amine | | 3.37-3.45 (m, 1H), | | |
| | | | 3.76 (s, 3H), 3.81-3.90 (m, | | |
| | | | 2H), 3.97 (s, 3H), | | |
| | | | 6.56 (dd, 1H), 6.69 (d, 1H), | | |
| | | | 6.90 (ddd, 1H), | | |
| | | | 7.46 (ddd, 1H), 7.49 (d, 1H), | | |
| | | | 7.73 (ddd, 1H), 8.19 (s, | | |
| | | | 1H), 8.60 (s, 1H), | | |
| | | | 8.56 (s, 1H), 9.86 (s, 0.5H), | | |
| | | | 9.88 (s, 0.5H) | | |

### Examples 97 to 106

The following compounds were prepared from 3-(2-chloro-5-methylpyrimidin-4-yl)imidazo[1,2-a]pyridine (Method 5) using the procedure as indicated as the “reference example” in the following table. The amino-phenyl substituent used was as recited in the relevant reference example and 3-(2,5-dichloropyrimidin-4-yl)imidazo[1,2-a]pyridine in the reference examples was replaced by 3-(2-chloro-5-methylpyrimidin-4-yl)imidazo[1,2-a]pyridine.

| | | | | % | Reference |
|---|---|---|---|---|---|
| Ex. No. | Name | MH+ | NMR spectrum | Yield | example |
| 97 | 1-[4-[4-[(4- | 458 | 2.06 (s, 3H), 2.37 (s, 3H), | 55 | 1 |
| | imidazo[1,2-a]pyridin- | | 3.08-3.15 (m, 2H), | | |
| | 3-yl-5-methyl- | | 3.15-3.22 (m, 2H), 3.56-3.66 (m, | | |
| | pyrimidin-2-yl)amino]- | | 4H), 3.79 (s, 3H), 6.53 (dd, | | |
| | 3-methoxy-phenyl]piperazin- | | 1H), 6.73 (d, 1H), 6.90 (dd, | | |
| | 1- | | 1H), 7.44 (ddd, 1H), 7.46 (d, | | |
| | yl]ethanone | | 1H), 7.72 (s, 1H), 8.23 (s, | | |
| | | | 1H), 8.26 (s, 1H), 8.30 (s, | | |
| | | | 1H), 9.60 (bs, 1H) | | |
| 98 | 4-imidazo[1,2- | 416 | 2.30 (bs, 1H), 2.37 (s, 3H), | 60 | 2 |
| | a]pyridin-3-yl-N-(2- | | 2.81-2.93 (m, 4H), | | |
| | methoxy-4-piperazin- | | 3.03-3.14 (m, 4H), 3.78 (s, 3H), | | |
| | 1-yl-phenyl)-5-methyl- | | 6.50 (dd, 1H), 6.67 (d, 1H), | | |
| | pyrimidin-2-amine | | 6.98 (dd, 1H), 7.41 (d, 1H), | | |
| | | | 7.47 (ddd, 1H), 7.72 (d, 1H), | | |
| | | | 8.21 (s, 1H), 8.26 (s, 1H), | | |
| | | | 8.30 (s, 1H), 9.60 (bs, 1H) | | |
| 99 | 1-[4-[4-[(4- | 457 | 1.45-1.56 (m, 1H), | 21 | 3.21 |
| | imidazo[1,2-a]pyridin- | | 1.60-1.71 (m, 1H), 1.77-1.86 (m, | | |
| | 3-yl-5-methyl- | | 2H), 2.05 (s, 3H), 2.39 (s, | | |
| | pyrimidin-2-yl)amino]- | | 3H), 2.55-2.63 (m, 1H), | | |
| | 3-methoxy-phenyl]-1- | | 2.72-2.81 (m, 1H), | | |
| | piperidyl]ethanone | | 3.09-3.17 (m, 1H), 3.83 (s, 3H), | | |
| | | | 3.90-3.98 (m, 1H), | | |
| | | | 4.52-4.51 (m, 1H), 6.81 (dd, 1H), | | |
| | | | 6.94 (dd, 1H), 6.99 (d, 1H), | | |
| | | | 7.45 (ddd, 1H), 7.73 (d, 1H), | | |
| | | | 7.74 (ddd, 1H), 8.27 (s, 2H), | | |
| | | | 8.67 (s, 1H), 9.63 (s, 0.5H), | | |
| | | | 9.64 (s, 0.5H) | | |
| 100 | 4-imidazo[1,2- | 417 | 2.36 (s, 3H), 3.11-3.16 (m, | 58 | 3.43 |
| | a]pyridin-3-yl-N-(2- | | 4H), 3.74-3.80 (m, 4H), | | |
| | methoxy-4- | | 3.78 (s, 3H), 6.52 (dd, 1H), | | |
| | morpholino-phenyl)-5- | | 6.70 (d, 1H), 6.89 (dd, 1H), | | |
| | methyl-pyrimidin-2- | | 7.41-7.47 (m, 2H), 7.72 (d, 1H), | | |
| | amine | | 8.22 (s, 1H), 8.26 (s, 1H), | | |
| | | | 8.30 (s, 1H), 9.60 (bs, 1H) | | |
| 101 | 2-[4-[4-[(4- | 460 | 2.36 (s, 3H), 2.43 (t, 2H), | 86 | 3.45 |
| | imidazo[1,2-a]pyridin- | | 2.56-2.61 (m, 4H), | | |
| | 3-yl-5-methyl- | | 3.13-3.19 (m, 4H), 3.55 (t, 2H), | | |
| | pyrimidin-2-yl)amino]- | | 3.77 (s, 3H), 4.46 (bs, 1H), | | |
| | 3-methoxy-phenyl]piperazin- | | 6.50 (dd, 1H), 6.68 (d, 1H), | | |
| | 1-yl]ethanol | | 6.88 (dd, 1H), 7.41 (d, 1H), | | |
| | | | 7.44 (ddd, 1H), 7.71 (d, 1H), | | |
| | | | 8.21 (s, 1H), 8.25 (s, 1H), | | |
| | | | 9.27 (s, 1H), 9.59 (bs, 0.5H), | | |
| | | | 9.60 (bs, 0.5H) | | |
| 102 | 4-imidazo[1,2- | 431 | 1.43-1.53 (m, 1H), | 9 | 6.1 |
| | a]pyridin-3-yl-N-[2- | | 1.59-1.77 (m, 2H), 1.82-1.91 (m, | | |
| | methoxy-4-[[(2R)- | | 1H), 1.94 (bs, 1H), 2.36 (s, | | |
| | pyrrolidin-2-yl]methoxy]phenyl]- | | 3H), 2.77-2.90 (m, 2H), | | |
| | 5- | | 3.37-3.45 (m, 1H), 3.76 (s, | | |
| | methyl-pyrimidin-2- | | 3H), 3.79-3.90 (m, 2H), | | |
| | amine | | 6.53 (dd, 1H), 6.70 (d, 1H), | | |
| | | | 6.90 (dd, 1H), 7.43 (d, 1H), | | |
| | | | 7.48 (d, 1H), 7.72 (d, 1H), | | |
| | | | 8.25 (s, 1H), 8.27 (s, 1H), 8.31 (s, | | |
| | | | 1H), 9.58 (bs, 1H) | | |
| 103 | 1-[4-[4-[(4- | 473 | 1.48-1.58 (m, 1H), | 54 | 7 |
| | imidazo[1,2-a]pyridin- | | 1.59-1.69 (m, 1H), 1.86-1.94 (m, | | |
| | 3-yl-5-methyl- | | 1H), 1.94-2.02 (m, 1H), | | |
| | pyrimidin-2-yl)amino]- | | 2.03 (s, 3H), 2.37 (s, 3H), | | |
| | 3-methoxy-phenoxy]- | | 3.23-3.31 (m, 1H), 2.33-2.40 \|(m | | |
| | 1-piperidyl]ethanone | | partially hidden by H2O, | | |
| | | | 1H), 3.65-3.73 (m, 1H), | | |
| | | | 3.78 (s, 3H), 3.82-3.90 (m, 1H), | | |
| | | | 4.59-4.67 (m, 1H), 6.61 (dd, | | |
| | | | 1H), 6.74 (d, 1H), 6.89 (dd, | | |
| | | | 1H), 7.44 (ddd, 1H), 7.50 (d, | | |
| | | | 1H), 7.72 (d, 1H), 8.26 (s, | | |
| | | | 1H), 8.29 (s, 1H), 8.31 (s, | | |
| | | | 1H), 9.58 (bs, 0.5H), | | |
| | | | 9.59 (bs, 0.5H), | | |
| 104 | (2S)-1-[4-[4-[(4- | 474 | 1.08 (d, 3H), 2.23 (dd, 1H), | 67 | 50 |
| | imidazo[1,2-a]pyridin- | | 2.32 (dd, 1H), 2.36 (s, 3H), | | |
| | 3-yl-5-methyl- | | 2.55-2.62 (m, 4H), | | |
| | pyrimidin-2-yl)amino]- | | 3.12-3.19 (m, 4H), 3.77 (s, 3H), | | |
| | 3-methoxy- | | 3.78-3.85 (m, 1H), 4.33 (d, | | |
| | phenyl]piperazin-1-yl]propan- | | 1H), 6.50 (dd, 1H), 6.68 (d, | | |
| | 2-ol | | 1H), 6.88 (dd, 1H), 7.41 (d, | | |
| | | | 1H), 7.44 (ddd, 1H), 7.71 (s, | | |
| | | | 1H), 8.21 (s, 1H), 8.25 (s, | | |
| | | | 1H), 8.30 (s, 1H), 9.60 (s, | | |
| | | | 0.5H), 9.61 (s, 0.5H) | | |
| 105 | (2R)-1-[4-[4-[(4- | 474 | 1.08 (d, 3H), 2.23 (dd, 1H), | 66 | 51 |
| | imidazo[1,2-a]pyridin- | | 2.32 (dd, 1H), 2.36 (s, 3H), | | |
| | 3-yl-5-methyl- | | 2.55-2.62 (m, 4H), | | |
| | pyrimidin-2-yl)amino]- | | 3.12-3.19 (m, 4H), 3.77 (s, 3H), | | |
| | 3-methoxy- | | 3.78-3.85 (m, 1H), 4.33 (d, | | |
| | phenyl]piperazin-1-yl]propan- | | 1H), 6.50 (dd, 1H), 6.68 (d, | | |
| | 2-ol | | 1H), 6.88 (dd, 1H), 7.41 (d, | | |
| | | | 1H), 7.44 (ddd, 1H), 7.71 (s, | | |
| | | | 1H), 8.21 (s, 1H), 8.25 (s, | | |
| | | | 1H), 8.29 (s, 1H), 9.59 (s, | | |
| | | | 0.5H), 9.60 (s, 0.5H) | | |
| 106 | 1-[4-[4-[(4- | 488 | 1.12 (s, 6H), 2.27 (s, 2H), | 64 | 52 |
| | imidazo[1,2-a]pyridin- | | 2.36 (s, 3H), 2.65-2.74 (m, | | |
| | 3-yl-5-methyl- | | 4H), 3.11-3.19 (m, 4H), | | |
| | pyrimidin-2-yl)amino]- | | 3.77 (s, 3H), 4.13 (s, 1H), | | |
| | 3-methoxy-phenyl]piperazin- | | 6.49 (dd, 1H), 6.67 (d, 1H), | | |
| | 1-yl]-2- | | 6.89 (dd, 1H), 7.41 (d, 1H), | | |
| | methyl-propan-2-ol | | 7.44 (ddd, 1H), 7.71 (s, 1H), | | |
| | | | 8.20 (s, 1H), 8.25 (s, 1H), 8.30 (s, | | |
| | | | 1H), 9.60 (s, 0.5H), 9.61 (s, | | |
| | | | 0.5H) | | |

### Example 101

### 2-[4-[4-[(4-imidazo[1,2-a]pyridin-3-yl-5-methyl-pyrimidin-2-yl)amino]-3-methoxy-phenyl]piperazin-1-yl]ethanol

An alternative method for the preparation of 2-[4-[4-[(4-imidazo[1,2-a]pyridin-3-yl-5-methyl-pyrimidin-2-yl)amino]-3-methoxy-phenyl]piperazin-1-yl]ethanol is as follows:

2-(4-(4-amino-3-methoxyphenyl)piperazin-1-yl)ethanol (2.15 g, 8.58 mmol) and p-toluenesulfonic acid hydrate (3.11 g, 16.3 mmol) were added to a solution of 3-(2-chloro-5-methylpyrimidin-4-yl)imidazo[1,2-a]pyridine (2.0 g, 8.17 mmol) in 4-methyl-2-pentanol (24 ml) and N-methyl-2-pyrrolidinone (12 ml). The reaction mixture was stirred at 160° C. for 6 hours under argon atmosphere. The mixture was allowed to cool to room temperature and a solid precipitated. This suspension was diluted with ethyl acetate under stirring and the solid was filtered then washed with cold ethyl acetate. The collected solid was dissolved in water and the resulting solution was basified with solid sodium bicarbonate. The mixture was extracted three times with methylene chloride and the organic phase was dried over MgSO₄, filtered and concentrated to give 4.6 g of crude material. The crude product was purified by flash chromatography on silica gel eluting with 0 to 5% methanol in methylene chloride. After evaporation of the collected fractions, the residue was dissolved in methylene chloride and ethanol. Partial concentration of this solution under reduced pressure allowed a solid to precipitate. Diethyl ether was added and the solid was filtered, washed with diethyl ether and dried under vacuum at 50° C. to afford 2-(4-(4-(4-(imidazo[1,2-a]pyridin-3-yl)-5-methylpyrimidin-2-ylamino)-3-methoxyphenyl)piperazin-1-yl)ethanol (1.60 g, 42%) as an off-white crystalline solid. The crystalline solid obtained was characterised by X-ray powder diffraction and Differential Scanning Calorimetry (DSC) according to the procedures set out below. The X-ray powder diffraction showed the solid to have a crystalline form, Form A, which has an X-ray powder diffraction pattern as shown in FIG. 3. The ten most prominent peaks are as follows:

| Angle 2- | | Relative |
|---|---|---|
| Theta (2θ) | Intensity % | Intensity |
| 24.450 | 100.0 | vs |
| 21.065 | 72.0 | vs |
| 10.506 | 50.4 | vs |
| 9.767 | 40.6 | vs |
| 19.525 | 39.3 | vs |
| 4.904 | 33.3 | vs |
| 11.868 | 29.6 | vs |
| 17.777 | 20.4 | s |
| 26.827 | 20.4 | s |
| 24.919 | 19.3 | s |

vs = very strong,
s = strong

The DSC analysis of the crystalline form, Form A, showed a single event with an onset at 182.1° C. and a peak at 184.1° C. Thus DSC analysis showed that the crystalline form, Form A, is a high melting solid with an onset of melting at about 182.1° C. and a peak at about 184.1° C.

### Examples 107 to 110

The following compounds were prepared from 3-(2-chloro-5-trifluoromethylpyrimidin-4-yl)imidazo[1,2-a]pyridine (Method 6) using the procedure as indicated as the “reference example” in the following table. The amino-phenyl substituent used was as recited in the relevant reference example and 3-(2,5-dichloropyrimidin-4-yl)imidazo[1,2-a]pyridine in the reference examples was replaced by 3-(2-chloro-5-trifluoromethylpyrimidin-4-yl)imidazo[1,2-a]pyridine.

| | | | | % | Reference |
|---|---|---|---|---|---|
| Ex. No. | Name | MH+ | NMR spectrum | Yield | example |
| 107 | 1-[4-[4-[[4- | 512 | 2.05 (s, 3H), 3.12-3.18 (m, | 48 | 1 |
| | imidazo[1,2-a]pyridin- | | 2H), 3.18-3.23 (m, 2H), | | |
| | 3-yl-5- | | 3.56-3.64 (m, 4H), 3.78 (s, | | |
| | (trifluoromethyl)pyrimidin- | | 3H), 6.55 (dd, 1H), 6.73 (bs, | | |
| | 2- | | 1H), 6.89 (bs, 1H), 7.26 (bs, | | |
| | yl]amino]-3-methoxy- | | 1H), 7.48 (bs, 1H), 7.74 (d, | | |
| | phenyl]piperazin-1- | | 1H), 8.12 (s, 1H), 8.73 (s, | | |
| | yl]ethanone | | 1H), 9.06 (bs, 1H) < 9.35 (s, | | |
| | | | 1H) | | |
| 108 | 1-[4-[4-[[4- | 511 | 1.45-1.57 (m, 1H), | 49 | 3.21 |
| | imidazo[1,2-a]pyridin- | | 1.60-1.72 (m, 1H), 1.75-1.86 (m, | | |
| | 3-yl-5- | | 2H), 2.04 (s, 3H), | | |
| | (trifluoromethyl)pyrimidin- | | 2.55-2.62 (m, 1H), 2.75-2.83 (m, 1H), | | |
| | 2- | | 3.09-3.18 (m, 1H), 3.80 (s, | | |
| | yl]amino]-3-methoxy- | | 3H), 3.90-3.98 (m, 1H), | | |
| | phenyl]-1- | | 4.52-4.60 (m, 1H), 6.85 (dd, | | |
| | piperidyl]ethanone | | 1H), 6.95 (bs, 1H), 7.04 (s, | | |
| | | | 1H), 7.43 (bs, 1H), 7.49 (dd, | | |
| | | | 1H), 7.75 (d, 1H), 8.12 (s, | | |
| | | | 1H), 8.77 (s, 1H), 9.20 (bs, | | |
| | | | 1H), 9.43 (s, 1H) | | |
| 109 | 2-[4-[4-[[4- | 514 | 2.46 (t, 2H), 2.56-2.61 (m, | 47 | 3.45 |
| | imidazo[1,2-a]pyridin- | | 4H), 3.14-3.18 (m partially | | |
| | 3-yl-5- | | hidden by H2O, 4H), | | |
| | (trifluoromethyl)pyrimidin- | | 3.53-3.59 (m, 2H), 3.77 (s, 3H), | | |
| | 2- | | 4.27 (t, 1H), 6.51 (dd, 1H), | | |
| | yl]amino]-3-methoxy- | | 6.67 (d, 1H), 6.90 (b, 1H), | | |
| | phenyl]piperazin- | | 7.27 (d, 1H), 7.46 (dd, 1H), | | |
| | 1-yl]ethanol | | 7.71 (d, 1H), 8.11 (s, 1H), | | |
| | | | 8.70 (s, 1H), 9.14 (s, 1H), | | |
| | | | 9.24 (bs, 1H) | | |
| 110 | 1-[4-[4-[[4- | 527 | 1.54 (bs, 1H), 1.64 (bs, 1H), | 49 | 7 |
| | imidazo[1,2-a]pyridin- | | 1.89 (bs, 1H), 1.97 (bs, 1H), | | |
| | 3-yl-5- | | 2.02 (s, 3H), 3.23-3.33 (m, | | |
| | (trifluoromethyl)pyrimidin- | | 1H), 3.33-3.41 (m, 1H), | | |
| | 2- | | 3.69 (bs, 1H), 3.77 (s, 3H), | | |
| | yl]amino]-3-methoxy- | | 3.43 (bs, 1H), 4.63 (bs, 1H), | | |
| | phenoxy]-1- | | 6.62 (d, 1H), 6.74 (s, 1H), | | |
| | piperidyl]ethanone | | 6.91 (bs, 1H), 7.35 (d, 1H), | | |
| | | | 7.47 (dd, 1H), 7.72 (d, 1H), | | |
| | | | 8.11 (s, 1H), 8.73 s, 1H), | | |
| | | | 9.22 (bs, 1H), 9.23 (s, 1H) | | |

### Example 111

### 1-[4-[(5-fluoro-4-imidazo[1,2-a]pyridin-3-yl-pyrimidin-2-yl)amino]-3-methoxy-phenyl]piperidin-4-ol

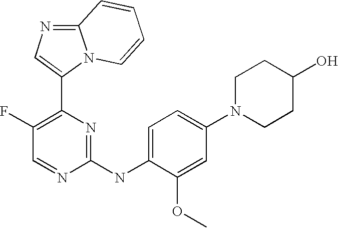

This compound was prepared from 3-(2-chloro-5-fluoropyrimidin-4-yl)imidazo[1,2-a]pyridine (Method 2) and 1-(4-amino-3-methoxyphenyl)piperidin-4-ol (see Method 39) following the procedure as described in Example 1 (40%); NMR spectrum: 1.48-1.58 (m, 2H), 1.82-1.91 (m, 2H), 2.84-2.93 (m, 2H), 3.54-3.62 (m, 2H), 3.62-3.70 (m, 1H), 3.75 (s, 3H), 4.71 (bs, 1H), 6.55 (dd, 1H), 6.68 (d, 1H), 6.93 (dd, 1H), 7.31 (d, 1H), 7.51 (ddd, 1H), 7.78 (d, 1H), 8.36 (d, 1H), 8.43 (d, 1H), 8.58 (s, 1H), 9.77 (bs, 1H); Mass spectrum: 435 (MH+).

### Example 112

### 5-fluoro-4-imidazo[1,2-a]pyridin-3-yl-N-[2-methoxy-5-(4-piperidyl)phenyl]pyrimidin-2-amine

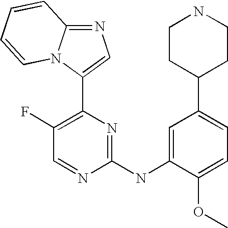

This compound was prepared from 3-(2-chloro-5-fluoropyrimidin-4-yl)imidazo[1,2-a]pyridine (Method 2) and tert-butyl 4-(3-amino-4-methoxyphenyl)piperidine-1-carboxylate (Method 30) following the procedure as described in Example 5 (34%); NMR spectrum: (DMSO-d6+TFAd) 1.74 (dd, 1H), 1.80 (dd, 1H), 1.90-1.98 (m, 2H), 2.77-2.83 (m, 1H), 2.95-3.05 (m, 2H), 3.33-3.41 (m, 2H), 3.82 (s, 3H), 7.06 (dd, 1H), 7.13 (d, 1H), 7.51 (dd, 1H), 7.58 (s, 1H), 8.07-8.15 (m, 2H), 8.70 (d, 1H), 8.96 (d, 1H), 10.14 (bs, 1H); Mass spectrum: 419 (MH+).

### Example 113

### 5-fluoro-4-imidazo[1,2-a]pyridin-3-yl-N-(2-methoxy-4-tetrahydropyran-4-yl-phenyl)pyrimidin-2-amine

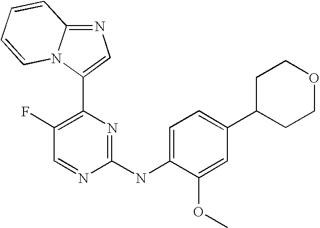

This compound was prepared from 3-(2-chloro-5-fluoropyrimidin-4-yl)imidazo[1,2-a]pyridine (Method 2) and 2-methoxy-4-(tetrahydro-2H-pyran-4-yl)aniline (see Method 40) following the procedure as described in Example 5 (25%); NMR spectrum: 1.70-1.81 (m, 4H), 2.77-2.86 (m, 1H), 3.42-3.51 (m, 2H), 3.80 (s, 3H), 3.96-3.99 (m, 1H), 3.99-4.01 (m, 1H), 6.88 (dd, 1H), 6.99 (dd, 1H), 7.01 (d, 1H), 7.53 (ddd, 1H), 7.57 (d, 1H), 7.80 (ddd, 1H), 8.38 (d, 1H) 8.49 (d, 1H), 8.73 (s, 1H), 9.84 (bs, 1H); Mass spectrum: 420 (MH+).

### Example 114

### 1-[(2R)-2-[[4-[(4-imidazo[1,2-a]pyridin-3-yl-5-methoxy-pyrimidin-2-yl)amino]-3-methoxy-phenoxy]methyl]pyrrolidin-1-yl]ethanone

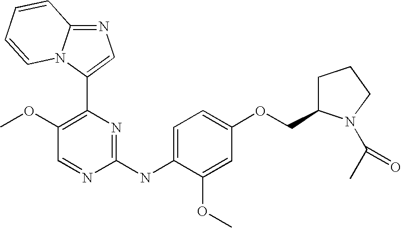

4-imidazo[1,2-a]pyridin-3-yl-5-methoxy-N-[2-methoxy-4-[[(2R)-pyrrolidin-2-yl]methoxy]phenyl]pyrimidin-2-amine (Example 96, 24 mg, 0.05 mmol) and acetic anhydride (60 μL, 0.54 mmol) in dichloromethane (0.2 mL) were stirred at room temperature for 30 minutes. After evaporation, the crude product was purified by flash chromatography on silica gel eluting with 0 to 5% methanolic ammonia (7N) in dichloromethane to afford 1-((2R)-2-((4-(4-(imidazo[1,2-a]pyridin-3-yl)-5-methoxypyrimidin-2-ylamino)-3-methoxyphenoxy)methyl)pyrrolidin-1-yl)ethanone (18 mg, 68%); NMR spectrum: 1.82-2.13 (m, 4H), 1.99 (s, 3H), 3.40-3.48 (m, 1H), 3.48-3.56 (m, 1H), 3.78 (s, 3H), 3.89 (dd, 1H), 3.97 (s, 3H), 4.16 (dd, 1H), 4.24-4.31 (m, 1H), 6.56 (dd, 1H), 6.81 (d, 1H), 6.89 (dd, 1H), 7.46 (ddd, 1H), 7.50 (d, 1H), 7.74 (d, 1H), 8.20 (s, 1H), 8.30 (s, 1H), 8.56 (s, 1H), 8.85 (bs, 0.5H), 8.86 (bs, 0.5H); Mass spectrum: 489 (MH+).

### Example 115

### 2-[4-[4-[(5-bromo-4-imidazo[1,2-a]pyridin-3-yl-pyrimidin-2-yl)amino]-3-methoxy-phenyl]piperazin-1-yl]ethanol

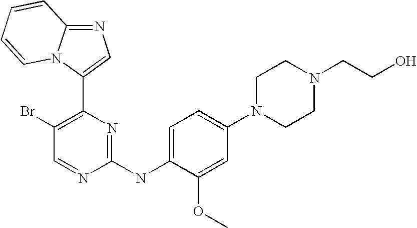

This compound was prepared from 3-(2-chloro-5-bromopyrimidin-4-yl)imidazo[1,2-a]pyridine (Method 4) and 2-[4-(4-amino-3-methoxyphenyl)piperazin-1-yl]ethanol as the starting materials following the procedure as described in Example 3.45 (41%); NMR spectrum: 2.45 (t, 2H), 2.59 (bs, 4H), 3.18 (bs, 4H), 3.50-3.61 (m, 2H), 3.76 (s, 3H), 4.45 (t, 1H), 6.51 (d, 1H), 6.69 (s, 1H), 6.88 (bs, 1H), 7.25 (d, 1H), 7.48 (dd, 1H), 7.74 (d, 1H), 8.54 (s, 1H), 8.76 (s, 1H), 8.77 (s, 1H), 9.41 (bs, 1H); Mass spectrum: 524 (MH+).

### Example 116

### 2-[[4-[4-(2-hydroxyethyl)piperazin-1-yl]-2-methoxy-phenyl]amino]-4-imidazo[1,2-a]pyridin-3-yl-pyrimidine-5-carbonitrile

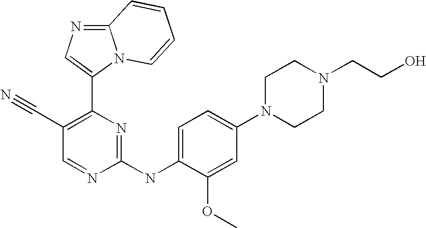

A mixture of 2-(4-(4-(5-bromo-4-(imidazo[1,2-a]pyridin-3-yl)pyrimidin-2-ylamino)-3-methoxyphenyl)piperazin-1-yl)ethanol (Example 115, 165 mg, 0.31 mmol), dicyanozinc (55 mg, 0.47 mmol), zinc dust (2.05 mg, 0.03 mmol), tris(dibenzylideneacetone)dipalladium (7.20 mg, 7.8 μmol) and (9,9-dimethyl-9H-xanthene-4,5-diyl)bis(diphenylphosphine) (18 mg, 0.03 mmol) in DMA (1 mL) was degassed with nitrogen and heated in a microwave reactor at 120° C. for 5 hours. The reaction mixture was purified by flash chromatography on silica gel eluting with 0 to 5% methanolic ammonia (7N) in dichloromethane to afford 2-(4-(4-(2-hydroxyethyl)piperazin-1-yl)-2-methoxyphenylamino)-4-(imidazo[1,2-a]pyridin-3-yl)pyrimidine-5-carbonitrile as a yellow solid (85 mg, 57%); NMR spectrum: (at 323° K) 2.48 (t partially hidden by DMSOd5, 2H), 2.56-2.65 (m, 4H), 3.18-3.28 (m, 4H), 3.53-3.59 (m, 2H), 3.75 (s, 3H), 4.28 (t, 1H), 6.56 (d, 1H), 6.69 (s, 1H), 6.81 (bs, 1H), 7.24 (bs, 1H), 7.54 (bs, 1H), 7.78 (d, 1H), 8.73 (s, 1H), 8.77 (s, 1H), 9.29 (bs, 1H), 9.46 (bs, 1H); Mass spectrum: 471 (MH+).

### Example 117

### 1-[4-[4-[(5-bromo-4-imidazo[1,2-a]pyridin-3-yl-pyrimidin-2-yl)amino]-3-methoxy-phenyl]piperazin-1-yl]ethanone

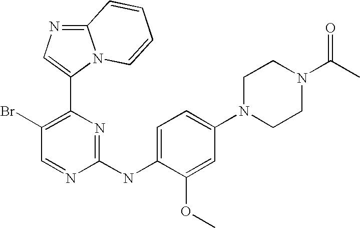

This compound was prepared from 3-(2-chloro-5-bromopyrimidin-4-yl)imidazo[1,2-a]pyridine (Method 4) and 1-(4-(4-amino-3-methoxyphenyl)piperazin-1-yl)ethanone as the starting materials following the procedure as described in Example 1 (32%); NMR spectrum: 2.06 (s, 3H), 3.11-3.18 (m, 2H), 3.18-3.23 (m, 2H), 3.57-3.65 (m, 4H), 3.77 (s, 3H), 6.54 (dd, 1H), 6.74 (d, 1H), 6.90 (bs, 1H), 7.30 (d, 1H), 7.47 (ddd, 1H), 7.74 (d, 1H), 8.55 (s, 1H), 8.77 (s, 1H), 8.78 (s, 1H), 9.42 (bs, 1H); Mass spectrum: 522 (MH+).

### Example 118

### 2-[[4-(4-acetylpiperazin-1-yl)-2-methoxy-phenyl]amino]-4-imidazo[1,2-a]pyridin-3-yl-pyrimidine-5-carbonitrile

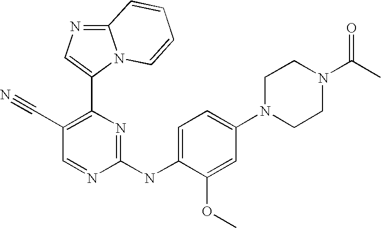

This compound was prepared from 1-[4-[4-[(5-bromo-4-imidazo[1,2-a]pyridin-3-yl-pyrimidin-2-yl)amino]-3-methoxy-phenyl]piperazin-1-yl]ethanone (Example 117) following the procedure as described in Example 116 (28%); NMR spectrum: (at 323° K) 2.08 (s, 3H), 3.18 (bs partially hidden by H2O, 2H), 3.26 (bs, 2H), 3.64 (bs, 4H), 3.78 (s, 3H), 6.60 (d, 1H), 6.72-6.79 (m, 1.5H), 6.86 (d, 0.5H), 6.92-6.98 (m, 0.5H), 7.29 (bs, 1H), 7.55 (bs, 1H), 7.64 (bs, 0.5H), 8.75 (s, 1H), 8.79 (s, 1H), 9.23 (bs, 1H), 9.51 (bs, 1H); Mass spectrum: 469 (MH+).

### Example 119

### 5-fluoro-4-imidazo[1,2-a]pyridin-3-yl-N-[2-methoxy-4-(4-methylpiperazin-1-yl)phenyl]pyrimidin-2-amine

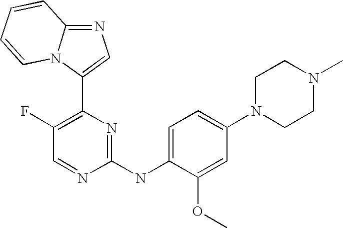

3-(2-chloro-5-fluoropyrimidin-4-yl)imidazo[1,2-a]pyridine (see Method 2, 175 mg, 0.70 mmol), 2-methoxy-4-(4-methylpiperazin-1-yl)aniline (see Method 32, 186 mg, 0.84 mmol) p-toluenesulfonic acid (267 mg, 1.40 mmol) were suspended in DMF (2.5 mL) and heated at 150° C. for 18 hours. The reaction mixture was allowed to cool to room temperature and purified by ion exchange chromatography, using an SCX column. The desired product was eluted from the column using 7M NH3/MeOH and evaporated to dryness to afford impure product. The crude product was purified by flash silica chromatography, elution gradient 0 to 4% 7M NH3/MeOH in dichloromethane. Fractions containing desired product were evaporated to dryness to afford impure material. The residue was then purified by preparative HPLC (Waters XBridge Prep C18 OBD column, 5μ silica, 21 mm diameter, 100 mm length), using decreasingly polar mixtures of water (containing 1% NH3) and MeCN as eluents. Fractions containing the desired compound were evaporated to dryness to afford the title compound (119 mg, 39%) as a yellow solid; NMR spectrum: (400 MHz) 2.26 (s, 3H), 3.20 (m, 4H), 3.76 (s, 3H), 6.54-6.57 (m, 1H), 6.70 (d, 1H), 6.94 (t, 1H), 7.35 (d, 1H), 7.51-7.55 (m, 1H), 7.77-7.80 (m, 1H), 8.37 (d, 1H), 8.44 (d, 1H), 8.57 (s, 1H), 9.75 (br s, 1H), 1×(m, 4H) obscured by DMSO signal; Mass spectrum: 434 (MH+).

### Example 120

### N-[4-(4-ethylpiperazin-1-yl)-2-methoxyphenyl]-5-fluoro-4-imidazo[1,2-a]pyridin-3-ylpyrimidin-2-amine

This compound was prepared following the procedure described for Example 119, using 4-(4-ethylpiperazin-1-yl)-2-methoxyaniline (see Method 32) and 3-(2-chloro-5-fluoropyrimidin-4-yl)imidazo[1,2-a]pyridine as the starting materials (29%); NMR spectrum: (400 MHz) 1.07 (t, 3H), 2.41 (q, 2H), 2.55 (m, 4H), 3.21 (m, 4H), 3.77 (s, 3H), 6.54-6.57 (m, 1H), 6.70-6.71 (d, 1H), 6.94 (t, 1H), 7.35 (d, 1H), 7.51-7.55 (m, 1H), 7.79 (d, 1H), 8.37 (d, 1H), 8.44 (d, 1H), 8.57 (s, 1H), 9.76 (br s, 1H); Mass spectrum: 448 (MH+).

### Examples 121 to 124

The following compounds were prepared using the procedure as described in Example 119 using 3-(2-chloro-5-bromopyrimidin-4-yl)imidazo[1,2-a]pyridine (Method 4) and the aniline indicated in the following table as the starting material.

| Ex. No. | Name | Starting aniline | MH+ | NMR spectrum | % Yield |
|---|---|---|---|---|---|
| 121 | 5-bromo-4-imidazo[1,2- | 2-methoxy-4- | 494 | (300 MHz) 2.37 (3H, s), | 29 |
| | a]pyridin-3-yl-N-[2- | (4-methylpiperazin- | | 2.61 (4H, t), 3.21 (4H, | |
| | methoxy-4-(4-methylpiperazin- | 1-yl)aniline | | t), 3.89 (3H, s), | |
| | 1-yl)phenyl]pyrimidin- | (Method 32) | | 6.52 (1H, dd), 6.58 (1H, d), | |
| | 2-amine | | | 6.84 (1H, td), | |
| | | | | 7.31-7.40 (2H, m), 7.73 (1H, dt), | |
| | | | | 7.99 (1H, d), 8.54 (1H, | |
| | | | | s), 8.84 (1H, s), | |
| | | | | 9.48 (1H, dt) | |
| 122 | 5-bromo-N-[2-ethoxy- | 2-ethoxy-4-(4- | 508 | (300 MHz) 1.45 (3H, t), | 36 |
| | 4-(4-methylpiperazin-1- | methylpiperazin- | | 2.37 (3H, s), 2.60 (4H, | |
| | yl)phenyl]-4- | 1-yl)aniline | | t), 3.19 (4H, t), | |
| | imidazo[1,2-a]pyridin- | (Method 33) | | 4.11 (2H, q), 6.50 (1H, dd), | |
| | 3-yl-pyrimidin-2-amine | | | 6.57 (1H, d), 6.83 (1H, | |
| | | | | td), 7.31-7.42 (2H, m), | |
| | | | | 7.74 (1H, dt), 8.01 (1H, | |
| | | | | d), 8.54 (1H, s), | |
| | | | | 8.84 (1H, s), 9.49 (1H, dt) | |
| 123 | 5-bromo-4-imidazo[1,2- | 2-methoxy-4- | 497 | (300 MHz) | 21 |
| | a]pyridin-3-yl-N-[2- | (1-methylpyrrolidin- | | 1.80-1.93 (1H, m), 2.37-2.51 (2H, | |
| | methoxy-4-(1-methylpyrrolidin- | 3- | | m), 2.37 (3H, s), | |
| | 3-yl)oxy- | yl)oxyaniline | | 2.65-2.81 (2H, m), 2.89 (1H, | |
| | phenyl]pyrimidin-2- | (Method 31) | | dd), 3.82 (3H, s), | |
| | amine | | | 4.95-5.03 (1H, m), 6.55 (1H, | |
| | | | | dd), 6.71 (1H, d), | |
| | | | | 6.96 (1H, t), 7.40 (1H, d), | |
| | | | | 7.53 (1H, t), 7.80 (1H, | |
| | | | | d), 8.62 (1H, s), | |
| | | | | 8.84 (2H, d), 9.47 (1H, s) | |
| 124 | 5-bromo-N-(4,5- | 4,5-dimethoxy- | 442 | (300 MHz) 2.13 (3H, s), | 19 |
| | dimethoxy-2-methyl- | 2-methyl- | | 3.69 (3H, s), 3.80 (3H, | |
| | phenyl)-4-imidazo[1,2- | aniline | | s), 6.73 (1H, s), | |
| | a]pyridin-3-yl- | | | 6.91 (1H, s), 6.96 (1H, s), | |
| | pyrimidin-2-amine | | | 7.46 (1H, t), 7.74 (1H, | |
| | | | | d), 8.57 (1H, s), | |
| | | | | 8.83 (1H, s), 9.11 (1H, s), | |
| | | | | 9.34 (1H, s) | |

### Example 125

### 1-[4-[4-[[5-chloro-4-[7-(hydroxymethyl)imidazo[1,2-a]pyridin-3-yl]pyrimidin-2-yl]amino]-3-methoxy-phenyl]piperazin-1-yl]ethanone

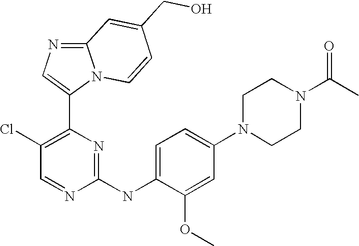

A mixture of (3-(2,5-dichloropyrimidin-4-yl)imidazo[1,2-a]pyridin-7-yl)methanol (Method 34, 400 mg, 1.36 mmol), 1-(4-(4-amino-3-methoxyphenyl)piperazin-1-yl)ethanone (Method 7, 338 mg, 1.36 mmol) and p-toluenesulfonic acid hydrate (258 mg, 1.36 mmol) in 2-pentanol (2 mL) was heated at 130° C. for 6 hours in a sealed tube. The reaction mixture was diluted in dichloromethane, washed with a saturated aqueous solution of sodium bicarbonate, dried and evaporated. The crude product was purified by flash chromatography on silica gel eluting with 0 to 10% methanol in dichloromethane to afford 1-(4-(4-(5-chloro-4-(7-(hydroxymethyl)imidazo[1,2-a]pyridin-3-yl)pyrimidin-2-ylamino)-3-methoxyphenyl)piperazin-1-yl)ethanone (250 mg, 36%) as a yellow solid; NMR spectrum: 2.07 (s, 3H), 3.14-3.19 (m, 2H), 3.20-3.25 (m, 2H), 3.58-3.65 (m, 4H), 3.76 (s, 3H), 4.60 (d, 2H), 5.51 (t, 1H), 6.56 (dd, 1H), 6.74 (d, 1H), 6.82 (bs, 1H), 7.29 (d, 1H), 7.60 (s, 1H), 8.43 (s, 1H), 8.69 (s, 1H), 8.79 (s, 1H), 9.46 (bs, 1H); Mass spectrum: 508 (MH+).

### Example 126

The following compounds were prepared using the procedure as described in Example 125 using 1-(4-(4-amino-3-methoxyphenyl)piperazin-1-yl)ethanone and the appropriate chloropyrimidines as set out in the following table. The chloropyrimidines were prepared as described in Method 34.

| | | | | | Chloro- |
|---|---|---|---|---|---|
| | | | | % | pyrimidine |
| Ex. No. | Name | MH+ | NMR spectrum | Yield | starting material |
| 126.1 | 1-[4-[4-[[4-(6- | 556 | 2.05 (s, 3H), | 47 | 6-bromo-3-(2,5- |
| | bromoimidazo[1,2-a]pyridin- | | 3.09-3.15 (m, 2H), 3.15-3.22 (m, | | dichloropyrimidin- |
| | 3-yl)-5- | | 2H), 3.54-3.63 (m, 4H), | | 4-yl)imidazo[1, |
| | chloropyrimidin-2- | | 3.78 (s, 3H), 6.55 (dd, | | 2-a]pyridine |
| | yl]amino]-3-methoxyphenyl]piperazin- | | 1H), 6.72 (d, 1H), | | |
| | 1-yl]ethanone | | 7.31 (d, 1H), 7.60 (dd, 1H), | | |
| | | | 7.74 (d, 1H), 8.50 (s, | | |
| | | | 1H), 8.66 (s, 1H), | | |
| | | | 8.81 (s, 1H), 9.61 (bs, 1H) | | |
| 126.2 | 3-[2-[[4-(4-acetylpiperazin- | 461 | 2.06 (s, 3H), | 31 | 3-(2,5-dichloropyrimidin- |
| | 1-yl)-2- | | 3.11-3.18 (m, 2H), 3.18-3.26 (m, | | 4-yl)imidazo[2, |
| | methoxyphenyl]amino]- | | 2H), 3.56-3.66 (m, 4H), | | 1-f]pyridine- |
| | 5-chloropyrimidin-4-yl]imidazo[2, | | 3.78 (s, 3H), 6.56 (dd, | | 6- |
| | 1-f]pyridine- | | 1H), 6.75 (d, 1H), | | carbonitrile |
| | 6-carbonitrile | | 6.93 (bs, 1H), 7.30 (d, 1H), | | |
| | | | 7.66 (dd, 1H), 8.46 (s, | | |
| | | | 1H), 8.71 (s, 1H), | | |
| | | | 8.84 (s, 1H), 9.64 (bs, 1H) | | |
| 126.3 | 1-[4-[4-[[5-chloro-4- | 514 | 2.06 (s, 3H), | 21 | 3-(2,5-dichloropyrimidin- |
| | (6,8- | | 3.09-3.16 (m, 2H), 3.16-3.21 (m, | | 4-yl)- |
| | difluoroimidazo[1,2-a]pyridin- | | 2H), 3.56-3.64 (m, 4H), | | 6,8-difluoroimidazo |
| | 3-yl)pyrimidin- | | 3.76 (s, 3H), 7.54 (dd, | | [1,2-a]pyridine |
| | 2-yl]amino]-3-methoxyphenyl]piperazin- | | 1H), 6.71 (d, 1H), | | |
| | 1-yl]ethanone | | 7.25 (d, 1H), 7.79 (dd, 1H), | | |
| | | | 8.51 (s, 1H), 8.77 (s, | | |
| | | | 1H), 8.99 (bs, 1H), | | |
| | | | 9.48 (bs, 1H) | | |
| 126.4 | 1-[4-[4-[[5-chloro-4-(6- | 492 | 2.06 (s, 3H), 2.15 (bs, | 34 | 3-(2,5-dichloropyrimidin- |
| | methylimidazo[1,2-a]pyridin- | | 3H), 3.10-3.16 (m, 2H), | | 4-yl)- |
| | 3-yl)pyrimidin- | | 3.16-3.22 (m, 2H), | | 6-methylimidazo[1, |
| | 2-yl]amino]-3-methoxyphenyl]piperazin- | | 3.55-3.63 (m, 4H), | | 2-a]pyridine |
| | 1- | | 3.74 (s, 3H), 6.54 (dd, 1H), | | |
| | yl]ethanone | | 6.71 (d, 1H), | | |
| | | | 7.31-7.37 (m, 2H), 7.66 (d, 1H), | | |
| | | | 8.45 (s, 1H), 8.68 (s, | | |
| | | | 1H), 8.73 (bs, 1H), | | |
| | | | 9.35 (bs, 1H) | | |
| 126.5 | 1-[4-[4-[[5-chloro-4-(6- | 521 | 2.05 (s, 3H), 2.68 (s, | 44 | 3-(2,5-dichloropyrimidin- |
| | dimethylaminoimidazo[1, | | 6H), 3.05-3.12 (m, 2H), | | 4-yl)- |
| | 2-a]pyridin-3- | | 3.12-3.17 (m, 2H), | | N,N-dimethylimidazo |
| | yl)pyrimidin-2- | | 3.54-3.62 (m, 4H), | | [2,1-f]pyridin- |
| | yl]amino]-3-methoxyphenyl]piperazin- | | 3.76 (s, 3H), 6.48 (dd, 1H), | | 6-amine |
| | 1- | | 6.68 (d, 1H), 7.41 (dd, | | |
| | yl]ethanone | | 1H), 7.51 (d, 1H), | | |
| | | | 7.62 (d, 1H), 8.45 (s, 1H), | | |
| | | | 8.47 (s, 1H), 8.49 (s, | | |
| | | | 1H), 8.85 (bs, 1H) | | |
| 126.6 | 1-[4-[4-[[5-chloro-4-(6- | 508 | 2.06 (s, 3H), | 48 | 3-(2,5-dichloropyrimidin- |
| | methoxyimidazo[1,2-a]pyridin- | | 3.06-3.03 (m, 2H), 3.13-3.20 (m, | | 4-yl)- |
| | 3-yl)pyrimidin- | | 2H), 3.53 (bs, 3H), | | 6- |
| | 2-yl]amino]-3-methoxyphenyl]piperazin- | | 3.55-3.64 (m, 4H), | | methoxyimidazo[1, |
| | 1- | | 3.75 (s, 3H), 6.49 (dd, 1H), | | 2-a]pyridine |
| | yl]ethanone | | 6.68 (d, 1H), 7.29 (dd, | | |
| | | | 1H), 7.40 (d, 1H), | | |
| | | | 7.69 (d, 1H), 8.48 (d, 1H), | | |
| | | | 8.57 (s, 1H), 8.67 (s, | | |
| | | | 1H) 9.16 (bs, 1H) | | |
| 126.7 | 3-[2-[[4-(4-acetylpiperazin- | 503 | 2.07 (s, 3H), | 28 | 3-(2,5-dichloropyrimidin- |
| | 1-yl)-2- | | 3.14-3.19 (m, 2H), 3.19-3.26 (m, | | 4- |
| | methoxyphenyl]amino]- | | 2H), 3.57-3.66 (m, 4H), | | yl)imidazo[1,2- |
| | 5-chloropyrimidin-4-yl]imidazo[1, | | 3.79 (s, 3H), 7.56 (dd, | | a]pyridine-7- |
| | 2-a]pyridine- | | 1H), 7.76 (d, 1H), | | carbonitrile |
| | 7-carbonitrile | | 7.22 (bs, 1H), 7.29 (d, 1H), | | |
| | | | 7.52 (s, 1H), 7.53 (s, | | |
| | | | 1H), 8.84 (s, 1H), | | |
| | | | 8.93 (s, 1H), 9.60 (bs, 1H) | | |
| 126.8 | 1-[4-[4-[[5-chloro-4-(7- | 506 | 1.23 (t, 3H), 2.06 (s, | 36 | 3-(2,5-dichloropyrimidin- |
| | ethylimidazo[1,2-a]pyridin- | | 3H), 2.71 (q, 2H), | | 4-yl)- |
| | 3-yl)pyrimidin- | | 3.14-3.20 (m, 2H), | | 7-ethylimidazo[1, |
| | 2-yl]amino]-3-methoxyphenyl]piperazin- | | 3.20-3.26 (m, 2H), 3.58-3.66 (m, | | 2-a]pyridine |
| | 1-yl]ethanone | | 4H), 3.76 (s, 3H), | | |
| | | | 6.67 (dd, 1H), 6.74 (bs, 1H), | | |
| | | | 7.75 (d, 1H), 7.28 (d, | | |
| | | | 1H), 7.53 (s, 1H), | | |
| | | | 8.43 (s, 1H), 8.67 (s, 1H), | | |
| | | | 8.78 (s, 1H), 9.41 (bs, | | |
| | | | 1H) | | |
| 126.9 | 1-[4-[4-[[5-chloro-4-(7- | 496 | 2.06 (s, 3H), | 11 | 3-(2,5-dichloropyrimidin- |
| | fluoroimidazo[1,2-a]pyridin- | | 3.10-3.19 (m, 2H), 3.19-3.27 (m, | | 4-yl)- |
| | 3-yl)pyrimidin- | | 2H), 3.55-3.66 (m, 4H), | | 7-fluoroimidazo[1, |
| | 2-yl]amino]-3-methoxyphenyl]piperazin- | | 3.78 (s, 3H), 6.56 (dd, | | 2-a]pyridine |
| | 1-yl]ethanone | | 1H), 6.75 (d, 1H), | | |
| | | | 6.93 (bs, 1H), 7.30 (d, 1H), | | |
| | | | 7.66 (dd, 1H), 8.46 (s, | | |
| | | | 1H), 8.71 (s, 1H), | | |
| | | | 8.84 (s, 1H), 9.64 (bs, 1H) | | |
| 126.10 | 1-[4-[4-[[5-chloro-4-(7- | 492 | 2.09 (s, 3H), 2.41 (s, | 44 | 3-(2,5-dichloropyrimidin- |
| | methylimidazo[1,2-a]pyridin- | | 3H), 3.13-3.20 (m, 2H), | | 4-yl)- |
| | 3-yl)pyrimidin- | | 3.20-3.26 (m, 2H), | | 7-methylimidazo[1, |
| | 2-yl]amino]-3-methoxyphenyl]piperazin- | | 3.57-3.66 (m, 4H), | | 2-a]pyridine |
| | 1-yl]ethanone | | 3.76 (s, 3H), 6.56 (dd, 1H), | | |
| | | | 6.72 (bs, 1H), 6.74 (d, | | |
| | | | 1H), 7.29 (d, 1H), | | |
| | | | 7.54 (s, 1H), 8.42 (s, 1H), | | |
| | | | 8.67 (s, 1H), 8.77 (s, | | |
| | | | 1H), 9.43 (bs, 1H) | | |
| 126.11 | 1-[4-[4-[[5-chloro-4-(7- | 508 | 2.06 (s, 3H), | 19 | 3-(2,5-dichloropyrimidin- |
| | methoxyimidazo[1,2-a]pyridin- | | 3.13-3.18 (m, 2H), 3.18-3.24 (m, | | 4-yl)- |
| | 3-yl)pyrimidin- | | 2H), 3.58-3.65 (m, 4H), | | 7- |
| | 2-yl]amino]-3-methoxyphenyl]piperazin- | | 3.76 (s, 3H), 3.89 (s, | | methoxyimidazo[1, |
| | 1-yl]ethanone | | 3H), 6.52 (bs, 1H), | | 2-a]pyridine |
| | | | 6.56 (dd, 1H), 6.75 (dd, 1H), | | |
| | | | 7.15 (d, 1H), 7.28 (d, | | |
| | | | 1H), 8.39 (s, 1H), | | |
| | | | 8.63 (s, 1H), 8.75 (s, 1H), | | |
| | | | 9.42 (s, 1H) | | |
| 126.12 | 1-[4-[4-[[5-chloro-4-[8- | 508 | 2.06 (s, 3H), | 43 | [3-(2,5-dichloropyrimidin- |
| | (hydroxymethyl)imidazo[1, | | 3.12-3.18 (m, 2H), 3.18-3.24 (m, | | 4-yl)imidazo[1, |
| | 2-a]pyridin-3-yl]pyrimidin- | | 2H), 3.58-3.66 (m, 4H), | | 2-a]pyridin- |
| | 2-yl]amino]- | | 3.76 (s, 3H), 4.89 (d, | | 8-yl]methanol |
| | 3- | | 2H), 4.54 (t, 1H), | | |
| | methoxyphenyl]piperazin- | | 6.55 (dd, 1H), 6.73 (d, 1H), | | |
| | 1-yl]ethanone | | 6.91 (bs, 1H), 7.30 (d, | | |
| | | | 1H), 7.49 (d, 1H), | | |
| | | | 8.45 (s, 1H), 8.67 (s, 1H), | | |
| | | | 8.80 (s, 1H), 9.43 (bs, | | |
| | | | 1H) | | |
| 126.13 | 3-[2-[[4-(4-acetylpiperazin- | 503 | 2.06 (s, 3H), | 33 | 3-(2,5-dichloropyrimidin- |
| | 1-yl)-2- | | 3.12-3.18 (m, 2H), 3.18-3.24 (m, | | 4-yl)imidazo |
| | methoxyphenyl]amino]- | | 2H), 3.58-3.65 (m, 4H), | | [1,2-a]pyridine- |
| | 5-chloropyrimidin-4-yl]imidazo[1, | | 3.77 (s, 3H), 6.56 (dd, | | 8- |
| | 2-a]pyridine- | | 1H), 6.74 (d, 1H), | | carbonitrile |
| | 8-carbonitrile | | 7.05 (bs, 1H), 7.30 (d, 1H), | | |
| | | | 8.18 (d, 1H), 8.52 (s, | | |
| | | | 1H), 8.80 (s, 1H), | | |
| | | | 8.93 (s, 1H), 9.78 (bs, 1H) | | |
| 126.14 | 1-[4-[4-[[5-chloro-4-(8- | 496 | 2.06 (s, 3H), | 48 | 3-(2,5-dichloropyrimidin- |
| | fluoroimidazo[1,2-a]pyridin- | | 3.11-3.18 (m, 2H), 3.18-3.25 (m, | | 4-yl)- |
| | 3-yl)pyrimidin- | | 2H), 3.56-3.66 (m, 4H), | | 8-fluoroimidazo[1, |
| | 2-yl]amino]-3-methoxyphenyl]piperazin- | | 3.76 (s, 3H), 6.55 (dd, | | 2-a]pyridine |
| | 1-yl]ethanone | | 1H), 6.74 (d, 1H), | | |
| | | | 6.86 (bs, 1H), 7.29 (d, 1H), | | |
| | | | 7.41 (dd, 1H), 8.50 (s, | | |
| | | | 1H), 8.71 (s, 1H), | | |
| | | | 8.87 (s, 1H), 9.35 (bs, 1H) | | |
| 126.15 | 1-[4-[4-[[5-chloro-4-(8- | 492 | 2.06 (s, 3H), 2.55 (s, | 57 | 3-(2,5-dichloropyrimidin- |
| | methylimidazo[1,2-a]pyridin- | | 3H), 3.11-3.18 (m, 2H), | | 4-yl)- |
| | 3-yl)pyrimidin- | | 3.18-3.24 (m, 2H), | | 8-methylimidazo[1, |
| | 2-yl]amino]-3-methoxyphenyl]piperazin- | | 3.57-3.66 (m, 4H), | | 2-a]pyridine |
| | 1-yl]ethanone | | 3.76 (s, 3H), 6.55 (dd, 1H), | | |
| | | | 6.73 (d, 1H), 6.80 (bs, | | |
| | | | 1H), 7.26-7.34 (m, 2H), | | |
| | | | 8.45 (s, 1H), 8.68 (s, | | |
| | | | 1H), 8.79 (s, 1H), | | |
| | | | 9.40 (bs, 1H) | | |
| 126.16 | 1-[4-[4-[[5-chloro-4-(8- | 508 | 2.07 (s, 3H), | 45 | 3-(2,5-dichloropyrimidin- |
| | methoxyimidazo[1,2-a]pyridin- | | 3.12-3.18 (m, 2H), 3.18-3.24 (m, | | 4-yl)- |
| | 3-yl)pyrimidin- | | 2H), 3.58-3.65 (m, 4H), | | 8- |
| | 2-yl]amino]-3-methoxyphenyl]piperazin- | | 3.76 (s, 3H), 3.96 (s, | | methoxyimidazo[1, |
| | 1-yl]ethanone | | 3H), 6.55 (dd, 1H), | | 2-a]pyridine |
| | | | 6.74 (d, 1H), 6.77 (bs, 1H), | | |
| | | | 6.92 (d, 1H), 7.29 (d, | | |
| | | | 1H), 8.46 (s, 1H), | | |
| | | | 8.60 (s, 1H), 8.79 (s, 1H), | | |
| | | | 9.11 (bs, 1H) | | |
| 126.17 | 1-[4-[4-[[4-(8- | 493 | 2.06 (s, 3H), | 39 | 3-(2,5- |
| | aminoimidazo[1,2- | | 3.12-3.17 (m, 2H), 3.17-3.23 (m, | | dichloropyrimidin- |
| | a]pyridin-3-yl)-5-chloro- | | 2H), 3.58-3.65 (m, 4H), | | 4- |
| | pyrimidin-2-yl]amino]- | | 3.76 (s, 3H), 5.77 (bs, | | yl)imidazo[1,2- |
| | 3-methoxy- | | 2H), 6.49 (d, 1H), | | a]pyridin-8- |
| | phenyl]piperazin-1- | | 6.54 (dd, 1H), 6.62 (bs, 1H), | | amine |
| | yl]ethanone | | 6.72 (d, 1H), 7.29 (d, | | |
| | | | 1H), 8.42 (s, 1H), | | |
| | | | 8.57 (s, 1H), 8.70 (s, 1H), | | |
| | | | 8.82 (bs, 1H) | | |
| 126.18 | 1-[4-[4-[[5-chloro-4-(7- | 512 | 2.07 (s, 3H), | 58 | 7-chloro-3-(2,5- |
| | chloroimidazo[1,2- | | 3.16-3.21 (m, 2H), 3.21-3.26 (m, | | dichloropyrimidin- |
| | a]pyridin-3- | | 2H), 3.59-3.67 (m, 4H), | | 4- |
| | yl)pyrimidin-2- | | 3.78 (s, 3H), 6.58 (dd, | | yl)imidazo[1,2- |
| | yl]amino]-3-methoxy- | | 1H), 6.77 (d, 1H), | | a]pyridine |
| | phenyl]piperazin-1- | | 6.93 (bs, 1H), 7.29 (d, 1H), | | |
| | yl]ethanone | | 7.96 (d, 1H), 8.48 (s, | | |
| | | | 1H), 8.73 (s, 1H), | | |
| | | | 8.87 (s, 1H), 9.51 (bs, 1H) | | |
| 126.19 | 1-[4-[4-[[4-(7- | 556 | (CDCl3) 2.16 (s, 3H), | 57 | 7-bromo-3-(2,5- |
| | bromoimidazo[1,2- | | 3.12-3.23 (m, 4H), | | dichloropyrimidin- |
| | a]pyridin-3-yl)-5-chloro- | | 3.64-3.70 (m, 2H), | | 4- |
| | pyrimidin-2-yl]amino]- | | 3.79-3.86 (m, 2H), | | yl)imidazo[1,2- |
| | 3-methoxy- | | 3.90 (s, 3H), 6.54 (dd, 1H), | | a]pyridine |
| | phenyl]piperazin-1- | | 6.59 (d, 1H), 6.93 (d, | | |
| | yl]ethanone | | 1H), 7.35 (s, 1H), | | |
| | | | 7.93 (s, 1H), 7.97 (d, 1H), | | |
| | | | 8.43 (s, 1H), 8.75 (s, | | |
| | | | 1H), 8.49 (d, 1H) | | |

### Example 127

### 1-[4-[4-[[5-chloro-4-(6-fluoroimidazo[1,2-a]pyridin-3-yl)pyrimidin-2-yl]amino]-3-methoxy-phenyl]piperazin-1-yl]ethanone

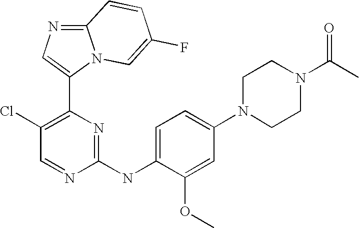

Sodium hydride (19 mg, 0.79 mmol) was added to a stirred solution of N-(4-(4-acetylpiperazin-1-yl)-2-methoxyphenyl)formamide (see Method 41, 88 mg, 0.32 mmol) dissolved in THF (5 mL) at 25° C. under nitrogen. The resulting suspension was stirred at 25° C. for 30 minutes then 3-(2,5-dichloropyrimidin-4-yl)-6-fluoroimidazo[1,2-a]pyridine (see Method 34, 90 mg, 0.32 mmol) was added and the reaction mixture was stirred at 60° C. for 1 hour. The volatiles were evaporated and the residue was triturated in a saturated aqueous solution of ammonium chloride and filtered. The solid was dissolved in DMF and purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford 1-(4-(4-(5-chloro-4-(6-fluoroimidazo[1,2-a]pyridin-3-yl)pyrimidin-2-ylamino)-3-methoxyphenyl)piperazin-1-yl)ethanone (88 mg, 55%) as a yellow solid; NMR spectrum: 2.06 (s, 3H), 3.08-3.16 (m, 2H), 3.16-3.22 (m, 2H), 3.54-3.66 (m, 4H), 3.76 (s, 3H), 6.54 (dd, 1H), 6.71 (d, 1H), 7.27 (d, 1H), 7.59 (ddd, 1H), 7.82 (d, 1H), 8.47 (s, 1H), 8.78 (s, 1H), 8.93 (bs, 1H), 9.61 (bs, 1H); Mass spectrum: 496 (MH+).

### Example 128

### 1-[4-[4-[(5-chloro-4-pyrazolo[1,5-a]pyridin-3-yl-pyrimidin-2-yl)amino]-3-methoxy-phenyl]piperazin-1-yl]ethanone

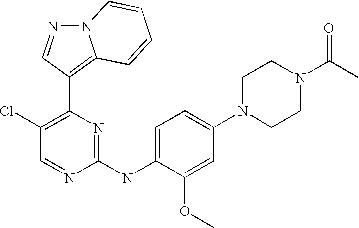

A mixture of 3-(2,5-dichloropyrimidin-4-yl)pyrazolo[1,5-a]pyridine (Method 35, 120 mg, 0.45 mmol), 1-(4-(4-amino-3-methoxyphenyl)piperazin-1-yl)ethanone (Method 7, 113 mg, 0.45 mmol) and p-toluenesulfonic acid hydrate (86 mg, 0.45 mmol) in 2-pentanol (40 mL) was heated at 140° C. during 6 hours. Triethylamine (0.063 mL, 0.45 mmol) was then added, followed by acetic anhydride (0.50 mL, 4.53 mmol) and the reaction mixture was stirred at 25° C. for 3 hours. After evaporation, the crude material was partitioned between dichloromethane and 1N aqueous sodium hydroxide. The organic phase was dried and evaporated and the residue purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford 1-(4-(4-(5-chloro-4-(pyrazolo[1,5-a]pyridin-3-yl)pyrimidin-2-ylamino)-3-methoxyphenyl)piperazin-1-yl)ethanone (107 mg, 49%) as a yellow solid; NMR spectrum: 2.07 (s, 3H), 3.12-3.17 (m, 2H), 3.17-3.23 (m, 2H), 3.58-3.65 (m, 4H), 3.75 (s, 3H), 6.56 (dd, 1H), 6.76 (d, 1H), 7.13 (ddd, 1H), 7.28-7.34 (m, 1H), 7.35 (d, 1H), 8.33 (bs, 1H), 8.35 (s, 1H), 8.54 (s, 1H), 8.82 (d, 1H), 8.94 (s, 1H); Mass spectrum: 478 (MH+).

### Example 129

### 5-chloro-N-(2-methoxy-4-piperazin-1-yl-phenyl)-4-pyrazolo[1,5-a]pyridin-3-yl-pyrimidin-2-amine

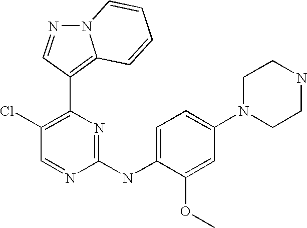

A mixture of 3-(2,5-dichloropyrimidin-4-yl)pyrazolo[1,5-a]pyridine (Method 35, 1.54 g, 5.70 mmol), 1-(4-(4-amino-3-methoxyphenyl)piperazin-1-yl)ethanone (Method 7, 1.42 g, 5.70 mmol) and 4-methylbenzenesulfonic acid hydrate (2.38 g, 12.5 mmol) was heated in 2-pentanol (30 mL) at 140° C. for 20 hours. The reaction mixture was partitioned between saturated aqueous sodium bicarbonate and ethyl acetate and the organic layer was washed with brine, dried over MgSO4, filtrated and concentrated. The crude product was purified by flash chromatography on silica gel eluting with 0 to 8% MeOH(NH3-7N) in methylene chloride to afford the product as an orange foam. Trituration in ethanol/ether and filtration provided 905 mg (73% yield) of the desired compound; NMR spectrum: 2.42 (bs, 1H), 2.82-2.93 (m, 4H), 3.05-3.14 (m, 4H), 3.74 (s, 3H), 6.52 (dd, 1H), 6.66 (d, 1H), 7.12 (ddd, 1H), 7.28 (bs, 1H), 7.30 (d, 1H), 8.30 (bs, 1H), 8.34 (s, 1H), 8.51 (s, 1H), 8.82 (d, 1H), 8.94 (s, 1H); Mass spectrum: 436 (MH+).

### Example 130

### 1-[4-[4-[(5-chloro-4-pyrazolo[1,5-a]pyridin-3-yl-pyrimidin-2-yl)amino]-3-methoxy-phenyl]piperazin-1-yl]-2-hydroxy-ethanone

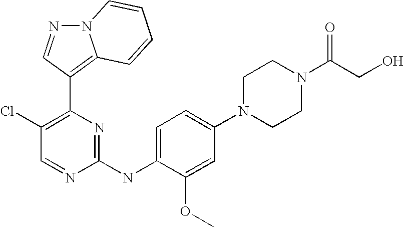

A mixture of 5-chloro-N-(2-methoxy-4-(piperazin-1-yl)phenyl)-4-(pyrazolo[1,5-a]pyridin-3-yl)pyrimidin-2-amine (Example 129, 100 mg, 0.23 mmol), 2-hydroxyacetic acid (14 ul, 0.23 mmol), N-ethyl-N-isopropylpropan-2-amine (0.080 ml, 0.46 mmol) and 2-(3H-[1,2,3]triazolo[4,5-b]pyridin-3-yl)-1,1,3,3-tetramethylisouronium hexafluorophosphate (V) (HATU, 113 mg, 0.30 mmol) in methylene chloride (1 ml) and DMA (0.5 ml) was stirred at 25° C. The reaction mixture being heterogeneous, 1 ml of DMA was added then HATU (50 mg) and 2-hydroxyacetic acid (8 ul) were added to reach complete conversion as shown by LCMS. The reaction mixture was purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent to afford 1-(4-(4-(5-chloro-4-(pyrazolo[1,5-a]pyridin-3-yl)pyrimidin-2-ylamino)-3-methoxyphenyl)piperazin-1-yl)-2-hydroxyethanone (23 mg, 20%) as a yellow foam. NMR spectrum: 3.13-3.25 (m, 4H), 3.48-3.58 (m, 4H), 3.59-3.68 (m, 2H), 3.76 (s, 3H), 4.16 (d, 2H), 4.65 (t, 1H), 6.56 (dd, 1H), 6.73 (d, 1H, 7.12 (ddd, 1H), 7.31 (dd, 1H), 7.35 (d, 1H), 7.44 (ddd, 1H), 8.32 (bs, 1H), 8.35 (s, 1H), 8.54 (s, 1H), 8.83 (d, 1H), 8.94 (s, 1H); Mass spectrum: 494 (MH+).

### Example 131

### (2R)-1-[4-[4-[(5-chloro-4-pyrazolo[1,5-a]pyridin-3-yl-pyrimidin-2-yl)amino]-3-methoxy-phenyl]piperazin-1-yl]-2-hydroxy-propan-1-one

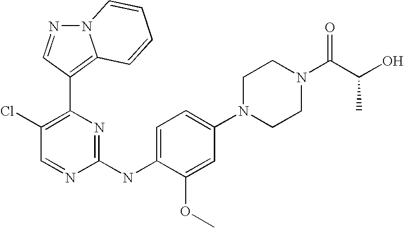

The title compound was prepared in 60% yield from 5-chloro-N-(2-methoxy-4-(piperazin-1-yl)phenyl)-4-(pyrazolo[1,5-a]pyridin-3-yl)pyrimidin-2-amine (Example 129, 120 mg, 0.26 mmol) using the procedure described in example 25.4; NMR spectrum: 1.23 (d, 3H), 3.10-3.26 (m, 4H), 3.56-3.65 (m, 1H), 3.66-3.76 (m, 3H), 3.76 (s, 3H), 4.50 (bs, 1H), 4.99 (bs, 1H), 6.56 (dd, 1H), 6.73 (d, 1H), 7.12 (dd, 1H), 7.31 (dd, 1H), 7.36 (d, 1H), 8.33 (bs, 1H), 8.36 (s, 1H), 8.54 (s, 1H), 8.83 (d, 1H), 8.94 (s, 1H); Mass spectrum: 508 (MH+).

### Example 132

### (2S)-2-amino-1-[4-[4-[(5-chloro-4-pyrazolo[1,5-a]pyridin-3-yl-pyrimidin-2-yl)amino]-3-methoxy-phenyl]piperazin-1-yl]propan-1-one

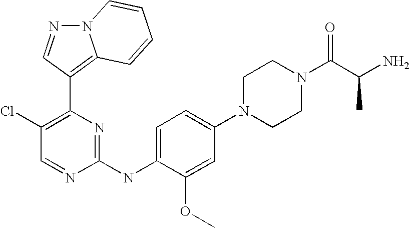

The title compound was prepared in 42% yield from 5-chloro-N-(2-methoxy-4-(piperazin-1-yl)phenyl)-4-(pyrazolo[1,5-a]pyridin-3-yl)pyrimidin-2-amine (Example 129, 120 mg, 0.26 mmol) using the procedure described in example 24.5; NMR spectrum: 1.41 (s, 3H), 1.74 (bs, 2H), 3.10-3.24 (m, 4H), 3.58-3.73 (m, 4H), 3.76 (s, 3H), 3.81 (q, 1H), 6.56 (d, 1H), 6.74 (s, 1H), 7.11 (dd, 1H), 7.31 (dd, 1H), 7.36 (d, 1H), 8.32 (bs, 1H), 8.36 (s, 1H), 8.54 (s, 1H), 8.83 (d, 1H), 8.95 (s, 1H); Mass spectrum: 507 (MH+).

### Example 133

### 1-[4-[4-[(5-chloro-4-pyrazolo[1,5-a]pyridin-3-yl-pyrimidin-2-yl)amino]-3-methoxy-phenyl]piperazin-1-yl]-2-methylamino-ethanone

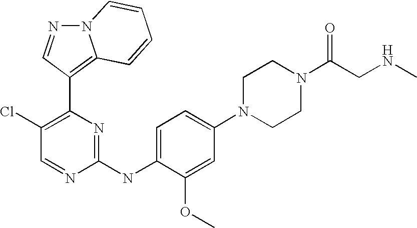

The title compound was prepared in 45% yield from 5-chloro-N-(2-methoxy-4-(piperazin-1-yl)phenyl)-4-(pyrazolo[1,5-a]pyridin-3-yl)pyrimidin-2-amine (Example 129, 120 mg, 0.26 mmol) using the procedure described in example 24.10; NMR spectrum: 1.98 (bs, 1H), 2.29 (s, 3H), 3.12-3.25 (m, 4H), 3.38 (s, 2H), 3.55-3.63 (m, 2H), 3.63-3.70 (m, 2H), 3.75 (s, 3H), 6.56 (dd, 1H), 6.73 (d, 1H), 7.13 (ddd, 1H), 7.13 (dd, 1H), 7.35 (d, 1H), 8.32 (bs, 1H), 8.35 (s, 1H), 8.54 (s, 1H), 8.83 (d, 1H), 8.94 (s, 1H); Mass spectrum: 507 (MH+).

### Example 134

### 2-[4-[4-[(5-chloro-4-pyrazolo[1,5-a]pyridin-3-yl-pyrimidin-2-yl)amino]-3-methoxy-phenyl]piperazin-1-yl]ethanol

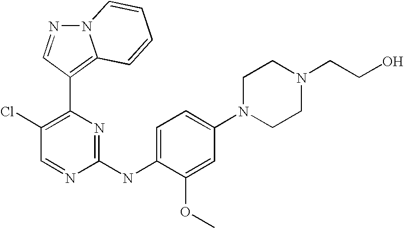

A mixture of 3-(2,5-dichloropyrimidin-4-yl)pyrazolo[1,5-a]pyridine (Method 35, 200 mg, 0.75 mmol), 2-(4-(4-amino-3-methoxyphenyl)piperazin-1-yl)ethanol (Method 7, 190 mg, 0.75 mmol), cesium carbonate (295 mg, 0.91 mmol), palladium (II) acetate (13.5 mg, 0.06 mmol) and (R)-(−)-1-[(S)-2-(dicyclohexylphosphino)ferrocenyl]ethyl-di-t-butylphosphine (41 mg, 0.08 mmol) in DME (5 mL) was degassed with nitrogen, placed into a sealed microwave tube and heated to 140° C. for 30 minutes in a microwave reactor. The reaction mixture was filtered and the filtrate was evaporated. The crude product was purified by flash chromatography on silica gel eluting with 0 to 5% methanol in methylene chloride to afford the title compound as a gum. This gum was purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford 2-(4-(4-(5-chloro-4-(pyrazolo[1,5-a]pyridin-3-yl)pyrimidin-2-ylamino)-3-methoxyphenyl)piperazin-1-yl)ethanol (40 mg, 11%) as a yellow foam. NMR spectrum: 2.46 (t, 2H), 2.56-2.63 (m, 4H), 3.15-3.21 (m, 4H), 3.3-3.59 (m, 2H), 3.74 (s, 3H), 4.45 (t, 1H), 6.52 (dd, 1H), 6.68 (d, 1H), 7.12 (dd, 1H), 7.29 (bs, 1H), 7.31 (d, 1H), 8.30 (bs, 1H), 8.34 (s, 1H), 8.52 (s, 1H), 8.82 (d, 1H), 8.94 (s, 1H); Mass spectrum: 480 (MH+).

### Example 135A

### (2S)-1-[3-[4-[(5-chloro-4-pyrazolo[1,5-a]pyridin-3-yl-pyrimidin-2-yl)amino]-3-methoxy-phenoxy]azetidin-1-yl]-2-hydroxy-propan-1-one

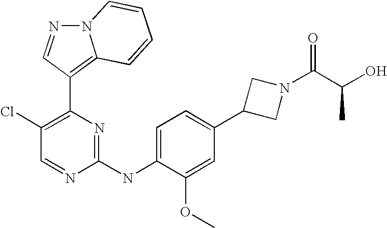

Example 135A was prepared from N-[4-(azetidin-3-yl)-2-methoxy-phenyl]-5-chloro-4-pyrazolo[1,5-a]pyridin-3-yl-pyrimidin-2-amine (90 mg, 0.21 mmol) and (S)-2-hydroxypropanoic acid in 49% yield using the procedure described in example 27.1. The starting N-[4-(azetidin-3-yl)-2-methoxy-phenyl]-5-chloro-4-pyrazolo[1,5-a]pyridin-3-yl-pyrimidin-2-amine was prepared from 3-(2,5-dichloropyrimidin-4-yl)pyrazolo[1,5-a]pyridine (Method 35) according to example 26; NMR spectrum: 2.46 (t, 2H), 2.56-2.63 (m, 4H), 3.15-3.21 (m, 4H), 3.3-3.59 (m, 2H), 3.74 (s, 3H), 4.45 (t, 1H), 6.52 (dd, 1H), 6.68 (d, 1H), 7.12 (dd, 1H), 7.29 (bs, 1H), 7.31 (d, 1H), 8.30 (bs, 1H), 8.34 (s, 1H), 8.52 (s, 1H), 8.82 (d, 1H), 8.94 (s, 1H); Mass spectrum: 479 (MH+).

### Example 135B

### (2R)-1-[3-[4-[(5-chloro-4-pyrazolo[1,5-a]pyridin-3-yl-pyrimidin-2-yl)amino]-3-methoxy-phenoxy]azetidin-1-yl]-2-hydroxy-propan-1-one

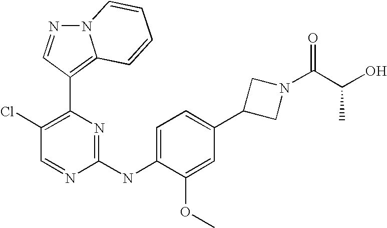

Example 135B was prepared according to example 135A using (R)-2-hydroxypropanoic acid instead of (S)-2-hydroxypropanoic acid; NMR spectrum: 1.21 (d, 3H), 3.76 (s, 3H), 3.81-3.88 (m, 1H), 4.12-4.20 (m, 1H), 4.20-4.27 (m, 1H), 4.37 (ddd, 1H), 4.75 (ddd, 1H), 5.07-5.14 (m, 1H), 5.18 (dd, 1H), 6.44 (dd, 1H), 6.65 (dd, 1H), 7.11 (ddd, 1H), 7.33 (dd, 1H), 7.42 (d, 1H), 8.33 (bs, 1H), 8.37 (s, 1H), 8.63 (s, 1H), 8.84 (d, 1H), 8.94 (s, 1H); Mass spectrum: 479 (MH+).

### Example 136

### 5-chloro-N-[2-methoxy-4-(4-piperidyl)phenyl]-4-pyrazolo[1,5-a]pyridin-3-yl-pyrimidin-2-amine

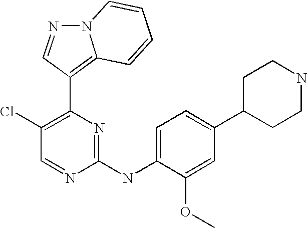

The title compound was prepared in 36% yield from 3-(2,5-dichloropyrimidin-4-yl)pyrazolo[1,5-a]pyridine (Method 35, 218 mg, 0.82 mmol) using the procedure described in example 4; Mass spectrum: 435 (MH+).

### Example 137 to 139

The following compounds were prepared using the procedure as indicated as the “reference example” in the following table. The carboxylic acid used was as recited in the relevant reference example and 5-chloro-4-imidazo[1,2-a]pyridin-3-yl-N-[2-methoxy-4-(4-piperidyl)phenyl]pyrimidin-2-amine in the reference examples was replaced by 5-chloro-N-[2-methoxy-4-(4-piperidyl)phenyl]-4-pyrazolo[1,5-a]pyridin-3-yl-pyrimidin-2-amine (example 136).

| | | | | % | Reference |
|---|---|---|---|---|---|
| Ex. No. | Name | MH+ | NMR spectrum | Yield | example |
| 137 | 1-[4-[4-[(5-chloro-4- | 493 | (DMSOd6 + AcOd4) | 70% | 28.5 |
| | pyrazolo[1,5- | | 1.49-1.61 (m, 1H), 1.61-1.72 (m, | | |
| | a]pyridin-3-yl- | | 1H), 1.83 (bs, 2H), | | |
| | pyrimidin-2- | | 2.62-2.75 (m, 1H), 2.75-2.95 (m, | | |
| | yl)amino]-3-methoxy- | | 1H), 3.01-3.1 (m, 1H), | | |
| | phenyl]-1-piperidyl]- | | 3.77 (bs, 1H), 3.79 (s, 3H), | | |
| | 2-hydroxy-ethanone | | 4.10 (d, 1H), 4.17 (d, 1H), | | |
| | (AZ13110197) | | 4.50-4.60 (m, 1H), 6.83 (d, 1H), | | |
| | | | 6.97 (s, 1H), 7.09 (dd, 1H), | | |
| | | | 7.32 (dd, 1H), 7.63 (d, 1H), | | |
| | | | 8.38 (s, 1H), 8.40 (bs, 1H), | | |
| | | | 8.78 (d, 1H), 8.93 (s, 1H) | | |
| 138 | (2R)-1-[4-[4-[(5- | 507 | 1.21 (d, 1.5H), 1.24 (d, | 50% | 28.2 |
| | chloro-4-pyrazolo[1,5- | | 1.5H), 1.50-1.74 (m, 2H), | | |
| | a]pyridin-3-yl- | | 1.82-1.93 (m, 2H), | | |
| | pyrimidin-2- | | 2.66-2.75 (m, 1H), 2.80-2.90 (m, | | |
| | yl)amino]-3-methoxy- | | 1H), 3.07-3.18 (m, 1H), | | |
| | phenyl]-1-piperidyl]- | | 3.79 (s, 3H), 4.08-4.21 (m, 1H), | | |
| | 2-hydroxy-propan-1- | | 4.44-4.53 (m, 1H), | | |
| | one (AZ13211474) | | 4.53-4.62 (m, 1H), 4.81 (d, 0.5H), | | |
| | | | 4.88 (d, 0.5H), 6.85 (d, 1H), | | |
| | | | 7.00 (s, 1H), 7.14 (ddd, 1H), | | |
| | | | 7.35 (dd, 1H), 7.56 (s, 1H), | | |
| | | | 8.39 (bs, 1H), 8.41 (s, 1H), | | |
| | | | 9.62 (s, 1H), 8.85 (d, 1H), | | |
| | | | 8.96 (s, 1H) | | |
| 139 | (2S)-1-[4-[4-[(5- | 507 | 1.21 (d, 1.5H), 1.24 (d, | 46% | 28.4 |
| | chloro-4-pyrazolo[1,5- | | 1.5H), 1.50-1.74 (m, 2H), | | |
| | a]pyridin-3-yl- | | 1.82-1.93 (m, 2H), | | |
| | pyrimidin-2- | | 2.66-2.75 (m, 1H), 2.80-2.90 (m, | | |
| | yl)amino]-3-methoxy- | | 1H), 3.07-3.18 (m, 1H), | | |
| | phenyl]-1-piperidyl]- | | 3.79 (s, 3H), 4.08-4.21 (m, 1H), | | |
| | 2-hydroxy-propan-1- | | 4.44-4.53 (m, 1H), | | |
| | one | | 4.53-4.62 (m, 1H), 4.81 (d, 0.5H), | | |
| | | | 4.88 (d, 0.5H), 6.85 (d, 1H), | | |
| | | | 7.00 (s, 1H), 7.14 (ddd, 1H), | | |
| | | | 7.35 (dd, 1H), 7.56 (s, 1H), | | |
| | | | 8.39 (bs, 1H), 8.41 (s, 1H), | | |
| | | | 9.62 (s, 1H), 8.85 (d, 1H), | | |
| | | | 8.96 (s, 1H) | | |

### Example 140

### 1-[4-[4-[(5-chloro-4-imidazo[1,2-a]pyridin-3-yl-pyrimidin-2-yl)amino]-3-methoxy-phenyl]-1-piperidyl]-2-(methylamino)ethanone

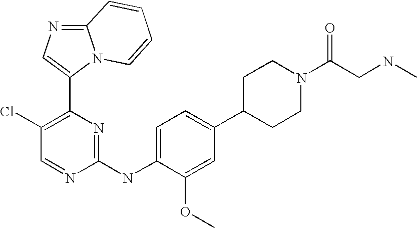

2-(3H-[1,2,3]triazolo[4,5-b]pyridin-3-yl)-1,1,3,3-tetramethylisouronium hexafluorophosphate(V) (165 mg, 0.43 mmol) was added to a stirred solution of 5-chloro-4-(imidazo[1,2-a]pyridin-3-yl)-N-(2-methoxy-4-(piperidin-4-yl)phenyl)pyrimidin-2-amine (Example 4, 145 mg, 0.33 mmol), 2-(tert-butoxycarbonyl(methyl)amino)acetic acid (76 mg, 0.40 mmol) and N-ethyl-N-isopropylpropan-2-amine (0.087 mL, 0.50 mmol) dissolved in DMF (1.5 mL) at 25° C. under nitrogen. The resulting solution was stirred at 25° C. for 30 minutes. The reaction mixture was quenched with water and extracted with methylene chloride. The organic phase was dried over magnesium sulfate and filtered to afford the crude protected product in solution (5 mL) to which TFA (1 mL) was added. After 15 minutes, the volatiles were evaporated. The residue was purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford 1-(4-(4-(5-chloro-4-(imidazo[1,2-a]pyridin-3-yl)pyrimidin-2-ylamino)-3-methoxyphenyl)piperidin-1-yl)-2-(methylamino)ethanone (107 mg, 63%) as a pale yellow foam; NMR spectrum: 1.48-1.60 (m, 1H), 1.60-1.71 (m, 1H), 1.79-1.88 (m, 2H), 2.30 (s, 3H), 2.62-2.70 (m, 1H), 2.77-2.86 (m, 1H), 3.04-3.13 (m, 1H), 3.34 (d partially hidden by H2O, 1H), 3.39 (d, 1H), 3.79 (s, 3H), 3.91-3.99 (m, 1H), 4.53-4.63 (m, 1H), 6.85 (dd, 1H), 6.92 (dd, 1H), 7.02 (s, 1H), 7.48 (d, 1H), 7.51 (ddd, 1H), 7.77 (d, 1H), 8.50 (s, 1H), 8.73 (s, 1H), 8.89 (s, 1H), 9.60 (bs, 1H); Mass spectrum: 506 (MH+).

### Example 141

### (2R)-2-amino-1-[4-[4-[(5-chloro-4-imidazo[1,2-a]pyridin-3-yl-pyrimidin-2-yl)amino]-3-methoxy-phenyl]-1-piperidyl]propan-1-one

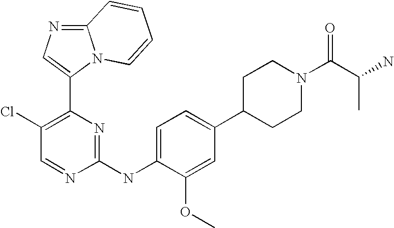

The title compound was prepared according to example 140 using (R)-2-(tert-butoxycarbonylamino)propanoic acid instead of 2-(tert-butoxycarbonyl(methyl)amino)acetic acid; NMR spectrum: 1.08 (d, 1.5H), 1.13 (d, 1.5H), 1.46-1.99 (m, 7H), 2.58-2.72 (m, 1H), 2.78-2.88 (m, 1H), 3.06-3.18 (m, 1H), 3.75-3.84 (m, 1H), 3.80 (s, 3H), 4.01-4.13 (m, 1H), 4.55-4.65 (m, 1H), 6.86 (d, 1H), 6.92 (dd, 1H), 7.02 (s, 1H), 7.46-7.54 (m, 2H), 7.76 (d, 1H), 8.50 (s, 1H), 8.73 (s, 1H), 8.89 (s, 1H), 9.60 (bs, 1H); Mass spectrum: 506 (MH+).

### Example 142

### (2S)-2-amino-1-[4-[4-[(5-chloro-4-imidazo[1,2-a]pyridin-3-yl-pyrimidin-2-yl)amino]-3-methoxy-phenyl]-1-piperidyl]propan-1-one

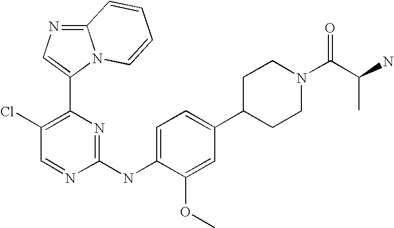

The title compound was prepared according to example 140 using (S)-2-(tert-butoxycarbonylamino)propanoic acid instead of 2-(tert-butoxycarbonyl(methyl)amino)acetic acid; NMR spectrum: 1.08 (d, 1.5H), 1.13 (d, 1.5H), 1.48-1.94 (m, 7H), 2.58-2.72 (m, 1H), 2.78-2.88 (m, 1H), 3.06-3.18 (m, 1H), 3.75-3.84 (m, 1H), 3.80 (s, 3H), 4.01-4.13 (m, 1H), 4.55-4.65 (m, 1H), 6.86 (d, 1H), 6.92 (dd, 1H), 7.02 (s, 1H), 7.46-7.54 (m, 2H), 7.76 (d, 1H), 8.50 (s, 1H), 8.73 (s, 1H), 8.89 (s, 1H), 9.60 (bs, 1H); Mass spectrum: 506 (MH+).

### Example 143

### 5-chloro-4-imidazo[1,2-a]pyridin-3-yl-N-[2-methoxy-4-(9-oxa-3,7-diazabicyclo[3.3.1]nonan-7-yl)phenyl]pyrimidin-2-amine

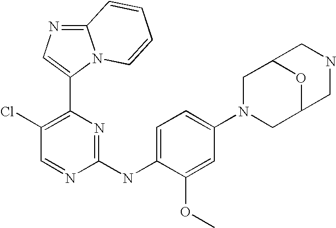

A mixture of 3-(2,5-dichloropyrimidin-4-yl)imidazo[1,2-a]pyridine (Method 1, 120 mg, 0.43 mmol), 4-(9-oxa-3,7-diazabicyclo[3.3.1]nonan-3-yl)-2-methoxyaniline (Method 42, 107 mg, 0.43 mmol) and p-toluenesulfonic acid hydrate (123 mg, 0.65 mmol) in 2-pentanol (1.5 mL) was heated at 140° C. for 2 hours in a microwave reactor. A saturated aqueous solution of sodium bicarbonate was added to the reaction mixture which was then extracted with methylene chloride. The organic extract was washed with brine, dried (MgSO4) and evaporated to dryness. The crude product was purified by flash chromatography on silica gel eluting with 0 to 100% 7N methanolic NH3/EtOAc. A second purification by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent afforded N-(4-(9-oxa-3,7-diazabicyclo[3.3.1]nonan-3-yl)-2-methoxyphenyl)-5-chloro-4-(imidazo[1,2-a]pyridin-3-yl)pyrimidin-2-amine (38 mg, 18%) as a solid; NMR spectrum: 3.01-3.17 (m, 6H), 3.78 (s, 3H), 3.78-3.84 (m, 4H), 6.57 (dd, 1H), 6.76 (s, 1H), 6.89 (bs, 1H), 7.30 (d, 1H), 7.49 (dd, 1H), 7.75 (d, 1H), 8.45 (s, 1H), 8.73 (s, 1H), 8.82 (s, 1H), 9.55 (bs, 1H); Mass spectrum: (MH+).

### Example 144

### 2-[7-[4-[(5-chloro-4-imidazo[1,2-a]pyridin-3-yl-pyrimidin-2-yl)amino]-3-methoxy-phenyl]-9-oxa-3,7-diazabicyclo[3.3.1]nonan-3-yl]ethanol

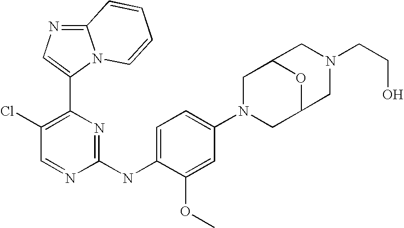

The title compound was prepared according to example 143 using 2-(7-(4-amino-3-methoxyphenyl)-9-oxa-3,7-diazabicyclo[3.3.1]nonan-3-yl)ethanol (Method 43) instead of 4-(9-oxa-3,7-diazabicyclo[3.3.1]nonan-3-yl)-2-methoxyaniline; NMR spectrum: 2.27-2.39 (m, 2H), 2.90-3.03 (m, 2H), 3.07-3.15 (m, 2H), 3.32 (bs partially hidden by H2O, 2H), 3.42-3.52 (m, 2H), 3.69 (d, 2H), 3.74 (s, 3H), 4.06 (bs, 2H), 4.20 (bs, 1H), 6.42 (d, 1H), 6.53 (s, 1H), 6.84 (bs, 1H), 7.21 (d, 1H), 7.47 (dd, 1H), 7.75 (d, 1H), 8.43 (s, 1H), 8.73 (s, 1H), 8.77 (s, 1H), 9.50 (bs, 1H); Mass spectrum: (MH+).

### Example 145

### 1-[4-[3-[(5-chloro-4-imidazo[1,2-a]pyridin-3-yl-pyrimidin-2-yl)amino]-4-methoxy-phenyl]piperazin-1-yl]-2-(methylamino)ethanone

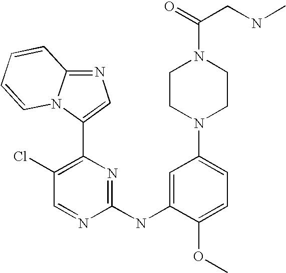

The title compound was prepared according to example 140 using 5-chloro-4-imidazo[1,2-a]pyridin-3-yl-N-(2-methoxy-5-piperazin-1-yl-phenyl)pyrimidin-2-amine (Example 3.10) instead of 5-chloro-4-(imidazo[1,2-a]pyridin-3-yl)-N-(2-methoxy-4-(piperidin-4-yl)phenyl)pyrimidin-2-amine; NMR spectrum: 2.28 (s, 3H), 2.90-3.04 (m, 4H), 3.32 (s, 2H), 3.46-3.53 (m, 2H), 3.53-3.60 (m, 2H), 3.76 (s, 3H), 6.80 (dd, 1H), 6.96 (t, 1H), 7.03 (d, 1H), 7.34 (s, 1H), 7.50 (dd, 1H), 7.77 (d, 1H), 8.54 (s, 1H), 8.71 (s, 1H), 8.86 (s, 1H), 9.56 (bs, 1H); Mass spectrum: (MH+).

### Example 146

### 1-[4-[3-[(5-chloro-4-imidazo[1,2-a]pyridin-3-yl-pyrimidin-2-yl)amino]-4-methoxy-phenyl]piperazin-1-yl]-2-(dimethylamino)ethanone

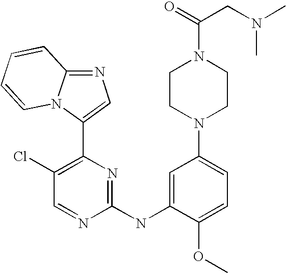

2-(3H-[1,2,3]triazolo[4,5-b]pyridin-3-yl)-1,1,3,3-tetramethylisouronium hexafluorophosphate(V) (100 mg, 0.26 mmol) was added portionwise to a stirred solution of 5-chloro-4-(imidazo[1,2-a]pyridin-3-yl)-N-(2-methoxy-5-(piperazin-1-yl)phenyl)pyrimidin-2-amine (Example 3.10, 88 mg, 0.20 mmol), 2-(dimethylamino)acetic acid (23 mg, 0.22 mmol) and N-ethyl-N-isopropylpropan-2-amine (0.053 mL, 0.30 mmol) in DMF (1 mL) at room temperature. The resulting solution was stirred at room temperature for 1.5 hours. This mixture was then purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford 1-(4-(3-(5-chloro-4-(imidazo[1,2-a]pyridin-3-yl)pyrimidin-2-ylamino)-4-methoxyphenyl)piperazin-1-yl)-2-(dimethylamino)ethanone (69 mg, 66%) as a yellow solid; NMR spectrum: 2.17 (s, 6H), 2.91-3.03 (m, 4H), 3.08 (s, 2H), 3.49-3.58 (m, 2H), 3.58-3.66 (m, 2H), 3.75 (s, 3H), 6.80 (dd, 1H), 6.95 (t, 1H), 7.03 (d, 1H), 7.33 (d, 1H), 7.50 (ddd, 1H), 7.77 (d, 1H), 8.54 (s, 1H), 8.71 (s, 1H), 8.95 (s, 1H), 9.56 (bs, 1H); Mass spectrum: (MH+).

### Examples 147 to 152

The following compounds were prepared according to the procedure as indicated as the “reference example” in the following table using 3-(2,5-dichloropyrimidin-4-yl)pyrazolo[1,5-a]pyridine (Method 35) instead of 3-(2,5-dichloropyrimidin-4-yl)imidazo[1,2-a]pyridine as a starting material.

| Example | | | | Reference |
|---|---|---|---|---|
| Number | Name | MH+ | NMR spectrum | example |
| 147 | 5-chloro-N-(2- | 436 | 2.72-2.80 (m, 4H), 2.86-2.94 (m, 4H), | 3.10 |
| | methoxy-5-piperazin- | | 3.73 (s, 3H), 6.71 (dd, 1H), 6.97 (d, 1H), | |
| | 1-yl-phenyl)-4- | | 7.13 (ddd, 1H), 7.38 (dd, 1H), 7.40 (d, | |
| | pyrazolo[1,5- | | 1H), 8.37 (d, 1H), 8.44 (s, 1H), 8.52 (s, | |
| | a]pyridin-3-yl- | | 1H), 8.85 (d, 1H), 8.93 (s, 1H) | |
| | pyrimidin-2-amine | | | |
| 148 | 2-[4-[(5-chloro-4- | 426 | 2.81-2.91 (m, 1H), 3.62-3.69 (m, 2H), | 70 |
| | pyrazolo[1,5- | | 3.72-3.78 (m, 2H), 3.78 (s, 3H), 4.60 (t, | |
| | a]pyridin-3-yl- | | 2H), 6.84 (d, 1H), 6.98 (s, 1H), 7.12 (dd, | |
| | pyrimidin-2- | | 1H), 7.42 (dd, 1H), 7.50 (d, 1H), | |
| | yl)amino]-3-methoxy- | | 8.39 (bs, 1H), 8.40 (s, 1H), 8.63 (s, 1H), | |
| | phenyl]propane-1,3- | | 8.84 (d, 1H), 8.97 (s, 1H) | |
| | diol | | | |
| 149 | 5-chloro-N-[2- | 478 | 2.30 (m, 1H), 3.00-3.08 (m, 2H), | 143 |
| | methoxy-4-(9-oxa-3,7- | | 3.08-3.17 (m, 4H), 3.77 (s, 3H), | |
| | diazabicyclo[3.3.1]nonan- | | 3.77-3.84 (m, 4H), 6.57 (dd, 1H), 6.75 (d, 1H), | |
| | 7-yl)phenyl]-4- | | 7.12 (dd, 1H), 7.32 (bs, 1H), 7.35 (d, | |
| | pyrazolo[1,5- | | 1H), 8.33 (bs, 1H), 8.35 (s, 1H), | |
| | a]pyridin-3-yl- | | 8.546 (s, 1H), 8.83 (d, 1H), 8.95 (s, 1H) | |
| | pyrimidin-2-amine | | | |
| 150 | 2-[7-[4-[(5-chloro-4- | 522 | 2.33 (t, 2H), 2.45-2.53 (m partially | 144 |
| | pyrazolo[1,5- | | hidden by DMSOd5, 2H), 2.97 (d, 2H), | |
| | a]pyridin-3-yl- | | 3.10 (dd, 2H), 3.44-3.50 (m, 2H), | |
| | pyrimidin-2- | | 3.67 (d, 2H), 3.73 (s, 3H), 4.03-4.08 (m, 2H), | |
| | yl)amino]-3-methoxy- | | 4.21 (t, 1H), 6.41 (dd, 1H), 6.51 (d, 1H), | |
| | phenyl]-9-oxa-3,7- | | 7.09 (dd, 1H), 7.24 (d, 1H), 7.27 (bs, | |
| | diazabicyclo[3.3.1]nonan- | | 1H), 8.22 (bs, 1H), 8.32 (s, 1H), 8.51 (s, | |
| | 3-yl]ethanol | | 1H), 8.81 (d, 1H), 8.94 (s, 1H) | |
| 151 | 1-[4-[3-[(5-chloro-4- | 507 | 1.95 (bs, 1H), 2.26 (s, 3H), | 145 |
| | pyrazolo[1,5- | | 2.91-3.03 (m, 4H), 3.31 (s, 2H), 3.45-3.51 (m, | |
| | a]pyridin-3-yl- | | 2H), 3.51-3.60 (m, 2H), 3.75 (s, 3H), | |
| | pyrimidin-2- | | 6.67 (dd, 1H), 7.00 (d, 1H), 7.13 (ddd, | |
| | yl)amino]-4-methoxy- | | 1H), 7.38 (ddd, 1H), 7.46 (d, 1H), | |
| | phenyl]piperazin-1- | | 8.38 (d, 1H), 8.45 (s, 1H), 8.55 (s, 1H), | |
| | yl]-2- | | 8.85 (d, 1H), 8.94 (s, 1H) | |
| | (methylamino)ethanone | | | |
| 152 | 1-[4-[3-[(5-chloro-4- | 521 | 2.16 (s, 6H), 2.90-3.03 (m, 4H), 3.07 (s, | 146 |
| | pyrazolo[1,5- | | 2H), 3.49-3.55 (m, 2H), 3.58-3.65 (m, | |
| | a]pyridin-3-yl- | | 2H), 3.75 (s, 3H), 6.77 (dd, 1H), 7.00 (d, | |
| | pyrimidin-2- | | 1H), 7.13 (ddd, 1H), 7.38 (dd, 1H), | |
| | yl)amino]-4-methoxy- | | 7.45 (d, 1H), 8.38 (d, 1H), 8.45 (s, 1H), | |
| | phenyl]piperazin-1- | | 8.55 (s, 1H), 8.85 (d, 1H), 8.94 (s, 1H) | |
| | yl]-2- | | | |
| | (dimethylamino)ethanone | | | |

### Example 153

### N-[3-[(5-chloro-4-pyrazolo[1,5-a]pyridin-3-yl-pyrimidin-2-yl)amino]-4-methoxy-phenyl]-2-(dimethylamino)acetamide

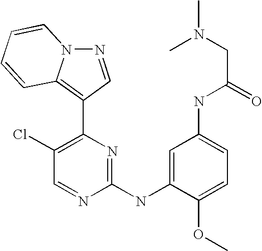

The title compound was prepared according to example 146 using N3-(5-chloro-4-pyrazolo[1,5-a]pyridin-3-yl-pyrimidin-2-yl)-4-methoxy-benzene-1,3-diamine instead of 5-chloro-4-(imidazo[1,2-a]pyridin-3-yl)-N-(2-methoxy-5-(piperazin-1-yl)phenyl)pyrimidin-2-amine; NMR spectrum: 2.94 (s, 6H), 3.11 (s, 2H), 3.78 (s, 3H), 7.06 (s, 1H), 7.13 (ddd, 1H), 7.35 (ddd, 1H), 7.51 (dd, 1H), 7.95 (d, 1H), 8.40 (d, 1H), 8.45 (s, 1H), 8.69 (d, 1H), 8.85 (d, 1H), 8.97 (s, 1H), 9.65 (s, 1H); Mass spectrum: (MH+).

The starting N3-(5-chloro-4-pyrazolo[1,5-a]pyridin-3-yl-pyrimidin-2-yl)-4-methoxy-benzene-1,3-diamine was prepared as follows:

A mixture of 3-(2,5-dichloropyrimidin-4-yl)pyrazolo[1,5-a]pyridine (Method 35, 205 mg, 0.73 mmol), N-(3-amino-4-methoxyphenyl)acetamide (132 mg, 0.73 mmol) and 4-methylbenzenesulfonic acid hydrate (140 mg, 0.73 mmol) in NMP (2 mL) was stirred at 150° C. for 2.5 hours. The reaction mixture was diluted with methylene chloride and poured into an aqueous K2CO3 solution. The mixture was extracted with methylene chloride the organic layer was dried over MgSO4 and concentrated to dryness. Purification of the crude product by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent afforded N-(3-(5-chloro-4-(pyrazolo[1,5-a]pyridin-3-yl)pyrimidin-2-ylamino)-4-methoxyphenyl)acetamide (111 mg, 37%) as a beige solid; NMR spectrum: 2.00 (s, 3H), 3.77 (s, 3H), 7.04 (d, 1H), 7.14 (ddd, 1H), 7.34 (ddd, 1H), 7.45 (dd, 1H), 7.87 (d, 1H), 8.41 (d, 1H), 8.44 (s, 1H), 8.69 (s, 1H), 8.95 (d, 1H), 8.97 (s, 1H), 9.80 (s, 1H).

A mixture of N-(3-(5-chloro-4-(pyrazolo[1,5-a]pyridin-3-yl)pyrimidin-2-ylamino)-4-methoxyphenyl)acetamide (110 mg, 0.27 mmol) and conc HCl (0.52 mL) in water (1 mL) was stirred at 80° C. for 3 hours. The reaction mixture was diluted with water and basified with NaOH 2N. The resulting precipitate was filtered, washed with water and dried under high vacuum with P2O5 at 50° C. overnight to afford N1-(5-chloro-4-(pyrazolo[1,5-a]pyridin-3-yl)pyrimidin-2-yl)-6-methoxybenzene-1,3-diamine (100 mg, 100%) as a beige solid; NMR spectrum: 3.68 (s, 3H), 4.68 (bs, 2H), 6.41 (dd, 1H), 6.82 (d, 1H), 7.03 (d, 1H), 7.14 (dd, 1H), 7.41 (dd, 1H), 8.42 (s, 1H), 8.45 (s, 1H), 8.48 (d, 1H), 8.85 (d, 1H), 8.96 (s, 1H).

### Example 154

### 1-[6-[(5-chloro-4-pyrazolo[1,5-a]pyridin-3-yl-pyrimidin-2-yl)amino]-5-methoxy-indolin-1-yl]ethanone

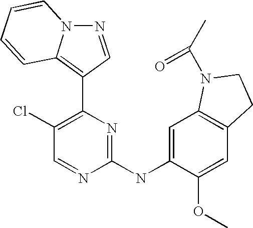

1-(6-amino-5-methoxyindolin-1-yl)ethanone (Described in Bioorganic & Medicinal Chemistry Letters (2009), p. 360-364, 50 mg, 0.24 mmol), 3-(2,5-dichloropyrimidin-4-yl)pyrazolo[1,5-a]pyridine (Method 35, 53 mg, 0.19 mmol) and hydrogen chloride 4M in dioxane (0.280 mL, 1.12 mmol) were suspended in 2,2,2-trifluoroethanol (2 mL) and sealed into a microwave tube. The reaction mixture was heated at 130° C. for 45 minutes in the microwave reactor. The mixture was diluted with methylene chloride and poured into a K2CO3 aqueous solution. Following extraction with methylene chloride and evaporation, the crude product was purified by flash chromatography on silica gel eluting with 0 to 4% ethanol in methylene chloride to afford 1-(6-(5-chloro-4-(pyrazolo[1,5-a]pyridin-3-yl)pyrimidin-2-ylamino)-5-methoxyindolin-1-yl)ethanone (30 mg, 37%) as a yellow solid; NMR spectrum: 2.12 (s, 3H), 3.20 (t, 2H), 3.72 (s, 3H), 4.14 (t, 2H), 7.05 (s, 1H), 7.10 (ddd, 1H), 7.30 (dd, 1H), 8.25 (s, 1H), 8.35 (d, 1H), 8.38 (s, 1H), 8.71 (s, 1H), 8.82 (d, 1H), 8.95 (s, 1H); Mass spectrum: 435 (MH+).

### Example 155

### N-(5-chloro-4-pyrazolo[1,5-a]pyridin-3-yl-pyrimidin-2-yl)-5-methoxy-indolin-6-amine

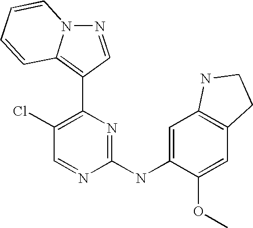

Hydrochloric acid (1.66 mL) was added dropwise to a stirred solution of 1-(6-(5-chloro-4-(pyrazolo[1,5-a]pyridin-3-yl)pyrimidin-2-ylamino)-5-methoxyindolin-1-yl)ethanone (Example 154, 217 mg, 0.50 mmol) in methanol (2 mL). The resulting solution was heated at 100° C. for 1 hour in a sealed microwave tube. The mixture was evaporated under reduce pressure. The pH was adjusted to 10 with a solution of sodium hydroxide 6N. The mixture was extracted with methylene chloride, dried over MgSO4, filtered and evaporated under reduce pressure. The crude product was purified by flash chromatography on silica gel eluting with 1 to 3% methanolic ammonia (7 N) in methylene chloride to afford N-(5-chloro-4-(pyrazolo[1,5-a]pyridin-3-yl)pyrimidin-2-yl)-5-methoxyindolin-6-amine (165 mg, 84%) as a yellow solid; NMR spectrum: CDCl3: 3.03 (t, 2H), 3.55 (t, 2H), 3.86 (s, 3H), 6.81 (s, 1H), 6.86 (ddd, 1H), 7.37 (ddd, 1H), 7.63 (s, 1H), 7.85 (s, 1H), 8.36 (s, 1H), 8.57 (d, 1H), 8.61 (d, 1H), 8.92 (s, 1H); Mass spectrum: 393 (MH+).

### Example 156

### 1-[6-[(5-chloro-4-pyrazolo[1,5-a]pyridin-3-yl-pyrimidin-2-yl)amino]-5-methoxy-indolin-1-yl]-2-(methylamino)ethanone

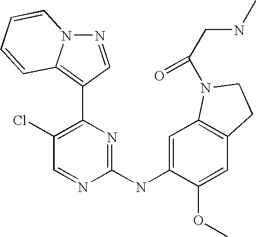

The title compound was prepared according to example 140 using N-(5-chloro-4-pyrazolo[1,5-a]pyridin-3-yl-pyrimidin-2-yl)-5-methoxy-indolin-6-amine (Example 155) instead of 5-chloro-4-(imidazo[1,2-a]pyridin-3-yl)-N-(2-methoxy-4-(piperidin-4-yl)phenyl)pyrimidin-2-amine; NMR spectrum: 2.29 (s, 3H), 3.21 (t, 2H), 3.41 (s, 2H), 3.74 (s, 3H), 4.12 (t, 2H), 7.07 (s, 1H), 7.10 (dd, 1H), 7.31 (dd, 1H), 8.32 (s, 1H), 8.38 (d, 1H), 8.40 (s, 1H), 8.73 (s, 1H), 8.83 (d, 1H), 8.96 (s, 1H); Mass spectrum: 464 (MH+).

### Example 157

### 1-[6-[(5-chloro-4-pyrazolo[1,5-a]pyridin-3-yl-pyrimidin-2-yl)amino]-5-methoxy-indolin-1-yl]-2-(dimethylamino)ethanone

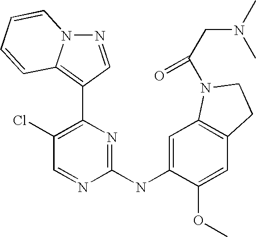

The title compound was prepared according to example 146 using N-(5-chloro-4-pyrazolo[1,5-a]pyridin-3-yl-pyrimidin-2-yl)-5-methoxy-indolin-6-amine (Example 155) instead of 5-chloro-4-(imidazo[1,2-a]pyridin-3-yl)-N-(2-methoxy-5-(piperazin-1-yl)phenyl)pyrimidin-2-amine; NMR spectrum: 2.22 (s, 6H), 3.17 (t, 2H), 3.20 (s, 2H), 3.74 (s, 3H), 4.22 (t, 2H), 7.07 (s, 1H), 7.10 (dd, 1H), 7.30 (dd, 1H), 8.29 (s, 1H), 8.34 (d, 1H), 8.40 (s, 1H), 8.72 (s, 1H), 8.83 (d, 1H), 8.96 (s, 1H); Mass spectrum: 478 (MH+).

### Examples 158 to 169

The following compounds were prepared according to the procedure as indicated as the “reference example” in the following table using 3-(2-chloro-5-methyl-pyrimidin-4-yl)pyrazolo[1,5-a]pyridine (Method 44) instead of 3-(2,5-dichloropyrimidin-4-yl)pyrazolo[1,5-a]pyridine as a starting material.

| | | | | Reference |
|---|---|---|---|---|
| Ex. No. | Name | MH+ | NMR spectrum | example |
| 158 | 1-[4-[3-methoxy-4- | 458 | 2.06 (s, 3H), 2.38 (s, 3H), 3.06-3.13 (m, | 128 |
| | [(5-methyl-4- | | 2H), 3.14-3.20 (m, 2H), 3.55-3.66 (m, | |
| | pyrazolo[1,5- | | 4H), 3.80 (s, 3H), 6.53 (dd, 1H), 6.72 (d, | |
| | a]pyridin-3-yl- | | 1H), 7.07 (ddd, 1H), 7.31 (ddd, 1H), | |
| | pyrimidin-2- | | 7.61 (d, 1H), 7.92 (s, 1H), 8.20 (s, 1H), | |
| | yl)amino]phenyl]piperazin- | | 8.41 (d, 1H), 8.55 (s, 1H), 8.80 (d, 1H) | |
| | 1-yl]ethanone | | | |
| 159 | N-(2-methoxy-4- | 416 | 2.28 (bs, 1H), 2.37 (s, 3H), | 129 |
| | piperazin-1-yl- | | 2.81-2.91 (bs, 4H), 3.01-3.11 (m, 4H), 3.78 (s, | |
| | phenyl)-5-methyl-4- | | 3H), 6.49 (d, 1H), 6.65 (s, 1H), 7.07 (dd, | |
| | pyrazolo[1,5- | | 1H), 7.29 (dd, 1H), 7.56 (d, 1H), 7.90 (s, | |
| | a]pyridin-3-yl- | | 1H), 8.19 (s, 1H), 8.40 (d, 1H), 8.55 (s, | |
| | pyrimidin-2-amine | | 1H), 8.79 (d, 1H) | |
| 160 | 1-[4-[3-methoxy-4- | 487 | 1.96 (bs, 1H), 2.29 (s, 3H), 2.37 (s, 3H), | 133 |
| | [(5-methyl-4- | | 3.09-3.20 (m, 4H), 3.36 (s, 2H), | |
| | pyrazolo[1,5- | | 3.54-3.61 (bs, 2H), 3.61-3.70 (bs, 2H), | |
| | a]pyridin-3-yl- | | 3.80 (s, 3H), 6.53 (dd, 1H), 6.71 (d, 1H), | |
| | pyrimidin-2- | | 7.08 (ddd, 1H), 7.31 (ddd, 1H), 7.62 (d, 1H), | |
| | yl)amino]phenyl]piperazin- | | 7.92 (s, 1H), 8.20 (s, 1H), 8.42 (d, 1H), | |
| | 1-yl]-2- | | 8.55 (s, 1H), 8.80 (d, 1H) | |
| | (methylamino)ethanone | | | |
| 161 | 2-[4-[3-methoxy-4- | 460 | 2.3 (s, 3H), 2.45 (t, 2H), 2.55-2.63 (m, | 134 |
| | [(5-methyl-4- | | 4H), 3.10-3.19 (m, 4H), 3.51-3.59 (m, | |
| | pyrazolo[1,5- | | 2H), 3.79 (s, 3H), 4.45 (t, 1H), 6.50 (d, | |
| | a]pyridin-3-yl- | | 1H), 6.66 (s, 1H), 7.08 (dd, 1H), | |
| | pyrimidin-2- | | 7.30 (dd, 1H), 7.55 (d, 1H), 7.90 (s, 1H), | |
| | yl)amino]phenyl]piperazin- | | 8.19 (s, 1H), 8.40 (d, 1H), 8.55 (s, 1H), | |
| | 1-yl]ethanol | | 8.79 (d, 1H) | |
| 162 | N-[2-methoxy-4-(4- | 415 | 1.53 (dd, 1H), 1.56 (dd, 1H), | 136 |
| | piperidyl)phenyl]-5- | | 1.70-1.76 (m, 2H), 2.40 (s, 3H), 2.53-2.65 (m, | |
| | methyl-4- | | 3H), 3.01-3.08 (m, 2H), 3.83 (s, 3H), | |
| | pyrazolo[1,5- | | 6.80 (dd, 1H), 6.92 (d, 1H), 7.09 (ddd, | |
| | a]pyridin-3-yl- | | 1H), 7.35 (ddd, 1H), 7.85 (d, 1H), | |
| | pyrimidin-2-amine | | 7.98 (s, 1H), 8.25 (s, 1H), 8.46 (d, 1H), | |
| | | | 8.57 (s, 1H), 8.82 (d, 1H) | |
| 163 | 2-hydroxy-1-[4-[3- | 473 | 1.49-1.60 (m, 1H), 1.60-1.73 (m, 1H), | 137 |
| | methoxy-4-[(5- | | 1.77-1.88 (m, 2H), 2.40 (s, 3H), | |
| | methyl-4- | | 2.65-2.74 (m, 1H), 2.74-2.84 (m, 1H), | |
| | pyrazolo[1,5- | | 3.02-3.12 (m, 1H), 3.75-3.83 m, 1H), 3.83 (s, | |
| | a]pyridin-3-yl- | | 3H), 4.11 (dd, 1H), 4.16 (dd, 1H), | |
| | pyrimidin-2- | | 4.48 (t, 1H), 4.50-4.58 (m, 1H), 6.81 (dd, | |
| | yl)amino]phenyl]-1- | | 1H), 6.96 (s, 1H), 7.09 (ddd, 1H), | |
| | piperidyl]ethanone | | 7.36 (ddd, 1H), 7.88 (d, 1H), 7.98 (s, 1H), | |
| | | | 8.26 (s, 1H), 8.47 (d, 1H), 8.57 (s, 1H), | |
| | | | 8.82 (d, 1H) | |
| 164 | (2R)-2-hydroxy-1-[4- | 487 | 1.20 (d, 1.5H), 1.23 (d, 1.5H), | 138 |
| | [3-methoxy-4-[(5- | | 1.44-1.72 (m, 2H), 1.79-1.90 (m, 2H), 2.39 (s, | |
| | methyl-4- | | 3H), 2.64-2.72 (m, 1H), 2.75-2.85 (m, | |
| | pyrazolo[1,5- | | 1H), 3.05-3.15 (m, 1H), 3.84 (s, 3H), | |
| | a]pyridin-3-yl- | | 4.06-4.18 (m, 1H), 4.42-4.60 (m, 2H), | |
| | pyrimidin-2- | | 4.80 (d, 0.5H), 4.86 (d, 0.5H), 6.81 (d, | |
| | yl)amino]phenyl]-1- | | 1H), 6.95 (s, 1H), 7.09 (ddd, 1H), | |
| | piperidyl]propan-1- | | 7.36 (dd, 1H), 7.88 (d, 1H), 7.99 (s, 1H), | |
| | one | | 8.26 (s, 1H), 8.47 (d, 1H), 8.58 (s, 1H), | |
| | | | 8.82 (d, 1H) | |
| 165 | N-(2-methoxy-5- | 416 | 2.17 (bs, 1H), 2.39 (bs, 3H), | 147 |
| | piperazin-1-yl- | | 2.69-2.77 (m, 4H), 2.82-2.88 (m, 4H), 3.79 (s, | |
| | phenyl)-5-methyl-4- | | 3H), 6.56 (dd, 1H), 6.92 (d, 1H), | |
| | pyrazolo[1,5- | | 7.10 (dd, 1H), 7.39 (dd, 1H), 7.82 (d, 1H), | |
| | a]pyridin-3-yl- | | 7.91 (s, 1H), 8.31 (s, 1H), 8.43 (d, 1H), | |
| | pyrimidin-2-amine | | 8.57 (s, 1H), 8.83 (d, 1H) | |
| 166 | 2-[7-[3-methoxy-4- | 502 | (323°K) 2.37 (bs, 2H), 2.39 (s, 3H), | 150 |
| | [(5-methyl-4- | | 2.45-2.53 (m partially hidden by | |
| | pyrazolo[1,5- | | DMSOd5, 2H), 2.97 (bs, 2H), | |
| | a]pyridin-3-yl- | | 3.08-3.17 (m 2H), 3.42-3.47 (m, 2H), | |
| | pyrimidin-2- | | 3.56-3.71 (m, 2H), 3.81 (s, 3H), 4.06 (bs, 2H), | |
| | yl)amino]phenyl]-9- | | 4.21 (t, 1H), 6.42 (bs, 1H), 6.53 (bs, | |
| | oxa-3,7- | | 1H), 7.06 (dd, 1H), 7.30 (dd, 1H), | |
| | diazabicyclo[3.3.1]nonan- | | 7.56 (bs, 1H), 7.75 (s, 1H), 8.20 (s, 1H), | |
| | 3-yl]ethanol | | 8.38 (d, 1H), 8.53 (s, 1H), 8.76 (d, 1H) | |
| 167 | 1-[4-[4-methoxy-3- | 487 | 2.40 (s, 6H), 2.89-3.00 (m, 4H), | 151 |
| | [(5-methyl-4- | | 3.41-3.48 (m, 2H), 3.51-3.57 (m, 2H), | |
| | pyrazolo[1,5- | | 3.64 (s, 2H), 3.80 (s, 3H), 6.07 (bs, 1H), | |
| | a]pyridin-3-yl- | | 6.63 (dd, 1H), 6.96 (d, 1H), 7.10 (ddd, 1H), | |
| | pyrimidin-2- | | 7.41 (dd, 1H), 7.87 (d, 1H), 7.95 (s, 1H), | |
| | yl)amino]phenyl]piperazin- | | 8.32 (s, 1H), 8.44 (d, 1H), 8.59 (d, 1H), | |
| | 1-yl]-2- | | 8.84 (d, 1H) | |
| | (methylamino)ethanone | | | |
| 168 | 1-[5-methoxy-6-[(5- | 415 | 2.12 (s, 3H), 2.38 (s, 3H), 3.16 (t, 2H), | 154 |
| | methyl-4- | | 3.76 (s, 3H), 4.12 (t, 2H), 7.00 (s, 1H), | |
| | pyrazolo[1,5- | | 7.06 (ddd, 1H), 7.28 (dd, 1H), 8.08 (s, | |
| | a]pyridin-3-yl- | | 1H), 8.22 (s, 1H), 8.44 (d, 1H), 8.48 (s, | |
| | pyrimidin-2- | | 1H), 8.56 (s, 1H), 8.78 (d, 1H) | |
| | yl)amino]indolin-1- | | | |
| | yl]ethanone | | | |
| 169 | 2-(dimethylamino)-1- | 458 | 2.26 (s, 6H), 2.39 (s, 3H), 3.17 (t, 2H), | 157 |
| | [5-methoxy-6-[(5- | | 3.23 (s, 2H), 3.77 (s, 3H), 4.19 (t, 2H), | |
| | methyl-4- | | 7.02 (s, 1H), 7.04 (dd, 1H), 7.27 (dd, | |
| | pyrazolo[1,5- | | 1H), 8.09 (s, 1H), 8.24 (s, 1H), 8.44 (d, | |
| | a]pyridin-3-yl- | | 1H), 8.53 (s, 1H), 8.57 (s, 1H), 8.78 (s, | |
| | pyrimidin-2- | | 1H) | |
| | yl)amino]indolin-1- | | | |
| | yl]ethanone | | | |

### Examples 170 to 176

The following compounds were prepared according to the procedure as indicated as the “reference example” in the following table using 3-(2-chloro-5-fluoro-pyrimidin-4-yl)pyrazolo[1,5-a]pyridine (Method 45) instead of 3-(2,5-dichloropyrimidin-4-yl)pyrazolo[1,5-a]pyridine as a starting material.

| | | | | Reference |
|---|---|---|---|---|
| Ex. No. | Name | MH+ | NMR spectrum | example |
| 170 | 1-[4-[4-[(5-fluoro-4- | 462 | 2.07 (s, 3H), 3.09-3.16 (m, 2H), | 128 |
| | pyrazolo[1,5- | | 3.16-3.22 (m, 2H), 3.58-3.67 (m, 4H), | |
| | a]pyridin-3-yl- | | 3.77 (s, 3H), 6.57 (dd, 1H), 6.72 (d, | |
| | pyrimidin-2- | | 1H), 7.13 (ddd, 1H), 7.39 (dd, 1H), | |
| | yl)amino]-3-methoxy- | | 7.46 (d, 1H), 8.32 (s, 1H), 8.36 (d, | |
| | phenyl]piperazin-1- | | 1H), 8.45 (d, 1H), 8.55 (d, 1H), | |
| | yl]ethanone | | 8.85 (d, 1H) | |
| 171 | 5-fluoro-N-(2- | 420 | 2.85-2.90 (m, 4H), 3.05-3.11 (m, | 129 |
| | methoxy-4-piperazin- | | 4H), 3.75 (s, 3H), 6.52 (dd, 1H), | |
| | 1-yl-phenyl)-4- | | 6.66 (d, 1H), 7.13 (ddd, 1H), 7.37 (dd, | |
| | pyrazolo[1,5- | | 1H), 7.41 (d, 1H), 8.29 (s, 1H), | |
| | a]pyridin-3-yl- | | 8.35 (d, 1H), 8.42 (d, 1H), 8.55 (d, 1H), | |
| | pyrimidin-2-amine | | 8.85 (d, 1H) | |
| 172 | 1-[4-[4-[(5-fluoro-4- | 491 | 1.93 (bs, 1H), 2.30 (s, 3H), | 133 |
| | pyrazolo[1,5- | | 3.10-3.23 (m, 4H), 3.37 (s, 2H), 3.56-3.53 (bs, | |
| | a]pyridin-3-yl- | | 2H), 3.53-3.71 (bs, 2H), 3.78 (s, 3H), | |
| | pyrimidin-2- | | 6.57 (dd, 1H), 6.72 (d, 1H), | |
| | yl)amino]-3-methoxy- | | 7.13 (ddd, 1H), 7.39 (dd, 1H), 7.47 (d, | |
| | phenyl]piperazin-1- | | 1H), 8.32 (d, 1H), 8.36 (d, 1H), | |
| | yl]-2- | | 8.44 (d, 1H), 8.55 (d, 1H), 8.85 (d, 1H) | |
| | (methylamino)ethanone | | | |
| 173 | 5-fluoro-N-[2- | 419 | 1.54-1.64 (m, 2H), 1.70-1.78 (m, | 136 |
| | methoxy-4-(4- | | 2H), 2.20 (bs, 1H), 2.56-2.64 (m, | |
| | piperidyl)phenyl]-4- | | 2H), 3.01-3.09 (m, 2H), 3.33-3.35 (m | |
| | pyrazolo[1,5- | | partially hidden by H2O, 1H), | |
| | a]pyridin-3-yl- | | 6.84 (dd, 1H), 6.94 (d, 1H), 7.15 (ddd, | |
| | pyrimidin-2-amine | | 1H), 7.41 (dd, 1H), 7.65 (d, 1H), | |
| | | | 8.39 (s, 1H), 8.41 (d, 1H), 8.49 (d, 1H), | |
| | | | 8.57 (d, 1H), 8.86 (d, 1H) | |
| 174 | 5-fluoro-N-(2- | 420 | 2.79 (bs, 4H), 2.92 (bs, 4H), 3.76 (s, | 147 |
| | methoxy-5-piperazin- | | 3H), 6.66 (d, 1H), 6.95 (d, 1H), | |
| | 1-yl-phenyl)-4- | | 7.17 (dd, 1H), 7.46 (dd, 1H), 7.55 (s, 1H), | |
| | pyrazolo[1,5- | | 8.30 (s, 1H), 8.46 (d, 1H), 8.52 (d, | |
| | a]pyridin-3-yl- | | 1H), 8.58 (d, 1H), 8.87 (d, 1H) | |
| | pyrimidin-2-amine | | | |
| 175 | 2-[7-[4-[(5-fluoro-4- | 506 | 2.30-2.37 (m, 2H), 2.50-2.54 (m | 150 |
| | pyrazolo[1,5- | | partially hidden by DMSOd5, 2H), | |
| | a]pyridin-3-yl- | | 2.93-3.03 (m, 2H), 3.30-3.34 (m | |
| | pyrimidin-2- | | partially hidden by H2O, 2H), | |
| | yl)amino]-3-methoxy- | | 3.43-3.51 (m, 2H), 3.64-3.67 (m, 2H), | |
| | phenyl]-9-oxa-3,7- | | 3.75 (s, 3H), 4.02-4.09 (m, 2H), | |
| | diazabicyclo[3.3.1]nonan- | | 4.21 (bs, 1H), 6.42 (dd, 1H), 6.52 (d, 1H), | |
| | 3-yl]ethanol | | 7.12 (ddd, 1H), 7.31-7.39 (m, 2H), | |
| | | | 8.28 (s, 1H), 8.33 (d, 1H), 8.36 (bs, | |
| | | | 1H), 8.54 (d, 1H), 8.84 (d, 1H) | |
| 176 | 2-(dimethylamino)-1- | 462 | 2.24 (s, 6H), 3.17 (t, 2H), 3.19 (s, | 157 |
| | [6-[(5-fluoro-4- | | 2H), 3.77 (s, 3H), 4.20 (t, 2H), | |
| | pyrazolo[1,5- | | 7.02 (s, 1H), 7.10 (ddd, 1H), 7.36 (ddd, | |
| | a]pyridin-3-yl- | | 1H), 8.25 (bs, 1H), 8.39 (d, 1H), | |
| | pyrimidin-2- | | 8.44 (d, 1H), 9.50 (bs, 1H), 8.57 (d, 1H), | |
| | yl)amino]-5-methoxy- | | 8.80 (d, 1H) | |
| | indolin-1-yl]ethanone | | | |

### Examples 177 to 184

The following compounds were prepared according to the procedure as indicated as the “reference example” in the following table using 3-(2,5-dichloropyrimidin-4-yl)-8-fluoroimidazo[1,2-a]pyridine (Method 34) instead of 3-(2,5-dichloropyrimidin-4-yl)imidazo[1,2-a]pyridine as a starting material.

| | | | | Reference |
|---|---|---|---|---|
| Ex. No. | Name | MH+ | NMR spectrum | example |
| 177 | 1-[4-[4-[[5-chloro-4- | 495 | 1.45-1.60 (m, 1H), 1.62-1.73 (m, 1H), | 3.21 |
| | (8-fluoroimidazo[1,2- | | 1.78-1.88 (m, 2H), 2.06 (s, 3H), | |
| | a]pyridin-3- | | 2.56-2.65 (m, 1H), 2.77-2.85 (m, 1H), | |
| | yl)pyrimidin-2- | | 3.11-3.19 (m, 1H), 3.80 (s, 3H), | |
| | yl]amino]-3-methoxy- | | 3.92-4.00 (m, 1H), 4.54-4.61 (m, 1H), 6.86 (d, | |
| | phenyl]-1- | | 1H), 6.85-6.94 (m, 1H), 7.04 (s, 1H), | |
| | piperidyl]ethanone | | 7.44 (dd, 1H), 7.48 (d, 1H), 8.55 (s, 1H), | |
| | | | 8.72 (s, 1H), 8.96 (s, 1H), 9.42 (bs, 1H) | |
| 178 | (2R)-1-[4-[4-[[5- | 526 | 1.16 (d, 3H), 3.04-3.21 (m, 4H), | 25.4 |
| | chloro-4-(8- | | 3.49-3.58 (m, 1H), 3.58-3.70 (m, 3H), | |
| | fluoroimidazo[1,2- | | 3.70 (s, 3H), 4.38-4.48 (m, 1H), 4.93 (d, 1H), | |
| | a]pyridin-3- | | 6.49 (dd, 1H), 6.68 (s, 1H), 6.79 (bs, | |
| | yl)pyrimidin-2- | | 1H), 7.22 (d, 1H), 7.34 (ddd, 1H), | |
| | yl]amino]-3-methoxy- | | 8.43 (s, 1H), 8.65 (s, 1H), 8.81 (s, 1H), | |
| | phenyl]piperazin-1- | | 9.30 (bs, 1H) | |
| | yl]-2-hydroxy-propan- | | | |
| | 1-one | | | |
| 179 | 1-[4-[4-[[5-chloro-4- | 511 | 1.50-1.63 (m, 1H), 1.63-1.74 (m, 1H), | 28.5 |
| | (8-fluoroimidazo[1,2- | | 1.78-1.90 (m, 2H), 2.66-2.77 (m, 1H), | |
| | a]pyridin-3- | | 2.78-2.88 (m, 1H), 3.03-3.13 (m, 1H), | |
| | yl)pyrimidin-2- | | 3.80 (s, 3H), 3.82 (bs, 1H), | |
| | yl]amino]-3-methoxy- | | 4.08-4.23 (m, 2H), 4.49 (t, 1H), 4.52-4.60 (m, | |
| | phenyl]-1-piperidyl]- | | 1H), 6.86 (d, 1H), 6.87-6.94 (m, 1H), | |
| | 2-hydroxy-ethanone | | 7.03 (s, 1H), 7.43 (dd, 1H), 7.48 (d, 1H), | |
| | | | 8.55 (s, 1H), 8.72 (s, 1H), 8.96 (s, 1H), | |
| | | | 9.42 (bs, 1H) | |
| 180 | 1-[4-[4-[[5-chloro-4- | 512 | 3.07-3.19 (m, 4H), 3.41-3.52 (m, 2H), | 40 |
| | (8-fluoroimidazo[1,2- | | 3.52-3.65 (m, 2H), 3.70 (s, 3H), 4.09 (d, | |
| | a]pyridin-3- | | 2H), 4.59 (t, 1H), 6.49 (dd, 1H), 6.67 (d, | |
| | yl)pyrimidin-2- | | 1H), 6.79 (bs, 1H), 7.22 (d, 1H), | |
| | yl]amino]-3-methoxy- | | 7.35 (dd, 1H), 8.43 (s, 1H), 8.64 (s, 1H), | |
| | phenyl]piperazin-1- | | 8.81 (s, 1H), 9.30 (bs, 1H) | |
| | yl]-2-hydroxy- | | | |
| | ethanone | | | |
| 181 | 2-[4-[[5-chloro-4-(8- | 444 | 2.82-2.91 (m, 1H), 3.61-3.69 (m, 2H), | 70 |
| | fluoroimidazo[1,2- | | 3.71-3.77 (m, 2H), 3.78 (s, 3H), 4.61 (t, | |
| | a]pyridin-3- | | 2H), 6.85 (dd, 1H), 6.97 (dd, 1H), | |
| | yl)pyrimidin-2- | | 7.00 (s, 1H), 7.40 (dd, 1H), 7.42 (d, 1H), | |
| | yl]amino]-3-methoxy- | | 8.54 (s, 1H), 8.74 (s, 1H), 8.98 (s, 1H), | |
| | phenyl]propane-1,3- | | 9.41 (bs, 1H) | |
| | diol | | | |
| 182 | 1-[4-[4-[[5-chloro-4- | 524 | 1.49-1.59 (m, 1H), 1.60-1.71 (m, 1H), | 140 |
| | (8-fluoroimidazo[1,2- | | 1.79-1.87 (m, 2H), 2.04 (bs, 1H), | |
| | a]pyridin-3- | | 2.31 (s, 3H), 3.39 (d partailly hidden by | |
| | yl)pyrimidin-2- | | H2O, 2H), 2.60-2.71 (m, 1H), | |
| | yl]amino]-3-methoxy- | | 2.77-2.88 (m, 1H), 3.04-3.15 (m, 1H), 3.80 (s, | |
| | phenyl]-1-piperidyl]- | | 3H), 3.92-4.01 (m, 1H), 4.53-4.63 (m, | |
| | 2- | | 1H), 6.86 (dd, 1H), 6.87-6.95 (m, 1H), | |
| | (methylamino)ethanone | | 7.03 (s, 1H), 7.43 (dd, 1H), 7.48 (d, 1H), | |
| | | | 8.55 (s, 1H), 8.72 (s, 1H), 8.96 (s, 1H), | |
| | | | 9.41 (bs, 1H) | |
| 183 | 5-chloro-4-(8- | 496 | | 143 |
| | fluoroimidazo[1,2- | | | |
| | a]pyridin-3-yl)-N-[2- | | | |
| | methoxy-4-(9-oxa-3,7- | | | |
| | diazabicyclo[3.3.1]nonan- | | | |
| | 7- | | | |
| | yl)phenyl]pyrimidin- | | | |
| | 2-amine | | | |
| 184 | 2-[7-[4-[[5-chloro-4- | 540 | 2.32 (t, 2H), 2.45-2.52 (m partially | 144 |
| | (8-fluoroimidazo[1,2- | | hidden by DMSOd5, 2H), 2.96 (d, 2H), | |
| | a]pyridin-3- | | 3.11 (dd, 2H), 3.42-3.49 (m, 2H), | |
| | yl)pyrimidin-2- | | 3.68 (d, 2H), 3.75 (s, 3H), 4.02-4.09 (m, 2H), | |
| | yl]amino]-3-methoxy- | | 4.19 (t, 1H), 6.41 (dd, 1H), 6.53 (d, 1H), | |
| | phenyl]-9-oxa-3,7- | | 6.80 (bs, 1H), 7.19 (d, 1H), 7.38 (dd, | |
| | diazabicyclo[3.3.1]nonan- | | 1H), 8.47 (s, 1H), 8.73 (s, 1H), 8.83 (s, | |
| | 3-yl]ethanol | | 1H), 9.29 (bs, 1H) | |

### Example 185

### 1-[4-[4-[[5-chloro-4-[7-(dimethylaminomethyl)imidazo[1,2-a]pyridin-3-yl]pyrimidin-2-yl]amino]-3-methoxy-phenyl]piperazin-1-yl]ethanone

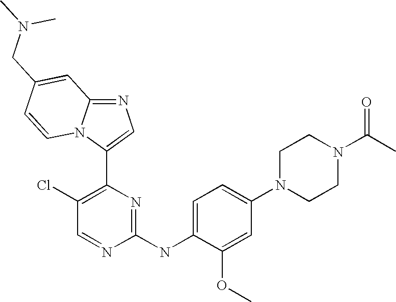

A solution of 1-(4-(4-(5-chloro-4-(7-(hydroxymethyl)imidazo[1,2-a]pyridin-3-yl)pyrimidin-2-ylamino)-3-methoxyphenyl)piperazin-1-yl)ethanone (Example 125, 1.3 g, 2.56 mmol) in methylene chloride (30 mL) and triethylamine (0.43 mL, 3.1 mmol) was treated with methanesulfonyl chloride (0.198 mL, 2.56 mmol) and the reaction mixture was stirred at room temperature for 1 hour. The mixture was then diluted with water (2 ml) and extracted with methylene chloride. The organic layer was evaporated to dryness, triturated in diethyl ether and filtrated to give a solid as a mixture of [3-[2-[4-(4-acetylpiperazin-1-yl)-2-methoxy-anilino]-5-chloro-pyrimidin-4-yl]imidazo[1,2-a]pyridin-7-yl]methyl methanesulfonate and 1-[4-[4-[[5-chloro-4-[7-(chloromethyl)imidazo[1,2-a]pyridin-3-yl]pyrimidin-2-yl]amino]-3-methoxy-phenyl]piperazin-1-yl]ethanone which was used in the next step without further purification. A tenth of this sample was diluted in acetonitrile (1 mL) was treated with N-ethyl-N-isopropylpropan-2-amine (0.17 mL, 0.98 mmol) and dimethylamine 2M in THF (0.5 mL, 1 mmol). The reaction mixture was stirred at room temperature for 20 hours. The solvent was evaporated and the residue was purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent to afford 1-(4-(4-(5-chloro-4-(7-((dimethylamino)methyl)imidazo[1,2-a]pyridin-3-yl)pyrimidin-2-ylamino)-3-methoxyphenyl)piperazin-1-yl)ethanone (65 mg, 49% over 2 steps) as a yellow solid; NMR spectrum: 2.07 (s, 3H), 2.18 (s, 6H), 3.16-3.26 (m, 4H), 3.47 (s, 2H), 3.59-3.68 (m, 4H), 3.74 (s, 3H), 6.57 (dd, 1H), 6.75 (d, 1H), 6.82 (bs, 1H), 7.25 (d, 1H), 7.57 (s, 1H), 8.44 (s, 1H), 8.69 (s, 1H), 8.80 (s, 1H), 9.34 (bs, 1H); Mass spectrum: 535 (MH+).

### Example 186

### 1-[4-[4-[[5-chloro-4-[7-(methylaminomethyl)imidazo[1,2-a]pyridin-3-yl]pyrimidin-2-yl]amino]-3-methoxy-phenyl]piperazin-1-yl]ethanone

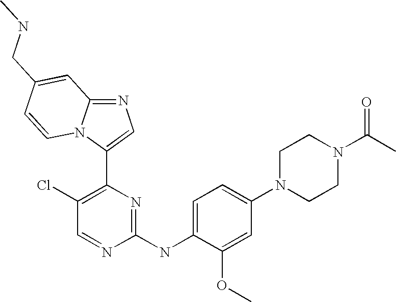

This compound was prepared following the procedure described for Example 185, using methylamine instead of dimethylamine; NMR spectrum: 2.07 (s, 3H), 2.28 (s, 3H), 3.14-3.20 (m, 2H), 3.20-3.27 (m, 2H), 3.56-3.67 (m, 4H), 3.72 (s, 2H), 3.76 (s, 3H), 6.56 (dd, 1H), 6.74 (d, 1H), 6.86 (bs, 1H), 7.27 (d, 1H), 7.61 (s, 1H), 8.43 (s, 1H), 8.68 (s, 1H), 8.79 (s, 1H), 9.42 (s, 1H); Mass spectrum: 521 (MH+).

### Example 187

### 1-[4-[4-[[5-chloro-4-[7-(morpholinomethyl)imidazo[1,2-a]pyridin-3-yl]pyrimidin-2-yl]amino]-3-methoxy-phenyl]piperazin-1-yl]ethanone

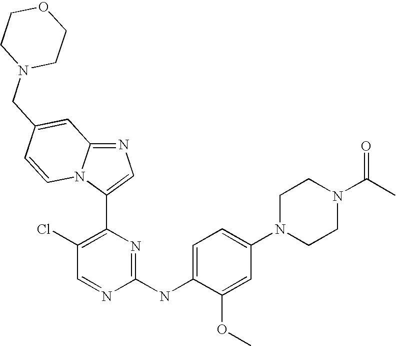

This compound was prepared following the procedure described for Example 185, using morpholine instead of dimethylamine; NMR spectrum: 2.06 (s, 3H), 2.33-2.43 (m, 4H), 3.16-3.22 (m, 2H), 3.22-3.27 (m, 2H), 3.56 (s, 2H), 3.57-3.61 (m, 4H), 3.61-3.68 (m, 4H), 3.75 (s, 3H), 6.57 (dd, 1H), 6.75 (d, 1H), 6.84 (bs, 1H), 7.28 (d, 1H), 7.62 (s, 1H), 8.44 (s, 1H), 8.70 (s, 1H), 8.80 (s, 1H), 9.43 (s, 1H); Mass spectrum: 577 (MH+).

### Example 188

### N-[[3-[2-[4-(4-acetylpiperazin-1-yl)-2-methoxy-anilino]-5-chloro-pyrimidin-4-yl]imidazo[1,2-a]pyridin-7-yl]methyl]-N-methyl-acetamide

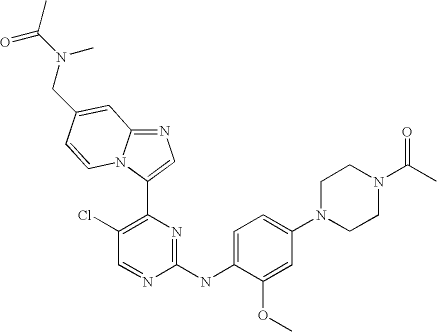

A solution of 1-(4-(4-(5-chloro-4-(7-((methylamino)methyl)imidazo[1,2-a]pyridin-3-yl)pyrimidin-2-ylamino)-3-methoxyphenyl)piperazin-1-yl)ethanone (Example 186, 80 mg, 0.15 mmol) in methylene chloride (1 mL) was treated with acetic anhydride (0.017 mL, 0.15 mmol) and stirred at room temperature for 1 hour. The solvent was evaporated and the residue was purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent to afford N-((3-(2-(4-(4-acetylpiperazin-1-yl)-2-methoxyphenylamino)-5-chloropyrimidin-4-yl)imidazo[1,2-a]pyridin-7-yl)methyl)-N-methylacetamide (20 mg, 23%) as a yellow solid; NMR spectrum: (323° K) 2.06 (s, 3H), 2.07 (s, 0.9H), 2.10 (s, 2.1H), 2.84 (s, 0.9H), 2.97 (s, 2.1H), 3.21 (bs partially hidden by H2O, 4H), 3.63 (bs, 4H), 3.76 (s, 3H), 4.57 (s, 1.4H), 4.66 (s, 0.6H), 6.56 (dd, 1H), 6.64 (d, 0.3H), 6.69 (d, 0.7H), 6.71 (d, 1H), 7.33 (d, 1H), 7.51 (s, 0.3H), 7.54 (s, 0.7H), 8.42 (s, 0.7H), 8.43 (s, 0.3H), 8.61 (s, 0.7H), 8.62 (s, 0.3H), 8.61 (s, 0.7H), 8.62 (s, 0.3H); Mass spectrum: 563 (MH+).

### Example 189

### 5-chloro-N-[2-methoxy-5-(9-oxa-3,7-diazabicyclo[3.3.1]nonan-7-yl)phenyl]-4-yrazolo[1,5-a]pyridin-3-yl-pyrimidin-2-amine

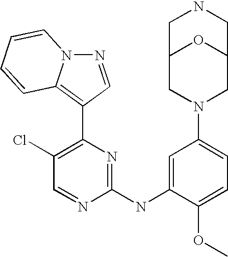

This compound was prepared following the procedure described for Example 129, using 1-[3-(3-amino-4-methoxy-phenyl)-9-oxa-3,7-diazabicyclo[3.3.1]nonan-7-yl]ethanone instead of 1-(4-(4-amino-3-methoxyphenyl)piperazin-1-yl)ethanone; NMR spectrum: 2.24 (bs, 1H), 2.87 (d, 2H), 2.93-3.09 (m, 4H), 3.56 (d, 2H), 3.64 (dd, 1H), 3.74 (s, 3H), 6.80 (dd, 1H), 7.03 (d, 1H), 7.11 (ddd, 1H), 7.35 (dd, 1H), 7.40 (d, 1H), 8.33 (bs, 1H), 8.44 (s, 1H), 8.60 (s, 1H), 8.84 (d, 1H), 8.94 (s, 1H); Mass spectrum: 478 (MH+).

The 1-[3-(3-amino-4-methoxy-phenyl)-9-oxa-3,7-diazabicyclo[3.3.1]nonan-7-yl]ethanone used as the starting material was prepared as follows:

Acetyl chloride (1.15 mL, 16.2 mmol) was added dropwise to a stirred suspension of 3-benzyl-9-oxa-3,7-diazabicyclo[3.3.1]nonane dihydrochloride (WO-2001/028992, 2 g, 6.47 mmol) and cesium carbonate (7.38 g, 22.6 mmol) in methylene chloride (20 mL) at room temperature. The resulting suspension was stirred at room temperature for 3 hours, then filtered, washed with methylene chloride and concentrated to dryness to afford 1-(7-benzyl-9-oxa-3,7-diazabicyclo[3.3.1]nonan-3-yl)ethanone (1.77 g, quantitative) as a pale yellow oil which crystallised on standing. This product was mixed with 10% palladium on charcoal (250 mg) in EtOAc (5 mL) and MeOH (75 mL). This mixture was hydrogenated under 60 psi at room temperature for 16 hours. The resulting suspension was filtered and concentrated to dryness to afford 1-(9-oxa-3,7-diazabicyclo[3.3.1]nonan-3-yl)ethanone (1.17 g, 85% pure) as a pale pink oil which crystallised on standing. This material was used in the next step without further purification; NMR spectrum: 1.99 (s, 3H), 2.77-2.83 (m, 1H), 2.94-3.01 (m, 2H), 3.02-3.10 (m, 2H), 3.50 (ddd, 1H), 3.63-3.69 (m, 2H), 3.77-3.82 (m, 1H), 4.24-4.30 (m, 1H). A mixture of 1-(9-oxa-3,7-diazabicyclo[3.3.1]nonan-3-yl)ethanone (646 mg, 3.79 mmol), 4-bromo-1-methoxy-2-nitrobenzene (800 mg, 3.45 mmol), cesium carbonate (1.46 g, 4.48 mmol) and 1,1 bis(di-tert-butylphosphino)ferrocene palladium dichloride (178 mg, 0.28 mmol) in toluene (8 mL) was degassed with nitrogen and stirred at 90° C. for 72 hours. The reaction mixture was allowed to cool to room temperature, quenched with water (50 mL) and extracted with methylene chloride (2×30 ml). The combined organic extracts were washed with brine, dried over magnesium sulfate and concentrated to dryness. The crude product was purified by flash chromatography on silica gel eluting with 0 to 4% methanol in methylene chloride to afford 1-(7-(4-methoxy-3-nitrophenyl)-9-oxa-3,7-diazabicyclo[3.3.1]nonan-3-yl)ethanone (620 mg, 56%); NMR spectrum: 1.86 (s, 3H), 2.93-3.04 (m, 3H), 3.50-3.60 (m, 2H), 3.83 (s, 3H), 3.83-3.89 (m, 1H), 3.91-3.96 (m, 1H), 3.96-4.02 (m, 2H), 4.40-4.47 (m, 1H), 7.71 (dd, 1H), 7.24 (d, 1H), 7.26 (d, 1H).

A suspension of 1-(7-(4-methoxy-3-nitrophenyl)-9-oxa-3,7-diazabicyclo[3.3.1]nonan-3-yl)ethanone (640 mg, 1.99 mmol) and platinum(IV) oxide (64 mg, 0.28 mmol) in EtOH (30 mL) was hydrogenated under 1.6 bar at room temperature for 2.5 hours. The resulting suspension was filtered, washed with EtOAc and the filtrate was concentrated to dryness. The crude product was purified by flash chromatography on silica gel eluting with 0 to 4% methanol in methylene chloride to afford 1-(7-(3-amino-4-methoxyphenyl)-9-oxa-3,7-diazabicyclo[3.3.1]nonan-3-yl)ethanone (487 mg, 84%) as a foam; NMR spectrum: 1.86 (s, 3H), 2.80-2.92 (m, 2H), 2.94-3.04 (m, 1H), 3.30-3.35 (m partially hidden by H20, 1H), 3.51-3.60 (m, 2H), 3.66 (s, 3H), 3.88-3.96 (m, 3H), 4.38-4.45 (m, 1H), 4.57 (bs, 2H), 6.01 (dd, 1H), 6.21 (d, 1H), 6.64 (d, 1H).

### Example 190

### 2-[[4-[(5-chloro-4-pyrazolo[1,5-a]pyridin-3-yl-pyrimidin-2-yl)amino]-3-methoxy-phenyl]methyl]propane-1,3-diol

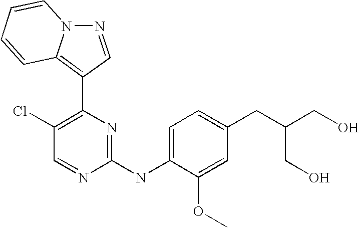

This compound was prepared following the procedure described for Example 129, using 2-[(4-amino-3-methoxy-phenyl)methyl]propane-1,3-diol instead of 1-(4-(4-amino-3-methoxyphenyl)piperazin-1-yl)ethanone; NMR spectrum: 1.79-1.89 (m, 1H), 2.62 (d, 2H), 3.38-3.48 (m, 4H), 3.79 (s, 3H), 4.45 (t, 2H), 6.81 (dd, 1H), 6.95 (s, 1H), 7.13 (dd, 1H), 7.36 (dd, 1H), 7.53 (d, 1H), 8.41 (bs, 1H), 8.42 (s, 1H), 8.62 (s, 1H), 8.86 (d, 1H), 8.97 (s, 1H); Mass spectrum: 440 (MH+).

The 2-[(4-amino-3-methoxy-phenyl)methyl]propane-1,3-diol used as the starting material was prepared as follows:

Sodium hydride (0.440 g, 11.0 mmol) was added to a stirred solution of dimethyl malonate (1.25 ml, 11.0 mmol) in DMF (25 ml) at room temperature. The solution was stirred for one hour then 4-(bromomethyl)-2-methoxy-1-nitrobenzene (J. Org. Chem. 1980, p. 2243, 1.23 g, 5.00 mmol) was added. The reaction mixture was stirred at 25° C. for 2 h30 then concentrated in vacuo. The residue was mixed with a saturated aqueous solution of ammonium chloride and extracted with ethyl acetate. The organic extract was washed with water and brine, dried over magnesium sulfate and concentrated. The crude product was purified by flash chromatography on silica gel eluting with 0 to 30% EtOAc/petroleum ether to afford dimethyl 2-(3-methoxy-4-nitrobenzyl)malonate (1.08 g, 72%) as a pale yellow oil; NMR spectrum: 3.18 (d, 2H), 3.64 (s, 6H), 3.90 (s, 3H), 4.05 (t, 1H), 6.95 (dd, 1H), 7.27 (d, 1H), 7.80 (d, 1H). Borane methyl sulfide complex (0.792 mL, 8.91 mmol) was added in one portion to a stirred solution of dimethyl 2-(3-methoxy-4-nitrobenzyl)malonate (1.06 g, 3.57 mmol) in THF (20 mL) at 25° C. under argon. The resulting solution was stirred at 80° C. for 2 days. After cooling, the reaction mixture was diluted with 10 ml of saturated aqueous NaHCO3 and extracted with EtOAc. The organic extract was dried (MgSO4), filtered and evaporated to dryness. The crude product was purified by flash chromatography on silica gel eluting with 100% EtOAc then 30% MeOH/EtOAc to afford 2-(3-methoxy-4-nitrobenzyl)propane-1,3-diol (100 mg, 11%) as a pale yellow oil.

A solution of 2-(3-methoxy-4-nitrobenzyl)propane-1,3-diol (93 mg, 0.39 mmol) in ethanol (2 ml) was hydrogenated under 1.4 atm at 25° C. for 4 hours in the presence of platinum (IV) oxide (17 mg). The resulting suspension was filtered and the filtrate was concentrated to dryness to afford 2-(4-amino-3-methoxybenzyl)propane-1,3-diol (70 mg, 86%) as an oil; NMR spectrum: 1.63-1.70 (m, 1H), 2.39 (d, 2H), 3.30-3.38 (m, 4H partially hidden by water), 3.73 (s, 3H), 4.31 (t, 2H), 4.44 (s, 2H), 6.47 (d, 1H), 6.52 (d, 1H), 6.60 (d, 1H).

### Example 191

### N-[3-[[5-chloro-4-(8-fluoroimidazo[1,2-a]pyridin-3-yl)pyrimidin-2-yl]amino]-4-methoxy-phenyl]-2-(dimethylamino)acetamide

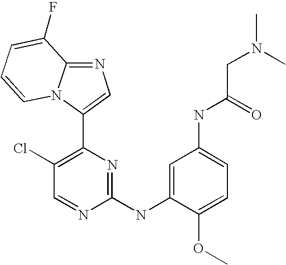

This compound was prepared following the procedure described for Example 153, using 3-(2,5-dichloropyrimidin-4-yl)-8-fluoroimidazo[1,2-a]pyridine (Method 34) instead of 3-(2,5-dichloropyrimidin-4-yl)pyrazolo[1,5-a]pyridine as a starting material; NMR spectrum: 2.24 (s, 6H), 3.02 (s, 2H), 3.74 (s, 3H), 6.88 (dd, 1H), 7.09 (d, 1H), 7.42 (dd, 1H), 7.53 (dd, 1H), 7.89 (d, 1H), 8.58 (s, 1H), 8.73 (s, 1H), 9.04 (s, 1H), 9.38 (d, 1H), 9.60 (s, 1H); Mass spectrum: 470 (MH+).

### Example 192

### 5-chloro-4-(8-fluoroimidazo[1,2-a]pyridin-3-yl)-N-[2-methoxy-5-(9-oxa-3,7-diazabicyclo[3.3.1]nonan-7-yl)phenyl]pyrimidin-2-amine

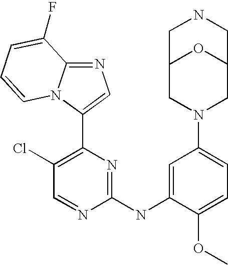

This compound was prepared following the procedure described for Example 189, using 3-(2,5-dichloropyrimidin-4-yl)-8-fluoroimidazo[1,2-a]pyridine (Method 34) instead of 3-(2,5-dichloropyrimidin-4-yl)pyrazolo[1,5-a]pyridine as a starting material; NMR spectrum: 2.35 (bs, 1H), 2.90 (d, 1H), 2.94-3.00 (m, 2H), 3.01-3.09 (m, 2H), 3.57 (d, 2H), 3.62-3.70 (m, 2H), 3.74 (s, 3H), 6.84 (dd, 1H), 6.90 (dd, 1H), 7.07 (d, 1H), 7.26 (s, 1H), 7.40 (dd, 1H), 8.57 (s, 1H), 8.70 (s, 1H), 8.98 (s, 1H), 9.36 (bs, 1H); Mass spectrum: 496 (MH+).

### Example 193

### 2-[[4-[[5-chloro-4-(8-fluoroimidazo[1,2-a]pyridin-3-yl)pyrimidin-2-yl]amino]-3-methoxy-phenyl]methyl]propane-1,3-diol

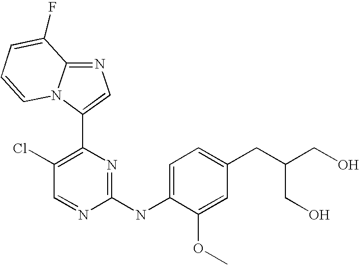

This compound was prepared following the procedure described for Example 190, using 3-(2,5-dichloropyrimidin-4-yl)-8-fluoroimidazo[1,2-a]pyridine (Method 34) instead of 3-(2,5-dichloropyrimidin-4-yl)pyrazolo[1,5-a]pyridine as a starting material; NMR spectrum: 1.76-1.88 (m, 1H), 2.60 (d, 2H), 3.36-3.46 (m, 4H), 3.77 (s, 3H), 4.43 (t, 2H), 6.80 (d, 1H), 6.88 (bs, 1H), 6.96 (s, 1H), 7.40 (dd, 1H), 7.43 (d, 1H), 8.54 (s, 1H), 8.72 (s, 1H), 8.94 (s, 1H), 9.40 (bs, 1H); Mass spectrum: 458 (MH+).

### Example 194

### 2-[4-[4-[[5-chloro-4-(8-fluoroimidazo[1,2-a]pyridin-3-yl)pyrimidin-2-yl]amino]-3-methoxy-phenyl]piperazin-1-yl]ethanol

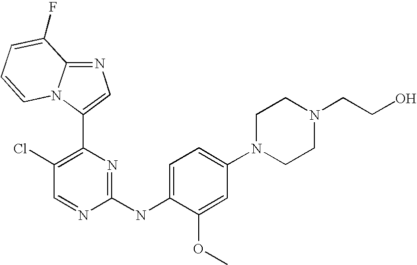

This compound was prepared following the procedure described for Example 3.45, using 3-(2,5-dichloropyrimidin-4-yl)-8-fluoroimidazo[1,2-a]pyridine (Method 34) instead of 3-(2,5-dichloropyrimidin-4-yl)imidazo[1,2-a]pyridine as a starting material; NMR spectrum: 2.46 (t partially hidden by DMSO-d6, 2H), 2.56-2.66 (m, 4H), 3.13-3.26 (m, 4H), 3.52-3.66 (m, 2H), 3.75 (s, 3H), 4.46 (t, 1H), 6.53 (d, 1H), 6.70 (s, 1H), 6.84 (bs, 1H), 7.25 (d, 1H), 7.43 (dd, 1H), 8.50 (s, 1H), 8.72 (s, 1H), 8.86 (s, 1H), 9.34 (bs, 1H); Mass spectrum: 498 (MH+).

### Example 195

### 2-[4-[4-[[5-fluoro-4-(8-fluoroimidazo[1,2-a]pyridin-3-yl)pyrimidin-2-yl]amino]-3-methoxy-phenyl]piperazin-1-yl]ethanol

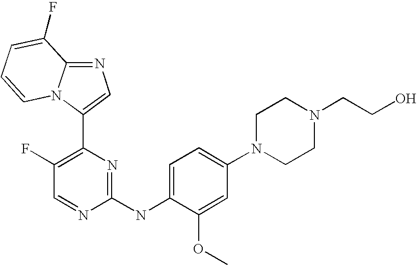

This compound was prepared following the procedure described for Example 81, using 3-(2-chloro-5-fluoropyrimidin-4-yl)-8-fluoroimidazo[1,2-a]pyridine instead of 3-(2-chloro-5-fluoropyrimidin-4-yl)imidazo[1,2-a]pyridine as a starting material; NMR spectrum: 2.47 (t, 2H), 2.57-2.63 (m, 4H), 3.17-3.23 (m, 4H), 3.54-3.60 (m, 2H), 3.76 (s, 3H), 4.46 (t, 1H), 6.55 (dd, 1H), 6.70 (d, 1H), 6.80 (bs, 1H), 7.31 (d, 1H), 7.45 (dd, 1H), 8.37 (d, 1H), 8.49 (d, 1H), 8.68 (s, 1H), 9.59 (bs, 1H); Mass spectrum: 498 (MH+).

The 3-(2-chloro-5-fluoropyrimidin-4-yl)-8-fluoroimidazo[1,2-a]pyridine used as a starting material was prepared according to Method 2, using 2-amino-3-fluoropyridine instead of 2-aminopyridine in the second step; NMR spectrum: CDCl3: 7.10 (ddd, 1H), 7.23 (d, 1H), 8.51 (d, 1H), 8.60 (d, 1H), 8.90 (d, 1H).

### Example 196

### 1-[4-[4-[[4-(8-fluoroimidazo[1,2-a]pyridin-3-yl)-5-methyl-pyrimidin-2-yl]amino]-3-methoxy-phenyl]-1-piperidyl]ethanone

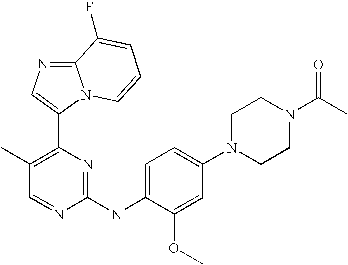

This compound was prepared following the procedure described for Example 99, using 3-(2-chloro-5-methyl-pyrimidin-4-yl)-8-fluoro-imidazo[1,2-a]pyridine instead of 3-(2-chloro-5-methylpyrimidin-4-yl)imidazo[1,2-a]pyridine; NMR spectrum: 1.45-1.66 (m, 1H), 1.60-1.71 (m, 1H), 1.76-1.87 (m, 2H), 2.05 (s, 3H), 2.40 (s, 3H), 2.55-2.63 (m, 1H), 2.72-2.82 (m, 1H), 3.09-3.19 (m, 1H), 3.83 (s, 3H), 3.91-3.99 (m, 1H), 4.53-4.60 (m, 1H), 6.82 (dd, 1H), 6.91 (ddd, 1H), 6.99 (s, 1H), 7.38 (dd, 1H), 7.69 (d, 1H), 8.30 (s, 1H), 8.35 (s, 1H), 8.40 (s, 1H), 9.43 (d, 1H); Mass spectrum: 475 (MH+).

The starting 3-(2-chloro-5-methyl-pyrimidin-4-yl)-8-fluoro-imidazo[1,2-a]pyridine was prepared according to Method 1, using 2-amino-3-fluoropyridine instead of 2-aminopyridine and (E)-4-(2-butoxyvinyl)-2-chloro-5-methylpyrimidine (see Method 44) instead of (E)-4-(2-butoxyvinyl)-2,5-dichloropyrimidine; NMR spectrum: CDCl3: 2.58 (s, 3H), 7.01 (ddd, 1H), 7.17 (dd, 1H), 8.29 (s, 1H), 8.52 (s, 1H), 9.63 (d, 1H).

### Examples 197 to 200

The following compounds were prepared according to the procedure as indicated as the “reference example” in the following table using 3-(2-chloro-5-methyl-pyrimidin-4-yl)-8-fluoro-imidazo[1,2-a]pyridine (see Example 196) instead of 3-(2,5-dichloropyrimidin-4-yl)imidazo[1,2-a]pyridine.

| | | | | Reference |
|---|---|---|---|---|
| Ex. No. | Name | MH+ | NMR spectrum | example |
| 197 | 4-(8- | 433 | 1.53 (dd, 1H), 1.58 (dd, 1H), | 4 |
| | fluoroimidazo[1,2- | | 1.68-1.76 (m, 2H), 2.39 (s, 3H), 2.54-262 (m, 3H), | |
| | a]pyridin-3-yl)-N-[2- | | 3.00-3.07 (m, 2H), 3.81 (s, 3H), | |
| | methoxy-4-(4- | | 6.80 (dd, 1H), 6.89 (ddd, 1H), 6.95 (d, 1H), | |
| | piperidyl)phenyl]-5- | | 7.37 (d, 1H), 7.65 (d, 1H), 8.29 (s, 1H), | |
| | methyl-pyrimidin-2- | | 8.34 (s, 1H), 8.39 (s, 1H), 9.42 (d, 1H) | |
| | amine | | | |
| 198 | 1-[4-[4-[[4-(8- | 491 | 1.48-1.60 (m, 1H), 1.60-1.72 (m, 1H), | 28.5 |
| | fluoroimidazo[1,2- | | 1.76-1.87 (m, 2H), 2.39 (s, 3H), | |
| | a]pyridin-3-yl)-5- | | 2.66-2.74 (m, 1H), 2.74-2.85 (m, 1H), | |
| | methyl-pyrimidin-2- | | 3.01-3.12 (m, 1H), 3.78 (bs, 1H), 3.82 (s, | |
| | yl]amino]-3-methoxy- | | 3H), 4.10 (dd, 1H), 4.16 (dd, 1H), | |
| | phenyl]-1-piperidyl]- | | 4.43-4.53 (m, 2H), 6.81 (dd, 1H), 6.91 (ddd, | |
| | 2-hydroxy-ethanone | | 1H), 6.98 (s, 1H), 7.37 (dd, 1H), 7.69 (d, | |
| | | | 1H), 8.29 (s, 1H), 8.34 (s, 1H), 8.40 (s, | |
| | | | 1H), 9.42 (d, 1H) | |
| 199 | 4-(8- | 434 | 2.39 (s, 3H), 2.74-2.81 (m, 4H), | 3.10 |
| | fluoroimidazo[1,2- | | 2.84-2.90 (m, 4H), 3.77 (s, 3H), 6.62 (dd, | |
| | a]pyridin-3-yl)-N-(2- | | 1H), 6.94 (ddd, 1H), 6.96 (d, 1H), | |
| | methoxy-5-piperazin- | | 7.37 (dd, 1H), 7.57 (d, 1H), 8.26 (s, 1H), | |
| | 1-yl-phenyl)-5- | | 8.28 (s, 1H), 8.45 (s, 1H), 8.33 (s, 0.5H), | |
| | methyl-pyrimidin-2- | | 8.35 (s, 0.5H) | |
| | amine | | | |
| 200 | (2R)-1-[4-[4-[[4-(8- | 505 | 1.20 (d, 1.5H), 1.23 (d, 1.5H), | 28.2 |
| | fluoroimidazo[1,2- | | 1.44-1.72 (m, 2H), 1.79-1.90 (m, 2H), 2.39 (s, | |
| | a]pyridin-3-yl)-5- | | 3H), 2.63-2.72 (m, 1H), 2.76-2.85 (m, | |
| | methyl-pyrimidin-2- | | 1H), 3.05-3.16 (m, 1H), 3.82 (s, 3H), | |
| | yl]amino]-3-methoxy- | | 4.06-4.17 (m, 1H), 4.42-4.60 (m, 2H), | |
| | phenyl]-1-piperidyl]- | | 4.79 (d, 0.5H), 4.86 (d, 0.5H), 6.82 (d, | |
| | 2-hydroxy-propan-1- | | 1H), 6.90 (ddd, 1H), 6.98 (s, 1H), | |
| | one | | 7.37 (dd, 1H), 7.69 (d, 1H), 8.29 (s, 1H), | |
| | | | 8.34 (s, 1H), 8.40 (s, 1H), 9.42 (d, 1H) | |

### Example 201

### 1-[4-[4-[[5-cyclopropyl-4-(8-fluoroimidazo[1,2-a]pyridin-3-yl)pyrimidin-2-yl]amino]-3-methoxy-phenyl]piperazin-1-yl]ethanone

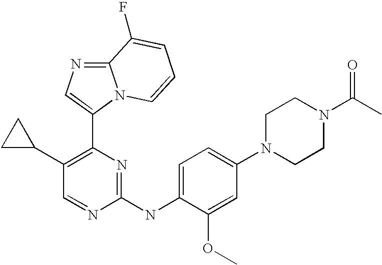

This compound was prepared following the procedure described for Example 126.14, using 3-(2-chloro-5-cyclopropyl-pyrimidin-4-yl)-8-fluoro-imidazo[1,2-a]pyridine instead of 3-(2,5-dichloro pyrimidin-4-yl)-8-fluoroimidazo[1,2-a]pyridine; NMR spectrum: 0.61-0.71 (m, 2H), 0.97-1.06 (m, 2H), 1.97-2.05 (m, 1H), 2.06 (s, 3H), 3.09-3.16 (m, 2H), 3.16-3.22 (m, 2H), 3.54-3.67 (m, 4H), 3.77 (s, 3H), 6.54 (dd, 1H), 6.73 (d, 1H), 6.84 (dd, 1H), 7.36 (dd, 1H) 7.41 (d, 1H), 8.29 (s, 1H), 8.38 (s, 1H), 8.59 (s, 1H), 9.49 (bs, 1H); Mass spectrum: 502 (MH+).

The starting 3-(2-chloro-5-cyclopropyl-pyrimidin-4-yl)-8-fluoro-imidazo[1,2-a]pyridine was prepared according to Method 1, using 2-amino-3-fluoropyridine instead of 2-aminopyridine and (E)-4-(2-butoxyvinyl)-2-chloro-5-cyclopropylpyrimidine instead of (E)-4-(2-butoxyvinyl)-2,5-dichloropyrimidine; NMR spectrum: CDCl3: 0.81-0.90 (m, 2H), 1.21-1.32 (m, 2H), 2.01-2.11 (m, 1H), 7.02 (ddd, 1H), 7.18 (dd, 1H), 8.46 (s, 1H), 8.70 (s, 1H), 9.67 (d, 1H).

### Preparation of (E)-4-(2-butoxyvinyl)-2-chloro-5-cyclopropylpyrimidine

Triethylamine (2.28 ml, 16.4 mmol), 1-(vinyloxy)butane (19.3 ml, 149 mmol) and palladium(II) acetate (0.235 g, 1.05 mmol) were added to a stirred solution of 2,4-dichloro-5-cyclopropylpyrimidine (2.83 g, 14.9 mmol) in polyethylene glycol 400 (25 ml, 14.9 mmol). This mixture is heated at 80° C. for 6 hours then cooled and diethylether is added under stirring. The organic layer is separated by decantation and this operation is repeated twice. The organic extracts were washed with brine, dried over MgSO₄ and evaporated to afford an orange oil. This crude product was purified by flash chromatography on silica gel eluting with 0 to 10% ethyl acetate in petroleum ether to afford (E)-4-(2-butoxyvinyl)-2-chloro-5-cyclopropylpyrimidine (1.78 g, 47%) as a pale yellow liquid; NMR spectrum: CDCl3: 0.59-3.68 (m, 2H), 0.97 (s, 3H), 0.97-1.04 (m, 2H), 1.40-1.51 (m, 2H), 1.66-1.78 (m, 3H), 4.02 (t, 2H), 6.21 (d, 1H), 7.98 (d, 1H), 8.11 (s, 1H).

### Example 202

### 1-[4-[4-[[5-cyclopropyl-4-(8-fluoroimidazo[1,2-a]pyridin-3-yl)pyrimidin-2-yl]amino]-3-methoxy-phenyl]-1-piperidyl]ethanone

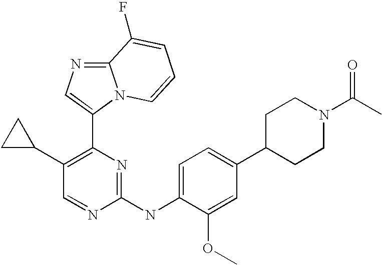

This compound was prepared following the procedure described for Example 201, using 1-[4-(4-amino-3-methoxy-phenyl)-1-piperidyl]ethanone instead of 1-[4-(4-amino-3-methoxy-phenyl)piperazin-1-yl]ethanone; NMR spectrum: 0.64-0.72 (m, 2H), 0.99-1.05 (m, 2H), 21.46-1.56 (m, 1H), 1.60-1.71 (m, 1H), 1.76-1.87 (m, 2H), 2.00-2.07 (m, 1H), 2.04 (s, 3H), 2.55-2.63 (m, 1H), 2.73-2.82 (m, 1H), 3.09-3.17 (m, 1H), 3.81 (s, 3H), 3.91-3.98 (m, 1H), 4.53-4.60 (m, 1H), 6.82 (dd, 1H), 6.89 (ddd, 1H), 6.99 (d, 1H), 7.37 (dd, 1H), 7.64 (d, 1H), 8.33 (s, 1H), 8.43 (s, 1H), 8.59 (s, 1H), 9.52 (d, 1H); Mass spectrum: 501 (MH+).

### Example 203

### 5-chloro-4-(8-fluoroimidazo[1,2-a]pyridin-3-yl)-N-[2-methoxy-4-(7-methyl-9-oxa-3,7-diazabicyclo[3.3.1]nonan-3-yl)phenyl]pyrimidin-2-amine

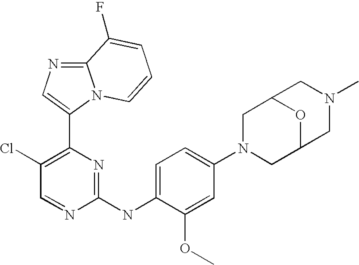

Sodium triacetoxyhydroborate (0.128 g, 0.60 mmol) was added portionwise at 25° C. to a mixture of formaldehyde (0.045 ml, 0.60 mmol) and N-(4-(9-oxa-3,7-diazabicyclo[3.3.1]nonan-3-yl)-2-methoxyphenyl)-5-chloro-4-(8-fluoroimidazo[1,2-a]pyridin-3-yl)pyrimidin-2-amine (Example 183, 0.2 g, 0.40 mmol) in methanol (3.5 ml) and methylene chloride (3.5 ml). The reaction mixture was stirred for 2 hours then a solution of ammonia in methanol (7N, 5 ml) was added and the solvents were removed under vacuum. The crude product was purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford a pale yellow foam which was triturated with diethylether to give 5-chloro-4-(8-fluoroimidazo[1,2-a]pyridin-3-yl)-N-(2-methoxy-4-(7-methyl-9-oxa-3,7-diazabicyclo[3.3.1]nonan-3-yl)phenyl)pyrimidin-2-amine (0.100 g, 48%) as a pale yellow solid; NMR spectrum: 2.11 (s, 3H), 2.26-2.39 (m, 2H), 3.83-3.94 (m, 2H), 3.0463.14 (m, 2H), 3.64-3.72 (m, 2H), 3.75 (s, 3H), 4.07 (s, 2H), 6.39 (d, 1H), 6.53 (s, 1H), 6.82 (bs, 1H), 7.21 (d, 1H), 7.39 (dd, 1H), 8.47 (s, 1H), 8.73 (s, 1H), 8.83 (s, 1H), 9.27 (bs, 1H); Mass spectrum: 510 (MH+).

### Example 204

### 3-[7-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-3-yl]propan-1-ol

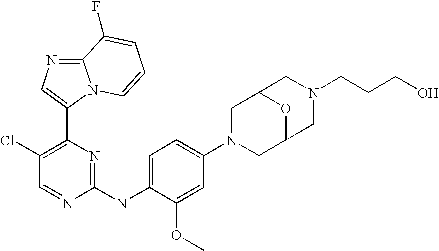

A mixture of N-(4-(9-oxa-3,7-diazabicyclo[3.3.1]nonan-3-yl)-2-methoxyphenyl)-5-chloro-4-(8-fluoroimidazo[1,2-a]pyridin-3-yl)pyrimidin-2-amine (Example 183, 100 mg, 0.20 mmol), 3-bromopropan-1-ol (21 μL, 0.22 mmol) and potassium carbonate (56 mg, 0.4 mmol) in DMF (1 mL) was stirred at room temperature for 4 hours. The reaction mixture was filtered and purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford 3-(7-(4-(5-chloro-4-(8-fluoroimidazo[1,2-a]pyridin-3-yl)pyrimidin-2-ylamino)-3-methoxyphenyl)-9-oxa-3,7-diazabicyclo[3.3.1]nonan-3-yl)propan-1-ol (44 mg, 79%) as a foam; NMR spectrum: 1.53-1.63 (m, 2H), 2.31 (t, 2H), 2.34 (dd, 2H), 3.04 (d, 2H), 3.08 (dd, 2H), 3.39-3.47 (m, 2H), 3.66 (d, 2H), 3.74 (s, 3H), 4.07 (bs, 2H), 4.66 (bs, 1H), 6.41 (dd, 1H), 6.54 (d, 1H), 6.85 (bs, 1H), 7.20 (d, 1H), 7.40 (dd, 1H), 8.48 (s, 1H), 8.74 (s, 1H), 8.83 (s, 1H), 9.32 (bs, 1H); Mass spectrum: 554 (MH+).

### Example 205

### 2-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-N,N-dimethyl-acetamide

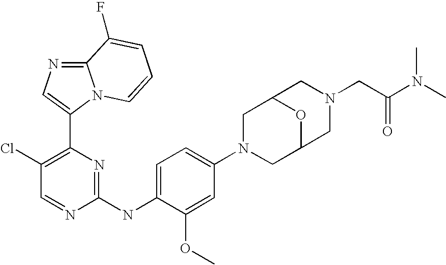

A mixture of N-(4-(9-oxa-3,7-diazabicyclo[3.3.1]nonan-3-yl)-2-methoxyphenyl)-5-chloro-4-(8-fluoroimidazo[1,2-a]pyridin-3-yl)pyrimidin-2-amine (Example 183, 120 mg, 0.24 mmol), 2-chloro-N,N-dimethylacetamide (0.035 mL, 0.34 mmol) and potassium carbonate (33 mg, 0.24 mmol) in DMF (2 mL) was stirred at 25° C. for 16 hours. The reaction mixture was purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford 2-(7-(4-(5-chloro-4-(8-fluoroimidazo[1,2-a]pyridin-3-yl)pyrimidin-2-ylamino)-3-methoxyphenyl)-9-oxa-3,7-diazabicyclo[3.3.1]nonan-3-yl)-N,N-dimethylacetamide (72 mg, 51%) as a yellow solid foam; NMR spectrum: 2.49 (bs partially hidden by H2O, 2H), 2.71 (s, 3H), 2.82 (s, 3H), 2.86-2.93 (m, 2H), 3.02 (s, 2H), 3.08-3.17 (m, 2H), 3.36-3.72 (m, 2H), 3.76 (s, 3H), 4.03-4.10 (m, 2H), 6.41 (d, 1H), 6.55 (s, 1H), 6.81 (bs, 1H), 7.22 (d, 1H), 7.42 (dd, 1H), 8.49 (s, 1H), 8.73 (s, 1H), 8.83 (s, 1H), 9.34 (bs, 1H); Mass spectrum: 581 (MH+).

### Example 206

### 3-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-N-methyl-propanamide

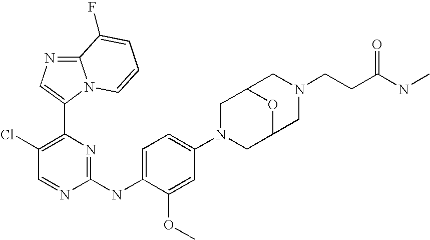

A mixture of N-(4-(9-oxa-3,7-diazabicyclo[3.3.1]nonan-3-yl)-2-methoxyphenyl)-5-chloro-4-(8-fluoroimidazo[1,2-a]pyridin-3-yl)pyrimidin-2-amine (Example 183, 100 mg, 0.20 mmol), 3-bromopropanoic acid (40 mg, 0.26 mmol) and N-ethyl-N-isopropylpropan-2-amine (0.17 mL, 1.0 mmol) in DMF (1 mL) was stirred at room temperature overnight. Methanamine hydrochloride (14 mg, 0.20 mmol) and 2-(3H-[1,2,3]triazolo[4,5-b]pyridin-3-yl)-1,1,3,3-tetramethylisouronium hexafluorophosphate(V) (77 mg, 0.20 mmol) were added and reaction mixture was stirred at room temperature for 3 hours. The reaction mixture was purified by preparative HPLC using a Waters X-Bridge reverse-phase column (5 microns silica, 30 mm diameter, 150 mm length) and decreasingly polar mixtures of water (containing 1% acetic acid) and acetonitrile as eluent. The collected fractions were combined, neutralised with NaOH 2N and extracted with methylene chloride. The organic extracts were dried over MgSO4 and evaporated to dryness. The product was further purified by flash chromatography on silica gel eluting with 0 to 4% methanolic ammonia (7 N) in dichloromethane to afford 3-(7-(4-(5-chloro-4-(8-fluoroimidazo[1,2-a]pyridin-3-yl)pyrimidin-2-ylamino)-3-methoxyphenyl)-9-oxa-3,7-diazabicyclo[3.3.1]nonan-3-yl)-N-methylpropanamide (33.0 mg, 56%); NMR spectrum: 2.03 (d, 3H), 2.17-2.24 (m, 2H), 2.37-2.43 (m, 4H), 3.02-3.111 (m, 4H), 3.76 (d, 2H), 3.77 (s, 3H), 4.03-4.14 (m, 2H), 6.48 (dd, 1H), 6.61 (d, 1H), 6.85 (bs, 1H), 7.28 (d, 1H), 7.40 (dd, 1H), 8.30 (dd, 1H), 8.48 (s, 1H), 8.71 (s, 1H), 8.85 (s, 1H), 9.38 (bs, 1H); Mass spectrum: 581 (MH+).

### Example 207

### 5-chloro-4-(8-fluoroimidazo[1,2-a]pyridin-3-yl)-N-[2-methoxy-4-[3-(2-methylsulfonylethyl)-9-oxa-3,7-diazabicyclo[3.3.1]nonan-7-yl]phenyl]pyrimidin-2-amine

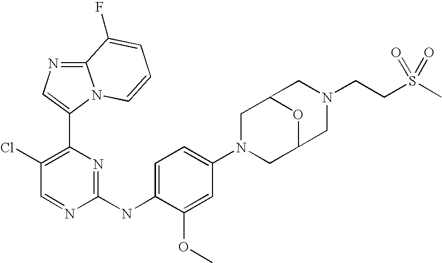

A mixture of N-(4-(9-oxa-3,7-diazabicyclo[3.3.1]nonan-3-yl)-2-methoxyphenyl)-5-chloro-4-(8-fluoroimidazo[1,2-a]pyridin-3-yl)pyrimidin-2-amine (Example 183, 120 mg, 0.24 mmol), ammonium cerium IV nitrate (13.2 mg, 0.02 mmol) and methylsulfonylethene (51 mg, 0.48 mmol) in water (1 mL) and THF (1 mL) was stirred at 25° C. overnight. The reaction mixture was extracted with methylene chloride (2×10 ml) and the combined organic extracts were dried over magnesium sulfate and concentrated. The crude product was purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford 5-chloro-4-(8-fluoroimidazo[1,2-a]pyridin-3-yl)-N-(2-methoxy-4-(7-(2-(methylsulfonyl)ethyl)-9-oxa-3,7-diazabicyclo[3.3.1]nonan-3-yl)phenyl)pyrimidin-2-amine (75 mg, 51%) as a beige solid; NMR spectrum: 2.48-2.58 (m, 2H), 2.72-2.78 (m, 2H), 2.97 (s, 3H), 3.17-3.28 (m, 4H), 3.30-3.38 (m, 2H), 3.79-3.88 (m, 2H), 3.91 (s, 3H), 4.22-4.30 (m, 2H), 6.52 (d, 1H), 6.56 (s, 1H), 6.99 (bs, 1H), 7.39 (d, 1H), 7.54 (dd, 1H), 8.64 (s, 1H), 8.87 (s, 1H), 8.97 (s, 1H), 9.52 (bs, 1H); Mass spectrum: 602 (MH+).

### Example 208

### 1-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-2-(methylamino)ethanone

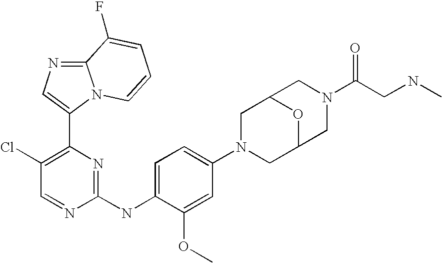

This compound was prepared following the procedure described for Example 140, using N-(4-(9-oxa-3,7-diazabicyclo[3.3.1]nonan-3-yl)-2-methoxyphenyl)-5-chloro-4-(8-fluoroimidazo[1,2-a]pyridin-3-yl)pyrimidin-2-amine (Example 183) instead of 5-chloro-4-(imidazo[1,2-a]pyridin-3-yl)-N-(2-methoxy-4-(piperidin-4-yl)phenyl)pyrimidin-2-amine; NMR spectrum: 1.62 (bs, 1H), 2.07 (s, 3H), 2.98-3.07 (m, 2H), 3.10 (dd, 1H), 3.19-3.28 (m, 2H), 3.54 (dd, 1H), 3.64 (d, 1H), 3.74 (s, 3H), 3.76 (d, 1H), 3.90-4.10 (m, 4H), 4.48 (d, 1H), 6.45 (dd, 1H), 6.60 (d, 1H), 6.80 (bs, 1H), 7.22 (d, 1H), 7.43 (dd, 1H), 8.48 (s, 1H), 8.73 (s, 1H), 8.85 (s, 1H), 9.32 (bs, 1H); Mass spectrum: 567 (MH+).

### Example 209

### 2-amino-1-[7-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-3-yl]ethanone

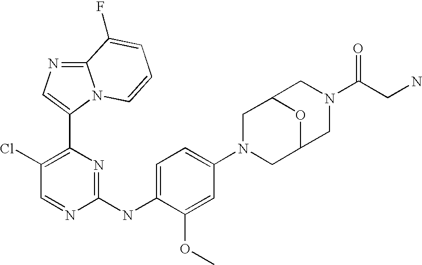

This compound was prepared following the procedure described for Example 208, using 2-(tert-butoxycarbonylamino)acetic acid instead of 2-(tert-butoxycarbonyl(methyl)amino)acetic acid; NMR spectrum: 1.61 (bs, 2H), 2.99-3.07 (m, 2H), 3.11 (dd, 1H), 3.28 (s, 2H), 3.53 (dd, 1H), 3.62 (d, 1H), 3.74 (s, 3H), 3.93 (dd, 2H), 4.01 (dd, 1H), 4.07 (dd, 1H), 4.47 (d, 1H), 6.46 (dd, 1H), 6.80 (d, 1H), 6.85 (bs, 1H), 7.21 (d, 1H), 7.43 (dd, 1H), 8.49 (s, 1H), 8.73 (s, 1H), 8.86 (s, 1H), 9.31 (bs, 1H); Mass spectrum: 553 (MH+).

### Example 210

### (2S)-2-amino-1-[7-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-3-yl]-3-hydroxy-propan-1-one

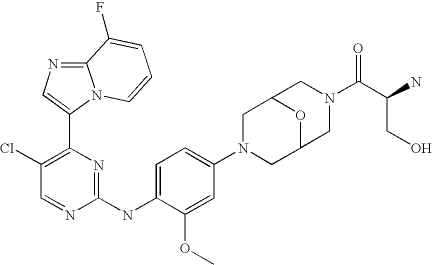

This compound was prepared following the procedure described for Example 208, using (S)-2-(tert-butoxycarbonylamino)-3-hydroxypropanoic acid instead of 2-(tert-butoxycarbonyl(methyl)amino)acetic acid; NMR spectrum: (CDCl₃) 1.72 (bs, 3H), 3.17-3.37 (m, 3.5H), 3.38-3.44 (m, 0.5H), 3.44-3.50 (m, 0.5H), 3.50-3.55 (m, 0.5H), 3.56-3.65 (m, 1.5H), 3.65-3.70 (m, 1H), 3.71-3.81 (m, 1.5H), 3.88 (s, 1.5H), 3.89 (s, 1.5H), 4.02-4.14 (m, 2.5H), 4.17-4.22 (m, 0.5H), 4.69-4.79 (m, 1H), 6.41 (dd, 1H), 6.47 (dd, 1H), 6.83 (dd, 1H), 7.10 (dd, 1H), 7.34 (s, 0.5H), 7.39 (s, 0.5H), 7.96 (d, 0.5H), 8.00 (d, 0.5H), 8.44 (s, 1H), 8.74 (s, 0.5H), 8.75 (s, 0.5H), 9.39 (s, 0.5H), 9.40 (s, 0.5H); Mass spectrum: 583 (MH+).

### Example 211

### 1-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-2-(dimethylamino)ethanone

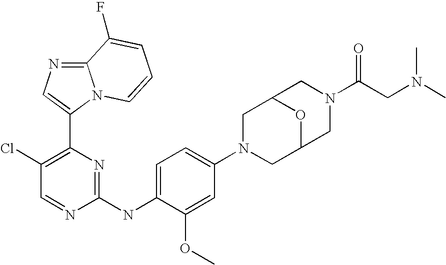

2-(3H-[1,2,3]triazolo[4,5-b]pyridin-3-yl)-1,1,3,3-tetramethylisouronium hexafluorophosphate(V) (98 mg, 0.26 mmol) was added to a stirred solution of N-(4-(9-oxa-3,7-diazabicyclo[3.3.1]nonan-3-yl)-2-methoxyphenyl)-5-chloro-4-(8-fluoroimidazo[1,2-a]pyridin-3-yl)pyrimidin-2-amine (Example 183, 120 mg, 0.20 mmol), 2-(dimethylamino)acetic acid (20.4 mg, 0.20 mmol) and N-ethyl-N-isopropylpropan-2-amine (0.052 mL, 0.30 mmol) in DMF (2 mL) at 25° C. The resulting solution was stirred at 25° C. for 5 minutes then purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford 1-(7-(4-(5-chloro-4-(8-fluoroimidazo[1,2-a]pyridin-3-yl)pyrimidin-2-ylamino)-3-methoxyphenyl)-9-oxa-3,7-diazabicyclo[3.3.1]nonan-3-yl)-2-(dimethylamino)ethanone (44 mg, 38%) as a pale yellow foam; NMR spectrum: 2.04 (s, 6H), 2.89-2.95 (m, 1H), 3.01-3.14 (m, 4H), 3.52-3.59 (m, 1H), 3.67-3.73 (m, 1H), 3.75 (s, 3H), 3.87-3.95 (m, 1H), 4.07-4.13 (m, 2H), 4.25-4.33 (m, 1H), 4.43-4.52 (m, 1H), 6.48 (d, 1H), 6.82 (bs, 1H), 7.23 (d, 1H), 7.43 (dd, 1H), 8.50 (s, 1H), 8.74 (s, 1H), 8.86 (s, 1H), 9.33 (bs, 1H); Mass spectrum: 581 (MH+).

### Example 212

### 1-[7-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-3-yl]-2-hydroxy-ethanone

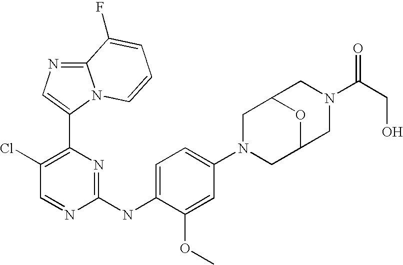

This compound was prepared following the procedure described for Example 211, using hydroxyacetic acid instead of 2-(dimethylamino)acetic acid; NMR spectrum: 2.99-3.08 (m, 2H), 3.12-3.20 (m, 1H), 3.53-3.59 (m, 1H), 3.59-3.65 (m, 1H), 3.74 (s, 3H), 3.82-3.88 (m, 1H), 3.92-4.00 (m, 1H), 4.00-4.06 (m, 2H), 4.06-4.10 (m, 2H), 4.39 (t, 1H), 4.41-4.47 (m, 1H), 6.47 (dd, 1H), 6.62 (d, 1H), 6.78 (bs, 1H), 7.22 (d, 1H), 7.43 (dd, 1H), 8.49 (s, 1H), 8.73 (s, 1H), 8.87 (s, 1H), 9.31 (bs, 1H); Mass spectrum: 554 (MH+).

### Example 213

### (2S)-1-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-2-hydroxy-propan-1-one

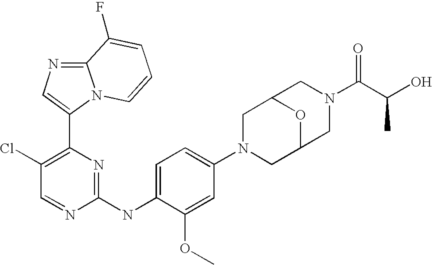

This compound was prepared following the procedure described for Example 211, using (S)-2-hydroxypropanoic acid instead of 2-(dimethylamino)acetic acid; NMR spectrum: 0.95 (d, 1.8H), 1.18 (d, 1.2H), 2.99-3.10 (m, 2H), 3.10-3.18 (m, 1H), 3.55-3.63 (1.2H), 3.64-3.69 (m, 0.8H), 3.73 (s, 1.8H), 3.74 (s, 1.2H), 3.86-3.95 (m, 1H), 4.01-4.06 (m, 1H), 4.07-4.13 (m, 1.6H), 4.14-4.20 (m, 0.4H), 4.35-4.42 (m, 0.6H), 4.43-4.50 (m, 1H), 4.50-4.55 (m, 0.4H), 4.57-4.62 (m, 0.4H), 4.71-4.77 (m, 0.6H), 6.44 (ddd, 1H), 6.60 (s, 1H), 6.78 (bs, 1H), 7.22 (d, 1H), 7.42 (ddd, 1H), 8.49 (s, 1H), 8.73 (s, 1H), 8.85 (s, 0.6H), 8.86 (s, 0.4H), 9.30 (bs, 1H); Mass spectrum: 568 (MH+).

### Example 214

### (2R)-1-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-2-hydroxy-propan-1-one

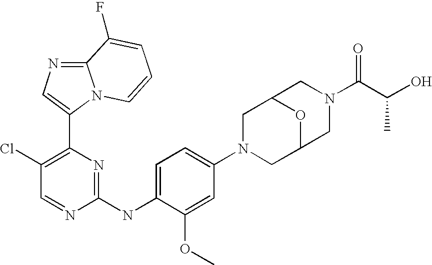

This compound was prepared following the procedure described for Example 211, using (R)-2-hydroxypropanoic acid instead of 2-(dimethylamino)acetic acid; NMR spectrum: 0.96 (d, 1.8H), 1.16 (d, 1.2H), 2.99-3.10 (m, 2H), 3.10-3.20 (m, 1H), 3.55-3.63 (m, 1.4H), 3.67 (d, 0.6H), 3.73 (s, 1.8H), 3.74 (s, 1.2H), 3.86-3.95 (m, 1H), 4.01-4.07 (m, 1H), 4.06-4.14 (m, 1.6H), 4.17 (d, 0.4H), 4.35-4.43 (m, 0.6H), 4.43-4.50 (m, 1H), 4.50-4.55 (m, 0.4H), 4.58 (d, 0.4H), 4.76 (d, 0.6H), 6.44 (ddd, 1H), 6.60 (dd, 1H), 6.78 (bs, 1H), 7.22 (d, 1H), 7.42 (dd, 1H), 8.49 (s, 1H), 8.74 (s, 1H), 8.86 (d, 1H), 9.30 (bs, 1H); Mass spectrum: 568 (MH+).

### Example 215

### 1-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]ethanone

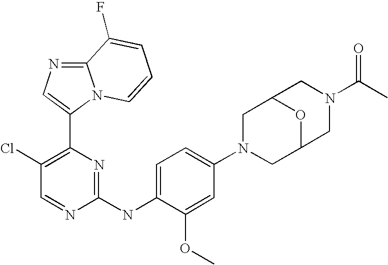

A mixture of 3-(2,5-dichloropyrimidin-4-yl)-8-fluoroimidazo[1,2-a]pyridine (Method 34, 0.15 g, 0.53 mmol), 1-(7-(4-amino-3-methoxyphenyl)-9-oxa-3,7-diazabicyclo[3.3.1]nonan-3-yl)ethanone (0.154 g, 0.53 mmol) and 4-methylbenzenesulfonic acid hydrate (0.101 g, 0.53 mmol) in 2-pentanol (1.5 ml) was heated to 140° C. for two hours in a sealed tube in a microwave reactor. After dilution with diethylether, the precipitated yellow solid was filtered then stirred in acetic anhydride at 60° C. After one hour, Ac2O was evaporated and the residue dried under vacuum. This crude product was dissolved in MeOH (5 ml) and THF (5 ml). Four drops of a 10N aqueous solution of NaOH were added. After one hour, a saturated aqueous solution of sodium bicarbonate was added and the mixture was extracted with methylene chloride. The organic extracts were dried over magnesium sulfate, filtered and evaporated to dryness to afford the crude product which was triturated in methylene chloride to provide the desired compound (0.100 g, 35%) as a pale yellow solid; NMR spectrum: (323° K) 1.92 (s, 3H), 3.01-3.14 (m, 3H), 3.63 (d, 2H), 3.75 (s, 3H), 3.92 (d, 1H), 3.96-4.09 (m, 3H), 4.51 (d, 1H), 6.49 (d, 1H), 6.62 (s, 1H), 6.81 (bs, 1H), 7.25 (d, 1H), 7.38 (dd, 1H), 8.49 (s, 1H), 8.71 (s, 1H), 8.73 (s, 1H), 9.32 (bs, 1H); Mass spectrum: 538 (MH+).

The 1-(7-(4-amino-3-methoxyphenyl)-9-oxa-3,7-diazabicyclo[3.3.1]nonan-3-yl)ethanone used as a starting material was prepared as follows:

2,3,4,6,7,8,9,10-octahydropyrimido[1,2-a]azepine (DBU) (0.75 ml, 5.0 mmol) was added to a stirred suspension of 4-(9-oxa-3,7-diazabicyclo[3.3.1]nonan-3-yl)-2-methoxyaniline (Method 42, 0.5 g, 2.01 mmol) in ethyl acetate (5 ml) and dichloromethane (5 ml) at room temperature. The resulting solution was stirred at room temperature for 10 minutes. 1,1′-(2-oxo-1H-benzo[d]imidazole-1,3(2H)-diyl)diethanone (0.438 g, 2.0 mmol) was then added and reaction stirred at room temperature for 1 hour. Ethyl acetate (30 ml) was added to the reaction mixture, the mixture was filtered and the filtrate was concentrated. The crude product was purified by flash chromatography on silica gel eluting with 0 to 5% methanol in ethyl acetate/dichloromethane 50/50 to afford 1-(7-(4-amino-3-methoxyphenyl)-9-oxa-3,7-diazabicyclo[3.3.1]nonan-3-yl)ethanone (0.295 g, 50%) as a white solid; NMR spectrum: 1.90 (s, 3H), 2.84-2.94 (m, 2H), 2.95-3.03 (m, 1H), 3.28-3.33 (m partially hidden by H2O, 1H), 3.52-3.59 (m, 1H), 3.59-3.64 (m, 1H), 3.73 (s, 3H), 3.88-3.97 (m, 3H), 4.18 (bs, 2H), 4.40-4.46 (m, 1H), 6.21 (dd, 1H), 6.42 (d, 1H), 6.51 (d, 1H).

### Example 216

### 2-[4-[4-[[5-chloro-4-(8-fluoroimidazo[1,2-a]pyridin-3-yl)pyrimidin-2-yl]amino]-3-methoxy-phenyl]-1-piperidyl]-N,N-dimethyl-acetamide

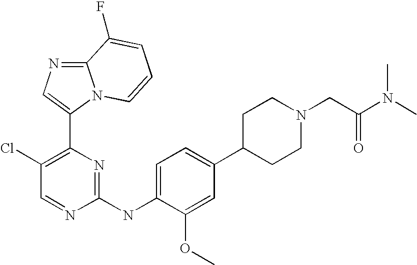

A suspension of 5-chloro-4-(8-fluoroimidazo[1,2-a]pyridin-3-yl)-N-(2-methoxy-4-(piperidin-4-yl)phenyl)pyrimidin-2-amine (prepared following the same procedure as for 5-chloro-4-(imidazo[1,2-a]pyridin-3-yl)-N-(2-methoxy-4-(piperidin-4-yl)phenyl)pyrimidin-2-amine, Example 4, using 3-(2,5-dichloropyrimidin-4-yl)-8-fluoroimidazo[1,2-a]pyridine as a starting material) (150 mg, 0.30 mmol), cesium carbonate (107 mg, 0.33 mmol) and 2-chloro-N,N-dimethylacetamide (0.031 ml, 0.30 mmol) in DMF (1.5 ml) was stirred at 50° C. for 3 hours. The reaction mixture was purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent to afford 2-(4-(4-(5-chloro-4-(8-fluoroimidazo[1,2-a]pyridin-3-yl)pyrimidin-2-ylamino)-3-methoxyphenyl)piperidin-1-yl)-N,N-dimethylacetamide (86 mg, 53%); NMR spectrum: 1.66-1.82 (m, 4H), 2.11-2.20 (m, 2H), 2.51-2.57 (m partially hidden by DMSOd5, 1H), 2.82 (s, 3H), 2.91-3.01 (m, 2H), 3.06 (s, 3H), 3.15 (s, 2H), 3.79 (s, 3H), 6.85 (dd, 1H), 6.87 (dd, 1H), 7.43 (dd, 1H), 7.46 (d, 1H), 8.54 (s, 1H), 8.72 (s, 1H), 8.94 (s, 1H), 9.41 (bs, 1H); Mass spectrum: 538 (MH+).

### Example 217

### 2-[4-[[5-chloro-4-(8-fluoroimidazo[1,2-a]pyridin-3-yl)pyrimidin-2-yl]amino]-3-methoxy-phenoxy]-N,N-dimethyl-acetamide

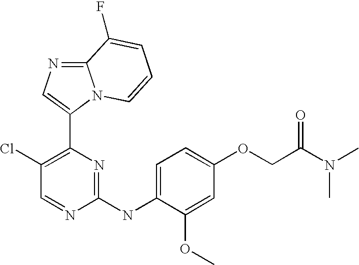

3-(2,5-dichloropyrimidin-4-yl)-8-fluoroimidazo[1,2-a]pyridine (Method 34, 116 mg, 0.39 mmol), 2-(4-amino-3-methoxyphenoxy)-N,N-dimethylacetamide (88 mg, 0.39 mmol), diacetoxypalladium (3.5 mg, 0.02 mmol), (9,9-dimethyl-9H-xanthene-4,5-diyl)bis(diphenylphosphine) (18.1 mg, 0.03 mmol) and cesium carbonate (192 mg, 0.59 mmol) were suspended in 1,4-dioxane (1 mL) and sealed into a microwave tube. The reaction was degassed, purged with nitrogen and heated to 90° C. over a period of 30 minutes in the microwave reactor. The reaction mixture was filtered and washed with CH2Cl2/MeOH 9/1. the filtrate was concentrated to dryness and the crude product was purified by flash chromatography on silica gel eluting with 0 to 4% methanol in methylene chloride to afford 2-(4-(5-chloro-4-(8-fluoroimidazo[1,2-a]pyridin-3-yl)pyrimidin-2-ylamino)-3-methoxyphenoxy)-N,N-dimethylacetamide (7 mg, 4%) as a yellow solid; NMR spectrum: 2.87 (s, 3H), 3.03 (s, 3H), 3.74 (s, 3H), 4.84 (s, 2H), 6.53 (dd, 1H), 6.74 (d, 1H), 6.94 (bs, 1H), 7.30 (d, 1H), 7.40 (dd, 1H), 8.50 (s, 1H), 8.73 (s, 1H), 8.94 (s, 1H), 9.33 (bs, 1H); Mass spectrum: 568 (MH+).

The 2-(4-amino-3-methoxyphenoxy)-N,N-dimethylacetamide used as a starting material was prepared as follows:

A solution of (E)-di-tert-butyl diazene-1,2-dicarboxylate (1.02 g, 4.43 mmol) in methylene chloride (4 mL) was added to a stirred suspension of 3-methoxy-4-nitrophenol (500 mg, 2.96 mmol), methyl 2-hydroxyacetate (0.228 mL, 2.96 mmol) and triphenylphosphine (1.16 g, 4.43 mmol) in methylene chloride (6 mL) at room temperature. The resulting solution was stirred for 2 hours and the reaction mixture were concentrated to dryness. The crude product was purified by flash chromatography on silica gel eluting with 50 to 90% methylene chloride in petroleum ether to afford methyl 2-(3-methoxy-4-nitrophenoxy)acetate (530 mg, 74%) as a pale yellow solid.

A mixture of methyl 2-(3-methoxy-4-nitrophenoxy)acetate (568 mg, 2.35 mmol) and sodium hydroxide 6N (1.57 mL, 9.42 mmol) in MeOH (7 mL) was stirred at room temperature for 2 hours. The white precipitate was filtered off, washed with ether and dried under high vacuum over P2O5 at room temperature to afford sodium 2-(3-methoxy-4-nitrophenoxy)acetate (515 mg, 88%) as a pale yellow solid.

2-(3H-[1,2,3]triazolo[4,5-b]pyridin-3-yl)-1,1,3,3-tetramethylisouronium hexafluorophosphate(V) (862 mg, 2.27 mmol) was added to a stirred suspension of 2-(3-methoxy-4-nitrophenoxy)acetic acid, sodium salt (436 mg, 1.74 mmol), dimethylamine hydrochloride (156 mg, 1.92 mmol) and N-ethyl-N-isopropylpropan-2-amine (0.395 mL, 2.27 mmol) in methylene chloride (5 mL) at room temperature. The resulting suspension was stirred for 1.5 hours. The precipitate was filtered and the filtrate was diluted with methylene chloride (5 mL) and washed with water. The aqueous layer was extracted with methylene chloride. The combined organic extracts were dried over MgSO4 and concentrated to dryness. The crude product was purified by flash chromatography on silica gel eluting with 0 to 30% ethyl acetate in methylene chloride to afford 2-(3-methoxy-4-nitrophenoxy)-N,N-dimethylacetamide (185 mg, 42%) as pale yellow oil which crystrallised on standing.

A suspension of 2-(3-methoxy-4-nitrophenoxy)-N,N-dimethylacetamide (200 mg, 0.79 mmol) and platinum(IV) oxide (20 mg, 0.09 mmol) in ethyl acetate (3 mL) and ethanol (6 mL) at room temperature, was hydrogenated under 1700 mbar at room temperature for 1 hour. The resulting suspension was filtered, washed with ethyl acetate and the filtrate was concentrated to dryness to afford crude 2-(4-amino-3-methoxyphenoxy)-N,N-dimethylacetamide. This crude product was purified by flash chromatography on silica gel eluting with 10 to 70% ethyl acetate in methylene chloride to afford 2-(4-amino-3-methoxyphenoxy)-N,N-dimethylacetamide (128 mg, 72%) as a pale purple solid; NMR spectrum: CDCl₃: 2.64 (bs, 2H), 2.98 (s, 3H), 3.08 (s, 3H), 3.83 (s, 3H), 4.61 (s, 2H), 6.36 (dd, 1H), 6.56 (d, 1H), 6.63 (d, 1H).

### Example 218

### (2S)-2-[4-[[5-chloro-4-(8-fluoroimidazo[1,2-a]pyridin-3-yl)pyrimidin-2-yl]amino]-3-methoxy-phenoxy]-N-methyl-propanamide

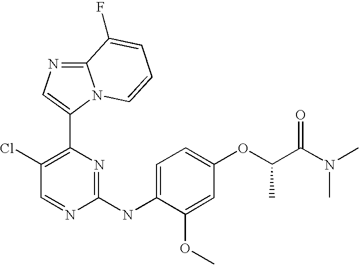

A mixture of 3-(2,5-dichloropyrimidin-4-yl)-8-fluoroimidazo[1,2-a]pyridine (Method 34, 36 mg, 0.12 mmol), (S)-2-(4-amino-3-methoxyphenoxy)-N-methylpropanamide (26 mg, 0.12 mmol) and 4-methylbenzenesulfonic acid hydrate (22 mg, 0.12 mmol) in NMP (0.4 mL) was stirred at 150° C. for 2.5 hours. The reaction mixture was diluted with methylene chloride and poured into an aqueous solution of K2CO3. The aqueous layer was extracted with methylene chloride and the combined organic extracts were dried over MgSO4 and evaporated. The crude product was purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford an impure sample of the desired product. The later was purified by flash chromatography on silica gel eluting with 0 to 3% methanol in methylene chloride to afford (2S)-2-(4-(5-chloro-4-(8-fluoroimidazo[1,2-a]pyridin-3-yl)pyrimidin-2-ylamino)-3-methoxyphenoxy)-N-methylpropanamide (9 mg, 16% yield); NMR spectrum: 1.46 (d, 3H), 2.64 (d, 3H), 3.75 (s, 3H), 4.78 (q, 1H), 6.53 (dd, 1H), 6.77 (d, 1H), 6.90 (bs, 1H), 7.34 (d, 1H), 7.42 (dd, 1H), 8.08 (q, 1H), 8.52 (s, 1H), 8.73 (s, 1H), 8.94 (s, 1H), 9.35 (bs, 1H); Mass spectrum: 471 (MH+).

The (S)-2-(4-amino-3-methoxyphenoxy)-N-methylpropanamide used as a starting material was prepared as described for 2-(4-amino-3-methoxyphenoxy)-N,N-dimethylacetamide (see Example 217, starting material) using (R)-methyl 2-hydroxypropanoate instead of methyl 2-hydroxyacetate in the first step and methanamine hydrochloride instead of dimethylamine hydrochloride in the third step; NMR spectrum: (CDCl₃) 1.53 (d, 3H), 2.58 (bs, 2H), 2.84 (d, 3H), 3.82 (s, 3H), 4.56 (q, 1H), 6.32 (dd, 1H), 6.44 (d, 1H), 6.52 (bs, 1H), 6.62 (d, 1H).

### Example 219

### (2R)-2-[4-[[5-chloro-4-(8-fluoroimidazo[1,2-a]pyridin-3-yl)pyrimidin-2-yl]amino]-3-methoxy-phenoxy]-N-methyl-propanamide

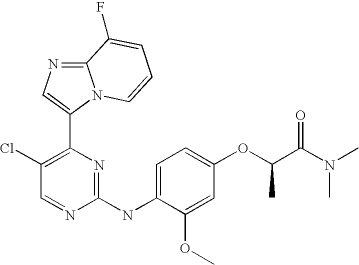

The title compound was prepared according to the procedure described in Example 218, starting from (S)-methyl 2-hydroxypropanoate instead of (R)-methyl 2-hydroxypropanoate in the first step.

### Example 220

### N-[2-[4-[[5-chloro-4-(8-fluoroimidazo[1,2-a]pyridin-3-yl)pyrimidin-2-yl]amino]-3-methoxy-N-methyl-anilino]ethyl]-2-hydroxy-N-methyl-acetamide

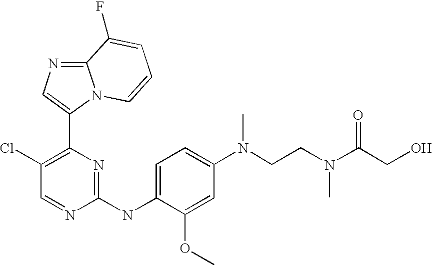

A mixture of 3-(2,5-dichloropyrimidin-4-yl)-8-fluoroimidazo[1,2-a]pyridine (Method 34, 150 mg, 0.51 mmol), N-(2-((4-amino-3-methoxyphenyl)(methyl)amino)ethyl)-2-hydroxy-N-methylacetamide (143 mg, 0.53 mmol) and para-toluenesulfonic acid (106 mg, 0.56 mmol) in 2-pentanol (4 mL) was heated at 120° C. for 2 hours. The reaction mixture was neutralised with water and K2CO3. The aqueous layer was extracted with methylene chloride and the organic phase dried over magnesium sulfate and concentrated. The crude product was purified by flash chromatography on silica gel eluting with 0 to 10% methanol in ethyl acetate to afford N-(2-((4-(5-chloro-4-(8-fluoroimidazo[1,2-a]pyridin-3-yl)pyrimidin-2-ylamino)-3-methoxyphenyl)(methyl)amino)ethyl)-2-hydroxy-N-methylacetamide (38 mg, 14%); NMR spectrum: (323° K) 2.91 (s, 1.95H), 2.92 (s, 1.05H), 2.95 (s, 1.05H), 2.97 (s, 1.95H), 3.38-3.60 (m, 4H), 3.77 (s, 3H), 4.02 (d, 2H), 4.32 (s, 0.65H), 4.38 (t, 0.35H), 6.30-6.38 (m, 1H), 6.43 (s, 0.35H), 6.53 (s, 0.65H), 6.78-6.85 (m, 1H), 7.20 (d, 0.65H), 7.24 (d, 0.35H), 7.35 (dd, 1H), 8.45 (s, 1H), 8.62 (s, 1H), 8.69 (s, 1H), 9.36 (bs, 1H); Mass spectrum: 514 (MH+).

The N-(2-((4-amino-3-methoxyphenyl)(methyl)amino)ethyl)-2-hydroxy-N-methylacetamide used as a starting material was prepared as follows:

A mixture of 4-fluoro-2-methoxy-1-nitrobenzene (1 g, 5.84 mmol), N1,N2-dimethylethane-1,2-diamine (0.763 mL, 5.84 mmol) and sodium hydride (0.280 g, 11.69 mmol) in DMA (4 mL) was stirred at 25° C. for 2 hours. The reaction mixture was quenched with a saturated aqueous solution of sodium bicarbonate and extracted with methylene chloride. The combined organic extracts were dried over magnesium sulfate and concentrated to afford the crude product which was purified by flash chromatography on silica gel eluting with 0 to 5% methanolic ammonia (7 N) in methylene chloride to afford N1-(3-methoxy-4-nitrophenyl)-N1,N2-dimethylethane-1,2-diamine (0.805 g, 57%) as a yellow oil.

2-(3H-[1,2,3]triazolo[4,5-b]pyridin-3-yl)-1,1,3,3-tetramethylisouronium hexafluorophosphate(V) (1.66 g, 4.37 mmol) was added to a stirred solution of N1-(3-methoxy-4-nitrophenyl)-N1,N2-dimethylethane-1,2-diamine (805 mg, 3.36 mmol), 2-hydroxyacetic acid (384 mg, 5.05 mmol) and N-ethyl-N-isopropylpropan-2-amine (0.880 mL, 5.05 mmol) in methylene chloride (20 mL) at 25° C. under nitrogen. The resulting solution was stirred for 16 hours and the reaction mixture was quenched with a saturated aqueous solution of ammonium chloride and extracted with methylene chloride. The combined organic phases were dried over magnesium sulfate and concentrated to afford a yellow solid which was purified by flash chromatography on silica gel eluting with 0 to 10% methanol in methylene chloride to afford 2-hydroxy-N-(2-((3-methoxy-4-nitrophenyl)-(methyl)amino)ethyl)-N-methylacetamide (780 mg, 78%).

A suspension of 2-hydroxy-N-(2-((3-methoxy-4-nitrophenyl)(methyl)amino)ethyl)-N-methylacetamide (200 mg, 0.67 mmol), platinum(IV) oxide (50 mg, 0.22 mmol) in EtOAc (5 mL)/ethanol (5 mL), was hydrogenated under 50 psi at 25° C. for 1 hours. The resulting suspension was filtered and the filtrate was concentrated to dryness. The crude product was purified by flash chromatography on silica gel eluting with 0 to 10% methanol in methylene chloride to afford N-(2-((4-amino-3-methoxyphenyl)(methyl)amino)ethyl)-2-hydroxy-N-methylacetamide (150 mg, 83%). NMR Spectrum: (DMSO at 232K) 2.77 (s, 1.2H), 2.78 (s, 1.8H), 2.85 (s, 1.8H), 2.88 (s, 1.2H), 3.25-3.37 (m, 3H), 3.39-3.45 (m, 2H), 3.75 (s, 3H), 3.92-4.05 (m, 4H), 4.23-4.30 (m, 1H), 6.12-6.19 (m, 1H), 6.31 (s, 0.4H), 6.38 (s, 0.6H), 6.50-6.57 (m, 1H)

### Example 221

### 2-[2-[4-[[5-chloro-4-(8-fluoroimidazo[1,2-a]pyridin-3-yl)pyrimidin-2-yl]amino]-3-methoxy-N-methyl-anilino]ethyl-methyl-amino]ethanol

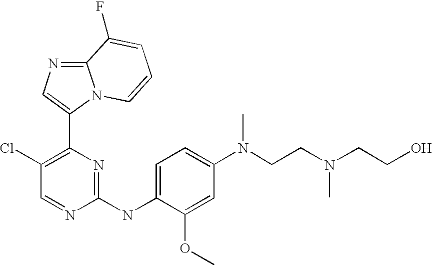

A mixture of 3-(2,5-dichloropyrimidin-4-yl)-8-fluoroimidazo[1,2-a]pyridine (Method 34, 200 mg, 0.68 mmol), 2-((2-((4-amino-3-methoxyphenyl)-(methyl)amino)ethyl)(methyl)amino)ethanol (210 mg, 0.75 mmol) and para-toluenesulfonic acid (258 mg, 1.36 mmol) in 2-pentanol (4 mL) was heated to 140° C. for 2 hours. The reaction mixture was quenched with water and K2CO3 was added. The aqueous layer was extracted with methylene chloride. The organic phase was concentrated to afford the crude product which was purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford 2-((2-((4-(5-chloro-4-(8-fluoroimidazo[1,2-a]pyridin-3-yl)pyrimidin-2-ylamino)-3-methoxyphenyl)(methyl)amino)ethyl)(methyl)amino)ethanol (186 mg, 55%); NMR spectrum: 2.27 (s, 3H), 2.47 (t, 2H), 2.55 (t, 2H), 2.98 (s, 3H), 3.44-3.51 (m, 4H), 3.75 (s, 3H), 4.37 (t, 1H), 6.30 (dd, 1H), 6.42 (d, 1H), 6.78 (bs, 1H), 7.16 (d, 1H), 7.40 (dd, 1H), 8.48 (s, 1H), 8.72 (s, 1H), 8.79 (s, 1H), 9.31 (bs, 1H); Mass spectrum: 500 (MH+).

The 2-((2-((4-amino-3-methoxyphenyl)(methyl)amino)ethyl)(methyl)amino)ethanol used as a starting material was prepared as follows:

Borane-methyl sulfide complex (7.33 ml, 14.6 mmol) was added to a stirred solution of 2-hydroxy-N-(2-((3-methoxy-4-nitrophenyl)(methyl)amino)ethyl)-N-methylacetamide (see Example 220, starting material, 436 mg, 1.47 mmol) in THF at 60° C. under nitrogen. The resulting solution was stirred at 60° C. for 15 minutes and RT for 1 hour. The volatiles were evaporated and the residue was dissolved in methylene chloride (20 mL) and washed with 1N NaOH (20 mL). The organic phase was dried over magnesium sulfate and concentrated to afford the crude product which was purified by flash chromatography on silica gel eluting with 0 to 5% methanol in ethyl acetate to afford 2-((2-((3-methoxy-4-nitrophenyl)(methyl)amino)ethyl)(methyl)amino)ethanol (280 mg, 67%).

A suspension of 2-((2-((3-methoxy-4-nitrophenyl)(methyl)amino)ethyl)(methyl)amino)ethanol (280 mg, 0.99 mmol), platinum(IV) oxide (100 mg, 0.44 mmol) in EtOAc (5 mL)/ethanol (5 mL) was hydrogenated under 50 psi at 25° C. for 2 hours. The resulting suspension was filtered and the filtrate was concentrated to dryness to afford 2-((2-((4-amino-3-methoxyphenyl)(methyl)amino)ethyl)(methyl)amino)ethanol (210 mg, 84%). NMR Spectrum: 2.22 (s, 3H), 2.39-2.4 (m, 4H), 2.77 (s, 3H), 3.20-3.28 (m, 2H), 3.40-3.48 (m, 2H), 3.74 (s, 3H), 4.05 (bs, 2H), 4.33 (bs, 1H), 6.13 (dd, 1H), 6.32 (d, 1H), 6.52 (d, 1H).

### Example 222

### 1-[4-[[5-chloro-4-(8-fluoroimidazo[1,2-a]pyridin-3-yl)pyrimidin-2-yl]amino]-3-methoxy-phenyl]-N-methyl-piperidine-4-carboxamide

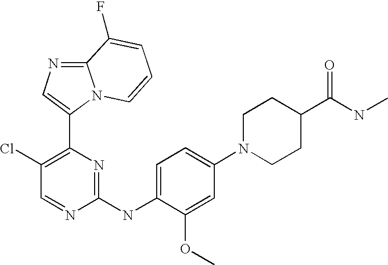

To a solution of 1-(4-(5-chloro-4-(8-fluoroimidazo[1,2-a]pyridin-3-yl)pyrimidin-2-ylamino)-3-methoxyphenyl)piperidine-4-carboxylic acid (150 mg, 0.27 mmol), 2-(3H-[1,2,3]triazolo[4,5-b]pyridin-3-yl)-1,1,3,3-tetramethylisouronium hexafluorophosphate(V) (134 mg, 0.35 mmol) and N-ethyl-N-isopropylpropan-2-amine (0.189 mL, 1.09 mmol) in DMF (2.5 mL) at 25° C., was added methanamine hydrochloride (22 mg, 0.33 mmol). The resulting mixture was stirred at 25° C. for 2 hours. The reaction mixture was then purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford 1-(4-(5-chloro-4-(8-fluoroimidazo[1,2-a]pyridin-3-yl)pyrimidin-2-ylamino)-3-methoxyphenyl)-N-methylpiperidine-4-carboxamide (95 mg, 68%) as a yellow solid; NMR spectrum: 1.61 (dd, 1H), 1.66 (dd, 1H), 1.66-1.74 (m, 2H), 2.17-2.26 (m, 1H), 3.53 (d, 3H), 2.60-2.69 (m, 2H), 3.68 (s, 3H), 3.68-3.75 (m, 2H), 6.46 (dd, 1H), 6.62 (d, 1H), 6.79 (bs, 1H), 7.17 (d, 1H), 7.34 (dd, 1H), 7.70 (q, 1H), 8.42 (s, 1H), 8.64 (s, 1H), 8.77 (s, 1H), 9.30 (bs, 1H); Mass spectrum: 510 (MH+).

The 1-(4-(5-chloro-4-(8-fluoroimidazo[1,2-a]pyridin-3-yl)pyrimidin-2-ylamino)-3-methoxyphenyl)piperidine-4-carboxylic acid used as a starting material was prepared as follows:

Methyl 1-(4-amino-3-methoxyphenyl)piperidine-4-carboxylate was obtained from methyl piperidine-4-carboxylate using the 2 steps procedure described in Method 7.

A mixture of 3-(2,5-dichloropyrimidin-4-yl)-8-fluoroimidazo[1,2-a]pyridine (Method 34, 300 mg, 1.02 mmol), methyl 1-(4-amino-3-methoxyphenyl)piperidine-4-carboxylate (269 mg, 1.02 mmol) and para-toluenesulfonic acid (232 mg, 1.22 mmol) in 2-pentanol (5 mL) was heated to 140° C. into a microwave reactor for 2 hours. The reaction mixture was diluted with ethyl acetate and washed with a saturated solution of sodium bicarbonate, then brine. The organic phase was dried (MgSO4), filtered and evaporated to dryness. The crude product was purified by flash chromatography on silica gel eluting with 0 to 50% EtOAc/petroleum ether to afford pentan-2-yl 1-(4-(5-chloro-4-(8-fluoroimidazo[1,2-a]pyridin-3-yl)pyrimidin-2-ylamino)-3-methoxyphenyl)piperidine-4-carboxylate (330 mg, 56%). Sodium hydroxide (10N in water, 0.58 mL, 5.8 mmol) was added to a stirred solution of pentan-2-yl 1-(4-(5-chloro-4-(8-fluoroimidazo[1,2-a]pyridin-3-yl)pyrimidin-2-ylamino)-3-methoxyphenyl)piperidine-4-carboxylate (330 mg, 0.58 mmol) in MeOH (10 mL). After 4 hours, the solvent was removed and the residue diluted with diethylether. The pH was adjusted to 2 with 6N hydrochloric acid and a yellow product precipitated. The solid was filtered, rinsed with H2O and dried over P2O5 under vacuum at 50° C. to provide 1-(4-(5-chloro-4-(8-fluoroimidazo[1,2-a]pyridin-3-yl)pyrimidin-2-ylamino)-3-methoxyphenyl)piperidine-4-carboxylic acid (285 mg, 99%). NMR spectrum: 2.03-2.16 (m, 2H), 2.16-2.26 (m, 2H), 2.67-2.78 (m, 1H), 3.56-3.67 (m, 2H), 3.67-3.76 (m, 2H), 3.89 (s, 3H), 7.28 (dd, 1H), 7.37 (ddd, 1H), 7.49 (d, 1H), 7.79 (dd, 1H), 8.01 (d, 1H), 8.65 (s, 1H), 9.03 (s, 1H), 9.65 (bs, 1H).

### Example 223

### 2-[4-[[5-chloro-4-(8-fluoroimidazo[1,2-a]pyridin-3-yl)pyrimidin-2-yl]amino]-N-(2-hydroxyethyl)-3-methoxy-anilino]ethanol

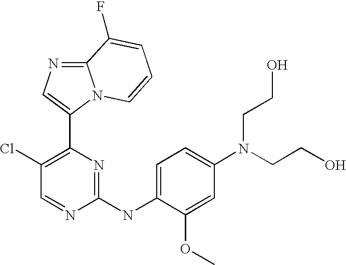

A mixture of 2-[4-amino-N-(2-hydroxyethyl)-3-methoxy-anilino]ethanol (0.120 g, 0.53 mmol), 4-methylbenzenesulfonic acid hydrate (0.101 g, 0.53 mmol) and 3-(2,5-dichloropyrimidin-4-yl)-8-fluoroimidazo[1,2-a]pyridine (0.15 g, 0.53 mmol) in 2-pentanol (1.5 ml) was sealed into a microwave tube. The reaction was heated to 140° C. for 2 hours in the microwave reactor. The mixture was diluted with water, basified with solid NaHCO₃ and the mixture was extracted three times with methylene chloride. The organic extracts were dried over MgSO4, filtered and concentrated. The crude product was purified by flash chromatography on silica gel eluting with 0 to 10% methanol in a 1:1 mixture of methylene chloride and ethyl acetate. The solvent was evaporated to dryness to afford 2,2′-(4-(5-chloro-4-(8-fluoroimidazo[1,2-a]pyridin-3-yl)pyrimidin-2-ylamino)-3-methoxyphenylazanediyl)diethanol (0.145 g, 58%); NMR spectrum: 3.45-3.52 (m, 4H), 3.56-3.66 (m, 4H), 3.73 (s, 3H), 4.79 (t, 2H), 6.30 (dd, 1H), 6.42 (d, 1H), 6.81 (bs, 1H), 7.11 (d, 1H), 7.39 (dd, 1H), 8.47 (s, 1H), 8.72 (s, 1H), 8.79 (s, 1H), 9.29 (bs, 1H); Mass spectrum: 473 (MH+).

The 2-[4-amino-N-(2-hydroxyethyl)-3-methoxy-anilino]ethanol used as a starting material was prepared from 2-(2-hydroxyethylamino)ethanol using the 2 steps procedure described in Method 7. NMR spectrum: 3.25 (t, 4H), 3.44-3.51 (m, 4H), 3.72 (s, 3H), 3.97 (bs, 2H), 4.62 (t, 2H), 6.12 (dd, 1H), 6.29 (d, 1H), 6.49 (d, 1H).

### Example 224

### 1-[4-[[5-chloro-4-(8-fluoroimidazo[1,2-a]pyridin-3-yl)pyrimidin-2-yl]amino]-3-methoxy-phenyl]piperazin-2-one

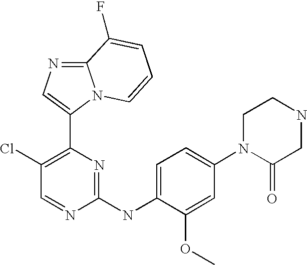

A mixture of tert-butyl 4-(4-amino-3-methoxyphenyl)-3-oxopiperazine-1-carboxylate (110 mg, 0.34 mmol), 3-(2,5-dichloropyrimidin-4-yl)-8-fluoroimidazo[1,2-a]pyridine (Method 34, 97 mg, 0.34 mmol) and 4-methylbenzenesulfonic acid hydrate (130 mg, 0.68 mmol) in NMP (2.0 mL) was stirred at 140° C. for 6 hours under inert atmosphere. The reaction mixture was diluted with ethyl acetate and washed with a 2M aqueous solution of sodium carbonate. After solvent removal, the crude product was purified by chromatography on silica gel eluting with 0 to 15% MeOH in methylene chloride to afford 1-(4-(5-chloro-4-(8-fluoroimidazo[1,2-a]pyridin-3-yl)pyrimidin-2-ylamino)-3-methoxyphenyl)piperazin-2-one (15 mg, 9%); NMR spectrum: 2.79 (bs, 1H), 3.04 (t, 2H), 3.42 (s, 2H), 3.64 (t, 2H), 3.76 (s, 3H), 6.91 (dd, 1H), 7.07 (ddd, 1H), 7.11 (d, 1H), 7.42 (dd, 1H), 7.55 (d, 1H), 8.56 (s, 1H), 8.76 (s, 1H), 9.06 (s, 1H), 9.42 (bs, 1H); Mass spectrum: 468 (MH+).

The tert-butyl 4-(4-amino-3-methoxyphenyl)-3-oxopiperazine-1-carboxylate used as a starting material was prepared as follows:

A mixture of tert-butyl 3-oxopiperazine-1-carboxylate (100 mg, 0.50 mmol), 4-chloro-2-methoxy-1-nitrobenzene (94 mg, 0.50 mmol), cesium carbonate (244 mg, 0.75 mmol), (9,9-dimethyl-9H-xanthene-4,5-diyl)bis(diphenylphosphine) (17.3 mg, 0.03 mmol) and diacetoxypalladium (4.5 mg, 0.02 mmol) was dried for 15 min under vacuum. Degassed dioxane (1.2 ml) was added and the resulting mixture was stirred at 90° C. for 16 hours under inert atmosphere. The reaction mixture was diluted with ethyl acetate and washed with a 2M aqueous solution of sodium carbonate. The crude material was purified on silica gel eluting with 20 to 60% ethyl acetate in petroleum ether to provide tert-butyl 4-(3-methoxy-4-nitrophenyl)-3-oxopiperazine-1-carboxylate (120 mg, 68%).

A solution of tert-butyl 4-(3-methoxy-4-nitrophenyl)-3-oxopiperazine-1-carboxylate (240 mg, 0.68 mmol) and platinum(IV) oxide (25 mg, 0.11 mmol) in ethanol (15 ml) was hydrogenated under 1 atm at 25° C. for 1 hour. The resulting solution was filtered and the filtrate was concentrated to dryness to afford tert-butyl 4-(4-amino-3-methoxyphenyl)-3-oxopiperazine-1-carboxylate (220 mg, 100%); NMR Spectrum: 1.44 (s, 9H), 3.57-3.68 (m, 4H), 3.33 (s, 3H), 4.01 (bs, 2H), 4.75 (bs, 2H), 6.56-6.61 (m, 2H), 6.74 (s, 1H)

### Example 225

### 2-[4-[[5-chloro-4-(8-fluoroimidazo[1,2-a]pyridin-3-yl)pyrimidin-2-yl]amino]-3-methoxy-N-methyl-anilino]-N,N-dimethyl-acetamide

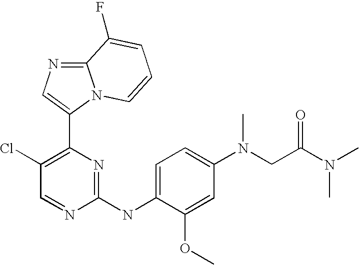

2-(3H-[1,2,3]triazolo[4,5-b]pyridin-3-yl)-1,1,3,3-tetramethylisouronium hexafluorophosphate(V) (83 mg, 0.22 mmol) was added to a stirred solution of sodium 2-((4-(5-chloro-4-(8-fluoroimidazo[1,2-a]pyridin-3-yl)pyrimidin-2-ylamino)-3-methoxyphenyl)(methyl)amino)acetate (70 mg, 0.15 mmol), dimethylamine hydrochloride (14.30 mg, 0.18 mmol) and N-ethyl-N-isopropylpropan-2-amine (0.038 mL, 0.22 mmol) in DMF (0.7 mL) at room temperature. The resulting solution was stirred for 16 hours. The crude mixture was purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford 2-((4-(5-chloro-4-(8-fluoroimidazo[1,2-a]pyridin-3-yl)pyrimidin-2-ylamino)-3-methoxyphenyl)(methyl)amino)-N,N-dimethylacetamide (45 mg, 64%); NMR spectrum: 2.86 (s, 3H), 3.01 (s, 3H), 3.04 (s, 3H), 3.71 (s, 3H), 4.29 (s, 2H), 6.23 (dd, 1H), 6.8 (d, 1H), 6.94 (bs, 1H), 7.09 (d, 1H), 7.38 (dd, 1H), 8.46 (s, 1H), 8.74 (s, 1H), 8.78 (s, 1H), 9.27 (bs, 1H); Mass spectrum: 484 (MH+).

The sodium 2-((4-(5-chloro-4-(8-fluoroimidazo[1,2-a]pyridin-3-yl)pyrimidin-2-ylamino)-3-methoxyphenyl)(methyl)amino)acetate used as a starting material was prepared as follows:

4-fluoro-2-methoxy-1-nitrobenzene (2.5 g, 14.6 mmol) and triethylamine (4.48 ml, 32.1 mmol) were added to a stirred solution of tert-butyl 2-(methylamino)acetate hydrochloride (2.92 g, 16.1 mmol) in DMSO (7.5 ml). The resulting solution was heated at 125° C. for 12 hours. The reaction mixture was cooled mixed with water extracted with ethyl acetate. The combined organic extracts were washed with a 1M aqueous solution of sodium hydroxide, water and brine, dried over magnesium sulfate and concentrated. The crude product was triturated in diethylether and pentane to afford tert-butyl 2-((3-methoxy-4-nitrophenyl)(methyl)amino)acetate (2.20 g, 50%).

A suspension of tert-butyl 2-((3-methoxy-4-nitrophenyl)(methyl)amino)acetate (2.2 g, 7.42 mmol) and platinum(IV) oxide (0.118 g, 0.52 mmol) in ethyl acetate (10 ml) and ethanol (40 ml) was hydrogenated under 1.3 bar at 25° C. for 3 hours. The reaction mixture was filtered and the filtrate was concentrated to afford tert-butyl 2-((4-amino-3-methoxyphenyl)(methyl)amino)acetate (2.00 g, 100%) as a brown oil which solidified on standing.

A mixture of diacetoxypalladium (0.052 g, 0.23 mmol), potassium phosphate (2.43 g, 11.4 mmol), 2,2′-bis(diphenylphosphino)-1,1′-binaphthyl (0.286 g, 0.46 mmol), 3-(2,5-dichloropyrimidin-4-yl)-8-fluoroimidazo[1,2-a]pyridine (Method 34, 1.3 g, 4.59 mmol) and tert-butyl 2-((4-amino-3-methoxyphenyl)(methyl)amino)acetate (1.223 g, 4.59 mmol) in dioxane (20 ml) was heated at 100° C. under an inert atmosphere for 1 h30. After cooling, the mixture was filtered through Celite and the filtrate was evaporated. The crude product was purified by flash chromatography on silica gel eluting with 0 to 100% EtOAc in CH₂Cl₂ to afford 1-(4-(3-methoxy-4-(5-methyl-4-(1-(phenylsulfonyl)-1H-indol-3-yl)pyrimidin-2-ylamino)phenyl)piperazin-1-yl)ethanone (1.76 g, 56%) as a yellow oil.

Sodium hydroxide (2N in water, 1.07 mL, 2.13 mmol) was added to a stirred suspension of tert-butyl 2-((4-(5-chloro-4-(8-fluoroimidazo[1,2-a]pyridin-3-yl)pyrimidin-2-ylamino)-3-methoxyphenyl)(methyl)amino)acetate (365 mg, 0.71 mmol) in EtOH (7 mL). The resulting suspension was stirred at 60° C. for 4 hours. The reaction mixture was concentrated to dryness to give the desired sodium 2-((4-(5-chloro-4-(8-fluoroimidazo[1,2-a]pyridin-3-yl)pyrimidin-2-ylamino)-3-methoxyphenyl)(methyl)amino)acetate (403 mg, quant. yield) which was used without further purification in the next step.

### Example 226

### 2-[4-[[5-chloro-4-(8-fluoroimidazo[1,2-a]pyridin-3-yl)pyrimidin-2-yl]amino]-3-methoxy-N-methyl-anilino]-N-methyl-acetamide

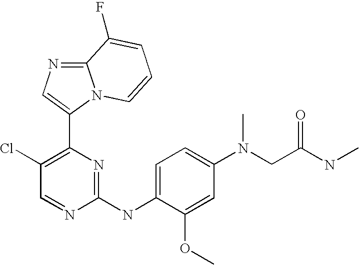

The title compound was prepared according to the procedure described in Example 225, starting from methanamine hydrochloride instead of dimethylamine hydrochloride; NMR spectrum: 2.62 (d, 3H), 3.06 (s, 3H), 3.72 (s, 3H), 3.93 (s, 2H), 6.22 (dd, 1H), 6.37 (d, 1H), 6.86 (bs, 1H), 7.16 (d, 1H), 7.39 (dd, 1H), 7.87 (q, 1H), 8.47 (s, 1H), 8.71 (s, 1H), 8.81 (s, 1H), 9.30 (bs, 1H); Mass spectrum: 470 (MH+).

### Example 227

### 2-[4-[[5-chloro-4-(8-fluoroimidazo[1,2-a]pyridin-3-yl)pyrimidin-2-yl]amino]-3-methoxy-N-methyl-anilino]-1-piperazin-1-yl-ethanone

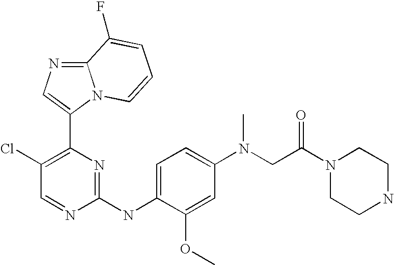

2-(3H-[1,2,3]triazolo[4,5-b]pyridin-3-yl)-1,1,3,3-tetramethylisouronium hexafluorophosphate(V) (158 mg, 0.42 mmol) was added to a stirred solution of sodium 2-((4-(5-chloro-4-(8-fluoroimidazo[1,2-a]pyridin-3-yl)pyrimidin-2-ylamino)-3-methoxyphenyl)(methyl)amino)acetate (see Example 225, starting material, 133 mg, 0.28 mmol), tert-butyl piperazine-1-carboxylate (62 mg, 0.33 mmol) and N-ethyl-N-isopropylpropan-2-amine (0.073 mL, 0.42 mmol) in DMF (1.5 mL) at room temperature. The resulting solution was stirred for 16 hours then the reaction mixture was partitioned between aq. sat. NaHCO3 and methylene chloride. The organic layer was dried over MgSO4 and concentrated. The residue was dissolved in methylene chloride (1 mL), TFA (1 mL) was added and the reaction was stirred at room temperature for 2 hours. The mixture was concentrated to dryness and the residue was dissolved in methylene chloride, basified with NH3 7N in methanol and concentrated to dryness. The crude product was purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford the desired product which was further purified by flash chromatography on silica gel eluting with 0 to 7% methanolic ammonia (7 N) in dichloromethane (35 mg, 24%); NMR spectrum: 2.65-2.72 (m, 2H), 2.73-2.81 (m, 2H), 3.02 (s, 3H), 3.39-3.49 (m, 4H), 3.71 (s, 3H), 4.31 (s, 2H), 6.24 (dd, 1H), 6.36 (d, 1H), 6.93 (bs, 1H), 7.11 (d, 1H), 7.38 (dd, 1H), 8.46 (s, 1H), 8.74 (s, 1H), 8.78 (s, 1H), 9.27 (bs, 1H); Mass spectrum: 525 (MH+).

### Example 228

### 2-[4-[4-[(5-fluoro-4-imidazo[1,2-a]pyridin-3-yl-pyrimidin-2-yl)amino]-3-methoxy-phenyl]piperazin-1-yl]-2-methyl-propan-1-ol

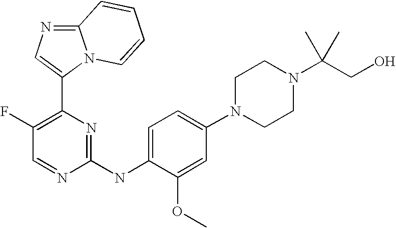

3-(2-chloro-5-fluoropyrimidin-4-yl)imidazo[1,2-a]pyridine (Method 2, 130 mg, 0.52 mmol), 2-(4-(4-amino-3-methoxyphenyl)piperazin-1-yl)-2-methylpropan-1-ol (146 mg, 0.52 mmol) and 4-methylbenzenesulfonic acid hydrate (199 mg, 1.05 mmol) in 2-pentanol (1.2 ml) were stirred at 140° C. overnight. The 2-pentanol was evaporated, the mixture was dissolved in the minimum of DMF with 2 drops of 30% aqueous ammonia, filtered and purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford 2-(4-(4-(5-fluoro-4-(imidazo[1,2-a]pyridin-3-yl)pyrimidin-2-ylamino)-3-methoxyphenyl)piperazin-1-yl)-2-methylpropan-1-ol (140 mg, 54%); NMR spectrum: 1.00 (s, 6H), 2.70-2.77 (m, 4H), 3.12-3.21 (m, 4H), 3.34 (d partially hidden by H2O, 2H), 3.75 (s, 3H), 4.31 (t, 1H), 6.52 (dd, 1H), 6.67 (d, 1H), 6.93 (dd, 1H), 7.32 (d, 1H), 7.52 (ddd, 1H), 7.78 (d, 1H), 8.36 (d, 1H), 8.43 (d, 1H), 8.59 (s, 1H), 9.76 (bs, 1H); Mass spectrum: 492 (MH+).

The 2-(4-(4-amino-3-methoxyphenyl)piperazin-1-yl)-2-methylpropan-1-ol used as a starting material was prepared as follows:

A mixture of 2-methyl-2-(piperazin-1-yl)propan-1-01 (Bioorganic & Medicinal Chemistry Letters 2007, p. 5330, 0.971 g, 6.14 mmol), N,N-diisopropylethylamine (1.22 mL, 7.01 mmol) and 4-fluoro-2-methoxy-1-nitrobenzene (1 g, 5.84 mmol) in DMA (5 mL) was heated at 90° C. for 16 hours. After cooling water was added, the precipitate was collected, washed with water and dried over P2O5 to afford 2-(4-(3-methoxy-4-nitrophenyl)piperazin-1-yl)-2-methylpropan-1-ol (0.964 g, 53%) as a yellow solid.

A suspension of 2-(4-(3-methoxy-4-nitrophenyl)piperazin-1-yl)-2-methylpropan-1-ol (940 mg, 3.04 mmol) and platinum(IV) oxide (34.5 mg, 0.15 mmol) in ethanol (100 ml) was hydrogenated under 1.3 bars at 25° C. for 2 hours. The suspension was filtered, the filtrate was concentrated and the residue purified by flash chromatography on silica gel eluting with 3 to 10% methanol in methylene chloride to afford 2-(4-(4-amino-3-methoxyphenyl)piperazin-1-yl)-2-methylpropan-1-ol (743 mg, 88%); NMR spectrum: 0.96 (s, 6H), 2.62-2.70 (m, 4H), 2.87-2.95 (m, 4H), 3.29 (d, 2H), 3.73 (s, 3H), 4.18 (bs, 2H), 4.23 (t, 1H), 6.26 (dd, 1H), 6.46 (d, 1H), 6.51 (d, 1H).

### Example 229

### 2-[4-[4-[(4-imidazo[1,2-a]pyridin-3-yl-5-methyl-pyrimidin-2-yl)amino]-3-methoxy-phenyl]piperazin-1-yl]-2-methyl-propan-1-ol

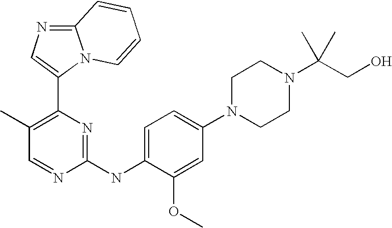

The title compound was prepared according to the procedure described in Example 228, starting from 3-(2-chloro-5-methylpyrimidin-4-yl)imidazo[1,2-a]pyridine (Method 5) instead of 3-(2-chloro-5-fluoropyrimidin-4-yl)imidazo[1,2-a]pyridine; NMR spectrum: 0.99 (s, 6H), 2.36 (s, 3H), 2.66-2.76 (m, 4H), 3.08-3.18 (m, 4H), 3.32 (d partially hidden by H2O, 2H), 3.77 (s, 3H), 4.30 (t, 1H), 6.49 (dd, 1H), 6.66 (d, 1H), 6.88 (dd, 1H), 7.39 (d, 1H), 7.43 (ddd, 1H), 7.71 (d, 1H), 8.20 (d, 1H), 8.25 (d, 1H), 8.29 (s, 1H), 9.59 (s, 0.5H), 9.61 (s, 0.5H); Mass spectrum: 488 (MH+).

### Example 230

### 2-[4-[4-[(5-chloro-4-pyrazolo[1,5-a]pyridin-3-yl-pyrimidin-2-yl)amino]-3-methoxy-phenyl]piperazin-1-yl]-2-methyl-propan-1-ol

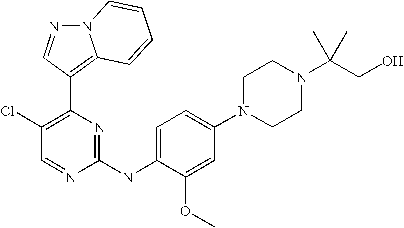

The title compound was prepared according to the procedure described in Example 228, starting from 3-(2,5-dichloropyrimidin-4-yl)pyrazolo[1,5-a]pyridine (Method 35) instead of 3-(2-chloro-5-fluoropyrimidin-4-yl)imidazo[1,2-a]pyridine; NMR spectrum: 1.00 (s, 6H), 2.69-2.76 (m, 4H), 3.11-3.20 (m, 4H), 3.31 (d partially hidden by H2O, 2H), 3.74 (s, 3H), 4.30 (t, 1H), 6.51 (dd, 1H), 6.65 d, 1H), 7.12 (dd, 1H), 7.28 (bs, 1H), 7.30 (d, 1H), 8.31 (bs, 1H), 8.34 (s, 1H), 8.51 (s, 1H), 8.82 (d, 1H), 8.94 (s, 1H); Mass spectrum: 508 (MH+).

### Example 231

### 2-[4-[3-methoxy-4-[(5-methyl-4-pyrazolo[1,5-a]pyridin-3-yl-pyrimidin-2-yl)amino]phenyl]piperazin-1-yl]-2-methyl-propan-1-ol

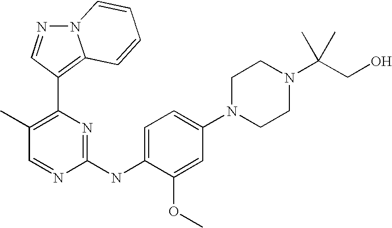

The title compound was prepared according to the procedure described in Example 228, starting from 3-(2-chloro-5-methyl-pyrimidin-4-yl)pyrazolo[1,5-a]pyridine (Method 44) instead of 3-(2-chloro-5-fluoropyrimidin-4-yl)imidazo[1,2-a]pyridine; NMR spectrum: 1.00 (s, 6H), 2.38 (s, 3H), 2.68-2.77 (m, 4H), 3.07-3.19 (m, 4H), 3.33 (bs partially hidden by H2O, 2H), 3.77 (s, 3H), 4.30 (t, 1H), 6.49 (d, 1H), 6.65 (s, 1H), 7.08 (dd, 1H), 7.30 (dd, 1H), 7.55 (d, 1H), 7.91 (s, 1H), 8.20 (s, 1H), 4.42 (d, 1H), 8.55 (s, 1H), 8.80 (d, 1H); Mass spectrum: 488 (MH+).

### Example 232

### 2-[7-[4-[(4-imidazo[1,2-a]pyridin-3-yl-5-methyl-pyrimidin-2-yl)amino]-3-methoxy-phenyl]-9-oxa-3,7-diazabicyclo[3.3.1]nonan-3-yl]ethanol

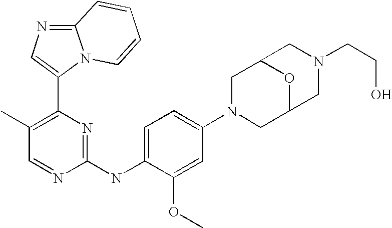

The title compound was prepared according to the procedure described in Example 144, starting from 3-(2-chloro-5-methylpyrimidin-4-yl)imidazo[1,2-a]pyridine (Method 5) instead of 3-(2,5-dichloropyrimidin-4-yl)imidazo[1,2-a]pyridine; NMR spectrum: 2.32 (t, 2H), 2.36 (s, 3H), 2.43-2.50 (m partially hidden by DMSOd5, 2H), 2.92-2.99 (m, 2H), 3.05-3.12 (m, 2H), 3.43-3.49 (m, 2H), 3.63-3.70 (m, 2H), 3.76 (s, 3H), 4.02-4.07 (m, 2H), 4.19 (t, 1H), 6.39 (dd, 1H), 6.52 (d, 1H), 6.85 (dd, 1H), 7.33 (d, 1H), 7.41 (dd, 1H), 7.70 (d, 1H), 8.19 (s, 1H), 8.25 (s, 1H), 8.27 (s, 1H), 9.58 (bs, 1H); Mass spectrum: 502 (MH+).

### Example 233

### 1-[4-[3-methoxy-4-[(5-methyl-4-pyrazolo[1,5-a]pyridin-3-yl-pyrimidin-2-yl)amino]phenyl]-1-piperidyl]ethanone

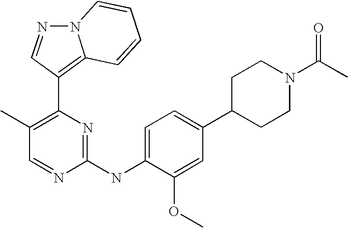

The title compound was prepared according to the procedure described in Example 3.21, starting from 3-(2-chloro-5-methyl-pyrimidin-4-yl)pyrazolo[1,5-a]pyridine (Method 44) instead of 3-(2,5-dichloropyrimidin-4-yl)imidazo[1,2-a]pyridine; NMR spectrum: 1.43-1.57 (m, 1H), 1.59-1.72 (m, 1H), 1.77-1.87 (m, 2H), 2.04 (s, 3H), 2.40 (s, 3H), 2.55-2.63 (m, 1H), 2.72-2.81 (m, 1H), 3.09-3.17 (m, 1H), 3.83 (s, 3H), 3.91-3.97 (m, 1H), 4.52-4.59 (m, 1H), 6.81 (dd, 1H), 6.96 (d, 1H), 7.09 (ddd, 1H), 7.36 (dd, 1H), 7.87 (d, 1H), 7.98 (s, 1H), 8.26 (s, 1H), 8.47 (d, 1H), 8.57 (s, 1H), 8.82 (d, 1H); Mass spectrum: 457 (MH+).

### Example 234

### 1-[4-[4-[(5-cyclopropyl-4-pyrazolo[1,5-a]pyridin-3-yl-pyrimidin-2-yl)amino]-3-methoxy-phenyl]piperazin-1-yl]ethanone

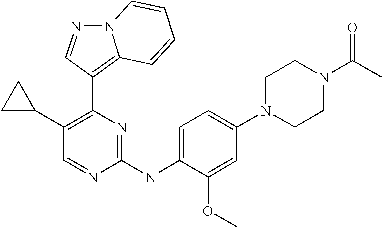

The title compound was prepared according to the procedure described in Example 128, using 3-(2-chloro-5-cyclopropyl-pyrimidin-4-yl)pyrazolo[1,5-a]pyridine instead of 3-(2,5-dichloropyrimidin-4-yl)pyrazolo[1,5-a]pyridine; NMR spectrum: 0.56-0.70 (m, 2H), 0.96-1.08 (m, 2H), 2.05 (bs, 1H), 2.07 (s, 3H), 3.07-3.23 (m, 4H), 4.24-4.58 (m, 4H), 3.79 (s, 3H), 6.55 (d, 1H), 6.72 (s, 1H), 7.08 (dd, 1H), 7.31 (dd, 1H), 7.57 (d, 1H), 8.01 (s, 1H), 8.18 (s, 1H), 8.80 (d, 1H), 8.86 (s, 1H); Mass spectrum: 484 (MH+).

The 3-(2-chloro-5-cyclopropyl-pyrimidin-4-yl)pyrazolo[1,5-a]pyridine used as a starting material was prepared as follows:

A mixture of (E)-4-(2-butoxyvinyl)-2-chloro-5-cyclopropylpyrimidine (see Example 201, starting material, 252 mg, 1.0 mmol), 1-aminopyridinium iodide (221 mg, 1.0 mmol) and potassium carbonate (345 mg, 2.49 mmol) in DMF (2.5 ml) was stirred at room temperature for 1 hour then heated at 80° C. for 3 hours. The reaction mixture was mixed with water, the precipitate was filtered and solubilised in methylene chloride. This solution was dried (MgSO4), filtered and evaporated. The crude product was purified by flash chromatography on silica gel eluting with 3% diethylether in methylene chloride to afford 3-(2-chloro-5-cyclopropylpyrimidin-4-yl)pyrazolo[1,5-a]pyridine (130 mg, 48%) as a dark orange solid; NMR spectrum: 0.75-0.84 (m, 2H), 1.11-1.20 (m, 2H), 2.15-2.24 (m, 1H), 7.22 (ddd, 1H), 7.67 (ddd, 1H), 8.49 (s, 1H), 8.61 (d, 1H), 8.94 (d, 1H), 8.98 (s, 1H).

### Example 235

### (2R)-2-amino-1-[4-[3-methoxy-4-[(5-methyl-4-pyrazolo[1,5-a]pyridin-3-yl-pyrimidin-2-yl)amino]phenyl]piperazin-1-yl]propan-1-one

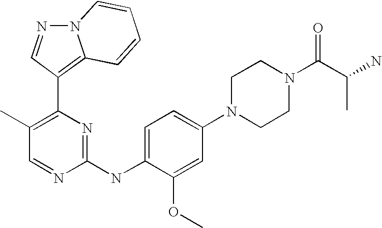

The title compound was prepared according to the procedure described in Example 24.14, using N-(2-methoxy-4-piperazin-1-yl-phenyl)-5-methyl-4-pyrazolo[1,5-a]pyridin-3-yl-pyrimidin-2-amine (Example 159) as the starting material; NMR spectrum: 1.11 (d, 3H), 2.39 (s, 3H), 3.08-3.31 m, 4H), 3.58-3.74 (m, 4H), 3.80 (s, 3H), 3.82 (q, 1H), 6.55 (dd, 1H), 6.73 (d, 1H), 7.08 (ddd, 1H), 7.32 (ddd, 1H), 7.63 (d, 1H), 7.93 (s, 1H), 8.21 (s, 1H), 8.42 (d, 1H), 8.56 (s, 1H), 8.80 (d, 1H); Mass spectrum: 487 (MH+).

### Example 236

### 2-(dimethylamino)-1-[4-[4-methoxy-3-[(5-methyl-4-pyrazolo[1,5-a]pyridin-3-yl-pyrimidin-2-yl)amino]phenyl]piperazin-1-yl]ethanone

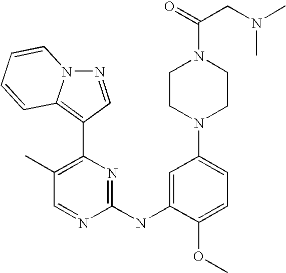

The title compound was prepared according to the procedure described in Example 152, using N-(2-methoxy-5-piperazin-1-yl-phenyl)-5-methyl-4-pyrazolo[1,5-a]pyridin-3-yl-pyrimidin-2-amine (Example 165) as the starting material; NMR spectrum: 2.17 (s, 6H), 2.41 (s, 3H), 2.85-3.00 (m, 4H), 3.08 (s, 2H), 3.44-3.53 (m, 2H), 3.53-3.64 (m, 2H), 3.81 (s, 3H), 6.61 (dd, 1H), 6.95 (d, 1H), 7.10 (dd, 1H), 7.41 (dd, 1H), 7.85 (d, 1H), 7.93 (s, 1H), 8.32 (s, 1H), 8.43 (d, 1H), 8.58 (s, 1H), 8.85 d, 1H); Mass spectrum: 501 (MH+).

### Example 237

### 1-[4-[3-methoxy-4-[(5-methyl-4-pyrazolo[1,5-a]pyridin-3-yl-pyrimidin-2-yl)amino]phenyl]-1-piperidyl]-2-(methylamino)ethanone

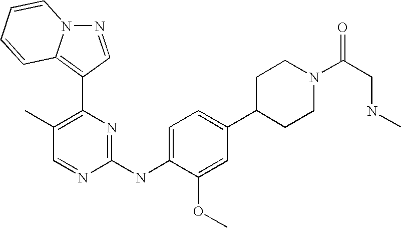

The title compound was prepared according to the procedure described in Example 140, using N-[2-methoxy-4-(4-piperidyl)phenyl]-5-methyl-4-pyrazolo[1,5-a]pyridin-3-yl-pyrimidin-2-amine (Example 162) as the starting material; NMR spectrum: 1.45-1.58 (m, 1H), 1.58-1.71 (m, 1H), 1.77-1.89 (m, 2H), 1.97 (bs, 1H), 2.29 (s, 3H), 2.40 (bs, 3H), 2.59-2.70 (m, 1H), 2.72-2.83 (m, 1H), 3.01-3.13 (m, 1H), 3.35 (s, 2H), 3.84 (s, 3H), 3.89-4.01 (m, 1H), 4.51-4.63 (m, 1H), 6.81 (dd, 1H), 6.95 (d, 1H), 7.09 (ddd, 1H), 7.36 (ddd, 1H), 7.87 (d, 1H), 7.98 (s, 1H), 8.26 (s, 1H), 8.47 (d, 1H), 8.57 (s, 1H), 8.82 (d, 1H); Mass spectrum: 486 (MH+).

### Example 238

### 1-[4-[4-[(5-fluoro-4-pyrazolo[1,5-a]pyridin-3-yl-pyrimidin-2-yl)amino]-3-methoxy-phenyl]-1-piperidyl]-2-(methylamino)ethanone

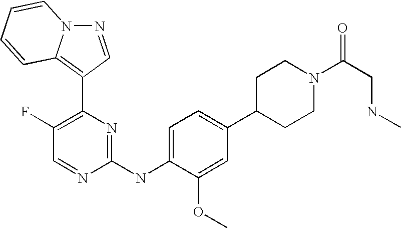

The title compound was prepared according to the procedure described in Example 140, using 5-fluoro-N-[2-methoxy-4-(4-piperidyl)phenyl]-4-pyrazolo[1,5-a]pyridin-3-yl-pyrimidin-2-amine (Example 173) as the starting material; NMR spectrum: 1.47-1.59 (m, 1H), 1.59-1.72 (m, 1H), 1.80-1.90 (m, 2H), 2.07 (bs, 1H), 2.31 (s, 3H), 2.60-2.72 (m, 1H), 2.76-2.86 (m, 1H), 3.04-3.14 (m, 1H), 3.37 (s, 2H), 3.81 (s, 3H), 3.90-4.01 (m, 1H), 4.53-4.63 (m, 1H), 6.85 (dd, 1H), 6.98 (s, 1H), 7.15 (ddd, 1H), 7.42 (dd, 1H), 7.68 (d, 1H), 8.39 (s, 1H), 8.41 (d, 1H), 8.51 (d, 1H), 8.57 (d, 1H), 8.87 (d, 1H); Mass spectrum: 490 (MH+).

### Example 239

### 2-[4-(1-acetyl-4-piperidyl)-2-methoxy-anilino]-4-pyrazolo[1,5-a]pyridin-3-yl-pyrimidine-5-carbonitrile

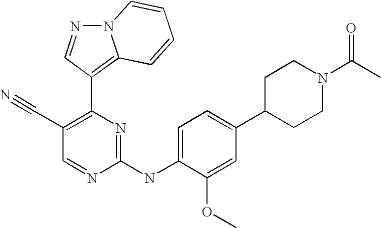

3-(5-bromo-2-chloro-pyrimidin-4-yl)pyrazolo[1,5-a]pyridine was prepared in 29% yield according to Method 35 using (E)-5-bromo-4-(2-butoxyvinyl)-2-chloropyrimidine as the starting material.

(E)-5-bromo-4-(2-butoxyvinyl)-2-chloropyrimidine was prepared in 39% yield according to Method 1 using 5-bromo-2,4-dichloropyrimidine as the starting material.

A mixture of 3-(5-bromo-2-chloro-pyrimidin-4-yl)pyrazolo[1,5-a]pyridine (150 mg, 0.48 mmol), 1-(4-(4-amino-3-methoxyphenyl)piperidin-1-yl)ethanone (124 mg, 0.48 mmol) and p-toluenesulfonic acid (92 mg, 0.48 mmol) in 2-pentanol (2.5 mL) was stirred at 150° C. for 2 hours into a microwave reactor. The reaction mixture was diluted with ethyl acetate and washed with a saturated aqueous solution of sodium bicarbonate, then brine and dried over MgSO4. This solution was evaporated and the residue dissolved in methylene chloride (5 ml) and acetic anhydride (1 ml). The solution was stirred for one hour at 50° C. then evaporated to dryness. The crude product was purified by flash chromatography on silica gel eluting with 0 to 6% MeOH(NH3 7N) in CH₂Cl₂ to afford 1-(4-(4-(5-bromo-4-(pyrazolo[1,5-a]pyridin-3-yl)pyrimidin-2-ylamino)-3-methoxyphenyl)piperidin-1-yl)ethanone (163 mg). This product was contaminated by around 20% of 5-bromo-N-[2-methoxy-4-(4-piperidyl)phenyl]-4-pyrazolo[1,5-a]pyridin-3-yl-pyrimidin-2-amine but was used in the next step without further purification.

A mixture of 1-(4-(4-(5-bromo-4-(pyrazolo[1,5-a]pyridin-3-yl)pyrimidin-2-ylamino)-3-methoxyphenyl)piperidin-1-yl)ethanone (80% pure, 160 mg, 0.25 mmol), dicyanozinc (54 mg, 0.46 mmol), zinc dust (2.0 mg, 0.03 mmol), tris(dibenzylideneacetone)dipalladium (7.0 mg, 7.6 μmol) and (9,9-dimethyl-9H-xanthene-4,5-diyl)bis(diphenylphosphine) (17.7 mg, 0.03 mmol) in DMA (1.6 ml) was stirred at 140° C. for 5 hours. After evaporation to dryness, the reaction mixture was purified by flash chromatography on silica gel eluting with 0 to 10% MeOH in EtOAc to give a solid which was further purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford 2-(4-(1-acetylpiperidin-4-yl)-2-methoxyphenylamino)-4-(pyrazolo[1,5-a]pyridin-3-yl)pyrimidine-5-carbonitrile (30 mg, 13% over 2 steps); NMR spectrum: (323° K) 1.45-1.62 (m, 1H), 1.62-1.75 (m, 1H), 1.79-1.94 (m, 2H), 2.04 (s, 3H), 2.59 (bs, 2H), 2.78-2.89 (m, 1H), 3.77 (s, 3H), 3.89-4.03 (m, 1H), 4.50-4.65 (m, 1H), 6.89 (d, 1H), 7.03 (s, 1H), 7.16 (dd, 1H), 7.36 (bs, 1H), 7.44 (d, 1H), 8.29 (bs, 1H), 8.70 (s, 1H), 8.84 (d, 1H), 8.92 (s, 1H), 9.31 (s, 1H); Mass spectrum: 468 (MH+).

### Example 240

### 2-[4-[1-(2-hydroxyacetyl)-4-piperidyl]-2-methoxy-anilino]-4-pyrazolo[1,5-a]pyridin-3-yl-pyrimidine-5-carbonitrile

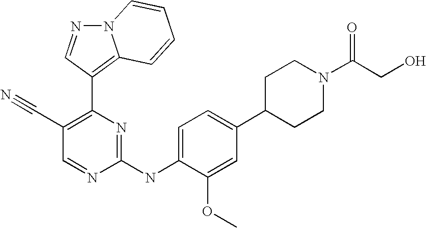

A mixture of 3-(5-bromo-2-chloropyrimidin-4-yl)pyrazolo[1,5-a]pyridine (see Example 239, starting material, 400 mg, 1.29 mmol), 1-(4-(4-amino-3-methoxyphenyl)piperidin-1-yl)ethanone (331 mg, 1.29 mmol) and p-toluenesulfonic acid (614 mg, 3.23 mmol) in 2-pentanol (8 mL) was stirred at 150° C. for 2 hours into a microwave reactor. The reaction mixture was diluted with ethyl acetate and washed with a saturated aqueous solution of sodium bicarbonate, then brine and dried over MgSO4. After evaporation of the solvent, the crude product was purified by flash chromatography on silica gel eluting with 0 to 12% MeOH(NH3 7N) in CH₂Cl₂ to afford a solid which was further purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent to afford 5-bromo-N-[2-methoxy-4-(4-piperidyl)phenyl]-4-pyrazolo[1,5-a]pyridin-3-yl-pyrimidin-2-amine (195 mg, 31%); NMR spectrum: 1.50-1.64 (m, 2H), 1.67-1.80 (m, 2H), 2.52 (bs partially hidden by DMOd5, 1H), 2.55-2.65 (m, 2H), 3.00-3.09 (m, 2H), 3.79 (s, 3H), 4.11 (bs, 1H), 6.82 (d, 1H), 6.96 (s, 1H), 7.13 (dd, 1H), 7.32 (dd, 1H), 7.54 (d, 1H), 8.31 (bs, 1H), 8.49 (s, 1H), 8.59 (s, 1H), 8.83 (d, 1H), 9.04 (s, 1H); Mass spectrum: 479 (MH+).

2-(3H-[1,2,3]triazolo[4,5-b]pyridin-3-yl)-1,1,3,3-tetramethylisouronium hexafluorophosphate(V) (170 mg, 0.45 mmol) was added to a solution of 5-bromo-N-(2-methoxy-4-(piperidin-4-yl)phenyl)-4-(pyrazolo[1,5-a]pyridin-3-yl)pyrimidin-2-amine (165 mg, 0.34 mmol), 2-hydroxyacetic acid (31.4 mg, 0.41 mmol) and N-ethyl-N-isopropylpropan-2-amine (0.180 mL, 1.03 mmol) in DMF (2.5 mL) at 25° C. The resulting slurry was stirred at 25° C. for 2 hours then DMF was evaporated and the residue was taken-up into methylene chloride with 10% MeOH and this solution was washed with water. The organic extract was dried (MgSO4), filtered and evaporation to dryness to give 1-[4-[4-[(5-bromo-4-pyrazolo[1,5-a]pyridin-3-yl-pyrimidin-2-yl)amino]-3-methoxy-phenyl]-1-piperidyl]-2-hydroxy-ethanone (190 mg) which was used in the next step without further purification. 2-[4-[1-(2-hydroxyacetyl)-4-piperidyl]-2-methoxy-anilino]-4-pyrazolo[1,5-a]pyridin-3-yl-pyrimidine-5-carbonitrile was prepared from 1-[4-[4-[(5-bromo-4-pyrazolo[1,5-a]pyridin-3-yl-pyrimidin-2-yl)amino]-3-methoxy-phenyl]-1-piperidyl]-2-hydroxy-ethanone using the procedure described in Example 239; NMR spectrum: 1.50-1.78 (m, 2H), 1.81-1.94 (m, 2H), 2.74 (bs, 1H), 2.81-2.91 (m, 1H), 3.11 (bs, 1H), 3.77 (s, 3H), 3.82 (bs, 1H), 4.07-4.22 (m, 2H), 4.33-4.41 (m, 1H), 4.55 (bs, 1H), 6.89 (d, 1H), 7.03 (s, 1H), 7.16 (dd, 1H), 7.36 (bs, 1H), 7.44 (d, 1H), 8.27 (bs, 1H), 8.70 (s, 1H), 8.85 (d, 1H), 8.92 (s, 1H), 9.33 (bs, 1H); Mass spectrum: 484 (MH+).

### Example 241

### 1-[(1S,4S)-2-[4-[(5-chloro-4-pyrazolo[1,5-a]pyridin-3-yl-pyrimidin-2-yl)amino]-3-methoxy-phenyl]-2,5-diazabicyclo[2.2.1]heptan-5-yl]ethanone

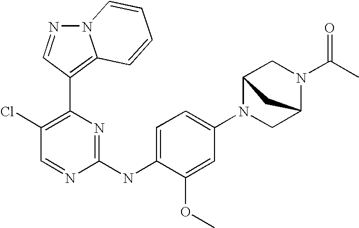

A mixture of 3-(2,5-dichloropyrimidin-4-yl)pyrazolo[1,5-a]pyridine (Method 35, 125 mg, 0.47 mmol), (1S,4S)-tert-butyl 5-(4-amino-3-methoxyphenyl)-2,5-diazabicyclo[2.2.1]heptane-2-carboxylate (obtained from (1S,4S)-tert-butyl 2,5-diazabicyclo[2.2.1]heptane-2-carboxylate following the procedure described in Method 7, 151 mg, 0.47 mmol) and pTsOH (179 mg, 0.94 mmol) in 2-pentanol (4 mL) was sealed into a microwave tube and heated at 140° C. for 3 hours in a microwave reactor. The reaction mixture was dissolved in water and a solution of sodium carbonate (10 ml) was added. The mixture was extracted with methylene chloride and the organic extracts were dried over magnesium sulfate and concentrated. The crude product was purified by flash chromatography on silica gel eluting with 0 to 3% methanolic ammonia (7 N) in methylene chloride to afford N-(4-((1S,4S)-2,5-diazabicyclo[2.2.1]heptan-2-yl)-2-methoxyphenyl)-5-chloro-4-(pyrazolo[1,5-a]pyridin-3-yl)pyrimidin-2-amine (70 mg, 33%). Acetyl chloride (0.012 mL, 0.16 mmol) was added to N-(4-((1S,4S)-2,5-diazabicyclo[2.2.1]heptan-2-yl)-2-methoxyphenyl)-5-chloro-4-(pyrazolo[1,5-a]pyridin-3-yl)pyrimidin-2-amine (70 mg, 0.16 mmol) and triethylamine (0.044 mL, 0.31 mmol) in methylene chloride (3 mL) at 0° C. The mixture was stirred at 0° C. for 1 hour then evaporated under reduce pressure. Methanol (2 ml) and a 2N aqueous solution of sodium hydroxide (0.3 ml) were added and the mixture was stirred at 25° C. for 30 minutes. The reaction mixture was dissolved in water, extracted with methylene chloride (25 mL) and the organic phase was dried over magnesium sulfate and concentrated. The crude product was purified by flash chromatography on silica gel eluting with 0 to 3% methanol in ethyl acetate to afford 1-((1S,4S)-5-(4-(5-chloro-4-(pyrazolo[1,5-a]pyridin-3-yl)pyrimidin-2-ylamino)-3-methoxyphenyl)-2,5-diazabicyclo[2.2.1]heptan-2-yl)ethanone (25 mg, 32%) as a pale yellow solid; NMR spectrum: (CDCl₃) 1.93 (s, 3H), 1.98-2.18 (m, 2H), 3.15-3.29 (m, 1H), 3.49-3.57 (m, 1H), 3.57-3.68 (m, 2H), 3.68-3.78 (m, 1H), 3.87 (s, 3H), 4.48 (bs, 1H), 6.17 (bs, 2H), 6.94 (dd, 1H), 7.20 (bs, 1H), 7.99 (d, 1H), 8.32 (s, 1H), 8.50 (d, 1H), 8.54 (dd, 1H), 8.92 (s, 1H); Mass spectrum: 490 (MH+).

### Example 242

### 2-[7-[4-[(5-chloro-4-pyrazolo[1,5-a]pyridin-3-yl-pyrimidin-2-yl)amino]-3-methoxy-phenyl]-9-oxa-3,7-diazabicyclo[3.3.1]nonan-3-yl]propane-1,3-diol

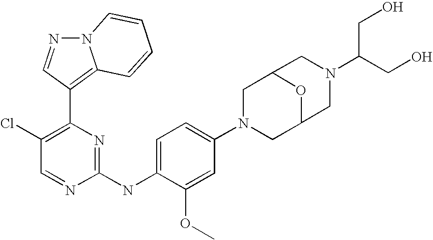

A mixture of 3-(2,5-dichloropyrimidin-4-yl)pyrazolo[1,5-a]pyridine (Method 35, 130 mg, 0.46 mmol), 2-(7-(4-amino-3-methoxyphenyl)-9-oxa-3,7-diazabicyclo[3.3.1]nonan-3-yl)propane-1,3-diol (164 mg, 0.46 mmol) and 4-methylbenzenesulfonic acid hydrate (104 mg, 0.55 mmol) in 2-pentanol (2 ml) was stirred at 130° C. for 4 h then at 140° C. for 5 hours. The reaction mixture was partitioned between saturated aqueous sodium bicarbonate and ethyl acetate and the organic layer was washed with brine, dried over MgSO4, filtrated and concentrated. The crude product was purified by flash chromatography on silica gel eluting with 0 to 8% MeOH/CH2Cl2 to afford 2-(7-(4-(5-chloro-4-(pyrazolo[1,5-a]pyridin-3-yl)pyrimidin-2-ylamino)-3-methoxyphenyl)-9-oxa-3,7-diazabicyclo[3.3.1]nonan-3-yl)propane-1,3-diol (159 mg, 63%) as a pale yellow solid; NMR spectrum: 2.55-2.64 (m, 1H), 2.83-2.95 (m, 2H), 3.02-3.11 (m, 2H), 3.11-3.20 (m, 2H), 3.33-3.43 (m partially hidden u) by H2O, 4H), 3.64-3.70 (m, 2H), 3.74 (s, 3H), 4.03 (bs, 2H), 4.25 (m, 2H), 6.47 (d, 1H), 6.56 (s, 1H), 7.10 (dd, 1H), 7.28 (d, 1H), 7.36 (dd, 1H), 8.29 (bs, 1H), 8.34 (s, 1H), 8.53 (s, 1H), 8.82 (d, 1H), 8.95 (s, 1H); Mass spectrum: 552 (MH+).

The 2-(7-(4-amino-3-methoxyphenyl)-9-oxa-3,7-diazabicyclo[3.3.1]nonan-3-yl)propane-1,3-diol used as a starting material was prepared from 2-(9-oxa-3,7-diazabicyclo[3.3.1]nonan-3-yl)propane-1,3-diol following the procedure described in Method 7.

2-(9-oxa-3,7-diazabicyclo[3.3.1]nonan-3-yl)propane-1,3-diol was prepared as follows:

A mixture of diethyl 2-bromomalonate (6.15 ml, 36.0 mmol), 3-benzyl-9-oxa-3,7-diazabicyclo[3.3.1]nonane dihydrochloride (WO-2001/028992, 10 g, 34.3 mmol) and potassium carbonate (23.7 g, 171 mmol) in acetonitrile (300 ml) was heated at 70° C. for 2 days. After filtration of the insoluble material, the solution was evaporated and the crude product was purified by flash chromatography on silica gel eluting with 0 to 100% EtOAc/petroleum ether to afford diethyl 2-(7-benzyl-9-oxa-3,7-diazabicyclo[3.3.1]nonan-3-yl)malonate (4.60 g, 35%) as a yellow oil.

Aluminum lithium hydride 1M in THF (14.3 ml, 14.3 mmol) was added dropwise to a stirred solution of diethyl 2-(7-benzyl-9-oxa-3,7-diazabicyclo[3.3.1]nonan-3-yl)malonate (4.5 g, 11.9 mmol) in THF (40 ml) at 0° C. under argon. The resulting solution was stirred at 35° C. for 3 hours. After cooling at 0° C., 10N NaOH was added until formation of a solid, and the mixture was filtered. The solution was extracted with EtOAc (twice) and the organic extracts were dried (MgSO₄), filtered and evaporated to dryness to give 2-(7-benzyl-9-oxa-3,7-diazabicyclo[3.3.1]nonan-3-yl)propane-1,3-diol (3.08 g, 88%) as an pale yellow oil. Cyclohexene (26.4 ml, 260.8 mmol) was added dropwise to a stirred suspension of 2-(7-benzyl-9-oxa-3,7-diazabicyclo[3.3.1]nonan-3-yl)propane-1,3-diol (3.05 g, 10.4 mmol) and dihydroxypalladium (0.439 g, 3.13 mmol) in EtOH (40 ml) at 80° C. under argon. The resulting suspension was stirred at 80° C. for 2 hours. After cooling, the mixture was filtered over celite and the filtrate evaporated to dryness to afford 2-(9-oxa-3,7-diazabicyclo[3.3.1]nonan-3-yl)propane-1,3-diol (2.3 g) as a pale yellow oil.

This product was used without further purification.

### Example 243

### 1-[4-[4-[[5-chloro-4-(7-pyrrolidin-1-ylimidazo[1,2-a]pyridin-3-yl)pyrimidin-2-yl]amino]-3-methoxy-phenyl]piperazin-1-yl]ethanone

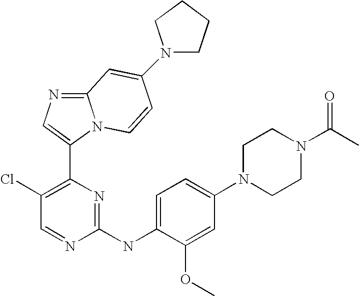

A mixture of 1-(4-(4-(5-chloro-4-(7-chloroimidazo[1,2-a]pyridin-3-yl)pyrimidin-2-ylamino)-3-methoxyphenyl)piperazin-1-yl)ethanone (Example 126.18, 100 mg, 0.20 mmol) and pyrrolidine (1.63 mL, 19.5 mmol) was sealed into a microwave tube and heated to 165° C. for 1 hour in the microwave reactor. The mixture was concentrated to dryness, the residue was taken up with methylene chloride and washed with a 2M aqueous solution of Na2CO3 (1 ml), dried over MgSO4, filtered and concentrated. The crude product was purified by flash chromatography on silica gel eluting with 0 to 4% methanol in methylene chloride to afford a solid which was dissolved in methylene chloride (1 ml) and ethanol (4 ml). This solution was concentrated until crystallisation occurred. The solid was filtered and dried under vacuum to afford 1-(4-(4-(5-chloro-4-(7-(pyrrolidin-1-yl)imidazo[1,2-a]pyridin-3-yl)pyrimidin-2-ylamino)-3-methoxyphenyl)piperazin-1-yl)ethanone (60 mg, 56%) as a yellow solid; NMR spectrum: 1.95-2.04 (m, 4H), 2.08 (s, 3H), 3.16-3.31 (m, 2H), 3.22-3.27 (m, 2H), 3.30-3.34 (m partially hidden by H2O, 4H), 3.60-3.66 (m, 4H), 3.74 (s, 3H), 6.21 (bs, 1H), 6.44 (d, 1H), 6.60 (dd, 1H), 6.77 (d, 1H), 7.27 (d, 1H), 8.30 (s, 1H), 8.58 (s, 1H), 8.63 (s, 1H), 9.23 (bs, 1H); Mass spectrum: 547 (MH+).

### Example 244 to 245

The following compounds were prepared using the procedure as described in Example 28.5 using the appropriate chloropyrimidines as set out in the following table instead of 3-(2,5-dichloropyrimidin-4-yl)imidazo[1,2-a]pyridine. The chloropyrimidines were prepared as described in Method 34.

| | | | | Chloropyrimidine |
|---|---|---|---|---|
| Ex. No. | Name | MH+ | NMR spectrum | starting material |
| 244 | 1-[4-[4-[[5-chloro-4-(6- | 511 | 1.45-1.60 (m, 1H), | 3-(2,5- |
| | fluoroimidazo[1,2- | | 1.60-1.73 (m, 1H), 1.78-1.88 (m, 2H), | dichloropyrimidin- |
| | a]pyridin-3- | | 2.66-2.75 (m, 1H), | 4-yl)-6-fluoro- |
| | yl)pyrimidin-2- | | 2.77-2.86 (m, 1H), 3.02-3.13 (m, 1H), | imidazo[1,2- |
| | yl]amino]-3-methoxy- | | 3.79 (s, 3H), 3.81 (bs, 1H), | a]pyridine |
| | phenyl]-1-piperidyl]-2- | | 4.07-4.21 (m, 2H), 4.49 (t, | |
| | hydroxy-ethanone | | 1H), 4.49-4.57 (m, 1H), | |
| | | | 6.84 (dd, 1H), 7.00 (s, 1H), | |
| | | | 7.44 (d, 1H), 7.60 (ddd, 1H), | |
| | | | 7.83 (dd, 1H), 8.51 (s, 1H), 8.78 (s, | |
| | | | 1H), 9.03 (s, 1H), 9.66 (bs, | |
| | | | 1H) | |
| 245 | 1-[4-[4-[[5-chloro-4-(7- | 507 | 1.53-1.67 (m, 1H), | 3-(2,5- |
| | methylimidazo[1,2- | | 1.65-1.75 (m, 1H), 1.79-1.89 (m, 2H), | dichloropyrimidin- |
| | a]pyridin-3- | | 2.42 (s, 3H), 2.68-2.77 (m, | 4-yl)-7-methyl- |
| | yl)pyrimidin-2- | | 1H), 2.80-2.89 (m, 1H), | imidazo[1,2- |
| | yl]amino]-3-methoxy- | | 3.04-3.15 (m, 1H), 3.80 (s, 3H), | a]pyridine |
| | phenyl]-1-piperidyl]-2- | | 3.83 (bs, 1H), 4.12 (dd, 1H), | |
| | hydroxy-ethanone | | 4.18 (dd, 1H), 4.50 (t, 1H), | |
| | | | 4.52-4.60 (m, 1H), 6.77 (d, | |
| | | | 1H), 6.86 (dd, 1H), 7.04 (s, | |
| | | | 1H), 7.49 (d, 1H), 7.56 (s, | |
| | | | 1H), 8.48 (s, 1H), 8.68 (s, | |
| | | | 1H), 8.87 (s, 1H), 9.49 (bs, | |
| | | | 1H) | |

### Example 246

The following compound was prepared using the procedure as described in Example 28.2 using the appropriate chloropyrimidines as set out in the following table instead of 3-(2,5-dichloropyrimidin-4-yl)imidazo[1,2-a]pyridine. The chloropyrimidines were prepared as described in Method 34.

| | | | | Chloropyrimidine |
|---|---|---|---|---|
| Ex. No. | Name | MH+ | NMR spectrum | starting material |
| 246 | (2R)-1-[4-[4-[[5-chloro- | 525 | 1.20 (d, 1.25H), 1.23 (d, 1.25H), | 3-(2,5- |
| | 4-(6-fluoroimidazo[1,2- | | 1.27-1.32 (m, 0.5H), | dichloropyrimidin- |
| | a]pyridin-3- | | 1.34 (d, 0.25H), 1.38 (d, 0.25H), | 4-yl)-6-fluoro- |
| | yl)pyrimidin-2- | | 1.44-1.72 (m, 2H), | imidazo[1,2- |
| | yl]amino]-3-methoxy | | 1.81-1.91 (m, 1.5H), 2.62-2.73 (m, 1H), | a]pyridine |
| | phenyl]-1-piperidyl]-2- | | 2.77-2.87 (m, 1H), | |
| | hydroxy-propan-1-one | | 3.07-3.15 (m, 1H), 3.79 (s, 3H), | |
| | | | 4.07-4.18 (m, 1H), 4.43-4.60 (m, | |
| | | | 2H), 4.84 (dd, 0.75H), | |
| | | | 5.45 (dd, 0.25H), 6.84 (d, 1H), | |
| | | | 6.99 (s, 1H), 7.44 (d, 1H), | |
| | | | 7.60 (ddd, 1H), 7.84 (dd, 1H), | |
| | | | 8.51 (s, 1H), 8.78 (s, 1H), | |
| | | | 9.04 (s, 1H), 9.66 (bs, 1H) | |

### Example 247

The following compound was prepared using the procedure as described in Example 4 using the appropriate chloropyrimidines as set out in the following table instead of 3-(2,5-dichloropyrimidin-4-yl)imidazo[1,2-a]pyridine. The chloropyrimidines were prepared as described in Method 34.

| | | | | Chloropyrimidine |
|---|---|---|---|---|
| Ex. No. | Name | MH+ | NMR spectrum | starting material |
| 247 | 5-chloro-4-(6- | 453 | 1.52 (dd, 1H), 1.57 (dd, 1H), | 3-(2,5- |
| | fluoroimidazo[1,2- | | 1.70-1.79 (m, 2H), | dichloropyrimidin- |
| | a]pyridin-3-yl)-N-[2- | | 2.55-2.68 (m, 3H) 2.99-3.09 (m, 2H), | 4-yl)-6-fluoro- |
| | methoxy-4-(4- | | 3.79 (s, 3H), 6.82 (dd, 1H), | imidazo[1,2- |
| | piperidyl)phenyl]pyrimidin- | | 6.96 (s, 1H), 7.46 (d, 1H), | a]pyridine |
| | 2-amine | | 7.56 (ddd, 1H), 7.80 (dd, 1H), | |
| | | | 8.49 (s, 1H), 8.76 (s, 1H), | |
| | | | 8.82 (s, 1H), 9.63 (s, 0.5H), | |
| | | | 9.65 (s, 0.5H) | |

### Example 248

The following compound was prepared using the procedure as described in Example 24.10 using the appropriate chloropyrimidines as set out in the following table instead of 3-(2,5-dichloropyrimidin-4-yl)imidazo[1,2-a]pyridine. The chloropyrimidines were prepared as described in Method 34.

| | | | | Chloropyrimidine |
|---|---|---|---|---|
| Ex. No. | Name | MH+ | NMR spectrum | starting material |
| 248 | 1-[4-[4-[[5-chloro-4- | 543 | 2.30 (s, 3H), 3.11-3.22 (m, | 3-(2,5- |
| | (6,8- | | 4H), 3.38 (s, 3H), | dichloropyrimidin- |
| | difluoroimidazo[1,2- | | 3.54-3.61 (m, 2H), 3.61-3.67 (m, 2H), | 4-yl)-6,8-difluoro- |
| | a]pyridin-3- | | 3.76 (s, 3H), 6.54 (dd, 1H), | imidazo[1,2- |
| | yl)pyrimidin-2- | | 6.71 (d, 1H), 7.26 (d, 1H), | a]pyridine |
| | yl]amino]-3-methoxy- | | 7.79 (ddd, 1H), 8.50 (s, 1H), | |
| | phenyl]piperazin-1-yl]- | | 8.77 (s, 1H), 9.00 (s, 1H), | |
| | 2- | | 8.49 (bs, 1H) | |
| | (methylamino)ethanone | | | |

### Example 249 to 251

The following compounds were prepared using the procedure as described in Example 40 using the appropriate chloropyrimidines as set out in the following table instead of 3-(2,5-dichloropyrimidin-4-yl)imidazo[1,2-a]pyridine. The chloropyrimidines were prepared as described in Method 34.

| | | | | Chloropyrimidine |
|---|---|---|---|---|
| Ex. No. | Name | MH+ | NMR spectrum | starting material |
| 249 | 1-[4-[4-[[5-chloro-4-(6- | 512 | 3.13-3.22 (m, 4H), | 3-(2,5- |
| | fluoroimidazo[1,2- | | 3.493.56 (m, 2H), 3.60-3.68 m, 2H), | dichloropyrimidin- |
| | a]pyridin-3- | | 3.76 (s, 3H), 4.15 (d, 2H), | 4-yl)-6-fluoro- |
| | yl)pyrimidin-2- | | 4.65 (t, 1H), 6.54 (dd, 1H), | imidazo[1,2- |
| | yl]amino]-3-methoxy- | | 6.71 (d, 1H), 7.27 (d, 1H), | a]pyridine |
| | phenyl]piperazin-1-yl]- | | 7.59 (ddd, 1H), 7.83 (dd, 1H), | |
| | 2-hydroxy-ethanone | | 8.47 (s, 1H), 8.78 (s, 1H), | |
| | | | 8.94 (s, 1H), 9.61 (bs, 1H) | |
| 250 | 1-[4-[4-[[5-chloro-4- | 530 | 3.11-3.23 (m, 4H), | 3-(2,5- |
| | (6,8- | | 3.46-3.58 (m, 2H), 3.58-3.68 (m, 2H), | dichloropyrimidin- |
| | difluoroimidazo[1,2- | | 3.76 (s, 3H), 4.15 (d, 2H), | 4-yl)-6,8-difluoro- |
| | a]pyridin-3- | | 4.65 (t, 1H), 6.54 (d, 1H), | imidazo[1,2- |
| | yl)pyrimidin-2- | | 6.71 (d, 1H), 7.25 (d, 1H), | a]pyridine |
| | yl]amino]-3-methoxy- | | 7.79 (dd, 1H), 8.50 (s, 1H), | |
| | phenyl]piperazin-1-yl]- | | 8.77 (s, 1H), 9.00 (s, 1H), | |
| | 2-hydroxy-ethanone | | 9.47 (bs, 1H) | |
| 251 | 1-[4-[4-[[5-chloro-4-(7- | 508 | 2.40 (s, 3H), 3.15-3.27 (m, | 3-(2,5- |
| | methylimidazo[1,2- | | 4H), 3.48-3.60 (m, 2H), | dichloropyrimidin- |
| | a]pyridin-3- | | 3.60-3.70 (m, 2H), 3.76 (s, 3H), | 4-yl)-7-methyl- |
| | yl)pyrimidin-2- | | 4.16 (d, 2H), 4.66 (t, 1H), | imidazo[1,2- |
| | yl]amino]-3-methoxy- | | 6.56 (dd, 1H), 6.72 (bs, 1H), | a]pyridine |
| | phenyl]piperazin-1-yl]- | | 6.74 (d, 1H), 7.30 (d, 1H), | |
| | 2-hydroxy-ethanone | | 7.53 (s, 1H), 8.42 (s, 1H), | |
| | | | 8.66 (s, 1H), 8.77 (s, 1H), | |
| | | | 9.43 (bs, 1H) | |

### Example 252 to 254

The following compounds were prepared using the procedure as described in Example using the appropriate chloropyrimidines as set out in the following table instead of 3-(2,5-dichloropyrimidin-4-yl)imidazo[1,2-a]pyridine. The chloropyrimidines were prepared as described in Method 34.

| | | | | Chloropyrimidine |
|---|---|---|---|---|
| Ex. No. | Name | MH+ | NMR spectrum | starting material |
| 252 | (2R)-2-amino-1-[4-[4- | 525 | 1.11 (d, 3H), 2.06 (bs, 2H), | 3-(2,5- |
| | [[5-chloro-4-(6- | | 3.09-3.22 (m, 4H), | dichloropyrimidin- |
| | fluoroimidazo[1,2- | | 3.56-3.73 (m, 4H), 3.76 (s, 3H), 3.83 (q, | 4-yl)-6-fluoro- |
| | a]pyridin-3- | | 1H), 6.55 (dd, 1H), 6.71 (d, | imidazo[1,2- |
| | yl)pyrimidin-2- | | 1H), 7.27 (d, 1H), 7.58 (ddd, | a]pyridine |
| | yl]amino]-3-methoxy- | | 1H), 7.83 (dd, 1H), 8.47 (s | |
| | phenyl]piperazin-1- | | 1H), 8.78 (s, 1H), 8.94 (s, | |
| | yl]propan-1-one | | 1H), 9.61 (bs, 1H) | |
| 253 | (2R)-2-amino-1-[4-[4- | 543 | 1.04 (d, 3H), 1.68 (bs, 2H), | 3-(2,5- |
| | [[5-chloro-4-(6,8- | | 3.02-3.16 (m, 4H), | dichloropyrimidin- |
| | difluoroimidazo[1,2- | | 3.49-3.66 (m, 4H), 3.69 (s, 3H), 3.74 (q, | 4-yl)-6,8-difluoro- |
| | a]pyridin-3- | | 1H), 6.48 (dd, 1H), 6.65 (d, | imidazo[1,2- |
| | yl)pyrimidin-2- | | 1H), 7.19 (d, 1H), 7.72 (ddd, | a]pyridine |
| | yl]amino]-3-methoxy- | | 1H), 8.44 (s, 1H), 8.71 (s, | |
| | phenyl]piperazin-1- | | 1H), 8.93 (s, 1H), 9.41 (bs, | |
| | yl]propan-1-one | | 1H) | |
| 254 | (2R)-2-amino-1-[4-[4- | 521 | 1.12 (d, 3H), 2.17 (bs, 2H), | 3-(2,5- |
| | [[5-chloro-4-(7- | | 2.40 (s, 3H), 3.11-3.28 (m, | dichloropyrimidin- |
| | methylimidazo[1,2- | | 4H), 3.59-3.78 (m, 4H), | 4-yl)-7-methyl- |
| | a]pyridin-3- | | 3.76 (s, 3H), 3.82 (q, 1H), | imidazo[1,2- |
| | yl)pyrimidin-2- | | 6.56 (dd, 1H), 6.71 (bs, 1H), | a]pyridine |
| | yl]amino]-3-methoxy- | | 6.75 (d, 1H), 7.29 (d, 1H), 7.53 (s, | |
| | phenyl]piperazin-1- | | 1H), 8.42 (s, 1H), 8.66 (s, | |
| | yl]propan-1-one | | 1H), 8.77 (s, 1H), 9.42 (bs, | |
| | | | 1H) | |

### Example 255 to 257

The following compounds were prepared using the procedure as described in Example 40 using the appropriate chloropyrimidines as set out in the following table instead of 3-(2,5-dichloropyrimidin-4-yl)imidazo[1,2-a]pyridine. The chloropyrimidines were prepared as described in Method 34.

| | | | | Chloropyrimidine |
|---|---|---|---|---|
| Ex. No. | Name | MH+ | NMR spectrum | starting material |
| 255 | (2R)-1-[4-[4-[[5-chloro- | 526 | 1.22 (d, 3H), 3.08-3.25 (m, | 3-(2,5- |
| | 4-(6-fluoroimidazo[1,2- | | 4H), 3.55-3.63 (m, 1H), | dichloropyrimidin- |
| | a]pyridin-3- | | 3.64-3.74 (m, 3H), 3.76 (s, 3H), | 4-yl)-6-fluoro- |
| | yl)pyrimidin-2- | | 4.46-4.53 (m, 1H), 4.99 (d, | imidazo[1,2- |
| | yl]amino]-3-methoxy- | | 1H), 6.55 (dd, 1H), 6.72 (d, | a]pyridine |
| | phenyl]piperazin-1-yl]- | | 1H), 7.27 (d, 1H), 7.58 (ddd, | |
| | 2-hydroxy-propan-1-one | | 1H), 7.82 (dd, 1H), 8.47 (s, | |
| | | | 1H), 8.78 (s, 1H), 8.94 (s, | |
| | | | 1H), 9.61 (bs, 1H) | |
| 256 | (2R)-1-[4-[4-[[5-chloro- | 544 | 1.22 (d, 3H), 3.07-3.25 (m, | 3-(2,5- |
| | 4-(6,8- | | 4H), 3.54-3.62 (m, 1H), | dichloropyrimidin- |
| | difluoroimidazo[1,2- | | 3.63-3.78 (m, 3H), 3.76 (s, 3H), | 4-yl)-6,8-difluoro- |
| | a]pyridin-3- | | 4.45-4.53 (m, 1H), 4.99 (d, | imidazo[1,2- |
| | yl)pyrimidin-2- | | 1H), 6.55 (dd, 1H), 6.71 (d, | a]pyridine |
| | yl]amino]-3-methoxy- | | 1H), 7.26 (d, 1H), 7.79 (dd, | |
| | phenyl]piperazin-1-yl]- | | 1H), 8.51 (s, 1H), 8.77 (s, | |
| | 2-hydroxy-propan-1-one | | 1H), 9.00 (s, 1H), 9.49 (bs, | |
| | | | 1H) | |
| 257 | (2R)-1-[4-[4-[[5-chloro- | 522 | 1.23 (d, 3H), 2.40 (s, 3H), | 3-(2,5- |
| | 4-(7-methylimidazo[1,2- | | 3.13-3.28 (m, 4H), | dichloropyrimidin- |
| | a]pyridin-3- | | 3.56-3.66 (m, 1H), 3.65-3.79 (m, 3H), | 4-yl)-7-methyl- |
| | yl)pyrimidin-2- | | 3.76 (s, 3H), 4.50 (q, 1H), | imidazo[1,2- |
| | yl]amino]-3-methoxy- | | 5.01 (bs, 1H), 6.57 (dd, 1H), | a]pyridine |
| | phenyl]piperazin-1-yl]- | | 6.71 (bs 1H), 6.75 (d, 1H), | |
| | 2-hydroxy-propan-1-one | | 7.29 (d, 1H), 7.53 (s, 1H), | |
| | | | 8.42 (s, 1H), 8.66 (s, 1H), | |
| | | | 8.77 (s, 1H), 9.42 (bs, 1H) | |

### Example 258

### (2S)-2-amino-1-[4-[4-[[5-chloro-4-(7-methylimidazo[1,2-a]pyridin-3-yl)pyrimidin-2-yl]amino]-3-methoxy-phenyl]piperazin-1-yl]propan-1-one

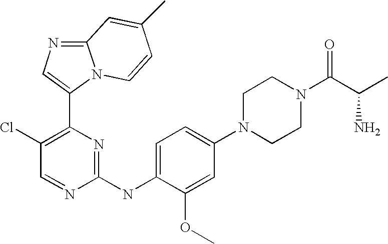

This compound was prepared using the procedure as described in Example 24.5 using 3-(2,5-dichloropyrimidin-4-yl)-7-methyl-imidazo[1,2-a]pyridine (Method 34) instead of 3-(2,5-dichloropyrimidin-4-yl)imidazo[1,2-a]pyridine; NMR spectrum: 1.11 (d, 3H), 1.80 (m, 2H), 2.40 (s, 3H), 3.13-3.27 (m, 4H), 3.59-3.74 (m, 4H), 3.77 (s, 3H), 3.81 (q, 1H), 6.56 (dd, 1H), 6.71 (bs, 1H), 6.75 (d, 1H), 7.29 (d, 1H), 7.53 (s, 1H), 8.42 (s, 1H), 8.67 (s, 1H), 8.78 (s, 1H), 9.42 (bs, 1H); Mass spectrum: 521 (MH+).

### Example 259

### 2-[4-[4-[[5-chloro-4-(6-fluoroimidazo[1,2-a]pyridin-3-yl)pyrimidin-2-yl]amino]-3-methoxy-phenyl]piperazin-1-yl]-2-methyl-propan-1-ol

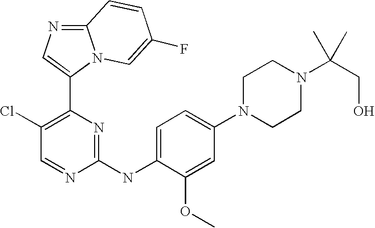

This compound was prepared using the procedure as described in Example 228 using 3-(2,5-dichloropyrimidin-4-yl)-6-fluoro-imidazo[1,2-a]pyridine (Method 34) instead of 3-(2-chloro-5-fluoro-pyrimidin-4-yl)imidazo[1,2-a]pyridine; NMR spectrum: 0.99 (s, 6H), 2.65-2.77 (m, 4H), 3.09-3.18 (m, 4H), 3.31 (d partially hidden by H2O, 2H), 3.74 (s, 3H), 4.31 (t, 1H), 6.49 (dd, 1H), 6.64 (d, 1H), 7.22 (d, 1H), 7.59 (ddd, 1H), 7.82 (dd, 1H), 8.46 (s, 1H), 8.77 (s, 1H), 8.90 (bs, 1H), 9.58 (bs, 1H); Mass spectrum: 526 (MH+).

### Example 260

### 2-[4-[4-[[4-(6-fluoroimidazo[1,2-a]pyridin-3-yl)-5-methyl-pyrimidin-2-yl]amino]-3-methoxy-phenyl]piperazin-1-yl]-2-methyl-propan-1-ol

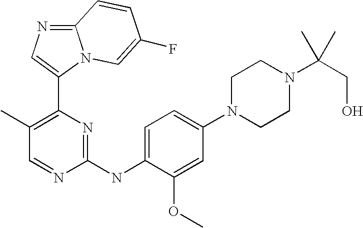

This compound was prepared using the procedure as described in Example 228 using 3-(2-chloro-5-methyl-pyrimidin-4-yl)-6-fluoro-imidazo[1,2-a]pyridine instead of 3-(2-chloro-5-fluoro-pyrimidin-4-yl)imidazo[1,2-a]pyridine. 3-(2-chloro-5-methyl-pyrimidin-4-yl)-6-fluoro-imidazo[1,2-a]pyridine was prepared following the same procedure as for 3-(2,5-dichloropyrimidin-4-yl)-6-fluoro-imidazo[1,2-a]pyridine in Method 34 using 2,4-dichloro-5-methyl-pyrimidine instead of 2,4,5-trichloropyrimidine; NMR spectrum: 0.99 (s, 6H), 2.36 (s, 3H), 2.68-2.74 (m, 4H), 3.08-3.15 (m, 4H), 3.32 (d partially hidden by H2O, 2H), 3.76 (s, 3H), 4.30 (t, 1H), 6.48 (dd, 1H), 6.64 (d, 1H), 7.32 (d, 1H), 7.53 (ddd, 1H), 7.78 (dd, 1H), 8.30 (s, 1H), 8.31 (s, 1H), 8.35 (s, 1H), 9.67 (bs, 1H); Mass spectrum: 506 (MH+).

### Example 261

### 2-[4-[4-[[5-fluoro-4-(6-fluoroimidazo[1,2-a]pyridin-3-yl)pyrimidin-2-yl]amino]-3-methoxy-phenyl]piperazin-1-yl]-2-methyl-propan-1-ol

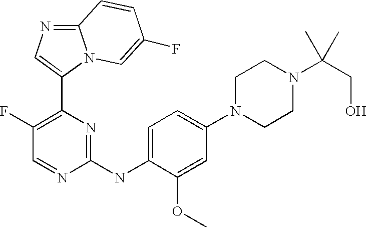

This compound was prepared using the procedure as described in Example 228 using 3-(2-chloro-5-fluoro-pyrimidin-4-yl)-6-fluoro-imidazo[1,2-a]pyridine instead of 3-(2-chloro-5-fluoro-pyrimidin-4-yl)imidazo[1,2-a]pyridine. 3-(2-chloro-5-fluoro-pyrimidin-4-yl)-6-fluoro-imidazo[1,2-a]pyridine was prepared following the same procedure as for 3-(2,5-dichloropyrimidin-4-yl)-6-fluoro-imidazo[1,2-a]pyridine in Method 34 using 2,4-dichloro-5-fluoro-pyrimidine instead of 2,4,5-trichloropyrimidine; NMR spectrum: 0.92 (s, 6H), 2.62-2.68 (m, 4H), 3.03-3.10 (m, 4H), 3.24 (d partially hidden by H2O, 2H), 3.67 (s, 3H), 4.24 (t, 1H), 6.44 (dd, 1H), 6.58 (d, 1H), 7.20 (d, 1H), 7.55 (ddd, 1H), 7.78 (dd, 1H), 8.32 (d, 1H), 8.39 (d, 1H), 8.67 (s, 1H), 9.72 (bs, 1H); Mass spectrum: 510 (MH+).

### Example 262

### 2-[4-[3-methoxy-4-[[5-methyl-4-(7-methylimidazo[1,2-a]pyridin-3-yl)pyrimidin-2-yl]amino]phenyl]piperazin-1-yl]-2-methyl-propan-1-ol

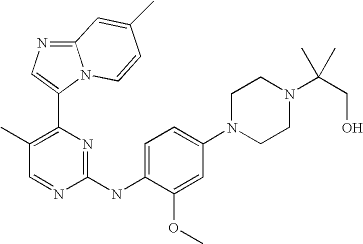

This compound was prepared using the procedure as described in Example 228 using 3-(2-chloro-5-methyl-pyrimidin-4-yl)-7-methyl-imidazo[1,2-a]pyridine instead of 3-(2-chloro-5-fluoro-pyrimidin-4-yl)imidazo[1,2-a]pyridine.

3-(2-chloro-5-methyl-pyrimidin-4-yl)-7-methyl-imidazo[1,2-a]pyridine was prepared following the same procedure as for 3-(2,5-dichloro-pyrimidin-4-yl)-7-methyl-imidazo[1,2-a]pyridine in Method 34 using 2,4-dichloro-5-methyl-pyrimidine instead of 2,4,5-trichloropyrimidine; NMR spectrum: 1.00 (s, 6H), 2.35 (s, 3H), 3.41 (s, 3H), 2.69-278 (m, 4H), 3.10-3.20 (m, 4H), 3.33 (bs, 2H), 3.77 (s, 3H), 4.31 (t, 1H), 6.50 (dd, 1H), 6.67 (d, 1H), 6.72 (d, 1H), 7.40 (d, 1H), 7.50 (s, 1H), 8.18 (s, 1H), 8.19 (s, 1H), 8.27 (s, 1H), 9.48 (bs, 0.5H), 9.49 (bs, 0.5H); Mass spectrum: 502 (MH+).

### Example 263

### 2-[4-[4-[[5-chloro-4-(6-fluoroimidazo[1,2-a]pyridin-3-yl)pyrimidin-2-yl]amino]-3-methoxy-phenyl]piperazin-1-yl]ethanol

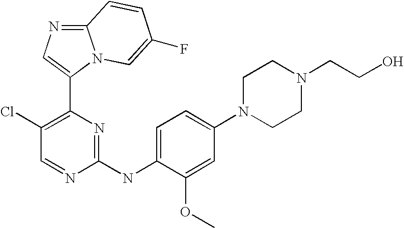

This compound was prepared using the procedure as described in Example 3.45 using 3-(2,5-dichloropyrimidin-4-yl)-6-fluoro-imidazo[1,2-a]pyridine (Method 34) instead of 3-(2,5-dichloropyrimidin-4-yl)imidazo[1,2-a]pyridine; NMR spectrum: 2.45 (t, 2H), 2.55-2.61 (m, 4H), 3.12-3.19 (m, 4H), 3.52-3.58 (m, 2H), 3.75 (s, 3H), 4.44 (t, 1H), 6.51 (dd, 1H), 6.66 (d, 1H), 7.22 (d, 1H), 7.59 (ddd, 1H), 7.82 (dd, 1H), 8.46 (s, 1H), 8.78 (s, 1H), 8.91 (s, 1H), 9.58 (bs, 1H); Mass spectrum: 498 (MH+).

### Example 264

### 2-[4-[4-[[4-(6-fluoroimidazo[1,2-a]pyridin-3-yl)-5-methyl-pyrimidin-2-yl]amino]-3-methoxy-phenyl]piperazin-1-yl]ethanol

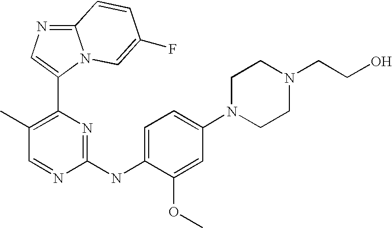

This compound was prepared using the procedure as described in Example 3.45 using 3-(2-chloro-5-methyl-pyrimidin-4-yl)-6-fluoro-imidazo[1,2-a]pyridine instead of 3-(2,5-dichloropyrimidin-4-yl)imidazo[1,2-a]pyridine. 3-(2-chloro-5-methyl-pyrimidin-4-yl)-6-fluoro-imidazo[1,2-a]pyridine was prepared following the same procedure as for 3-(2,5-dichloropyrimidin-4-yl)-6-fluoro-imidazo[1,2-a]pyridine in Method 34 using 2,4-dichloro-5-methyl-pyrimidine instead of 2,4,5-trichloropyrimidine; NMR spectrum: 2.36 (s, 3H), 2.45 (t, 2H), 2.55-2.60 (m, 4H), 3.10-3.17 (m, 4H), 3.52-3.59 (m, 2H), 3.76 (s, 3H), 4.45 (bs, 1H), 6.49 (dd, 1H), 6.65 d, 1H), 7.33 (d, 1H), 7.53 (ddd, 1H), 7.78 (ddd, 1H), 8.32 (s, 1H), 8.33 (s, 1H), 8.35 (s, 1H), 9.67 (bs, 1H); Mass spectrum: 478 (MH+).

### Example 265

### 5-chloro-4-(6,8-difluoroimidazo[1,2-a]pyridin-3-yl)-N-(2-methoxy-4-piperazin-1-yl-phenyl)pyrimidin-2-amine

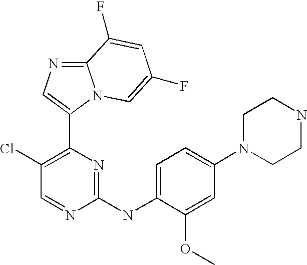

This compound was prepared using the procedure as described in Example 2 using 3-(2,5-dichloropyrimidin-4-yl)-6,8-difluoro-imidazo[1,2-a]pyridine (Method 34) instead of 3-(2,5-dichloropyrimidin-4-yl)imidazo[1,2-a]pyridine; NMR spectrum: 2.67 (bs, 1H), 2.83-2.89 (m, 4H), 3.04-3.10 (m, 4H), 3.74 (s, 3H), 6.50 (dd, 1H), 6.65 (d, 1H), 7.21 (d, 1H), 7.78 (ddd, 1H), 8.50 (s, 1H), 8.77 (s, 1H), 8.96 (s, 1H), 9.49 (bs, 1H); Mass spectrum: 472 (MH+).

### Example 266

### 3-[5-chloro-2-(2-methoxy-4-piperazin-1-yl-anilino)pyrimidin-4-yl]imidazo[1,2-a]pyridine-6-carbonitrile

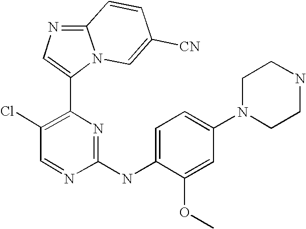

This compound was prepared using the procedure as described in Example 2 using 3-(2,5-dichloropyrimidin-4-yl)imidazo[1,2-a]pyridine-6-carbonitrile (Method 34) instead of 3-(2,5-dichloropyrimidin-4-yl)imidazo[1,2-a]pyridine; NMR spectrum: 2.67 (bs, 1H), 2.83-2.89 (m, 4H), 3.04-3.10 (m, 4H), 3.74 (s, 3H), 6.50 (dd, 1H), 6.65 (d, 1H), 7.21 (d, 1H), 7.78 (ddd, 1H), 8.50 (s, 1H), 8.77 (s, 1H), 8.96 (s, 1H), 9.49 (bs, 1H); Mass spectrum: 461 (MH+).

### Example 267

### (2R)-3-[4-[4-[(4-imidazo[1,2-a]pyridin-3-yl-5-methyl-pyrimidin-2-yl)amino]-3-methoxy-phenyl]piperazin-1-yl]propane-1,2-diol

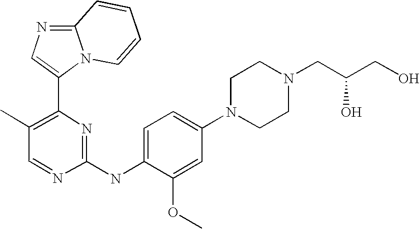

This compounds was prepared using the procedure as described in Example 53 using 3-(2-chloro-5-methyl-pyrimidin-4-yl)imidazo[1,2-a]pyridine (Method 5) instead of 3-(2,5-dichloropyrimidin-4-yl)imidazo[1,2-a]pyridine; NMR spectrum: 2.30 (dd, 1H), 2.36 (s, 3H), 2.45 (dd, 1H), 2.55-2.66 (m, 4H), 3.13-3.20 (m, 4H), 3.29-3.39 (m partially hidden by H2O, 2H), 3.63-3.71 (m, 1H), 3.77 (s, 3H), 4.44 (d, 1H), 4.55 (bs, 1H), 6.50 (dd, 1H), 6.68 (d, 1H), 6.88 (dd, 1H), 7.41 (d, 1H), 7.44 (ddd, 1H), 7.71 (d, 1H), 8.21 (s, 1H), 8.25 (s, 1H), 8.29 (s, 1H), 8.59 (s, 0.5H), 8.60 (s, 0.5H); Mass spectrum: 490 (MH+).

### Example 268

### (2S)-3-[4-[4-[(4-imidazo[1,2-a]pyridin-3-yl-5-methyl-pyrimidin-2-yl)amino]-3-methoxy-phenyl]piperazin-1-yl]propane-1,2-diol

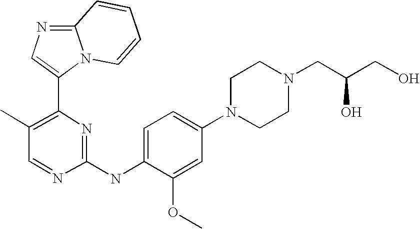

This compounds was prepared using the procedure used for Example 267 using (R)-oxiran-2-ylmethanol instead of (S)-oxiran-2-ylmethanol; NMR spectrum: 2.30 (dd, 1H), 2.36 (s, 3H), 2.45 (dd, 1H), 2.55-2.66 (m, 4H), 3.13-3.20 (m, 4H), 3.29-3.39 (m partially hidden by H2O, 2H), 3.63-3.71 (m, 1H), 3.77 (s, 3H), 4.44 (d, 1H), 4.55 (bs, 1H), 6.50 (dd, 1H), 6.68 (d, 1H), 6.88 (dd, 1H), 7.41 (d, 1H), 7.44 (ddd, 1H), 7.71 (d, 1H), 8.21 (s, 1H), 8.25 (s, 1H), 8.29 (s, 1H), 8.59 (s, 0.5H), 8.60 (s, 0.5H); Mass spectrum: 490 (MH+).

### Example 269

### 2-[4-[4-[(4-imidazo[1,2-a]pyridin-3-yl-5-methyl-pyrimidin-2-yl)amino]-3-methoxy-phenyl]piperazin-1-yl]propane-1,3-diol

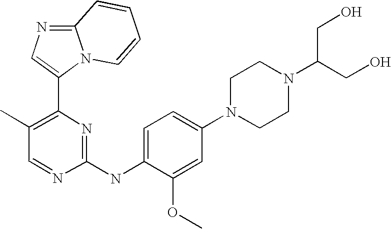

A mixture of potassium carbonate (3.14 g, 22.6 mmol), diethyl 2-bromomalonate (2.03 ml, 11.9 mmol) and 1-benzylpiperazine (1.97 ml, 11.3 mmol) in acetonitrile (100 ml) was stirred at 70° C. overnight. After cooling, the mixture was filtered, the filtrate was concentrated to dryness then purified by flash chromatography on silica gel eluting with 10 to 20% ethyl acetate in methylene chloride to afford diethyl 2-(4-benzylpiperazin-1-yl)malonate (3.20 g, 84%) as a colorless liquid.

A solution of diethyl 2-(4-benzylpiperazin-1-yl)malonate (1 g, 2.99 mmol) in THF (7 ml) was added dropwise to aluminum(III) lithium hydride 1M in THF (3.59 ml, 3.59 mmol) over a period of 10 minutes at 0° C. under argon. The resulting solution was stirred at 35° C. for 2 hours. After cooling to 0° C., NaOH 2N then NaOH 6N were added dropwise until a white solid salt was obtained. The salt was filtered, washed with diethyl ether and the filtrate was concentrated to dryness to afford 2-(4-benzylpiperazin-1-yl)propane-1,3-diol (0.760 g, 100%) as a colorless oil.

Palladium hydroxide on carbon 20% (126 mg, 0.90 mmol) and cyclohexene (7.59 ml, 74.9 mmol), were added to a stirred solution of 2-(4-benzylpiperazin-1-yl)propane-1,3-diol (750 mg, 3.00 mmol) in ethanol (14 ml). The resulting suspension was stirred at 80° C. for 3 hours. After cooling the catalyst was filtered on celite and the filtrate was concentrated to dryness to afford 2-(piperazin-1-yl)propane-1,3-diol (472 mg, 98%) as a yellow oil.

A mixture of 4-fluoro-2-methoxy-1-nitrobenzene (420 mg, 2.45 mmol), 2-(piperazin-1-yl)propane-1,3-diol (472 mg, 2.95 mmol) and N,N-diisopropylethylamine (0.513 mL, 2.95 mmol) in DMA (2 mL) was heated to 90° C. for 3 hours. After removal of the volatiles, the residue was purified by flash chromatography on silica gel eluting with 5 to 10% methanol in methylene chloride to afford 2-(4-(3-methoxy-4-nitrophenyl)piperazin-1-yl)propane-1,3-diol (423 mg, 55%) as a yellow solid.

A suspension of 2-(4-(3-methoxy-4-nitrophenyl)piperazin-1-yl)propane-1,3-diol (415 mg, 1.33 mmol) and platinum(IV) oxide (15.1 mg, 0.07 mmol) in ethanol (50 ml) was hydrogenated under 1.3 bars at 25° C. for 1H30. The suspension was filtered, the solvent was evaporated to dryness to afford 2-(4-(4-amino-3-methoxyphenyl)piperazin-1-yl)propane-1,3-diol (373 mg, 99%); NMR Spectrum: 2.50-2.52 (m, 1H), 2.72-2.77 (m, 4H), 2.88-2.93 (m, 4H), 3.44-3.56 (m, 4H), 3.73 (s, 3H), 4.19 (bs, 2H), 4.26 (t, 2H), 6.27 (dd, 1H), 6.47 (d, 1H), 6.51 (d, 1H).

A mixture of 3-(2-chloro-5-methylpyrimidin-4-yl)imidazo[1,2-a]pyridine (130 mg, 0.53 mmol), 2-(4-(4-amino-3-methoxyphenyl)piperazin-1-yl)propane-1,3-diol (149 mg, 0.53 mmol) and 4-methylbenzenesulfonic acid hydrate (152 mg, 0.80 mmol) in 2-pentanol (1.2 ml) was stirred at 140° C. for 3 hours. 2-pentanol was evaporated and the residue was purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent. The resulting product was further purified by flash chromatography on silica gel eluting with 5 to 10% methanol in methylene chloride to afford 2-(4-(4-(4-(imidazo[1,2-a]pyridin-3-yl)-5-methylpyrimidin-2-ylamino)-3-methoxyphenyl)piperazin-1-yl)propane-1,3-diol (150 mg, 57%); NMR spectrum: 2.36 (s, 3H), 2.51-2.59 (m, 1H), 2.76-2.85 (m, 4H), 3.09-3.18 (m, 4H), 3.46-3.60 (m, 4H), 3.77 (s, 3H), 4.32 (t, 2H), 6.49 (dd, 1H), 6.66 (d, 1H), 6.88 (dd, 1H), 7.40 (d, 1H), 7.44 (ddd, 1H), 7.71 (d, 1H), 8.20 (s, 1H), 8.25 (s, 1H), 8.29 (s, 1H), 9.59 (bs, 0.5H), 9.60 (bs, 0.5H); Mass spectrum: 490 (MH+).

### Example 270

### 2-[(2R)-4-[4-[(4-imidazo[1,2-a]pyridin-3-yl-5-methyl-pyrimidin-2-yl)amino]-3-methoxy-phenyl]-2-methyl-piperazin-1-yl]ethanol

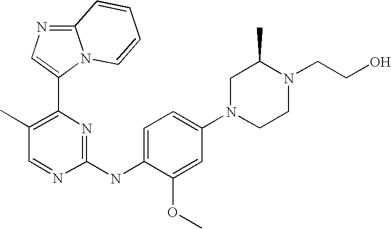

A mixture of (R)-tert-butyl 3-methylpiperazine-1-carboxylate (2 g, 9.99 mmol), potassium carbonate (1.79 g, 12.9 mmol), potassium iodide (1.65 g, 9.99 mmol) and methyl 2-bromoacetate (2.84 mL, 29.9 mmol) in acetone (10 mL) was heated at 40° C. for 20 hours. The reaction mixture was cooled to RT and concentrated to dryness. The resulting solid was triturated with methylene chloride and filtered. The filtrate was evaporated under reduced pressure to give (R)-tert-butyl 4-(2-methoxy-2-oxoethyl)-3-methylpiperazine-1-carboxylate (2.00 g, 73%).

A mixture of (R)-tert-butyl 4-(2-methoxy-2-oxoethyl)-3-methylpiperazine-1-carboxylate (1.71 g, 6.28 mmol) and aluminum(III) lithium hydride (0.61 mL, 12.5 mmol) in THF (30 mL) was heated at 60° C. for 2 hours. The reaction mixture was allowed to cool to room temperature and quenched with 10N NaOH under stirring. THF was evaporated, the pH was adjusted at pH˜8 and the aqueous layer was extracted with ethyl acetate. The combined organic phases were dried over magnesium sulfate and concentrated to afford the crude product (R)-tert-butyl 4-(2-hydroxyethyl)-3-methylpiperazine-1-carboxylate (1.30 g, 85%). (R)-tert-butyl 4-(2-hydroxyethyl)-3-methylpiperazine-1-carboxylate (1.3 g, 5.16 mmol) was dissolved in methylene chloride (20 mL) and TFA (5 mL) and stirred at 25° C. for 16 hours. The volatiles were evaporated and dried under vacuum to give crude (R)-2-(2-methylpiperazin-1-yl)ethanol which was dissolved in DMA (3 mL). 4-fluoro-2-methoxy-1-nitrobenzene (0.880 g, 5.15 mmol) and cesium carbonate (3.35 g, 10.3 mmol) were added and the mixture was stirred at 100° C. for 3 hours. The reaction mixture was diluted with water (pH˜8.5) and the aqueous layer was extracted with methylene chloride. The combined organic phases were dried over magnesium sulfate and concentrated to afford the crude product which was purified by flash chromatography on silica gel eluting with 0 to 10% methanol in ethyl acetate. The solvent was evaporated to dryness to afford (R)-2-(4-(3-methoxy-4-nitrophenyl)-2-methylpiperazin-1-yl)ethanol (0.60 g, 39%).

A suspension of (R)-2-(4-(3-methoxy-4-nitrophenyl)-2-methylpiperazin-1-yl)ethanol (708 mg, 2.04 mmol) and platinum(IV) oxide (100 mg) in EtOAc (5 mL) and ethanol (5 mL) was hydrogenated under 1.2 bar at 25° C. for 4 hours. The resulting suspension was filtered and the filtrate was concentrated to dryness to afford the crude (R)-2-(4-(4-amino-3-methoxyphenyl)-2-methylpiperazin-1-yl)ethanol (556 mg, 100%).

A mixture of 3-(2-chloro-5-methylpyrimidin-4-yl)imidazo[1,2-a]pyridine (150 mg, 0.61 mmol), (R)-2-(4-(4-amino-3-methoxyphenyl)-2-methylpiperazin-1-yl)ethanol (163 mg, 0.61 mmol) and pTsOH (128 mg, 0.67 mmol) in 2-pentanol (3 mL) was heated at 140° C. for 4 hours. The solvent was evaporated under reduced pressure and the residue was purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent. The fractions were evaporated to dryness to afford the desired product which was further purified by flash chromatography on silica gel eluting with 0 to 12% methanol in methylene chloride to afford 2-42R)-4-(4-(4-(imidazo[1,2-a]pyridin-3-yl)-5-methylpyrimidin-2-ylamino)-3-methoxyphenyl)-2-methylpiperazin-1-yl)ethanol (186 mg, 64%) as a pale yellow solid; NMR spectrum: 1.09 (d, 3H), 2.28-2.36 (m, 1H), 2.36 (s, 3H), 2.46 (ddd, 1H), 2.51-2.61 (m partially hidden by DMSOd5, 2H), 2.77-2.88 (m, 2H), 2.91-3.01 (m, 1H), 2.43-2.50 (m, 2H), 2.50-2.59 (m, 2H), 3.78 (s, 3H), 4.38-4.51 (m, 1H), 6.50 (dd, 1H), 6.68 (d, 1H), 6.88 (dd, 1H), 7.21 (d, 1H), 7.44 (dd, 1H), 7.72 (d, 1H), 8.21 (s, 1H), 8.26 (s, 1H), 8.30 (s, 1H), 9.59 (bs, 0.5H), 9.60 (bs, 0.5H); Mass spectrum: 474 (MH+).

### Example 271

### 2-[(2S)-4-[4-[(4-imidazo[1,2-a]pyridin-3-yl-5-methyl-pyrimidin-2-yl)amino]-3-methoxy-phenyl]-2-methyl-piperazin-1-yl]ethanol

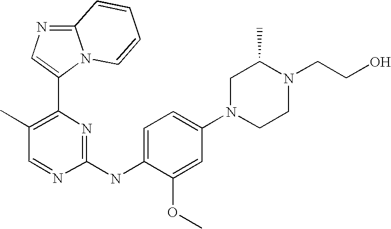

This compounds was prepared using the procedure used for Example 270 using (S)-tert-butyl 3-methylpiperazine-1-carboxylate instead of (R)-tert-butyl 3-methylpiperazine-1-carboxylate; NMR spectrum: 1.10 (s, 3H), 2.33 (bs, 1H), 2.37 (s, 3H), 2.42-2.52 (m partially hidden by DMSOd5, 2H), 2.73-2.89 (m, 2H), 2.92-3.01 (m, 1H), 3.35-3.43 (m partially hidden by H2O, 1H), 3.42-3.50 (m, 2H), 3.50-3.60 (m, 2H), 3.78 (s, 3H), 4.42 (bs, 1H), 6.50 (d, 1H), 6.68 (s, 1H), 6.88 (bs, 1H), 7.41 (d, 1H), 7.43 (dd, 1H), 7.72 (d, 1H), 8.21 (s, 1H), 8.26 (s, 1H), 8.30 (s, 1H), 9.60 (s, 1H); Mass spectrum: 474 (MH+).

### Example 272

### 2-[(2R)-4-[4-[(5-fluoro-4-imidazo[1,2-a]pyridin-3-yl-pyrimidin-2-yl)amino]-3-methoxy-phenyl]-2-methyl-piperazin-1-yl]ethanol

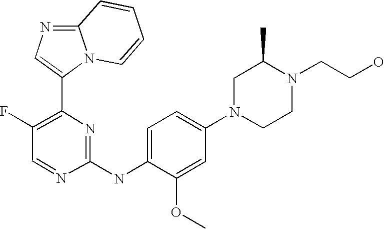

This compounds was prepared using the procedure used for Example 270 using 3-(2-chloro-5-fluoropyrimidin-4-yl)imidazo[1,2-a]pyridine instead of 3-(2-chloro-5-methylpyrimidin-4-yl)imidazo[1,2-a]pyridine; NMR spectrum: 1.11 (d, 3H), 2.31-2.39 (m, 1H), 2.45-2.50 (m, 1H), 2.51-2.60 (m, 2H), 2.78-2.90 (m, 2H), 2.95-3.02 (m, 1H), 3.45-3.59 (m, 4H), 3.76 (s, 3H), 4.43 (t, 1H), 6.54 (dd, 1H), 6.69 (d, 1H), 6.94 (dd, 1H), 7.33 (d, 1H), 7.54 (dd, 1H), 7.79 (d, 1H), 8.37 (d, 1H), 8.44 (d, 1H), 8.61 (s, 1H), 9.76 (bs, 1H); Mass spectrum: 478 (MH+).

### Example 273

### 2-[(2S)-4-[4-[(5-fluoro-4-imidazo[1,2-a]pyridin-3-yl-pyrimidin-2-yl)amino]-3-methoxy-phenyl]-2-methyl-piperazin-1-yl]ethanol

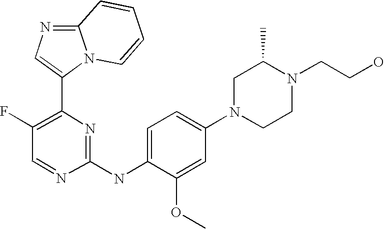

This compounds was prepared using the procedure used for Example 271 using 3-(2-chloro-5-fluoropyrimidin-4-yl)imidazo[1,2-a]pyridine instead of 3-(2-chloro-5-methylpyrimidin-4-yl)imidazo[1,2-a]pyridine; NMR spectrum: 1.11 (d, 3H), 2.31-2.39 (m, 1H), 2.45-2.50 (m, 1H), 2.51-2.60 (m, 2H), 2.78-2.90 (m, 2H), 2.95-3.02 (m, 1H), 3.45-3.59 (m, 4H), 3.76 (s, 3H), 4.43 (t, 1H), 6.54 (dd, 1H), 6.69 (d, 1H), 6.94 (dd, 1H), 7.33 (d, 1H), 7.54 (dd, 1H), 7.79 (d, 1H), 8.37 (d, 1H), 8.44 (d, 1H), 8.61 (s, 1H), 9.76 (bs, 1H); Mass spectrum: 478 (MH+).

### Example 274

### 2-[4-[4-[(4-imidazo[1,2-a]pyridin-3-yl-5-methyl-pyrimidin-2-yl)amino]-3-methoxy-phenyl]piperazin-1-yl]propan-1-ol

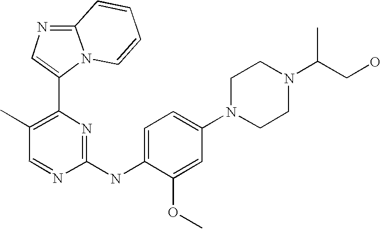

This compounds was prepared using the procedure used for Example 228 using 3-(2-chloro-5-methylpyrimidin-4-yl)imidazo[1,2-a]pyridine instead of 3-(2-chloro-5-fluoropyrimidin-4-yl)imidazo[1,2-a]pyridine and 2-(piperazin-1-yl)propan-1-ol instead of 2-methyl-2-(piperazin-1-yl)propan-1-ol; NMR spectrum: 0.98 (d, 3H), 2.36 (s, 3H), 3.58-3.73 (m, 5H), 3.10-3.19 (m, 4H), 3.27-3.34 (m partially hidden by H2O, 1H), 3.48-3.56 (m, 1H), 3.77 (s, 3H), 4.33 (dd, 1H), 6.50 (dd, 1H), 6.67 (d, 1H), 6.87 (dd, 1H), 7.40 (d, 1H), 7.44 (ddd, 1H), 7.71 (d, 1H), 8.21 (s, 1H), 8.25 (s, 1H), 8.29 (s, 1H), 9.59 (bs, 0.5H), 9.60 (bs, 0.5H); Mass spectrum: 474 (MH+).

The 2-(piperazin-1-yl)propan-1-ol used as a starting material was prepared as follows:

A mixture of 1-benzylpiperazine (2.46 mL, 14.1 mmol), ethyl 2-bromopropanoate (2.21 mL, 17.0 mmol) and N-ethyl-N-isopropylpropan-2-amine (4.94 mL, 28.3 mmol) in DMF (40 mL) was stirred at 110° C. for 4 hours. The reaction mixture was allowed to cool to room temperature, water was added and the mixture extracted with ethyl acetate. The combined organic extracts were washed with water and brine, dried over magnesium sulfate and concentrated to dryness to afford ethyl 2-(4-benzylpiperazin-1-yl)propanoate (4.0 g, 100%) as an orange liquid.

Aluminum(III) lithium hydride 1M in THF (8.47 ml, 8.47 mmol) was added dropwise to a stirred solution of ethyl 2-(4-benzylpiperazin-1-yl)propanoate (3.9 g, 14.1 mmol) in THF (25 ml) over a period of 10 minutes at 0° C. under argon. The resulting solution was stirred at 50° C. for 2 hours. After cooling to 0° C., NaOH 6N was added dropwise until a white solid precipitate was obtained. The precipitate was filtered and the filtrate was concentrated to dryness to afford 2-(4-benzylpiperazin-1-yl)propan-1-ol (3.27 g, 99%) as a yellow oil. A mixture of palladium hydroxide 20% on carbon (0.575 g, 4.10 mmol), cyclohexene (34 ml) and 2-(4-benzylpiperazin-1-yl)propan-1-ol (3.2 g, 13.6 mmol) in ethanol (60 ml) was stirred at 80° C. for 3 hours. After cooling the catalyst was filtered on celite and the filtrate was concentrated to dryness to afford 2-(piperazin-1-yl)propan-1-ol (2.0 g, 100%) as a brown oil. This product was used without further purification. NMR Spectrum: (CDCl₃) 0.91 (d, 3H), 2.32 (bs, 2H), 2.34-2.44 (m, 2H), 2.59-2.68 (m, 2H), 2.73-2.82 (m, 1H), 2.83-2.98 (m, 4H), 3.32 (dd, 1H), 3.40 (dd, 1H)

### Example 275

### 1-[4-[4-[(5-chloro-4-imidazo[1,2-a]pyridin-3-yl-pyrimidin-2-yl)amino]-3-methoxy-phenyl]piperazin-1-yl]-2-morpholino-ethanone

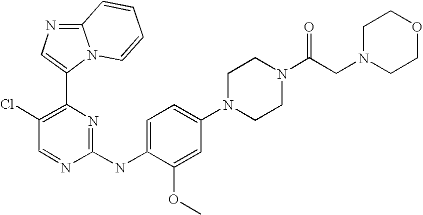

A mixture of 5-chloro-4-(imidazo[1,2-a]pyridin-3-yl)-N-(2-methoxy-4-(piperazin-1-yl)phenyl)pyrimidin-2-amine (Example 2, 110 mg, 0.25 mmol), 2-morpholinoacetic acid (Journal of Medicinal Chemistry 2004, p. 6935, 37 mg, 0.25 mmol), N-ethyl-N-isopropylpropan-2-amine (0.088 ml, 0.50 mmol) and 2-(3H-[1,2,3]triazolo[4,5-b]pyridin-3-yl)-1,1,3,3-tetramethylisouronium hexafluorophosphate(V) (173 mg, 0.45 mmol) in DMA (1 ml) was stirred at 25° C. for 2 hours. The reaction mixture was purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent to afford 1-(4-(4-(5-chloro-4-(imidazo[1,2-a]pyridin-3-yl)pyrimidin-2-ylamino)-3-methoxyphenyl)piperazin-1-yl)-2-morpholinoethanone (100 mg, 70%) as a beige solid; NMR spectrum: 2.43 (bs, 4H), 3.13-3.18 (m, 2H), 3.19-3.27 (m, 2H), 3.56-3.61 (m, 4H), 3.61-3.66 (m, 4H), 3.70-3.77 (m, 2H), 3.77 (s, 3H), 6.57 (dd, 1H), 6.75 d, 1H), 6.89 (bs, 1H), 7.30 (d, 1H), 7.48 (dd, 1H), 7.76 (d, 1H), 8.46 (s, 1H), 8.72 (s, 1H), 8.81 (s, 1H), 9.55 (bs, 1H); Mass spectrum: 563 (MH+).

### Example 276

### N-[2-[4-[(5-chloro-4-imidazo[1,2-a]pyridin-3-yl-pyrimidin-2-yl)amino]-3-methoxy-phenyl]-3-hydroxy-propyl]acetamide

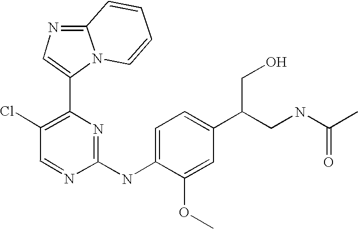

Sodium hydride (1.028 g, 25.7 mmol) was added to a stirred solution of methyl 2-cyanoacetate (1.03 ml, 11.7 mmol) in DMF (30 ml) at room temperature. The solution was stirred at room temperature for one hour then 4-fluoro-2-methoxy-1-nitrobenzene (2 g, 11.7 mmol) was added. The reaction was stirred at 100° C. for 4 hours then quenched with a saturated solution of ammonium chloride (30 ml) and extracted with ethyl acetate. The organic phase was washed with water then brine, dried over magnesium sulfate and concentrated. The crude product was purified by flash chromatography on silica gel eluting with 0 to 30% EtOAc/petroleum ether to afford methyl 2-cyano-2-(3-methoxy-4-nitrophenyl)acetate (2.3 g, 79%).

Borane methyl sulfide complex (4.48 mL, 8.96 mmol) was added dropwise to a stirred solution of methyl 2-cyano-2-(3-methoxy-4-nitrophenyl)acetate (0.83 g, 2.99 mmol) dissolved in THF (12 mL). The reaction mixture was heated at 85° C. under argon for 16 hours. After cooling to 0° C., 8 ml of saturated aqueous NaHCO3 was added. The mixture was extracted with EtOAc, then 10% MeOH/EtOAc. The combined the organic layers were dried over MgSO4 and evaporated. The crude product was purified by flash chromatography on silica gel eluting with 2 to 15% MeOH (7N NH3) in methylene chloride to afford 3-amino-2-(3-methoxy-4-nitrophenyl)propan-1-ol (0.200 g, 29%)

A mixture of 1,1′-(2-oxo-1H-benzo[d]imidazole-1,3(2H)-diyl)diethanone (193 mg, 0.88 mmol) and 3-amino-2-(3-methoxy-4-nitrophenyl)propan-1-ol (200 mg, 0.88 mmol) in THF (15 mL) was stirred under argon at 85° C. for 5 hours. After filtration, the filtrate was evaporated and the crude product was purified by flash chromatography on silica gel to afford N-(3-hydroxy-2-(3-methoxy-4-nitrophenyl)propyl)acetamide (197 mg, 83%).

A solution of N-(3-hydroxy-2-(3-methoxy-4-nitrophenyl)propyl)acetamide (197 mg, 0.73 mmol) in ethanol (4 ml) was hydrogenated under 1 atm at 25° C. for 2 hours in the presence of platinum(IV) oxide (34 mg). The mixture was filtered and the filtrate was concentrated to dryness to afford N-(2-(4-amino-3-methoxyphenyl)-3-hydroxypropyl)acetamide (140 mg, 80%) as a colorless oil. NMR Spectrum: 1.75 (s, 3H), 2.64-2.72 (m, 1H), 3.32-3.41 (bs partially hidden by H2O, 1H), 3.44-3.52 (m, 2H), 3.74 (s, 3H), 4.49 (bs, 2H), 4.56 (t, 1H), 6.50 (dd, 1H), 6.54 (d, 1H), 6.63 (d, 1H), 7.67 (t, 1H).

A mixture of 3-(2,5-dichloropyrimidin-4-yl)imidazo[1,2-a]pyridine (Method 1, 150 mg, 0.54 mmol), N-(2-(4-amino-3-methoxyphenyl)-3-hydroxypropyl)acetamide (135 mg, 0.54 mmol) and pTsOH (102 mg, 0.54 mmol) in 2-pentanol (1.5 mL) was heated at 140° C. for 2 hours.

The reaction mixture was diluted with methylene chloride, washed with a saturated solution of sodium bicarbonate, brine, dried over MgSO4, filtered and evaporated to dryness. The crude product was purified by flash chromatography on silica gel eluting with 2 to 10% MeOH/CH₂Cl₂ to afford the expected product which was further purified by preparative HPLC using a Waters X-Bridge reverse-phase column (5 microns silica, 30 mm diameter, 150 mm length) and decreasingly polar mixtures of water (containing 0.2% ammonium carbonate) and acetonitrile as eluent. The fractions were evaporated to dryness to afford after trituration in ether/pentane N-(2-(4-(5-chloro-4-(imidazo[1,2-a]pyridin-3-yl)pyrimidin-2-ylamino)-3-methoxyphenyl)-3-hydroxypropyl)acetamide (20 mg, 8%); NMR spectrum: 1.77 (s, 3H), 2.85-2.95 (m, 1H), 3.33-3.40 (m partially hidden by H2O, 1H), 3.40-3.47 (m, 1H), 3.57-3.66 (m, 2H), 3.78 (s, 3H), 4.72 (t, 1H), 6.84 (d, 1H), 6.99 (s, 1H), 7.00 (d, 1H), 7.44-7.52 (m, 2H), 7.76 (d, 1H), 7.82 (dd, 1H), 8.51 (s, 1H), 8.74 (s, 1H), 8.89 (s, 1H), 9.62 (bs, 1H); Mass spectrum: 467 (MH+).

### Example 277

### 1-[3-[4-[(5-chloro-4-imidazo[1,2-a]pyridin-3-yl-pyrimidin-2-yl)amino]-3-methoxy-phenyl]azetidin-1-yl]ethanone

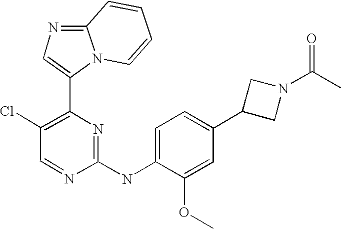

To a solution of 2-(3-methoxy-4-nitrophenyl)propane-1,3-diol (see Method 38, 0.454 g, 2.00 mmol) in dry acetonitrile (6 ml) at −40° C. was slowly added trifluoromethanesulfonic anhydride (0.59 ml, 3.5 mmol) over 10 min followed by N-ethyl-N-isopropylpropan-2-amine (1.00 ml, 5.75 mmol) over 10 min. Both reagents were added at such a rate as to maintain the temperature below −30° C., and the reaction mixture was aged for 20 min at −40 to −30° C. Additional N-ethyl-N-isopropylpropan-2-amine (1.00 ml, 5.75 mmol) was then added over 5 min at −40° C. followed by diphenylmethanamine (0.273 ml, 1.58 mmol) over 5 min and the reaction was heated to 70° C. for 1 h30. The reaction mixture was concentrated to dryness and the crude product was purified by flash chromatography on silica gel eluting with 0 to 30% EtOAc/petroleum ether. The solvent was evaporated to dryness to afford 1-benzhydryl-3-(3-methoxy-4-nitrophenyl)azetidine (80 mg, 7%).

A mixture of 1-benzhydryl-3-(3-methoxy-4-nitrophenyl)azetidine (240 mg, 0.64 mmol), palladium/C 10% (68 mg, 0.64 mmol), hydrogen chloride 5N in iPrOH (0.128 mL, 0.64 mmol) in methanol (3 mL) was hydrogenated at 60 psi for 5 hours. The reaction mixture was filtered through celite and evaporated to dryness. The crude product was purified by flash chromatography on silica gel eluting with 0 to 100% EtOAc/petroleum ether to afford 4-(1-benzhydrylazetidin-3-yl)-2-methoxyaniline (175 mg, 79%) as a colorless oil. This product was dissolved in MeOH (3 mL) and concentrated hydrochloric acid (0.053 mL, 0.64 mmol) was added. This mixture was hydrogenated under 60 psi at 25° C. for 5 hours. The resulting suspension was taken-up into MeOH/NH3 and concentrated to dryness. The crude product was purified by flash chromatography on silica gel eluting with 2 to 20% MeOH/CH₂Cl₂ to afford 4-(azetidin-3-yl)-2-methoxyaniline (115 mg, 100%) as a colorless oil. This product was dissolved in methylene chloride (3 mL) and acetic anhydride (0.36 ml, 3.23 mmol) was added. The resulting suspension was stirred for 3 hours. After filtration and evaporation of the filtrate, the residue was taken in H2O/EtOAc, the pH was adjusted to 6 with 2N NaOH and the aqueous layer was back extracted with EtOAc. The combined organic extracts were washed with brine, dried over MgSO4, filtered and concentrated to afford N-(4-(1-acetylazetidin-3-yl)-2-methoxyphenyl)acetamide as a pale yellow oil (128 mg, 78%). Sodium hydride (31 mg, 0.77 mmol) was added to a stirred suspension of N-(4-(1-acetylazetidin-3-yl)-2-methoxyphenyl)acetamide (85 mg, 0.32 mmol) in THF (4 mL) at room temperature under nitrogen. The resulting mixture was stirred for 35 minutes then 3-(2,5-dichloropyrimidin-4-yl)imidazo[1,2-a]pyridine (Method 1, 90 mg, 0.32 mmol) was added. The resulting mixture was stirred at 60° C. 1 hour. After cooling, methanol (10 ml), water (0.5 mL) and 10M NaOH (0.75 mL) were added, the solution was stirred at 25° C. for 1 hour. After evaporation, water was added and the mixture was extracted with ethyl acetate. The organic phase was washed with brine, dried and evaporated. The crude product was purified by flash chromatography on silica gel eluting with 0 to 10% MeOH/CH₂Cl₂ containing 50% EtOAc. The solvent was evaporated to dryness to afford 1-(3-(4-(5-chloro-4-(imidazo[1,2-a]pyridin-3-yl)pyrimidin-2-ylamino)-3-methoxyphenyl)azetidin-1-yl)ethanone (60 mg, 41%); NMR spectrum: 1.82 (s, 3H), 3.83 (s, 3H), 3.84-3.95 (m, 2H), 4.14-4.22 (m, 1H), 4.22-4.32 (m, 1H), 4.46-4.59 (m, 1H), 6.94 (dd, 1H), 6.99 (d, 1H), 7.12 (s, 1H), 7.50 (dd, 1H), 7.60 (d, 1H), 7.77 (d, 1H), 8.52 (s, 1H), 8.73 (s, 1H), 8.93 (s, 1H), 9.61 (bs, 1H); Mass spectrum: 449 (MH+).

### Example 278

### 1-[4-[4-[(5-chloro-4-imidazo[1,2-a]pyridin-3-yl-pyrimidin-2-yl)amino]-5-methoxy-2-methyl-phenyl]piperazin-1-yl]ethanone

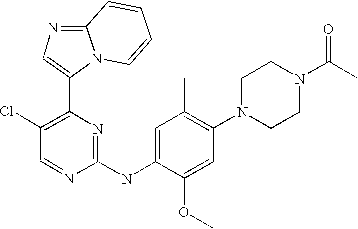

The title compound was prepared according to Example 1 using 1-(4-(4-amino-5-methoxy-2-methylphenyl)piperazin-1-yl)ethanone instead of 1-(4-(4-amino-3-methoxyphenyl)piperazin-1-yl)ethanone; NMR spectrum: 2.07 (s, 3H), 2.21 (s, 3H), 2.82-2.89 (m, 2H), 2.89-2.96 (m, 2H), 3.57-3.59 (m, 4H), 3.76 (s, 3H), 6.81 (dd, 1H), 7.34 (dd, 1H), 7.51 (ddd, 1H), 7.77 (d, 1H), 8.50 (s, 1H), 8.73 (s, 1H), 8.84 (s, 1H), 9.56 (bs, 1H); Mass spectrum: 492 (MH+).

The starting 1-(4-(4-amino-5-methoxy-2-methylphenyl)piperazin-1-yl)ethanone was prepared as follows:

Potassium carbonate (1.73 g, 12.5 mmol) was added in one portion to a stirred solution of 1-chloro-5-fluoro-4-methyl-2-nitrobenzene (1.90 g, 10.0 mmol) and 1-(piperazin-1-yl)ethanone (1.60 g, 12.5 mmol) in DMA (20 mL). The resulting suspension was stirred at 60° C. for 24 hours then concentrated and the orange mixture was taken up with water and extracted with ethyl acetate. The organic solution was dried over MgSO4, filtered and concentrated. The residue was purified by flash chromatography on silica gel eluting with 0 to 20% diethylether in methylene chloride to afford a yellow solid (2.4 g) which was taken up in ethanol (5 ml) and triturated to allow crystallisation. The suspension was diluted with diethylether (15 ml), stirred 5 min, filtered and the solid washed with ether and dried under vacuum at 50° C. to afford 1-(4-(5-chloro-2-methyl-4-nitrophenyl)piperazin-1-yl)ethanone (2.0 g, 67%) as a yellow solid.

Methanol (0.486 mL, 12.0 mmol) was added dropwise to a stirred suspension of sodium hydride (60% in oil, 448 mg, 11.2 mmol) in THF (25 mL) under nitrogen at 5° C. The suspension was stirred for 30 min, then 1,4,7,10,13-pentaoxacyclopentadecane (15-crown-5) (0.079 mL, 0.40 mmol) was added and the resulting mixture was added to a suspension of 1-(4-(5-chloro-2-methyl-4-nitrophenyl)piperazin-1-yl)ethanone (1190 mg, 4.00 mmol) in THF (15 mL). The resulting mixture was refluxed for 6 hours then poured onto an aqueous saturated solution of NH4Cl, then extracted with ethylacetate. The organic solution was dried over MgSO4, filtered and concentrated to afford a yellow solid. The crude material was purified by flash chromatography on silica gel eluting with 0 to 4% methanol in methylene chloride to afford 1-(4-(5-methoxy-2-methyl-4-nitrophenyl)piperazin-1-yl)ethanone (1.08 g, 92%) as a yellow solid.

Platinum(IV) oxide (0.131 g, 0.58 mmol) was added to a solution of 1-(4-(5-methoxy-2-methyl-4-nitrophenyl)piperazin-1-yl)ethanone (1.3 g, 4.43 mmol) in EtOAc (25 mL) and EtOH (25 mL). The mixture was hydrogenated under 1300 mbar at room temp for 2 hours. The resulting suspension was filtered, the filtrate was concentrated and the oil was taken up with diethylether (10 ml) to induce crystallisation. Petroleum ether was added the solid was triturated then filtered and dried under vacuum at 50° C. to afford 1-(4-(4-amino-5-methoxy-2-methylphenyl)piperazin-1-yl)ethanone (1.10 g, 94%) as a solid; NMR Spectrum: 2.03 (s, 3H), 2.10 (s, 3H), 2.64-2.69 (m, 2H), 2.70-2.76 (m, 2H), 3.48-3.58 (m, 4H), 3.71 (s, 3H), 4.36 (bs, 2H), 6.45 (s, 1H), 6.56 (s, 1H).

### Example 279

### 2-[4-[4-[(5-chloro-4-imidazo[1,2-a]pyridin-3-yl-pyrimidin-2-yl)amino]-5-methoxy-2-methyl-phenyl]piperazin-1-yl]ethanol

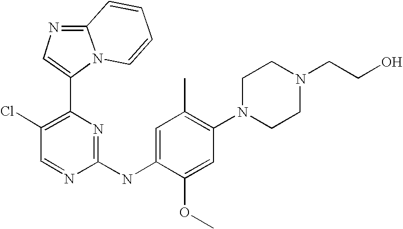

The title compound was prepared following the procedure described for Ex ample 278 using 2-(piperazin-1-yl)ethanol in the first step instead of 1-(piperazin-1-yl)ethanone; NMR spectrum: 2.18 (s, 3H), 2.48 (t, 2H), 2.57-2.67 (m, 4H), 2.85-2.95 (m, 4H), 3.53-3.56 (m, 2H), 3.77 (s, 3H), 4.44 (t, 1H), 6.80 (s, 1H), 6.89 (dd, 1H), 7.29 (s, 1H), 7.51 (ddd, 1H), 7.77 (d, 1H), 8.49 (s, 1H), 8.72 (s, 1H), 8.83 (s, 1H), 9.55 (bs, 1H); Mass spectrum: 494 (MH+).

### Example 280

### 1-[4-[4-[(5-chloro-4-imidazo[1,2-a]pyridin-3-yl-pyrimidin-2-yl)amino]-3-methoxy-2-methyl-phenyl]piperazin-1-yl]ethanone

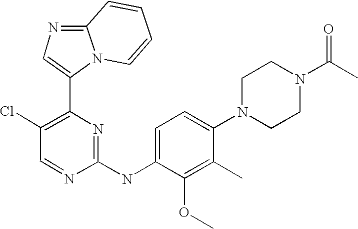

The title compound was prepared according to Example 1 using 1-(4-(4-amino-3-methoxy-2-methylphenyl)piperazin-1-yl)ethanone instead of 1-(4-(4-amino-3-methoxyphenyl)piperazin-1-yl)ethanone; NMR spectrum: 2.06 (s, 3H), 2.25 (s, 3H), 2.78-2.83 (m, 2H), 2.84-2.90 (m, 2H), 3.57-3.67 (m, 4H), 3.65 (s, 3H), 6.86 (s, 1H), 6.87 (s, 1H), 7.35 (d, 1H), 7.49 (dd, 1H), 7.76 (d, 1H), 8.51 (s, 1H), 8.72 (s, 1H), 9.08 (s, 1H), 9.57 (bs, 1H); Mass spectrum: 492 (MH+).

The starting 1-(4-(4-amino-3-methoxy-2-methylphenyl)piperazin-1-yl)ethanone was prepared as follows:

A mixture of 1,3-dichloro-2-methyl-4-nitrobenzene (2.09 g, 10.1 mmol), 1-(piperazin-1-yl)ethanone (1.30 g, 10.1 mmol) and cesium carbonate (3.64 g, 11.1 mmol) in DMA (30 mL) was stirred at 120° C. for 16 hours. The reaction mixture was diluted with a saturated aqueous solution of sodium hydrogencarbonate and extracted with diethyl ether (2×100 ml). The combined organic phases were washed with water, dried over magnesium sulfate and concentrated. The crude product was purified by flash chromatography on silica gel eluting with petroleum ether and methylene chloride to afford 1-(4-(3-chloro-2-methyl-4-nitrophenyl)piperazin-1-yl)ethanone (0.240 g, 8%).

Methanol (0.045 mL, 1.11 mmol) was added dropwise to a stirred suspension of sodium hydride (44 mg, 1.11 mmol) in DMA (1 mL) under nitrogen. The resulting suspension was stirred at 0° C. for 30 min then a solution of 1-(4-(3-chloro-2-methyl-4-nitrophenyl)piperazin-1-yl)ethanone (220 mg, 0.74 mmol) in DMA (2 mL) was added dropwise and the resulting mixture was stirred at 60° C. for 24 hours. The reaction mixture was concentrated and the residue taken up with water and extracted with ethyl acetate. The organic solution was dried over MgSO4, filtered and concentrated. The residue was purified by flash chromatography on silica gel eluting with 0 to 10% diethylether in methylene chloride to afford 1-(4-(3-methoxy-2-methyl-4-nitrophenyl)piperazin-1-yl)ethanone (140 mg, 64%) as a yellow solid. Platinum(IV) oxide (16.2 mg, 0.07 mmol) was added in one portion to a stirred solution of 1-(4-(3-methoxy-2-methyl-4-nitrophenyl)piperazin-1-yl)ethanone (140 mg, 0.48 mmol) in ethyl acetate (10 mL) and ethanol (10 mL) and the resulting suspension was hydrogenated under 1300 mbar at room temperature for 2 hours. The suspension was filtered and the filtrate was concentrated to dryness to afford the crude 1-(4-(4-amino-3-methoxy-2-methylphenyl)piperazin-1-yl)ethanone (120 mg, 95%) which was used without further purification. NMR Spectrum: 2.02 (s, 3H), 2.14 (s, 3H), 2.58-2.66 (m, 2H), 2.66-2.73 (m, 2H), 3.51 (bs, 4H), 3.59 (s, 3H), 4.56 (s, 2H), 6.49 (d, 1H), 6.58 (d, 1H).

### Example 281

### 1-[4-[4-[(5-chloro-4-imidazo[1,2-a]pyridin-3-yl-pyrimidin-2-yl)amino]-2,3-dimethoxy-phenyl]piperazin-1-yl]ethanone

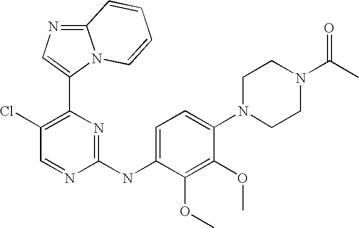

The title compound was prepared according to Example 1 using 1-(4-(4-amino-2,3-dimethoxyphenyl)piperazin-1-yl)ethanone instead of 1-(4-(4-amino-3-methoxyphenyl)piperazin-1-yl)ethanone; NMR spectrum: 2.06 (s, 3H), 2.94-3.02 (m, 2H), 3.02-3.09 (m, 2H), 3.59-3.67 (m, 4H), 3.74 (s, 3H), 3.83 (s, 3H), 6.74 (d, 1H), 6.89 (dd, 1H), 7.27 (d, 1H), 7.50 (ddd, 1H), 7.77 (d, 1H), 8.51 (s, 1H), 8.74 (s, 1H), 9.01 (s, 1H), 9.59 (bs, 1H); Mass spectrum: 508 (MH+).

The starting 1-(4-(4-amino-2,3-dimethoxyphenyl)piperazin-1-yl)ethanone was prepared as follows:

A mixture of 2,3-difluoro-6-nitrophenol (2.5 g, 14.3 mmol), potassium carbonate (1.97 g, 14.3 mmol) and iodomethane (3.56 ml, 57.1 mmol) in acetone was stirred at 60° C. for 24 hours. Acetone was evaporated and the residue was diluted in water and extracted with methylene chloride. The organic phase was dried over magnesium sulfate and concentrated and the crude product was purified by flash chromatography on silica gel eluting with 0 to 5% ethyl acetate in petroleum ether. The solvent was evaporated to dryness to afford 1,2-difluoro-3-methoxy-4-nitrobenzene (2.40 g, 89%) as a colorless liquid.

A mixture of 1,2-difluoro-3-methoxy-4-nitrobenzene (1.3 g, 6.87 mmol), 1-(piperazin-1-yl)ethanone (1.05 g, 8.25 mmol) and cesium carbonate (2.46 g, 7.56 mmol) in DMA (6 mL) was stirred at 25° C. overnight. The reaction mixture was diluted with water and the precipitate was collected by filtration and dried under vacuum to give 1-(4-(2-fluoro-3-methoxy-4-nitrophenyl)piperazin-1-yl)ethanone (1.81 g, 89%) as a yellow solid. Methanol (0.426 mL, 10.5 mmol) was added dropwise to a stirred suspension of sodium hydride (60% in oil, 0.420 g, 10.5 mmol) in DMA (10 mL) under nitrogen at 5° C. The resulting suspension was stirred for 30 min then a solution of 1-(4-(2-fluoro-3-methoxy-4-nitrophenyl)piperazin-1-yl)ethanone (1.04 g, 3.50 mmol) in DMA (10 mL) was added and the reaction mixture was stirred at 50° C. for 1 hour. The reaction mixture was concentrated and the residue was taken up in water and extracted with ethyl acetate. The organic solution was dried over MgSO₄, filtered and concentrated. The residue was purified by flash chromatography on silica gel eluting with 0 to 10% diethylether in methylene chloride to afford 1-(4-(2,3-dimethoxy-4-nitrophenyl)piperazin-1-yl)ethanone (0.135 g, 12%) as a yellow solid.

A mixture of platinum(IV) oxide (30 mg, 0.13 mmol) and 1-(4-(2,3-dimethoxy-4-nitrophenyl)piperazin-1-yl)ethanone (202 mg, 0.65 mmol) in EtOAc (10 mL) and EtOH (10 mL) was hydrogenated under 45 psi at room temperature for 1 hour. The suspension was filtered, the filtrate was concentrated and the oil was taken up with ethanol (2 ml) then petroleum ether was added to induce crystallisation. The solid was filtered and dried under vacuum at 40° C. to afford 1-(4-(4-amino-2,3-dimethoxyphenyl)piperazin-1-yl)ethanone (162 mg, 89%) as a solid; NMR Spectrum: 2.02 (s, 3H), 2.72-2.79 (m, 2H), 2.79-2.87 (m, 2H), 3.49-3.60 (m, 4H), 3.68 (s, 3H), 3.81 (s, 3H), 4.58 (bs, 2H), 6.35 (d, 1H), 6.47 (d, 1H).

### Example 282

### 1-[4-[4-[(5-chloro-4-imidazo[1,2-a]pyridin-3-yl-pyrimidin-2-yl)amino]-2-fluoro-3-methoxy-phenyl]piperazin-1-yl]ethanone

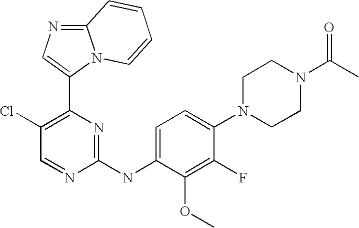

The title compound was prepared according to Example 1 using 1-[4-(4-amino-2-fluoro-3-methoxy-phenyl)piperazin-1-yl]ethanone instead of 1-(4-(4-amino-3-methoxyphenyl)piperazin-1-yl)ethanone; NMR spectrum: 2.06 (s, 3H), 2.93-3.01 (m, 2H), 3.01-3.08 (m, 2H), 3.57-3.70 (m, 4H), 3.79 (s, 3H), 6.82 (dd, 1H), 6.91 (dd, 1H), 7.29 (d, 1H), 7.51 (ddd, 1H), 7.77 (d, 1H), 8.52 (s, 1H), 8.73 (s, 1H), 9.19 (s, 1H), 9.60 (bs, 1H); Mass spectrum: 496 (MH+).

The starting 1-[4-(4-amino-2-fluoro-3-methoxy-phenyl)piperazin-1-yl]ethanone was prepared as follows:

A mixture of 1-(4-(2-fluoro-3-methoxy-4-nitrophenyl)piperazin-1-yl)ethanone (see Example 281, starting material, 1.5 g, 5.05 mmol) and platinum(IV) oxide (0.229 g, 1.01 mmol) in ethanol (20 mL) and EtOAc (20 mL) was hydrogenated under 1.2 bar at 25° C. for 16 hours. The mixture was filtered and evaporated to give 1-(4-(4-amino-2-fluoro-3-methoxyphenyl)piperazin-1-yl)ethanone (1.35 g, 100%) as a yellow solid. NMR Spectrum: 2.02 (s, 3H), 2.72-2.78 (m, 2H), 2.79-2.85 (m, 2H), 3.50-3.59 (m, 4H), 3.72 (s, 3H), 4.82 (bs, 2H), 6.39 (dd, 1H), 6.55 (dd, 1H).

### Example 283

### 6-(4-acetylpiperazin-1-yl)-3-[(5-chloro-4-imidazo[1,2-a]pyridin-3-yl-pyrimidin-2-yl)amino]-2-methoxy-benzonitrile

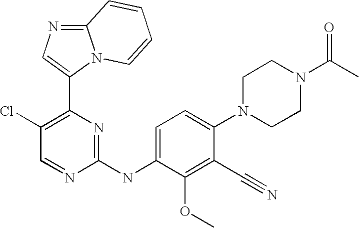

The title compound was prepared according to Example 1 using 6-(4-acetylpiperazin-1-yl)-3-amino-2-methoxybenzonitrile instead of 1-(4-(4-amino-3-methoxyphenyl)piperazin-1-yl)ethanone; NMR spectrum: 2.06 (s, 3H), 3.07-3.14 (m, 2H), 3.15-3.21 (m, 2H), 3.59-3.70 (m, 4H), 3.88 (s, 3H), 6.95 (d, 1H), 6.96 (bs, 1H), 7.53 (ddd, 1H), 7.71 (d, 1H), 7.78 (d, 1H), 8.54 (s, 1H), 8.72 (s, 1H), 9.39 (s, 1H), 9.57 (bs, 1H); Mass spectrum: 503 (MH+).

The starting 6-(4-acetylpiperazin-1-yl)-3-amino-2-methoxybenzonitrile was prepared as follows:

A mixture of 2,6-difluoro-3-nitrobenzonitrile (220 mg, 1.20 mmol), 1-(piperazin-1-yl)ethanone (153 mg, 1.20 mmol) and cesium carbonate (428 mg, 1.31 mmol) in DMA (3 mL) was stirred at 25° C. for 2 hours. The reaction mixture was diluted with water and the precipitate formed was collected by filtration and dried under vacuum to give 6-(4-acetylpiperazin-1-yl)-2-fluoro-3-nitrobenzonitrile (260 mg, 74%) as a yellow solid.

A mixture of 6-(4-acetylpiperazin-1-yl)-2-fluoro-3-nitrobenzonitrile (145 mg, 0.50 mmol) and cesium carbonate (178 mg, 0.55 mmol) in methanol (0.5 mL) was stirred at 25° C. for 1 hours. The reaction mixture was diluted with water and the aqueous layer was extracted with methylene chloride. The organic phase was dried over magnesium sulfate and concentrated to afford the crude 6-(4-acetylpiperazin-1-yl)-2-methoxy-3-nitrobenzonitrile (145 mg, 96%). A suspension of 6-(4-acetylpiperazin-1-yl)-2-methoxy-3-nitrobenzonitrile (200 mg, 0.66 mmol) and platinum(IV) oxide (60 mg, 0.26 mmol) in methanol (0.5 mL), EtOAc (2 mL) and EtOH (3 mL) was hydrogenated under 1600 mbar at 25° C. for 2 hours. The resulting suspension was filtered and the filtrate was concentrated to dryness to afford the crude 6-(4-acetylpiperazin-1-yl)-3-amino-2-methoxybenzonitrile (177 mg, 98%) which was used without further purification. NMR Spectrum: 2.03 (s, 3H), 2.82-2.87 (m, 2H), 2.88-2.93 (m, 2H), 3.52-3.59 (m, 4H), 3.78 (s, 3H), 5.09 (bs, 2H), 6.74 (d, 1H), 6.92 (d, 1H).

### Example 284

### 1-[4-[4-[(5-chloro-4-imidazo[1,2-a]pyridin-3-yl-pyrimidin-2-yl)amino]-2-fluoro-5-methoxy-phenyl]piperazin-1-yl]ethanone

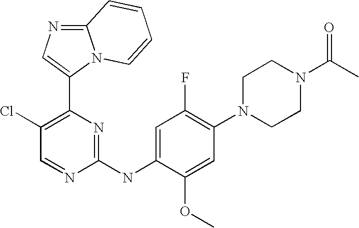

The title compound was prepared according to Example 1 using 1-(4-(4-amino-2-fluoro-5-methoxyphenyl)piperazin-1-yl)ethanone instead of 1-(4-(4-amino-3-methoxyphenyl)piperazin-1-yl)ethanone; NMR spectrum: 2.06 (s, 3H), 2.97-3.04 (m, 2H), 3.04-3.12 (m, 2H), 3.57-3.68 (m, 4H), 3.81 (s, 3H), 6.76 (d, 1H), 6.98 (dd, 1H), 7.52 (d, 1H), 7.54 (d, 1H), 7.78 (d, 1H), 8.54 (s, 1H), 8.73 (s, 1H), 8.90 (s, 1H), 9.67 (bs, 1H); Mass spectrum: 496 (MH+).

The starting 1-(4-(4-amino-2-fluoro-5-methoxyphenyl)piperazin-1-yl)ethanone was prepared as follows:

A mixture of 1,2,4-trifluoro-5-nitrobenzene (2.75 g, 15.5 mmol), 1-(piperazin-1-yl)ethanone (1.99 g, 15.5 mmol) and cesium carbonate (5.57 g, 17.1 mmol) in DMA (30 mL) was stirred at 25° C. for 16 hours. The reaction mixture was diluted with water and extracted with methylene chloride. The organic phase was dried over magnesium sulfate and concentrated to give a mixture of 1-(4-(2,5-difluoro-4-nitrophenyl)piperazin-1-yl)ethanone and 1-(4-(4,5-difluoro-2-nitrophenyl)piperazin-1-yl)ethanone (ratio 9:1, 5.85 g, 66%). This mixture of product (11.5 g, 15.5 mmol) was reacted with cesium carbonate (5.57 g, 17.1 mmol) in methanol (10 mL) at 25° C. for 16 hours. The reaction mixture was diluted with water and the resulting solid was collected by filtration and dried under vacuum. This crude product was purified by flash chromatography on silica gel eluting with 0 to 50% ethyl acetate in methylene chloride to afford 1-(4-(2-fluoro-5-methoxy-4-nitrophenyl)piperazin-1-yl)ethanone (1.55 g, 33%) as a yellow solid.

A suspension of 1-(4-(2-fluoro-5-methoxy-4-nitrophenyl)piperazin-1-yl)ethanone (1.5 g, 5.05 mmol) and platinum(IV) oxide (0.229 g, 1.01 mmol) in ethyl acetate (10 mL) and ethanol (30 mL) was hydrogenated under 1300 mbar at 25° C. for 3 hours. The mixture was filtered and the filtrate was concentrated to dryness to afford the crude 1-(4-(4-amino-2-fluoro-5-methoxyphenyl)piperazin-1-yl)ethanone (1.38 g, 100%); NMR Spectrum: 2.02 (s, 3H), 2.77-2.83 (m, 2H), 2.83-2.90 (m, 2H), 3.49-3.59 (m, 4H), 3.73 (s, 3H), 4.66 (bs, 2H), 6.43 (d, 1H), 6.55 (d, 1H).

### Example 285

### 1-[4-[4-[(5-chloro-4-imidazo[1,2-a]pyridin-3-yl-pyrimidin-2-yl)amino]-2,5-dimethoxy-phenyl]piperazin-1-yl]ethanone

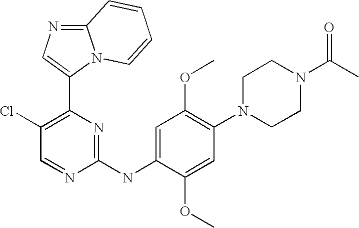

The title compound was prepared according to Example 1 using 1-(4-(4-amino-2,5-dimethoxyphenyl)piperazin-1-yl)ethanone instead of 1-(4-(4-amino-3-methoxyphenyl)piperazin-1-yl)ethanone; NMR spectrum: 2.05 (s, 3H), 2.93-3.01 (m, 2H), 3.01-3.08 (m, 2H), 3.56-3.66 (m, 4H), 3.69 (s, 3H), 3.74 (s, 3H), 6.70 (s, 1H), 6.88 (bs, 1H), 7.15 (s, 1H), 7.50 (dd, 1H), 7.76 (d, 1H), 8.49 (s, 1H), 8.72 (s, 1H), 8.89 (s, 1H), 9.60 (bs, 1H); Mass spectrum: 508 (MH+).

The starting 1-(4-(4-amino-2,5-dimethoxyphenyl)piperazin-1-yl)ethanone was prepared as follows:

A mixture of potassium carbonate (4.13 g, 29.9 mmol), 1-chloro-2,5-dimethoxy-4-nitrobenzene (4.34 g, 19.9 mmol) and 1-(piperazin-1-yl)ethanone (3.83 g, 29.9 mmol) in DMA (40 mL) was stirred at 160° C. for 24 hours under argon. The mixture was concentrated and the residue was taken up with water and extracted with ethyl acetate. The organic solution was dried over MgSO4, filtered and concentrated. The residue was purified by flash chromatography on silica gel eluting with 0 to 20% diethylether in methylene chloride to afford 1-(4-(2,5-dimethoxy-4-nitrophenyl)piperazin-1-yl)ethanone (850 mg, 13%).

A mixture of platinum(IV) oxide (57 mg, 0.25 mmol), 1-(4-(2,5-dimethoxy-4-nitrophenyl)piperazin-1-yl)ethanone (780 mg, 2.52 mmol) in EtOAc (20 mL) and EtOH (20 mL was hydrogenated under 1300 mbar at room temperature for 1 hour. The suspension was filtered, the filtrate was concentrated and the oil was taken up with ethanol. Petroleum ether was added to induce crystallisation. The solid was filtered and dried under vacuum at 40° C. to afford 1-(4-(4-amino-2,5-dimethoxyphenyl)piperazin-1-yl)ethanone (660 mg, 94%); NMR Spectrum: 2.01 (s, 3H), 2.74-2.80 (m, 2H), 2.81-2.86 (m, 2H), 3.48-3.55 (m, 4H), 3.67 (s, 3H), 3.68 (s, 3H), 4.43 (bs, 2H), 6.36 (s, 1H), 6.47 (s, 1H).

### Example 286

### 2-(4-acetylpiperazin-1-yl)-5-[(5-chloro-4-imidazo[1,2-a]pyridin-3-yl-pyrimidin-2-yl)amino]-4-methoxy-benzonitrile

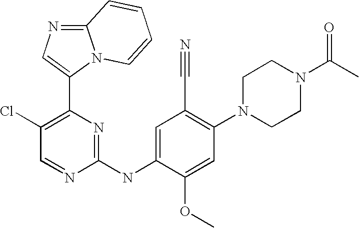

The title compound was prepared according to Example 1 using 2-(4-acetylpiperazin-1-yl)-5-amino-4-methoxybenzonitrile instead of 1-(4-(4-amino-3-methoxyphenyl)piperazin-1-yl)ethanone; NMR spectrum: 2.07 (s, 3H), 3.11-3.19 (m, 2H), 3.19-3.25 (m, 2H), 3.60-3.71 (m, 4H), 3.90 (s, 3H), 6.84 (s, 1H), 7.01 (dd, 1H), 7.54 (dd, 1H), 7.78 (d, 1H), 7.96 (s, 1H), 8.55 (s, 1H), 8.73 (s, 1H), 9.02 (s, 1H), 9.62 (bs, 1H); Mass spectrum: 503 (MH+).

The starting 2-(4-acetylpiperazin-1-yl)-5-amino-4-methoxybenzonitrile was prepared as follows:

2-fluoro-4-methoxybenzonitrile (2 g, 13.23 mmol) was added to a stirred mixture of potassium nitrate (1.47 g, 14.5 mmol) and concentrated sulfuric acid (13 mL) at 0° C. The reaction mixture was stirred at 0° C. for 1 hour then carefully poured onto a saturated aqueous solution of sodium hydrogencarbonate at 0° C. under stirring. The resulting mixture was extracted with methylene chloride and the combined organic phases were dried over magnesium sulfate and concentrated. The crude product was purified by flash chromatography on silica gel eluting with methylene chloride to afford 2-fluoro-4-methoxy-5-nitrobenzonitrile (2.26 g, 87%).

A mixture of 2-fluoro-4-methoxy-5-nitrobenzonitrile (2.2 g, 11.2 mmol), 1-(piperazin-1-yl)ethanone (1.58 g, 12.3 mmol) and cesium carbonate (4.02 g, 12.3 mmol) in DMA (20 mL) was stirred at 25° C. overnight. The reaction mixture was diluted with water and the precipitate formed was collected by filtration and dried under vacuum to give 2-(4-acetylpiperazin-1-yl)-4-methoxy-5-nitrobenzonitrile (2.84 g, 83%) as a yellow solid.

A mixture of 2-(4-acetylpiperazin-1-yl)-4-methoxy-5-nitrobenzonitrile (2.82 g, 9.27 mmol) and platinum(IV) oxide (0.210 g, 0.93 mmol) in ethyl acetate (10 mL) and ethanol (30 mL) was hydrogenated at 25° C. for 4 hours. The reaction mixture was filtered and the filtrate was evaporated. The crude product was purified by flash chromatography on silica gel eluting with 0 to 5% methanol in ethyl acetate to afford 2-(4-acetylpiperazin-1-yl)-5-amino-4-methoxybenzonitrile (2.28 g, 90%) as a white solid. NMR Spectrum: 2.04 (s, 3H), 2.87-2.94 (m, 2H), 2.94-3.01 (m, 2H), 3.54-3.62 (m, 4H), 3.84 (s, 3H), 4.85 (bs, 2H), 6.68 (s, 1H), 6.83 (s, 1H).

### Example 287

### 1-[4-[4-[(5-chloro-4-imidazo[1,2-a]pyridin-3-yl-pyrimidin-2-yl)amino]-3-(2-methoxyethoxy)phenyl]piperazin-1-yl]ethanone

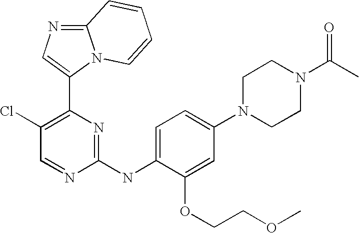

The title compound was prepared according to Example 1 using 1-(4-(4-amino-3-(2-methoxyethoxy)phenyl)piperazin-1-yl)ethanone instead of 1-(4-(4-amino-3-methoxyphenyl)piperazin-1-yl)ethanone; NMR spectrum: 2.07 (s, 3H), 3.10 (s, 3H), 3.12-3.17 (m, 2H), 3.18-3.23 (m, 2H), 3.48-3.53 (m, 2H), 3.58-3.64 (m, 4H), 4.07-4.12 (m, 2H), 6.58 (dd, 1H), 6.76 (d, 1H), 6.86 (bs, 1H), 7.34 (d, 1H), 7.49 (dd, 1H), 7.75 (d, 1H), 8.47 (s, 1H), 6.72 (s, 1H), 6.73 (s, 1H), 9.50 (bs, 1H); Mass spectrum: 522 (MH+).

The starting 1-(4-(4-amino-3-(2-methoxyethoxy)phenyl)piperazin-1-yl)ethanone was prepared as follows:

1-bromo-2-methoxyethane (1.19 mL, 12.7 mmol) was added to a stirred mixture of 5-fluoro-2-nitrophenol (2 g, 12.7 mmol) and potassium carbonate (4.40 g, 31.8 mmol) in DMF (35 mL). The resulting mixture was heated at 110° C. for 4 hours. The yellow reaction mixture was diluted with water and extracted with ethyl acetate. The organic layer was washed with a 1M aqueous solution of sodium hydroxide, water and brine, then dried over magnesium sulfate and concentrated to afford crude 4-fluoro-2-(2-methoxyethoxy)-1-nitrobenzene (2.50 g, 91%) as a yellow liquid.

A mixture of 1-(piperazin-1-yl)ethanone (1.46 g, 11.4 mmol), N,N-diisopropylethylamine (2.38 mL, 13.6 mmol) and 4-fluoro-2-(2-methoxyethoxy)-1-nitrobenzene (2.45 g, 11.4 mmol) in DMA (7 mL) was heated at 90° C. for 15 hours. After cooling water was added, the precipitate was stirred during 15 minutes then filtered, washed with water and dried in vacuum to afford 1-(4-(3-(2-methoxyethoxy)-4-nitrophenyl)piperazin-1-yl)ethanone (3.23 g, 88%) as a yellow solid.

A suspension of 1-(4-(3-(2-methoxyethoxy)-4-nitrophenyl)piperazin-1-yl)ethanone (3.2 g, 9.90 mmol) and platinum(IV) oxide (0.112 g, 0.49 mmol) in ethanol (220 ml) was hydrogenated under 1.3 bar at 25° C. for 3 hours. The resulting suspension was diluted with methylene chloride and filtered. The filtrate was concentrated and the residue purified by flash chromatography on silica gel eluting with 3 to 5% methanol in methylene chloride to afford 1-(4-(4-amino-3-(2-methoxyethoxy)phenyl)piperazin-1-yl)ethanone (2.10 g, 72%) as an oil which crystallised on standing. NMR Spectrum: 2.02 (s, 3H), 2.82-2.89 (m, 2H), 2.89-2.97 (m, 2H), 3.32 (s, 3H), 3.49-3.59 (m, 4H), 3.62-3.68 (m, 2H), 4.03-4.08 (m, 2H), 4.40 (bs, 2H), 6.34 (dd, 1H), 6.55 (d, 1H), 6.56 (d, 1H).

### Example 288

### 5-(4-acetylpiperazin-1-yl)-2-[(5-chloro-4-imidazo[1,2-a]pyridin-3-yl-pyrimidin-2-yl)amino]benzonitrile

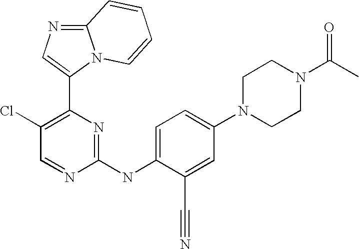

A mixture of 1-acetylpiperazine (424 mg, 3.31 mmol), N,N-diisopropylethylamine (0.629 mL, 3.61 mmol) and 5-fluoro-2-nitrobenzonitrile (500 mg, 3.01 mmol) in DMA (2 mL) was stirred at 105° C. for 16 hours. The reaction mixture was allowed to cool to room temperature and diluted with water. The precipitate was filtered, washed with water and dried over P2O5 under vacuum to afford 5-(4-acetylpiperazin-1-yl)-2-nitrobenzonitrile (813 mg, 98%) as yellow solid.

A suspension of 5-(4-acetylpiperazin-1-yl)-2-nitrobenzonitrile (400 mg, 1.46 mmol) and palladium 10% on carbon (46 mg, 0.44 mmol) in EtOH (40 ml), was hydrogenated under 1 bar at 25° C. for 3 hours. The resulting suspension was filtered and the filtrate was concentrated to dryness to afford 5-(4-acetylpiperazin-1-yl)-2-aminobenzonitrile (351 mg, 99%). NMR Spectrum: 2.02 (s, 3H), 2.84-2.91 (m, 2H), 2.91-2.98 (m, 2H), 3.49-3.57 (m, 4H), 5.57 (bs, 2H), 6.75 (d, 1H), 6.89 (d, 1H), 7.13 (dd, 1H)

A mixture of 3-(2,5-dichloropyrimidin-4-yl)imidazo[1,2-a]pyridine (Method 1, 150 mg, 0.57 mmol), 5-(4-acetylpiperazin-1-yl)-2-aminobenzonitrile (138 mg, 0.57 mmol), cesium carbonate (369 mg, 1.13 mmol), tris(dibenzylideneacetone)dipalladium (20.7 mg, 0.02 mmol) and 9,9-dimethyl-4,5-bis(diphenylphosphino)xanthene (26 mg, 0.045 mmol) in degassed 1,4-dioxane (2 ml) was sealed into a microwave tube and heated at 90° C. for 1 h. After cooling the suspension was diluted with water and extracted with ethyl acetate. The organic phase was filtered and the filtrate was washed with brine, dried over magnesium sulfate and concentrated. The residue was purified by flash chromatography on silica gel eluting with 5 to 9% methanol in a 60/40 mixture of CH₂Cl₂ and AcOEt. The solvent was evaporated to dryness to afford 5-(4-acetylpiperazin-1-yl)-2-(5-chloro-4-(imidazo[1,2-a]pyridin-3-yl)pyrimidin-2-ylamino)benzonitrile (40 mg, 15%); NMR spectrum: 2.06 (s, 3H), 3.19-3.25 (m, 2H), 3.26-3.31 (m, 2H), 3.56-3.68 (m, 4H), 6.92 (bs, 1H), 7.37 (dd, 1H), 7.42 (d, 1H), 7.46 (d, 1H), 7.52 (ddd, 1H), 7.79 (d, 1H), 8.54 (s, 1H), 8.78 (s, 1H), 9.54 (bs, 1H), 9.73 (s, 1H); Mass spectrum: 473 (MH+).

### Example 289

### 1-[4-[4-[(5-chloro-4-imidazo[1,2-a]pyridin-3-yl-pyrimidin-2-yl)amino]-3-(hydroxymethyl)phenyl]piperazin-1-yl]ethanone

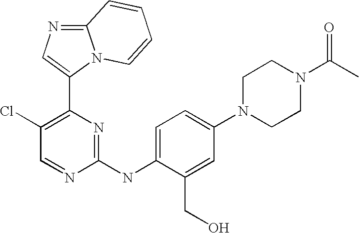

The title compound was prepared according to Example 288 using 1-(4-(4-amino-3-(hydroxymethyl)phenyl)piperazin-1-yl)ethanone instead of 5-(4-acetylpiperazin-1-yl)-2-aminobenzonitrile; NMR spectrum: 2.07 (s, 3H), 3.10-3.17 (m, 2H), 3.17-3.23 (m, 2H), 3.58-3.66 (m, 4H), 4.49 (d, 2H), 5.16 (t, 1H), 6.80 (bs, 1H), 6.94 (dd, 1H), 7.13 (d, 1H), 7.32 (d, 1H), 7.49 (dd, 1H), 7.75 (d, 1H), 8.47 (s, 1H), 8.74 (s, 1H), 9.00 (s, 1H), 9.44 (bs, 1H); Mass spectrum: 478 (MH+).

The starting 1-(4-(4-amino-3-(hydroxymethyl)phenyl)piperazin-1-yl)ethanone was prepared as follows:

Borane tetrahydrofuran complex (61.9 ml, 61.8 mmol) was added dropwise to a stirred solution of 5-fluoro-2-nitrobenzoic acid (4.58 g, 24.7 mmol) in THF (50 ml) over a period of 30 minutes at 0° C. under argon. The resulting solution was heated at 80° C. for 4 hours. After cooling at 0° C., methanol was added, the mixture was concentrated and water was added. The aqueous layer was extracted with ethyl acetate. The combined organic layers were washed with water, brine, dried over magnesium sulfate and concentrated to afford (5-fluoro-2-nitrophenyl)methanol (4.10 g, 97%).

A mixture of 1-acetylpiperazine (0.786 g, 6.14 mmol), N,N-diisopropylethylamine (1.22 mL, 7.01 mmol) and (5-fluoro-2-nitrophenyl)methanol (1 g, 5.84 mmol) in DMA (4 mL) was heated at 90° C. for 16 hours. The reaction mixture was allowed to cool to room temperature and water was added. The precipitate was filtered, washed with water and dried over P2O5 under vacuum to afford 1-(4-(3-(hydroxymethyl)-4-nitrophenyl)piperazin-1-yl)ethanone (1.45 g, 89%) as a yellow solid.

A suspension of 1-(4-(3-(hydroxymethyl)-4-nitrophenyl)piperazin-1-yl)ethanone (600 mg, 2.15 mmol) and platinum(IV) oxide (34 mg, 0.15 mmol) in EtOH (50 ml) was hydrogenated under 1.2 bar at 25° C. for 2 hours. The suspension was filtered, the filtrate was concentrated and the residue purified by flash chromatography on silica gel eluting with 5 to 15% methanol in methylene chloride. The solvent was evaporated to dryness to afford 1-(4-(4-amino-3-(hydroxymethyl)phenyl)piperazin-1-yl)ethanone (450 mg, 84%) as a pale yellow solid. NMR Spectrum: 2.02 (s, 3H), 2.80-2.87 (m, 2H), 2.87-2.94 (m, 2H), 3.49-3.57 (m, 4H), 4.35 (d, 2H), 4.51 (bs, 2H), 4.96 (t, 1H), 6.55 (d, 1H), 6.65 (dd, 1H), 6.78 (d, 1H).

### Example 290

### 1-[4-[4-[(5-chloro-4-imidazo[1,2-a]pyridin-3-yl-pyrimidin-2-yl)amino]-3-(methoxymethyl)phenyl]piperazin-1-yl]ethanone

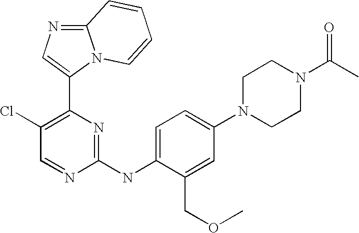

A mixture of 1-(4-(3-(hydroxymethyl)-4-nitrophenyl)piperazin-1-yl)ethanone (See Example 289, starting material, 2.5 g, 8.95 mmol), dimethyl sulfate (1.10 mL, 11.6 mmol), 50% aqueous KOH (15 mL) and methylene chloride (60 mL) was stirred at room temperature for 4 hours. The reaction mixture was quenched with water and extracted with methylene chloride. The combined organic phases were dried over magnesium sulfate and concentrated to dryness to afford 1-(4-(3-(methoxymethyl)-4-nitrophenyl)piperazin-1-yl)ethanone (2.60 g, 99%) as a yellow solid.

A suspension of 1-(4-(3-(methoxymethyl)-4-nitrophenyl)piperazin-1-yl)ethanone (2.6 g, 8.86 mmol) and platinum(IV) oxide (0.141 g, 0.62 mmol) in EtOH (140 ml) was hydrogenated under 1.3 bar at 2° C. for 2 hours. The suspension was filtered, the filtrate was concentrated and the residue purified by flash chromatography on silica gel eluting with 3% methanol in methylene chloride. The solvent was evaporated to dryness to afford 1-(4-(4-amino-3-(methoxymethyl)phenyl)piperazin-1-yl)ethanone (1.73 g, 74%) as a pale yellow solid. 1-(4-(4-amino-3-(methoxymethyl)phenyl)piperazin-1-yl)ethanone (150 mg, 0.57 mmol) was dissolved in a 1:1 mixture of acetic acid and acetic anhydride and the mixture was stirred at room temperature for 1 hour. The solution was concentrated to dryness and the resulting oil was purified by flash chromatography on silica gel eluting with 3 to 4% methanol in methylene chloride to afford N-(4-(4-acetylpiperazin-1-yl)-2-(methoxymethyl)phenyl)acetamide (150 mg, 86%) as a yellow solid.

Sodium hydride (39 mg, 0.97 mmol) was added to a stirred suspension of N-(4-(4-acetylpiperazin-1-yl)-2-(methoxymethyl)phenyl)acetamide (137 mg, 0.45 mmol) in THF (5 ml) at room temperature under nitrogen. The mixture was stirred at room temperature for 1 hour then 3-(2,5-dichloropyrimidin-4-yl)imidazo[1,2-a]pyridine (Method 1, 117 mg, 0.44 mmol) was added. The resulting mixture was stirred at 60° C. for 1 hour. After cooling 5 drops of 6N aqueous NaOH were added and the solution was heated at 60° C. for 1 hour. The solution was concentrated and the residue purified by preparative HPLC using a Waters X-Terra reverse-phase column (5 microns silica, 30 mm diameter, 150 mm length) and decreasingly polar mixtures of water (containing 0.2% ammonium carbonate) and acetonitrile as eluent to afford 1-(4-(4-(5-chloro-4-(imidazo[1,2-a]pyridin-3-yl)pyrimidin-2-ylamino)-3-(methoxymethyl)phenyl)piperazin-1-yl)ethanone (106 mg, 48%) as a yellow solid; NMR spectrum: 2.07 (s, 3H), 3.11-3.17 (m, 2H), 3.17-3.23 (m, 2H), 3.24 (s, 3H), 3.59-3.66 (m, 4H), 4.40 (s, 2H), 6.80 (bs, 1H), 6.99 (dd, 1H), 7.05 (d, 1H), 7.32 (d, 1H), 7.48 (dd, 1H), 7.75 (d, 1H), 8.47 (s, 1H), 8.75 (s, 1H), 8.99 (s, 1H), 9.43 (bs, 1H); Mass spectrum: 492 (MH+).

### Example 291

### 5-chloro-4-imidazo[1,2-a]pyridin-3-yl-N-[2-methoxy-4-(3-oxa-7,9-diazabicyclo[3.3.1]nonan-7-yl)phenyl]pyrimidin-2-amine

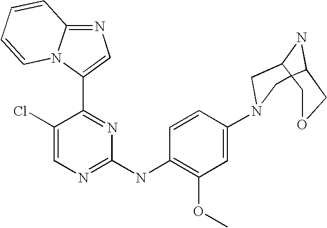

A mixture of 4-methylbenzenesulfonic acid hydrate (248 mg, 1.30 mmol), 4-(3-oxa-7,9-diazabicyclo[3.3.1]nonan-7-yl)-2-methoxyaniline (325 mg, 1.30 mmol) and 3-(2,5-dichloropyrimidin-4-yl)imidazo[1,2-a]pyridine (Method 1, 346 mg, 1.30 mmol) in 2-pentanol (3 mL) was heated in a microwave reactor at 140° C. for 1 hour. A saturated solution of sodium hydrogen carbonate (20 ml) was added and the mixture was extracted with methylene chloride. The organic extract was dried over MgSO4, filtered and evaporated. The residue was purified by flash chromatography on silica gel eluting with 1 to 3% of methanol in ethyl acetate to afford N-(4-(3-oxa-7,9-diazabicyclo[3.3.1]nonan-7-yl)-2-methoxyphenyl)-5-chloro-4-(imidazo[1,2-a]pyridin-3-yl)pyrimidin-2-amine (340 mg, 54%) as a yellow solid; NMR spectrum: 2.98 (bs, 2H), 3.04-3.11 (m, 2H), 3.75 (s, 3H), 3.75-3.88 (m, 6H), 6.44 (dd, 1H), 6.57 (d, 1H), 6.88 (bs, 1H), 7.21 (d, 1H), 7.44 (dd, 1H), 7.73 (d, 1H), 8.43 (s, 1H), 8.73 (s, 1H), 8.77 (s, 1H), 9.45 (bs, 1H); Mass spectrum: 478 (MH+).

The starting 4-(3-oxa-7,9-diazabicyclo[3.3.1]nonan-7-yl)-2-methoxyaniline was prepared as follows:

A solution of 3-(benzenesulfonyl)-9-benzyl-7-oxa-3,9-diazabicyclo[3.3.1]nonane (prepared as in Tetrahedron Letters 2005, vol. 46, p. 5577; 42 g, 0.12 mol) in concentrated HCl (250 mL) was stirred at reflux for 12 h. The reaction mixture was diluted with water, neutralized with solid NaOH until pH=7, then concentrated under reduced pressure to remove H₂O. The residue was poured into ethyl acetate, then being stirred at r.t. for 3 h. After filtration via Celite pad, the organic layer was concentrated under reduced pressure to dryness to provide 9-benzyl-7-oxa-3,9-diazabicyclo[3.3.1]nonane (13 g, 50%) as a brown oil; ¹H NMR: (MeOD, 400 MHz) 7.39-7.37 (m, 2H), 7.31-7.27 (m, 2H), 7.23-7.21 (m, 1H), 4.17-4.23 (m, 2H), 4.02 (s, 2H), 3.87-3.85 (d, J=8 Hz, 2H), 3.34-3.29 (m, 2H), 2.95-2.91 (d, J=16 Hz, 2H), 2.41 (s, 2H).

A mixture of 4-fluoro-2-methoxy-1-nitrobenzene (455 mg, 2.66 mmol), 9-benzyl-7-oxa-3,9-diazabicyclo[3.3.1]nonane (580 mg, 2.66 mmol) and cesium carbonate (2.77 g, 8.50 mmol) in DMA (10 ml) was stirred at 100° C. for 5 hours. The DMA was evaporated under reduce pressure, water (5 ml) was added and the mixture was extracted with methylene chloride. After evaporation, the crude product was purified by flash chromatography on silica gel eluting with 40 to 60% ethyl acetate in petroleum ether to afford 9-benzyl-7-(3-methoxy-4-nitrophenyl)-3-oxa-7,9-diazabicyclo[3.3.1]nonane (503 mg, 51%) as a yellow solid; NMR Spectrum: (CDCl₃) 2.96 (bs, 2H), 3.52-3.61 (m, 4H), 3.83-3.89 (m, 2H), 3.92 (s, 2H), 3.97 (s, 3H), 3.99-4.05 (m, 2H), 6.27 (d, 1H), 6.41 (dd, 1H), 7.29 (t, 1H), 7.35 (t, 2H), 7.40 (d, 2H), 8.08 (d, 1H).

A mixture of 9-benzyl-7-(3-methoxy-4-nitrophenyl)-3-oxa-7,9-diazabicyclo[3.3.1]nonane (500 mg, 1.35 mmol) and palladium 10% on charcoal (72 mg) in ethyl acetate (25 mL) and methanol (25 mL) was hydrogenated under 60 psi at 25° C. for 25 hours. The mixture was filtered and the filtrate was concentrated to dryness to afford the crude 4-(3-oxa-7,9-diazabicyclo[3.3.1]nonan-7-yl)-2-methoxyaniline (370 mg) which was used without further purification.

### Example 292

### 1-[3-[4-[(5-chloro-4-imidazo[1,2-a]pyridin-3-yl-pyrimidin-2-yl)amino]-3-methoxy-phenyl]-7-oxa-3,9-diazabicyclo[3.3.1]nonan-9-yl]ethanone

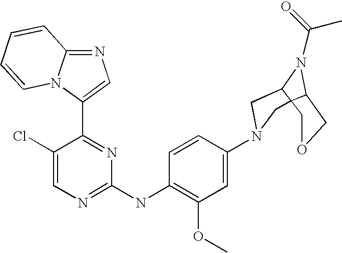

2-(3H-[1,2,3]triazolo[4,5-b]pyridin-3-yl)-1,1,3,3-tetramethylisouronium hexafluorophosphate(V) (76 mg, 0.20 mmol) was added to a stirred solution of N-(4-(3-oxa-7,9-diazabicyclo[3.3.1]nonan-7-yl)-2-methoxyphenyl)-5-chloro-4-(imidazo[1,2-a]pyridin-3-yl)pyrimidin-2-amine (Example 291, 60 mg, 0.13 mmol), N-ethyl-N-isopropylpropan-2-amine (0.066 mL, 0.38 mmol) and acetic acid (7.2 μL, 0.13 mmol) in methylene chloride (2 mL) at 25° C. This mixture was stirred for 18 hours and the solvent was removed under vacuum. The crude product was purified by flash chromatography on silica gel eluting with 1 to 4% methanolic ammonia in methylene chloride to afford 1-(7-(4-(5-chloro-4-(imidazo[1,2-a]pyridin-3-yl)pyrimidin-2-ylamino)-3-methoxyphenyl)-3-oxa-7,9-diazabicyclo[3.3.1]nonan-9-yl)ethanone (62 mg, 95%); NMR spectrum: 2.11 (s, 3H), 2.98-3.05 (m, 1H), 3.11-3.21 (m partially hidden by H2O, 1H), 3.75-3.83 (m, 1H), 3.78 (s, 3H), 3.85-3.94 (m, 2H), 3.95-4.03 (m, 2H), 4.12 (bs, 1H), 4.55 (bs, 1H), 6.49 (d, 1H), 6.63 (d, 1H), 6.89 (dd, 1H), 7.30 (d, 1H), 7.45 (ddd, 1H), 7.74 (d, 1H), 8.44 (s, 1H), 8.62 (s, 1H), 8.73 (s, 1H), 9.50 (bs, 1H); Mass spectrum: 520 (MH+).

### Example 293

### General Salt Screening Methodology

Solutions of the counter-ions are prepared in methanol to a concentration of 0.1M (for some counterions solubility limits the concentration to 0.05M).

Solutions of the test compound with concentrations of up to 0.1 M are prepared in methanol (solubility permitting. If 0.1M is not achievable a lower concentration is chosen). The salt screen is carried out in 96 well plates. Each counterion is screened in duplicate with a 1:1 molar ratio of counterion to drug. 100 μL of drug solution is titrated into 96 well plates in duplicate. The required amount of counterion is then also added. This volume will obviously depend on the concentration of the drug solution. This is repeated for all the counterions to be used in the screen.

Filled plates are covered then stored at 25° C. and at 50° C. until evaporation and crystallisation and has completed.

To determine if salt formation has been successful the plates are analysed using light microscopy. The presence of birefringent crystals under polarised light (a property of crystalline material) is an indication that a salt may have formed.

Using the general methodology 2-[4-[4-[(5-fluoro-4-imidazo[1,2-a]pyridin-3-yl-pyrimidin-2-yl)amino]-3-methoxy-phenyl]piperazin-1-yl]ethanol was screened against the following acids/counterions Acetic, Adipic, Benzenesulfonic, Benzoic, Citric, D,L-Lactic, D,L-mandelic, Ethanedisulfonic, Ethanesulfonic, Fumaric, Glutaric, Glycolic, Hippuric, HCl, Maleic, Malic, Malonic, Napadysilic, Phosphoric, Saccharin, Sulphuric. The results indicated that the following may have a propensity to form salts with the test compound: Acetic, Adipic, Benzenesulfonic, Citric, D,L-Lactic, D,L-mandelic, Ethanedisulfonic, Fumaric, Glutaric, Glycolic, Hippuric, HCl, Maleic, Malic, Malonic, Napadysilic, Phosphoric, Saccharin, Sulphuric.

### Example 293

### Preparation of Salts of 2-[4-[4-[(5-fluoro-4-imidazo[1,2-a]pyridin-3-yl-pyrimidin-2-yl)amino]-3-methoxy-phenyl]piperazin-1-yl]ethanol

The salts were made by dissolving 2-[4-[4-[(5-fluoro-4-imidazo[1,2-a]pyridin-3-yl-pyrimidin-2-yl)amino]-3-methoxy-phenyl]piperazin-1-yl]ethanol in 10 volumes of methanol heating to 55° C. and then adding, with stirring:—

### Phosphate Salt.

85% Phosphoric acid in water was added to the solution, 1 equivalent was added over 10 minutes with precipitation after 25% of acid added. The contents cooled to ambient (22° C.) and isolated.

### Chloride Salt.

36.5% hydrochloric acid added over 5 minutes, 1 eq added cooled to 0° C. and isolated.

### Sulphate Salt.

0.5 ml water added followed by sulphuric acid 1 eq over 10 minutes ppt formed towards the end of addition, cooled to 0° C. and isolated.

### Citrate Salt.

Citric acid 1 eq dissolved in 1.6 rel vol water and added to solution over 10 minutes cooled to 0° C. leading to oiling, allowed to warm to ambient and left to stir overnight. The solution warmed to 40° C. and cooled to 0° C. to isolate the solid.

### Besylate Salt

1 equivalent of benzene sulphonic acid in methanol and allowing the resultant mixture to crystallise by slow evaporation of the methanol.

All the solids were dried in a vacuum oven at 40° C. over night (16-18 hours).

### Tosylate Salt

Tosylate salt was made by dissolving a 1:1 stoichemetric mixture of 2-[4-[4-[(5-fluoro-4-imidazo[1,2-a]pyridin-3-yl-pyrimidin-2-yl)amino]-3-methoxy-phenyl]piperazin-1-yl]ethanol and toluene sulphonice acid in ethyl acetate.

### Fumarate Salt

Fumerate salt was prepared by mixing equimolar quantities of fumaric acid and 2-[4-[4-[(5-fluoro-4-imidazo[1,2-a]pyridin-3-yl-pyrimidin-2-yl)amino]-3-methoxy-phenyl]piperazin-1-yl]ethanol in methanol and leaving to evaporate.

### Glycolate Salt

Glycolate salt was produced by combination of 1:1 molar ratios of glycolic acid with 2-[4-[4-[(5-fluoro-4-imidazo[1,2-a]pyridin-3-yl-pyrimidin-2-yl)amino]-3-methoxy-phenyl]piperazin-1-yl]ethanol in excess methanol. This was then allowed to evaporate until solid was seen, upon this time solid was isolated by filtration.

### Edisylate Salt

Edisylate salt was produced by addition of 1:1 molar ratios of ethane sulphonic acid and 2-[4-[4-[(5-fluoro-4-imidazo[1,2-a]pyridin-3-yl-pyrimidin-2-yl)amino]-3-methoxy-phenyl]piperazin-1-yl]ethanol to acetone and allowed to evaporate until solid was produced. This was then filtered and analyzed.

### Example 294

### Preparation of 2-[4-[4-[(5-fluoro-4-imidazo[1,2-a]pyridin-3-yl-pyrimidin-2-yl)amino]-3-methoxy-phenyl]piperazin-1-yl]ethanol phosphate salt

A 2 l round bottom flask fitted equipped with an overhead stirrer, argon inerting line, pressure equalising dropping funnel and a temperature probe was purged with argon. 2-[4-[4-[(5-fluoro-4-imidazo[1,2-a]pyridin-3-yl-pyrimidin-2-yl)amino]-3-methoxy-phenyl]piperazin-1-yl]ethanol (155.8 g; 1.00 equiv; 336.13 mmoles) was charged to the flask. Initially 1090 ml methanol was added and the contents agitated. Then a further 310 ml methanol was added to give and the contents were heated to 58° C. to give an orangy/brown solution [Total Methanol 1400 mL; 34.59 moles; 1.40 L; 1.11 kg]. Phosphoric Acid (38.75 g; 1.00 equiv; 336.11 mmoles; 21.79 mL) was added over 30 minutes, from a pressure equalising dropping funnel, to the solution. A yellow solid was formed instantly on addition. The addition continued until all Phosphoric acid had been added. The reaction mixture was cooled to ambient temperature over 2 hours, and was stirred at ambient temperature overnight (16 hours), and then cooled further to 0° C. over 1 hour. The solid was filtered off onto a Whatman No 1 filter paper fitted to a glass sinter funnel. The filtration took 10 minutes. The solid was washed with methanol (300 mL; 7.41 moles; 300.00 mL; 237.51 g) which had been cooled to 0° C. in 2 equal portions (150 ml). The solid was discharged to a glass crystallising dish covered with a filter paper and dried to constant weight. After drying overnight net weight=169.92 g. After drying for a further 24 hours. (Total Time 38 hours), there was no change in weight. Weight=169.72 g (Theoretical yield=188.74 g)

### Methods

### Preparation of Starting Materials

### Method 1

### Preparation of 3-(2,5-dichloropyrimidin-4-yl)imidazo[1,2-a]pyridine

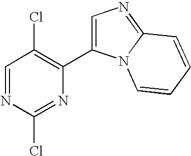

N-bromosuccinimide (12.9 g, 72.8 mmol) was added to a stirred solution of (E)-4-(2-butoxyvinyl)-2,5-dichloropyrimidine (18 g, 72.8 mmol) in dioxane (400 ml) and water (150 ml). The resulting solution was stirred for 1 hour at room temperature then 2-aminopyridine (6.86 g, 72.8 mmol) was added and the solution was heated to 65° C. for 4 h. Dioxane was evaporated and a saturated solution of sodium bicarbonate was added until pH=7. The to mixture was extracted with dichloromethane and the organic layer was washed with brine, dried on MgSO4, filtered and evaporated. Purification on silica gel, eluting with 5% MeOH in dichloromethane afforded 3-(2,5-dichloropyrimidin-4-yl)imidazo[1,2-a]pyridine in 54% yield; NMR spectrum: (CDCl₃) 7.15 (dd, 1H), 7.53 (ddd, 1H), 7.83 (d, 1H), 8.60 (s, 1H), 9.01 (s, 1H), 9.87 (d, 1H); Mass spectrum: 265 (MH+).

(E)-4-(2-butoxyvinyl)-2,5-dichloropyrimidine was obtained as follows:

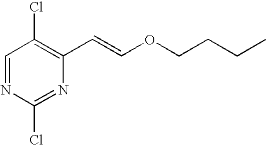

Triethylamine (19.4 ml, 139 mmol), 1-(vinyloxy)butane (18.0 ml, 139 mmol) and palladium(II) acetate (2.08 g, 9.30 mmol) were added to a stirred solution of 2,4,5-trichloropyrimidine (15 ml, 132 mmol) in polyethylene glycol 400 (150 ml). The solution was heated at 80° C. for 2 h. After cooling to 0° C., diethylether was added, the organic layer was separated and washed with brine, dried on MgSO4, filtered and evaporated to afford an orange oil. This oil was dissolved in 300 mL of a petroleum ether/AcOEt mixture (85:15), 40 g of silica was added and this suspension was filtered. The filtrate was evaporated to afford (E)-4-(2-butoxyvinyl)-2,5-dichloropyrimidine (18.0 g, 54%) as an orange oil; NMR spectrum: (CDCl₃) 0.97 (t, 3H), 1.41-1.50 (m, 2H), 1.70-1.78 (m, 2H), 4.03 (t, 2H), 6.09 (d, 1H), 8.07 (d, 1H), 8.33 (s, 1H).

### Method 2

### Preparation of 3-(2-chloro-5-fluoropyrimidin-4-yl)imidazo[1,2-a]pyridine

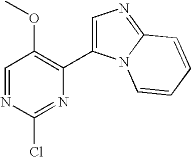

3-(2-chloro-5-fluoropyrimidin-4-yl)imidazo[1,2-a]pyridine was prepared according to Method 1 by reacting 2-aminopyridine with (E)-4-(2-butoxyvinyl)-2-chloro-5-fluoropyrimidine. (E)-4-(2-butoxyvinyl)-2-chloro-5-fluoropyrimidine was prepared using 2,4-dichloro-5-fluoropyrimidine as a starting material.

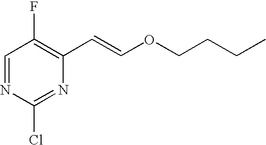

NMR spectrum: (CDCl₃) 0.97 (t, 3H), 1.41-1.50 (m, 2H), 1.69-1.78 (m, 2H), 4.01 (t, 2H), 5.91 (d, 1H), 8.00 (d, 1H), 8.21 (d, 1H).

3-(2-chloro-5-fluoropyrimidin-4-yl)imidazo[1,2-a]pyridine: NMR spectrum: (CDCl3) 7.18 (ddd, 1H), 7.54 (ddd, 1H), 7.85 (d, 1H), 8.46 (d, 1H), 8.61 (d, 1H), 9.99 (d, 1H); Mass spectrum: 249 (MH+).

### Method 3

### Preparation of 3-(2-chloro-5-methoxypyrimidin-4-yl)imidazo[1,2-a]pyridine

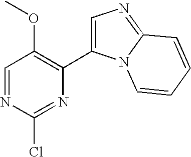

N-bromosuccinimide (4.15 g, 23.3 mmol) was added to a stirred solution of (E)-2-chloro-4-(2-ethoxyvinyl)-5-methoxypyrimidine (5 g, 23.2 mmol) dissolved in dioxane (100 mL) and water (40 mL) at 25° C. The resulting solution was stirred for 1 hour then 2-aminopyridine (2.19 g, 23.2 mmol) was added and the resulting solution was stirred at 85° C. for 2 hours. The reaction mixture was diluted with EtOAc and neutralised with a saturated solution of sodium bicarbonate. The mixture was extracted with EtOAc and the organic layer was washed with brine and dried over MgSO4. After evaporation the crude product was purified by flash chromatography on silica gel eluting with 0 to 4% methanol in dichloromethane. Trituration with diethyl ether afforded 3-(2-chloro-5-methoxypyrimidin-4-yl)imidazo[1,2-a]pyridine (2.60 g, 42%) as a pale beige solid; NMR spectrum: 4.13 (s, 3H), 7.31 (dd, 1H), 7.61 (dd, 1H), 7.85 (d, 1H), 8.57 (s, 1H), 8.71 (s, 1H), 9.89 (d, 1H); Mass spectrum: 261 (MH+).

(E)-2-chloro-4-(2-ethoxyvinyl)-5-methoxypyrimidine was obtained as follows:

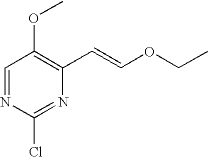

Borane tetrahydrofuran complex (2.8 mL, 2.8 mmol) was added dropwise at 0° C. and under nitrogen to a stirred solution of ethoxyethyne (˜40% wt solution in hexanes, 2.0 mL, 8.4 mmol) dissolved in THF (10 mL). The resulting solution was stirred at 25° C. for 4 hours. The mixture was thoroughly degassed with nitrogen then 2,4-dichloro-5-methoxypyrimidine (500 mg, 2.79 mmol), bis(triphenylphosphine) palladium(II) chloride (137 mg, 0.20 mmol), potassium phosphate (1.18 g, 5.59 mmol) and water (0.30 mL, 16.7 mmol) were added. The resulting solution was stirred at 60° C. for 18 hours. THF was evaporated, the residue was taken-up in water and the mixture was extracted with EtOAc. The combined organic layers were washed with brine, dried over MgSO4 and concentrated to dryness. The crude product was purified by flash chromatography on silica gel eluting with 0 to 10% ethyl acetate in dichloromethane. The solvent was evaporated to afford (E)-2-chloro-4-(2-ethoxyvinyl)-5-methoxypyrimidine (430 mg, 71%) as an orange oil which crystallised on standing; NMR spectrum: 1.29 (t, 3H), 3.91 (s, 3H), 4.08 (q, 2H), 5.96 (d, 1H), 7.90 (d, 1H), 8.29 (s, 1H).

### Method 4

### Preparation of 3-(2-chloro-5-bromopyrimidin-4-yl)imidazo[1,2-a]pyridine

This compound was prepared from 2,4-dichloro-5-bromopyrimidine according to Method 3.

### 5-bromo-2-chloro-4-[(E)-2-ethoxyethenyl]pyrimidine

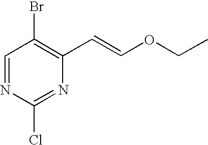

NMR spectrum: (CDCl₃) 1.40 (t, 3H), 4.10 (q, 2H), 6.12 (d, 1H), 8.06 (d, 1H), 8.44 (s, 1H).

### 3-(2-chloro-5-bromopyrimidin-4-yl)imidazo[1,2-a]pyridine

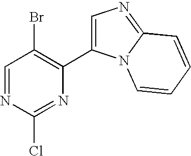

NMR spectrum: 7.29 (dd, 1H), 7.62 (dd, 1H), 7.86 (d, 1H), 8.90 (s, 1H), 9.03 (s, 1H), 9.48 (d, 1H); Mass spectrum: 309 (MH+).

### Method 5

### Preparation of 3-(2-chloro-5-methylpyrimidin-4-yl)imidazo[1,2-a]pyridine

This compound was prepared from 2,4-dichloro-5-methylpyrimidine according to Method 3.

### 5-methyl-2-chloro-4-[(E)-2-ethoxyethenyl]pyrimidine

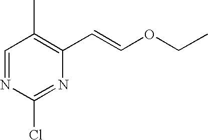

NMR spectrum: 1.29 (t, 3H), 2.16 (t, 3H), 4.11 (q, 2H), 5.92 (d, 1H), 7.89 (d, 1H), 8.33 (s, 1H).

### 3-(2-chloro-5-methylpyrimidin-4-yl)imidazo[1,2-a]pyridine

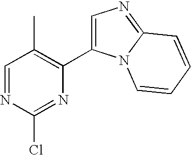

NMR spectrum: 2.52 (s, 3H), 7.24 (dd, 1H), 7.57 (ddd, 1H), 7.83 (d, 1H), 8.45 (s, 1H), 8.67 (s, 1H), 9.60 (d, 1H); Mass spectrum: 245 (MH+).

### Method 6

### Preparation of 3-(2-chloro-5-trifluoromethylpyrimidin-4-yl)imidazo[1,2-a]pyridine

This compound was prepared from 2,4-dichloro-5-trifluoromethylpyrimidine according to Method 3.

### 2-chloro-4-[(E)-2-ethoxyethenyl]-5-(trifluoromethyl)pyrimidine

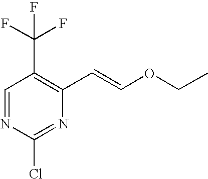

NMR spectrum: (CDCl₃) 1.41 (t, 3H), 4.12 (q, 2H), 5.98 (dq, 1H), 8.21 (d, 1H), 8.57 (s, 1H).

### 3-[2-chloro-5-(trifluoromethyl)pyrimidin-4-yl]imidazo[1,2-a]pyridine

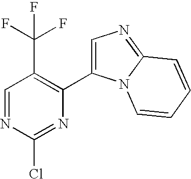

NMR spectrum: 7.16 (dd, 1H), 7.55 (dd, 1H), 7.86 (d, 1H), 8.51 (s, 1H), 8.89 (s, 1H), 9.67 (d, 1H); Mass spectrum: 229 (MH+).

### Method 7

### Preparation of 2-methoxy-4-morpholin-4-ylaniline

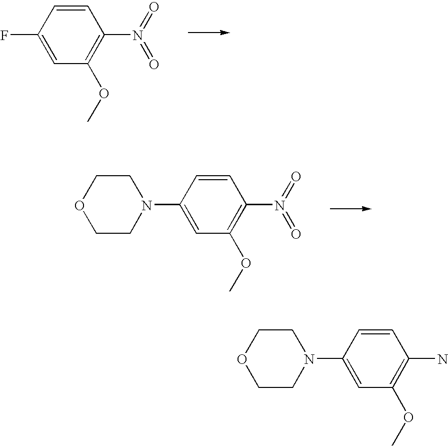

N,N-diisopropylethylamine (2.45 ml, 14.0 mmol) and morpholine (1.12 ml, 12.9 mmol) were added to a stirred solution of 4-fluoro-2-methoxy-1-nitrobenzene (2.0 g, 11.7 mmol) in DMA (10 ml). The resulting solution was stirred at 90° C. overnight under argon. The mixture was allowed to cool to room temperature and water was added. The yellow precipitate was filtered, washed with water and dried under vacuum at 50° C. over P2O5 to afford 4-(3-methoxy-4-nitrophenyl)morpholine (2.63 g, 94% yield); NMR spectrum: 3.38-3.44 (m, 4H), 3.70-3.76 (m, 4H), 3.91 (s, 3H), 6.55 (d, 1H), 6.60 (dd, 1H), 7.70 (d, 1H).

A suspension of 4-(3-methoxy-4-nitrophenyl)morpholine (2.6 g, 10.9 mmol) and platinum(IV) oxide (0.248 g, 1.09 mmol) in EtOH (200 ml) was hydrogenated under 1.3 bars at 25° C. for 90 minutes. The resulting suspension was filtered and the filtrate was concentrated to dryness to afford 2-methoxy-4-morpholinoaniline (2.26 g, 99%) as a dark purple oil which solidified on standing; NMR spectrum: 2.89-2.95 (m, 4H), 3.67-3.76 (m, 4H), 3.74 (s, 3H), 4.22 (bs, 2H), 6.29 (dd, 1H), 6.50 (d, 1H), 6.53 (d, 1H).

The following anilines were prepared in a similar manner using the indicated amine as the starting material:

| Aniline | Starting amine | NMR Spectrum |
|---|---|---|
| 1-(4-(4-amino-3- | N-acetylpiperazine | 2.02 (s, 3H), 2.84-2.90 (m, 2H), |
| methoxyphenyl)piperazin- | | 2.90-2.96 (m, 2H), 3.50-3.58 (m, |
| 1-yl)ethanone | | 4H), 3.74 (s, 3H), 4.27 (bs, 2H), |
| | | 6.31 (dd, 1H), 7.52 (d, 1H), 7.53 (d, |
| | | 1H) |
| 2-methoxy-4-pyrrolidin-1- | pyrrolidine | 1.85-1.96 (m, 4H), 3.06-3.17 (m, |
| ylaniline | | 4H), 3.74 (s, 3H), 3.91 (bs, 2H), |
| | | 5.95 (dd, 1H), 6.14 (d, 1H), 6.51 (d, |
| | | 1H) |
| 2-[4-(4-amino-3- | 2-(piperazin-1-yl)ethanol | 2.42 (t, 2H), 2.50-2.56 (m, 4H), |
| methoxyphenyl)piperazin- | | 2.89-2.97 (m, 4H), 3.48-3.55 (m, |
| 1-yl]ethanol | | 2H), 3.73 (s, 3H), 4.20 (bs, 2H), |
| | | 4.40 (t, 1H), 6.27 (dd, 1H), 6.48 (d, |
| | | 1H), 6.51 (d, 1H) |
| 2-methoxy-4-[4-(2- | methoxyethylpiperazine | 2.50 (t partially hidden by |
| methoxyethyl)piperazin-1- | | DMSOd5. 2H), 2.51-2.55 (m, 4H), |
| yl]aniline | | 2.90-2.95 (m, 4H), 3.24 (s, 3H), |
| | | 3.45 (t, 2H), 3.73 (s, 3H), 4.19 (bs, |
| | | 2H), 6.27 (dd, 1H), 6.48 (d, 1H), |
| | | 6.51 (d, 1H) |
| (3S)-1-(4-amino-3- | (S)-3-hydroxypyrrolidine | 1.78-1.87 (m, 1H), 1.97-2.06 (m, |
| methoxyphenyl)pyrrolidin- | | 1H), 2.92-2.99 (m, 1H), |
| 3-ol | | 3.10-3.17 (m, 1H), 3.18-3.26 (m, 1H), |
| | | 3.31-3.37 (m partially hidden by H2O, |
| | | 1H), 3.74 (s, 3H), 3.90 (bs, 2H), |
| | | 4.32-4.39 (m, 1H), 4.86 (d, 1H), |
| | | 5.91 (dd, 1H), 6.10 (d, 1H), 6.52 (d, |
| | | 1H) |
| (3R)-1-(4-amino-3- | (R)-3-hydroxypyrrolidine | 1.78-1.87 (m, 1H), 1.97-2.06 (m, |
| methoxyphenyl)pyrrolidin- | | 1H), 2.92-2.99 (m, 1H), |
| 3-ol | | 3.10-3.17 (m, 1H), 3.18-3.26 (m, 1H), |
| | | 3.31-3.37 (m partially hidden by H2O, |
| | | 1H), 3.74 (s, 3H), 3.90 (bs, 2H), |
| | | 4.32-4.39 (m, 1H), 4.86 (d, 1H), |
| | | 5.91 (dd, 1H), 6.10 (d, 1H), 6.51 (d, |
| | | 1H) |
| 2-methoxy-4-(1,4- | 1,4-oxazepane | 1.84-1.92 (m, 2H), 3.41-3.47 (m, |
| oxazepan-4-yl)aniline | | 4H), 3.53-3.59 (m, 2H), |
| | | 3.67-3.71 (m, 2H), 3.73 (s, 3H), 3.97 (bs, 2H), |
| | | 6.12 (dd, 1H), 6.29 (d, 1H), 6.50 (d, |
| | | 1H) |
| 2-[4-(4-amino-3- | 1-(morpholinocarbonyl- | 2.50-2.56 (m, 4H), 2.90-2.97 (m, |
| methoxyphenyl)piperazin- | methyl)piperazine | 4H), 3.19 (s, 2H), 3.41-3.46 (m, |
| 1-yl]-1-morpholin-4- | | 2H), 2.52-2.56 (m, 2H), 3.57 (bs, |
| ylethanone | | 4H), 3.74 (s, 3H), 4.21 (bs, 2H), |
| | | 6.28 (dd, 1H), 6.48 (d, 1H), 6.51 (d, |
| | | 1H) |
| 4-(3,5-dimethylpiperazin- | 2,6-dimethylpiperazine | 0.99 (d, 6H), 1.94-2.03 (m, 2H), |
| 1-yl)-2-methoxyaniline | | 2.08 (bs, 1H), 2.79-2.88 (m, 2H), |
| | | 3.20-3.27 (m, 2H), 3.73 (s, 3H), |
| | | 4.17 (bs, 2H), 6.26 (dd, 1H), |
| | | 6.46 (d, 1H), 6.50 (d, 1H) |
| 4-(4-amino-3- | N,N-dimethylpiperazine- | 2.80 (s, 6H), 2.96-3.01 (m, 4H), |
| methoxyphenyl)-N,N- | 1-sulfonamide | 3.24-3.29 (m, 4H), 3.74 (s, 3H), |
| dimethylpiperazine-1- | | 4.28 (bs, 2H), 6.32 (d, 1H), 6.52 (d, |
| sulfonamide | | 1H), 6.53 (d, 1H) |
| (3R)-1-(4-amino-3- | (3R)—N,N- | 1.70-1.82 (m, 1H), 2.05-2.13 (m, |
| methoxyphenyl)-N,N- | dimethylpyrrolidin-3- | 1H), 2.18 (s, 6H), 2.70-2.79 (m, |
| dimethylpyrrolidin-3- | amine | 1H), 2.90-2.98 (m, 1H), |
| amine | | 3.11-3.18 (m, 1H), 3.18-3.25 (m, 1H), |
| | | 2.28-2.35 (m partially hidden by H2O, |
| | | 1H), 3.74 (s, 3H), 3.92 (bs, 2H), |
| | | 5.94 (dd, 1H), 6.13 (d, 1H), 6.52 (d, |
| | | 1H) |
| 4-(1,1-dioxo-1,4- | 1,1-dioxo-thiomorpholine | 3.11-3.18 (m, 4H), 3.46-3.53 (m, |
| thiazinan-4-yl)-2- | | 4H), 3.75 (s, 3H), 4.32 (bs, 2H), |
| methoxyaniline | | 6.38 (dd, 1H), 6.54 (d, 1H), 6.57 (d, |
| | | 1H) |
| (3S)-1-(4-amino-3- | (3S)—N,N- | 1.70-1.82 (m, 1H), 2.05-2.13 (m, |
| methoxyphenyl)-N,N- | dimethylpyrrolidin-3- | 1H), 2.18 (s, 6H), 2.70-2.79 (m, |
| dimethylpyrrolidin-3- | amine | 1H), 2.90-2.98 (m, 1H), |
| amine | | 3.11-3.18 (m, 1H), 3.18-3.25 (m, 1H), |
| | | 2.28-2.35 (m partially hidden by H2O, |
| | | 1H), 3.74 (s, 3H), 3.92 (bs, 2H), |
| | | 5.94 (dd, 1H), 6.13 (d, 1H), 6.52 (d, |
| | | 1H) |
| 2-methoxy-4-(4- | 1- | 2.91 (s, 3H), 3.00-3.06 (m, 4H), |
| methylsulfonylpiperazin- | methylsulfonylpiperazine | 3.19-3.26 (m, 4H), 3.75 (s, 3H), |
| 1-yl)aniline | | 4.28 (bs, 2H), 6.33 (dd, 1H), |
| | | 6.53 (d, 1H), 6.54 (d, 1H) |
| N-[(3R)-1-(4-amino-3- | N-pyrrolidin-3- | 1.76-1.85 (m, 1H), 1.80 (s, 3H), |
| methoxyphenyl)pyrrolidin- | ylacetamide | 2.10-2.19 (m, 1H), 2.94 (dd, 1H), |
| 3-yl]acetamide | | 3.09-3.16 (m, 1H), 3.23-3.29 (m, |
| | | 1H), 3.36 (dd, 1H), 3.74 (s, 3H), |
| | | 3.95 (bs, 2H), 4.28-4.36 (m, 1H), |
| | | 5.94 (dd, 1H), 6.13 (d, 1H), 6.52 (d, |
| | | 1H), 8.11 (d, 1H) |
| 1-[4-(4-amino-3- | 1-(1,4-diazepan-1- | 1.74-1.81 (m, 1H), 1.82-1.90 (m, |
| methoxyphenyl)-1,4- | yl)ethanone | 1H), 1.98 (s, 3H), 3.27-3.35 (m |
| diazepan-1-yl]ethanone | | partially hidden by H2O, 2H), |
| | | 3.35-3.44 (m, 3H), 3.50-3.60 (m, 3H), |
| | | 3.73 (s, 3H), 3.99 (bs, 2H), |
| | | 6.10-6.15 (m, 1H), 6.28 (d, 1H), |
| | | 6.47-6.53 (m, 1H) |
| 1-[4-(4-amino-3- | 2,6-dimethylpiperazine | 1.31 (bs, 6H), 2.04 (s, 3H), |
| methoxyphenyl)-2,6- | (see note a) | 2.61 (bs, 2H), 3.20 (d, 2H), 3.76 (s, 3H), |
| dimethylpiperazin-1- | | 4.04 (bs, 1H), 4.29 (bs, 2H), |
| yl]ethanone | | 4.45 (bs, 2H), 6.33 (dd, 1H), 6.48 (d, |
| | | 1H), 6.55 (d, 1H) |
| 1-[trans-4-(4-amino-3- | trans-2,5- | 0.73 (d, 1.5H), 0.83 (d, 1.5H), |
| methoxyphenyl)-2,5- | dimethylpiperazine | 1.15 (d, 1.5H), 1.27 (d, 1.5H), 1.97 (s, |
| dimethylpiperazin-1- | (see note b) | 1.5H), 2.08 (s, 1.5H), 2.82-2.92 (m, |
| yl]ethanone | | 1H), 2.95-3.04 (m, 0.5H), |
| | | 3.07-3.16 (m, 1H), 3.46-3.55 (m, 0.5H), |
| | | 3.57-3.66 (m, 0.5H), 3.74 (s, 3H), |
| | | 3.83 (bs, 1H), 4.08-4.21 (m, 1H), 4.18 (s, |
| | | 2H), 4.69 (bs, 0.5H), 6.23 (d, 1H), |
| | | 6.46 (d, 1H), 6.52 (d, 1H) |
| 4-(4-amino-3-methoxy- | piperazin-2-one | 3.15-3.22 (m, 2H), 3.22-3.29 (m, |
| phenyl)piperazin-2-one | | 2H), 3.50 (s, 2H), 3.75 (s, 3H), |
| | | 4.26 (bs, 2H), 6.30 (dd, 1H), 6.53 (d, |
| | | 1H), 6.54 (d, 1H), 7.91 (bs, 1H) |

Notes:
(a) the intermediate 1-[4-(3-methoxy-4-nitrophenyl)-2,6-dimethylpiperazin-1-yl]ethanone was obtained as follows: acetic anhydride (6.46 ml, 58 mmol) was added to a stirred solution of 1-(3-methoxy-4-nitrophenyl)-3,5-dimethylpiperazine (770 mg, 2.90 mmol) dissolved in dichloromethane (10 ml) at 25° C. The resulting solution was stirred at 25° C. for 1 hour. After evaporation, the crude product was purified by flash chromatography on silica gel with 0 to 5% MeOH/EtOAc to afford 1-(4-(3-methoxy-4-nitrophenyl)-2,6-dimethylpiperazin-1-yl)ethanone (840 mg, 94%) as a yellow solid.
(b) the intermediate 1-[trans-4-(3-methoxy-4-nitrophenyl)-2,5-dimethylpiperazin-1-yl]ethanone was obtained as follows: acetic anhydride (5.93 ml, 53.3 mmol) was added to a stirred solution of trans-1-(3-methoxy-4-nitrophenyl)-2,5-dimethylpiperazine (707 mg, 2.66 mmol) dissolved in dichloromethane (10 ml) at 25° C. The resulting solution was stirred at 25° C. for 1 hour. After evaporation, the crude product was purified by flash chromatography on silica gel with 0 to 5% MeOH/EtOAc to afford 1-(trans-4-(3-methoxy-4-nitrophenyl)-2,5-dimethylpiperazin-1-yl)ethanone (830 mg, 100%) as a yellow solid.

### Method 8

### Preparation of 2-methoxy-5-morpholin-4-ylaniline

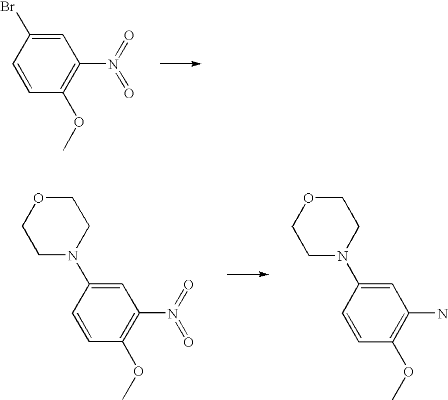

4-bromo-1-methoxy-2-nitrobenzene (1.19 g, 5.13 mmol), morpholine (0.447 ml, 5.13 mmol), cesium carbonate (3.34 g, 10.2 mmol), palladium (II) acetate (0.092 g, 0.41 mmol) and 9,9-dimethyl-4,5-bis(diphenylphosphino)xanthene (0.356 g, 0.62 mmol) in 1,4-dioxane (10 ml) were stirred at 90° C. for 5 hours. After dilution with dichloromethane and filtration, the reaction mixture was washed with water and the organic layer dried (MgSO4), filtered and evaporated. The crude product was purified by flash chromatography on silica gel eluting with 0 to 100% EtOAc/petroleum ether to afford 4-(4-methoxy-3-nitrophenyl)morpholine (0.652 g, 53%) as a solid; NMR spectrum: (CDCl₃) 3.08-3.14 (m, 4H), 3.84-3.89 (m, 4H), 3.92 (s, 3H), 7.03 (d, 1H), 7.12 (dd, 1H), 7.32 (d, 1H).

A mixture of 4-(4-methoxy-3-nitrophenyl)morpholine (1.54 g, 6.46 mmol) and platinum (IV) oxide in ethanol (40 mL) and ethyl acetate (40 mL), was hydrogenated under 1200 mbar at 25° C. for 6 hours. The reaction mixture was filtered and the filtrate was concentrated to dryness to afford the crude 2-methoxy-5-morpholinoaniline (97%) as a solid; NMR spectrum: 2.88-2.93 (m, 4H), 3.67 (s, 3H), 3.67-3.72 (m, 4H), 4.59 (bs, 2H), 6.09 (dd, 1H), 6.30 (d, 1H), 6.65 (d, 1H).

The following anilines have been prepared in a similar manner using the indicated amine as a starting material:

| Aniline | Starting amine | NMR Spectrum |
|---|---|---|
| (3S)-1-(3-amino-4- | (3S)-pyrrolidin-3-ol | 1.78-1.86 (m, 1H), 1.96-2.05 (m, |
| methoxyphenyl)pyrrolidin- | | 1H), 2.93 (dd, 1H), 3.07-3.15 (m, |
| 3-ol | | 1H), 3.16-3.24 (m, 1H), |
| | | 3.27-3.33 (m partially hidden by H2O, 1H), |
| | | 3.63 (s, 3H), 4.31-4.37 (m, 1H), |
| | | 4.52 (bs, 2H), 4.86 (d, 1H), |
| | | 5.68 (dd, 1H), 5.92 (d, 1H), 6.62 (d, 1H) |
| 2-methoxy-5-pyrrolidin-1- | Pyrrolidine | 1.85-1.95 (m, 4H), 3.04-3.13 (m, |
| ylaniline | | 4H), 3.63 (s, 3H), 4.52 (bs, 2H), |
| | | 5.72 (dd, 1H), 5.95 (d, 1H), 6.62 (d, |
| | | 1H) |
| 1-[4-(3-amino-4- | N-acetylpiperazine | 2.02 (s, 3H), 2.84-2.90 (m, 2H), |
| methoxyphenyl)piperazin- | | 2.91-2.97 (m, 2H), 3.50-3.58 (m, |
| 1-yl]ethanone | | 4H), 3.68 (s, 3H), 4.60 (bs, 2H), |
| | | 6.11 (dd, 1H) < 6.32 (d, 1H), 6.66 (d, |
| | | 1H) |
| (3R)-1-(3-amino-4- | (3R)—N,N- | 1.70-1.90 (m, 1H), 2.05-2.13 (m, |
| methoxyphenyl)-N,N- | dimethylpyrrolidin-3- | 1H), 2.17 (s, 6H), 2.69-2.78 (m, |
| dimethylpyrrolidin-3- | amine | 1H), 2.97-2.95 (m, 1H), |
| amine | | 3.08-3.16 (m, 1H), 3.16-3.23 (m, 1H), |
| | | 3.25-3.31 (m, 1H), 3.63 (s, 3H), 4.52 (bs, |
| | | 2H), 5.71 (dd, 1H), 5.94 (d, 1H), |
| | | 6.62 (d, 1H) |
| (3S)-1-(3-amino-4- | (3S)—N,N- | 1.70-1.90 (m, 1H), 2.05-2.13 (m, |
| methoxyphenyl)-N,N- | dimethylpyrrolidin-3- | 1H), 2.17 (s, 6H), 2.69-2.78 (m, |
| dimethylpyrrolidin-3- | amine | 1H), 2.97-2.95 (m, 1H), |
| amine | | 3.08-3.16 (m, 1H), 3.16-3.23 (m, 1H), |
| | | 3.25-3.31 (m, 1H), 3.63 (s, 3H), 4.52 (bs, |
| | | 2H), 5.71 (dd, 1H), 5.94 (d, 1H), |
| | | 6.62 (d, 1H) |
| (3R)-1-(3-amino-4- | (3R)-pyrrolidin-3-ol | 1.78-1.86 (m, 1H), 1.96-2.05 (m, |
| methoxyphenyl)pyrrolidin- | | 1H), 2.93 (dd, 1H), 3.07-3.15 (m, |
| 3-ol | | 1H), 3.16-3.24 (m, 1H), |
| | | 3.27-3.33 (m partially hidden by H2O, 1H), |
| | | 3.63 (s, 3H), 4.31-4.37 (m, 1H), |
| | | 4.52 (bs, 2H), 4.86 (d, 1H), |
| | | 5.68 (dd, 1H), 5.92 (d, 1H), 6.62 (d, 1H) |
| 2-methoxy-5-(4- | 4-methoxypiperidine | (CDCl3) 1.66-1.76 (m, 2H), |
| methoxypiperidin-1- | | 1.97-2.05 (m, 2H), 2.73-2.82 (m, 2H), |
| yl)aniline | | 3.27-3.33 (m, 1H), 3.32-3.37 (m |
| | | partially hidden by H2O, 2H), |
| | | 3.74 (bs, 2H), 3.80 (s, 3H), 6.32 (dd, |
| | | 1H), 6.41 (d, 1H), 6.69 (d, 1H) |

### Method 9

### Preparation of [1-(4-amino-3-methoxyphenyl)pyrrolidin-2-yl]-morpholin-4-ylmethanone

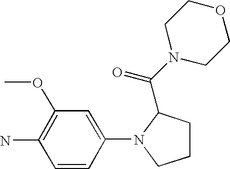

4-fluoro-2-methoxy-1-nitrobenzene (0.5 g, 2.9 mmol) and DL-proline (0.67 g, 5.8 mmol) in DMSO (2 mL) were stirred at 110° C. for 3 hours. The reaction mixture was cooled and was diluted with water to give a precipitate which was collected by filtration and dried under vacuum to give 1-(3-methoxy-4-nitrophenyl)pyrrolidine-2-carboxylic acid (0.57 g, 73%) as a brown solid; NMR spectrum: 1.94-2.09 (m, 2H), 2.09-2.17 (m, 1H), 2.24-2.34 (m, 1H), 3.42-3.50 (m, 1H), 3.52-3.61 (m, 1H), 3.88 (s, 3H), 4.41-4.48 (m, 1H), 6.14 (s, 1H), 6.16 (d, 1H), 7.82 (d, 1H).

Oxalyl chloride (0.572 mL, 6.76 mmol) was added to a mixture of 1-(3-methoxy-4-nitrophenyl)pyrrolidine-2-carboxylic acid (360 mg, 1.35 mmol) and triethylamine (0.37 mL, 2.7 mmol) in dichloromethane (3 mL) at 25° C. The reaction mixture was stirred at 25° C. for 1 hour then quenched with a saturated aqueous solution of ammonium chloride and extracted with dichloromethane. The organic phase was dried over magnesium sulfate and concentrated. The crude product was purified by flash chromatography on silica gel eluting with 0 to 50% ethyl acetate in dichloromethane to afford [1-(3-methoxy-4-nitro-phenyl)pyrrolidin-2-yl]-morpholino-methanone (210 mg, 46%) as a yellow foam; NMR spectrum: 1.89-2.06 (m, 3H), 2.22-2.34 (m, 1H), 3.46-3.71 (m, 9H), 3.72-3.81 (m, 1H), 3.88 (s, 3H), 4.94-5.02 (m, 1H), 6.00 (bs, 1H), 6.09 (bs, 1H), 7.90 (d, 1H).

A suspension of [1-(3-methoxy-4-nitro-phenyl)pyrrolidin-2-yl]-morpholino-methanone (320 mg, 0.95 mmol) and platinum (IV) oxide (108 mg, 0.48 mmol) in ethyl acetate (20 mL)/ethanol (20 mL) was hydrogenated under 55 psi at 25° C. for 2 hours. The reaction mixture was filtered and the filtrate was concentrated to dryness to afford the crude product. Purification by flash chromatography on silica gel eluting with 0 to 10% methanol in dichloromethane afforded [1-(4-amino-3-methoxy-phenyl)pyrrolidin-2-yl]-morpholino-methanone (266 mg, 91%) as a brown foam; NMR spectrum: 1.80-1.87 (m, 1H), 1.89-1.98 (m, 2H), 2.16-2.26 (m, 1H), 3.21-3.28 (m, 1H), 3.34 (bs, 1H), 3.35-3.41 (m, 1H), 3.43-3.64 (m, 6H), 3.71 (s, 3H), 3.71-3.77 (m, 1H), 3.91 (bs, 2H), 4.59 (dd, 1H), 5.81 (dd, 1H), 5.98 (d, 1H), 6.49 (d, 1H).

### Method 10

### Preparation of 2-methoxy-4-[2-[4-(2-methoxyethyl)piperazin-1-yl]ethoxy]aniline

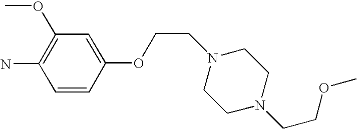

Tetrabutylammonium bromide (188 mg, 0.58 mmol) and a 5N aqueous solution of potassium hydroxide (2.16 mL, 10.8 mmol) were added to a stirred solution of 4-fluoro-2-methoxy-1-nitrobenzene (500 mg, 2.92 mmol) and tert-butyl 4-(2-hydroxyethyl)piperazine-1-carboxylate (673 mg, 2.92 mmol) dissolved in toluene (2.5 mL). The resulting mixture was stirred at 60° C. for 15 hours. Purification of the crude product by flash chromatography on silica gel eluting with 10 to 100% ethyl acetate in petroleum ether afforded tert-butyl 4-(2-(3-methoxy-4-nitrophenoxy)ethyl)piperazine-1-carboxylate (560 mg, 50.2%) as a pale yellow gum; NMR spectrum: 1.39 (s, 9H), 2.40-2.48 (m, 4H), 2.74 (t, 2H), 3.27-3.34 (m partially hidden by H2O, 4H), 3.93 (s, 3H), 4.23 (t, 2H), 6.68 (dd, 1H), 6.81 (d, 1H), 7.95 (d, 1H).

This product (551 mg, 1.44 mmol) was dissolved in a 4M solution of hydrogen chloride in dioxane (4.50 mL) and the solution was stirred overnight at 25° C. The reaction mixture was filtered, the solid was taken up in dichloromethane and basified with NH3/MeOH 7N. The resulting suspension was filtered and the filtrate was concentrated to dryness to afford crude 1-(2-(3-methoxy-4-nitrophenoxy)ethyl)piperazine as a yellow gum. This product was dissolved in DMA (7 mL) then potassium carbonate (399 mg, 2.89 mmol) and 2-bromoethyl methyl ether (163 μl, 1.73 mmol) were added. The resulting suspension was stirred at 50° C. for 20 hours. The reaction mixture was filtered and the filtrate was evaporated and purified by preparative HPLC using a Waters SunFire reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent. The fractions containing the desired compound were evaporated to dryness to afford 1-(2-(3-methoxy-4-nitrophenoxy)ethyl)-4-(2-methoxyethyl)piperazine (300 mg, 61.2%); NMR spectrum: 2.41 (bs, 8H), 2.44 (t, 2H), 2.48 (bs, 4H), 2.70 (t, 2H), 3.41 (t, 2H), 3.93 (s, 3H), 4.21 (t, 2H), 6.02 (bs, 1H), 6.67 (dd, 1H), 6.80 (d, 1H), 7.94 (d, 1H).

1-(2-(3-methoxy-4-nitrophenoxy)ethyl)-4-(2-methoxyethyl)piperazine was hydrogenated in the presence of platinum (IV) oxide following the procedure described in Method 10 to afford the title compound; NMR spectrum: 2.42 (bs, 8H), 3.43 (t, 2H), 2.60 (t, 2H), 3.22 (s, 3H), 3.40 (t, 2H), 3.73 (s, 3H), 3.93 (t, 2H), 4.22 (bs, 2H), 6.28 (dd, 1H), 6.45 (d, 1H), 6.52 (d, 1H).

### Method 11

### Preparation of (3S)-1-(3-amino-4-methylphenyl)-N,N-dimethylpyrrolidin-3-amine

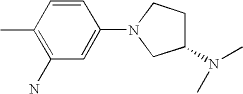

The title compound was prepared starting from 4-bromo-2-nitrotoluene and (3S)-3-(dimethylamino)pyrrolidine using the procedure described in Method 8. NMR spectrum: 1.69-1.82 (m, 1H), 1.93 (s, 3H), 2.06-2.19 (m, 1H), 2.18 (6H), 2.69-2.78 (m, 1H), 2.89-2.95 (m, 1H), 3.09-3.17 (m, 1H), 3.19-3.25 (m, 1H), 3.28-3.34 (m partially hidden by H2O, 1H), 4.54 (bs, 2H), 5.73 (dd, 1H), 5.86 (d, 1H), 6.68 (d, 1H).

### Method 12

### Preparation of 1-[4-(4-amino-3-methoxyphenyl)piperidin-1-yl]ethanone

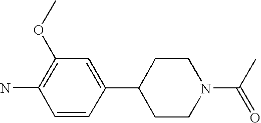

A mixture of tert-butyl 4-(trifluoromethylsulfonyloxy)-5,6-dihydropyridine-1(2H)-carboxylate (Journal of Medicinal Chemistry, 2006, vol. 49, p. 7450, 3.8 g, 11.4 mmol), tert-butyl (2-methoxy-4-(4,4,5,5-tetramethyl-1,3,2-dioxaborolan-2-yl)phenyl)carbamate (US2005/020619, 4.41 g, 12.6 mmol), a saturated aqueous solution of sodium bicarbonate (20 mL) and tetrakis (triphenylphosphine) palladium (0.265 g, 0.23 mmol) in dimethoxyethane (40 mL) was stirred at 90° C. overnight. The reaction mixture was diluted with water and extracted with EtOAc. The combined organic layers were washed with brine, dried over MgSO4 and concentrated. The crude product was purified by flash chromatography on silica gel eluting with 0 to 20% ethyl acetate in petroleum ether to afford tert-butyl 4-(4-(tert-butoxycarbonylamino)-3-methoxyphenyl)-5,6-dihydropyridine-1(2H)-carboxylate (3.70 g, 80%) as a pale yellow foam; NMR spectrum: 1.43 (s, 9H), 1.45 (s, 9H), 2.42-2.48 (m, 2H), 3.50-3.57 (m, 2H), 3.83 (s, 3H), 3.99 (bs, 2H), 6.12 (bs, 1H), 6.96 (dd, 1H), 7.03 (d, 1H), 7.65 (d, 1H), 7.87 (s, 1H).

A suspension of tert-butyl 4-(4-(tert-butoxycarbonylamino)-3-methoxyphenyl)-5,6-dihydropyridine-1(2H)-carboxylate (3.7 g, 9.15 mmol) and 10% palladium on charcoal (50% water wet, 370 mg) in EtOH (100 mL), was hydrogenated under 4 bar at room temperature for 5 hours. The resulting mixture was filtered and the filtrate was concentrated to afford tert-butyl 4-(4-(tert-butoxycarbonylamino)-3-methoxyphenyl)piperidine-1-carboxylate (3.57 g, 96%) as a white foam. This product (1 g, 2.46 mmol) was stirred at 25° C. for 1 hour in a 4M solution of hydrogen chloride in dioxane (12 mL). The white precipitate was filtered, washed twice with diethyl ether and dried under vacuum at room temperature to afford 2-methoxy-4-(piperidin-4-yl)aniline dihydrochloride (0.655 g, 95%) as a white solid. This product (655 mg, 2.35 mmol) was suspended in dichloromethane (5 mL) and neutralised with DBU (0.87 mL, 5.8 mmol). 1,3-Diacetylbenzimidazol-2-one (Heterocycles, 2000, vol. 53, p. 529, 512 mg, 2.35 mmol) was then added and the solution was stirred at room temperature for 1 hour. The reaction mixture was quenched with water, extracted with dichloromethane and the combined organic layers were washed with brine, dried over magnesium sulfate and concentrated. The crude product was purified by flash chromatography on silica gel eluting with 0 to 80% ethyl acetate in dichloromethane to afford 1-(4-(4-amino-3-methoxyphenyl)piperidin-1-yl)ethanone (310 mg, 53%); NMR spectrum: 1.32-1.43 (m, 1H), 1.46-1.58 (m, 1H), 1.66-1.78 (m, 2H), 2.01 (s, 3H), 2.50-2.62 (m partially hidden by DMSOd5. 2H), 3.03-3.12 (m, 1H), 3.74 (s, 3H), 3.84-3.92 (m, 1H), 4.49 (bs, 2H), 4.51 (bs, 1H), 6.47-6.57 (m, 2H), 6.66 (s, 1H).

### Method 13

### Preparation of 4-(4-amino-3-methoxyphenyl)pyrrolidin-2-one

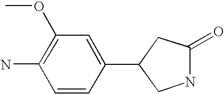

(1,1′-bis-(diphenylphosphino)-ferrocene) palladium dichloride (34.0 mg, 0.04 mmol) and cesium carbonate (821 mg, 2.52 mmol) in water (1 ml) were added to a stirred solution of tert-butyl (2-methoxy-4-(4,4,5,5-tetramethyl-1,3,2-dioxaborolan-2-yl)phenyl)carbamate (US2005/020619, 440 mg, 1.26 mmol) and tert-butyl 2-oxo-4-(tosyloxy)-2,5-dihydro-1H-pyrrole-1-carboxylate (Journal of Organic Chemistry, 2002, vol. 67, p. 4702, 297 mg, 0.84 mmol) in THF (10 ml) under argon. The resulting solution was stirred at 25° C. for 45 min then reflux for 3 hours. After cooling, the mixture was filtered and evaporated to dryness. The crude product was taken up in EtOAc, washed with a saturated solution of bicarbonate, then brine. After evaporation, the residue was purified by flash chromatography on silica gel eluting with 0 to 40% EtOAc/petroleum ether to afford tert-butyl 4-(4-(tert-butoxycarbonylamino)-3-methoxyphenyl)-2-oxo-2,5-dihydro-1H-pyrrole-1-carboxylate (283 mg, 83%) as a white solid.

TFA (4 ml) was added to a solution of tert-butyl 4-(4-(tert-butoxycarbonylamino)-3-methoxyphenyl)-2-oxo-2,5-dihydro-1H-pyrrole-1-carboxylate (267 mg, 0.66 mmol) in dichloromethane (4 mL). The solution was stirred for 3 hours then evaporated to dryness. The residue was taken up in dichloromethane and washed with a saturated aqueous solution of sodium bicarbonate. The organic layer was dried (MgSO4), filtered and evaporated to give 4-(4-amino-3-methoxyphenyl)-1H-pyrrol-2(5H)-one (124 mg, 92%) as a brown solid, which was used without further purification in the next step.

4-(4-amino-3-methoxyphenyl)-1H-pyrrol-2(5H)-one (124 mg, 0.61 mmol) in ethanol (2 mL) was hydrogenated in the presence of palladium on carbon (646 mg) under 1 atm at room temperature for 5 hours. The resulting suspension was filtered and the filtrate was concentrated to dryness. The residue was purified by flash chromatography on silica gel eluting with 0 to 15% MeOH/CH2Cl2 to afford 4-(4-amino-3-methoxyphenyl)pyrrolidin-2-one (44 mg, 35%) as a beige solid; NMR spectrum: (CDCl₃) 2.47 (dd, 1H), 2.69 (dd, 1H), 3.37 (dd, 1H), 3.57-3.66 (m, 1H), 3.70-3.76 (m, 1H), 3.82 (bs, 2H), 3.86 (s, 3H), 5.70 (bs, 1H), 6.64-6.71 (m, 3H).

### Method 14

### Preparation of 1-[2-(4-amino-3-methoxyphenyl)piperidin-1-yl]ethanone

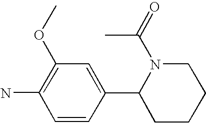

Tetrakis (triphenylphosphine) palladium (0.303 g, 0.26 mmol) was added to a stirred mixture of tert-butyl (2-methoxy-4-(4,4,5,5-tetramethyl-1,3,2-dioxaborolan-2-yl)phenyl)carbamate (US2005/020619, 2.20 g, 6.29 mmol), 2-bromopyridine (0.5 ml, 5.24 mmol) and a 2M aqueous solution of potassium carbonate (7 ml, 14 mmol) in DME (7 ml) under argon. The resulting solution was stirred at 100° C. for 20 hours. The reaction mixture was allowed to cool to room temperature, quenched with water and extracted with ethyl acetate. The organic phase was washed with brine, dried over magnesium sulfate, concentrated then purified by flash chromatography on silica gel eluting with 5 to 20% ethyl acetate in petroleum ether to afford tert-butyl N-[2-methoxy-4-(2-pyridyl)phenyl]carbamate (0.813 g, 51%); NMR spectrum: 1.48 (s, 9H), 3.91 (s, 3H), 7.31 (ddd, 1H), 7.65 (dd, 1H), 7.73 (d, 1H), 7.83-7.88 (m, 2H), 7.97 (d, 1H), 8.00 (s, 1H), 8.64 (ddd, 1H).

A suspension of tert-butyl N-[2-methoxy-4-(2-pyridyl)phenyl]carbamate (812 mg, 2.70 mmol) and platinum (IV) oxide (43 mg, 0.19 mmol) in methanol (25 mL), was hydrogenated under 4 bars at 25° C. for 20 hours. The resulting suspension was filtered, the filtrate was concentrated then dissolved in dichloromethane and washed with a diluted solution of ammonium hydroxide. The organic phase was dried over magnesium sulfate and concentrated to dryness to afford tert-butyl N-[2-methoxy-4-(2-piperidyl)phenyl]carbamate (780 mg, 94%). This product (748 mg, 2.44 mmol) was reacted with acetic anhydride (3.3 ml, 29.3 mmol) in dichloromethane (35 ml) at room temperature for 2 hours. After concentration under vacuum, the reaction mixture was quenched with water, basified with a saturated aqueous solution of sodium carbonate and extracted with ethyl acetate. The combined organic phases were washed with brine, dried over magnesium sulfate and concentrated. The crude product was purified by flash chromatography on silica gel eluting with 20% ethyl acetate in dichloromethane to afford tert-butyl N-[4-(1-acetyl-2-piperidyl)-2-methoxy-phenyl]carbamate (770 mg, 91%) as a white foam. This product (747 mg, 2.14 mmol) was dissolved in dichloromethane and TFA (2.5 ml) was added. The resulting solution was stirred at room temperature for 3 hours. After concentration under vacuum, the residue was treated with a saturated aqueous solution of sodium bicarbonate and the mixture was extracted with ethyl acetate. Purification of the crude product by flash chromatography on silica gel eluting with 40 to 60% ethyl acetate in dichloromethane afforded 1-[2-(4-amino-3-methoxy-phenyl)-1-piperidyl]ethanone (447 mg, 84%) as a white solid; NMR spectrum: 1.20-1.87 (m, 5H), 2.01 (bs, 1.5H), 2.10 (ns, 1.5H), 2.22-2.34 (m, 1H), 2.93 (bs, 0.5H), 3.65 (bs, 0.5H), 3.75 (s, 3H), 4.35 (bs, 0.5H), 4.53-4.71 (m, 2.5H), 4.99 (bs, 0.5H), 5.64 (bs, 0.5H), 6.49 (d, 1H), 6.56 (s, 1H), 6.61 (d, 1H).

### Method 15

### Preparation of 1-[4-(4-amino-3-fluoro-5-methoxyphenyl)piperazin-1-yl]ethanone

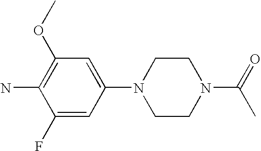

This compound was prepared following the procedure described for 1-(4-(4-amino-3-methoxyphenyl)piperazin-1-yl)ethanone (Method 7) using 1,5-difluoro-3-methoxy-2-nitrobenzene (US 2005/176726) as a starting material; NMR spectrum: 2.03 (s, 3H), 2.89-2.94 (m, 2H), 2.95-3.01 (m, 2H), 3.50-3.58 (m, 4H), 3.78 (s, 3H), 4.11 (bs, 2H), 6.52 (dd, 1H), 6.40 (dd, 1H).

### Method 16

### Preparation of 4-(4-amino-3-methoxyphenyl)-N-methylpyrrolidine-2-carboxamide

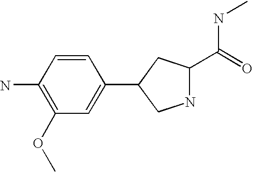

Tetrakis (triphenylphosphine) palladium (0.098 g, 0.09 mmol) was added to a stirred suspension of 1-tert-butyl 2-methyl 4-(trifluoromethylsulfonyloxy)-1H-pyrrole-1,2(2H,5H)-dicarboxylate (Organic Letters, 2006, vol. 8, p. 5665, 1.48 g, 3.55 mmol), tert-butyl (2-methoxy-4-(4,4,5,5-tetramethyl-1,3,2-dioxaborolan-2-yl)phenyl)carbamate (US2005/020619, 1.487 g, 4.26 mmol) and sodium carbonate 2M (4.26 ml, 8.52 mmol) in dioxane (30 ml) under argon. The resulting solution was stirred at 120° C. for 2 hours. The reaction mixture was allowed to cool to room temperature, diluted with EtOAc and washed with water and brine. The organic layer was dried over magnesium sulfate and concentrated to afford the crude product which was purified by flash chromatography on silica gel eluting with 0 to 20% EtOAc/petroleum ether. The solvent was evaporated to dryness to afford 1-tert-butyl 2-methyl 4-(4-(tert-butoxycarbonylamino)-3-methoxyphenyl)-1H-pyrrole-1,2(2H,5H)-dicarboxylate (1.10 g, 69%) as a white solid.

Sodium hydroxide (6M solution, 1.85 ml, 11.1 mmol) was added to a stirred solution of 1-tert-butyl 2-methyl 4-(4-(tert-butoxycarbonylamino)-3-methoxyphenyl)-1H-pyrrole-1,2(2H,5H)-dicarboxylate (1.01 g, 2.25 mmol) in methanol (20 ml). The resulting solution was stirred at 60° C. for 5 hours. After evaporation of the solvent, the resulting solid was taken up in water (10 ml) and the pH was adjusted to 6 with 6N hydrochloric acid. The aqueous layer was extracted with EtOAc and the combined the organic layers were washed with brine, dried over MgSO4, filtered and concentrated to afford 1-(tert-butoxycarbonyl)-4-(4-(tert-butoxycarbonylamino)-3-methoxyphenyl)-2,5-dihydro-1H-pyrrole-2-carboxylic acid (980 mg, 100%) as a white solid. This product was dissolved in DMF and 2-(3H-[1,2,3]triazolo[4,5-b]pyridin-3-yl)-1,1,3,3-tetramethylisouronium hexafluorophosphate (1.21 g, 3.19 mmol), N-ethyl-N,N-diisopropylamine (1.39 ml, 7.97 mmol) and methylamine hydrochloride (0.162 g, 2.39 mmol). The resulting solution was stirred at 25° C. overnight then diluted with ethyl acetate and water. The organic layer was washed with brine, dried over MgSO4, filtered, concentrated and the crude product was purified by flash chromatography on silica gel eluting with 10 to 100% EtOAc/petroleum ether to afford tert-butyl 4-(4-(tert-butoxycarbonylamino)-3-methoxyphenyl)-2-(methylcarbamoyl)-2,5-dihydro-1H-pyrrole-1-carboxylate (0.705 g, 69%) as a white solid. This product was dissolved in ethanol (10 ml) and hydrogenated under 1 atm for 5 hours in presence of 10% palladium on charcoal (160 mg). The resulting suspension was filtered and the filtrate was concentrated to dryness to afford the crude tert-butyl 4-(4-(tert-butoxycarbonylamino)-3-methoxyphenyl)-2-(methylcarbamoyl)pyrrolidine-1-carboxylate (700 mg, 100%) as a white solid; NMR spectrum: 1.33 (s, 6H), 1.39 (s, 3H) 1.44 (s, 9H), 1.78-1.89 (m, 1H), 2.43-2.51 (m partially hidden by DMSOd5. 1H), 2.57-2.33 (m, 2H), 3.19-3.32 (m partially hidden by H2O, 2H), 3.80 (s, 3H), 3.82-3.89 (m, 1H), 4.05-4.14 (m, 1H), 6.79 (dd, 1H), 6.90 (d, 1H), 7.58 (d, 1H), 7.84 (s, 1H), 7.84 (dd, 0.33H), 7.93 (dd, 0.66H).

Trifluoroacetic acid (4 mL) was added to a stirred solution of tert-butyl 4-(4-(tert-butoxycarbonylamino)-3-methoxyphenyl)-2-(methylcarbamoyl)pyrrolidine-1-carboxylate (225 mg, 0.50 mmol) in dichloromethane (3 mL) at 25° C. The resulting solution was stirred for 1 hour then concentrated to dryness and the resulting oil taken-up with dichloromethane containing 20% methanol. This solution was washed with a saturated aqueous solution of sodium bicarbonate, then brine, dried over MgSO4, filtered and concentrated to afford 4-(4-amino-3-methoxyphenyl)-N-methylpyrrolidine-2-carboxamide (205 mg, 113%) as an orange foam. This product was used without further purification; NMR spectrum: 1.84-1.96 (m, 1H), 2.61-2.69 (m, 1H), 2.71 (d, 3H), 3.10-3.20 (m, 1H), 3.41-3.52 (m, 1H), 3.59-3.68 (m, 1H), 3.83 (s, 3H), 4.20-4.29 (m, 1H), 7.76 (d, 1H), 7.87 (d, 1H), 7.94 (s, 1H), 8.45 (q, 1H), 8.82 (bs, 1H), 9.53 (bs, 1H).

### Method 17

### Preparation of 1-[3-(4-amino-3-methoxyphenyl)piperidin-1-yl]ethanone

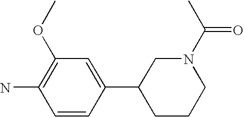

This compound was prepared following a similar procedure as described for 1-[2-(4-amino-3-methoxyphenyl)piperidin-1-yl]ethanone (Method 14) but using 3-bromopyridine instead of 2-bromopyridine.

NMR spectrum: 1.30-1.41 (m, 0.5H), 1.42-1.54 (m, 0.5H), 1.57-1.77 (m, 2H), 1.81-1.89 (m, 1H), 2.00 (s, 1.5H), 2.01 (s, 1.5H), 2.33-2.41 (m, 0.5H), 2.45-2.55 (m partially hidden by DMSOd5. 0.5H), 3.98-3.06 (m, 1H), 3.69-3.74 (m, 0.5H), 3.75 (s, 1.5H), 3.76 (s, 1.5H), 3.78-3.84 (m, 0.5H), 4.33-4.39 (m, 0.5H), 4.30-4.45 (m, 0.5H), 4.53 (s, 1H), 4.55 (s, 1H), 6.54-6.56 (m, 1H), 6.57 (s, 0.5H), 6.60 (dd, 0.5H), 6.67 (bs, 0.5H), 6.73 (d, 0.5H).

### Method 18

### Preparation of 1-[4-(4-amino-2,3-dimethylphenyl)piperazin-1-yl]ethanone

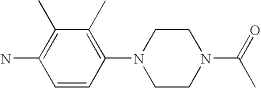

This compound was prepared following the procedure described for 1-(4-(4-amino-3-methoxyphenyl)piperazin-1-yl)ethanone (Method 7) using 1-fluoro-2,3-dimethyl-4-nitrobenzene as a starting material. In the first step, the reaction mixture was heated at 160° C. in a sealed tube; NMR spectrum: 1.96 (s, 3H), 2.02 (s, 3H), 2.17 (s, 3H), 2.52-2.76 (m, 6H), 3.52 (bs, 2H), 4.49 (bs, 2H), 6.45 (d, 1H), 6.66 (d, 1H).

### Method 19

### Preparation of 1-[4-(4-amino-2-fluoro-5-methylphenyl)piperazin-1-yl]ethanone

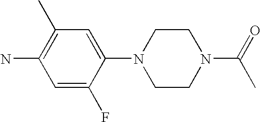

This compound was prepared following the procedure described for 1-(4-(4-amino-3-methoxyphenyl)piperazin-1-yl)ethanone (Method 7) using 1,2-difluoro-4-methyl-5-nitrobenzene as a starting material; NMR spectrum: 1.98 (s, 3H), 2.01 (s, 3H), 2.72-2.78 (m, 2H), 2.79-2.84 (m, 2H), 3.49-3.57 (m, 4H), 4.77 (bs, 2H), 6.39 (d, 1H), 6.66 (d, 1H).

### Method 20

### Preparation of 1-[4-(4-amino-3-propan-2-yloxyphenyl)piperazin-1-yl]ethanone

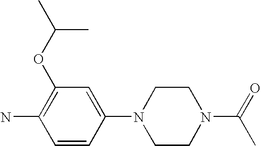

This compound was prepared following the procedure described for 1-(4-(4-amino-3-methoxyphenyl)piperazin-1-yl)ethanone (Method 7) using 4-fluoro-2-isopropoxy-1-nitrobenzene as a starting material; NMR spectrum: 1.24 (d, 6H), 2.02 (s, 3H), 2.80-2.87 (m, 2H), 2.87-2.95 (m, 2H), 3.49-3.58 (m, 4H), 4.22 (bs, 2H), 4.44-4.53 (m, 1H), 6.33 (dd, 1H), 6.52 (d, 1H), 6.54 (d, 1H).

4-fluoro-2-isopropoxy-1-nitrobenzene was prepared as follows:

2-iodopropane (3.81 mL, 38.1 mmol) was added to a stirred mixture of potassium carbonate (6.60 g, 47.7 mmol) and 5-fluoro-2-nitrophenol (3 g, 19.1 mmol) in acetone (54 mL). The resulting red thick slurry was stirred at reflux for 22 hours. The reaction mixture to was concentrated, diluted with water and extracted with ethyl acetate. The organic layer was washed with a 1M aqueous solution of sodium hydroxide, water and brine, dried over magnesium sulfate and concentrated to afford crude 4-fluoro-2-isopropoxy-1-nitrobenzene (2.27 g, 59%) as a yellow liquid. This product was used in the next step without further purification.

### Method 21

### Preparation of 1-[4-(4-amino-3-methoxyphenyl)-2-methylpiperazin-1-yl]ethanone

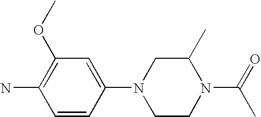

5-fluoro-2-nitroanisole (1 g, 5.84 mmol) and N,N-diisopropylethylamine (1.22 mL, 7.01 mmol) were added to a stirred solution of 2-methylpiperazine (0.644 g, 6.43 mmol) dissolved in DMA (7 mL) under argon. The resulting solution was stirred at 90° C. overnight evaporated to dryness and diluted with water (70 mL). The mixture was extracted with dichloromethane and the combined organic layers were dried over MgSO4, filtered and concentrated. The residue was triturated with diethyl ether to give a solid which was collected by filtration and dried under vacuum providing 1-(3-methoxy-4-nitrophenyl)-3-methylpiperazine (0.890 g, 60%). This product (850 mg, 3.38 mmol) was dissolved in DMA (10 ml), treated with acetic anhydride (7.53 ml, 67.6 mmol) and the mixture was heated at 70° C. for 3 hours. The solvent was evaporated, the residue was dissolved in ethyl acetate and this solution was washed with water and brine. After evaporation, the crude product was purified by flash chromatography on silica gel with 0 to 6% MeOH/CH₂Cl₂ affording 1-(4-(3-methoxy-4-nitrophenyl)-2-methylpiperazin-1-yl)ethanone (800 mg, 81%) as a yellow foam.

A solution of ammonium formate (650 mg) in water (5 ml), was added to a stirred suspension of 1-(4-(3-methoxy-4-nitrophenyl)-2-methylpiperazin-1-yl)ethanone (800 mg, 2.7 mmol) and 10% palladium on charcoal (130 mg) in MeOH (16 ml) over a period of 5 minutes at 25° C. under argon. The reaction mixture was stirred at 50° C. for 1 hour then filtered through celite and concentrated to dryness. The residue was taken in dichloromethane containing 20% methanol and this solution was washed with brine, dried over magnesium sulfate and concentrated. The crude product was purified by flash chromatography on silica gel eluting with 0 to 20% MeOH/CH₂Cl₂ to afford 1-(4-(4-amino-3-methoxyphenyl)-2-methylpiperazin-1-yl)ethanone (500 mg, 69%) as a yellow foam; NMR spectrum: 1.20 (d, 1.5H), 1.32 (d, 1.5H), 2.00 (s, 1.5H), 2.04 (s, 1.5H), 2.31-2.41 (m, 0.5H), 2.48 (bs partially hidden by DMSOd5. 0.5H), 2.52-2.59 (m, 0.5H), 2.62-2.70 (m, 0.5H), 2.87-2.97 (m, 0.5H), 3.17-3.25 (m, 1H), 3.35 (bs partially hidden by H2O, 1H), 3.38-3.44 (m, 0.5H), 3.62-3.72 (m, 0.5H), 3.75 (s, 3H), 4.24 (bs, 0.5H), 426 (bs, 2H), 4.61 (bs, 0.5H), 6.30 (dd, 1H), 6.49 (d, 1H), 6.53 (d, 1H).

### Method 22

### Preparation of 1-[4-(3-amino-2-methylphenyl)piperazin-1-yl]ethanone

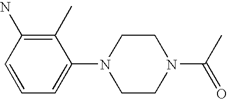

A mixture of 1-bromo-2-methyl-3-nitrobenzene (500 mg, 2.31 mmol), (9,9-dimethyl-9H-xanthene-4,5-diyl)bis(diphenylphosphine) (201 mg, 0.35 mmol), cesium carbonate (1130 mg, 3.47 mmol) and palladium (II) acetate (52 mg, 0.23 mmol) in degazed toluene (10 mL) was heated in a sealed tube at 100° C. for 12 hours. After filtration and evaporation of the filtrate, the crude product was purified by flash chromatography on silica gel eluting with 0 to 10% methanol in dichloromethane to afford 1-(4-(2-methyl-3-nitrophenyl)piperazin-1-yl)ethanone (600 mg, 98%). This product was dissolved in ethyl acetate (10 ml) and hydrogenated under 1 atm in the presence of platinum(IV) oxide (105 mg, 0.46 mmol) at 25° C. for 3 hours. After filtration, the solution was evaporated and the crude product was purified by flash chromatography on silica gel eluting with 0 to 5% MeOH/CH₂Cl₂ to afford 1-(4-(3-amino-2-methylphenyl)piperazin-1-yl)ethanone (490 mg, 91%) as a beige solid; NMR spectrum: 2.00 (s, 3H), 2.03 (s, 3H), 2.67 (bs, 2H), 2.74 (bs, 2H), 3.54 (bs, 4H), 4.78 (bs, 2H), 6.26 (d, 1H), 6.39 (d, 1H), 6.83 (dd, 1H).

### Method 23

### Preparation of 1-[4-(4-amino-3-methoxyphenyl)-2-(hydroxymethyl)piperazin-1-yl]ethanone

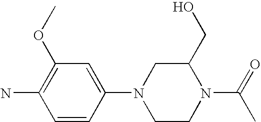

A mixture of 5-fluoro-2-nitroanisole (560 g, 3.27 mmol), piperazin-2-ylmethanol (acetate salt, 577 mg, 3.27 mmol) and N,N-diisopropylethylamine (1.71 mL, 9.82 mmol) in DMA (7 mL) was stirred at 90° C. for 25 hours. The solution was allowed to cool down to room temperature and acetic anhydride (7.29 mL, 65.4 mmol) was added. The reaction mixture was stirred for 10 minutes then evaporated and the crude product was purified by flash chromatography on silica gel eluting with 0 to 10% MeOH in dichloromethane to afford 1-(2-(hydroxymethyl)-4-(3-methoxy-4-nitrophenyl)piperazin-1-yl)ethanone (400 mg, 39%).

This product was hydrogenated in the presence of platinum (IV) oxide as described in Method 7 to afford the title compound; NMR spectrum: 2.02 (s, 1.2H), 2.06 (s, 1.8H), 2.32-2.41 (m, 1H), 2.41-2.50 (m partially hidden by H2O, 0.4H), 2.57-2.66 (m, 1H), 2.81-2.90 (m, 0.6H), 3.37-3.43 (m, 0.6H), 3.49-3.55 (m, 0.4H), 2.63-2.77 (m, 2H), 2.75 (s, 3H), 3.87-3.94 (m, 0.6H), 4.26 (bs, 2H), 4.27 (bs, 0.6H), 4.30 (bs, 0.4H), 4.39-4.46 (m, 0.4H), 4.82 (bs, 0.4H), 4.99 (bs, 0.6H), 6.27-6.34 (m, 1H), 6.48-6.56 (m, 2H).

### Method 24

### Preparation of 2-(4-amino-3-methoxyphenyl)-1-morpholin-4-ylpropan-1-one

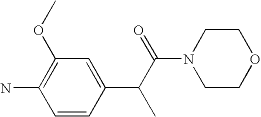

A mixture of 2-(4-amino-3-methoxyphenyl)propanoic acid (240 mg, 1.23 mmol), N,N-diisopropylethylamine (0.21 ml, 1.23 mmol), morpholine (0.108 ml, 1.23 mmol) and 2-(3H-[1,2,3]triazolo[4,5-b]pyridin-3-yl)-1,1,3,3-tetramethylisouronium hexafluorophosphate(V) (467 mg, 1.23 mmol) in dichloromethane (3 ml) was stirred at 25° C. for 3 hours. The crude product was purified by flash chromatography on silica gel eluting with 0 to 100% EtOAc in petroleum ether to afford 2-(4-amino-3-methoxyphenyl)-1-morpholinopropan-1-one (245 mg, 75%) as a beige solid; NMR spectrum: 1.23 (d, 3H), 3.02-3.11 (m, 1H), 3.22-3.29 (m, 1H), 3.25-3.56 (m, 6H), 3.72 (s, 3H), 3.87 (q, 1H), 4.58 (bs, 2H), 6.51-6.57 (m, 2H), 6.66 (s, 1H).

2-(4-amino-3-methoxyphenyl)propanoic acid was obtained as follows:

A mixture of methyl 2-(4-amino-3-methoxyphenyl)propanoate (described in Bioorganic & Medicinal Chemistry 2007, vol. 15, p. 6043, 630 mg, 3.01 mmol) and sodium hydroxide 6N (2.0 mL, 12.0 mmol) in MeOH (10 mL) was stirred at room temperature for 1 hour. Methanol was removed under vacuum and the residue was neutralised to pH 4.5 by addition of 2N hydrochloric acid. After evaporation, the residue was taken-up in dichloromethane/EtOH, the resulting suspension was filtered and the filtrate evaporated to give a foam which was triturated in ether/ethanol to give 2-(4-amino-3-methoxyphenyl)propanoic acid (485 mg, 83%) as a beige solid collected by filtration. This product was used without further purification.

Following the same procedure, 2-(4-amino-3-methoxyphenyl)-1-[4-(2-hydroxyethyl)piperazin-1-yl]propan-1-one was prepared from 2-(piperazin-1-yl)ethanol; NMR spectrum: 1.21 (d, 3H), 1.83-1.92 (m, 1H), 2.12-2.21 (m, 1H), 2.22-2.32 (m, 3H), 2.34-2.42 (m, 1H), 2.76 (bs, 2H), 3.35-3.41 (m, 1H), 3.41-3.46 (m, 2H), 3.53-3.60 (m, 1H), 3.72 (s, 3H), 3.86 (q, 1H), 4.36 (t, 1H), 4.57 (bs, 2H), 6.48-6.58 (m, 2H), 6.65 (s, 1H).

Following the same procedure, tert-butyl 4-[2-(4-amino-3-methoxyphenyl)propanoyl]piperazine-1-carboxylate was prepared from tert-butyl piperazine-1-carboxylate; NMR spectrum: (CDCl₃) 1.42 (d, 3H), 1.43 (s, 9H), 2.03 (bs, 2H), 2.68-2.77 (m, 1H), 3.08-3.17 (m, 1H), 3.24-3.54 (m, 4H), 3.52 (bs, 1H), 3.75 (q, 1H), 3.83 (s, 3H), 3.86 (bs, 1H), 6.59-6.68 (m, 3H).

### Method 25

### Preparation of 1-[(6S)-4-(4-amino-3-methoxyphenyl)-6-hydroxy-1,4-diazepan-1-yl]ethanone

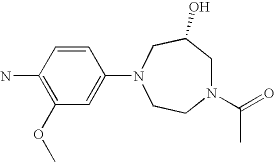

Potassium carbonate (914 mg, 6.61 mmol) was added to a mixture of 4-fluoro-2-methoxy-1-nitrobenzene (1.13 g, 6.61 mmol) and (S)-1-(2-nitrophenylsulfonyl)-1,4-diazepan-6-ol (described in Bioorganic & Medicinal Chemistry Letters 2007, vol. 17, p. 2828, 1.66 g, 5.51 mmol) in water (60 mL) and the mixture was stirred at 95° C. for 5 hours. Dioxane (40 mL) was then added until a solution was obtained and heating was continued for 4 hours. The mixture was diluted with water and extracted with ethyl acetate. The organic layer was washed with brine, dried over MgSO4, filtered and concentrated. The residual oil was purified by flash chromatography on silica gel eluting with 0 to 2% methanol in dichloromethane to afford an impure product which was purified again by flash chromatography on silica gel eluting with 0 to 25% diethylether in dichloromethane to afford (S)-1-(3-methoxy-4-nitrophenyl)-4-(2-nitrophenylsulfonyl)-1,4-diazepan-6-ol (720 mg, 29%) as a yellow solid.

Cesium carbonate (488 mg, 1.50 mmol) and polymer supported thiophenol (0.60 mmol) was added to a solution of (S)-1-(3-methoxy-4-nitrophenyl)-4-(2-nitrophenylsulfonyl)-1,4-diazepan-6-ol (226 mg, 0.50 mmol) in THF (10 mL) and the mixture was stirred and heated to 75° C. After 6 hours, the mixture was filtered and the solid washed with THF then dichloromethane and the filtrate was concentrated. The crude product was purified by flash chromatography on silica gel eluting with 0 to 8% methanol in dichloromethane to afford (R)-1-(3-methoxy-4-nitrophenyl)-1,4-diazepan-6-ol (100 mg, 745%) as a yellow foam. 1,3-Diacetylbenzimidazol-2-one (Heterocycles, 2000, vol. 53, p. 529, 237 mg, 1.09 mmol) was added to a solution of (R)-1-(3-methoxy-4-nitrophenyl)-1,4-diazepan-6-ol (290 mg, 1.09 mmol) in THF (25 mL) and the mixture was stirred under reflux for 7 hours. The mixture was filtered and the filtrate concentrated. The residue was purified by flash chromatography on silica gel eluting with 0 to 70% diethylether in dichloromethane, then with 2 to 4% of methanol in dichloromethane to afford (S)-1-(6-hydroxy-4-(3-methoxy-4-nitrophenyl)-1,4-diazepan-1-yl)ethanone (257 mg, 77%) as a yellow foam.

(S)-1-(6-hydroxy-4-(3-methoxy-4-nitrophenyl)-1,4-diazepan-1-yl)ethanone (340 mg, 1.10 mmol) was hydrogenated in the presence of platinum (IV) oxide under 1.6 bar for 30 minutes in a mixture of ethanol (25 ml) and ethyl acetate (5 ml). The resulting suspension was filtered and the filtrate was concentrated to afford (S)-1-(4-(4-amino-3-methoxyphenyl)-6-hydroxy-1,4-diazepan-1-yl)ethanone (300 mg, 98%); NMR spectrum: 1.78 (s, 1.5H), 2.03 (s, 1.5H), 2.78 (dd, 0.5H), 2.99 (dd, 0.5H), 3.06 (dd, 0.5H), 3.15-3.23 (m, 0.5H), 3.25-3.31 (m partially hidden by H2O, 0.5H), 3.34-3.38 (m partially hidden by H2O, 0.5H), 3.38-3.45 (m, 1H), 3.49-3.56 (m, 1H), 3.57-3.68 (m, 2H), 3.73 (s, 3H), 3.78-3.89 (m, 1H), 3.90-3.99 (m, 1H), 3.99 (bs, 2H), 5.00 (d, 0.5H), 5.13 (d, 0.5H), 6.16 (dd, 1H), 6.34 (d, 0.5H), 6.35 (d, 0.5H), 6.49 (d, 0.5H), 6.50 (d, 0.5H).

### Method 26

### Preparation of tert-butyl 4-(4-amino-3-methoxyphenoxy)piperidine-1-carboxylate

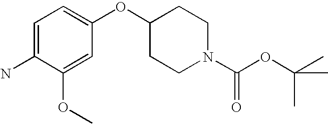

A mixture of tert-butyl 4-hydroxypiperidine-1-carboxylate (4.70 g, 23.3 mmol), 4-fluoro-2-methoxy-1-nitrobenzene (2 g, 11.6 mmol), tetrabutylammonium bromide (0.754 g, 2.34 mmol) and 25% aqueous KOH (10 ml) in toluene (10 ml) was stirred at 60° C. overnight. The reaction mixture was allowed to cool to room temperature, diluted with water and extracted with ethyl acetate. The combined organic phases were washed with water, brine, dried over magnesium sulfate, and evaporated. The crude product was purified on silica gel eluting with 20 to 35% ethyl acetate in petroleum ether to afford tert-butyl 4-(3-methoxy-4-nitrophenoxy)piperidine-1-carboxylate (3.73 g, 91%) as a yellow oil. This product (3.66 g, 10.3 mmol) was hydrogenated under 1 atm at 25° C. for 3 hours in the presence of platinum (IV) oxide (0.165 g, 0.73 mmol) in EtOH (200 ml). The suspension was filtered and the filtrate was concentrated to dryness. The crude product was purified by flash chromatography on silica gel eluting with 5% methanol in dichloromethane to afford tert-butyl 4-(4-amino-3-methoxyphenoxy)piperidine-1-carboxylate (quantitative yield) as a solid; NMR spectrum: 1.40 (s, 9H), 1.42-1.52 (m, 2H), 1.78-1.87 (m, 2H), 3.09-3.21 (m, 2H), 3.59-3.67 (m, 2H), 3.72 (s, 3H), 3.23-3.33 (m, 3H), 6.34 (dd, 1H), 6.48 (d, 1H), 6.52 (d, 1H).

Using the same procedure, tert-butyl (3R)-3-(4-amino-3-methoxyphenoxy)pyrrolidine-1-carboxylate and tert-butyl (3S)-3-(4-amino-3-methoxyphenoxy)pyrrolidine-1-carboxylate have been prepared using respectively (R)-tert-butyl 3-hydroxypyrrolidine-1-carboxylate and (S)-tert-butyl 3-hydroxypyrrolidine-1-carboxylate instead of tert-butyl 4-hydroxypiperidine-1-carboxylate; NMR spectrum: 1.39 (s, 4.5H), 1.40 (s, 4.5H), 1.95-2.09 (m, 2H), 3.26-3.46 (m partially hidden by H2O, 4H), 3.73 (s, 3H), 4.29 (ns, 2H), 4.76-4.84 (m, 1H), 6.29 (dd, 1H), 6.45 (d, 1H), 6.54 (d, 1H).

### Method 27

### Preparation of tert-butyl (2R)-2-[(4-amino-3-methoxyphenoxy)methyl]pyrrolidine-1-carboxylate

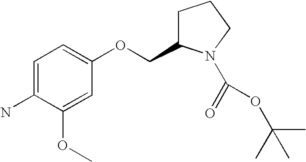

A solution of di-tert-butyl azodicarboxylate (2.45 g, 10.6 mmol) in THF (10 ml) was added dropwise to a stirred solution of 3-methoxy-4-nitrophenol (Organic Letters, 2008, vol. 10, p. 997, 600 mg, 3.55 mmol), (R)-tert-butyl 2-(hydroxymethyl)pyrrolidine-1-carboxylate (1.07 g, 5.32 mmol) and triphenylphosphine (2.79 g, 10.6 mmol) in THF (7 ml) over a period of 15 minutes at −10° C. under argon. The resulting suspension was stirred at room temperature overnight. The reaction mixture was concentrated to dryness and the residue purified by flash chromatography on silica gel eluting with 1 to 3% ethyl acetate in dichloromethane to afford (R)-tert-butyl 2-((3-methoxy-4-nitrophenoxy)methyl)pyrrolidine-1-carboxylate (440 mg, 35%) as a yellow viscous oil. This product (425 mg, 1.21 mmol) was dissolved in EtOH (13 ml) and hydrogenated under 1 atm at 25° C. in the presence of platinum (IV) oxide for 2 hours. The suspension was filtered and the filtrate was concentrated to dryness then purified by flash chromatography on silica gel eluting with 10 to 15% ethyl acetate in dichloromethane to afford (R)-tert-butyl 2-((4-amino-3-methoxyphenoxy)methyl)pyrrolidine-1-carboxylate (332 mg, 85%) as a pale red oil; NMR spectrum: 1.40 (s, 9H) 1.73-1.83 (m, 1H), 1.84-2.0 (m, 4H), 3.22-3.31 (m, 2H), 3.74 (s, 3H), 3.74-3.79 (m, 1H), 3.91-3.99 (m, 2H), 4.15 (bs, 2H), 6.31 (dd, 1H), 6.47 (bs, 2H), 6.53 (d, 1H).

Using the same procedure, tert-butyl (2S)-2-[(4-amino-3-methoxyphenoxy)methyl]pyrrolidine-1-carboxylate has been prepared starting from (S)-tert-butyl 2-(hydroxymethyl)pyrrolidine-1-carboxylate.

Using the same procedure, tert-butyl (2S)-2-[(3-amino-4-methoxyphenoxy)methyl]pyrrolidine-1-carboxylate and tert-butyl (2R)-2-[(3-amino-4-methoxyphenoxy)methyl]pyrrolidine-1-carboxylate have been prepared starting respectively from (S)-tert-butyl 2-(hydroxymethyl)pyrrolidine-1-carboxylate and (R)-tert-butyl 2-(hydroxymethyl)pyrrolidine-1-carboxylate, and using 4-methoxy-3-nitrophenol (described in WO2005/016894) instead of 3-methoxy-4-nitrophenol; NMR spectrum: 1.40 (s, 9H) 1.72-1.83 (m, 1H), 1.84-2.02 (m, 3H), 3.22-3.29 (m, 2H), 3.68 (s, 3H), 3.68-3.73 (ms, 1H), 3.86-3.92 (m, 1H), 4.92-4.98 (m, 1H), 4.71 (bs, 2H), 6.09 (dd, 1H), 6.24 (d, 1H), 6.64 (d, 1H).

### Method 28

### Preparation of tert-butyl 3-(4-amino-3-methoxyphenoxy)azetidine-1-carboxylate

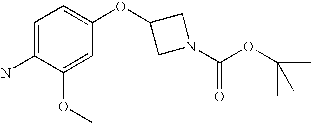

A mixture of 4-fluoro-2-methoxy-1-nitrobenzene (500 mg, 2.92 mmol), tert-butyl 3-hydroxyazetidine-1-carboxylate (506 mg, 2.92 mmol), tetrabutylammonium bromide (188 mg, 0.58 mmol) and an aqueous solution of potassium hydroxide (5N, 2.4 ml) in toluene (2.5 ml) was stirred at 60° C. for 15 hours. The reaction mixture was cooled to room temperature, poured into water and extracted with ethyl acetate. The organic layers were combined and evaporated. The crude product was purified by flash chromatography on silica gel eluting with 10 to 70% ethyl acetate in petroleum ether to afford tert-butyl 3-(3-methoxy-4-nitrophenoxy)azetidine-1-carboxylate (720 mg, 76%) as a pale yellow gum. This product was hydrogenated under 1.3 bar at 25° C. for 90 minutes in the presence of platinum (II) oxide (39 mg, 0.17 mmol) in EtOH (30 ml). The resulting suspension was filtered and the filtrate was concentrated to dryness to afford tert-butyl 3-(4-amino-3-methoxyphenoxy)azetidine-1-carboxylate (591 mg, 91%) as a colourless gum; NMR spectrum: 1.38 (s, 9H), 3.74 (s, 3H), 3.75 (bs, 2H), 4.17-4.26 (m, 2H), 4.31 (bs, 2H), 4.80-4.87 (m, 1H), 6.14 (dd, 1H), 6.39 (d, 1H), 6.53 (d, 1H).

### Method 29

### Preparation of 1-(4-(4-amino-3-methoxyphenyl)-2-methylpiperidin-1-yl)ethanone

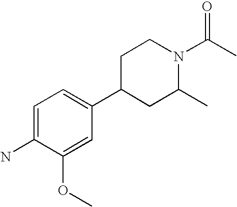

This compound was prepared using a similar procedure as for Method 14 starting with 4-bromo-2-methylpyridine instead of 2-bromopyridine; NMR spectrum: 1.09 (s, 0.75H), 1.10 (s, 0.75H), 1.49-1.57 (m, 1H), 1.59-1.68 (m, 1H), 1.74-1.83 (m, 1H), 2.00 (s, 3H), 2.03 (bs, 1H), 2.44-2.50 (m partially hidden by DMSOd5. 1H), 2.52-2.60 (m, 2H), 3.74 (s, 3H), 4.10 (bs, 1H), 4.49 (bs, 2H), 6.51-6.57 (m, 2H), 6.69 (s, 1H).

### Method 30

### Preparation of tert-butyl 4-(3-amino-4-methoxyphenyl)piperidine-1-carboxylate

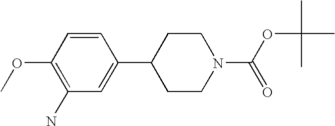

A mixture of tert-butyl 4-(4,4,5,5-tetramethyl-1,3,2-dioxaborolan-2-yl)-3,6-dihydropyridine-1(2H)-carboxylate (see Tetrahedron Letters 2000, vol. 41, p. 3705, 2.0 g, 6.46 mmol), 4-bromo-1-methoxy-2-nitrobenzene (1.5 g, 6.46 mmol), {1,1′-bis(diphenylphosphene)ferrocene}palladium chloride (complex in CH₂Cl₂, 0.209 g, 0.26 mmol) and potassium carbonate (2.68 g, 19.3 mmol) in DMF (25 ml) was degassed with nitrogen and stirred at 80° C. for 16 hours. The reaction mixture was diluted with EtOAc and washed with water. The organic layer was evaporated and the crude product was purified by flash chromatography on silica gel eluting with 0 to 25% ethyl acetate in petroleum ether to afford tert-butyl 4-(4-methoxy-3-nitrophenyl)-5,6-dihydropyridine-1(2H)-carboxylate (2.06 g, 96%). This product was hydrogenated at 60 psi in the presence of 5% palladium on charcoal (50% wet, 1.01 g) in ethyl acetate (50 ml) and methanol (100 ml) for 8 hours. The catalyst was filtered through a pad of Celite and the filtrate was evaporated to dryness to yield tert-butyl 4-(3-amino-4-methoxyphenyl)piperidine-1-carboxylate (1.78 g, 97%); NMR spectrum: 1.31-1.41 (m, 2H), 1.41 (s, 9H), 1.64-1.71 (m, 2H), 2.42-2.50 (m, 1H), 2.76 (m, 2H), 3.71 (s, 3H), 3.97-4.09 (m, 2H), 4.66 (bs, 2H), 6.37 (dd, 1H), 6.50 (d, 1H), 6.68 (d, 1H).

### Method 31

### Preparation of 2-methoxy-4-(1-methylpyrrolidin-3-yl)oxyaniline

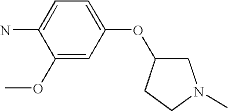

25% KOH aqueous solution (20 mL), 1-methyl-3-pyrrolidiniol (5.0 g, 50 mmol) and tetra-n-butylammonium bromide (1.66 g, 5 mmol) were added to a stirred solution of 4-iii fluoro-2-methoxy-1-nitrobenzene (4.28 g, 25 mmol) in toluene (20 mL). The resulting solution was stirred at 60° C. overnight. The mixture was allowed to cool to room temperature, poured onto ice-water (200 mL) and extracted with EtOAc (3×100 mL). The organic layer was then washed with 2 M aqueous hydrochloric acid solution (250 ml) and purified by ion exchange chromatography, using an SCX column. The desired product was eluted from the column using 7M NH3/MeOH and evaporated to dryness to afford 3-(3-methoxy-4-nitrophenoxy)-1-methylpyrrolidine (5.50 g, 87%) as a yellow oil, which solidified on standing; NMR spectrum: (400 MHz) 1.73-1.84 (m, 1H), 2.27 (s, 3H), 2.31-2.37 (m, 2H), 2.65 (dd, 1H), 2.68-2.73 (m, 1H), 2.77 (dd, 1H), 3.92 (s, 3H), 5.02-5.07 (m, 1H), 6.62 (dd, 1H), 6.73 (d, 1H), 7.95 (d, 1H); Mass spectrum: 253 (MH+).

3-(3-Methoxy-4-nitrophenoxy)-1-methylpyrrolidine (5.50 g, 20.6 mmol) and 10% Pd/C (500 mg) in ethanol (125 ml) were stirred under an atmosphere of hydrogen at ambient temperature and pressure overnight. The resulting suspension was filtered and the filtrate was concentrated to dryness to afford 2-methoxy-4-(1-methylpyrrolidin-3-yl)oxyaniline (4.74 g, quantitative yield) as a pink/red oil; NMR spectrum: (400 MHz, CDCl₃) 1.94-2.02 (m, 1H), 2.22-2.30 (m, 1H), 2.36-2.45 (m, 1H), 2.38 (s, 3H), 2.73-2.83 (m, 3H), 3.50 (s, 2H), 3.81 (s, 3H), 4.74 (octet, 1H), 6.26 (dd, 1H), 6.45 (d, 1H), 6.61 (d, 1H); Mass spectrum: 223 (MH+).

### Method 32

### Preparation of 2-methoxy-4-(4-methylpiperazin-1-yl)aniline

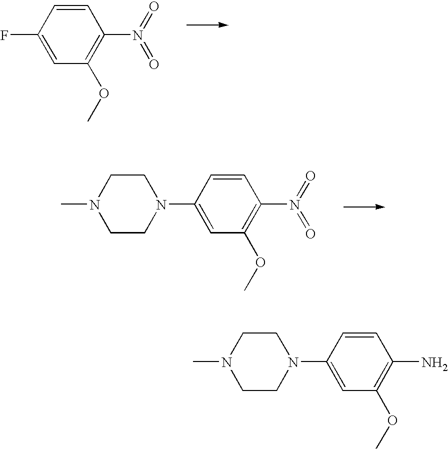

N,N-diisopropylethylamine (58.8 ml, 356 mmol) and 1-methylpiperazine (36.2 ml, 326 mmol) were added to a stirred solution of 4-fluoro-2-methoxy-1-nitrobenzene (50.73 g, 296 mmol) in DMA (400 ml). The resulting solution was stirred at 90° C. for 20 hours under nitrogen. The mixture was allowed to cool to room temperature then concentrated. The residue was dissolved in dichloromethane (1000 ml) and washed with water (600 ml). The organic layer was concentrated, then re-dissolved in EtOAc. Isohexane was added to induce crystallisation. The resulting precipitate was collected by filtration and dried under vacuum to afford 1-(3-methoxy-4-nitrophenyl)-4-methylpiperazine (56.55 g, 76%) as a yellow crystalline solid; NMR spectrum: (400 MHz) 2.24 (3H, s), 2.42-2.46 (4H, m), 3.42-3.48 (4H, m), 3.92 (3H, s), 6.54 (1H, d), 6.59-6.62 (1H, m), 7.89 (1H, d).

A suspension of 1-(3-methoxy-4-nitrophenyl)-4-methylpiperazine (56.41 g, 224 mmol) and 5% palladium on carbon (0.5 g) in ethanol (1000 ml) was hydrogenated under 3 bar at 25° C. for 18 hours. The resulting suspension was filtered through celite, then the filtrate was concentrated to afford 2-methoxy-4-(4-methylpiperazin-1-yl)aniline (48.06 g, 97%) as a dark purple oil which solidified on standing; NMR spectrum: (400 MHz) 2.22 (3H, s), 2.42-2.46 (4H, m), 2.92-2.96 (4H, m), 3.75 (3H, s), 4.20 (2H, s), 6.28-6.31 (1H, m), 6.49-6.50 (1H, m), 6.53 (1H, d).

2-Methoxy-4-(4-ethylpiperazin-1-yl)aniline was prepared from 1-ethylpiperazine using the above procedure.

1-(3-methoxy-4-nitrophenyl)-4-ethylpiperazine; NMR spectrum: (400 MHz) 1.05 (3H, t), 2.38 (2H, q), 2.46-2.50 (4H, m), 3.42-3.46 (4H, m), 3.92 (3H, s), 6.54 (1H, d), 6.59-6.62 (1H, m), 7.89 (1H, d).

2-Methoxy-4-(4-ethylpiperazin-1-yl)aniline; NMR spectrum: (400 MHz) 1.03 (3H, t), 2.36 (2H, q), 2.46-2.49 (4H, m), 2.93-2.96 (4H, m), 3.75 (3H, s), 4.22 (2H, s), 6.29-6.30 (1H, m), 6.49-6.54 (2H, m).

### Method 33

### Preparation of 2-ethoxy-4-(4-methylpiperazin-1-yl)aniline

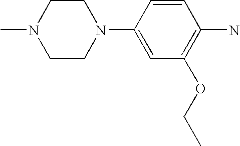

To a solution of 2-hydroxy-4-fluoronitrobenzene (1.58 g, 10 mmol) in acetone (25 mL) was added K₂CO₃ (1.8 g, 13 mmol) and the reaction was stirred at ambient temperature for 15 minutes. Ethyl iodide (0.88 mL, 11 mmol) was added and the reaction heated at reflux for 16 hours. The reaction was cooled to ambient temperature and EtOAc (20 mL) and NH₄Cl (sat. aq., 20 mL) were added. The phases were separated and the aqueous phase was extracted with EtOAc (50 mL). The combined organic phases were washed with NaOH (2N aq., 50 mL), brine (50 mL) and dried (MgSO₄). The volatiles were removed under reduced pressure to afford 2-ethoxy-4-fluoronitrobenzene (1.63 g, 88%) as a yellow solid; NMR spectrum: (400 MHz) 1.35 (t, 3H), 4.22 (q, 2H), 6.95 (m, 1H), 7.28 (dd, 1H), 8.00 (dd, 1H).

To a solution of 2-ethoxy-4-fluoronitrobenzene (1.61 g, 8.7 mmol) in NMP (20 mL) was added 1-methylpiperazine (1.06 mL, 9.6 mmol) and DIPEA (3.6 mL, 21.8 mmol). The mixture was heated at 80° C. for 20 hours, then cooled to ambient temperature. The crude mixture was loaded onto a SCX-2 column and the column was eluted with MeOH (200 mL), and the crude product eluted with NH₃ (200 mL, 7N in MeOH) and concentrated to yield a yellow solid. This solid was triturated (EtOAc:isohexane, 1:20, 50 mL) and filtered to afford 2-ethoxy-4-(4-methylpiperazin-1-yl)nitrobenzene (1.36 g, 59%) as a yellow solid; NMR spectrum: (400 MHz) 1.35 (t, 3H), 2.22 (s, 3H), 2.45 (m, 4H), 3.42 (m, 4H), 4.2 (q, 2H), 6.52 (d, 1H), 6.6 (d, 1H), 7.85 (d, 1H).

A suspension of 2-ethoxy-4-(4-methylpiperazin-1-yl)nitrobenzene (8.5 g, 32 mmol) and 5% Pd on carbon (0.85 g) in ethanol (90 ml) was hydrogenated under 5 bars at 25° C. for 16 hours. The resulting suspension was filtered through celite washing with EtOH. The volatiles were removed under reduced pressure to afford 2-ethoxy-4-(4-methylpiperazin-1-yl)aniline (7.03 g, 93%) as a purple oil; NMR spectrum: (400 MHz) 1.35 (t, 3H), 2.20 (s, 3H), 2.42 (m, 4H), 2.95 (m, 4H), 4.00 (q, 2H), 6.3 (dd, 1H), 6.47 (d, 1H), 6.55 (d, 1H).

### Method 34

### Preparation of [3-(2,5-dichloropyrimidin-4-yl)imidazo[1,2-a]pyridin-7-yl]methanol

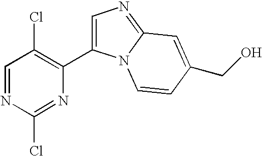

N-bromosuccinimide (2.09 g, 11.7 mmol) was added in one portion to a stirred solution of (E)-4-(2-butoxyvinyl)-2,5-dichloropyrimidine (see Method 1, 2.9 g, 11.7 mmol) in dioxane (30 mL)/water (10 mL) at 25° C. The resulting solution was stirred for 1 hour then (2-aminopyridin-4-yl)methanol (1.45 g, 11.7 mmol) was added and the solution was stirred at 85° C. for 4 hours. Solvents were evaporated and the crude product was purified by flash chromatography on silica gel eluting with 0 to 10% methanol in dichloromethane to afford (3-(2,5-dichloropyrimidin-4-yl)imidazo[1,2-a]pyridin-7-yl)methanol (2.80 g, 81%) as a yellow solid; NMR spectrum: 4.66 (d, 2H), 5.59 (t, 1H), 7.24 (dd, 1H), 7.73 (s, 1H), 8.85 (s, 1H), 8.90 (s, 1H), 9.56 (d, 1H).

The following compounds were prepared using a similar procedure, starting from the appropriately substituted 2-aminopyridine:

| Compound Name | Starting aminopyridine | NMR Spectrum |
|---|---|---|
| 6-bromo-3-(2,5- | 5-bromopyridin-2-amine | 7.78 (dd, 1H), 7.87 (d, 1H), |
| dichloropyrimidin-4- | | 8.85 (s, 1H), 8.97 (s, 1H), 9.81 (d, 1H) |
| yl)imidazo[1,2-a]pyridine | | |
| 3-(2,5-dichloropyrimidin-4- | 6-aminopyridine-3- | 7.90 (dd, 1H), 8.04 (dd, 1H), |
| yl)imidazo[2,1-f]pyridine- | carbonitrile | 8.92 (s, 1H), 9.03 (s, 1H), 10.05 (dd, |
| 6-carbonitrile | | 1H) |
| 3-(2,5-dichloropyrimidin-4- | 5-fluoropyridin-2-amine | 7.77 (ddd, 1H), 7.97 (dd, 1H), |
| yl)-6-fluoroimidazo[1,2- | | 8.90 (s, 1H), 8.97 (s, 1H), 9.66 (dd, 1H) |
| a]pyridine | | |
| 3-(2,5-dichloropyrimidin-4- | 3,5-difluoropyridin-2- | 7.98 (ddd, 1H), 8.90 (s, 1H), |
| yl)-6,8- | amine | 9.02 (s, 1H) 9.50 (dd, 1H) |
| difluoroimidazo[1,2- | | |
| a]pyridine | | |
| 3-(2,5-dichloropyrimidin-4- | 5-methylpyridin-2-amine | 2.41 (s, 3H), 7.53 (dd, 1H), |
| yl)-6-methylimidazo[1,2- | | 7.80 (d, 1H), 8.81 (s, 1H), 8.92 (s, 1H), |
| a]pyridine | | 9.45 (bs, 1H) |
| 3-(2,5-dichloropyrimidin-4- | N′,N′-dimethylpyridine- | 2.96 (s, 6H), 7.57 (dd, 1H), |
| yl)-N,N- | 2,5-diamine | 7.75 (d, 1H), 8.80 (s, 1H), 8.86 (s, 1H), |
| dimethylimidazo[2,1- | | 9.21 (d, 1H) |
| f]pyridin-6-amine | | |
| 3-(2,5-dichloropyrimidin-4- | 5-methoxypyridin-2- | 3.89 (s, 3H), 7.47 (dd, 1H), |
| yl)-6-methoxyimidazo[1,2- | amine | 7.82 (d, 1H), 8.83 (s, 1H), 8.92 (s, 1H), |
| a]pyridine | | 9.43 (d, 1H) |
| 3-(2,5-dichloropyrimidin-4- | 2-aminopyridine-4- | 7.60 (dd, 1H), 8.62 (dd, 1H), |
| yl)imidazo[1,2-a]pyridine- | carbonitrile | 8.95 (s, 1H), 9.02 (s, 1H), 9.57 (dd, 1H) |
| 7-carbonitrile | | |
| 3-(2,5-dichloropyrimidin-4- | 4-ethylpyridin-2-amine | 0.86 (t, 3H), 2.77 (q, 2H), 7.24 (dd, |
| yl)-7-ethylimidazo[1,2- | | 1H), 7.68 (dd, 1H), 8.85 (s, 1H), |
| a]pyridine | | 8.89 (s, 1H), 9.55 (d, 1H) |
| 3-(2,5-dichloropyrimidin-4- | 4-fluoropyridin-2-amine | 7.41 (ddd, 1H), 7.80 (dd, 1H), |
| yl)-7-fluoroimidazo[1,2- | | 8.86 (s, 1H), 8.94 (s, 1H), 9.65 (dd, 1H) |
| a]pyridine | | |
| 3-(2,5-dichloropyrimidin-4- | 4-methylpyridin-2-amine | 2.47 (s, 3H), 7.19 (dd, 1H), 7.68 (s, |
| yl)-7-methylimidazo[1,2- | | 1H), 8.84 (s, 1H), 8.89 (s, 1H), |
| a]pyridine | | 9.53 (d, 1H) |
| 3-(2,5-dichloropyrimidin-4- | 4-methoxypyridin-2- | 3.9 (s, 3H), 7.05 (dd, 1H), 7.29 (d, |
| yl)-7-methoxyimidazo[1,2- | amine | 1H), 8.82 (s, 1H), 8.84 (s, 1H), |
| a]pyridine | | 9.53 (d, 1H) |
| [3-(2,5-dichloropyrimidin- | (2-aminopyridin-3- | 4.94 (d, 2H), 5.53 (t, 1H), 7.33 (dd, |
| 4-yl)imidazo[1,2-a]pyridin- | yl)methanol | 1H), 7.63 (dd, 1H), 8.82 (s, 1H), |
| 8-yl]methanol | | 8.93 (s, 1H), 9.50 (d, 1H) |
| 3-(2,5-dichloropyrimidin-4- | 2-aminopyridine-3- | 7.46 (dd, 1H), 8.32 (dd, 1H), |
| yl)imidazo[1,2-a]pyridine- | carbonitrile | 8.90 (s, 1H), 9.03 (s, 1H), 9.73 (dd, 1H) |
| 8-carbonitrile | | |
| 3-(2,5-dichloropyrimidin-4- | 3-fluoropyridin-2-amine | 7.29 (ddd, 1H), 7.57 (d, 1H), |
| yl)-8-fluoroimidazo[1,2- | | 8.84 (s, 1H), 8.99 (s, 1H), 9.40 (d, 1H) |
| a]pyridine | | |
| 3-(2,5-dichloropyrimidin-4- | 3-methylpyridin-2-amine | 2.61 (s, 3H), 7.23 (dd, 1H), |
| yl)-8-methylimidazo[1,2- | | 7.47 (d, 1H), 8.84 (s, 1H), 8.92 (s, 1H), |
| a]pyridine | | 9.48 (d, 1H) |
| 3-(2,5-dichloropyrimidin-4- | 3-methoxypyridin-2- | 4.02 (s, 3H), 7.13 (d, 1H), |
| yl)-8-methoxyimidazo[1,2- | amine | 7.24 (dd, 1H), 8.79 (s, 1H), 8.95 (s, 1H), |
| a]pyridine | | 9.20 (d, 1H) |
| 3-(2,5-dichloropyrimidin-4- | pyridine-2,3-diamine | 5.96 (bs, 2H), 6.65 (dd, 1H), |
| yl)imidazo[1,2-a]pyridin-8- | | 7.03 (dd, 1H), 8.75 (s, 1H), 8.87 (s, 1H), |
| amine | | 8.95 (dd, 1H) |
| 7-chloro-3-(2,5- | 4-chloropyridin-2-amine | 7.44 (dd, 1H), 8.09 (d, 1H), |
| dichloropyrimidin-4- | | 8.88 (s, 1H), 8.97 (s, 1H), 9.57 (d, 1H) |
| yl)imidazo[1,2-a]pyridine | | |
| 7-bromo-3-(2,5- | 4-bromopyridin-2-amine | CDCl3: 7.24 (dd, 1H), 8.00 (d, |
| dichloropyrimidin-4- | | 1H), 8.62 (s, 1H), 8.97 (s, 1H), |
| yl)imidazo[1,2-a]pyridine | | 9.75 (d, 1H) |

### Method 35

### Preparation of 3-(2,5-dichloropyrimidin-4-yl)pyrazolo[1,5-a]pyridine

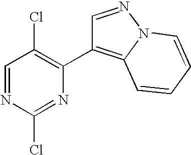

To a stirred solution of (E)-4-(2-butoxyvinyl)-2,5-dichloropyrimidine (see Method 1, 2.5 g, 10.1 mmol) in ethanol (30 mL) is added 1-aminopyridinium iodide (2.24 g, 10.1 mmol) and triethylamine (3.53 mL, 25.2 mmol). The resulting red solution is heated to reflux under air atmosphere for 5 hours. After cooling, the precipitate is filtered, washed with some ethanol and this solid is purified by chromatography on silica gel eluting with 5% AcOEt in dichloromethane to afford 3-(2,5-dichloropyrimidin-4-yl)pyrazolo[1,5-a]pyridine (0.800 g, 29%) as a white solid; NMR spectrum: (CDCl₃) 7.07 (ddd, 1H), 7.54 (ddd, 1H), 8.51 (s, 1H), 8.61 (d, 1H), 8.74 (d, 1H), 9.05 (s, 1H); Mass spectrum: 265 (MH+).

### Method 36

### Preparation of 4-(4-amino-3-methoxyphenyl)-N,N-dimethylpiperazine-2-carboxamide

O-benzotriazol-1-yl-N,N,N′,N′-tetramethyluronium tetrafluoroborate (1.14 g, 3.56 mmol) was added to a stirred mixture of 4-(3-methoxy-4-nitrophenyl)piperazine-2-carboxylic acid (obtained from piperazine-2-carboxylic acid using the procedure described in the first step of Method 7, 1 g, 3.56 mmol), N,N-diisopropylethylamine (3.10 ml, 17.7 mmol) and dimethylamine (8.89 ml, 17.7 mmol) in DMA (20 ml). The resulting suspension was stirred at 25° C. for 3 hours. After filtration and evaporation of the filtrate, the crude product was purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent. The fractions containing the desired compound were evaporated to dryness to afford 4-(3-methoxy-4-nitrophenyl)-N,N-dimethylpiperazine-2-carboxamide (0.24 g, 21%) as a yellow solid. This product was dissolved in ethanol (15 ml) and hydrogenated under 50 psi at 25° C. in the presence of palladium on charcoal (30 mg) for 3 hours. After evaporation of the solvent, the residue was purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent. The fractions containing the desired compound were evaporated to dryness to afford 4-(4-amino-3-methoxyphenyl)-N,N-dimethylpiperazine-2-carboxamide (210 mg, 97%); NMR spectrum: 1.84 (bs, 1H), 2.36 (dd, 1H), 2.40 (ddd, 1H), 2.73-2.83 (m, 1H), 2.84 (s, 3H), 2.95-3.00 (m, 1H), 3.07 (s, 3H), 3.18-3.24 (m, 1H), 3.27-3.34 (m partially hidden by H2O, 1H), 3.74 (s, 3H), 3.74-3.80 (m, 1H), 4.23 (bs, 2H), 6.29 (dd, 1H), 6.48 (d, 1H), 6.52 (d, 1H).

### Method 37

### Preparation of 2-(4-amino-3-methoxyphenyl)acetic acid

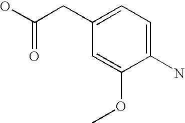

A solution of 2,2,2-trifluoroacetic anhydride (67 ml, 475 mmol) in dichloromethane (100 ml) was added dropwise to 1,4,7,10,13,16-hexaoxacyclooctadecane (5.0 g, 19.0 mmol) and tetrabutylammonium nitrate (45.5 g, 149 mmol) dissolved in dichloromethane (600 ml) over a period of 10 minutes under nitrogen at 5° C. The resulting pale yellow solution was stirred at 5° C. for 15 minutes and was added dropwise over a period of 20 minutes to a solution of 2-(3-methoxyphenyl)acetonitrile (18.4 ml, 135 mmol) dissolved in dichloromethane (1000 ml) at 5° C. The solution was stirred at room temperature for 2 hours then washed with a saturated aqueous solution of sodium bicarbonate, water, dried over MgSO4, filtered and concentrated. The crude product was purified by flash chromatography on silica gel eluting with 25 to 40% ethyl acetate in petroleum ether to afford 2-(3-methoxy-4-nitrophenyl)acetonitrile (11.5 g, 44%). This product was heated at 100° C. in 50% sulfuric acid (500 ml) for 4 hours. The resulting solution was cooled, diluted with water (500 ml) and allowed to crystallise. 2-(3-methoxy-4-nitrophenyl)acetic acid (9.07 g, 73%) was collected by filtration as a beige solid. This product (1 g, 4.74 mmol) was hydrogenated at atmospheric pressure in methanol (100 ml) in the presence of 5% palladium on charcoal during 6 hours. After filtration and evaporation 2-(4-amino-3-methoxyphenyl)acetic acid (0.845 g, 98%) was obtained as a beige solid. NMR spectrum: 3.36 (s, 2H), 3.73 (s, 3H), 4.60 (bs, sH), 6.55 (s, 1H), 6.55 (s, 1H), 6.68 (s, 1H)

### Method 38

### Preparation of 2-(4-amino-3-methoxyphenyl)propane-1,3-diol

Sodium hydride (0.514 g, 12.86 mmol) was added to a stirred solution of dimethyl malonate (1.46 ml, 12.8 mmol) in DMF (15 ml) at room temperature. After one hour 4-fluoro-2-methoxy-1-nitrobenzene (1 g, 5.84 mmol) was added and the reaction mixture was heated at 100° C. for 4 hours. The reaction was quenched with a saturated solution of ammonium chloride and extracted with ethyl acetate. The organic phase was washed with water and brine, dried over magnesium sulfate and concentrated. The crude product was purified by flash chromatography on silica gel eluting with 0 to 30% EtOAc/petroleum ether to afford dimethyl 2-(3-methoxy-4-nitrophenyl)malonate (1.16 g, 70%) as a pale yellow solid. Bromine (0.282 mL, 5.50 mmol) was added dropwise to a slurry of sodium tetrahydroborate (473 mg, 12.5 mmol) in DME (5 mL) maintained between −20° C. and −10° C. The mixture was stirred for one hour until the orange colour disappeared then the temperature was raised to −5° C. and dimethyl 2-(3-methoxy-4-nitrophenyl)malonate (708 mg, 2.50 mmol) was added. After 30 minutes, the reaction mixture was poured onto a pre-cooled mixture of 1N hydrochloric acid (7.5 ml) and EtOAc (7.5 ml). The organic phase was washed with 2N K2CO3 then water, dried (MgSO4) and concentrated. The crude product was purified by flash chromatography on silica gel eluting with EtOAc to afford 2-(3-methoxy-4-nitrophenyl)propane-1,3-diol (278 mg, 48%) as a pale colorless oil. This product was hydrogenated under 1.4 bar in ethyl acetate at 25° C. in the presence of platinum (IV) oxide (56 mg, 0.25 mmol) for 3 hours. After filtration, the solution was evaporated to dryness and the residue was triturated with ether/petroleum ether to give a solid which was collected by filtration and dried under vacuum to give 2-(4-amino-3-methoxyphenyl)propane-1,3-diol (230 mg, 95%) as a purple solid; NMR spectrum: 2.59-2.67 (m, 1H), 3.48-3.56 (m, 2H), 3.59-3.67 (m, 2H), 3.73 (s, 3H), 4.41 (t, 2H), 4.46 (bs, 2H), 6.49-6.55 (m, 2H), 6.64 (s, 1H).

### Method 39

### Preparation of 1-(4-amino-3-methoxyphenyl)piperidin-4-ol

1-(4-amino-3-methoxyphenyl)piperidin-4-ol was prepared according to the procedure described in Method 7 using 4-hydroxypiperidine as the starting amine; NMR spectrum: 1.44-1.54 (m, 2H), 1.75-1.84 (m, 2H), 2.57-2.66 (m, 2H), 3.21-3.28 (m, 2H), 3.49-3.57 (m, 1H), 3.73 (s, 3H), 4.18 (bs, 2H), 4.61 (d, 1H), 6.28 (dd, 1H), 6.48 (d, 1H), 6.49 (d, 1H).

### Method 40

### Preparation of 2-methoxy-4-(tetrahydro-2H-pyran-4-yl)aniline

In the air, (S)-pyrrolidin-2-ylmethanol (0.079 mL, 0.80 mmol), nickel chloride (dimethoxyethane adduct, 0.087 g, 0.40 mmol), 3-methoxyphenylboronic acid (2.01 g, 13.2 mmol) and potassium bis(trimethylsilyl)amide (2.65 g, 13.2 mmol) were placed in a vial equipped with a stir bar. The vial was capped and purged with argon. 2-Propanol (4 mL) was added and the resulting mixture was stirred for 5 minutes at room temperature. 4-Chlorotetrahydro-2H-pyran (0.80 g, 6.63 mmol) was then added and the vial was heated at 60° C. for 20 hours. The reaction mixture was evaporated, taken back in dichloromethane and filtered. After evaporation the crude product was purified by flash chromatography on silica gel eluting with 0 to 30% ethyl acetate in petroleum ether to afford 4-(3-methoxyphenyl)tetrahydro-2H-pyran (0.820 g, 64%) as a colorless oil; NMR spectrum: (CDCl₃) 1.62-1.73 (m, 4H), 2.58-2.67 (m, 1H), 3.37-3.45 (m, 2H), 3.70 (s, 3H), 3.94-4.00 (m, 2H), 6.65 (dd, 1H), 6.66 (d, 1H), 6.71 (d, 1H), 7.13 (dd, 1H).

A solution of 2,2,2-trifluoroacetic anhydride (2.10 ml, 14.9 mmol) in dichloromethane (5 ml) was added dropwise to 1,4,7,10,13,16-hexaoxacyclooctadecane (0.158 g, 0.60 mmol) and tetrabutylammonium nitrate (1.42 g, 4.69 mmol) dissolved in dichloromethane (25 ml) over a period of 10 minutes under nitrogen at 5° C. The resulting pale yellow solution was stirred at 5° C. for 15 minutes and was added dropwise over 20 minutes to a solution of 4-(3-methoxyphenyl)tetrahydro-2H-pyran (0.82 g, 4.27 mmol) dissolved in dichloromethane (25 ml) at 5° C. After 2 hours at room temperature, the reaction mixture was washed with a saturated aqueous solution of sodium bicarbonate, dried over MgSO₄, filtered and concentrated. The crude product was purified by flash chromatography on silica gel eluting with 0 to 30% ethyl acetate in petroleum ether to afford 4-(3-methoxy-4-nitrophenyl)tetrahydro-2H-pyran (0.420 g, 41%). This product (375 mg, 1.58 mmol) was hydrogenated at 60 psi for 15 hours in the presence of 5% palladium on charcoal (50% wet, 336 mg) in ethyl acetate (2 ml) and methanol (4.5 ml). The catalyst was filtered and the filtrate was evaporated to dryness to yield 2-methoxy-4-(tetrahydro-2H-pyran-4-yl)aniline (quantitative); NMR spectrum: 1.55-1.67 (m, 4H), 2.54-2.62 (m, 1H), 3.35-3.43 (m, 2H), 3.75 (s, 3H), 3.88-3.95 (m, 2H), 4.49 (bs, 2H), 6.52-6.57 (m, 2H), 6.67 (s, 1H).

### Method 41

### Preparation of N-(4-(4-acetylpiperazin-1-yl)-2-methoxyphenyl)formamide

A mixture of 1-(4-(4-amino-3-methoxyphenyl)piperazin-1-yl)ethanone (Method 7, 150 mg, 0.60 mmol) and methyl formate (1.48 ml, 24.0 mmol) was heated with stirring at 100° C. for 30 minutes in a microwave oven. The mixture was evaporated and the residue was purified by flash chromatography on silica gel eluting with 0 to 10% MeOH/EtOAc to afford N-(4-(4-acetylpiperazin-1-yl)-2-methoxyphenyl)formamide (126 mg, 76%) as a white solid; NMR spectrum: 2.04 (s, 3H), 3.03-3.10 (m, 2H), 3.10-3.16 (m, 2H), 3.52-3.61 (m, 4H), 3.83 (s, H), 7.47 (dd, 1H), 7.65 (d, 1H), 7.91 (d, 1H), 8.20 (d, 1H), 9.39 (s, 1H).

### Method 42

### Preparation of 4-(9-oxa-3,7-diazabicyclo[3.3.1]nonan-3-yl)-2-methoxyaniline

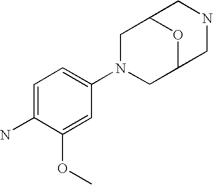

A mixture of 4-fluoro-2-methoxy-1-nitrobenzene (1.9 g, 10.0 mmol), 3-benzyl-9-oxa-3,7-diazabicyclo[3.3.1]nonane dihydrochloride (WO-2001/028992, 2.91 g, 10.0 mmol) and cesium carbonate (10.4 g, 32.0 mmol) in DMA (15 ml) was stirred at 100° C. for 3 hours. The reaction mixture was diluted with water, the orange solid was filtered, washed with water and dried under vacuum over P2O5 at 50° C. to afford crude 3-benzyl-7-(3-methoxy-4-nitrophenyl)-9-oxa-3,7-diazabicyclo[3.3.1]nonane (3.60 g, 98%) as an orange solid; NMR spectrum: 2.37-2.45 (m, 2H), 2.80-2.86 (m, 2H), 3.26 (s, 2H), 3.33-3.41 (m, 2H), 3.88 (s, 3H), 3.96-4.05 (m, 4H), 6.26 (d, 1H), 6.52 (dd, 1H), 6.95 (d, 2H), 7.11 (dd, 2H), 7.16 (t, 1H), 7.96 (d, 1H).

A suspension of 3-benzyl-7-(3-methoxy-4-nitrophenyl)-9-oxa-3,7-diazabicyclo[3.3.1]nonane (3.54 g, 9.58 mmol) and palladium on charcoal 10% (0.459 g) in ethyl acetate (25 ml) and ethanol (75 ml) was hydrogenated under 60 psi at room temperature for 16 hours. The resulting solution was filtered and the filtrate was concentrated to dryness to afford 4-(9-oxa-3,7-diazabicyclo[3.3.1]nonan-3-yl)-2-methoxyaniline (2.40 g, 100%) as a purple solid; NMR spectrum: 2.47 (bs, 1H), 2.93-3.03 (m, 4H), 3.03-3.12 (m, 2H), 3.49-3.56 (m, 2H), 3.64-3.70 (m, 2H), 3.77 (s, 3H), 4.23 (bs, 2H), 6.33 (dd, 1H), 6.56 (d, 1H), 6.59 (d, 1H).

### Method 43

### Preparation of 2-(7-(4-amino-3-methoxyphenyl)-9-oxa-3,7-diazabicyclo[3.3.1]nonan-3-yl)ethanol

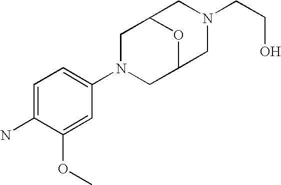

A mixture of 3-benzyl-9-oxa-3,7-diazabicyclo[3.3.1]nonane dihydrochloride (WO-2001/028992, 3 g, 10.3 mmol), 2-bromoethanol (0.845 mL, 11.3 mmol), potassium iodide (1.71 g, 10.3 mmol) and cesium carbonate (13.4 g, 41.2 mmol) in THF (35 mL) was stirred at 50° C. for 20 hours. The reaction mixture was cooled to room temperature and concentrated to dryness. The crude material was stirred in methylene chloride (50 mL) for 15 minutes and filtered. The filtrate was evaporated under reduced pressure and the crude product was purified by flash chromatography on silica gel eluting with 100% CH₂Cl₂ to a 75/25/5 mixture of CH₂Cl₂/EtOAc/NH3 7N in MeOH to afford 2-(7-benzyl-9-oxa-3,7-diazabicyclo[3.3.1]nonan-3-yl)ethanol (0.885 g, 32%) as a pale yellow oil. A suspension of 2-(7-benzyl-9-oxa-3,7-diazabicyclo[3.3.1]nonan-3-yl)ethanol (880 mg, 3.35 mmol) and palladium on charcoal (90 mg) in EtOAc (5 mL) and EtOH (30 mL, was hydrogenated under 1.7 bar at room temperature for 16 hours. The resulting suspension was filtered, washed with EtOAc and the filtrate was concentrated to dryness to afford the crude 2-(9-oxa-3,7-diazabicyclo[3.3.1]nonan-3-yl)ethanol (539 mg, 93%) as a pale yellow solid.

A mixture of 2-(9-oxa-3,7-diazabicyclo[3.3.1]nonan-3-yl)ethanol (2 g, 11.6 mmol), 4-fluoro-2-methoxy-1-nitrobenzene (1.98 g, 11.6 mmol) and cesium carbonate (4.54 g, 13.9 mmol) in DMF (15 ml) was stirred at 100° C. for 4 hours. The reaction mixture was allowed to cool to room temperature, quenched with water (75 ml) and extracted with ethyl acetate (3×100 ml). The combined organic extracts were washed with brine, dried over magnesium sulfate and concentrated to dryness to give a yellow oil which crystallized on standing. The crude product was triturated in diethyl ether, filtered and dried to afford 2-(7-(3-methoxy-4-nitrophenyl)-9-oxa-3,7-diazabicyclo[3.3.1]nonan-3-yl)ethanol (3.30 g, 88%) as a yellow solid; NMR spectrum: 2.20 (t, 2H), 2.38-2.44 (m, 2H), 2.89-2.95 (m, 2H), 3.26-3.34 (m partially hidden by H2O, 4H), 3.90 (d, 2H), 3.91 (s, 3H), 3.98-4.03 (m, 2H), 4.05 (t, 1H), 6.34 (d, 1H), 6.47 (dd, 1H), 7.90 (d, 1H).

A suspension of 2-(7-(3-methoxy-4-nitrophenyl)-9-oxa-3,7-diazabicyclo[3.3.1]nonan-3-yl)ethanol (3.3 g, 10.2 mmol) and platinum(IV) oxide (330 mg) in EtOAc (20 ml) and EtOH (65 ml) was hydrogenated under 50 psi at room temperature for 4 hours. The resulting suspension was filtered and the filtrate was concentrated to dryness to give an oil which crystallized on standing to afford 2-(7-(4-amino-3-methoxyphenyl)-9-oxa-3,7-diazabicyclo[3.3.1]nonan-3-yl)ethanol (2.92 g, 98%) as a solid; NMR spectrum: 2.27-2.35 (m, 2H), 2.43-2.50 (m partially hidden by DMSOd5, 2H), 2.85-2.95 (m, 4H), 3.40-3.49 (m, 4H), 3.75 (s, 3H), 3.93-4.00 (m, 2H), 4.08 (bs, 2H), 4.17 (bs, 1H), 6.18 (dd, 1H), 6.38 (d, 1H), 6.53 (d, 1H).

### Method 44

### Preparation of 3-(2-chloro-5-methyl-pyrimidin-4-yl)pyrazolo[1,5-a]pyridine

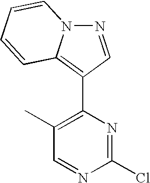

A mixture of triethylamine (31.4 ml, 225 mmol), diacetoxypalladium (3.37 g, 15 mmol), 1-(vinyloxy)butane (83 ml, 644 mmol) and 2,4-dichloro-5-methylpyrimidine (35 g, 214 mmol) in polyethylene glycol 400 (175 ml, 214 mmol) under nitrogen was stirred at 80° C. for 15 hours. The black suspension was cooled and extracted with diethyl ether (2×1000 mL). The organic layer was washed with brine (1000 mL), dried on MgSO4, filtered and evaporated to afford an orange oil. This oil was purified by flash chromatography on silica gel eluting with 15% ethyl acetate in petroleum ether to afford (E)-4-(2-butoxyvinyl)-2-chloro-5-methylpyrimidine (27.1 g, 55%); NMR spectrum: CDCl₃: 0.97 (t, 3H), 1.39-1.50 (m, 2H), 1.68-1.77 (m, 2H), 2.17 (s, 3H), 4.00 (t, 2H), 5.80 (d, 1H), 7.97 (d, 1H), 8.15 (s, 1H).

A mixture of (E)-4-(2-butoxyvinyl)-2-chloro-5-methylpyrimidine (10 g, 44.1 mmol), 1-aminopyridinium iodide (9.79 g, 44.1 mmol) and potassium carbonate (15.2 g, 110 mmol) in DMF (100 ml) was stirred at 25° C. for 16 hours. The reaction mixture was then heated at 110° C. for 2 hours. After cooling the reaction mixture was diluted in water (450 ml) and the orange solid was collected by filtration, washed with water and dried under vacuum over P2O5 at 50° C. This solid was purified by chromatography on silica gel eluting with 0 to 25% ethyl acetate in methylene chloride to afford 3-(2-chloro-5-methylpyrimidin-4-yl)pyrazolo[1,5-a]pyridine (5.20 g, 48%) as a solid; NMR spectrum: CDCl₃: 2.55 (s, 3H), 7.03 (dd, 1H), 7.48 (dd, 1H), 8.37 (s, 1H), 8.45 (s, 1H), 8.58 (d, 1H), 8.74 (d, 1H).

### Method 45

### Preparation of 3-(2-chloro-5-fluoro-pyrimidin-4-yl)pyrazolo[1,5-a]pyridine

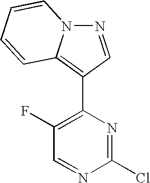

This compound was prepared following the procedure described for Method 44, using 2,4-dichloro-5-fluoropyrimidine instead of 2,4-dichloro-5-methylpyrimidine as the starting materials; NMR spectrum: CDCl3: 7.07 (ddd, 1H), 7.53 (ddd, 1H), 8.38 (d, 1H), 8.61 (d, 1H), 8.64 (d, 1H), 8.74 (s, 1H).

### Method 46

### Alternative Preparation of 3-(2-chloro-5-fluoropyrimidin-4-yl)imidazo[1,2-a]pyridine

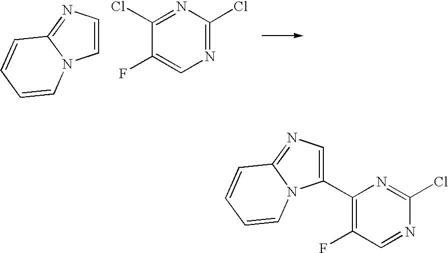

Imidazo[1,2-a]pyridine (0.51 ml, 5 mmol) was added in one portion to a stirred suspension of silver carbonate (1.93 g, 7 mmol), tris(dibenzylidineacetone)dipalladium (0) (0.114 g, 0.125 mmol) and triphenylphosphine (0.131 g, 0.50 mmol) in toluene (5 ml) at 20° C. under argon. The resulting suspension was warmed to 85° C. over 20 minutes. A solution of 2,4-dichloro-5-fluoropyrimidine (1.09 g, 6.5 mmol) in toluene (7.5 ml) was added dropwise to the suspension over a period of 2 hours. The suspension was stirred at 85° C. for 15 hours. The reaction was cooled to 50° C. and quenched dropwise with 6N HCl (5 mL) prior to diluting further with 6 M HCl (45 mL). The suspension was stirred for at least 20 minutes and then cooled to 40° C. The suspension was filtered and washed with 6M HCl (5 mL). The wash and filtrate were separated and the aqueous phase was basified to approx pH10 with 40 wt % NaOH (approx 15 mL), forming a white suspension. The precipitate was collected by filtration, washed with water (13 mL) and dried under air to afford the title compound (803 mg, 65%) as a white powder.

MS (ESI) 249 (M+H)⁺

¹H NMR spectrum: DMSO: 7.36 (1H, t), 7.65 (1H, t), 7.88 (1H, d), 8.52 (1H, d), 8.85 (1H, d), 9.75 (1H, d).

### Method 47

### Alternative Method for the Preparation of 3-(2-chloro-5-fluoropyrimidin-4-yl)imidazo[1,2-a]pyridine

To a solution of (E)-4-(2-butoxyvinyl)-2-chloro-5-fluoropyrimidine (85 g, 366.76 mmol) in dioxane (430 mL) and water (170 mL) was added N-Bromosuccinimide (65.3 g, 366.76 mmol) and the resulting mixture was allowed to stir for 60 minutes at room temperature under nitrogen before adding 2-Aminopyridine (34.5 g, 366.76 mmol) and stirring at 65° C. for 90 minutes. Reaction mixture allowed to cool giving a yellow precipitate. The dioxane was evaporated, the resulting aqueous solution diluted with water (300 mL) before pouring into aqueous NaHCO₃ (300 mL) (pH8) and stirring for 10 minutes, the resultant solid filtered, washed with water (2×250 mL) and diethyl ether (300 mL). There was thus obtained 3-(2-chloro-5-fluoropyrimidin-4-yl)imidazo[1,2-a]pyridine (54.5 g, 59.8%) as a light beige solid.

Where necessary, the product was combined with other batches prepared in a similar manner for further use.

(E)-4-(2-butoxyvinyl)-2-chloro-5-fluoropyrimidine was prepared as follows:

To a stirred solution of 2,4-dichloro-5-fluoropyrimidine (270 g, 1617.07 mmol) in Polyethylene Glycol 200 (1000 mL) under nitrogen was added triethylamine (237 mL, 1697.92 mmol), 1-(vinyloxy)butane (220 mL, 1697.92 mmol) and palladium(II) acetate (12.71 g, 56.60 mmol). This solution was allowed to stir at 80° C. for 2 hours 30 minutes before cooling to 20° C. and extracting with diethyl ether (4×1000 mL). The organics were combined before washing with water (1000 mL) and brine (1000 mL) before drying (MgSO₄) and evaporating to an orange/brown oil. The crude product was purified by flash silica chromatography, elution gradient 0 to 50% DCM in isohexane. Pure fractions were evaporated to dryness to afford (E)-4-(2-butoxyvinyl)-2-chloro-5-fluoropyrimidine (129 g, 34.5%) as a cream solid.

### Method 48

### Alternative Method for the Preparation of 2-(4-(4-amino-3-methoxyphenyl)piperazin-1-yl)ethanol

A solution of 2-(4-(3-methoxy-4-nitrophenyl)piperazin-1-yl)ethanol (238 g, 846.05 mmol) and Palladium (5% on carbon, 50% wet) (12.61 g, 2.96 mmol) in ethanol (2400 mL) was allowed to stir under a hydrogen atmosphere until theoretical amount of hydrogen had been consumed. Reaction mixture was filtered through Decalite, the filtrate then evaporated to a dark red/purple gum. Attempted to triturate material with ether (200 mL) but was unsuccessful, addition of MTBE (50 mL) and trituration once more unsuccessful. Solvent evaporated to dryness, the resultant gum then allowed to stand at room temperature overnight giving a dark purple/red solid. This material was stirred in diethyl ether (800 mL) for five hours before filtering and washing with diethyl ether (2×500 mL). 2-(4-(4-amino-3-methoxyphenyl)piperazin-1-yl)ethanol (198 g, 93%) was obtained as a purple solid.

Where necessary, the product was combined with other batches prepared in a similar manner for further use.

2-(4-(3-methoxy-4-nitrophenyl)piperazin-1-yl)ethanol was prepared as follows:

N-(2-Hydroxyethyl)piperazine (113 mL, 920.38 mmol) in DMSO (100 mL) was added to a solution of 4-fluoro-2-methoxy-1-nitrobenzene (150 g, 876.55 mmol) and Potassium carbonate (100 mL, 1753.10 mmol) in DMSO (500 mL) at room temperature. This reaction mixture was allowed to stir at room temperature for 40 minutes before stirring at 50° C. for 2 hours and 60° C. for two hours. The reaction mixture poured into water (5000 mL) giving a yellow solid precipitate which was filtered, washed with water (2×1000 mL) and diethyl ether (500 mL) before drying overnight in vacuo at 40° C. There was thus obtained 2-(4-(3-methoxy-4-nitrophenyl)piperazin-1-yl)ethanol (245 g, 99%) as a yellow solid.

4-fluoro-2-methoxy-1-nitrobenzene was prepared as follows:

5-Fluoro-2-nitrophenol (349 g, 2221.53 mmol) was suspended in acetone (3.5 L) and Potassium carbonate (0.253 L, 4443.05 mmol) added giving rise to a vivid orange/red suspension. This was heated to 40° C. and the Dimethyl sulfate (0.214 L, 2259.54 mmol) was then added and the reaction heated to reflux. The reaction colour changed to light orange with little sign of any exotherm due to rate of reflux. This was maintained at temperature for 1 hour before cooling and working up the next working day. The reaction was diluted with water (6 Lt) and EtOAC (2.5 Lt) and transferred to the separator. The aqueous was then partitioned and re-extracted with EtOAc (2×1.25 Lt). Combined organics were then washed with 50% saturated brine (1 Lt) and dried over MgSO₄, filtered, then the solvent was removed in-vacuo to give a pale yellow solid that crystallised on standing. 2 crops were isolated and analysed separately. This gave 4-fluoro-2-methoxy-1-nitrobenzene (366 g, 96%)

### X-Ray Powder Diffraction

The X-ray powder diffraction was conducted on a Siemens D5000 Instrument. The X-ray powder diffraction spectra were determined by mounting a sample of the crystalline material on a Siemens single silicon crystal (SSC) wafer mount and spreading out the sample into a thin layer with the aid of a microscope slide. The sample was spun at 30 revolutions per minute (to improve counting statistics) and irradiated with X-rays generated by a copper long-fine focus tube operated at 40 kV and 40 mA with a wavelength of 1.5406 angstroms. The collimated X-ray source was passed through an automatic variable divergence slit set at V20 and the reflected radiation directed through a 2 mm antiscatter slit and a 0.2 mm detector slit. The sample was exposed for 1 second per 0.02 degree 2-theta increment (continuous scan mode) over the range 2 degrees to 40 degrees 2-theta in theta-theta mode. The running time was 31 minutes and 41 seconds. The instrument was equipped with a scintillation counter as detector. Control and data capture was by means of a Dell Optiplex 686 NT 4.0 Workstation operating with Diffract+ software. Persons skilled in the art of X-ray powder diffraction will realise that the relative intensity of peaks can be affected by, for example, grains above 30 microns in size and non-unitary aspect ratios that may affect analysis of samples. The skilled person will also realise that the position of reflections can be affected by the precise height at which the sample sits in the diffractometer and the zero calibration of the diffractometer. The surface planarity of the sample may also have a small effect. Hence the diffraction pattern data presented are not to be taken as absolute values.

References to relative intensities are as follows:

| % Relative Intensity* | Definition |
|---|---|
| 25-100 | vs (very strong) |
| 10-25 | s (strong) |
| 3-10 | m (medium) |
| 1-3 | w (weak) |

*The relative intensities are derived from diffractograms measured with fixed slits

### Differential Scanning Calorimetry

The Differential Scanning Calorimetry was conducted on a TA Instrument Q1000 DSC. Typically less than 5 mg of material contained in a standard aluminium pan fitted with a lid was heated over the temperature range 25° C. to 325° C. at a constant heating rate of 10° C. per minute. A purge gas using nitrogen was used—flow rate 100 ml per minute.

### Biological Assays and Test Methods

### (a) Inhibition of Insulin-Like Growth Factor-1 Receptor Phosphorylation

This immunofluorescence end point cell assay measures the ability of a test compound to reduce the measured levels of IGF phosphorylation after IGF1 stimulation in R⁺ cells. R⁺ cells are derived by transfection of R⁻ mouse fibroblast cells with human IGF1R. R⁺ cells are routinely cultured in DMEM growth medium (Gibco BRL, 41966) containing 2 mM L-Glutamine (Invitrogen Code no. 25030-024) and 10% (v/v) foetal bovine serum (FBS)) in a 5% CO₂ air incubator at 37° C.

To undertake the assay, the R⁺ cells are seeded at 5×10³ cells/well in DMEM plus 1% foetal calf serum, 1% L-glutamine in 96-well black Packard View plates (PerkinElmer 6005182) and incubated at 37° C. (+5% CO₂) in a humidified incubator. The following day, the plates are dosed with 10 μl of 10× concentrated compound (diluted from 10 mM stock in DMSO and DMEM without serum) and the plates are returned to a humidified 37° C. (+5% CO₂) incubator for 30 minutes. Cells are tested in duplicates in a suitable dose range to accurately measure the compound IC50.

Following the compound treatment, the R⁺ cells are stimulated with a final concentration of 30 nM IGF1 (Gropep IM001) for 20 minutes at 37° C. The IGF1 is dissolved according to the manufacture's instructions to a 26 μM stock solution and diluted in DMEM without serum. Following stimulation the cells are fixed by adding formaldehyde (4% v/v final concentration) and incubating at room temperature for 20 minutes. The fixative solution is removed and the wells are washed twice with 100 μl phosphate buffered saline containing 0.05% Tween20 (PBS-Tween 20) before permeabilising the cells by the addition of 50 μl/well 0.05% Triton in PBS for 10 minutes at room temperature. The permeabilisation solution is removed and the cells washed twice with 100 μl/well PBS-Tween 20 before addition of 50 μl blocking solution containing 2% BSA (Sigma. A-78888)+2% goat serum (DAKO X0907) in PBS. Plates are incubated for 1 hour at room temperature. The blocking solution is aspirated from the wells and 50 μl rabbit dual phospho specific anti-phospho IGF1R/IR (BioSource 44-804) 1/350 diluted in blocking solution is added to the wells. Additionally, in-house antibodies raised against phospho IGF1R were also used at a suitable titre determined for each batch.

Following incubation at room temperature for 1 hour, the antibody solution is removed and the wells washed twice with 100 μl/well PBS-Tween 20. 50 μl/well Alexa Fluor conjugated anti rabbit (Invitrogen/Molecular Probes—A11008) is added to the wells in a dilution of 1/1000 in blocking solution. The plates are incubated at room temperature for one hour. Finally, the plates are washed three times with 100 μl/well PBS-Tween. After addition of 100 μl/well PBS the plates are sealed with a black seal.

The Green Fluorescent phospho IGF1R-associated signal in each well was measured using an Acumen Explorer HTS Reader (TTP Labtech Ltd., Cambridge). Phospho IGF1R-associated fluorescence emission can be detected at 530 nm following excitation at 488 nm. The instrument is a laser-scanning fluorescence microplate cytometer, which samples the well at regular intervals and uses threshold algorithms to identify all fluorescent intensities above the solution background without the need to generate and analyse an image. These fluorescent objects can be quantified and provide a measure of the phospho IGF1R levels in cells. Fluorescence dose response data obtained with each compound was exported into a suitable software package (such as Origin) to perform curve fitting analysis. Phospho-IGF1R levels in response to compound treatment versus stimulated and unstimulated controls were expressed as an IC₅₀ value. This was determined by calculation of the concentration of compound that was required to give a 50% reduction of the maximum phospho-IGF1R signal.

By way of example, the IC₅₀ value for 5-chloro-N-[4-(3,5-dimethylpiperazin-1-yl)-2-methoxy-phenyl]-4-imidazo[1,2-a]pyridin-3-yl-pyrimidin-2-amine in this assay is 0.004 μM.

### (b) Inhibition of Insulin Receptor Phosphorylation

This immunofluorescence end point cell assay measures the ability of a test compound to reduce the measured levels of IR phosphorylation after insulin stimulation in CHOT cells. CHOT cells are Chinese Hamster Ovary cells (CHO) stable transfected with human IR. CHOT cells are routinely cultured in Hams F12 growth medium supplemented with 200 ug/ml Geneticin, 2.5 mM HEPES, 2 mM L-Glutamine (Invitrogen Code no. 25030-024) and 10% (v/v) foetal bovine serum (FBS) in a 5% CO₂ air incubator at 37° C.

To undertake the assay, the CHOT cells are seeded at 5×10³ cells/well in Hams F12 medium plus 2.5 mM HEPES, 1% foetal calf serum and 2 mM L-Glutamine in 96-well black Packard View plates (PerkinElmer 6005182) and incubated at 37° C. (+5% CO₂) in a humidified incubator. The following day, the plates are dosed with 10 μl of 10× concentrated compound (diluted from 10 mM stock in DMSO and Hams F12 without serum) and the plates are returned to a humidified 37° C. (+5% CO₂) incubator for 30 minutes. Cells are tested in duplicates in a suitable dose range to accurately measure the compound IC50.

Following the compound treatment, the CHOT cells are stimulated with a final concentration of 30 nM Insulin (Sigma # I-9278) for 10 minutes at 37° C. The insulin is dissolved according to the manufacture's instructions to a 1.7 mM stock solution and diluted in Hams F12 medium without serum to a 113 nM solution. Following stimulation the cells are fixed by adding formaldehyde (4% v/v final concentration) and incubating at room temperature for 20 minutes. The fixative solution is removed and the wells are washed twice with 100 μl phosphate buffered saline containing 0.05% Tween20 (PBS-Tween 20) before permeabilising the cells by the addition of 50 μl/well 0.05% Triton in PBS for 10 minutes at room temperature. The permeabilisation solution is removed and the cells washed twice with 100 μl/well PBS-Tween 20 before addition of 50 μl blocking solution containing 2% BSA (Sigma. A-78888)+2% goat serum (DAKO X0907) in PBS. Plates are incubated for 1 hour at room temperature. The blocking solution is aspirated from the wells and 50 μl rabbit dual phospho specific anti-phospho IGF1R/IR (BioSource 44-804) 1/350 diluted in blocking solution is added to the wells. Additionally, in-house antibodies raised against phospho IR were also used at a suitable titre determined for each batch.

Following incubation at room temperature for 1 hour, the antibody solution is removed and the wells washed twice with 100 μl/well PBS-Tween 20. 50 μl/well Alexa Fluor conjugated anti rabbit (Invitrogen/Molecular Probes—A11008) is added to the wells in a dilution of 1/1000 in blocking solution. The plates are incubated at room temperature for one hour. Finally, the plates are washed three times with 100 μl/well PBS-Tween. After addition of 100 μl/well PBS the plates are sealed with a black seal.

The Green Fluorescent phospho IR-associated signal in each well was measured using an Acumen Explorer HTS Reader (TTP Labtech Ltd., Cambridge). Phospho IR-associated fluorescence emission can be detected at 530 nm following excitation at 488 nm. The instrument is a laser-scanning fluorescence microplate cytometer, which samples the well at regular intervals and uses threshold algorithms to identify all fluorescent intensities above the solution background without the need to generate and analyse an image. These fluorescent objects can be quantified and provide a measure of the phospho IR levels in cells. Fluorescence dose response data obtained with each compound was exported into a suitable software package (such as Origin) to perform curve fitting analysis. Phospho-IR levels in response to compound treatment versus stimulated and unstimulated controls were expressed as an IC₅₀ value. This was determined by calculation of the concentration of compound that was required to give a 50% reduction of the maximum phospho IR signal.

### (c) Enzyme Assay

For Echo dosing the solvent was 100% DMSO. A master plate was prepared with 40 ul of 10 mM stock solution of the test compound in DMSO in quadrant 1 of a Labcyte 384 well plate. A 1 in 100 dilution was made from quadrant 1 into quadrant 2 by removing 0.4 ul and adding it to 39.6 ul of DMSO. Subsequent 1 in 100 dilutions were made into quadrant 3 from quadrant 2 and quadrant 4 from quadrant 3.

Multiple 2.5 nl droplets were dispensed from each quadrant of the master plate to generate the dose range that was required in the test. The dose range most commonly used was as follows: 100 uM, 30 uM, 10 uM, 3 uM, 1 uM, 0.3 uM, 0.1 uM, 0.03 uM, 0.01 uM, 0.003 uM, 0.001 uM, 0.0001 uM. Each well was backfilled with dimethyl sulfoxide (DMSO) to a total volume of 120 nl, such that when the enzyme and substrate mix was added the final DMSO concentration was 1%.

DMSO was added to max control wells as 120 nl, minimum control wells were treated with 120 nl of compound at a concentration that inhibited the enzyme activity 100%.

### Reagent Additions:

6 ul of Enzyme mix containing: 30 nM IGFR kinase (available from Millipore); 2 mM Dithio Threitol (Sigma, UK); 10 mM Manganese Chloride (Sigma, UK); 50 mM MOPS (Sigma, UK) pH 6.5; 0.004% Triton X100 (Sigma, UK); and
a substrate mix containing 6 ul of Peptide mix containing: 3 uM peptide (Fuorescent Labelled FL-KKSRGDYMTMQIG-CONH2) and 6 uM ATP; 50 mM MOPS pH 6.5; 0.004% Triton X100 were added to each well.
It should be noted that buffers were made up with water with a specific conductance of 18 MΩ.

The mixture was incubated at room temperature for 1 hour 50 minutes before the addition of 10 ul of stop buffer which contained: 5% DMSO; 88 mM EDTA; 100 mM HEPES pH7.5; 0.033% Brij-35; 0.22% coating reagent #3 (available from Caliper). The stop solution was made up with water with a specific conductance of 18 MΩ.

Plates were read on a Caliper LabChip 3000 using the following settings: −1.2 PSI, −2000 upstream voltage, −600 downstream voltage, sample sip time of 0.2 sec, post sample sip time of 30 sec and a final delay of 120 sec.

Integration of the substrate and product peaks was carried out using Caliper LabChip software and IC50 curves were calculated using Origin™ software.

By way of example, the following Table illustrates the activity of representative compounds according to the invention in the enzyme assay (c) above.

| Example | IGFR IC₅₀ (μM) |
|---|---|
| 1 | 0.031 |
| 2 | 0.049 |
| 3.1 | 0.062 |
| 3.2 | 0.054 |
| 3.3 | 0.011 |
| 3.4 | 0.125 |
| 3.5 | 0.041 |
| 3.6 | 0.02 |
| 3.7 | 0.011 |
| 3.8 | 0.309 |
| 3.9 | 0.049 |
| 3.10 | 0.128 |
| 3.11 | 0.069 |
| 3.12 | 0.044 |
| 3.13 | 0.06 |
| 3.14 | 0.03 |
| 3.15 | 0.06 |
| 3.16 | 0.043 |
| 3.17 | 0.125 |
| 3.18 | 0.041 |
| 3.19 | 0.066 |
| 3.20 | 0.043 |
| 3.21 | 0.005 |
| 3.22 | 0.009 |
| 3.23 | 0.018 |
| 3.24 | 0.008 |
| 3.25 | 0.01 |
| 3.26 | 0.019 |
| 3.27 | 0.006 |
| 3.28 | 0.025 |
| 3.29 | 0.006 |
| 3.30 | 0.012 |
| 3.31 | 0.011 |
| 3.32 | 0.012 |
| 3.33 | 0.016 |
| 3.34 | 0.022 |
| 3.35 | 0.01 |
| 3.36 | 0.008 |
| 3.37 | 0.012 |
| 3.38 | 0.021 |
| 3.39 | 0.031 |
| 3.40 | 0.024 |
| 3.41 | 0.016 |
| 3.42 | 0.025 |
| 3.43 | 0.011 |
| 3.44 | 0.0003 |
| 3.45 | 0.022 |
| 3.46 | 0.006 |
| 3.47 | 0.032 |
| 3.48 | 0.025 |
| 3.49 | 0.019 |
| 4 | 0.003 |
| 5 | 0.01 |
| 6.1 | 0.012 |
| 6.2 | 0.014 |
| 6.3 | 0.018 |
| 6.4 | 0.012 |
| 6.5 | 0.011 |
| 6.6 | 0.014 |
| 7 | 0.016 |
| 8 | 0.02 |
| 9 | 0.021 |
| 9A | 0.018 |
| 10 | 0.02 |
| 11 | 0.018 |
| 12 | 0.022 |
| 12A | 0.013 |
| 13 | 0.02 |
| 14 | 0.019 |
| 15 | 0.012 |
| 15A | 0.036 |
| 16 | 0.032 |
| 17 | 0.058 |
| 18 | 0.06 |
| 19 | 0.011 |
| 20 | 0.019 |
| 20A | 0.068 |
| 21.1 | 0.019 |
| 21.2 | 0.037 |
| 21.3 | 0.02 |
| 21.4 | 0.03 |
| 21.5 | 0.028 |
| 21.6 | 0.026 |
| 21.7 | 0.031 |
| 21.8 | 0.02 |
| 21.9 | 0.017 |
| 21.10 | 0.018 |
| 21.11 | 0.023 |
| 21.12 | 0.024 |
| 21.13 | 0.032 |
| 21.14 | 0.028 |
| 21.15 | 0.026 |
| 21.16 | 0.022 |
| 21.17 | 0.028 |
| 21.18 | 0.017 |
| 21.19 | 0.023 |
| 21.20 | 0.052 |
| 22 | 0.015 |
| 23 | 0.009 |
| 24.1 | 0.008 |
| 24.2 | 0.006 |
| 24.3 | 0.005 |
| 24.4 | 0.006 |
| 24.5 | 0.005 |
| 24.6 | 0.006 |
| 24.7 | 0.005 |
| 24.8 | 0.007 |
| 24.9 | 0.006 |
| 24.10 | 0.004 |
| 24.11 | 0.015 |
| 24.12 | 0.006 |
| 24.13 | 0.005 |
| 24.14 | 0.008 |
| 25.1 | 0.009 |
| 25.1A | 0.008 |
| 25.2 | 0.013 |
| 25.3 | 0.014 |
| 25.4 | 0.013 |
| 25.5 | 0.009 |
| 25.6 | 0.011 |
| 26 | 0.057 |
| 27.1 | 0.031 |
| 27.2 | 0.036 |
| 27.3 | 0.022 |
| 27.4 | 0.024 |
| 27.5 | 0.023 |
| 27.6 | 0.014 |
| 28.1 | 0.005 |
| 28.2 | 0.006 |
| 28.3 | 0.006 |
| 28.4 | 0.014 |
| 28.5 | 0.004 |
| 29 | 0.017 |
| 30 | 0.033 |
| 31 | 0.021 |
| 32 | 0.019 |
| 33 | 0.057 |
| 34 | 0.098 |
| 34A | 0.132 |
| 35 | 0.021 |
| 36 | 0.04 |
| 37 | 0.031 |
| 38 | 0.044 |
| 39 | 0.012 |
| 40 | 0.008 |
| 41 | 0.009 |
| 42 | 0.018 |
| 43 | 0.01 |
| 44 | 0.027 |
| 45 | 0.024 |
| 46 | 0.013 |
| 47.1 | 0.009 |
| 47.2 | 0.008 |
| 47.3 | 0.006 |
| 48 | 0.017 |
| 49 | 0.014 |
| 50 | 0.008 |
| 51 | 0.008 |
| 52 | 0.01 |
| 53 | 0.008 |
| 54 | 0.008 |
| 55 | 0.033 |
| 56 | 0.006 |
| 57 | 0.006 |
| 58 | 0.006 |
| 59.1 | 0.017 |
| 59.2 | 0.007 |
| 60 | 0.113 |
| 61 | 0.021 |
| 62 | 0.019 |
| 63 | 0.016 |
| 64 | 0.019 |
| 65 | 0.012 |
| 66 | 0.015 |
| 67 | 0.007 |
| 68 | 0.024 |
| 69 | 0.032 |
| 70 | 0.018 |
| 71 | 0.01 |
| 72 | 0.096 |
| 73 | 0.071 |
| 74 | 0.042 |
| 75 | 0.172 |
| 76 | 0.033 |
| 77 | 0.184 |
| 78 | 0.054 |
| 79 | 0.056 |
| 80 | 0.133 |
| 81 | 0.03 |
| 82 | 0.043 |
| 83 | 0.167 |
| 84 | 0.179 |
| 85 | 0.034 |
| 86 | 0.012 |
| 87 | 0.065 |
| 88 | 0.023 |
| 89 | 0.034 |
| 90 | 0.037 |
| 91 | 0.035 |
| 92 | 0.09 |
| 93 | 0.027 |
| 94 | 0.136 |
| 95 | 0.06 |
| 96 | 0.127 |
| 97 | 0.047 |
| 98 | 0.022 |
| 99 | 0.024 |
| 100 | 0.057 |
| 101 | 0.235 |
| 102 | 0.054 |
| 103 | 0.077 |
| 104 | 0.029 |
| 105 | 0.026 |
| 106 | 0.028 |
| 107 | 0.015 |
| 108 | 0.014 |
| 109 | 0.016 |
| 110 | 0.036 |
| 111 | 0.049 |
| 112 | 0.042 |
| 113 | 0.033 |
| 114 | 0.013 |
| 115 | 0.005 |
| 116 | 0.013 |
| 118 | 0.092 |
| 119 | 0.095 |
| 120 | 0.128 |
| 121 | 0.015 |
| 122 | 0.008 |
| 123 | 0.005 |
| 124 | 0.031 |
| 125 | 0.042 |
| 126.1 | 0.009 |
| 126.2 | 0.038 |
| 126.3 | 0.021 |
| 126.4 | 0.009 |
| 126.5 | 0.217 |
| 126.6 | 0.044 |
| 126.7 | 0.047 |
| 126.8 | 0.021 |
| 126.9 | 0.008 |
| 126.10 | 0.008 |
| 126.11 | 0.007 |
| 126.12 | 0.025 |
| 126.13 | 0.106 |
| 126.14 | 0.006 |
| 126.15 | 0.016 |
| 126.16 | 0.03 |
| 126.17 | 0.032 |
| 126.18 | 0.013 |
| 126.19 | 0.015 |
| 127 | 0.007 |
| 128 | 0.006 |
| 129 | 0.003 |
| 130 | 0.003 |
| 131 | 0.003 |
| 132 | 0.003 |
| 133 | 0.003 |
| 134 | 0.005 |
| 135A | 0.011 |
| 135B | 0.008 |
| 137 | 0.003 |
| 138 | 0.003 |
| 139 | 0.012 |
| 140 | 0.003 |
| 141 | 0.001 |
| 142 | 0.002 |
| 143 | 0.002 |
| 144 | 0.005 |
| 145 | 0.059 |
| 146 | 0.140 |
| 147 | 0.014 |
| 148 | 0.017 |
| 149 | 0.007 |
| 150 | 0.005 |
| 151 | 0.013 |
| 152 | 0.025 |
| 153 | 0.000 |
| 154 | 0.004 |
| 155 | 0.120 |
| 156 | 0.004 |
| 157 | 0.001 |
| 158 | 0.015 |
| 159 | 0.016 |
| 160 | 0.014 |
| 161 | 0.011 |
| 162 | 0.002 |
| 163 | 0.016 |
| 164 | 0.015 |
| 165 | 0.042 |
| 166 | 0.017 |
| 167 | 0.049 |
| 169 | 0.005 |
| 170 | 0.067 |
| 171 | 0.017 |
| 172 | 0.034 |
| 173 | 0.010 |
| 174 | 0.063 |
| 175 | 0.014 |
| 176 | 0.022 |
| 177 | 0.005 |
| 178 | 0.015 |
| 179 | 0.008 |
| 180 | 0.025 |
| 181 | 0.021 |
| 182 | 0.001 |
| 183 | 0.007 |
| 184 | 0.007 |
| 185 | 0.170 |
| 186 | 0.036 |
| 187 | 0.043 |
| 188 | 0.120 |
| 189 | 0.095 |
| 190 | 0.002 |
| 191 | 0.004 |
| 192 | 0.530 |
| 193 | 0.025 |
| 194 | 0.015 |
| 195 | 0.033 |
| 196 | 0.013 |
| 197 | 0.013 |
| 198 | 0.026 |
| 199 | 0.110 |
| 200 | 0.032 |
| 201 | 0.088 |
| 202 | 0.096 |
| 203 | 0.009 |
| 204 | 0.019 |
| 205 | 0.007 |
| 206 | 0.000 |
| 207 | 0.019 |
| 208 | 0.026 |
| 209 | 0.030 |
| 210 | 0.036 |
| 211 | 0.019 |
| 212 | 0.019 |
| 213 | 0.014 |
| 214 | 0.018 |
| 215 | 0.009 |
| 216 | 0.002 |
| 217 | 0.010 |
| 218 | 0.007 |
| 219 | 0.009 |
| 220 | 0.005 |
| 221 | 0.010 |
| 222 | 0.003 |
| 223 | 0.006 |
| 224 | 0.003 |
| 225 | 0.130 |
| 226 | 0.033 |
| 227 | 0.048 |
| 228 | 0.024 |
| 229 | 0.020 |
| 230 | 0.005 |
| 231 | 0.020 |
| 232 | 0.023 |
| 233 | 0.009 |
| 234 | 0.031 |
| 235 | 0.013 |
| 236 | 0.049 |
| 237 | 0.008 |
| 238 | 0.008 |
| 239 | 0.110 |
| 240 | 0.003 |
| 241 | 0.036 |
| 242 | 0.740 |
| 243 | 0.210 |
| 244 | 0.005 |
| 245 | 0.004 |
| 246 | 0.004 |
| 247 | 0.003 |
| 248 | 0.006 |
| 249 | 0.005 |
| 250 | 0.007 |
| 251 | 0.009 |
| 252 | 0.007 |
| 253 | 0.004 |
| 254 | 0.005 |
| 255 | 0.005 |
| 256 | 0.005 |
| 257 | 0.010 |
| 258 | 0.005 |
| 259 | 0.004 |
| 260 | 0.011 |
| 261 | 0.023 |
| 262 | 0.039 |
| 263 | 0.007 |
| 264 | 0.012 |
| 265 | 0.006 |
| 266 | 0.029 |
| 267 | 0.027 |
| 268 | 0.023 |
| 269 | 0.016 |
| 270 | 0.043 |
| 271 | 0.190 |
| 272 | 0.270 |
| 273 | 0.210 |
| 274 | 0.014 |
| 275 | 0.009 |
| 276 | 0.015 |
| 277 | 0.016 |
| 278 | 0.008 |
| 279 | 0.008 |

1. A compound of formula (I): 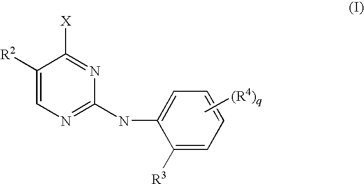
or a pharmaceutically acceptable salt thereof,
wherein:
X is selected from a group of formula Ia and Ib: 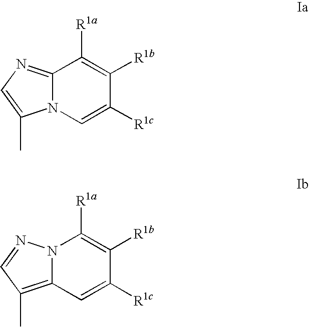
each R^{1a}, R^{1b} and R^{1c}, which may be the same or different, is selected from hydrogen, halogeno, cyano, (C1-C6)alkyl, (C1-C6)alkoxy, amino, (C1-C6)alkylamino and di-[(C1-C6)alkyl]amino, each of which groups within R^{1a}, R^{1b} and R^{1c} may be optionally substituted by one or more substituents independently selected from hydroxy, halogeno, cyano, (C1-C6)alkyl, (C1-C6)alkoxy, amino, (C1-C6)alkylamino, di-[(C1-C6)alkyl]amino, —N(R″)C(O)R′ wherein R′ is selected from hydrogen, (C1-C6)alkyl and (C1-C6)alkoxy and R″ is selected from hydrogen and (C1-C6)alkyl, and a saturated monocyclic 4-, 5-, 6-, 7- or 8-membered ring optionally comprising one or more heteroatoms independently selected from nitrogen, oxygen and sulfur;
R² is selected from halogeno, cyano, trifluoromethyl, cyclopropyl, (C1-C3)alkyl and (C1-C3)alkoxy;
R³ is selected from hydroxy, cyano, halogeno, (C1-C6)alkyl and (C1-C6)alkoxy, each of which groups within R³ may be optionally substituted by one or more substituents independently selected from hydroxy, halogeno, cyano and (C1-C6)alkoxy;
q represents 0, 1, 2, 3 or 4;
each R⁴, which may be the same or different, is selected from hydroxy, cyano, halogeno, formyl, carboxy, (C1-C6)alkyl, (C2-C6)alkenyl, (C2-C6)alkynyl, (C1-C6)alkoxy, (C2-C6)alkanoyl, (C1-C6)alkoxycarbonyl, (C3-C8)cycloalkyl, (C3-C8)cycloalkylcarbonyl, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (C1-C6)alkoxyamino, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, sulfamoyl, (C1-C6)alkylsulfamoyl, di-[(C1-C6)alkyl]sulfamoyl, —S(O)ₘR′ wherein R′ is as defined above and m represents 0, 1 or 2, —N(R″)C(O)R′ wherein R′ and R″ are each as defined above, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,
or two R⁴ groups on adjacent carbon atoms of the phenyl ring, together with the carbon atoms to which they are attached, form a saturated or unsaturated monocyclic 5- or 6-membered heterocyclic ring comprising at least one ring heteroatom selected from nitrogen, oxygen and sulfur,
each of which groups or rings within R⁴ may be optionally substituted by one or more substituents independently selected from hydroxy, halogeno, cyano, formyl, carboxy, (C1-C6)alkyl, (C3-C8)cycloalkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, (C1-C6)alkylthio, sulfamoyl, (C1-C6)alkylsulfamoyl, di-[(C1-C6)alkyl]sulfamoyl, —S(O)ₘR′ wherein R′ and m are each as defined above, —N(R″)C(O)R′ wherein R′ and R″ are each as defined above, and —X-Q wherein X and Q are each as defined above, any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ and m are each as defined above, —N(R″)C(O)R′ wherein R′ and R″ are each as defined above, and —X-Q wherein X and Q are each as defined above;
and wherein any saturated monocyclic ring optionally bears 1 or 2 oxo or thioxo substituents.

2. A compound according to claim 1, wherein X is a group of formula Ia as defined in claim 1.

3. A compound according to claim 1, wherein X is a group of formula Ib as defined in claim 1.

4. A compound according to claim 1, wherein each R^{1a}, R^{1b} and R^{1c}, which may be the same or different, is selected from hydrogen, halogeno, cyano, (C1-C3)alkyl, (C1-C3)alkoxy, amino, (C1-C3)alkylamino and di-[(C1-C3)alkyl]amino, each of which groups within R^{1a}, R^{1b} and R^{1c} may be optionally substituted by one or more substituents independently selected from hydroxy and halogeno.

5. A compound according to claim 4, wherein each R^{1a}, R^{1b} and R^{1c}, which may be the same or different, is selected from hydrogen, halogeno, cyano, methyl, ethyl, methoxy and dimethylamino, each of which groups within R^{1a}, R^{1b} and R^{1c} may be optionally substituted by one or more substituents independently selected from hydroxy and halogeno.

6. A compound according to claim 4 wherein R^{1a}, R^{1b} and R^{1c} all represent hydrogen.

7. A compound according to claim 1, wherein R² is selected from halogeno, cyano, trifluoromethyl, (C1-C3)alkyl and (C1-C3)alkoxy.

8. A compound according to claim 7, wherein R² is halogeno.

9. A compound according to claim 7, wherein R² is methyl, fluoro or chloro.

10. A compound according to claim 1, wherein R³ is selected from halogeno, (C1-C6)alkyl and (C1-C6)alkoxy, each of which groups within R³ may be optionally substituted by one or more substituents independently selected from hydroxy, halogeno, cyano and (C1-C6)alkoxy.

11. A compound according to claim 10, wherein R³ is (C1-C3)alkoxy.

12. A compound according to claim 11, wherein R³ is methoxy.

13. A compound according to claim 1, wherein q is 1 or 2.

14. A compound according to claim 13, wherein each R⁴, which may be the same or different, may be selected from hydroxy, cyano, halogeno, formyl, carboxy, (C1-C6)alkyl, (C2-C6)alkenyl, (C2-C6)alkynyl, (C1-C6)alkoxy, (C2-C6)alkanoyl, (C1-C6)alkoxycarbonyl, (C3-C8)cycloalkyl, (C3-C8)cycloalkylcarbonyl, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,
each of which groups or rings within R⁴ may be optionally substituted by one or more substituents independently selected from hydroxy, halogeno, cyano, carboxy, (C1-C6)alkyl, (C3-C8)cycloalkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, (C1-C6)alkylthio, sulfamoyl, (C1-C6)alkylsulfamoyl, di-[(C1-C6)alkyl]sulfamoyl, —S(O)ₘR′ wherein R′ is selected from hydrogen and (C1-C6)alkyl and m represents 0, 1 or 2, —N(R″)C(O)R′ wherein R′ is selected from hydrogen and (C1-C6)alkyl and R″ is selected from hydrogen and (C1-C6)alkyl, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur, any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ is selected from hydrogen and (C1-C6)alkyl and m represents 0, 1 or 2, —N(R″)C(O)R′ wherein R′ is selected from hydrogen and (C1-C6)alkyl and R″ is selected from hydrogen and (C1-C6)alkyl, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur;
and wherein any saturated monocyclic ring optionally bears 1 or 2 oxo substituents.

15. A compound according to claim 13, wherein each R⁴, which may be the same or different, may be selected from (C1-C6)alkyl, (C1-C6)alkoxy and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur,
each of which groups or rings within R⁴ may be optionally substituted by one or more substituents independently selected from hydroxy, carboxy, (C1-C6)alkyl, (C3-C8)cycloalkylcarbonyl, (C1-C6)alkoxy, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (2-6C)alkanoyl, (C1-C6)alkoxycarbonyl, carbamoyl, (C1-C6)alkylcarbamoyl, di-[(C1-C6)alkyl]carbamoyl, carbamoyl(C1-C6)alkyl, (C1-C6)alkylcarbamoyl(C1-C6)alkyl, di-[(C1-C6)alkyl]carbamoyl(C1-C6)alkyl, —S(O)ₘR′ wherein R′ is selected from hydrogen and (C1-C6)alkyl and m represents 0, 1 or 2, —N(R″)C(O)R′ wherein R′ is selected from hydrogen and (C1-C6)alkyl and R″ is selected from hydrogen and (C1-C6)alkyl, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur, any of which substituents may be optionally substituted by one or more further substituents independently selected from (C1-C4)alkyl, (C1-C4)alkoxy, hydroxy, halogeno, cyano, hydroxy(C1-C4)alkyl, amino, (C1-C6)alkylamino, di-[(C1-C6)alkyl]amino, carbamoyl, (C1-C6)alkylcarbamoyl, di-[(C1-C6)alkyl]carbamoyl, —S(O)ₘR′ wherein R′ is selected from hydrogen and (C1-C6)alkyl and m represents 0, 1 or 2, —N(R″)C(O)R′ wherein R′ is selected from hydrogen and (C1-C6)alkyl and R″ is selected from hydrogen and (C1-C6)alkyl, and —X-Q wherein X is selected from a direct bond, —O—, —C(O)—, (C1-C4)alkyl and (C1-C4)alkoxy and Q represents a saturated 4-, 5-, 6-, 7-, 8-, 9- or 10-membered heterocyclic ring comprising at least one ring heteroatom selected from nitrogen, oxygen and sulfur;
and wherein any saturated monocyclic ring optionally bears 1 or 2 oxo substituents.

16. A compound according to claim 1, where in the compound of formula I is a compound of Formula (IAi) 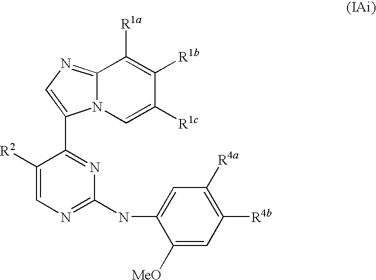
or a compound of formula (IBi) 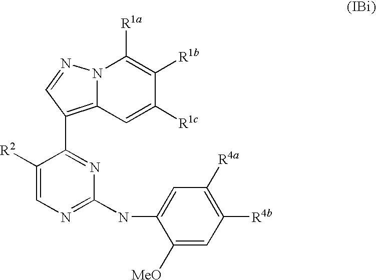
wherein
R^{1a}, R^{1b}, R^{1c} and R² are as defined above; and
either
R^{4a} & R^{4b} groups on adjacent carbon atoms of the phenyl ring, together with the carbon atoms to which they are attached, form a saturated or unsaturated monocyclic 5-membered heterocyclic ring comprising one ring nitrogen which is fused to the phenyl ring in such a way as to form an indolinyl ring and wherein the heterocyclic ring of the indolinyl ring is substituted on the ring nitrogen by (2-3C)alkanoyl which may be optionally substituted by one or more further substituents independently selected from (C1-C3)alkyl, (C1-C3)alkoxy, hydroxy, halogeno, hydroxy(C1-C3)alkyl, amino, (C1-C3)alkylamino and di-[(C1-C3)alkyl]amino;
or
one of R^{4a} and R^{4b} is hydrogen and the remaining R^{4a} or R^{4b} is —X-Q wherein X is a direct bond and Q represents a piperazin-1-yl which may be optionally substituted by one or more substituents independently selected from (C1-C6)alkyl and (2-6C)alkanoyl, any of which substituents may be optionally substituted by one or more further substituents independently selected from (C1-C4)alkyl, (C1-C4)alkoxy, (C1-C4)alkylamino, di-[(C1-C4)alkyl]amino, hydroxy and hydroxy(C1-C4)alkyl,
and wherein any saturated monocyclic ring optionally bears 1 or 2 oxo substituents.

17. A compound according to claim 16 wherein R^{1a}, R^{1b} and R^{1c} all represent hydrogen; R² is methyl, fluoro or chloro; and R³ is methoxy.

18. A compound according to claim 1 selected from
1-[4-[4-[(5-chloro-4-imidazo[1,2-a]pyridin-3-yl-pyrimidin-2-yl)amino]-3-methoxy-phenyl]piperazin-1-yl]ethanone;
5-chloro-4-imidazo[1,2-a]pyridin-3-yl-N-(2-methoxy-4-piperazin-1-yl-phenyl)pyrimidin-2-amine;
[4-[(5-chloro-4-imidazo[1,2-a]pyridin-3-yl-pyrimidin-2-yl)amino]-3-methoxy-phenyl]-morpholino-methanone;
[1-[4-[(5-chloro-4-imidazo[1,2-a]pyridin-3-yl-pyrimidin-2-yl)amino]-3-methoxy-phenyl]pyrrolidin-2-yl]-morpholino-methanone;
2-[4-[4-[(5-chloro-4-imidazo[1,2-a]pyridin-3-yl-pyrimidin-2-yl)amino]-3-methoxy-phenyl]piperazin-1-yl]-1-morpholino-ethanone;
[3-[(5-chloro-4-imidazo[1,2-a]pyridin-3-yl-pyrimidin-2-yl)amino]-4-methoxy-phenyl]-morpholino-methanone;
(3S)-1-[3-[(5-chloro-4-imidazo[1,2-a]pyridin-3-yl-pyrimidin-2-yl)amino]-4-methoxy-phenyl]pyrrolidin-3-ol;
5-chloro-N-[4-(3,5-dimethylpiperazin-1-yl)-2-methoxy-phenyl]-4-imidazo[1,2-a]pyridin-3-yl-pyrimidin-2-amine;
4-[4-[(5-chloro-4-imidazo[1,2-a]pyridin-3-yl-pyrimidin-2-yl)amino]-3-methoxy-phenyl]-N,N-dimethyl-piperazine-1-sulfonamide;
5-chloro-4-imidazo[1,2-a]pyridin-3-yl-N-(2-methoxy-5-pyrrolidin-1-yl-phenyl)pyrimidin-2-amine;
1-[4-[3-[(5-chloro-4-imidazo[1,2-a]pyridin-3-yl-pyrimidin-2-yl)amino]-4-methoxy-phenyl]piperazin-1-yl]ethanone;
5-chloro-4-imidazo[1,2-a]pyridin-3-yl-N-(2-methoxy-5-piperazin-1-yl-phenyl)pyrimidin-2-amine;
5-chloro-N-[4-[(3R)-3-(dimethylamino)pyrrolidin-1-yl]-2-methoxy-phenyl]-4-imidazo[1,2-a]pyridin-3-yl-pyrimidin-2-amine;
5-chloro-4-imidazo[1,2-a]pyridin-3-yl-N-[2-methoxy-4-[2-[4-(2-methoxyethyl)piperazin-1-yl]ethoxy]phenyl]pyrimidin-2-amine;
5-chloro-N-[5-[(3R)-3-(dimethylamino)pyrrolidin-1-yl]-2-methoxy-phenyl]-4-imidazo[1,2-a]pyridin-3-yl-pyrimidin-2-amine;
5-chloro-N-[5-[(3S)-3-(dimethylamino)pyrrolidin-1-yl]-2-methyl-phenyl]-4-imidazo[1,2-a]pyridin-3-yl-pyrimidin-2-amine;
5-chloro-N-[4-(1,1-dioxo-1,4-thiazinan-4-yl)-2-methoxy-phenyl]-4-imidazo[1,2-a]pyridin-3-yl-pyrimidin-2-amine;
5-chloro-N-[4-[(3S)-3-(dimethylamino)pyrrolidin-1-yl]-2-methoxy-phenyl]-4-imidazo[1,2-a]pyridin-3-yl-pyrimidin-2-amine;
5-chloro-N-[5-[(3S)-3-(dimethylamino)pyrrolidin-1-yl]-2-methoxy-phenyl]-4-imidazo[1,2-a]pyridin-3-yl-pyrimidin-2-amine;
(3R)-1-[3-[(5-chloro-4-imidazo[1,2-a]pyridin-3-yl-pyrimidin-2-yl)amino]-4-methoxy-phenyl]pyrrolidin-3-ol;
5-chloro-4-imidazo[1,2-a]pyridin-3-yl-N-(2-methoxy-5-morpholino-phenyl)pyrimidin-2-amine;
5-chloro-4-imidazo[1,2-a]pyridin-3-yl-N-[2-methoxy-5-(4-methoxy-1-piperidyl)phenyl]pyrimidin-2-amine;
1-[4-[4-[(5-chloro-4-imidazo[1,2-a]pyridin-3-yl-pyrimidin-2-yl)amino]-3-methoxy-phenyl]-1-piperidyl]ethanone;
5-chloro-4-imidazo[1,2-a]pyridin-3-yl-N-[2-methoxy-4-(4-methylsulfonylpiperazin-1-yl)phenyl]pyrimidin-2-amine;
N-[(3R)-1-[4-[(5-chloro-4-imidazo[1,2-a]pyridin-3-yl-pyrimidin-2-yl)amino]-3-methoxy-phenyl]pyrrolidin-3-yl]acetamide;
4-[4-[(5-chloro-4-imidazo[1,2-a]pyridin-3-yl-pyrimidin-2-yl)amino]-3-methoxy-phenyl]pyrrolidin-2-one;
1-[2-[4-[(5-chloro-4-imidazo[1,2-a]pyridin-3-yl-pyrimidin-2-yl)amino]-3-methoxy-phenyl]-1-piperidyl]ethanone;
1-[4-[4-[(5-chloro-4-imidazo[1,2-a]pyridin-3-yl-pyrimidin-2-yl)amino]-3-fluoro-5-methoxy-phenyl]piperazin-1-yl]ethanone;
4-[4-[(5-chloro-4-imidazo[1,2-a]pyridin-3-yl-pyrimidin-2-yl)amino]-3-methoxy-phenyl]-N-methyl-pyrrolidine-2-carboxamide;
1-[4-[4-[(5-chloro-4-imidazo[1,2-a]pyridin-3-yl-pyrimidin-2-yl)amino]-3-methoxy-phenyl]-1,4-diazepan-1-yl]ethanone;
1-[3-[4-[(5-chloro-4-imidazo[1,2-a]pyridin-3-yl-pyrimidin-2-yl)amino]-3-methoxy-phenyl]-1-piperidyl]ethanone;
1-[4-[4-[(5-chloro-4-imidazo[1,2-a]pyridin-3-yl-pyrimidin-2-yl)amino]-3-methyl-phenyl]piperazin-1-yl]ethanone;
1-[4-[4-[(5-chloro-4-imidazo[1,2-a]pyridin-3-yl-pyrimidin-2-yl)amino]-3-methoxy-phenyl]-2,6-dimethyl-piperazin-1-yl]ethanone;
1-[4-[4-[(5-chloro-4-imidazo[1,2-a]pyridin-3-yl-pyrimidin-2-yl)amino]-2,3-dimethyl-phenyl]piperazin-1-yl]ethanone;
1-[4-[4-[(5-chloro-4-imidazo[1,2-a]pyridin-3-yl-pyrimidin-2-yl)amino]-2-fluoro-5-methyl-phenyl]piperazin-1-yl]ethanone;
1-[4-[4-[(5-chloro-4-imidazo[1,2-a]pyridin-3-yl-pyrimidin-2-yl)amino]-3-isopropoxy-phenyl]piperazin-1-yl]ethanone;
1-[(2S,5R)-4-[4-[(5-chloro-4-imidazo[1,2-a]pyridin-3-yl-pyrimidin-2-yl)amino]-3-methoxy-phenyl]-2,5-dimethyl-piperazin-1-yl]ethanone;
1-[4-[4-[(5-chloro-4-imidazo[1,2-a]pyridin-3-yl-pyrimidin-2-yl)amino]-3-methoxy-phenyl]-2-methyl-piperazin-1-yl]ethanone;
1-[4-[3-[(5-chloro-4-imidazo[1,2-a]pyridin-3-yl-pyrimidin-2-yl)amino]-2-methyl-phenyl]piperazin-1-yl]ethanone;
1-[4-[4-[(5-chloro-4-imidazo[1,2-a]pyridin-3-yl-pyrimidin-2-yl)amino]-3-methoxy-phenyl]-2-(hydroxymethyl)piperazin-1-yl]ethanone;
2-[4-[(5-chloro-4-imidazo[1,2-a]pyridin-3-yl-pyrimidin-2-yl)amino]-3-methoxy-phenyl]-1-morpholino-propan-1-one;
1-[(6S)-4-[4-[(5-chloro-4-imidazo[1,2-a]pyridin-3-yl-pyrimidin-2-yl)amino]-3-methoxy-phenyl]-6-hydroxy-1,4-diazepan-1-yl]ethanone;
2-[4-[(5-chloro-4-imidazo[1,2-a]pyridin-3-yl-pyrimidin-2-yl)amino]-3-methoxy-phenyl]-1-[4-(2-hydroxyethyl)piperazin-1-yl]propan-1-one;
2-[4-[(5-chloro-4-imidazo[1,2-a]pyridin-3-yl-pyrimidin-2-yl)amino]-3-methoxy-phenyl]-1-piperazin-1-yl-propan-1-one;
5-chloro-4-imidazo[1,2-a]pyridin-3-yl-N-(2-methoxy-4-morpholino-phenyl)pyrimidin-2-amine;
5-chloro-4-imidazo[1,2-a]pyridin-3-yl-N-(2-methoxy-4-pyrrolidin-1-yl-phenyl)pyrimidin-2-amine;
2-[4-[4-[(5-chloro-4-imidazo[1,2-a]pyridin-3-yl-pyrimidin-2-yl)amino]-3-methoxy-phenyl]piperazin-1-yl]ethanol;
5-chloro-4-imidazo[1,2-a]pyridin-3-yl-N-[2-methoxy-4-[4-(2-methoxyethyl)piperazin-1-yl]phenyl]pyrimidin-2-amine;
(3S)-1-[4-[(5-chloro-4-imidazo[1,2-a]pyridin-3-yl-pyrimidin-2-yl)amino]-3-methoxy-phenyl]pyrrolidin-3-ol;
5-chloro-4-imidazo[1,2-a]pyridin-3-yl-N-[2-methoxy-4-(1,4-oxazepan-4-yl)phenyl]pyrimidin-2-amine;
(3R)-1-[4-[(5-chloro-4-imidazo[1,2-a]pyridin-3-yl-pyrimidin-2-yl)amino]-3-methoxy-phenyl]pyrrolidin-3-ol;
5-chloro-4-imidazo[1,2-a]pyridin-3-yl-N-[2-methoxy-4-(4-piperidyl)phenyl]pyrimidin-2-amine;
5-chloro-4-imidazo[1,2-a]pyridin-3-yl-N-[2-methoxy-4-(4-piperidyloxy)phenyl]pyrimidin-2-amine;
5-chloro-4-imidazo[1,2-a]pyridin-3-yl-N-[2-methoxy-4-[[(2R)-pyrrolidin-2-yl]methoxy]phenyl]pyrimidin-2-amine;
5-chloro-4-imidazo[1,2-a]pyridin-3-yl-N-[2-methoxy-4-[[(2S)-pyrrolidin-2-yl]methoxy]phenyl]pyrimidin-2-amine;
5-chloro-4-imidazo[1,2-a]pyridin-3-yl-N-[2-methoxy-5-[[(2S)-pyrrolidin-2-yl]methoxy]phenyl]pyrimidin-2-amine;
5-chloro-4-imidazo[1,2-a]pyridin-3-yl-N-[2-methoxy-5-[[(2R)-pyrrolidin-2-yl]methoxy]phenyl]pyrimidin-2-amine;
5-chloro-4-imidazo[1,2-a]pyridin-3-yl-N-[2-methoxy-4-[(3R)-pyrrolidin-3-yl]oxy-phenyl]pyrimidin-2-amine;
5-chloro-4-imidazo[1,2-a]pyridin-3-yl-N-[2-methoxy-4-[(3S)-pyrrolidin-3-yl]oxy-phenyl]pyrimidin-2-amine;
1-[4-[4-[(5-chloro-4-imidazo[1,2-a]pyridin-3-yl-pyrimidin-2-yl)amino]-3-methoxy-phenoxy]-1-piperidyl]ethanone;
5-chloro-4-imidazo[1,2-a]pyridin-3-yl-N-[2-methoxy-4-[(1-methylsulfonyl-4-piperidyl)oxy]phenyl]pyrimidin-2-amine;
(2S)-1-[4-[4-[(5-chloro-4-imidazo[1,2-a]pyridin-3-yl-pyrimidin-2-yl)amino]-3-methoxy-phenoxy]-1-piperidyl]-2-hydroxy-propan-1-one;
1-[4-[4-[(5-chloro-4-imidazo[1,2-a]pyridin-3-yl-pyrimidin-2-yl)amino]-3-methoxy-phenoxy]-1-piperidyl]-2-hydroxy-propan-1-one;
1-[(3R)-3-[4-[(5-chloro-4-imidazo[1,2-a]pyridin-3-yl-pyrimidin-2-yl)amino]-3-methoxy-phenoxy]pyrrolidin-1-yl]ethanone;
5-chloro-4-imidazo[1,2-a]pyridin-3-yl-N-[2-methoxy-4-[(3R)-1-methylsulfonylpyrrolidin-3-yl]oxy-phenyl]pyrimidin-2-amine;
1-[(3R)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one;
(2S)-1-[(3R)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one;
1-[(3S)-3-[4-[(5-chloro-4-imidazo[1,2-a]pyridin-3-yl-pyrimidin-2-yl)amino]-3-methoxy-phenoxy]pyrrolidin-1-yl]ethanone;
5-chloro-4-imidazo[1,2-a]pyridin-3-yl-N-[2-methoxy-4-[(3S)-1-methylsulfonylpyrrolidin-3-yl]oxy-phenyl]pyrimidin-2-amine;
(2S)-1-[(3S)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one;
1-[(3S)-3-[4-[(5-chloro-4-imidazo[1,2-a]pyridin-3-yl-pyrimidin-2-yl)amino]-3-methoxy-phenoxy]pyrrolidin-1-yl]-2-hydroxy-propan-1-one;
1-[(2S)-2-[[4-[(5-chloro-4-imidazo[1,2-a]pyridin-3-yl-pyrimidin-2-yl)amino]-3-methoxy-phenoxy]methyl]pyrrolidin-1-yl]ethanone;
1-[(2R)-2-[[3-[(5-chloro-4-imidazo[1,2-a]pyridin-3-yl-pyrimidin-2-yl)amino]-4-methoxy-phenoxy]methyl]pyrrolidin-1-yl]ethanone;
4-[4-[(5-chloro-4-imidazo[1,2-a]pyridin-3-yl-pyrimidin-2-yl)amino]-3-methoxy-phenyl]piperazin-2-one;
4-[4-[(5-chloro-4-imidazo[1,2-a]pyridin-3-yl-pyrimidin-2-yl)amino]-3-methoxy-phenyl]-N,N-dimethyl-piperazine-2-carboxamide;
(3S)-1-[[4-[(5-chloro-4-imidazo[1,2-a]pyridin-3-yl-pyrimidin-2-yl)amino]-3-methoxy-phenyl]methyl]pyrrolidin-3-ol;
2-[[4-[(5-chloro-4-imidazo[1,2-a]pyridin-3-yl-pyrimidin-2-yl)amino]-3-methoxy-phenyl]methylamino]ethanol;
2-[[4-[(5-chloro-4-imidazo[1,2-a]pyridin-3-yl-pyrimidin-2-yl)amino]-3-methoxy-phenyl]methyl-methyl-amino]ethanol;
3-[[4-[(5-chloro-4-imidazo[1,2-a]pyridin-3-yl-pyrimidin-2-yl)amino]-3-methoxy-phenyl]methylamino]propan-1-ol;
5-chloro-4-imidazo[1,2-a]pyridin-3-yl-N-[2-methoxy-4-[(4-methylpiperazin-1-yl)methyl]phenyl]pyrimidin-2-amine;
1-[[4-[(5-chloro-4-imidazo[1,2-a]pyridin-3-yl-pyrimidin-2-yl)amino]-3-methoxy-phenyl]methyl]piperidin-4-ol;
1-[4-[[4-[(5-chloro-4-imidazo[1,2-a]pyridin-3-yl-pyrimidin-2-yl)amino]-3-methoxy-phenyl]methyl]piperazin-1-yl]ethanone;
1-[[4-[(5-chloro-4-imidazo[1,2-a]pyridin-3-yl-pyrimidin-2-yl)amino]-3-methoxy-phenyl]methyl]piperidine-4-carboxamide;
2-[4-[[4-[(5-chloro-4-imidazo[1,2-a]pyridin-3-yl-pyrimidin-2-yl)amino]-3-methoxy-phenyl]methyl]piperazin-1-yl]ethanol;
5-chloro-4-imidazo[1,2-a]pyridin-3-yl-N-[2-methoxy-4-[(3-methoxypropylamino)methyl]phenyl]pyrimidin-2-amine;
N-[4-(azetidin-1-ylmethyl)-2-methoxy-phenyl]-5-chloro-4-imidazo[1,2-a]pyridin-3-yl-pyrimidin-2-amine;
5-chloro-4-imidazo[1,2-a]pyridin-3-yl-N-[2-methoxy-4-[(4-methyl-1,4-diazepan-1-yl)methyl]phenyl]pyrimidin-2-amine;
(3R)-1-[[4-[(5-chloro-4-imidazo[1,2-a]pyridin-3-yl-pyrimidin-2-yl)amino]-3-methoxy-phenyl]methyl]piperidin-3-ol;
5-chloro-4-imidazo[1,2-a]pyridin-3-yl-N-[2-methoxy-4-[[2-methoxyethyl(methyl)amino]methyl]phenyl]pyrimidin-2-amine;
5-chloro-4-imidazo[1,2-a]pyridin-3-yl-N-[2-methoxy-4-(1,4-oxazepan-4-ylmethyl)phenyl]pyrimidin-2-amine;
(2R)-2-[[4-[(5-chloro-4-imidazo[1,2-a]pyridin-3-yl-pyrimidin-2-yl)amino]-3-methoxy-phenyl]methylamino]propan-1-ol;
1-[[4-[(5-chloro-4-imidazo[1,2-a]pyridin-3-yl-pyrimidin-2-yl)amino]-3-methoxy-phenyl]methyl]azetidin-3-ol;
(3S)-1-[[4-[(5-chloro-4-imidazo[1,2-a]pyridin-3-yl-pyrimidin-2-yl)amino]-3-methoxy-phenyl]methyl]piperidin-3-ol;
(3R)-1-[[4-[(5-chloro-4-imidazo[1,2-a]pyridin-3-yl-pyrimidin-2-yl)amino]-3-methoxy-phenyl]methyl]pyrrolidin-3-ol;
(2S)-2-[[4-[(5-chloro-4-imidazo[1,2-a]pyridin-3-yl-pyrimidin-2-yl)amino]-3-methoxy-phenyl]methylamino]propan-1-ol;
5-chloro-4-imidazo[1,2-a]pyridin-3-yl-N-[2-methoxy-4-(morpholinomethyl)phenyl]pyrimidin-2-amine;
[4-[4-[(5-chloro-4-imidazo[1,2-a]pyridin-3-yl-pyrimidin-2-yl)amino]-3-methoxy-phenyl]piperazin-1-yl]-(1-hydroxycyclopropyl)methanone;
[4-[4-[(5-chloro-4-imidazo[1,2-a]pyridin-3-yl-pyrimidin-2-yl)amino]-3-methoxy-phenyl]piperazin-1-yl]-[(2S)-pyrrolidin-2-yl]methanone;
[4-[4-[(5-chloro-4-imidazo[1,2-a]pyridin-3-yl-pyrimidin-2-yl)amino]-3-methoxy-phenyl]piperazin-1-yl]-[(2R)-2-piperidyl]methanone;
[4-[4-[(5-chloro-4-imidazo[1,2-a]pyridin-3-yl-pyrimidin-2-yl)amino]-3-methoxy-phenyl]piperazin-1-yl]-[(2S)-2-piperidyl]methanone;
2-amino-1-[4-[4-[(5-chloro-4-imidazo[1,2-a]pyridin-3-yl-pyrimidin-2-yl)amino]-3-methoxy-phenyl]piperazin-1-yl]ethanone;
(2S)-2-amino-1-[4-[4-[(5-chloro-4-imidazo[1,2-a]pyridin-3-yl-pyrimidin-2-yl)amino]-3-methoxy-phenyl]piperazin-1-yl]propan-1-one;
(2S)-2-amino-1-[4-[4-[(5-chloro-4-imidazo[1,2-a]pyridin-3-yl-pyrimidin-2-yl)amino]-3-methoxy-phenyl]piperazin-1-yl]-3-methyl-butan-1-one;
(2S)-2-amino-1-[4-[4-[(5-chloro-4-imidazo[1,2-a]pyridin-3-yl-pyrimidin-2-yl)amino]-3-methoxy-phenyl]piperazin-1-yl]-3-hydroxy-propan-1-one;
(3S)-3-amino-4-[4-[4-[(5-chloro-4-imidazo[1,2-a]pyridin-3-yl-pyrimidin-2-yl)amino]-3-methoxy-phenyl]piperazin-1-yl]-4-oxo-butanamide;
[4-[4-[(5-chloro-4-imidazo[1,2-a]pyridin-3-yl-pyrimidin-2-yl)amino]-3-methoxy-phenyl]piperazin-1-yl]-[(2S,4R)-4-hydroxypyrrolidin-2-yl]methanone;
1-[4-[4-[(5-chloro-4-imidazo[1,2-a]pyridin-3-yl-pyrimidin-2-yl)amino]-3-methoxy-phenyl]piperazin-1-yl]-2-(methylamino)ethanone;
1-[4-[4-[(5-chloro-4-imidazo[1,2-a]pyridin-3-yl-pyrimidin-2-yl)amino]-3-methoxy-phenyl]piperazin-1-yl]-2-(ethylamino)ethanone;
(2R)-2-amino-1-[4-[4-[(5-chloro-4-imidazo[1,2-a]pyridin-3-yl-pyrimidin-2-yl)amino]-3-methoxy-phenyl]piperazin-1-yl]-3-hydroxy-propan-1-one;
(2S)-2-amino-1-[4-[4-[(5-chloro-4-imidazo[1,2-a]pyridin-3-yl-pyrimidin-2-yl)amino]-3-methoxy-phenyl]piperazin-1-yl]-4-hydroxy-butan-1-one;
(2R)-2-amino-1-[4-[4-[(5-chloro-4-imidazo[1,2-a]pyridin-3-yl-pyrimidin-2-yl)amino]-3-methoxy-phenyl]piperazin-1-yl]propan-1-one;
5-[4-[4-[(5-chloro-4-imidazo[1,2-a]pyridin-3-yl-pyrimidin-2-yl)amino]-3-methoxy-phenyl]piperazine-1-carbonyl]pyrrolidin-2-one;
(5S)-5-[4-[4-[(5-chloro-4-imidazo[1,2-a]pyridin-3-yl-pyrimidin-2-yl)amino]-3-methoxy-phenyl]piperazine-1-carbonyl]pyrrolidin-2-one;
N-[2-[4-[4-[(5-chloro-4-imidazo[1,2-a]pyridin-3-yl-pyrimidin-2-yl)amino]-3-methoxy-phenyl]piperazin-1-yl]-2-oxo-ethyl]acetamide;
(2S)-1-[4-[4-[(5-chloro-4-imidazo[1,2-a]pyridin-3-yl-pyrimidin-2-yl)amino]-3-methoxy-phenyl]piperazin-1-yl]-2-methoxy-propan-1-one;
(2R)-1-[4-[4-[(5-chloro-4-imidazo[1,2-a]pyridin-3-yl-pyrimidin-2-yl)amino]-3-methoxy-phenyl]piperazin-1-yl]-2-hydroxy-propan-1-one;
(2R)-1-[4-[4-[(5-chloro-4-imidazo[1,2-a]pyridin-3-yl-pyrimidin-2-yl)amino]-3-methoxy-phenyl]piperazin-1-yl]-2-methoxy-propan-1-one;
1-[4-[4-[(5-chloro-4-imidazo[1,2-a]pyridin-3-yl-pyrimidin-2-yl)amino]-3-methoxy-phenyl]piperazin-1-yl]-2-hydroxy-2-methyl-propan-1-one;
N-[4-(azetidin-3-yloxy)-2-methoxy-phenyl]-5-chloro-4-imidazo[1,2-a]pyridin-3-yl-pyrimidin-2-amine;
(2S)-1-[3-[4-[(5-chloro-4-imidazo[1,2-a]pyridin-3-yl-pyrimidin-2-yl)amino]-3-methoxy-phenoxy]azetidin-1-yl]-2-hydroxy-propan-1-one;
(2S)-1-[3-[4-[(5-chloro-4-imidazo[1,2-a]pyridin-3-yl-pyrimidin-2-yl)amino]-3-methoxy-phenoxy]azetidin-1-yl]-2-methoxy-propan-1-one;
1-[3-[4-[(5-chloro-4-imidazo[1,2-a]pyridin-3-yl-pyrimidin-2-yl)amino]-3-methoxy-phenoxy]azetidin-1-yl]-2-hydroxy-ethanone;
(2R)-1-[3-[4-[(5-chloro-4-imidazo[1,2-a]pyridin-3-yl-pyrimidin-2-yl)amino]-3-methoxy-phenoxy]azetidin-1-yl]-2-methoxy-propan-1-one;
[3-[4-[(5-chloro-4-imidazo[1,2-a]pyridin-3-yl-pyrimidin-2-yl)amino]-3-methoxy-phenoxy]azetidin-1-yl]-(1-hydroxycyclopropyl)methanone;
(2R)-1-[3-[4-[(5-chloro-4-imidazo[1,2-a]pyridin-3-yl-pyrimidin-2-yl)amino]-3-methoxy-phenoxy]azetidin-1-yl]-2-hydroxy-propan-1-one;
(2R)-1-[4-[4-[(5-chloro-4-imidazo[1,2-a]pyridin-3-yl-pyrimidin-2-yl)amino]-3-methoxy-phenyl]-1-piperidyl]-2-methoxy-propan-1-one;
(2R)-1-[4-[4-[(5-chloro-4-imidazo[1,2-a]pyridin-3-yl-pyrimidin-2-yl)amino]-3-methoxy-phenyl]-1-piperidyl]-2-hydroxy-propan-1-one;
(2S)-1-[4-[4-[(5-chloro-4-imidazo[1,2-a]pyridin-3-yl-pyrimidin-2-yl)amino]-3-methoxy-phenyl]-1-piperidyl]-2-methoxy-propan-1-one;
(2S)-1-[4-[4-[(5-chloro-4-imidazo[1,2-a]pyridin-3-yl-pyrimidin-2-yl)amino]-3-methoxy-phenyl]-1-piperidyl]-2-hydroxy-propan-1-one;
1-[4-[4-[(5-chloro-4-imidazo[1,2-a]pyridin-3-yl-pyrimidin-2-yl)amino]-3-methoxy-phenyl]-1-piperidyl]-2-hydroxy-ethanone;
[4-[(5-chloro-4-imidazo[1,2-a]pyridin-3-yl-pyrimidin-2-yl)amino]-3-methoxy-phenyl]-[(1S,4S)-3,6-diazabicyclo[2.2.1]heptan-6-yl]methanone;
[4-[(5-chloro-4-imidazo[1,2-a]pyridin-3-yl-pyrimidin-2-yl)amino]-3-methoxy-phenyl]-piperazin-1-yl-methanone;
[4-[(5-chloro-4-imidazo[1,2-a]pyridin-3-yl-pyrimidin-2-yl)amino]-3-methoxy-phenyl]-[(1S,4S)-5-methyl-2,5-diazabicyclo[2.2.1]heptan-2-yl]methanone;
[4-[(5-chloro-4-imidazo[1,2-a]pyridin-3-yl-pyrimidin-2-yl)amino]-3-methoxy-phenyl]-[4-(2-hydroxyethyl)piperazin-1-yl]methanone;
4-[(5-chloro-4-imidazo[1,2-a]pyridin-3-yl-pyrimidin-2-yl)amino]-N-(2-hydroxyethyl)-3-methoxy-N-methyl-benzamide;
2-[4-[(5-chloro-4-imidazo[1,2-a]pyridin-3-yl-pyrimidin-2-yl)amino]-3-methoxy-phenyl]-1-piperazin-1-yl-ethanone;
2-[4-[(5-chloro-4-imidazo[1,2-a]pyridin-3-yl-pyrimidin-2-yl)amino]-3-methoxy-phenyl]-N-pyrrolidin-3-yl-acetamide;
2-[4-[(5-chloro-4-imidazo[1,2-a]pyridin-3-yl-pyrimidin-2-yl)amino]-3-methoxy-phenyl]-1-morpholino-ethanone;
2-[4-[(5-chloro-4-imidazo[1,2-a]pyridin-3-yl-pyrimidin-2-yl)amino]-3-methoxy-phenyl]-1-[4-(2-hydroxyethyl)piperazin-1-yl]ethanone;
2-[4-[(5-chloro-4-imidazo[1,2-a]pyridin-3-yl-pyrimidin-2-yl)amino]-3-methoxy-phenyl]-1-[(3R)-3-hydroxypyrrolidin-1-yl]ethanone;
1-[4-[4-[(5-chloro-4-imidazo[1,2-a]pyridin-3-yl-pyrimidin-2-yl)amino]-3-methoxy-phenyl]piperazin-1-yl]-2-methoxy-ethanone;
1-[4-[4-[(5-chloro-4-imidazo[1,2-a]pyridin-3-yl-pyrimidin-2-yl)amino]-3-methoxy-phenyl]piperazin-1-yl]-2-hydroxy-ethanone;
(2S)-1-[4-[4-[(5-chloro-4-imidazo[1,2-a]pyridin-3-yl-pyrimidin-2-yl)amino]-3-methoxy-phenyl]piperazin-1-yl]-2-hydroxy-propan-1-one;
1-[4-[4-[(5-chloro-4-imidazo[1,2-a]pyridin-3-yl-pyrimidin-2-yl)amino]-3-methoxy-phenyl]piperazin-1-yl]propan-1-one;
methyl 4-[4-[(5-chloro-4-imidazo[1,2-a]pyridin-3-yl-pyrimidin-2-yl)amino]-3-methoxy-phenyl]piperazine-1-carboxylate;
2-methoxyethyl 4-[4-[(5-chloro-4-imidazo[1,2-a]pyridin-3-yl-pyrimidin-2-yl)amino]-3-methoxy-phenyl]piperazine-1-carboxylate;
[4-[4-[(5-chloro-4-imidazo[1,2-a]pyridin-3-yl-pyrimidin-2-yl)amino]-3-methoxy-phenyl]piperazin-1-yl]-morpholino-methanone;
4-[4-[(5-chloro-4-imidazo[1,2-a]pyridin-3-yl-pyrimidin-2-yl)amino]-3-methoxy-phenyl]-N-(2-hydroxyethyl)-N-methyl-piperazine-1-carboxamide;
4-[4-[(5-chloro-4-imidazo[1,2-a]pyridin-3-yl-pyrimidin-2-yl)amino]-3-methoxy-phenyl]-N,N-dimethyl-piperazine-1-carboxamide;
[4-[4-[(5-chloro-4-imidazo[1,2-a]pyridin-3-yl-pyrimidin-2-yl)amino]-3-methoxy-phenyl]piperazin-1-yl]-piperazin-1-yl-methanone;
5-chloro-4-imidazo[1,2-a]pyridin-3-yl-N-[2-methoxy-4-[4-(2-methylsulfonylethyl)piperazin-1-yl]phenyl]pyrimidin-2-amine;
3-[4-[4-[(5-chloro-4-imidazo[1,2-a]pyridin-3-yl-pyrimidin-2-yl)amino]-3-methoxy-phenyl]piperazin-1-yl]propanenitrile;
(2S)-1-[4-[4-[(5-chloro-4-imidazo[1,2-a]pyridin-3-yl-pyrimidin-2-yl)amino]-3-methoxy-phenyl]piperazin-1-yl]propan-2-ol;
(2R)-1-[4-[4-[(5-chloro-4-imidazo[1,2-a]pyridin-3-yl-pyrimidin-2-yl)amino]-3-methoxy-phenyl]piperazin-1-yl]propan-2-ol;
1-[4-[4-[(5-chloro-4-imidazo[1,2-a]pyridin-3-yl-pyrimidin-2-yl)amino]-3-methoxy-phenyl]piperazin-1-yl]-2-methyl-propan-2-ol;
(2R)-3-[4-[4-[(5-chloro-4-imidazo[1,2-a]pyridin-3-yl-pyrimidin-2-yl)amino]-3-methoxy-phenyl]piperazin-1-yl]propane-1,2-diol;
4-[4-[(5-chloro-4-imidazo[1,2-a]pyridin-3-yl-pyrimidin-2-yl)amino]-3-methoxy-phenyl]piperazine-1-carboxamide;
4-[3-[(5-chloro-4-imidazo[1,2-a]pyridin-3-yl-pyrimidin-2-yl)amino]-4-methoxy-phenyl]piperazine-1-carboxamide;
(2S)-1-[4-[4-[(5-chloro-4-imidazo[1,2-a]pyridin-3-yl-pyrimidin-2-yl)amino]-3-methoxy-phenyl]piperazin-1-yl]-2,3-dihydroxy-propan-1-one;
4-[4-[(5-chloro-4-imidazo[1,2-a]pyridin-3-yl-pyrimidin-2-yl)amino]-3-methoxy-phenyl]-N,N-dimethyl-piperidine-1-carboxamide;
4-[4-[(5-chloro-4-imidazo[1,2-a]pyridin-3-yl-pyrimidin-2-yl)amino]-3-methoxy-phenyl]-N-(2-hydroxyethyl)-N-methyl-piperidine-1-carboxamide;
1-[4-[4-[(5-chloro-4-imidazo[1,2-a]pyridin-3-yl-pyrimidin-2-yl)amino]-3-methoxy-phenyl]-2-methyl-1-piperidyl]ethanone;
5-chloro-4-imidazo[1,2-a]pyridin-3-yl-N-[2-methoxy-4-(2-methyl-4-piperidyl)phenyl]pyrimidin-2-amine;
1-[3-[4-[(5-chloro-4-imidazo[1,2-a]pyridin-3-yl-pyrimidin-2-yl)amino]-3-methoxy-phenoxy]azetidin-1-yl]ethanone;
(2S)-1-[3-[4-[(5-chloro-4-imidazo[1,2-a]pyridin-3-yl-pyrimidin-2-yl)amino]-3-methoxy-phenoxy]azetidin-1-yl]propan-2-ol;
(2R)-1-[3-[4-[(5-chloro-4-imidazo[1,2-a]pyridin-3-yl-pyrimidin-2-yl)amino]-3-methoxy-phenoxy]azetidin-1-yl]propan-2-ol;
1-[3-[4-[(5-chloro-4-imidazo[1,2-a]pyridin-3-yl-pyrimidin-2-yl)amino]-3-methoxy-phenoxy]azetidin-1-yl]-2-methyl-propan-2-ol;
2-[3-[4-[(5-chloro-4-imidazo[1,2-a]pyridin-3-yl-pyrimidin-2-yl)amino]-3-methoxy-phenoxy]azetidin-1-yl]ethanol;
2-[4-[4-[(5-chloro-4-imidazo[1,2-a]pyridin-3-yl-pyrimidin-2-yl)amino]-3-methoxy-phenyl]piperazin-1-yl]-N,N-dimethyl-acetamide;
2-[4-[4-[(5-chloro-4-imidazo[1,2-a]pyridin-3-yl-pyrimidin-2-yl)amino]-3-methoxy-phenyl]piperazin-1-yl]-N-methyl-acetamide;
2-[4-[4-[(5-chloro-4-imidazo[1,2-a]pyridin-3-yl-pyrimidin-2-yl)amino]-3-methoxy-phenyl]piperazin-1-yl]-N-(2-hydroxyethyl)-N-methyl-acetamide;
3-[4-[(5-chloro-4-imidazo[1,2-a]pyridin-3-yl-pyrimidin-2-yl)amino]-3-methoxy-phenoxy]-N-(2-hydroxyethyl)-N-methyl-azetidine-1-carboxamide;
3-[4-[(5-chloro-4-imidazo[1,2-a]pyridin-3-yl-pyrimidin-2-yl)amino]-3-methoxy-phenoxy]-N,N-dimethyl-azetidine-1-carboxamide;
2-[4-[(5-chloro-4-imidazo[1,2-a]pyridin-3-yl-pyrimidin-2-yl)amino]-3-methoxy-phenyl]propane-1,3-diol;
N-[4-[(3R)-3-aminopyrrolidin-1-yl]-2-methoxy-phenyl]-5-chloro-4-imidazo[1,2-a]pyridin-3-yl-pyrimidin-2-amine;
1-[4-[4-[(5-fluoro-4-imidazo[1,2-a]pyridin-3-yl-pyrimidin-2-yl)amino]-3-methoxy-phenyl]piperazin-1-yl]ethanone;
5-fluoro-4-imidazo[1,2-a]pyridin-3-yl-N-(2-methoxy-4-piperazin-1-yl-phenyl)pyrimidin-2-amine;
4-[4-[(5-fluoro-4-imidazo[1,2-a]pyridin-3-yl-pyrimidin-2-yl)amino]-3-methoxy-phenyl]-N,N-dimethyl-piperazine-1-sulfonamide;
5-fluoro-4-imidazo[1,2-a]pyridin-3-yl-N-(2-methoxy-5-piperazin-1-yl-phenyl)pyrimidin-2-amine;
N-[4-[(3R)-3-(dimethylamino)pyrrolidin-1-yl]-2-methoxy-phenyl]-5-fluoro-4-imidazo[1,2-a]pyridin-3-yl-pyrimidin-2-amine;
5-fluoro-4-imidazo[1,2-a]pyridin-3-yl-N-[2-methoxy-5-(4-methoxy-1-piperidyl)phenyl]pyrimidin-2-amine;
1-[4-[4-[(5-fluoro-4-imidazo[1,2-a]pyridin-3-yl-pyrimidin-2-yl)amino]-3-methoxy-phenyl]-1-piperidyl]ethanone;
5-fluoro-4-imidazo[1,2-a]pyridin-3-yl-N-(2-methoxy-4-morpholino-phenyl)pyrimidin-2-amine;
5-fluoro-4-imidazo[1,2-a]pyridin-3-yl-N-(2-methoxy-4-pyrrolidin-1-yl-phenyl)pyrimidin-2-amine;
2-[4-[4-[(5-fluoro-4-imidazo[1,2-a]pyridin-3-yl-pyrimidin-2-yl)amino]-3-methoxy-phenyl]piperazin-1-yl]ethanol;
5-fluoro-4-imidazo[1,2-a]pyridin-3-yl-N-[2-methoxy-4-[4-(2-methoxyethyl)piperazin-1-yl]phenyl]pyrimidin-2-amine;
(3S)-1-[4-[(5-fluoro-4-imidazo[1,2-a]pyridin-3-yl-pyrimidin-2-yl)amino]-3-methoxy-phenyl]pyrrolidin-3-ol;
5-fluoro-4-imidazo[1,2-a]pyridin-3-yl-N-[2-methoxy-4-(1,4-oxazepan-4-yl)phenyl]pyrimidin-2-amine;
(3R)-1-[4-[(5-fluoro-4-imidazo[1,2-a]pyridin-3-yl-pyrimidin-2-yl)amino]-3-methoxy-phenyl]pyrrolidin-3-ol;
5-fluoro-4-imidazo[1,2-a]pyridin-3-yl-N-[2-methoxy-4-(4-piperidyl)phenyl]pyrimidin-2-amine;
5-fluoro-4-imidazo[1,2-a]pyridin-3-yl-N-[2-methoxy-4-[[(2R)-pyrrolidin-2-yl]methoxy]phenyl]pyrimidin-2-amine;
4-[4-[(5-fluoro-4-imidazo[1,2-a]pyridin-3-yl-pyrimidin-2-yl)amino]-3-methoxy-phenyl]piperazin-2-one;
(2S)-1-[4-[4-[(5-fluoro-4-imidazo[1,2-a]pyridin-3-yl-pyrimidin-2-yl)amino]-3-methoxy-phenyl]piperazin-1-yl]propan-2-ol;
(2R)-1-[4-[4-[(5-fluoro-4-imidazo[1,2-a]pyridin-3-yl-pyrimidin-2-yl)amino]-3-methoxy-phenyl]piperazin-1-yl]propan-2-ol;
1-[4-[4-[(5-fluoro-4-imidazo[1,2-a]pyridin-3-yl-pyrimidin-2-yl)amino]-3-methoxy-phenyl]piperazin-1-yl]-2-methyl-propan-2-ol;
1-[4-[4-[(4-imidazo[1,2-a]pyridin-3-yl-5-methoxy-pyrimidin-2-yl)amino]-3-methoxy-phenyl]piperazin-1-yl]ethanone;
4-imidazo[1,2-a]pyridin-3-yl-5-methoxy-N-(2-methoxy-4-piperazin-1-yl-phenyl)pyrimidin-2-amine;
1-[4-[4-[(4-imidazo[1,2-a]pyridin-3-yl-5-methoxy-pyrimidin-2-yl)amino]-3-methoxy-phenyl]-1-piperidyl]ethanone;
2-[4-[4-[(4-imidazo[1,2-a]pyridin-3-yl-5-methoxy-pyrimidin-2-yl)amino]-3-methoxy-phenyl]piperazin-1-yl]ethanol;
4-imidazo[1,2-a]pyridin-3-yl-5-methoxy-N-[2-methoxy-4-[[(2R)-pyrrolidin-2-yl]methoxy]phenyl]pyrimidin-2-amine;
1-[4-[4-[(4-imidazo[1,2-a]pyridin-3-yl-5-methyl-pyrimidin-2-yl)amino]-3-methoxy-phenyl]piperazin-1-yl]ethanone;
4-imidazo[1,2-a]pyridin-3-yl-N-(2-methoxy-4-piperazin-1-yl-phenyl)-5-methyl-pyrimidin-2-amine;
1-[4-[4-[(4-imidazo[1,2-a]pyridin-3-yl-5-methyl-pyrimidin-2-yl)amino]-3-methoxy-phenyl]-1-piperidyl]ethanone;
4-imidazo[1,2-a]pyridin-3-yl-N-(2-methoxy-4-morpholino-phenyl)-5-methyl-pyrimidin-2-amine;
2-[4-[4-[(4-imidazo[1,2-a]pyridin-3-yl-5-methyl-pyrimidin-2-yl)amino]-3-methoxy-phenyl]piperazin-1-yl]ethanol;
4-imidazo[1,2-a]pyridin-3-yl-N-[2-methoxy-4-[[(2R)-pyrrolidin-2-yl]methoxy]phenyl]-5-methyl-pyrimidin-2-amine;
1-[4-[4-[(4-imidazo[1,2-a]pyridin-3-yl-5-methyl-pyrimidin-2-yl)amino]-3-methoxy-phenoxy]-1-piperidyl]ethanone;
(2S)-1-[4-[4-[(4-imidazo[1,2-a]pyridin-3-yl-5-methyl-pyrimidin-2-yl)amino]-3-methoxy-phenyl]piperazin-1-yl]propan-2-ol;
(2R)-1-[4-[4-[(4-imidazo[1,2-a]pyridin-3-yl-5-methyl-pyrimidin-2-yl)amino]-3-methoxy-phenyl]piperazin-1-yl]propan-2-ol;
1-[4-[4-[(4-imidazo[1,2-a]pyridin-3-yl-5-methyl-pyrimidin-2-yl)amino]-3-methoxy-phenyl]piperazin-1-yl]-2-methyl-propan-2-ol;
1-[4-[4-[[4-imidazo[1,2-a]pyridin-3-yl-5-(trifluoromethyl)pyrimidin-2-yl]amino]-3-methoxy-phenyl]piperazin-1-yl]ethanone;
1-[4-[4-[[4-imidazo[1,2-a]pyridin-3-yl-5-(trifluoromethyl)pyrimidin-2-yl]amino]-3-methoxy-phenyl]-1-piperidyl]ethanone;
2-[4-[4-[[4-imidazo[1,2-a]pyridin-3-yl-5-(trifluoromethyl)pyrimidin-2-yl]amino]-3-methoxy-phenyl]piperazin-1-yl]ethanol;
1-[4-[4-[[4-imidazo[1,2-a]pyridin-3-yl-5-(trifluoromethyl)pyrimidin-2-yl]amino]-3-methoxy-phenoxy]-1-piperidyl]ethanone;
1-[4-[(5-fluoro-4-imidazo[1,2-a]pyridin-3-yl-pyrimidin-2-yl)amino]-3-methoxy-phenyl]piperidin-4-ol;
5-fluoro-4-imidazo[1,2-a]pyridin-3-yl-N-[2-methoxy-5-(4-piperidyl)phenyl]pyrimidin-2-amine;
5-fluoro-4-imidazo[1,2-a]pyridin-3-yl-N-(2-methoxy-4-tetrahydropyran-4-yl-phenyl)pyrimidin-2-amine;
1-[(2R)-2-[[4-[(4-imidazo[1,2-a]pyridin-3-yl-5-methoxy-pyrimidin-2-yl)amino]-3-methoxy-phenoxy]methyl]pyrrolidin-1-yl]ethanone;
2-[4-[4-[(5-bromo-4-imidazo[1,2-a]pyridin-3-yl-pyrimidin-2-yl)amino]-3-methoxy-phenyl]piperazin-1-yl]ethanol;
2-[4-[4-(2-hydroxyethyl)piperazin-1-yl]-2-methoxy-anilino]-4-imidazo[1,2-a]pyridin-3-yl-pyrimidine-5-carbonitrile;
1-[4-[4-[(5-bromo-4-imidazo[1,2-a]pyridin-3-yl-pyrimidin-2-yl)amino]-3-methoxy-phenyl]piperazin-1-yl]ethanone;
2-[4-(4-acetylpiperazin-1-yl)-2-methoxy-anilino]-4-imidazo[1,2-a]pyridin-3-yl-pyrimidine-5-carbonitrile;
5-fluoro-4-imidazo[1,2-a]pyridin-3-yl-N-[2-methoxy-4-(4-methylpiperazin-1-yl)phenyl]pyrimidin-2-amine;
N-[4-(4-ethylpiperazin-1-yl)-2-methoxy-phenyl]-5-fluoro-4-imidazo[1,2-a]pyridin-3-yl-pyrimidin-2-amine;
5-bromo-4-imidazo[1,2-a]pyridin-3-yl-N-[2-methoxy-4-(4-methylpiperazin-1-yl)phenyl]pyrimidin-2-amine;
5-bromo-N-[2-ethoxy-4-(4-methylpiperazin-1-yl)phenyl]-4-imidazo[1,2-a]pyridin-3-yl-pyrimidin-2-amine;
5-bromo-4-imidazo[1,2-a]pyridin-3-yl-N-[2-methoxy-4-(1-methylpyrrolidin-3-yl)oxy-phenyl]pyrimidin-2-amine;
5-bromo-N-(4,5-dimethoxy-2-methyl-phenyl)-4-imidazo[1,2-a]pyridin-3-yl-pyrimidin-2-amine;
1-[4-[4-[[5-chloro-4-[7-(hydroxymethyl)imidazo[1,2-a]pyridin-3-yl]pyrimidin-2-yl]amino]-3-methoxy-phenyl]piperazin-1-yl]ethanone;
1-[4-[4-[[4-(6-bromoimidazo[1,2-a]pyridin-3-yl)-5-chloro-pyrimidin-2-yl]amino]-3-methoxy-phenyl]piperazin-1-yl]ethanone;
3-[2-[4-(4-acetylpiperazin-1-yl)-2-methoxy-anilino]-5-chloro-pyrimidin-4-yl]imidazo[1,2-a]pyridine-6-carbonitrile;
1-[4-[4-[[5-chloro-4-(6,8-difluoroimidazo[1,2-a]pyridin-3-yl)pyrimidin-2-yl]amino]-3-methoxy-phenyl]piperazin-1-yl]ethanone;
1-[4-[4-[[5-chloro-4-(6-methylimidazo[1,2-a]pyridin-3-yl)pyrimidin-2-yl]amino]-3-methoxy-phenyl]piperazin-1-yl]ethanone;
1-[4-[4-[[5-chloro-4-[6-(dimethylamino)imidazo[1,2-a]pyridin-3-yl]pyrimidin-2-yl]amino]-3-methoxy-phenyl]piperazin-1-yl]ethanone;
1-[4-[4-[[5-chloro-4-(6-methoxyimidazo[1,2-a]pyridin-3-yl)pyrimidin-2-yl]amino]-3-methoxy-phenyl]piperazin-1-yl]ethanone;
3-[2-[4-(4-acetylpiperazin-1-yl)-2-methoxy-anilino]-5-chloro-pyrimidin-4-yl]imidazo[1,2-a]pyridine-7-carbonitrile;
1-[4-[4-[[5-chloro-4-(7-ethylimidazo[1,2-a]pyridin-3-yl)pyrimidin-2-yl]amino]-3-methoxy-phenyl]piperazin-1-yl]ethanone;
1-[4-[4-[[5-chloro-4-(7-fluoroimidazo[1,2-a]pyridin-3-yl)pyrimidin-2-yl]amino]-3-methoxy-phenyl]piperazin-1-yl]ethanone;
1-[4-[4-[[5-chloro-4-(7-methylimidazo[1,2-a]pyridin-3-yl)pyrimidin-2-yl]amino]-3-methoxy-phenyl]piperazin-1-yl]ethanone;
1-[4-[4-[[5-chloro-4-(7-methoxyimidazo[1,2-a]pyridin-3-yl)pyrimidin-2-yl]amino]-3-methoxy-phenyl]piperazin-1-yl]ethanone;
1-[4-[4-[[5-chloro-4-[8-(hydroxymethyl)imidazo[1,2-a]pyridin-3-yl]pyrimidin-2-yl]amino]-3-methoxy-phenyl]piperazin-1-yl]ethanone;
3-[2-[4-(4-acetylpiperazin-1-yl)-2-methoxy-anilino]-5-chloro-pyrimidin-4-yl]imidazo[1,2-a]pyridine-8-carbonitrile;
1-[4-[4-[[5-chloro-4-(8-fluoroimidazo[1,2-a]pyridin-3-yl)pyrimidin-2-yl]amino]-3-methoxy-phenyl]piperazin-1-yl]ethanone;
1-[4-[4-[[5-chloro-4-(8-methylimidazo[1,2-a]pyridin-3-yl)pyrimidin-2-yl]amino]-3-methoxy-phenyl]piperazin-1-yl]ethanone;
1-[4-[4-[[5-chloro-4-(8-methoxyimidazo[1,2-a]pyridin-3-yl)pyrimidin-2-yl]amino]-3-methoxy-phenyl]piperazin-1-yl]ethanone;
1-[4-[4-[[4-(8-aminoimidazo[1,2-a]pyridin-3-yl)-5-chloro-pyrimidin-2-yl]amino]-3-methoxy-phenyl]piperazin-1-yl]ethanone;
1-[4-[4-[[5-chloro-4-(7-chloroimidazo[1,2-a]pyridin-3-yl)pyrimidin-2-yl]amino]-3-methoxy-phenyl]piperazin-1-yl]ethanone;
1-[4-[4-[[4-(7-bromoimidazo[1,2-a]pyridin-3-yl)-5-chloro-pyrimidin-2-yl]amino]-3-methoxy-phenyl]piperazin-1-yl]ethanone;
1-[4-[4-[[5-chloro-4-(6-fluoroimidazo[1,2-a]pyridin-3-yl)pyrimidin-2-yl]amino]-3-methoxy-phenyl]piperazin-1-yl]ethanone;
1-[4-[4-[(5-chloro-4-pyrazolo[1,5-a]pyridin-3-yl-pyrimidin-2-yl)amino]-3-methoxy-phenyl]piperazin-1-yl]ethanone;
5-chloro-N-(2-methoxy-4-piperazin-1-yl-phenyl)-4-pyrazolo[1,5-a]pyridin-3-yl-pyrimidin-2-amine;
1-[4-[4-[(5-chloro-4-pyrazolo[1,5-a]pyridin-3-yl-pyrimidin-2-yl)amino]-3-methoxy-phenyl]piperazin-1-yl]-2-hydroxy-ethanone;
(2R)-1-[4-[4-[(5-chloro-4-pyrazolo[1,5-a]pyridin-3-yl-pyrimidin-2-yl)amino]-3-methoxy-phenyl]piperazin-1-yl]-2-hydroxy-propan-1-one;
(2S)-2-amino-1-[4-[4-[(5-chloro-4-pyrazolo[1,5-a]pyridin-3-yl-pyrimidin-2-yl)amino]-3-methoxy-phenyl]piperazin-1-yl]propan-1-one;
1-[4-[4-[(5-chloro-4-pyrazolo[1,5-a]pyridin-3-yl-pyrimidin-2-yl)amino]-3-methoxy-phenyl]piperazin-1-yl]-2-(methylamino)ethanone;
2-[4-[4-[(5-chloro-4-pyrazolo[1,5-a]pyridin-3-yl-pyrimidin-2-yl)amino]-3-methoxy-phenyl]piperazin-1-yl]ethanol;
(2S)-1-[3-[4-[(5-chloro-4-pyrazolo[1,5-a]pyridin-3-yl-pyrimidin-2-yl)amino]-3-methoxy-phenoxy]azetidin-1-yl]-2-hydroxy-propan-1-one;
(2R)-1-[3-[4-[(5-chloro-4-pyrazolo[1,5-a]pyridin-3-yl-pyrimidin-2-yl)amino]-3-methoxy-phenoxy]azetidin-1-yl]-2-hydroxy-propan-1-one;
5-chloro-N-[2-methoxy-4-(4-piperidyl)phenyl]-4-pyrazolo[1,5-a]pyridin-3-yl-pyrimidin-2-amine;
1-[4-[4-[(5-chloro-4-pyrazolo[1,5-a]pyridin-3-yl-pyrimidin-2-yl)amino]-3-methoxy-phenyl]-1-piperidyl]-2-hydroxy-ethanone;
(2R)-1-[4-[4-[(5-chloro-4-pyrazolo[1,5-a]pyridin-3-yl-pyrimidin-2-yl)amino]-3-methoxy-phenyl]-1-piperidyl]-2-hydroxy-propan-1-one;
(2S)-1-[4-[4-[(5-chloro-4-pyrazolo[1,5-a]pyridin-3-yl-pyrimidin-2-yl)amino]-3-methoxy-phenyl]-1-piperidyl]-2-hydroxy-propan-1-one;
1-[4-[4-[(5-chloro-4-imidazo[1,2-a]pyridin-3-yl-pyrimidin-2-yl)amino]-3-methoxy-phenyl]-1-piperidyl]-2-(methylamino)ethanone;
(2R)-2-amino-1-[4-[4-[(5-chloro-4-imidazo[1,2-a]pyridin-3-yl-pyrimidin-2-yl)amino]-3-methoxy-phenyl]-1-piperidyl]propan-1-one;
(2S)-2-amino-1-[4-[4-[(5-chloro-4-imidazo[1,2-a]pyridin-3-yl-pyrimidin-2-yl)amino]-3-methoxy-phenyl]-1-piperidyl]propan-1-one;
5-chloro-4-imidazo[1,2-a]pyridin-3-yl-N-[2-methoxy-4-(9-oxa-3,7-diazabicyclo[3.3.1]nonan-7-yl)phenyl]pyrimidin-2-amine;
2-[7-[4-[(5-chloro-4-imidazo[1,2-a]pyridin-3-yl-pyrimidin-2-yl)amino]-3-methoxy-phenyl]-9-oxa-3,7-diazabicyclo[3.3.1]nonan-3-yl]ethanol;
1-[4-[3-[(5-chloro-4-imidazo[1,2-a]pyridin-3-yl-pyrimidin-2-yl)amino]-4-methoxy-phenyl]piperazin-1-yl]-2-(methylamino)ethanone;
1-[4-[3-[(5-chloro-4-imidazo[1,2-a]pyridin-3-yl-pyrimidin-2-yl)amino]-4-methoxy-phenyl]piperazin-1-yl]-2-(dimethylamino)ethanone;
5-chloro-N-(2-methoxy-5-piperazin-1-yl-phenyl)-4-pyrazolo[1,5-a]pyridin-3-yl-pyrimidin-2-amine;
2-[4-[(5-chloro-4-pyrazolo[1,5-a]pyridin-3-yl-pyrimidin-2-yl)amino]-3-methoxy-phenyl]propane-1,3-diol;
5-chloro-N-[2-methoxy-4-(9-oxa-3,7-diazabicyclo[3.3.1]nonan-7-yl)phenyl]-4-pyrazolo[1,5-a]pyridin-3-yl-pyrimidin-2-amine;
2-[7-[4-[(5-chloro-4-pyrazolo[1,5-a]pyridin-3-yl-pyrimidin-2-yl)amino]-3-methoxy-phenyl]-9-oxa-3,7-diazabicyclo[3.3.1]nonan-3-yl]ethanol;
1-[4-[3-[(5-chloro-4-pyrazolo[1,5-a]pyridin-3-yl-pyrimidin-2-yl)amino]-4-methoxy-phenyl]piperazin-1-yl]-2-(methylamino)ethanone;
1-[4-[3-[(5-chloro-4-pyrazolo[1,5-a]pyridin-3-yl-pyrimidin-2-yl)amino]-4-methoxy-phenyl]piperazin-1-yl]-2-(dimethylamino)ethanone;
N-[3-[(5-chloro-4-pyrazolo[1,5-a]pyridin-3-yl-pyrimidin-2-yl)amino]-4-methoxy-phenyl]-2-(dimethylamino)acetamide;
1-[6-[(5-chloro-4-pyrazolo[1,5-a]pyridin-3-yl-pyrimidin-2-yl)amino]-5-methoxy-indolin-1-yl]ethanone;
N-(5-chloro-4-pyrazolo[1,5-a]pyridin-3-yl-pyrimidin-2-yl)-5-methoxy-indolin-6-amine;
1-[6-[(5-chloro-4-pyrazolo[1,5-a]pyridin-3-yl-pyrimidin-2-yl)amino]-5-methoxy-indolin-1-yl]-2-(methylamino)ethanone;
1-[6-[(5-chloro-4-pyrazolo[1,5-a]pyridin-3-yl-pyrimidin-2-yl)amino]-5-methoxy-indolin-1-yl]-2-(dimethylamino)ethanone;
1-[4-[3-methoxy-4-[(5-methyl-4-pyrazolo[1,5-a]pyridin-3-yl-pyrimidin-2-yl)amino]phenyl]piperazin-1-yl]ethanone;
N-(2-methoxy-4-piperazin-1-yl-phenyl)-5-methyl-4-pyrazolo[1,5-a]pyridin-3-yl-pyrimidin-2-amine;
1-[4-[3-methoxy-4-[(5-methyl-4-pyrazolo[1,5-a]pyridin-3-yl-pyrimidin-2-yl)amino]phenyl]piperazin-1-yl]-2-(methylamino)ethanone;
2-[4-[3-methoxy-4-[(5-methyl-4-pyrazolo[1,5-a]pyridin-3-yl-pyrimidin-2-yl)amino]phenyl]piperazin-1-yl]ethanol;
N-[2-methoxy-4-(4-piperidyl)phenyl]-5-methyl-4-pyrazolo[1,5-a]pyridin-3-yl-pyrimidin-2-amine;
2-hydroxy-1-[4-[3-methoxy-4-[(5-methyl-4-pyrazolo[1,5-a]pyridin-3-yl-pyrimidin-2-yl)amino]phenyl]-1-piperidyl]ethanone;
(2R)-2-hydroxy-1-[4-[3-methoxy-4-[(5-methyl-4-pyrazolo[1,5-a]pyridin-3-yl-pyrimidin-2-yl)amino]phenyl]-1-piperidyl]propan-1-one;
N-(2-methoxy-5-piperazin-1-yl-phenyl)-5-methyl-4-pyrazolo[1,5-a]pyridin-3-yl-pyrimidin-2-amine;
2-[7-[3-methoxy-4-[(5-methyl-4-pyrazolo[1,5-a]pyridin-3-yl-pyrimidin-2-yl)amino]phenyl]-9-oxa-3,7-diazabicyclo[3.3.1]nonan-3-yl]ethanol;
1-[4-[4-methoxy-3-[(5-methyl-4-pyrazolo[1,5-a]pyridin-3-yl-pyrimidin-2-yl)amino]phenyl]piperazin-1-yl]-2-(methylamino)ethanone;
1-[5-methoxy-6-[(5-methyl-4-pyrazolo[1,5-a]pyridin-3-yl-pyrimidin-2-yl)amino]indolin-1-yl]ethanone;
2-(dimethylamino)-1-[5-methoxy-6-[(5-methyl-4-pyrazolo[1,5-a]pyridin-3-yl-pyrimidin-2-yl)amino]indolin-1-yl]ethanone;
1-[4-[4-[(5-fluoro-4-pyrazolo[1,5-a]pyridin-3-yl-pyrimidin-2-yl)amino]-3-methoxy-phenyl]piperazin-1-yl]ethanone;
5-fluoro-N-(2-methoxy-4-piperazin-1-yl-phenyl)-4-pyrazolo[1,5-a]pyridin-3-yl-pyrimidin-2-amine;
1-[4-[4-[(5-fluoro-4-pyrazolo[1,5-a]pyridin-3-yl-pyrimidin-2-yl)amino]-3-methoxy-phenyl]piperazin-1-yl]-2-(methylamino)ethanone;
5-fluoro-N-[2-methoxy-4-(4-piperidyl)phenyl]-4-pyrazolo[1,5-a]pyridin-3-yl-pyrimidin-2-amine;
5-fluoro-N-(2-methoxy-5-piperazin-1-yl-phenyl)-4-pyrazolo[1,5-a]pyridin-3-yl-pyrimidin-2-amine;
2-[7-[4-[(5-fluoro-4-pyrazolo[1,5-a]pyridin-3-yl-pyrimidin-2-yl)amino]-3-methoxy-phenyl]-9-oxa-3,7-diazabicyclo[3.3.1]nonan-3-yl]ethanol;
2-(dimethylamino)-1-[6-[(5-fluoro-4-pyrazolo[1,5-a]pyridin-3-yl-pyrimidin-2-yl)amino]-5-methoxy-indolin-1-yl]ethanone;
1-[4-[4-[[5-chloro-4-(8-fluoroimidazo[1,2-a]pyridin-3-yl)pyrimidin-2-yl]amino]-3-methoxy-phenyl]-1-piperidyl]ethanone;
(2R)-1-[4-[4-[[5-chloro-4-(8-fluoroimidazo[1,2-a]pyridin-3-yl)pyrimidin-2-yl]amino]-3-methoxy-phenyl]piperazin-1-yl]-2-hydroxy-propan-1-one;
1-[4-[4-[[5-chloro-4-(8-fluoroimidazo[1,2-a]pyridin-3-yl)pyrimidin-2-yl]amino]-3-methoxy-phenyl]-1-piperidyl]-2-hydroxy-ethanone;
1-[4-[4-[[5-chloro-4-(8-fluoroimidazo[1,2-a]pyridin-3-yl)pyrimidin-2-yl]amino]-3-methoxy-phenyl]piperazin-1-yl]-2-hydroxy-ethanone;
2-[4-[[5-chloro-4-(8-fluoroimidazo[1,2-a]pyridin-3-yl)pyrimidin-2-yl]amino]-3-methoxy-phenyl]propane-1,3-diol;
1-[4-[4-[[5-chloro-4-(8-fluoroimidazo[1,2-a]pyridin-3-yl)pyrimidin-2-yl]amino]-3-methoxy-phenyl]-1-piperidyl]-2-(methylamino)ethanone;
5-chloro-4-(8-fluoroimidazo[1,2-a]pyridin-3-yl)-N-[2-methoxy-4-(9-oxa-3,7-diazabicyclo[3.3.1]nonan-7-yl)phenyl]pyrimidin-2-amine;
2-[7-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-3-yl]ethanol;
1-[4-[4-[[5-chloro-4-[7-(dimethylaminomethyl)imidazo[1,2-a]pyridin-3-yl]pyrimidin-2-yl]amino]-3-methoxy-phenyl]piperazin-1-yl]ethanone;
1-[4-[4-[[5-chloro-4-[7-(methylaminomethyl)imidazo[1,2-a]pyridin-3-yl]pyrimidin-2-yl]amino]-3-methoxy-phenyl]piperazin-1-yl]ethanone;
1-[4-[4-[[5-chloro-4-[7-(morpholinomethyl)imidazo[1,2-a]pyridin-3-yl]pyrimidin-2-yl]amino]-3-methoxy-phenyl]piperazin-1-yl]ethanone;
N-[[3-[2-[4-(4-acetylpiperazin-1-yl)-2-methoxy-anilino]-5-chloro-pyrimidin-4-yl]imidazo[1,2-a]pyridin-7-yl]methyl]-N-methyl-acetamide;
5-chloro-N-[2-methoxy-5-(9-oxa-3,7-diazabicyclo[3.3.1]nonan-7-yl)phenyl]-4-pyrazolo[1,5-a]pyridin-3-yl-pyrimidin-2-amine;
2-[[4-[(5-chloro-4-pyrazolo[1,5-a]pyridin-3-yl-pyrimidin-2-yl)amino]-3-methoxy-phenyl]methyl]propane-1,3-diol;
N-[3-[[5-chloro-4-(8-fluoroimidazo[1,2-a]pyridin-3-yl)pyrimidin-2-yl]amino]-4-methoxy-phenyl]-2-(dimethylamino)acetamide;
5-chloro-4-(8-fluoroimidazo[1,2-a]pyridin-3-yl)-N-[2-methoxy-5-(9-oxa-3,7-diazabicyclo[3.3.1]nonan-7-yl)phenyl]pyrimidin-2-amine;
2-[[4-[[5-chloro-4-(8-fluoroimidazo[1,2-a]pyridin-3-yl)pyrimidin-2-yl]amino]-3-methoxy-phenyl]methyl]propane-1,3-diol;
2-[4-[4-[[5-chloro-4-(8-fluoroimidazo[1,2-a]pyridin-3-yl)pyrimidin-2-yl]amino]-3-methoxy-phenyl]piperazin-1-yl]ethanol;
2-[4-[4-[[5-fluoro-4-(8-fluoroimidazo[1,2-a]pyridin-3-yl)pyrimidin-2-yl]amino]-3-methoxy-phenyl]piperazin-1-yl]ethanol;
1-[4-[4-[[4-(8-fluoroimidazo[1,2-a]pyridin-3-yl)-5-methyl-pyrimidin-2-yl]amino]-3-methoxy-phenyl]-1-piperidyl]ethanone;
4-(8-fluoroimidazo[1,2-a]pyridin-3-yl)-N-[2-methoxy-4-(4-piperidyl)phenyl]-5-methyl-pyrimidin-2-amine;
1-[4-[4-[[4-(8-fluoroimidazo[1,2-a]pyridin-3-yl)-5-methyl-pyrimidin-2-yl]amino]-3-methoxy-phenyl]-1-piperidyl]-2-hydroxy-ethanone;
4-(8-fluoroimidazo[1,2-a]pyridin-3-yl)-N-(2-methoxy-5-piperazin-1-yl-phenyl)-5-methyl-pyrimidin-2-amine;
(2R)-1-[4-[4-[[4-(8-fluoroimidazo[1,2-a]pyridin-3-yl)-5-methyl-pyrimidin-2-yl]amino]-3-methoxy-phenyl]-1-piperidyl]-2-hydroxy-propan-1-one;
1-[4-[4-[[5-cyclopropyl-4-(8-fluoroimidazo[1,2-a]pyridin-3-yl)pyrimidin-2-yl]amino]-3-methoxy-phenyl]piperazin-1-yl]ethanone;
1-[4-[4-[[5-cyclopropyl-4-(8-fluoroimidazo[1,2-a]pyridin-3-yl)pyrimidin-2-yl]amino]-3-methoxy-phenyl]-1-piperidyl]ethanone;
5-chloro-4-(8-fluoroimidazo[1,2-a]pyridin-3-yl)-N-[2-methoxy-4-(7-methyl-9-oxa-3,7-diazabicyclo[3.3.1]nonan-3-yl)phenyl]pyrimidin-2-amine;
3-[7-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-3-yl]propan-1-ol;
2-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-N,N-dimethyl-acetamide;
3-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-N-methyl-propanamide;
5-chloro-4-(8-fluoroimidazo[1,2-a]pyridin-3-yl)-N-[2-methoxy-4-[3-(2-methylsulfonylethyl)-9-oxa-3,7-diazabicyclo[3.3.1]nonan-7-yl]phenyl]pyrimidin-2-amine;
1-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-2-(methylamino)ethanone;
2-amino-1-[7-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-3-yl]ethanone;
(2S)-2-amino-1-[7-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-3-yl]-3-hydroxy-propan-1-one;
1-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-2-(dimethylamino)ethanone;
1-[7-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-3-yl]-2-hydroxy-ethanone;
(2S)-1-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-2-hydroxy-propan-1-one;
(2R)-1-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]-2-hydroxy-propan-1-one;
1-[3-[4-[[5-chloro-4-(8-fluoroimidazo[1,2-a]pyridin-3-yl)pyrimidin-2-yl]amino]-3-methoxy-phenyl]-9-oxa-3,7-diazabicyclo[3.3.1]nonan-7-yl]ethanone;
2-[4-[4-[[5-chloro-4-(8-fluoroimidazo[1,2-a]pyridin-3-yl)pyrimidin-2-yl]amino]-3-methoxy-phenyl]-1-piperidyl]-N,N-dimethyl-acetamide;
2-[4-[[5-chloro-4-(8-fluoroimidazo[1,2-a]pyridin-3-yl)pyrimidin-2-yl]amino]-3-methoxy-phenoxy]-N,N-dimethyl-acetamide;
(2S)-2-[4-[[5-chloro-4-(8-fluoroimidazo[1,2-a]pyridin-3-yl)pyrimidin-2-yl]amino]-3-methoxy-phenoxy]-N-methyl-propanamide;
(2R)-2-[4-[[5-chloro-4-(8-fluoroimidazo[1,2-a]pyridin-3-yl)pyrimidin-2-yl]amino]-3-methoxy-phenoxy]-N-methyl-propanamide;
N-[2-[4-[[5-chloro-4-(8-fluoroimidazo[1,2-a]pyridin-3-yl)pyrimidin-2-yl]amino]-3-methoxy-N-methyl-anilino]ethyl]-2-hydroxy-N-methyl-acetamide;
2-[2-[4-[[5-chloro-4-(8-fluoroimidazo[1,2-a]pyridin-3-yl)pyrimidin-2-yl]amino]-3-methoxy-N-methyl-anilino]ethyl-methyl-amino]ethanol;
1-[4-[[5-chloro-4-(8-fluoroimidazo[1,2-a]pyridin-3-yl)pyrimidin-2-yl]amino]-3-methoxy-phenyl]-N-methyl-piperidine-4-carboxamide;
2-[4-[[5-chloro-4-(8-fluoroimidazo[1,2-a]pyridin-3-yl)pyrimidin-2-yl]amino]-N-(2-hydroxyethyl)-3-methoxy-anilino]ethanol;
1-[4-[[5-chloro-4-(8-fluoroimidazo[1,2-a]pyridin-3-yl)pyrimidin-2-yl]amino]-3-methoxy-phenyl]piperazin-2-one;
2-[4-[[5-chloro-4-(8-fluoroimidazo[1,2-a]pyridin-3-yl)pyrimidin-2-yl]amino]-3-methoxy-N-methyl-anilino]-N,N-dimethyl-acetamide;
2-[4-[[5-chloro-4-(8-fluoroimidazo[1,2-a]pyridin-3-yl)pyrimidin-2-yl]amino]-3-methoxy-N-methyl-anilino]-N-methyl-acetamide;
2-[4-[[5-chloro-4-(8-fluoroimidazo[1,2-a]pyridin-3-yl)pyrimidin-2-yl]amino]-3-methoxy-N-methyl-anilino]-1-piperazin-1-yl-ethanone;
2-[4-[4-[(5-fluoro-4-imidazo[1,2-a]pyridin-3-yl-pyrimidin-2-yl)amino]-3-methoxy-phenyl]piperazin-1-yl]-2-methyl-propan-1-ol;
2-[4-[4-[(4-imidazo[1,2-a]pyridin-3-yl-5-methyl-pyrimidin-2-yl)amino]-3-methoxy-phenyl]piperazin-1-yl]-2-methyl-propan-1-ol;
2-[4-[4-[(5-chloro-4-pyrazolo[1,5-a]pyridin-3-yl-pyrimidin-2-yl)amino]-3-methoxy-phenyl]piperazin-1-yl]-2-methyl-propan-1-ol;
2-[4-[3-methoxy-4-[(5-methyl-4-pyrazolo[1,5-a]pyridin-3-yl-pyrimidin-2-yl)amino]phenyl]piperazin-1-yl]-2-methyl-propan-1-ol;
2-[7-[4-[(4-imidazo[1,2-a]pyridin-3-yl-5-methyl-pyrimidin-2-yl)amino]-3-methoxy-phenyl]-9-oxa-3,7-diazabicyclo[3.3.1]nonan-3-yl]ethanol;
1-[4-[3-methoxy-4-[(5-methyl-4-pyrazolo[1,5-a]pyridin-3-yl-pyrimidin-2-yl)amino]phenyl]-1-piperidyl]ethanone;
1-[4-[4-[(5-cyclopropyl-4-pyrazolo[1,5-a]pyridin-3-yl-pyrimidin-2-yl)amino]-3-methoxy-phenyl]piperazin-1-yl]ethanone;
(2R)-2-amino-1-[4-[3-methoxy-4-[(5-methyl-4-pyrazolo[1,5-a]pyridin-3-yl-pyrimidin-2-yl)amino]phenyl]piperazin-1-yl]propan-1-one;
2-(dimethylamino)-1-[4-[4-methoxy-3-[(5-methyl-4-pyrazolo[1,5-a]pyridin-3-yl-pyrimidin-2-yl)amino]phenyl]piperazin-1-yl]ethanone;
1-[4-[3-methoxy-4-[(5-methyl-4-pyrazolo[1,5-a]pyridin-3-yl-pyrimidin-2-yl)amino]phenyl]-1-piperidyl]-2-(methylamino)ethanone;
1-[4-[4-[(5-fluoro-4-pyrazolo[1,5-a]pyridin-3-yl-pyrimidin-2-yl)amino]-3-methoxy-phenyl]-1-piperidyl]-2-(methylamino)ethanone;
2-[4-(1-acetyl-4-piperidyl)-2-methoxy-anilino]-4-pyrazolo[1,5-a]pyridin-3-yl-pyrimidine-5-carbonitrile;
2-[4-[1-(2-hydroxyacetyl)-4-piperidyl]-2-methoxy-anilino]-4-pyrazolo[1,5-a]pyridin-3-yl-pyrimidine-5-carbonitrile;
1-[(1S,4S)-3-[4-[(5-chloro-4-pyrazolo[1,5-a]pyridin-3-yl-pyrimidin-2-yl)amino]-3-methoxy-phenyl]-3,6-diazabicyclo[2.2.1]heptan-6-yl]ethanone;
2-[7-[4-[(5-chloro-4-pyrazolo[1,5-a]pyridin-3-yl-pyrimidin-2-yl)amino]-3-methoxy-phenyl]-9-oxa-3,7-diazabicyclo[3.3.1]nonan-3-yl]propane-1,3-diol;
1-[4-[4-[[5-chloro-4-(7-pyrrolidin-1-ylimidazo[1,2-a]pyridin-3-yl)pyrimidin-2-yl]amino]-3-methoxy-phenyl]piperazin-1-yl]ethanone;
1-[4-[4-[[5-chloro-4-(6-fluoroimidazo[1,2-a]pyridin-3-yl)pyrimidin-2-yl]amino]-3-methoxy-phenyl]-1-piperidyl]-2-hydroxy-ethanone;
1-[4-[4-[[5-chloro-4-(7-methylimidazo[1,2-a]pyridin-3-yl)pyrimidin-2-yl]amino]-3-methoxy-phenyl]-1-piperidyl]-2-hydroxy-ethanone;
(2R)-1-[4-[4-[[5-chloro-4-(6-fluoroimidazo[1,2-a]pyridin-3-yl)pyrimidin-2-yl]amino]-3-methoxy-phenyl]-1-piperidyl]-2-hydroxy-propan-1-one;
5-chloro-4-(6-fluoroimidazo[1,2-a]pyridin-3-yl)-N-[2-methoxy-4-(4-piperidyl)phenyl]pyrimidin-2-amine;
1-[4-[4-[[5-chloro-4-(6,8-difluoroimidazo[1,2-a]pyridin-3-yl)pyrimidin-2-yl]amino]-3-methoxy-phenyl]piperazin-1-yl]-2-(methylamino)ethanone;
1-[4-[4-[[5-chloro-4-(6-fluoroimidazo[1,2-a]pyridin-3-yl)pyrimidin-2-yl]amino]-3-methoxy-phenyl]piperazin-1-yl]-2-hydroxy-ethanone;
1-[4-[4-[[5-chloro-4-(6,8-difluoroimidazo[1,2-a]pyridin-3-yl)pyrimidin-2-yl]amino]-3-methoxy-phenyl]piperazin-1-yl]-2-hydroxy-ethanone;
1-[4-[4-[[5-chloro-4-(7-methylimidazo[1,2-a]pyridin-3-yl)pyrimidin-2-yl]amino]-3-methoxy-phenyl]piperazin-1-yl]-2-hydroxy-ethanone;
(2R)-2-amino-1-[4-[4-[[5-chloro-4-(6-fluoroimidazo[1,2-a]pyridin-3-yl)pyrimidin-2-yl]amino]-3-methoxy-phenyl]piperazin-1-yl]propan-1-one;
(2R)-2-amino-1-[4-[4-[[5-chloro-4-(6,8-difluoroimidazo[1,2-a]pyridin-3-yl)pyrimidin-2-yl]amino]-3-methoxy-phenyl]piperazin-1-yl]propan-1-one;
(2R)-2-amino-1-[4-[4-[[5-chloro-4-(7-methylimidazo[1,2-a]pyridin-3-yl)pyrimidin-2-yl]amino]-3-methoxy-phenyl]piperazin-1-yl]propan-1-one;
(2R)-1-[4-[4-[[5-chloro-4-(6-fluoroimidazo[1,2-a]pyridin-3-yl)pyrimidin-2-yl]amino]-3-methoxy-phenyl]piperazin-1-yl]-2-hydroxy-propan-1-one;
(2R)-1-[4-[4-[[5-chloro-4-(6,8-difluoroimidazo[1,2-a]pyridin-3-yl)pyrimidin-2-yl]amino]-3-methoxy-phenyl]piperazin-1-yl]-2-hydroxy-propan-1-one;
(2R)-1-[4-[4-[[5-chloro-4-(7-methylimidazo[1,2-a]pyridin-3-yl)pyrimidin-2-yl]amino]-3-methoxy-phenyl]piperazin-1-yl]-2-hydroxy-propan-1-one;
(2S)-2-amino-1-[4-[4-[[5-chloro-4-(7-methylimidazo[1,2-a]pyridin-3-yl)pyrimidin-2-yl]amino]-3-methoxy-phenyl]piperazin-1-yl]propan-1-one;
2-[4-[4-[[5-chloro-4-(6-fluoroimidazo[1,2-a]pyridin-3-yl)pyrimidin-2-yl]amino]-3-methoxy-phenyl]piperazin-1-yl]-2-methyl-propan-1-ol;
2-[4-[4-[[4-(6-fluoroimidazo[1,2-a]pyridin-3-yl)-5-methyl-pyrimidin-2-yl]amino]-3-methoxy-phenyl]piperazin-1-yl]-2-methyl-propan-1-ol;
2-[4-[4-[[5-fluoro-4-(6-fluoroimidazo[1,2-a]pyridin-3-yl)pyrimidin-2-yl]amino]-3-methoxy-phenyl]piperazin-1-yl]-2-methyl-propan-1-ol;
2-[4-[3-methoxy-4-[[5-methyl-4-(7-methylimidazo[1,2-a]pyridin-3-yl)pyrimidin-2-yl]amino]phenyl]piperazin-1-yl]-2-methyl-propan-1-ol;
2-[4-[4-[[5-chloro-4-(6-fluoroimidazo[1,2-a]pyridin-3-yl)pyrimidin-2-yl]amino]-3-methoxy-phenyl]piperazin-1-yl]ethanol;
2-[4-[4-[[4-(6-fluoroimidazo[1,2-a]pyridin-3-yl)-5-methyl-pyrimidin-2-yl]amino]-3-methoxy-phenyl]piperazin-1-yl]ethanol;
5-chloro-4-(6,8-difluoroimidazo[1,2-a]pyridin-3-yl)-N-(2-methoxy-4-piperazin-1-yl-phenyl)pyrimidin-2-amine;
3-[5-chloro-2-(2-methoxy-4-piperazin-1-yl-anilino)pyrimidin-4-yl]imidazo[1,2-a]pyridine-6-carbonitrile;
(2S)-3-[4-[4-[(4-imidazo[1,2-a]pyridin-3-yl-5-methyl-pyrimidin-2-yl)amino]-3-methoxy-phenyl]piperazin-1-yl]propane-1,2-diol;
(2R)-3-[4-[4-[(4-imidazo[1,2-a]pyridin-3-yl-5-methyl-pyrimidin-2-yl)amino]-3-methoxy-phenyl]piperazin-1-yl]propane-1,2-diol;
2-[4-[4-[(4-imidazo[1,2-a]pyridin-3-yl-5-methyl-pyrimidin-2-yl)amino]-3-methoxy-phenyl]piperazin-1-yl]propane-1,3-diol;
2-[(2R)-4-[4-[(4-imidazo[1,2-a]pyridin-3-yl-5-methyl-pyrimidin-2-yl)amino]-3-methoxy-phenyl]-2-methyl-piperazin-1-yl]ethanol;
2-[(2S)-4-[4-[(4-imidazo[1,2-a]pyridin-3-yl-5-methyl-pyrimidin-2-yl)amino]-3-methoxy-phenyl]-2-methyl-piperazin-1-yl]ethanol;
2-[(2R)-4-[4-[(5-fluoro-4-imidazo[1,2-a]pyridin-3-yl-pyrimidin-2-yl)amino]-3-methoxy-phenyl]-2-methyl-piperazin-1-yl]ethanol;
2-[(2S)-4-[4-[(5-fluoro-4-imidazo[1,2-a]pyridin-3-yl-pyrimidin-2-yl)amino]-3-methoxy-phenyl]-2-methyl-piperazin-1-yl]ethanol;
2-[4-[4-[(4-imidazo[1,2-a]pyridin-3-yl-5-methyl-pyrimidin-2-yl)amino]-3-methoxy-phenyl]piperazin-1-yl]propan-1-ol;
1-[4-[4-[(5-chloro-4-imidazo[1,2-a]pyridin-3-yl-pyrimidin-2-yl)amino]-3-methoxy-phenyl]piperazin-1-yl]-2-morpholino-ethanone;
N-[2-[4-[(5-chloro-4-imidazo[1,2-a]pyridin-3-yl-pyrimidin-2-yl)amino]-3-methoxy-phenyl]-3-hydroxy-propyl]acetamide;
1-[3-[4-[(5-chloro-4-imidazo[1,2-a]pyridin-3-yl-pyrimidin-2-yl)amino]-3-methoxy-phenyl]azetidin-1-yl]ethanone;
1-[4-[4-[(5-chloro-4-imidazo[1,2-a]pyridin-3-yl-pyrimidin-2-yl)amino]-5-methoxy-2-methyl-phenyl]piperazin-1-yl]ethanone;
2-[4-[4-[(5-chloro-4-imidazo[1,2-a]pyridin-3-yl-pyrimidin-2-yl)amino]-5-methoxy-2-methyl-phenyl]piperazin-1-yl]ethanol;
1-[4-[4-[(5-chloro-4-imidazo[1,2-a]pyridin-3-yl-pyrimidin-2-yl)amino]-3-methoxy-2-methyl-phenyl]piperazin-1-yl]ethanone;
1-[4-[4-[(5-chloro-4-imidazo[1,2-a]pyridin-3-yl-pyrimidin-2-yl)amino]-2,3-dimethoxy-phenyl]piperazin-1-yl]ethanone;
1-[4-[4-[(5-chloro-4-imidazo[1,2-a]pyridin-3-yl-pyrimidin-2-yl)amino]-2-fluoro-3-methoxy-phenyl]piperazin-1-yl]ethanone;
6-(4-acetylpiperazin-1-yl)-3-[(5-chloro-4-imidazo[1,2-a]pyridin-3-yl-pyrimidin-2-yl)amino]-2-methoxy-benzonitrile;
1-[4-[4-[(5-chloro-4-imidazo[1,2-a]pyridin-3-yl-pyrimidin-2-yl)amino]-2,5-dimethoxy-phenyl]piperazin-1-yl]ethanone;
2-(4-acetylpiperazin-1-yl)-5-[(5-chloro-4-imidazo[1,2-a]pyridin-3-yl-pyrimidin-2-yl)amino]-4-methoxy-benzonitrile;
1-[4-[4-[(5-chloro-4-imidazo[1,2-a]pyridin-3-yl-pyrimidin-2-yl)amino]-3-(2-methoxyethoxy)phenyl]piperazin-1-yl]ethanone;
5-(4-acetylpiperazin-1-yl)-2-[(5-chloro-4-imidazo[1,2-a]pyridin-3-yl-pyrimidin-2-yl)amino]benzonitrile;
1-[4-[4-[(5-chloro-4-imidazo[1,2-a]pyridin-3-yl-pyrimidin-2-yl)amino]-3-(hydroxymethyl)phenyl]piperazin-1-yl]ethanone;
1-[4-[4-[(5-chloro-4-imidazo[1,2-a]pyridin-3-yl-pyrimidin-2-yl)amino]-3-(methoxymethyl)phenyl]piperazin-1-yl]ethanone;
5-chloro-4-imidazo[1,2-a]pyridin-3-yl-N-[2-methoxy-4-(3-oxa-7,9-diazabicyclo[3.3.1]nonan-7-yl)phenyl]pyrimidin-2-amine;
1-[3-[4-[(5-chloro-4-imidazo[1,2-a]pyridin-3-yl-pyrimidin-2-yl)amino]-3-methoxy-phenyl]-7-oxa-3,9-diazabicyclo[3.3.1]nonan-9-yl]ethanone;
and pharmaceutically acceptable salts thereof.

19. A pharmaceutical composition comprising a compound of formula (I), or a pharmaceutically acceptable salt thereof, according to claim 1 in association with a pharmaceutically acceptable adjuvant, diluent or carrier.

20. A method for treating cancer which comprises administering to a patient in need thereof a therapeutically effective amount of a compound of formula (I), or a pharmaceutically acceptable salt thereof, according to claim 1.
